(19) United States
(12) Patent Application Publication
(10) Pub. No.: US 2023/0070861 A1
(43) Pub. Date: Mar. 9, 2023

(54) COMPOSITIONS AND METHODS FOR TREATING HEPATITIS B

(71) Applicant: Beam Therapeutics Inc., Cambridge, MA (US)

(72) Inventors: Elena SMEKALOVA, Cambridge, MA (US); Michael PACKER, Cambridge, MA (US); Francine GREGOIRE, Cambridge, MA (US); Luis BARRERA, Cambridge, MA (US); Giuseppe CIARAMELLA, Cambridge, MA (US)

(73) Assignee: Beam Therapeutics Inc., Cambridge, MA (US)

(21) Appl. No.: 17/610,119

(22) PCT Filed: May 8, 2020
(86) PCT No.: PCT/US2020/032226

§ 371 Date: Nov. 9, 2021

Related U.S. Application Data
(60) Provisional application No. 62/927,585, filed on Oct. 29, 2019.
(60) Provisional application No. 62/846,422, filed on May 10, 2019.

(51) Int. Cl. A61K 31/7088; C07K 14/005; C12N 15/10; C12N 15/113; C12N 15/90; C12N 9/22; C12N 9/78; A61P 31/20

(52) U.S. Cl. CPC A61K 31/7088; C07K 14/005; C12N 15/102; C12N 15/1131; C12Y 305/04005; C12N 9/22; C12N 9/78; A61P 31/20; C12Y 305/04004; C12N 15/90

(57) ABSTRACT

The invention features compositions and methods for introducing mutations into the hepatitis B virus (HBV) genome.

### CROSS REFERENCE TO RELATED APPLICATIONS

This International PCT Application claims priority to and benefit of U.S. Provisional Application No. 62/846,422, filed on May 10, 2019 and U.S. Provisional Application No. 62/927,585, filed on Oct. 29, 2019, the contents of each of which are incorporated by reference herein in their entireties.

### BACKGROUND OF THE INVENTION

Hepatitis B is a serious liver infection caused by the hepatitis B virus (HBV). HBV is a small DNA hepadnavirus that replicates through an RNA intermediate and can persist in infected cells by integrating into a host's genome. Approximately 257 million people worldwide, including between 850,000 and 2.2 million people in the United States, are chronically infected with HBV. Chronic HBV infection manifests as chronic hepatitis, cirrhosis, and/or hepatocellular carcinoma. Between 20% and 30% of adults who have chronic HBV infection develop hepatocellular carcinoma or cirrhosis. HBV infection is responsible for between 600,000 and 1,000,000 deaths per year.

Current therapeutic approaches to HBV infection have severe limitations. Antiviral medications, e.g., tenofovir, a nucleotide reverse transcriptase inhibitor, can decrease viral replication but do not cure HBV infected patients. These antiviral therapies can cost patients as much as $500 to $1500 monthly. Due to the extent of liver damage caused by HBV, a transplant becomes necessary in some cases. In addition to the risks inherent in organ transplants, the cost can be prohibitive. Therefore, improved methods for treating HBV infection are urgently required.

### SUMMARY OF THE INVENTION

As described below, the present invention features compositions and methods for treating hepatitis B virus (HBV) infection by introducing alterations into the HBV genome. In particular embodiments, the invention provides a base editor system (e.g., a fusion protein comprising a programmable DNA binding protein, a nucleobase editor and gRNA) for modifying the HBV genome to introduce changes, such as premature stop codons or in the coding sequence of HBV or deamination of nucleobases in HBV covalently closed circular DNA (cccDNA).

Provided herein are methods and compositions for editing hepatitis B (HBV) genome and related treatment and uses thereof. In one aspect, provided herein is a method of editing a nucleobase of a hepatitis B virus (HBV) genome, in which the method comprises contacting the HBV genome with one or more guide RNAs and a base editor comprising a polynucleotide programmable DNA binding domain and an adenosine deaminase or cytidine deaminase domain, wherein said guide RNA targets said base editor to effect an alteration of the nucleobase of the HBV genome. In some embodiments, the nucleobase of the HBV genome in a polynucleotide encoding an HBV protein. In some embodiments, the contacting is in a eukaryotic cell, a mammalian cell, or a human cell. In some embodiments, the contacting is in a cell in vivo or ex vivo. In some embodiments, the cytidine deaminase converts a target C to U in the HBV genome. In some embodiments, the cytidine deaminase converts a target C·G to T·A in the polynucleotide encoding the HBV protein. In some embodiments, the adenosine deaminase converts a target A·T to G·C in the polynucleotide encoding the HBV protein.

In some embodiments of the above-delineated method, the alteration of the nucleobase in the HBV genome in the polynucleotide encoding the HBV protein results in a premature termination codon. In some embodiments, the alteration of the nucleobase results in an R87* or W120* termination in an HBV X protein. In some embodiments, the alteration of the nucleobase results in an W35* or W36* in an HBV S protein. In some embodiments, the alteration of the HBV polynucleotide is a missense mutation. In some embodiments, the missense mutation is in an HBV pol gene. In some embodiments, the missense mutation results in a E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P in an HBV polymerase protein encoded by the HBV pol gene. In some embodiments, the missense mutation is in an HBV core gene. In some embodiments, the missense mutation results in a T160A, T160A, P161F, S162L, C183R, or *184Q in an HBV core protein encoded by the HBV core gene. In some embodiments, the missense mutation is in an HBV X gene. In some embodiments, the missense mutation results in a H86R, W120R, E122K, E121K, or L141P in an HBV X protein encoded by the HBV X gene. In certain embodiments, the missense mutation results in a S38F, L39F, W35R, W36R, T37I, T37A, R78Q, S34L, F80P, or D33G in an HBV S protein encoded by the HBV S gene.

In some embodiments of the above-delineated method, the polynucleotide programmable DNA binding domain provided herein is a Cas9 selected from Streptococcus pyogenes Cas9 (SpCas9), Staphylococcus aureus Cas9 (SaCas9), Streptococcus thermophilus 1 Cas9 (St1Cas9), Streptococcus canis Cas9 (ScCas9), or variant thereof. In some embodiments, the Cas9 has protospacer-adjacent motif (PAM) specificity for a nucleic acid sequence selected from 5′-NGG-3′, 5′-NAG-3′, 5′-NGA-3′, 5′-NAA-3′, 5′-NNAGGA-3′, or 5′-NNACCA-3′. In some embodiments, the polynucleotide programmable DNA binding domain comprises a modified Cas9 having an altered protospacer-adjacent motif (PAM) specificity. In some embodiments, the altered PAM is selected from 5′-NNNRRT-3′, NGA-3′, 5′-NGCG-3′, 5′-NGN-3′, NGCN-3′, 5′-NGTN-3′, or 5′-NAA-3′. In some embodiments, the polynucleotide programmable DNA binding domain is a nuclease inactive or nickase variant. In some embodiments, the nuclease inactive or nickase variant is a nuclease inactivated Cas9 (dCas9) which comprises an amino acid substitution D10A or a corresponding amino acid substitution thereof.

In some embodiments of the above-delineated method, the adenosine deaminase domain is capable of deaminating adenine in deoxyribonucleic acid (DNA). In some embodiments, the adenosine deaminase is a TadA deaminase. In some embodiments, the TadA deaminase is TadA*7.10, TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the cytidine deaminase domain is capable of deaminating cytidine in DNA. In some embodiments, the cytidine deaminase is APOBEC or a derivative thereof. In some embodiments, the base editor further comprises a uracil glycosylase inhibitor (UGI). In some embodiments, the base editor does not comprise a uracil glycosylase inhibitor (UGI).

In some embodiments of the above-delineated method, the one or more guide RNAs for editing a nucleobase in the HBV genome comprises a CRISPR RNA (crRNA) and a trans-encoded small RNA (tracrRNA), wherein the crRNA comprises a nucleic acid sequence complementary to an HBV nucleic acid sequence. In some embodiments, the base editor is in complex with a single guide RNA (sgRNA) comprising a nucleic acid sequence complementary to an HBV nucleic acid sequence. In some embodiments, the HBV protein is the HBV S, polymerase (pol), core, or X protein.

In some aspects, the above-delineated method for editing a nucleobase of a hepatitis B virus (HBV) genome comprises editing one or more nucleobases. In some embodiments, the method comprises two or more guide RNAs that target two or more HBV nucleic acid sequences. In some embodiments, the guide RNAs comprise a sequence, from 5′ to 3′, or a 1, 2, 3, 4, or 5 nucleotide 5′ truncation fragment thereof, selected from one or more of

UCAAUCCCAACAAGGACACC;
GGGAACAAGAUCUACAGCAU;
AAGCCCAGGAUGAUGGGAUG;
CUGCCAACUGGAUCCUGCGC;
GACACAUCCAGCGAUAACCA;
GCUGCCAACUGGAUCCUGCG;
UAUGGAUGAUGUGGUAUUGG;
CCAUGCCCCAAAGCCACCCA;
AAGCCACCCAAGGCACAGCU;
GAGAAGUCCACCACGAGUCU;
CUUCUCUCAAUUUUCUAGGG;
GACGACGAGGCAGGUCCCCU;
CCCAACAAGGACACCUGGCC;
UGCCAACUGGAUCCUGCGCG;
AGGAGUUCCGCAGUAUGGAU;
CCGCAGUAUGGAUCGGCAGA;
CCUCUGCCGAUCCAUACUGC;
CGCCCACCGAAUGUUGCCCA;
GACUUCUCUCAAUUUUCUAG;
GUUCCGCAGUAUGGAUCGGC;
UACUAACAUUGAGGUUCCCG;
UCCGCAGUAUGGAUCGGCAG;
UCCUCUGCCGAUCCAUACUG;
GUAGCUCCAAAUUCUUUAUA;
or
AAUCCACACUCCGAAAGACA.

In another aspect, a method of treating hepatitis B virus (HBV) infection in a subject is provided, in which the method comprises administering to a subject in need thereof a fusion protein or polynucleotide encoding said fusion protein, the fusion protein comprising a polynucleotide programmable DNA binding domain and a base editor domain that is an adenosine deaminase or a cytidine deaminase domain and one or more guide polynucleotides that target the base editor domain to effect an A·T to G·C, C·G to T·A, or C·G to U·A alteration of the nucleic acid sequence encoding an HBV polypeptide.

In another aspect, a method of treating hepatitis B virus (HBV) infection in a subject is provided, in which the method comprises administering to a subject in need thereof one or more polynucleotides encoding a polynucleotide programmable DNA binding domain and a base editor domain that is an adenosine deaminase or a cytidine deaminase domain, and one or more guide polynucleotides that target the base editor domain to effect an A·T to G·C, C·G to T·A, or C·G to U·A alteration of the nucleic acid sequence encoding an HBV polypeptide.

In some embodiments of the above-delineated treatment methods, the subject is a mammal or a human. In some embodiments, the methods comprise delivering the fusion protein, the polynucleotide encoding said fusion protein, or the one or more polynucleotides encoding a polynucleotide programmable DNA binding domain and the base editor domain, and said one or more guide polynucleotides to a cell of the subject. In some embodiments, the cell is a hepatocyte. In some embodiments, the polynucleotide programmable DNA binding domain is a Cas9 selected from Streptococcus pyogenes Cas9 (SpCas9), Staphylococcus aureus Cas9 (SaCas9), Streptococcus thermophilus I Cas9 (St1Cas9), Streptococcus canis Cas9 (ScCas9), or a variant thereof. In some embodiments, the Cas9 has protospacer-adjacent motif (PAM) specificity for a nucleic acid sequence selected from 5′-NGG-3′, 5′-NAG-3′, 5′-NGA-3′, 5′-NAA-3′, 5′-NNAGGA-3′, or 5′-NNACCA-3′. In some embodiments, the polynucleotide programmable DNA binding domain comprises a modified Cas9 having an altered protospacer-adjacent motif (PAM) specificity. In some embodiments, the nucleic acid sequence of the altered PAM is selected from 5′-NNNRRT-3′, NGA-3′, 5′-NGCG-3′, 5′-NGN-3′, NGCN-3′, 5′-NGTN-3′, or 5′-NAA-3′. In some embodiments, the polynucleotide programmable DNA binding domain is a nuclease inactive or nickase variant. In some embodiments, the nuclease inactive or nickase variant is a nuclease inactivated Cas9 (dCas9) which comprises an amino acid substitution D10A or a corresponding amino acid substitution thereof. In some embodiments, the adenosine deaminase domain is capable of deaminating adenine in deoxyribonucleic acid (DNA). In some embodiments, adenosine deaminase is a TadA deaminase. In some embodiments, the TadA deaminase is TadA*7.10, TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In some embodiments, the cytidine deaminase domain is capable of deaminating cytidine in DNA. In some embodiments, the cytidine deaminase is APOBEC or a derivative thereof. In some embodiments, the base editor further comprises one or more uracil glycosylase inhibitors (UGIs). In some embodiments, the base editor does not comprise a uracil glycosylase inhibitor (UGI). In some embodiments, the one or more guide RNAs comprises a CRISPR RNA (crRNA) and a trans-encoded small RNA (tracrRNA), wherein the crRNA comprises a nucleic acid sequence complementary to an HBV nucleic acid sequence. In some embodiments, the base editor is in complex with a single guide RNA (sgRNA) comprising a nucleic acid sequence complementary to an HBV nucleic acid sequence. In some embodiment, the sgRNA comprises a nucleic acid sequence comprising at least 10 contiguous nucleotides that are complementary to the HBV nucleic acid sequence. In some embodiments, the sgRNA comprises a nucleic acid sequence comprising 15, 16, 17, 18, 19, 20, 21, 22, 23, 24, 25, 26, 27, 28, 29, 30, 31, 32, 33, 34, 35, 36, 37, 38, 39, or 40 contiguous nucleotides that are complementary to the HBV nucleic acid sequence.

In some embodiments, the above-delineated methods comprise editing one or more nucleobases. In some embodiments, the above described methods comprise two or more guide RNAs that target two or more HBV nucleic acid sequences. In some embodiments, the above-delineated methods comprise two or more guide RNAs that target three, four, or five HBV nucleic acid sequences. In some embodiments of the above-delineated methods, the HBV nucleic acid sequences encode one or more HBV proteins selected from HBV polymerase, HBV core protein, HBV S protein, HBV X protein, or a combination thereof. In some embodiments of the above-delineated methods, the one or more guide RNAs comprise a sequence, from 5′ to 3′, or a 1, 2, 3, 4, or 5 nucleotide 5′ truncation fragment thereof, selected from one or more of UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; or AAUCCACACUCCGAAAGACA. In some embodiments, the alteration of the polynucleotide encoding the HBV protein is a premature termination codon. In some embodiments, the alteration of the nucleic acid sequence results in an R87* or W120* in an HBV X protein encoded by the nucleic acid. In some embodiments, the alteration of the nucleic acid sequence results in a W35* or W36* in an HBV S protein encoded by the nucleic acid. In some embodiments, the alteration of the polynucleotide encoding the HBV protein is a missense mutation. In some embodiments, the missense mutation is in an HBV pol gene. In some embodiments, the missense mutation in the HBV pol gene results in a E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P in an HBV polymerase encoded by the HBV pol gene. In some embodiments, the missense mutation is in an HBV core gene. In some embodiments, the missense mutation in the HBV core gene results in a T160A, T160A, P161F, S162L, C183R, or *184Q in an HBV core protein encoded by the HBV core gene. In some embodiments, the missense mutation is in an HBV X gene. In some embodiments, the missense mutation results in a H86R, W120R, E122K, E121K, or L141P in an HBV X protein encoded by the HBV X gene. In some embodiments, the missense mutation is in an HBV S gene. In some embodiments, the missense mutation results in a S38F, L39F, W35R, W36R, T37I, T37A, R78Q, S34L, F80P, or D33G in an HBV S protein encoded by the HBV S gene. In some embodiments, the base editor is a BE4 or a variant of BE4 where APOBEC-1 is replaced with the sequence of APOBEC-3A, and/or Cas9 is replaced with a Cas9 variant comprising V1134, R1217, Q1334, and R1336 (termed SpCas9-VRQR).

In an aspect, compositions are provided, e.g., for treatment of HBV infection. In one aspect, composition is provided, in which the composition comprises a base editor bound to a guide RNA, wherein the guide RNA comprises a nucleic acid sequence that is complementary to an HBV gene. In an embodiment, the base editor is an adenosine deaminase or a cytidine deaminase. In an embodiment, the adenosine deaminase is capable of deaminating adenine in deoxyribonucleic acid (DNA). In an embodiment, the adenosine deaminase is a TadA deaminase. In an embodiment, the TadA deaminase is TadA*7.10, TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24. In an embodiment, the cytidine deaminase domain is capable of deaminating cytidine in DNA. In an embodiment, the cytidine deaminase is APOBEC or a derivative thereof. In an embodiment, the base editor further comprises one or more uracil glycosylase inhibitors (UGIs). In an embodiment, the base editor does not comprise a uracil glycosylase inhibitor (UGI). In an embodiment, the base editor (i) comprises a Cas9 nickase;

(ii) comprises a nuclease inactive Cas9;

(iii) does not comprise a UGI domain;

(iv) comprises an APOBEC-1 or APOBEC-3A cytidine deaminase;

(v) comprises an amino acid sequence that is at least 80%, 85%, 90%, 95%, 96%, 97%, 98%, 99%, or 100% identical to MSSETGPVAVDPTLRRRIEPHEFEVFFDPRELRKETCLLYEINWGGRHSIWRHTSQN TNKHVEVNFIEKFTTERYFCPNTRCSITWFLSWSPCGECSRAITEFLSRYPHVTLFIYIARL YHHADPRNRQGLRDLISSGVTIQIMTEQESGYCWRNFVNYSPSNEAHWPRYPHLWVRLYVLE LYCIILGLPPCLNILRRKQPQLTFFTIALQSCHYQRLPPHILWATGLKSGGSSGGSSGSETP GTSESATPESSGGSSGGSDKKYSIGLAIGTNSVGWAVITDEYKVPSKKFKVLGNTDRHSIKK NLIGALLFDSGETAEATRLKRTARRRYTRRKNRICYLQEIFSNEMAKVDDSFFHRLEESFLV EEDKKHERHPIFGNIVDEVAYHEKYPTIYHLRKKLVDSTDKADLRLIYLALAHMIKFRGHFL IEGDLNPDNSDVDKLFIQLVQTYNQLFEENPINASGVDAKAILSARLSKSRRLENLIAQLPG EKKNGLFGNLIALSLGLTPNFKSNFDLAEDAKLQLSKDTYDDDLDNLLAQIGDQYADLFLAA KNLSDAILLSDILRVNTEITKAPLSASMIKRYDEHHQDLTLLKALVRQQLPEKYKEIFFDQS KNGYAGYIDGGASQEEFYKFIKPILEKMDGTEELLVKLNREDLLRKQRTFDNGSIPHQIHLG ELHAILRRQEDFYPFLKDNREKIEKILTFRIPYYVGPLARGNSRFAWMTRKSEETITPWNFE EVVDKGASAQSFIERMTNFDKNLPNEKVLPKHSLLYEYFTVYNELTKVKYVTEGMRKPAFLS GEQKKAIVDLLFKTNRKVTVKQLKEDYFKKIECFDSVEISGVEDRFNASLGTYHDLLKIIKD KDFLDNEENEDILEDIVLTLTLFEDREMIEERLKTYAHLFDDKVMKQLKRRRYTGWGRLSRK LINGIRDKQSGKTILDFLKSDGFANRNFMQLIHDDSLTFKEDIQKAQVSGQGDSLHEHIANL AGSPAIKKGILQTVKVVDELVKVMGRHKPENIVIEMARENQTTQKGQKNSRERMKRIEEGIK ELGSQILKEHPVENTQLQNEKLYLYYLQNGRDMYVDQELDINRLSDYDVDAIVPQSFLKDDS IDNKVLTRSDKNRGKSDNVPSEEVVKKMKNYWRQLLNAKLITQRKFDNLTKAERGGLSELDK AGFIKRQLVETRQITKHVAQILDSRMNTKYDENDKLIREVKVITLKSKLVSDFRKDFQFYKV REINNYHHAHDAYLNAVVGTALIKKYPKLESEFVYGDYKVYDVRKMIAKSEQEIGKATAKYF FYSNIMNFFKTEITLANGEIRKRPLIETNGETGEIVWDKGRDFATVRKVLSMPQVNIVKKTE VQTGGFSKESILPKRNSDKLIARKKDWDPKKYGGFDSPTVAYSVLVVAKVEKGKSKKLKSVK ELLGITIMERSSFEKNPIDFLEAKGYKEVKKDLIIKLPKYSLFELENGRKRMLASAGELQKG NELALPSKYVNFLYLASHYEKLKGSPEDNEQKQLFVEQHKHYLDEIIEQISEFSKRVILADA NLDKVLSAYNKHRDKPIREQAENIIHLFTLTNLGAPAAFKYFDTTIDRKRYTSTKEVLDATL IHQSITGLYETRIDLSQLGGDSGGSKRTADGSEFESPKKKRKVE; or

(vi) comprises an amino acid sequence that is at least 80%, 85%, 90%, 95%, 96%, 97%, 98%, 99%, or 100% identical to MSSETGPVAVDPTLRRRIEPHEFEVFFDPRELRKETCLLYEINWGGRHSIWRHTSQNTNKHV EVNFIEKFTTERYFCPNTRCSITWFLSWSPCGECSRAITEFLSRYPHVTLFIYIARLYHHAD PRNRQGLRDLISSGVTIQIMTEQESGYCWRNFVNYSPSNEAHWPRYPHLWVRLYVLELYCII LGLPPCLNILRRKQPQLTFFTIALQSCHYQRLPPHILWATGLKSGGSSGGSSGSETPGTSES ATPESSGGSSGGSDKKYSIGLAIGTNSVGWAVITDEYKVPSKKFKVLGNTDRHSIKKNLIGA LLFDSGETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHFLIEGDL NPDNSDVDKLFIQLVQTYNQLFEENPINASGVDAKAILSARLSKSRRLENLIAQLPGEKKNG LFGNLIALSLGLTPNFKSNFDLAEDAKLQLSKDTYDDDLDNLLAQIGDQYADLFLAAKNLSD AILLSDILRVNTEITKAPLSASMIKRYDEHHQDLTLLKALVRQQLPEKYKEIFFDQSKNGYA GYIDGGASQEEFYKFIKPILEKMDGTEELLVKLNREDLLRKQRTFDNGSIPHQIHLGELHAI LRRQEDFYPFLKDNREKIEKILTFRIPYYVGPLARGNSRFAWMTRKSEETITPWNFEEVVDK GASAQSFIERMTNFDKNLPNEKVLPKHSLLYEYFTVYNELTKVKYVTEGMRKPAFLSGEQKK AIVDLLFKTNRKVTVKQLKEDYFKKIECFDSVEISGVEDRFNASLGTYHDLLKIIKDKDFLD NEENEDILEDIVLTLTLFEDREMIEERLKTYAHLFDDKVMKQLKRRRYTGWGRLSRKLINGI RDKQSGKTILDFLKSDGFANRNFMQLIHDDSLTFKEDIQKAQVSGQGDSLHEHIANLAGSPA IKKGILQTVKVVDELVKVMGRHKPENIVIEMARENQTTQKGQKNSRERMKRIEEGIKELGSQ ILKEHPVENTQLQNEKLYLYYLQNGRDMYVDQELDINRLSDYDVDHIVPQSFLKDDSIDNKV LTRSDKNRGKSDNVPSEEVVKKMKNYWRQLLNAKLITQRKFDNLTKAERGGLSELDKAGFIK RQLVETRQITKHVAQILDSRMNTKYDENDKLIREVKVITLKSKLVSDFRKDFQFYKVREINN YHHAHDAYLNAVVGTALIKKYPKLESEFVYGDYKVYDVRKMIAKSEQEIGKATAKYFFYSNI MNFFKTEITLANGEIRKRPLIETNGETGEIVWDKGRDFATVRKVLSMPQVNIVKKTEVQTGG FSKESILPKRNSDKLIARKKDWDPKKYGGFDSPTVAYSVLVVAKVEKGKSKKLKSVKELLGI TIMERSSFEKNPIDFLEAKGYKEVKKDLIIKLPKYSLFELENGRKRMLASAGELQKGNELAL PSKYVNFLYLASHYEKLKGSPEDNEQKQLFVEQHKHYLDEIIEQISEFSKRVILADANLDKV LSAYNKHRDKPIREQAENIIHLFTLTNLGAPAAFKYFDTTIDRKRYTSTKEVLDATLIHQSI TGLYETRIDLSQLGGDSGGSKRTADGSEFESPKKKRKVE.

In an embodiment, the guide RNA of the composition comprises a nucleic acid sequence that is complementary to an HBV gene encoding an HBV polymerase, HBV core protein, HBV S protein, or HBV X protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV X protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV S protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV polymerase. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV core protein. In an embodiment, the guide RNA comprises a 1, 2, 3, 4, or 5 nucleic acid truncation from the 5′ end of a nucleic acid selected from the group consisting of, from 5′ to 3′, UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; and AAUCCACACUCCGAAAGACA. In an embodiment, the guide RNA comprises a nucleic acid selected from the group consisting of, from 5′ to 3′, UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; and AAUCCACACUCCGAAAGACA. In an embodiment, the above-delineated composition further comprises a lipid. In an embodiment, the lipid is a cationic lipid. In an embodiment, the composition further comprises a pharmaceutically acceptable excipient.

In another aspect, a pharmaceutical composition is provided, in which the pharmaceutical composition comprises a base editor, or a nucleic acid encoding the base editor, and one or more guide RNAs (gRNAs) comprising a nucleic acid sequence complementary to an HBV gene in a pharmaceutically acceptable excipient. In an embodiment, the base editor (i) comprises a Cas9 nickase;

(ii) comprises a nuclease inactive Cas9;

(iii) does not comprise a UGI domain;

(iv) comprises an APOBEC-1 or APOBEC-3A cytidine deaminase;

(v) comprises an amino acid sequence that is at least 80%, 85%, 90%, 95%, 96%, 97%, 98%, 99%, or 100% identical to MSSETGPVAVDPTLRRRIEPHEFEVFFDPRELRKETCLLYEINWGGRHSIWRHTSQNTNKHV EVNFIEKFTTERYFCPNTRCSITWFLSWSPCGECSRAITEFLSRYPHVTLFIYIARLYHHAD PRNRQGLRDLISSGVTIQIMTEQESGYCWRNFVNYSPSNEAHWPRYPHLWVRLYVLELYCII LGLPPCLNILRRKQPQLTFFTIALQSCHYQRLPPHILWATGLKSGGSSGGSSGSETPGTSES ATPESSGGSSGGSDKKYSIGLAIGTNSVGWAVITDEYKVPSKKFKVLGNTDRHSIKKNLIGA LLFDSGETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHFLIEGDL NPDNSDVDKLFIQLVQTYNQLFEENPINASGVDAKAILSARLSKSRRLENLIAQLPGEKKNG LFGNLIALSLGLTPNFKSNFDLAEDAKLQLSKDTYDDDLDNLLAQIGDQYADLFLAAKNLSD AILLSDILRVNTEITKAPLSASMIKRYDEHHQDLTLLKALVRQQLPEKYKEIFFDQSKNGYA GYIDGGASQEEFYKFIKPILEKMDGTEELLVKLNREDLLRKQRTFDNGSIPHQIHLGELHAI LRRQEDFYPFLKDNREKIEKILTFRIPYYVGPLARGNSRFAWMTRKSEETITPWNFEEVVDK GASAQSFIERMTNFDKNLPNEKVLPKHSLLYEYFTVYNELTKVKYVTEGMRKPAFLSGEQKK AIVDLLFKTNRKVTVKQLKEDYFKKIECFDSVEISGVEDRFNASLGTYHDLLKIIKDKDFLD NEENEDILEDIVLTLTLFEDREMIEERLKTYAHLFDDKVMKQLKRRRYTGWGRLSRKLINGI RDKQSGKTILDFLKSDGFANRNFMQLIHDDSLTFKEDIQKAQVSGQGDSLHEHIANLAGSPA IKKGILQTVKVVDELVKVMGRHKPENIVIEMARENQTTQKGQKNSRERMKRIEEGIKELGSQ ILKEHPVENTQLQNEKLYLYYLQNGRDMYVDQELDINRLSDYDVDAIVPQSFLKDDSIDNKV LTRSDKNRGKSDNVPSEEVVKKMKNYWRQLLNAKLITQRKFDNLTKAERGGLSELDKAGFIK RQLVETRQITKHVAQILDSRMNTKYDENDKLIREVKVITLKSKLVSDFRKDFQFYKVREINN YHHAHDAYLNAVVGTALIKKYPKLESEFVYGDYKVYDVRKMIAKSEQEIGKATAKYFFYSNI MNFFKTEITLANGEIRKRPLIETNGETGEIVWDKGRDFATVRKVLSMPQVNIVKKTEVQTGG FSKESILPKRNSDKLIARKKDWDPKKYGGFDSPTVAYSVLVVAKVEKGKSKKLKSVKELLGI TIMERSSFEKNPIDFLEAKGYKEVKKDLIIKLPKYSLFELENGRKRMLASAGELQKGNELAL PSKYVNFLYLASHYEKLKGSPEDNEQKQLFVEQHKHYLDEIIEQISEFSKRVILADANLDKV LSAYNKHRDKPIREQAENIIHLFTLTNLGAPAAFKYFDTTIDRKRYTSTKEVLDATLIHQSI TGLYETRIDLSQLGGDSGGSKRTADGSEFESPKKKRKVE; or

(vi) comprises an amino acid sequence that is at least 80%, 85%, 90%, 95%, 96%, 97%, 98%, 99%, or 100% identical to MSSETGPVAVDPTLRRRIEPHEFEVFFDPRELRKETCLLYEINWGGRHSIWRHTSQNTNKHV EVNFIEKFTTERYFCPNTRCSITWFLSWSPCGECSRAITEFLSRYPHVTLFIYIARLYHHAD PRNRQGLRDLISSGVTIQIMTEQESGYCWRNFVNYSPSNEAHWPRYPHLWVRLYVLELYCII LGLPPCLNILRRKQPQLTFFTIALQSCHYQRLPPHILWATGLKSGGSSGGSSGSETPGTSES ATPESSGGSSGGSDKKYSIGLAIGTNSVGWAVITDEYKVPSKKFKVLGNTDRHSIKKNLIGA LLFDSGETAEATRLKRTARRRYTRRKNRICYLQEIFSNEMAKVDDSFFHRLEESFLVEEDKK HERHPIFGNIVDEVAYHEKYPTIYHLRKKLVDSTDKADLRLIYLALAHMIKFRGHFLIEGDL NPDNSDVDKLFIQLVQTYNQLFEENPINASGVDAKAILSARLSKSRRLENLIAQLPGEKKNG LFGNLIALSLGLTPNFKSNFDLAEDAKLQLSKDTYDDDLDNLLAQIGDQYADLFLAAKNLSD AILLSDILRVNTEITKAPLSASMIKRYDEHHQDLTLLKALVRQQLPEKYKEIFFDQSKNGYA GYIDGGASQEEFYKFIKPILEKMDGTEELLVKLNREDLLRKQRTFDNGSIPHQIHLGELHAI LRRQEDFYPFLKDNREKIEKILTFRIPYYVGPLARGNSRFAWMTRKSEETITPWNFEEVVDK GASAQSFIERMTNFDKNLPNEKVLPKHSLLYEYFTVYNELTKVKYVTEGMRKPAFLSGEQKK AIVDLLFKTNRKVTVKQLKEDYFKKIECFDSVEISGVEDRFNASLGTYHDLLKIIKDKDFLD NEENEDILEDIVLTLTLFEDREMIEERLKTYAHLFDDKVMKQLKRRRYTGWGRLSRKLINGI RDKQSGKTILDFLKSDGFANRNFMQLIHDDSLTFKEDIQKAQVSGQGDSLHEHIANLAGSPA IKKGILQTVKVVDELVKVMGRHKPENIVIEMARENQTTQKGQKNSRERMKRIEEGIKELGSQ ILKEHPVENTQLQNEKLYLYYLQNGRDMYVDQELDINRLSDYDVDHIVPQSFLKDDSIDNKV LTRSDKNRGKSDNVPSEEVVKKMKNYWRQLLNAKLITQRKFDNLTKAERGGLSELDKAGFIK RQLVETRQITKHVAQILDSRMNTKYDENDKLIREVKVITLKSKLVSDFRKDFQFYKVREINN YHHAHDAYLNAVVGTALIKKYPKLESEFVYGDYKVYDVRKMIAKSEQEIGKATAKYFFYSNI MNFFKTEITLANGEIRKRPLIETNGETGEIVWDKGRDFATVRKVLSMPQVNIVKKTEVQTGG FSKESILPKRNSDKLIARKKDWDPKKYGGFDSPTVAYSVLVVAKVEKGKSKKLKSVKELLGI TIMERSSFEKNPIDFLEAKGYKEVKKDLIIKLPKYSLFELENGRKRMLASAGELQKGNELAL PSKYVNFLYLASHYEKLKGSPEDNEQKQLFVEQHKHYLDEIIEQISEFSKRVILADANLDKV LSAYNKHRDKPIREQAENIIHLFTLTNLGAPAAFKYFDTTIDRKRYTSTKEVLDATLIHQSI TGLYETRIDLSQLGGDSGGSKRTADGSEFESPKKKRKVE.

In an embodiment, the base editor comprises a Cas9, or a Cas9 variant comprising V1134, R1217, Q1334, and R1336 (SpCas9-VRQR). In an embodiment, the gRNA and the base editor are formulated together or separately. In an embodiment, the gRNA comprises a nucleic acid sequence, from 5′ to 3′, or a 1, 2, 3, 4, or 5 nucleotide 5′ truncation fragment thereof, selected from one or more of UCAAUCCCAACAAGGACACC; GGGAACAAGAUCUACAGCAU AAGCCCAGGAUGAUGGGAUG; CUGCCAACUGGAUCCUGCGC GACACAUCCAGCGAUAACCA; GCUGCCAACUGGAUCCUGCG UAUGGAUGAUGUGGUAUUGG; CCAUGCCCCAAAGCCACCCA AAGCCACCCAAGGCACAGCU; GAGAAGUCCACCACGAGUCU CUUCUCUCAAUUUUCUAGGG; GACGACGAGGCAGGUCCCCU CCCAACAAGGACACCUGGCC; UGCCAACUGGAUCCUGCGCG AGGAGUUCCGCAGUAUGGAU; CCGCAGUAUGGAUCGGCAGA CCUCUGCCGAUCCAUACUGC; CGCCCACCGAAUGUUGCCCA GACUUCUCUCAAUUUUCUAG; GUUCCGCAGUAUGGAUCGGC UACUAACAUUGAGGUUCCCG; UCCGCAGUAUGGAUCGGCAG UCCUCUGCCGAUCCAUACUG; GUAGCUCCAAAUUCUUUAUA; or AAUCCACACUCCGAAAGACA. In an embodiment, the pharmaceutical composition further comprises a vector suitable for expression in a mammalian cell, wherein the vector comprises a polynucleotide encoding the base editor. In an embodiment, the vector is a viral vector. In an embodiment, the viral vector is a retroviral vector, adenoviral vector, lentiviral vector, herpesvirus vector, or adeno-associated viral vector (AAV). In an embodiment, the pharmaceutical composition further comprises a ribonucleoparticle suitable for expression in a mammalian cell.

In another aspect, a method of treating HBV infection is provided, in which the method comprises administering to a subject in need thereof the above-delineated composition or pharmaceutical composition.

Another aspect provides an HBV genome comprising an alteration selected from the group consisting of:

a premature termination codon introducing a R87STOP or W120STOP in the X gene;

a premature termination codon introducing a W35STOP or W36STOP in the S gene;

a missense mutation in the HBV pol gene that introduces a E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P in HBV polymerase;

a missense mutation is in the HBV core gene that introduces a T160A, T160A, P161F, S162L, C183R, or STOP184Q in the HBV Core polypeptide;

a missense mutation is in the X gene that introduces a H86R, W120R, E122K, E121K, or L141P in the HBV X polypeptide; and

a missense mutation in the S gene that introduces a S38F, L39F, W35R, W36R, T37I, T37A, R78Q, S34L, F80P, or D33G in the HBV S polypeptide.

In an embodiment, the HBV genome comprises two or more of the above described alterations.

In embodiments of the above-delineated methods, or the above-delineated HBV genome, the HBV is of genotype C or genotype D.

Provided in another aspect is a use of the composition of any of the above-delineated aspects and embodiments in the treatment of HBV infection in a subject.

Provided in another aspect is a use of the pharmaceutical composition of any of the above-delineated aspects and embodiments in the treatment of HBV infection in a subject.

In an embodiment of the above-delineated uses, the subject is a mammal. In an embodiment of the above-delineated uses, the subject is a human.

In an embodiment of the above-delineated methods or pharmaceutical compositions, the one or more guide RNAs are as listed in Table 26.

In another aspect, a guide RNA (gRNA) is provided which comprises a nucleic acid sequence that is complementary to an HBV gene. In an embodiment, the guide RNA comprises a nucleic acid sequence that is complementary to an HBV gene encoding an HBV polymerase, HBV core protein, HBV S protein, or HBV X protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV X protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV S protein. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV polymerase. In an embodiment, the guide RNA comprises 15, 16, 17, 18, 19, 20, 21, 22, 23, 24, 25, 26, 27, 28, 29, or 30 contiguous nucleotides that are perfectly complementary to an HBV gene that encodes an HBV core protein. In an embodiment, the guide RNA comprises a 1, 2, 3, 4, or 5 nucleic acid truncation from the 5′ end of a nucleic acid selected from the group consisting of, from 5′ to 3′, UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; and AAUCCACACUCCGAAAGACA. In an embodiment, the guide RNA comprises a nucleic acid selected from the group consisting of, from 5′ to 3′, UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; and AAUCCACACUCCGAAAGACA.

In another aspect, a pharmaceutical composition is provided, in which the pharmaceutical composition comprises (i) a nucleic acid encoding a base editor; and (ii) the guide RNA of any of the above-delineated aspects and embodiments. In an embodiment, the pharmaceutical composition further comprises a lipid. In an embodiment of the pharmaceutical composition, the nucleic acid encoding the base editor is an mRNA.

Compositions and articles defined by the invention were isolated or otherwise manufactured in connection with the examples provided below. Other features and advantages of the invention will be apparent from the detailed description, and from the claims.

### Definitions

The following definitions supplement those in the art and are directed to the current application and are not to be imputed to any related or unrelated case, e.g., to any commonly owned patent or application. Although any methods and materials similar or equivalent to those described herein can be used in the practice for testing of the present disclosure, the preferred materials and methods are described herein. Accordingly, the terminology used herein is for the purpose of describing particular embodiments only, and is not intended to be limiting.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

In this application, the use of the singular includes the plural unless specifically stated otherwise. It must be noted that, as used in the specification, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. It is contemplated that any embodiment discussed in this specification can be implemented with respect to any method or composition of the present disclosure, and vice versa. Furthermore, compositions of the present disclosure can be used to achieve methods of the present disclosure.

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Alternatively, particularly with respect to biological systems or processes, the term can mean within an order of magnitude, e.g., within 5-fold, within 2-fold of a value. Where particular values are described in the application and claims, unless otherwise stated, the term “about” means within an acceptable error range for the particular value should be assumed.

Reference in the specification to “some embodiments,” “an embodiment,” “one embodiment” or “other embodiments” means that a particular feature, structure, or characteristic described in connection with the embodiments is included in at least some embodiments, but not necessarily all embodiments, of the present disclosures.

By “adenosine deaminase” is meant a polypeptide or fragment thereof capable of catalyzing the hydrolytic deamination of adenine or adenosine. In some embodiments, the deaminase or deaminase domain is an adenosine deaminase catalyzing the hydrolytic deamination of adenosine to inosine or deoxy adenosine to deoxyinosine. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium.

In some embodiments, the deaminase or deaminase domain is a variant of a naturally-occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase. In some embodiments, the adenosine deaminase is from a bacterium, such as, E. coli, S. aureus, S. typhi, S. putrefaciens, H. influenzae, or C. crescentus. In some embodiments, the adenosine deaminase is a TadA deaminase. In some embodiments, the TadA deaminase is an E. coli TadA (ecTadA) deaminase or a fragment thereof.

For example, deaminase domains are described in International PCT Application Nos. PCT/2017/045381 (WO 2018/027078) and PCT/US2016/058344 (WO 2017/070632), each of which is incorporated herein by reference for its entirety. Also, see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017)), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

A wild type TadA(wt) adenosine deaminase has the following sequence (also termed TadA reference sequence):

MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD.

In some embodiments, the adenosine deaminase comprises an alteration in the following sequence:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD (also termed TadA*7.10).

In some embodiments, TadA*7.10 comprises at least one alteration. In some embodiments, TadA*7.10 comprises an alteration at amino acid 82 and/or 166. In particular embodiments, a variant of the above-referenced sequence comprises one or more of the following alterations: Y147T, Y147R, Q154S, Y123H, V82S, T166R, and/or Q154R. The alteration Y123H refers to the alteration H123Y in TadA*7.10 reverted back to Y123H TadA(wt). In other embodiments, a variant of the TadA*7.10 sequence comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In other embodiments, the invention provides adenosine deaminase variants that include deletions, e.g., TadA*8, comprising a deletion of the C-terminus beginning at residue 149, 150, 151, 152, 153, 154, 155, 156, or 157, relative to TadA*7.10, the TadA reference sequence, or a corresponding mutation in another TadA. In other embodiments, the adenosine deaminase variant is a TadA (e.g., TadA*8) monomer comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a TadA (e.g., TadA*8) monomer comprising the following alterations: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In still other embodiments, the adenosine deaminase variant is a homodimer comprising two adenosine deaminase domains each having one or more of the following alterations Y147T, Y147R, Q154S, Y123H, V82S, T166R, and/or Q154R relative to TadA*7.10, the TadA reference sequence, or a corresponding mutation in another TadA. In other embodiments, the adenosine deaminase variant is a homodimer comprising two adenosine deaminase domains (e.g., TadA*8) each having a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA. In other embodiments, the adenosine deaminase variant is a heterodimer comprising a wild-type TadA adenosine deaminase domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer comprising a wild-type TadA adenosine deaminase domain and an adenosine deaminase variant domain (e.g. TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In other embodiments, the adenosine deaminase variant is a heterodimer comprising a TadA*7.10 domain and an adenosine deaminase variant domain (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant is a heterodimer comprising a TadA*7.10 domain and an adenosine deaminase variant domain (e.g. TadA*8) comprising a combination of the following alterations: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; or I76Y+V82S+Y123H+Y147R+Q154R, relative to TadA*7.10, the TadA reference sequence, or a corresponding mutation in another TadA. In one embodiment, the adenosine deaminase is a TadA*8 that comprises or consists essentially of the following sequence or a fragment thereof having adenosine deaminase activity:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSID.

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8. In particular embodiments, an adenosine deaminase heterodimer comprises a TadA*8 domain and an adenosine deaminase domain selected from one of the following:

Staphylococcus aureus (S. aureus) TadA:
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN
Bacillus subtilis (B. subtilis) TadA:
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE
Salmonella typhimurium (S. typhimurium) TadA:
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV
Shewanella putrefaciens (S. putrefaciens) TadA:
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE
Haemophilus influenzae F3031 (H. influenzae)
TadA:
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK
Caulobacter crescentus (C. crescentus) TadA:
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI
Geobacter sulfurreducens (G. sulfurreducens) TadA:
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP
TadA*7.10
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD.

By “Adenosine Deaminase Base Editor 8 (ABE8) polypeptide” or “ABE8” is meant a base editor as defined herein comprising an adenosine deaminase variant comprising an alteration at amino acid position 82 and/or 166 of the following reference sequence:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, ABE8 comprises further alterations, as described herein, relative to the reference sequence.

By “Adenosine Deaminase Base Editor 8 (ABE8) polynucleotide” is meant a polynucleotide encoding an ABE8.

“Administering” is referred to herein as providing one or more compositions described herein to a patient or a subject. By way of example and without limitation, composition administration, e.g., injection, can be performed by intravenous (i.v.) injection, sub-cutaneous (s.c.) injection, intradermal (i.d.) injection, intraperitoneal (i.p.) injection, or intramuscular (i.m.) injection. One or more such routes can be employed. Parenteral administration can be, for example, by bolus injection or by gradual perfusion over time. Alternatively, or concurrently, administration can be by an oral route.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, and a 50% or greater change in expression levels.

By “ameliorate” is meant decrease, suppress, attenuate, diminish, arrest, or stabilize the development or progression of a disease.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “base editor (BE),” or “nucleobase editor (NBE)” is meant an agent that binds a polynucleotide and has nucleobase modifying activity. In various embodiments, the base editor comprises a nucleobase modifying polypeptide (e.g., a deaminase) and a polynucleotide programmable nucleotide binding domain in conjunction with a guide polynucleotide (e.g., guide RNA). In various embodiments, the agent is a biomolecular complex comprising a protein domain having base editing activity, i.e., a domain capable of modifying a base (e.g., A, T, C, G, or U) within a nucleic acid molecule (e.g., DNA). In some embodiments, the polynucleotide programmable DNA binding domain is fused or linked to a deaminase domain. In one embodiment, the agent is a fusion protein comprising one or more domains having base editing activity. In another embodiment, the protein domains having base editing activity are linked to the guide RNA (e.g., via an RNA binding motif on the guide RNA and an RNA binding domain fused to the deaminase). In some embodiments, the domains having base editing activity are capable of deaminating a base within a nucleic acid molecule. In some embodiments, the base editor is capable of deaminating one or more bases within a DNA molecule. In some embodiments, the base editor is capable of deaminating a cytosine (C) or an adenosine (A) within DNA. In some embodiments, the base editor is capable of deaminating a cytosine (C) and an adenosine (A) within DNA. In some embodiments, the base editor is a cytidine base editor (CBE). In some embodiments, the base editor is an adenosine base editor (ABE). In some embodiments, the base editor is an adenosine base editor (ABE) and a cytidine base editor (CBE). In some embodiments, the base editor is a nuclease-inactive Cas9 (dCas9) fused to an adenosine deaminase. In some embodiments, the Cas9 is a circular permutant Cas9 (e.g., spCas9 or saCas9). Circular permutant Cas9s are known in the art and described, for example, in Oakes et al., Cell 176, 254-267, 2019. In some embodiments, the base editor is fused to an inhibitor of base excision repair, for example, a UGI domain, or a dISN domain. In some embodiments, the fusion protein comprises a Cas9 nickase fused to a deaminase and an inhibitor of base excision repair, such as a UGI or dISN domain. In other embodiments the base editor is an abasic base editor.

In some embodiments, an adenosine deaminase is evolved from TadA. In some embodiments, the polynucleotide programmable DNA binding domain is a CRISPR associated (e.g., Cas or Cpf1) enzyme. In some embodiments, the base editor is a catalytically dead Cas9 (dCas9) fused to a deaminase domain. In some embodiments, the base editor is a Cas9 nickase (nCas9) fused to a deaminase domain. In some embodiments, the base editor is fused to an inhibitor of base excision repair (BER). In some embodiments, the inhibitor of base excision repair is a uracil DNA glycosylase inhibitor (UGI). In some embodiments, the inhibitor of base excision repair is an inosine base excision repair inhibitor. Details of base editors are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); Komor, A. C., et al., “Improved base excision repair inhibition and bacteriophage Mu Gam protein yields C:G-to-T:A base editors with higher efficiency and product purity” Science Advances 3:eaao4774 (2017), and Rees, H. A., et al., “Base editing: precision chemistry on the genome and transcriptome of living cells.” Nat Rev Genet. 2018 December; 19(12):770-788. doi: 10.1038/s41576-018-0059-1, the entire contents of which are hereby incorporated by reference.

In some embodiments, base editors are generated (e.g., ABE8) by cloning an adenosine deaminase variant (e.g., TadA*8) into a scaffold that includes a circular permutant Cas9 (e.g., spCAS9) and a bipartite nuclear localization sequence. Circular permutant Cas9s are known in the art and described, for example, in Oakes et al., Cell 176, 254-267, 2019. Exemplary circular permutant sequences are set forth below, in which the bold sequence indicates sequence derived from Cas9, the italics sequence denotes a linker sequence, and the underlined sequence denotes a bipartite nuclear localization sequence.

CP5 (with MSP “NGC=Pam Variant with mutations Regular Cas9 likes NGG” PID=Protein Interacting Domain and “D10A” nickase):

EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFMQPTVAYSVLVVAKVEKGKSKKLKSVKELLGITIMERSSFEKN
PIDFLEAKGYKEVKKDLIIKLPKYSLFELENGRKRMLASAKFLQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPRAFKYF
DTTIARKEYRSTKEVLDATLIHQSITGLYETRIDLSQLGGDGGSGGSGGS
GGSGGSGGSGGMDKKYSIGLAIGTNSVGWAVITDEYKVPSKKFKVLGNTD
RHSIKKNLIGALLFDSGETAEATRLKRTARRRYTRRKNRICYLQEIFSNE
MAKVDDSFFHRLEESFLVEEDKKHERHPIFGNIVDEVAYHEKYPTIYHLR
KKLVDSTDKADLRLIYLALAHMIKFRGHFLIEGDLNPDNSDVDKLFIQLV
QTYNQLFEENPINASGVDAKAILSARLSKSRRLENLIAQLPGEKKNGLFG
NLIALSLGLTPNFKSNFDLAEDAKLQLSKDTYDDDLDNLLAQIGDQYADL
FLAAKNLSDAILLSDILRVNTEITKAPLSASMIKRYDEHHQDLTLLKALV
RQQLPEKYKEIFFDQSKNGYAGYIDGGASQEEFYKFIKPILEKMDGTEEL
LVKLNREDLLRKQRTFDNGSIPHQIHLGELHAILRRQEDFYPFLKDNREK
IEKILTFRIPYYVGPLARGNSRFAWMTRKSEETITPWNFEEVVDKGASAQ
SFIERMTNFDKNLPNEKVLPKHSLLYEYFTVYNELTKVKYVTEGMRKPAF
LSGEQKKAIVDLLFKTNRKVTVKQLKEDYFKKIECFDSVEISGVEDRFNA
SLGTYHDLLKIIKDKDFLDNEENEDILEDIVLTLTLFEDREMIEERLKTY
AHLFDDKVMKQLKRRRYTGWGRLSRKLINGIRDKQSGKTILDFLKSDGFA
NRNFMQLIHDDSLTFKEDIQKAQVSGQGDSLHEHIANLAGSPAIKKGILQ
TVKVVDELVKVMGRHKPENIVIEMARENQTTQKGQKNSRERMKRIEEGIK
ELGSQILKEHPVENTQLQNEKLYLYYLQNGRDMYVDQELDINRLSDYDVD
HIVPQSFLKDDSIDNKVLTRSDKNRGKSDNVPSEEVVKKMKNYWRQLLNA
KLITQRKFDNLTKAERGGLSELDKAGFIKRQLVETRQITKHVAQILDSRM
NTKYDENDKLIREVKVITLKSKLVSDFRKDFQFYKVREINNYHHAHDAYL
NAVVGTALIKKYPKLESEFVYGDYKVYDVRKMIAKSEQEGADKRTADGSE
FESPKKKRKV*

In some embodiments, the ABE8 is selected from a base editor from Table 8 infra. In some embodiments, ABE8 contains an adenosine deaminase variant evolved from TadA. In some embodiments, the adenosine deaminase variant of ABE8 is a TadA*8 variant as described in Table 8 infra. In some embodiments, the adenosine deaminase variant is the TadA*7.10 variant (e.g., TadA*8) comprising one or more of an alteration selected from the group consisting of Y147T, Y147R, Q154S, Y123H, V82S, T166R, and/or Q154R. In various embodiments, ABE8 comprises TadA*7.10 variant (e.g. TadA*8) with a combination of alterations selected from the group of Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R.

In some embodiments ABE8 is a monomeric construct. In some embodiments, ABE8 is a heterodimeric construct. In some embodiments the ABE8 base editor comprises the sequence:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTD

By way of example, the adenine base editor ABE to be used in the base editing compositions, systems and methods described herein has the nucleic acid sequence (8877 base pairs), (Addgene, Watertown, Mass.; Gaudelli N M, et al., Nature. 2017 November 23; 551(7681):464-471. doi: 10.1038/nature24644; Koblan L W, et al., Nat Biotechnol. 2018 October; 36(9):843-846. doi: 10.1038/nbt. 4172.) as provided below. Polynucleotide sequences having at least 95% or greater identity to the ABE nucleic acid sequence are also encompassed.

ATATGCCAAGTACGCCCCCTATTGACGTCAATGACGGTAAATGGCCCGCCTGGCATTATGCCCAGTACAT
GACCTTATGGGACTTTCCTACTTGGCAGTACATCTACGTATTAGTCATCGCTATTACCATGGTGATGCGG
TTTTGGCAGTACATCAATGGGCGTGGATAGCGGTTTGACTCACGGGGATTTCCAAGTCTCCACCCCATTG
ACGTCAATGGGAGTTTGTTTTGGCACCAAAATCAACGGGACTTTCCAAAATGTCGTAACAACTCCGCCCC
ATTGACGCAAATGGGCGGTAGGCGTGTACGGTGGGAGGTCTATATAAGCAGAGCTGGTTTAGTGAACCGT
CAGATCCGCTAGAGATCCGCGGCCGCTAATACGACTCACTATAGGGAGAGCCGCCACCATGAAACGGACA
GCCGACGGAAGCGAGTTCGAGTCACCAAAGAAGAAGCGGAAAGTCTCTGAAGTCGAGTTTAGCCACGAGT
ATTGGATGAGGCACGCACTGACCCTGGCAAAGCGAGCATGGGATGAAAGAGAAGTCCCCGTGGGCGCCGT
GCTGGTGCACAACAATAGAGTGATCGGAGAGGGATGGAACAGGCCAATCGGCCGCCACGACCCTACCGCA
CACGCAGAGATCATGGCACTGAGGCAGGGAGGCCTGGTCATGCAGAATTACCGCCTGATCGATGCCACCC
TGTATGTGACACTGGAGCCATGCGTGATGTGCGCAGGAGCAATGATCCACAGCAGGATCGGAAGAGTGGT
GTTCGGAGCACGGGACGCCAAGACCGGCGCAGCAGGCTCCCTGATGGATGTGCTGCACCACCCCGGCATG
AACCACCGGGTGGAGATCACAGAGGGAATCCTGGCAGACGAGTGCGCCGCCCTGCTGAGCGATTTCTTTA
GAATGCGGAGACAGGAGATCAAGGCCCAGAAGAAGGCACAGAGCTCCACCGACTCTGGAGGATCTAGCGG
AGGATCCTCTGGAAGCGAGACACCAGGCACAAGCGAGTCCGCCACACCAGAGAGCTCCGGCGGCTCCTCC
GGAGGATCCTCTGAGGTGGAGTTTTCCCACGAGTACTGGATGAGACATGCCCTGACCCTGGCCAAGAGGG
CACGCGATGAGAGGGAGGTGCCTGTGGGAGCCGTGCTGGTGCTGAACAATAGAGTGATCGGCGAGGGCTG
GAACAGAGCCATCGGCCTGCACGACCCAACAGCCCATGCCGAAATTATGGCCCTGAGACAGGGCGGCCTG
GTCATGCAGAACTACAGACTGATTGACGCCACCCTGTACGTGACATTCGAGCCTTGCGTGATGTGCGCCG
GCGCCATGATCCACTCTAGGATCGGCCGCGTGGTGTTTGGCGTGAGGAACGCAAAAACCGGCGCCGCAGG
CTCCCTGATGGACGTGCTGCACTACCCCGGCATGAATCACCGCGTCGAAATTACCGAGGGAATCCTGGCA
GATGAATGTGCCGCCCTGCTGTGCTATTTCTTTCGGATGCCTAGACAGGTGTTCAATGCTCAGAAGAAGG
CCCAGAGCTCCACCGACTCCGGAGGATCTAGCGGAGGCTCCTCTGGCTCTGAGACACCTGGCACAAGCGA
GAGCGCAACACCTGAAAGCAGCGGGGGCAGCAGCGGGGGGTCAGACAAGAAGTACAGCATCGGCCTGGCC
ATCGGCACCAACTCTGTGGGCTGGGCCGTGATCACCGACGAGTACAAGGTGCCCAGCAAGAAATTCAAGG
TGCTGGGCAACACCGACCGGCACAGCATCAAGAAGAACCTGATCGGAGCCCTGCTGTTCGACAGCGGCGA
AACAGCCGAGGCCACCCGGCTGAAGAGAACCGCCAGAAGAAGATACACCAGACGGAAGAACCGGATCTGC
TATCTGCAAGAGATCTTCAGCAACGAGATGGCCAAGGTGGACGACAGCTTCTTCCACAGACTGGAAGAGT
CCTTCCTGGTGGAAGAGGATAAGAAGCACGAGCGGCACCCCATCTTCGGCAACATCGTGGACGAGGTGGC
CTACCACGAGAAGTACCCCACCATCTACCACCTGAGAAAGAAACTGGTGGACAGCACCGACAAGGCCGAC
CTGCGGCTGATCTATCTGGCCCTGGCCCACATGATCAAGTTCCGGGGCCACTTCCTGATCGAGGGCGACC
TGAACCCCGACAACAGCGACGTGGACAAGCTGTTCATCCAGCTGGTGCAGACCTACAACCAGCTGTTCGA
GGAAAACCCCATCAACGCCAGCGGCGTGGACGCCAAGGCCATCCTGTCTGCCAGACTGAGCAAGAGCAGA
CGGCTGGAAAATCTGATCGCCCAGCTGCCCGGCGAGAAGAAGAATGGCCTGTTCGGAAACCTGATTGCCC
TGAGCCTGGGCCTGACCCCCAACTTCAAGAGCAACTTCGACCTGGCCGAGGATGCCAAACTGCAGCTGAG
CAAGGACACCTACGACGACGACCTGGACAACCTGCTGGCCCAGATCGGCGACCAGTACGCCGACCTGTTT
CTGGCCGCCAAGAACCTGTCCGACGCCATCCTGCTGAGCGACATCCTGAGAGTGAACACCGAGATCACCA
AGGCCCCCCTGAGCGCCTCTATGATCAAGAGATACGACGAGCACCACCAGGACCTGACCCTGCTGAAAGC
TCTCGTGCGGCAGCAGCTGCCTGAGAAGTACAAAGAGATTTTCTTCGACCAGAGCAAGAACGGCTACGCC
GGCTACATTGACGGCGGAGCCAGCCAGGAAGAGTTCTACAAGTTCATCAAGCCCATCCTGGAAAAGATGG
ACGGCACCGAGGAACTGCTCGTGAAGCTGAACAGAGAGGACCTGCTGCGGAAGCAGCGGACCTTCGACAA
CGGCAGCATCCCCCACCAGATCCACCTGGGAGAGCTGCACGCCATTCTGCGGCGGCAGGAAGATTTTTAC
CCATTCCTGAAGGACAACCGGGAAAAGATCGAGAAGATCCTGACCTTCCGCATCCCCTACTACGTGGGCC
CTCTGGCCAGGGGAAACAGCAGATTCGCCTGGATGACCAGAAAGAGCGAGGAAACCATCACCCCCTGGAA
CTTCGAGGAAGTGGTGGACAAGGGCGCTTCCGCCCAGAGCTTCATCGAGCGGATGACCAACTTCGATAAG
AACCTGCCCAACGAGAAGGTGCTGCCCAAGCACAGCCTGCTGTACGAGTACTTCACCGTGTATAACGAGC
TGACCAAAGTGAAATACGTGACCGAGGGAATGAGAAAGCCCGCCTTCCTGAGCGGCGAGCAGAAAAAGGC
CATCGTGGACCTGCTGTTCAAGACCAACCGGAAAGTGACCGTGAAGCAGCTGAAAGAGGACTACTTCAAG
AAAATCGAGTGCTTCGACTCCGTGGAAATCTCCGGCGTGGAAGATCGGTTCAACGCCTCCCTGGGCACAT
ACCACGATCTGCTGAAAATTATCAAGGACAAGGACTTCCTGGACAATGAGGAAAACGAGGACATTCTGGA
AGATATCGTGCTGACCCTGACACTGTTTGAGGACAGAGAGATGATCGAGGAACGGCTGAAAACCTATGCC
CACCTGTTCGACGACAAAGTGATGAAGCAGCTGAAGCGGCGGAGATACACCGGCTGGGGCAGGCTGAGCC
GGAAGCTGATCAACGGCATCCGGGACAAGCAGTCCGGCAAGACAATCCTGGATTTCCTGAAGTCCGACGG
CTTCGCCAACAGAAACTTCATGCAGCTGATCCACGACGACAGCCTGACCTTTAAAGAGGACATCCAGAAA
GCCCAGGTGTCCGGCCAGGGCGATAGCCTGCACGAGCACATTGCCAATCTGGCCGGCAGCCCCGCCATTA
AGAAGGGCATCCTGCAGACAGTGAAGGTGGTGGACGAGCTCGTGAAAGTGATGGGCCGGCACAAGCCCGA
GAACATCGTGATCGAAATGGCCAGAGAGAACCAGACCACCCAGAAGGGACAGAAGAACAGCCGCGAGAGA
ATGAAGCGGATCGAAGAGGGCATCAAAGAGCTGGGCAGCCAGATCCTGAAAGAACACCCCGTGGAAAACA
CCCAGCTGCAGAACGAGAAGCTGTACCTGTACTACCTGCAGAATGGGCGGGATATGTACGTGGACCAGGA
ACTGGACATCAACCGGCTGTCCGACTACGATGTGGACCATATCGTGCCTCAGAGCTTTCTGAAGGACGAC
TCCATCGACAACAAGGTGCTGACCAGAAGCGACAAGAACCGGGGCAAGAGCGACAACGTGCCCTCCGAAG
AGGTCGTGAAGAAGATGAAGAACTACTGGCGGCAGCTGCTGAACGCCAAGCTGATTACCCAGAGAAAGTT
CGACAATCTGACCAAGGCCGAGAGAGGCGGCCTGAGCGAACTGGATAAGGCCGGCTTCATCAAGAGACAG
CTGGTGGAAACCCGGCAGATCACAAAGCACGTGGCACAGATCCTGGACTCCCGGATGAACACTAAGTACG
ACGAGAATGACAAGCTGATCCGGGAAGTGAAAGTGATCACCCTGAAGTCCAAGCTGGTGTCCGATTTCCG
GAAGGATTTCCAGTTTTACAAAGTGCGCGAGATCAACAACTACCACCACGCCCACGACGCCTACCTGAAC
GCCGTCGTGGGAACCGCCCTGATCAAAAAGTACCCTAAGCTGGAAAGCGAGTTCGTGTACGGCGACTACA
AGGTGTACGACGTGCGGAAGATGATCGCCAAGAGCGAGCAGGAAATCGGCAAGGCTACCGCCAAGTACTT
CTTCTACAGCAACATCATGAACTTTTTCAAGACCGAGATTACCCTGGCCAACGGCGAGATCCGGAAGCGG
CCTCTGATCGAGACAAACGGCGAAACCGGGGAGATCGTGTGGGATAAGGGCCGGGATTTTGCCACCGTGC
GGAAAGTGCTGAGCATGCCCCAAGTGAATATCGTGAAAAAGACCGAGGTGCAGACAGGCGGCTTCAGCAA
AGAGTCTATCCTGCCCAAGAGGAACAGCGATAAGCTGATCGCCAGAAAGAAGGACTGGGACCCTAAGAAG
TACGGCGGCTTCGACAGCCCCACCGTGGCCTATTCTGTGCTGGTGGTGGCCAAAGTGGAAAAGGGCAAGT
CCAAGAAACTGAAGAGTGTGAAAGAGCTGCTGGGGATCACCATCATGGAAAGAAGCAGCTTCGAGAAGAA
TCCCATCGACTTTCTGGAAGCCAAGGGCTACAAAGAAGTGAAAAAGGACCTGATCATCAAGCTGCCTAAG
TACTCCCTGTTCGAGCTGGAAAACGGCCGGAAGAGAATGCTGGCCTCTGCCGGCGAACTGCAGAAGGGAA
ACGAACTGGCCCTGCCCTCCAAATATGTGAACTTCCTGTACCTGGCCAGCCACTATGAGAAGCTGAAGGG
CTCCCCCGAGGATAATGAGCAGAAACAGCTGTTTGTGGAACAGCACAAGCACTACCTGGACGAGATCATC
GAGCAGATCAGCGAGTTCTCCAAGAGAGTGATCCTGGCCGACGCTAATCTGGACAAAGTGCTGTCCGCCT
ACAACAAGCACCGGGATAAGCCCATCAGAGAGCAGGCCGAGAATATCATCCACCTGTTTACCCTGACCAA
TCTGGGAGCCCCTGCCGCCTTCAAGTACTTTGACACCACCATCGACCGGAAGAGGTACACCAGCACCAAA
GAGGTGCTGGACGCCACCCTGATCCACCAGAGCATCACCGGCCTGTACGAGACACGGATCGACCTGTCTC
AGCTGGGAGGTGACTCTGGCGGCTCAAAAAGAACCGCCGACGGCAGCGAATTCGAGCCCAAGAAGAAGAG
GAAAGTCTAACCGGTCATCATCACCATCACCATTGAGTTTAAACCCGCTGATCAGCCTCGACTGTGCCTT
CTAGTTGCCAGCCATCTGTTGTTTGCCCCTCCCCCGTGCCTTCCTTGACCCTGGAAGGTGCCACTCCCAC
TGTCCTTTCCTAATAAAATGAGGAAATTGCATCGCATTGTCTGAGTAGGTGTCATTCTATTCTGGGGGGT
GGGGTGGGGCAGGACAGCAAGGGGGAGGATTGGGAAGACAATAGCAGGCATGCTGGGGATGCGGTGGGCT
CTATGGCTTCTGAGGCGGAAAGAACCAGCTGGGGCTCGATACCGTCGACCTCTAGCTAGAGCTTGGCGTA
ATCATGGTCATAGCTGTTTCCTGTGTGAAATTGTTATCCGCTCACAATTCCACACAACATACGAGCCGGA
AGCATAAAGTGTAAAGCCTAGGGTGCCTAATGAGTGAGCTAACTCACATTAATTGCGTTGCGCTCACTGC
CCGCTTTCCAGTCGGGAAACCTGTCGTGCCAGCTGCATTAATGAATCGGCCAACGCGCGGGGAGAGGCGG
TTTGCGTATTGGGCGCTCTTCCGCTTCCTCGCTCACTGACTCGCTGCGCTCGGTCGTTCGGCTGCGGCGA
GCGGTATCAGCTCACTCAAAGGCGGTAATACGGTTATCCACAGAATCAGGGGATAACGCAGGAAAGAACA
TGTGAGCAAAAGGCCAGCAAAAGGCCAGGAACCGTAAAAAGGCCGCGTTGCTGGCGTTTTTCCATAGGCT
CCGCCCCCCTGACGAGCATCACAAAAATCGACGCTCAAGTCAGAGGTGGCGAAACCCGACAGGACTATAA
AGATACCAGGCGTTTCCCCCTGGAAGCTCCCTCGTGCGCTCTCCTGTTCCGACCCTGCCGCTTACCGGAT
ACCTGTCCGCCTTTCTCCCTTCGGGAAGCGTGGCGCTTTCTCATAGCTCACGCTGTAGGTATCTCAGTTC
GGTGTAGGTCGTTCGCTCCAAGCTGGGCTGTGTGCACGAACCCCCCGTTCAGCCCGACCGCTGCGCCTTA
TCCGGTAACTATCGTCTTGAGTCCAACCCGGTAAGACACGACTTATCGCCACTGGCAGCAGCCACTGGTA
ACAGGATTAGCAGAGCGAGGTATGTAGGCGGTGCTACAGAGTTCTTGAAGTGGTGGCCTAACTACGGCTA
CACTAGAAGAACAGTATTTGGTATCTGCGCTCTGCTGAAGCCAGTTACCTTCGGAAAAAGAGTTGGTAGC
TCTTGATCCGGCAAACAAACCACCGCTGGTAGCGGTGGTTTTTTTGTTTGCAAGCAGCAGATTACGCGCA
GAAAAAAAGGATCTCAAGAAGATCCTTTGATCTTTTCTACGGGGTCTGACACTCAGTGGAACGAAAACTC
ACGTTAAGGGATTTTGGTCATGAGATTATCAAAAAGGATCTTCACCTAGATCCTTTTAAATTAAAAATGA
AGTTTTAAATCAATCTAAAGTATATATGAGTAAACTTGGTCTGACAGTTACCAATGCTTAATCAGTGAGG
CACCTATCTCAGCGATCTGTCTATTTCGTTCATCCATAGTTGCCTGACTCCCCGTCGTGTAGATAACTAC
GATACGGGAGGGCTTACCATCTGGCCCCAGTGCTGCAATGATACCGCGAGACCCACGCTCACCGGCTCCA
GATTTATCAGCAATAAACCAGCCAGCCGGAAGGGCCGAGCGCAGAAGTGGTCCTGCAACTTTATCCGCCT
CCATCCAGTCTATTAATTGTTGCCGGGAAGCTAGAGTAAGTAGTTCGCCAGTTAATAGTTTGCGCAACGT
TGTTGCCATTGCTACAGGCATCGTGGTGTCACGCTCGTCGTTTGGTATGGCTTCATTCAGCTCCGGTTCC
CAACGATCAAGGCGAGTTACATGATCCCCCATGTTGTGCAAAAAAGCGGTTAGCTCCTTCGGTCCTCCGA
TCGTTGTCAGAAGTAAGTTGGCCGCAGTGTTATCACTCATGGTTATGGCAGCACTGCATAATTCTCTTAC
TGTCATGCCATCCGTAAGATGCTTTTCTGTGACTGGTGAGTACTCAACCAAGTCATTCTGAGAATAGTGT
ATGCGGCGACCGAGTTGCTCTTGCCCGGCGTCAATACGGGATAATACCGCGCCACATAGCAGAACTTTAA
AAGTGCTCATCATTGGAAAACGTTCTTCGGGGCGAAAACTCTCAAGGATCTTACCGCTGTTGAGATCCAG
TTCGATGTAACCCACTCGTGCACCCAACTGATCTTCAGCATCTTTTACTTTCACCAGCGTTTCTGGGTGA
GCAAAAACAGGAAGGCAAAATGCCGCAAAAAAGGGAATAAGGGCGACACGGAAATGTTGAATACTCATAC
TCTTCCTTTTTCAATATTATTGAAGCATTTATCAGGGTTATTGTCTCATGAGCGGATACATATTTGAATG
TATTTAGAAAAATAAACAAATAGGGGTTCCGCGCACATTTCCCCGAAAAGTGCCACCTGACGTCGACGGA
TCGGGAGATCGATCTCCCGATCCCCTAGGGTCGACTCTCAGTACAATCTGCTCTGATGCCGCATAGTTAA
GCCAGTATCTGCTCCCTGCTTGTGTGTTGGAGGTCGCTGAGTAGTGCGCGAGCAAAATTTAAGCTACAAC
AAGGCAAGGCTTGACCGACAATTGCATGAAGAATCTGCTTAGGGTTAGGCGTTTTGCGCTGCTTCGCGAT
GTACGGGCCAGATATACGCGTTGACATTGATTATTGACTAGTTATTAATAGTAATCAATTACGGGGTCAT
TAGTTCATAGCCCATATATGGAGTTCCGCGTTACATAACTTACGGTAAATGGCCCGCCTGGCTGACCGCC
CAACGACCCCCGCCCATTGACGTCAATAATGACGTATGTTCCCATAGTAACGCCAATAGGGACTTTCCAT
TGACGTCAATGGGTGGAGTATTTACGGTAAACTGCCCACTTGGCAGTACATCAAGTGTATC

By way of example, a cytidine base editor (CBE) as used in the base editing compositions, systems and methods described herein has the following nucleic acid sequence (8877 base pairs), (Addgene, Watertown, Mass.; Komor A C, et al., 2017, Sci Adv., 30; 3(8):eaao4774. doi: 10.1126/sciadv.aao4774) as provided below. Polynucleotide sequences having at least 95% or greater identity to the BE4 nucleic acid sequence are also encompassed.

| | |
|---|---|
| 1 | ATATGCCAAG TACGCCCCCT ATTGACGTCA ATGACGGTAA ATGGCCCGCC TGGCATTATG |
| 61 | CCCAGTACAT GACCTTATGG GACTTTCCTA CTTGGCAGTA CATCTACGTA TTAGTCATCG |
| 121 | CTATTACCAT GGTGATGCGG TTTTGGCAGT ACATCAATGG GCGTGGATAG CGGTTTGACT |
| 181 | CACGGGGATT TCCAAGTCTC CACCCCATTG ACGTCAATGG GAGTTTGTTT TGGCACCAAA |
| 241 | ATCAACGGGA CTTTCCAAAA TGTCGTAACA ACTCCGCCCC ATTGACGCAA ATGGGCGGTA |
| 301 | GGCGTGTACG GTGGGAGGTC TATATAAGCA GAGCTGGTTT AGTGAACCGT CAGATCCGCT |
| 361 | AGAGATCCGC GGCCGCTAAT ACGACTCACT ATAGGGAGAG CCGCCACCAT GAGCTCAGAG |
| 421 | ACTGGCCCAG TGGCTGTGGA CCCCACATTG AGACGGCGGA TCGAGCCCCA TGAGTTTGAG |
| 481 | GTATTCTTCG ATCCGAGAGA GCTCCGCAAG GAGACCTGCC TGCTTTACGA AATTAATTGG |
| 541 | GGGGGCCGGC ACTCCATTTG GCGACATACA TCACAGAACA CTAACAAGCA CGTCGAAGTC |
| 601 | AACTTCATCG AGAAGTTCAC GACAGAAAGA TATTTCTGTC CGAACACAAG GTGCAGCATT |
| 661 | ACCTGGTTTC TCAGCTGGAG CCCATGCGGC GAATGTAGTA GGGCCATCAC TGAATTCCTG |
| 721 | TCAAGGTATC CCCACGTCAC TCTGTTTATT TACATCGCAA GGCTGTACCA CCACGCTGAC |
| 781 | CCCCGCAATC GACAAGGCCT GCGGGATTTG ATCTCTTCAG GTGTGACTAT CCAAATTATG |
| 841 | ACTGAGCAGG AGTCAGGATA CTGCTGGAGA AACTTTGTGA ATTATAGCCC GAGTAATGAA |
| 901 | GCCCACTGGC CTAGGTATCC CCATCTGTGG GTACGACTGT ACGTTCTTGA ACTGTACTGC |
| 961 | ATCATACTGG GCCTGCCTCC TTGTCTCAAC ATTCTGAGAA GGAAGCAGCC ACAGCTGACA |
| 1021 | TTCTTTACCA TCGCTCTTCA GTCTTGTCAT TACCAGCGAC TGCCCCCACA CATTCTCTGG |
| 1081 | GCCACCGGGT TGAAATCTGG TGGTTCTTCT GGTGGTTCTA GCGGCAGCGA GACTCCCGGG |
| 1141 | ACCTCAGAGT CCGCCACACC CGAAAGTTCT GGTGGTTCTT CTGGTGGTTC TGATAAAAAG |
| 1201 | TATTCTATTG GTTTAGCCAT CGGCACTAAT TCCGTTGGAT GGGCTGTCAT AACCGATGAA |
| 1261 | TACAAAGTAC CTTCAAAGAA ATTTAAGGTG TTGGGGAACA CAGACCGTCA TTCGATTAAA |
| 1321 | AAGAATCTTA TCGGTGCCCT CCTATTCGAT AGTGGCGAAA CGGCAGAGGC GACTCGCCTG |
| 1381 | AAACGAACCG CTCGGAGAAG GTATACACGT CGCAAGAACC GAATATGTTA CTTACAAGAA |
| 1441 | ATTTTTAGCA ATGAGATGGC CAAAGTTGAC GATTCTTTCT TTCACCGTTT GGAAGAGTCC |
| 1501 | TTCCTTGTCG AAGAGGACAA GAAACATGAA CGGCACCCCA TCTTTGGAAA CATAGTAGAT |
| 1561 | GAGGTGGCAT ATCATGAAAA GTACCCAACG ATTTATCACC TCAGAAAAAA GCTAGTTGAC |
| 1621 | TCAACTGATA AAGCGGACCT GAGGTTAATC TACTTGGCTC TTGCCCATAT GATAAAGTTC |
| 1681 | CGTGGGCACT TTCTCATTGA GGGTGATCTA AATCCGGACA ACTCGGATGT CGACAAACTG |
| 1741 | TTCATCCAGT TAGTACAAAC CTATAATCAG TTGTTTGAAG AGAACCCTAT AAATGCAAGT |
| 1801 | GGCGTGGATG CGAAGGCTAT TCTTAGCGCC CGCCTCTCTA AATCCCGACG GCTAGAAAAC |
| 1861 | CTGATCGCAC AATTACCCGG AGAGAAGAAA AATGGGTTGT TCGGTAACCT TATAGCGCTC |
| 1921 | TCACTAGGCC TGACACCAAA TTTTAAGTCG AACTTCGACT TAGCTGAAGA TGCCAAATTG |
| 1981 | CAGCTTAGTA AGGACACGTA CGATGACGAT CTCGACAATC TACTGGCACA AATTGGAGAT |
| 2041 | CAGTATGCGG ACTTATTTTT GGCTGCCAAA AACCTTAGCG ATGCAATCCT CCTATCTGAC |
| 2101 | ATACTGAGAG TTAATACTGA GATTACCAAG GCGCCGTTAT CCGCTTCAAT GATCAAAAGG |
| 2161 | TACGATGAAC ATCACCAAGA CTTGACACTT CTCAAGGCCC TAGTCCGTCA GCAACTGCCT |
| 2221 | GAGAAATATA AGGAAATATT CTTTGATCAG TCGAAAAACG GGTACGCAGG TTATATTGAC |
| 2281 | GGCGGAGCGA GTCAAGAGGA ATTCTACAAG TTTATCAAAC CCATATTAGA GAAGATGGAT |
| 2341 | GGGACGGAAG AGTTGCTTGT AAAACTCAAT CGCGAAGATC TACTGCGAAA GCAGCGGACT |
| 2401 | TTCGACAACG GTAGCATTCC ACATCAAATC CACTTAGGCG AATTGCATGC TATACTTAGA |
| 2461 | AGGCAGGAGG ATTTTTATCC GTTCCTCAAA GACAATCGTG AAAAGATTGA GAAAATCCTA |
| 2521 | ACCTTTCGCA TACCTTACTA TGTGGGACCC CTGGCCCGAG GGAACTCTCG GTTCGCATGG |
| 2581 | ATGACAAGAA AGTCCGAAGA AACGATTACT CCATGGAATT TTGAGGAAGT TGTCGATAAA |
| 2641 | GGTGCGTCAG CTCAATCGTT CATCGAGAGG ATGACCAACT TTGACAAGAA TTTACCGAAC |
| 2701 | GAAAAAGTAT TGCCTAAGCA CAGTTTACTT TACGAGTATT TCACAGTGTA CAATGAACTC |
| 2761 | ACGAAAGTTA AGTATGTCAC TGAGGGCATG CGTAAACCCG CCTTTCTAAG CGGAGAACAG |
| 2821 | AAGAAAGCAA TAGTAGATCT GTTATTCAAG ACCAACCGCA AAGTGACAGT TAAGCAATTG |
| 2881 | AAAGAGGACT ACTTTAAGAA AATTGAATGC TTCGATTCTG TCGAGATCTC CGGGGTAGAA |
| 2941 | GATCGATTTA ATGCGTCACT TGGTACGTAT CATGACCTCC TAAAGATAAT TAAAGATAAG |
| 3001 | GACTTCCTGG ATAACGAAGA GAATGAAGAT ATCTTAGAAG ATATAGTGTT GACTCTTACC |
| 3061 | CTCTTTGAAG ATCGGGAAAT GATTGAGGAA AGACTAAAAA CATACGCTCA CCTGTTCGAC |
| 3121 | GATAAGGTTA TGAAACAGTT AAAGAGGCGT CGCTATACGG GCTGGGGACG ATTGTCGCGG |
| 3181 | AAACTTATCA ACGGGATAAG AGACAAGCAA AGTGGTAAAA CTATTCTCGA TTTTCTAAAG |
| 3241 | AGCGACGGCT TCGCCAATAG GAACTTTATG CAGCTGATCC ATGATGACTC TTTAACCTTC |
| 3301 | AAAGAGGATA TACAAAAGGC ACAGGTTTCC GGACAAGGGG ACTCATTGCA CGAACATATT |
| 3361 | GCGAATCTTG CTGGTTCGCC AGCCATCAAA AAGGGCATAC TCCAGACAGT CAAAGTAGTG |
| 3421 | GATGAGCTAG TTAAGGTCAT GGGACGTCAC AAACCGGAAA ACATTGTAAT CGAGATGGCA |
| 3481 | CGCGAAAATC AAACGACTCA GAAGGGGCAA AAAAACAGTC GAGAGCGGAT GAAGAGAATA |
| 3541 | GAAGAGGGTA TTAAAGAACT GGGCAGCCAG ATCTTAAAGG AGCATCCTGT GGAAAATACC |
| 3601 | CAATTGCAGA ACGAGAAACT TTACCTCTAT TACCTACAAA ATGGAAGGGA CATGTATGTT |
| 3661 | GATCAGGAAC TGGACATAAA CCGTTTATCT GATTACGACG TCGATCACAT TGTACCCCAA |
| 3721 | TCCTTTTTGA AGGACGATTC AATCGACAAT AAAGTGCTTA CACGCTCGGA TAAGAACCGA |
| 3781 | GGGAAAAGTG ACAATGTTCC AAGCGAGGAA GTCGTAAAGA AAATGAAGAA CTATTGGCGG |
| 3841 | CAGCTCCTAA ATGCGAAACT GATAACGCAA AGAAAGTTCG ATAACTTAAC TAAAGCTGAG |
| 3901 | AGGGGTGGCT TGTCTGAACT TGACAAGGCC GGATTTATTA AACGTCAGCT CGTGGAAACC |
| 3961 | CGCCAAATCA CAAAGCATGT TGCACAGATA CTAGATTCCC GAATGAATAC GAAATACGAC |
| 4021 | GAGAACGATA AGCTGATTCG GGAAGTCAAA GTAATCACTT TAAAGTCAAA ATTGGTGTCG |
| 4081 | GACTTCAGAA AGGATTTTCA ATTCTATAAA GTTAGGGAGA TAAATAACTA CCACCATGCG |
| 4141 | CACGACGCTT ATCTTAATGC CGTCGTAGGG ACCGCACTCA TTAAGAAATA CCCGAAGCTA |
| 4201 | GAAAGTGAGT TTGTGTATGG TGATTACAAA GTTTATGACG TCCGTAAGAT GATCGCGAAA |
| 4261 | AGCGAACAGG AGATAGGCAA GGCTACAGCC AAATACTTCT TTTATTCTAA CATTATGAAT |
| 4321 | TTCTTTAAGA CGGAAATCAC TCTGGCAAAC GGAGAGATAC GCAAACGACC TTTAATTGAA |
| 4381 | ACCAATGGGG AGACAGGTGA AATCGTATGG GATAAGGGCC GGGACTTCGC GACGGTGAGA |
| 4441 | AAAGTTTTGT CCATGCCCCA AGTCAACATA GTAAAGAAAA CTGAGGTGCA GACCGGAGGG |
| 4501 | TTTTCAAAGG AATCGATTCT TCCAAAAAGG AATAGTGATA AGCTCATCGC TCGTAAAAAG |
| 4561 | GACTGGGACC CGAAAAAGTA CGGTGGCTTC GATAGCCCTA CAGTTGCCTA TTCTGTCCTA |
| 4621 | GTAGTGGCAA AAGTTGAGAA GGGAAAATCC AAGAAACTGA AGTCAGTCAA AGAATTATTG |
| 4681 | GGGATAACGA TTATGGAGCG CTCGTCTTTT GAAAAGAACC CCATCGACTT CCTTGAGGCG |
| 4741 | AAAGGTTACA AGGAAGTAAA AAAGGATCTC ATAATTAAAC TACCAAAGTA TAGTCTGTTT |
| 4801 | GAGTTAGAAA ATGGCCGAAA ACGGATGTTG GCTAGCGCCG GAGAGCTTCA AAAGGGGAAC |
| 4861 | GAACTCGCAC TACCGTCTAA ATACGTGAAT TTCCTGTATT TAGCGTCCCA TTACGAGAAG |
| 4921 | TTGAAAGGTT CACCTGAAGA TAACGAACAG AAGCAACTTT TTGTTGAGCA GCACAAACAT |
| 4981 | TATCTCGACG AAATCATAGA GCAAATTTCG GAATTCAGTA AGAGAGTCAT CCTAGCTGAT |
| 5041 | GCCAATCTGG ACAAAGTATT AAGCGCATAC AACAAGCACA GGGATAAACC CATACGTGAG |
| 5101 | CAGGCGGAAA ATATTATCCA TTTGTTTACT CTTACCAACC TCGGCGCTCC AGCCGCATTC |
| 5161 | AAGTATTTTG ACACAACGAT AGATCGCAAA CGATACACTT CTACCAAGGA GGTGCTAGAC |
| 5221 | GCGACACTGA TTCACCAATC CATCACGGGA TTATATGAAA CTCGGATAGA TTTGTCACAG |
| 5281 | CTTGGGGGTG ACTCTGGTGG TTCTGGAGGA TCTGGTGGTT CTACTAATCT GTCAGATATT |
| 5341 | ATTGAAAAGG AGACCGGTAA GCAACTGGTT ATCCAGGAAT CCATCCTCAT GCTCCCAGAG |
| 5401 | GAGGTGGAAG AAGTCATTGG GAACAAGCCG GAAAGCGATA TACTCGTGCA CACCGCCTAC |
| 5461 | GACGAGAGCA CCGACGAGAA TGTCATGCTT CTGACTAGCG ACGCCCCTGA ATACAAGCCT |
| 5521 | TGGGCTCTGG TCATACAGGA TAGCAACGGT GAGAACAAGA TTAAGATGCT CTCTGGTGGT |
| 5581 | TCTGGAGGAT CTGGTGGTTC TACTAATCTG TCAGATATTA TTGAAAAGGA GACCGGTAAG |
| 5641 | CAACTGGTTA TCCAGGAATC CATCCTCATG CTCCCAGAGG AGGTGGAAGA AGTCATTGGG |
| 5701 | AACAAGCCGG AAAGCGATAT ACTCGTGCAC ACCGCCTACG ACGAGAGCAC CGACGAGAAT |
| 5761 | GTCATGCTTC TGACTAGCGA CGCCCCTGAA TACAAGCCTT GGGCTCTGGT CATACAGGAT |
| 5821 | AGCAACGGTG AGAACAAGAT TAAGATGCTC TCTGGTGGTT CTCCCAAGAA GAAGAGGAAA |
| 5881 | GTCTAACCGG TCATCATCAC CATCACCATT GAGTTTAAAC CCGCTGATCA GCCTCGACTG |
| 5941 | TGCCTTCTAG TTGCCAGCCA TCTGTTGTTT GCCCCTCCCC CGTGCCTTCC TTGACCCTGG |
| 6001 | AAGGTGCCAC TCCCACTGTC CTTTCCTAAT AAAATGAGGA AATTGCATCG CATTGTCTGA |
| 6061 | GTAGGTGTCA TTCTATTCTG GGGGGTGGGG TGGGGCAGGA CAGCAAGGGG GAGGATTGGG |
| 6121 | AAGACAATAG CAGGCATGCT GGGGATGCGG TGGGCTCTAT GGCTTCTGAG GCGGAAAGAA |
| 6181 | CCAGCTGGGG CTCGATACCG TCGACCTCTA GCTAGAGCTT GGCGTAATCA TGGTCATAGC |
| 6241 | TGTTTCCTGT GTGAAATTGT TATCCGCTCA CAATTCCACA CAACATACGA GCCGGAAGCA |
| 6301 | TAAAGTGTAA AGCCTAGGGT GCCTAATGAG TGAGCTAACT CACATTAATT GCGTTGCGCT |
| 6361 | CACTGCCCGC TTTCCAGTCG GGAAACCTGT CGTGCCAGCT GCATTAATGA ATCGGCCAAC |
| 6421 | GCGCGGGGAG AGGCGGTTTG CGTATTGGGC GCTCTTCCGC TTCCTCGCTC ACTGACTCGC |
| 6481 | TGCGCTCGGT CGTTCGGCTG CGGCGAGCGG TATCAGCTCA CTCAAAGGCG GTAATACGGT |
| 6541 | TATCCACAGA ATCAGGGGAT AACGCAGGAA AGAACATGTG AGCAAAAGGC CAGCAAAAGG |
| 6601 | CCAGGAACCG TAAAAAGGCC GCGTTGCTGG CGTTTTTCCA TAGGCTCCGC CCCCCTGACG |
| 6661 | AGCATCACAA AAATCGACGC TCAAGTCAGA GGTGGCGAAA CCCGACAGGA CTATAAAGAT |
| 6721 | ACCAGGCGTT TCCCCCTGGA AGCTCCCTCG TGCGCTCTCC TGTTCCGACC CTGCCGCTTA |
| 6781 | CCGGATACCT GTCCGCCTTT CTCCCTTCGG GAAGCGTGGC GCTTTCTCAT AGCTCACGCT |
| 6841 | GTAGGTATCT CAGTTCGGTG TAGGTCGTTC GCTCCAAGCT GGGCTGTGTG CACGAACCCC |
| 6901 | CCGTTCAGCC CGACCGCTGC GCCTTATCCG GTAACTATCG TCTTGAGTCC AACCCGGTAA |
| 6961 | GACACGACTT ATCGCCACTG GCAGCAGCCA CTGGTAACAG GATTAGCAGA GCGAGGTATG |
| 7021 | TAGGCGGTGC TACAGAGTTC TTGAAGTGGT GGCCTAACTA CGGCTACACT AGAAGAACAG |
| 7081 | TATTTGGTAT CTGCGCTCTG CTGAAGCCAG TTACCTTCGG AAAAAGAGTT GGTAGCTCTT |
| 7141 | GATCCGGCAA ACAAACCACC GCTGGTAGCG GTGGTTTTTT TGTTTGCAAG CAGCAGATTA |
| 7201 | CGCGCAGAAA AAAAGGATCT CAAGAAGATC CTTTGATCTT TTCTACGGGG TCTGACGCTC |
| 7261 | AGTGGAACGA AAACTCACGT TAAGGGATTT TGGTCATGAG ATTATCAAAA AGGATCTTCA |
| 7321 | CCTAGATCCT TTTAAATTAA AAATGAAGTT TTAAATCAAT CTAAAGTATA TATGAGTAAA |
| 7381 | CTTGGTCTGA CAGTTACCAA TGCTTAATCA GTGAGGCACC TATCTCAGCG ATCTGTCTAT |
| 7441 | TTCGTTCATC CATAGTTGCC TGACTCCCCG TCGTGTAGAT AACTACGATA CGGGAGGGCT |
| 7501 | TACCATCTGG CCCCAGTGCT GCAATGATAC CGCGAGACCC ACGCTCACCG GCTCCAGATT |
| 7561 | TATCAGCAAT AAACCAGCCA GCCGGAAGGG CCGAGCGCAG AAGTGGTCCT GCAACTTTAT |
| 7621 | CCGCCTCCAT CCAGTCTATT AATTGTTGCC GGGAAGCTAG AGTAAGTAGT TCGCCAGTTA |
| 7681 | ATAGTTTGCG CAACGTTGTT GCCATTGCTA CAGGCATCGT GGTGTCACGC TCGTCGTTTG |
| 7741 | GTATGGCTTC ATTCAGCTCC GGTTCCCAAC GATCAAGGCG AGTTACATGA TCCCCCATGT |
| 7801 | TGTGCAAAAA AGCGGTTAGC TCCTTCGGTC CTCCGATCGT TGTCAGAAGT AAGTTGGCCG |
| 7861 | CAGTGTTATC ACTCATGGTT ATGGCAGCAC TGCATAATTC TCTTACTGTC ATGCCATCCG |
| 7921 | TAAGATGCTT TTCTGTGACT GGTGAGTACT CAACCAAGTC ATTCTGAGAA TAGTGTATGC |
| 7981 | GGCGACCGAG TTGCTCTTGC CCGGCGTCAA TACGGGATAA TACCGCGCCA CATAGCAGAA |
| 8041 | CTTTAAAAGT GCTCATCATT GGAAAACGTT CTTCGGGGCG AAAACTCTCA AGGATCTTAC |
| 8101 | CGCTGTTGAG ATCCAGTTCG ATGTAACCCA CTCGTGCACC CAACTGATCT TCAGCATCTT |
| 8161 | TTACTTTCAC CAGCGTTTCT GGGTGAGCAA AAACAGGAAG GCAAAATGCC GCAAAAAAGG |
| 8221 | GAATAAGGGC GACACGGAAA TGTTGAATAC TCATACTCTT CCTTTTTCAA TATTATTGAA |
| 8281 | GCATTTATCA GGGTTATTGT CTCATGAGCG GATACATATT TGAATGTATT TAGAAAAATA |
| 8341 | AACAAATAGG GGTTCCGCGC ACATTTCCCC GAAAAGTGCC ACCTGACGTC GACGGATCGG |
| 8401 | GAGATCGATC TCCCGATCCC CTAGGGTCGA CTCTCAGTAC AATCTGCTCT GATGCCGCAT |
| 8461 | AGTTAAGCCA GTATCTGCTC CCTGCTTGTG TGTTGGAGGT CGCTGAGTAG TGCGCGAGCA |
| 8521 | AAATTTAAGC TACAACAAGG CAAGGCTTGA CCGACAATTG CATGAAGAAT CTGCTTAGGG |
| 8581 | TTAGGCGTTT TGCGCTGCTT CGCGATGTAC GGGCCAGATA TACGCGTTGA CATTGATTAT |
| 8641 | TGACTAGTTA TTAATAGTAA TCAATTACGG GGTCATTAGT TCATAGCCCA TATATGGAGT |
| 8701 | TCCGCGTTAC ATAACTTACG GTAAATGGCC CGCCTGGCTG ACCGCCCAAC GACCCCCGCC |
| 8761 | CATTGACGTC AATAATGACG TATGTTCCCA TAGTAACGCC AATAGGGACT TTCCATTGAC |
| 8821 | GTCAATGGGT GGAGTATTTA CGGTAAACTG CCCACTTGGC AGTACATCAA GTGTATC |

In some embodiments, the cytidine base editor is BE4 having a nucleic acid sequence selected from one of the following:

Original BE4 nucleic acid sequence:
ATGagctcagagactggcccagtggctgtggaccccacattgagacggcggatcgagccccatgagtt
tgaggtattcttcgatccgagagagctccgcaaggagacctgcctgctttacgaaattaattgggggg
gccggcactccatttggcgacatacatcacagaacactaacaagcacgtcgaagtcaacttcatcgag
aagttcacgacagaaagatatttctgtccgaacacaaggtgcagcattacctggtttctcagctggag
ccgcgaatgtagtagggccatcactgaattcctgtcaaggtatccccacgtcactctgtttatttaca
tcgcaaggctgtaccaccacgctgacccccgcaatcgacaaggcctgcgggatttgatctcttcaggt
gtgactatccaaattatgactgagcaggagtcaggatactgctggagaaactttgtgaattatagccc
gagtaatgaagcccactggcctaggtatccccatctgtgggtacgactgtacgttcttgaactgtact
gcatcatactgggcctgcctccttgtctcaacattctgagaaggaagcagccacagctgacattcttt
accatcgctcttcagtcttgtcattaccagcgactgcccccacacattctctgggccaccgggttgaa
atctggtggttcttctggtggttctagcggcagcgagactcccgggacctcagagtccgccacacccg
aaagttctggtggttcttctggtggttctgataaaaagtattctattggtttagccatcggcactaat
tccgttggatgggctgtcataaccgatgaatacaaagtaccttcaaagaaatttaaggtgttggggaa
cacagaccgtcattcgattaaaaagaatcttatcggtgccctcctattcgatagtggcgaaacggcag
aggcgactcgcctgaaacgaaccgctcggagaaggtatacacgtcgcaagaaccgaatatgttactta
caagaaatttttagcaatgagatggccaaagttgacgattctttctttcaccgtttggaagagtcctt
ccttgtcgaagaggacaagaaacatgaacggcaccccatctttggaaacatagtagatgaggtggcat
atcatgaaaagtacccaacgatttatcacctcagaaaaaagctagttgactcaactgataaagcggac
ctgaggttaatctacttggctcttgcccatatgataaagttccgtgggcactttctcattgagggtga
tctaaatccggacaactcggatgtcgacaaactgttcatccagttagtacaaacctataatcagttgt
ttgaagagaaccctataaatgcaagtggcgtggatgcgaaggctattcttagcgcccgcctctctaaa
tcccgacggctagaaaacctgatcgcacaattacccggagagaagaaaaatgggttgttcggtaacct
tatagcgctctcactaggcctgacaccaaattttaagtcgaacttcgacttagctgaagatgccaaat
tgcagcttagtaaggacacgtacgatgacgatctcgacaatctactggcacaaattggagatcagtat
gcggacttatttttggctgccaaaaaccttagcgatgcaatcctcctatctgacatactgagagttaa
tactgagattaccaaggcgccgttatccgcttcaatgatcaaaaggtacgatgaacatcaccaagact
tgacacttctcaaggccctagtccgtcagcaactgcctgagaaatataaggaaatattctttgatcag
tcgaaaaacgggtacgcaggttatattgacggcggagcgagtcaagaggaattctacaagtttatcaa
acccatattagagaagatggatgggacggaagagttgcttgtaaaactcaatcgcgaagatctactgc
gaaagcagcggactttcgacaacggtagcattccacatcaaatccacttaggcgaattgcatgctata
cttagaaggcaggaggatttttatccgttcctcaaagacaatcgtgaaaagattgagaaaatcctaac
ctttcgcataccttactatgtgggacccctggcccgagggaactctcggttcgcatggatgacaagaa
agtccgaagaaacgattactccatggaattttgaggaagttgtcgataaaggtgcgtcagctcaatcg
ttcatcgagaggatgaccaactttgacaagaatttaccgaacgaaaaagtattgcctaagcacagttt
actttacgagtatttcacagtgtacaatgaactcacgaaagttaagtatgtcactgagggcatgcgta
aacccgcctttctaagcggagaacagaagaaagcaatagtagatctgttattcaagaccaaccgcaaa
gtgacagttaagcaattgaaagaggactactttaagaaaattgaatgcttcgattctgtcgagatctc
cggggtagaagatcgatttaatgcgtcacttggtacgtatcatgacctcctaaagataattaaagata
aggacttcctggataacgaagagaatgaagatatcttagaagatatagtgttgactcttaccctcttt
gaagatcgggaaatgattgaggaaagactaaaaacatacgctcacctgttcgacgataaggttatgaa
acagttaaagaggcgtcgctatacgggctggggacgattgtcgcggaaacttatcaacgggataagag
acaagcaaagtggtaaaactattctcgattttctaaagagcgacggcttcgccaataggaactttatg
cagctgatccatgatgactctttaaccttcaaagaggatatacaaaaggcacaggtttccggacaagg
ggactcattgcacgaacatattgcgaatcttgctggttcgccagccatcaaaaagggcatactccaga
cagtcaaagtagtggatgagctagttaaggtcatgggacgtcacaaaccggaaaacattgtaatcgag
atggcacgcgaaaatcaaacgactcagaaggggcaaaaaaacagtcgagagcggatgaagagaataga
agagggtattaaagaactgggcagccagatcttaaaggagcatcctgtggaaaatacccaattgcaga
acgagaaactttacctctattacctacaaaatggaagggacatgtatgttgatcaggaactggacata
aaccgtttatctgattacgacgtcgatcacattgtaccccaatcctttttgaaggacgattcaatcga
caataaagtgcttacacgctcggataagaaccgagggaaaagtgacaatgttccaagcgaggaagtcg
taaagaaaatgaagaactattggcggcagctcctaaatgcgaaactgataacgcaaagaaagttcgat
aacttaactaaagctgagaggggtggcttgtctgaacttgacaaggccggatttattaaacgtcagct
cgtggaaacccgccaaatcacaaagcatgttgcacagatactagattcccgaatgaatacgaaatacg
acgagaacgataagctgattcgggaagtcaaagtaatcactttaaagtcaaaattggtgtcggacttc
agaaaggattttcaattctataaagttagggagataaataactaccaccatgcgcacgacgcttatct
taatgccgtcgtagggaccgcactcattaagaaatacccgaagctagaaagtgagtttgtgtatggtg
attacaaagtttatgacgtccgtaagatgatcgcgaaaagcgaacaggagataggcaaggctacagcc
aaatacttcttttattctaacattatgaatttctttaagacggaaatcactctggcaaacggagagat
acgcaaacgacctttaattgaaaccaatggggagacaggtgaaatcgtatgggataagggccgggact
tcgcgacggtgagaaaagttttgtccatgccccaagtcaacatagtaaagaaaactgaggtgcagacc
ggagggttttcaaaggaatcgattcttccaaaaaggaatagtgataagctcatcgctcgtaaaaagga
ctgggacccgaaaaagtacggtggcttcgatagccctacagttgcctattctgtcctagtagtggcaa
aagttgagaagggaaaatccaagaaactgaagtcagtcaaagaattattggggataacgattatggag
cgctcgtcttttgaaaagaaccccatcgacttccttgaggcgaaaggttacaaggaagtaaaaaagga
tctcataattaaactaccaaagtatagtctgtttgagttagaaaatggccgaaaacggatgttggcta
gcgccggagagcttcaaaaggggaacgaactcgcactaccgtctaaatacgtgaatttcctgtattta
gcgtcccattacgagaagttgaaaggttcacctgaagataacgaacagaagcaactttttgttgagca
gcacaaacattatctcgacgaaatcatagagcaaatttcggaattcagtaagagagtcatcctagctg
atgccaatctggacaaagtattaagcgcatacaacaagcacagggataaacccatacgtgagcaggcg
gaaaatattatccatttgtttactcttaccaacctcggcgctccagccgcattcaagtattttgacac
aacgatagatcgcaaacgatacacttctaccaaggaggtgctagacgcgacactgattcaccaatcca
tcacgggattatatgaaactcggatagatttgtcacagcttgggggtgactctggtggttctggagga
tctggtggttctactaatctgtcagatattattgaaaaggagaccggtaagcaactggttatccagga
atccatcctcatgctcccagaggaggtggaagaagtcattgggaacaagccggaaagcgatatactcg
tgcacaccgcctacgacgagagcaccgacgagaatgtcatgcttctgactagcgacgcccctgaatac
aagccttgggctctggtcatacaggatagcaacggtgagaacaagattaagatgctctctggtggttc
tggaggatctggtggttctactaatctgtcagatattattgaaaaggagaccggtaagcaactggtta
tccaggaatccatcctcatgctcccagaggaggtggaagaagtcattgggaacaagccggaaagcgat
atactcgtgcacaccgcctacgacgagagcaccgacgagaatgtcatgcttctgactagcgacgcccc
tgaatacaagccttgggctctggtcatacaggatagcaacggtgagaacaagattaagatgctctctg
gtggttctAAAAGGACGGCGGACGGATCAGAGTTCGAGAGTCCGAAAAAAAAACGAAAGGTCGAAtaa
BE4 Codon Optimization 1 nucleic acid sequence:
ATGTCATCCGAAACCGGGCCAGTGGCCGTAGACCCAACACTCAGGAGGCGGATAGAACCCCATGAGTT
TGAAGTGTTCTTCGACCCCAGAGAGCTGCGCAAAGAGACTTGCCTCCTGTATGAAATAAATTGGGGGG
GTCGCCATTCAATTTGGAGGCACACTAGCCAGAATACTAACAAACACGTGGAGGTAAATTTTATCGAG
AAGTTTACCACCGAAAGATACTTTTGCCCCAATACACGGTGTTCAATTACCTGGTTTCTGTCATGGAG
TCCATGTGGAGAATGTAGTAGAGCGATAACTGAGTTCCTGTCTCGATATCCTCACGTCACGTTGTTTA
TATACATCGCTCGGCTTTATCACCATGCGGACCCGCGGAACAGGCAAGGTCTTCGGGACCTCATATCC
TCTGGGGTGACCATCCAGATAATGACGGAGCAAGAGAGCGGATACTGCTGGCGAAACTTTGTTAACTA
CAGCCCAAGCAATGAGGCACACTGGCCTAGATATCCGCATCTCTGGGTTCGACTGTATGTCCTTGAAC
TGTACTGCATAATTCTGGGACTTCCGCCATGCTTGAACATTCTGCGGCGGAAACAACCACAGCTGACC
TTTTTCACGATTGCTCTCCAAAGTTGTCACTACCAGCGATTGCCACCCCACATCTTGTGGGCTACTGG
ACTCAAGTCTGGAGGAAGTTCAGGCGGAAGCAGCGGGTCTGAAACGCCCGGAACCTCAGAGAGCGCAA
CGCCCGAAAGCTCTGGAGGGTCAAGTGGTGGTAGTGATAAGAAATACTCCATCGGCCTCGCCATCGGT
ACGAATTCTGTCGGTTGGGCCGTTATCACCGATGAGTACAAGGTCCCTTCTAAGAAATTCAAGGTTTT
GGGCAATACAGACCGCCATTCTATAAAAAAAAACCTGATCGGCGCCCTTTTGTTTGACAGTGGTGAGA
CTGCTGAAGCGACTCGCCTGAAGCGAACTGCCAGGAGGCGGTATACGAGGCGAAAAAACCGAATTTGT
TACCTCCAGGAGATTTTCTCAAATGAAATGGCCAAGGTAGATGATAGTTTTTTTCACCGCTTGGAAGA
AAGTTTTCTCGTTGAGGAGGACAAAAAGCACGAGAGGCACCCAATCTTTGGCAACATAGTCGATGAGG
TCGCATACCATGAGAAATATCCTACGATCTATCATCTCCGCAAGAAGCTGGTCGATAGCACGGATAAA
GCTGACCTCCGGCTGATCTACCTTGCTCTTGCTCACATGATTAAATTCAGGGGCCATTTCCTGATAGA
AGGAGACCTCAATCCCGACAATTCTGATGTCGACAAACTGTTTATTCAGCTCGTTCAGACCTATAATC
AACTCTTTGAGGAGAACCCCATCAATGCTTCAGGGGTGGACGCAAAGGCCATTTTGTCCGCGCGCTTG
AGTAAATCACGACGCCTCGAGAATTTGATAGCTCAACTGCCGGGTGAGAAGAAAAACGGGTTGTTTGG
GAATCTCATAGCGTTGAGTTTGGGACTTACGCCAAACTTTAAGTCTAACTTTGATTTGGCCGAAGATG
CCAAATTGCAGCTGTCCAAAGATACCTATGATGACGACTTGGATAACCTTCTTGCGCAGATTGGTGAC
CAATACGCGGATCTGTTTCTTGCCGCAAAAAATCTGTCCGACGCCATACTCTTGTCCGATATACTGCG
CGTCAATACTGAGATAACTAAGGCTCCCCTCAGCGCGTCCATGATTAAAAGATACGATGAGCACCACC
AAGATCTCACTCTGTTGAAAGCCCTGGTTCGCCAGCAGCTTCCAGAGAAGTATAAGGAGATATTTTTC
GACCAATCTAAAAACGGCTATGCGGGTTACATTGACGGTGGCGCCTCTCAAGAAGAATTCTACAAGTT
TATAAAGCCGATACTTGAGAAAATGGACGGTACAGAGGAATTGTTGGTTAAGCTCAATCGCGAGGACT
TGTTGAGAAAGCAGCGCACATTTGACAATGGTAGTATTCCACACCAGATTCATCTGGGCGAGTTGCAT
GCCATTCTTAGAAGACAAGAAGATTTTTATCCGTTTCTGAAAGATAACAGAGAAAAGATTGAAAAGAT
ACTTACCTTTCGCATACCGTATTATGTAGGTCCCCTGGCTAGAGGGAACAGTCGCTTCGCTTGGATGA
CTCGAAAATCAGAAGAAACAATAACCCCCTGGAATTTTGAAGAAGTGGTAGATAAAGGTGCGAGTGCC
CAATCTTTTATTGAGCGGATGACAAATTTTGACAAGAATCTGCCTAACGAAAAGGTGCTTCCCAAGCA
TTCCCTTTTGTATGAATACTTTACAGTATATAATGAACTGACTAAAGTGAAGTACGTTACCGAGGGGA
TGCGAAAGCCAGCTTTTCTCAGTGGCGAGCAGAAAAAAGCAATAGTTGACCTGCTGTTCAAGACGAAT
AGGAAGGTTACCGTCAAACAGCTCAAAGAAGATTACTTTAAAAAGATCGAATGTTTTGATTCAGTTGA
GATAAGCGGAGTAGAGGATAGATTTAACGCAAGTCTTGGAACTTATCATGACCTTTTGAAGATCATCA
AGGATAAAGATTTTTTGGACAACGAGGAGAATGAAGATATCCTGGAAGATATAGTACTTACCTTGACG
CTTTTTGAAGATCGAGAGATGATCGAGGAGCGACTTAAGACGTACGCACATCTCTTTGACGATAAGGT
TATGAAACAATTGAAACGCCGGCGGTATACTGGCTGGGGCAGGCTTTCTCGAAAGCTGATTAATGGTA
TCCGCGATAAGCAGTCTGGAAAGACAATCCTTGACTTTCTGAAAAGTGATGGATTTGCAAATAGAAAC
TTTATGCAGCTTATACATGATGACTCTTTGACGTTCAAGGAAGACATCCAGAAGGCACAGGTATCCGG
CCAAGGGGATAGCCTCCATGAACACATAGCCAACCTGGCCGGCTCACCAGCTATTAAAAAGGGAATAT
TGCAAACCGTTAAGGTTGTTGACGAACTCGTTAAGGTTATGGGCCGACACAAACCAGAGAATATCGTG
ATTGAGATGGCTAGGGAGAATCAGACCACTCAAAAAGGTCAGAAAAATTCTCGCGAAAGGATGAAGCG
AATTGAAGAGGGAATCAAAGAACTTGGCTCTCAAATTTTGAAAGAGCACCCGGTAGAAAACACTCAGC
TGCAGAATGAAAAGCTGTATCTGTATTATCTGCAGAATGGTCGAGATATGTACGTTGATCAGGAGCTG
GATATCAATAGGCTCAGTGACTACGATGTCGACCACATCGTTCCTCAATCTTTCCTGAAAGATGACTC
TATCGACAACAAAGTGTTGACGCGATCAGATAAGAACCGGGGAAAATCCGACAATGTACCCTCAGAAG
AAGTTGTCAAGAAGATGAAAAACTATTGGAGACAATTGCTGAACGCCAAGCTCATAACACAACGCAAG
TTCGATAACTTGACGAAAGCCGAAAGAGGTGGGTTGTCAGAATTGGACAAAGCTGGCTTTATTAAGCG
CCAATTGGTGGAGACCCGGCAGATTACGAAACACGTAGCACAAATTTTGGATTCACGAATGAATACCA
AATACGACGAAAACGACAAATTGATACGCGAGGTGAAAGTGATTACGCTTAAGAGTAAGTTGGTTTCC
GATTTCAGGAAGGATTTTCAGTTTTACAAAGTAAGAGAAATAAACAACTACCACCACGCCCATGATGC
TTACCTCAACGCGGTAGTTGGCACAGCTCTTATCAAAAAATATCCAAAGCTGGAAAGCGAGTTCGTTT
ACGGTGACTATAAAGTATACGACGTTCGGAAGATGATAGCCAAATCAGAGCAGGAAATTGGGAAGGCA
ACCGCAAAATACTTCTTCTATTCAAACATCATGAACTTCTTTAAGACGGAGATTACGCTCGCGAACGG
CGAAATACGCAAGAGGCCCCTCATAGAGACTAACGGCGAAACCGGGGAGATCGTATGGGACAAAGGAC
GGGACTTTGCGACCGTTAGAAAAGTACTTTCAATGCCACAAGTGAATATTGTTAAAAAGACAGAAGTA
CAAACAGGGGGGTTCAGTAAGGAATCCATTTTGCCCAAGCGGAACAGTGATAAATTGATAGCAAGGAA
AAAAGATTGGGACCCTAAGAAGTACGGTGGTTTCGACTCTCCTACCGTTGCATATTCAGTCCTTGTAG
TTGCGAAAGTGGAAAAGGGGAAAAGTAAGAAGCTTAAGAGTGTTAAAGAGCTTCTGGGCATAACCATA
ATGGAACGGTCTAGCTTCGAGAAAAATCCAATTGACTTTCTCGAGGCTAAAGGTTACAAGGAGGTAAA
AAAGGACCTGATAATTAAACTCCCAAAGTACAGTCTCTTCGAGTTGGAGAATGGGAGGAAGAGAATGT
TGGCATCTGCAGGGGAGCTCCAAAAGGGGAACGAGCTGGCTCTGCCTTCAAAATACGTGAACTTTCTG
TACCTGGCCAGCCACTACGAGAAACTCAAGGGTTCTCCTGAGGATAACGAGCAGAAACAGCTGTTTGT
AGAGCAGCACAAGCATTACCTGGACGAGATAATTGAGCAAATTAGTGAGTTCTCAAAAAGAGTAATCC
TTGCAGACGCGAATCTGGATAAAGTTCTTTCCGCCTATAATAAGCACCGGGACAAGCCTATACGAGAA
CAAGCCGAGAACATCATTCACCTCTTTACCCTTACTAATCTGGGCGCGCCGGCCGCCTTCAAATACTT
CGACACCACGATAGACAGGAAAAGGTATACGAGTACCAAAGAAGTACTTGACGCCACTCTCATCCACC
AGTCTATAACAGGGTTGTACGAAACGAGGATAGATTTGTCCCAGCTCGGCGGCGACTCAGGAGGGTCA
GGCGGCTCCGGTGGATCAACGAATCTTTCCGACATAATCGAGAAAGAAACCGGCAAACAGTTGGTGAT
CCAAGAATCAATCCTGATGCTGCCTGAAGAAGTAGAAGAGGTGATTGGCAACAAACCTGAGTCTGACA
TTCTTGTCCACACCGCGTATGACGAGAGCACGGACGAGAACGTTATGCTTCTCACTAGCGACGCCCCT
GAGTATAAACCATGGGCGCTGGTCATCCAAGATTCCAATGGGGAAAACAAGATTAAGATGCTTAGTGG
TGGGTCTGGAGGGAGCGGTGGGTCCACGAACCTCAGCGACATTATTGAAAAAGAGACTGGTAAACAAC
TTGTAATACAAGAGTCTATTCTGATGTTGCCTGAAGAGGTGGAGGAGGTGATTGGGAACAAACCGGAG
TCTGATATACTTGTTCATACCGCCTATGACGAATCTACTGATGAGAATGTGATGCTTTTaACGTCAGA
CGCTCCCGAGTACAAACCCTGGGCTCTGGTGATTCAGGACAGCAATGGTGAGAATAAGATTAAAATGT
TGAGTGGGGGCTCAAAGCGCACGGCTGACGGTAGCGAATTTGAGAGCCCCAAAAAAAAACGAAAGGTC
GAAtaa
BE4 Codon Optimization 2 nucleic acid sequence:
ATGAGCAGCGAGACAGGCCCTGTGGCTGTGGATCCTACACTGCGGAGAAGAATCGAGCCCCACGAGTT
CGAGGTGTTCTTCGACCCCAGAGAGCTGCGGAAAGAGACATGCCTGCTGTACGAGATCAACTGGGGCG
GCAGACACTCTATCTGGCGGCACACAAGCCAGAACACCAACAAGCACGTGGAAGTGAACTTTATCGAG
AAGTTTACGACCGAGCGGTACTTCTGCCCCAACACCAGATGCAGCATCACCTGGTTTCTGAGCTGGTC
CCCTTGCGGCGAGTGCAGCAGAGCCATCACCGAGTTTCTGTCCAGATATCCCCACGTGACCCTGTTCA
TCTATATCGCCCGGCTGTACCACCACGCCGATCCTAGAAATAGACAGGGACTGCGCGACCTGATCAGC
AGCGGAGTGACCATCCAGATCATGACCGAGCAAGAGAGCGGCTACTGCTGGCGGAACTTCGTGAACTA
CAGCCCCAGCAACGAAGCCCACTGGCCTAGATATCCTCACCTGTGGGTCCGACTGTACGTGCTGGAAC
TGTACTGCATCATCCTGGGCCTGCCTCCATGCCTGAACATCCTGAGAAGAAAGCAGCCTCAGCTGACC
TTCTTCACAATCGCCCTGCAGAGCTGCCACTACCAGAGACTGCCTCCACACATCCTGTGGGCCACCGG
ACTTAAGAGCGGAGGATCTAGCGGCGGCTCTAGCGGATCTGAGACACCTGGCACAAGCGAGTCTGCCA
CACCTGAGAGTAGCGGCGGATCTTCTGGCGGCTCCGACAAGAAGTACTCTATCGGACTGGCCATCGGC
ACCAACTCTGTTGGATGGGCCGTGATCACCGACGAGTACAAGGTGCCCAGCAAGAAATTCAAGGTGCT
GGGCAACACCGACCGGCACAGCATCAAGAAGAATCTGATCGGCGCCCTGCTGTTCGACTCTGGCGAAA
CAGCCGAAGCCACCAGACTGAAGAGAACCGCCAGGCGGAGATACACCCGGCGGAAGAACCGGATCTGC
TACCTGCAAGAGATCTTCAGCAACGAGATGGCCAAGGTGGACGACAGCTTCTTCCACAGACTGGAAGA
GTCCTTCCTGGTGGAAGAGGACAAGAAGCACGAGCGGCACCCCATCTTCGGCAACATCGTGGATGAGG
TGGCCTACCACGAGAAGTACCCCACCATCTACCACCTGAGAAAGAAACTGGTGGACAGCACCGACAAG
GCCGACCTGAGACTGATCTACCTGGCTCTGGCCCACATGATCAAGTTCCGGGGCCACTTTCTGATCGA
GGGCGATCTGAACCCCGACAACAGCGACGTGGACAAGCTGTTCATCCAGCTGGTGCAGACCTACAACC
AGCTGTTCGAGGAAAACCCCATCAACGCCTCTGGCGTGGACGCCAAGGCTATCCTGTCTGCCAGACTG
AGCAAGAGCAGAAGGCTGGAAAACCTGATCGCCCAGCTGCCTGGCGAGAAGAAGAATGGCCTGTTCGG
CAACCTGATTGCCCTGAGCCTGGGACTGACCCCTAACTTCAAGAGCAACTTCGACCTGGCCGAGGATG
CCAAACTGCAGCTGAGCAAGGACACCTACGACGACGACCTGGACAATCTGCTGGCCCAGATCGGCGAT
CAGTACGCCGACTTGTTTCTGGCCGCCAAGAACCTGTCCGACGCCATCCTGCTGAGCGATATCCTGAG
AGTGAACACCGAGATCACAAAGGCCCCTCTGAGCGCCTCTATGATCAAGAGATACGACGAGCACCACC
AGGATCTGACCCTGCTGAAGGCCCTCGTTAGACAGCAGCTGCCAGAGAAGTACAAAGAGATTTTCTTC
GATCAGTCCAAGAACGGCTACGCCGGCTACATTGATGGCGGAGCCAGCCAAGAGGAATTCTACAAGTT
CATCAAGCCCATCCTGGAAAAGATGGACGGCACCGAGGAACTGCTGGTCAAGCTGAACAGAGAGGACC
TGCTGCGGAAGCAGCGGACCTTCGACAATGGCTCTATCCCTCACCAGATCCACCTGGGAGAGCTGCAC
GCCATTCTGCGGAGACAAGAGGACTTTTACCCATTCCTGAAGGACAACCGGGAAAAGATCGAGAAGAT
CCTGACCTTCAGGATCCCCTACTACGTGGGACCACTGGCCAGAGGCAATAGCAGATTCGCCTGGATGA
CCAGAAAGAGCGAGGAAACCATCACACCCTGGAACTTCGAGGAAGTGGTGGACAAGGGCGCCAGCGCT
CAGTCCTTCATCGAGCGGATGACCAACTTCGATAAGAACCTGCCTAACGAGAAGGTGCTGCCCAAGCA
CTCCCTGCTGTATGAGTACTTCACCGTGTACAACGAGCTGACCAAAGTGAAATACGTGACCGAGGGAA
TGAGAAAGCCCGCCTTTCTGAGCGGCGAGCAGAAAAAGGCCATTGTGGATCTGCTGTTCAAGACCAAC
CGGAAAGTGACCGTGAAGCAGCTGAAAGAGGACTACTTCAAGAAAATCGAGTGCTTCGACAGCGTGGA
AATCAGCGGCGTGGAAGATCGGTTCAATGCCAGCCTGGGCACATACCACGACCTGCTGAAAATTATCA
AGGACAAGGACTTCCTGGACAACGAAGAGAACGAGGACATTCTCGAGGACATCGTGCTGACCCTGACA
CTGTTTGAGGACAGAGAGATGATCGAGGAACGGCTGAAAACATACGCCCACCTGTTCGACGACAAAGT
GATGAAGCAACTGAAGCGGAGGCGGTACACAGGCTGGGGCAGACTGTCTCGGAAGCTGATCAACGGCA
TCCGGGATAAGCAGTCCGGCAAGACAATCCTGGATTTCCTGAAGTCCGACGGCTTCGCCAACAGAAAC
TTCATGCAGCTGATCCACGACGACAGCCTGACCTTTAAAGAGGACATCCAGAAAGCCCAGGTGTCCGG
CCAAGGCGATTCTCTGCACGAGCACATTGCCAACCTGGCCGGATCTCCCGCCATTAAGAAGGGCATCC
TGCAGACAGTGAAGGTGGTGGACGAGCTTGTGAAAGTGATGGGCAGACACAAGCCCGAGAACATCGTG
ATCGAAATGGCCAGAGAGAACCAGACCACACAGAAGGGCCAGAAGAACAGCCGCGAGAGAATGAAGCG
GATCGAAGAGGGCATCAAAGAGCTGGGCAGCCAGATCCTGAAAGAACACCCCGTGGAAAACACCCAGC
TGCAGAACGAGAAGCTGTACCTGTACTACCTGCAGAATGGACGGGATATGTACGTGGACCAAGAGCTG
GACATCAACCGGCTGAGCGACTACGATGTGGACCATATCGTGCCCCAGAGCTTTCTGAAGGACGACTC
CATCGATAACAAGGTCCTGACCAGAAGCGACAAGAACCGGGGCAAGAGCGATAACGTGCCCTCCGAAG
AGGTGGTCAAGAAGATGAAGAACTACTGGCGACAGCTGCTGAACGCCAAGCTGATTACCCAGCGGAAG
TTCGATAACCTGACCAAGGCCGAGAGAGGCGGCCTGAGCGAACTTGATAAGGCCGGCTTCATTAAGCG
GCAGCTGGTGGAAACCCGGCAGATCACCAAACACGTGGCACAGATTCTGGACTCCCGGATGAACACTA
AGTACGACGAGAATGACAAGCTGATCCGGGAAGTGAAAGTCATCACCCTGAAGTCTAAGCTGGTGTCC
GATTTCCGGAAGGATTTCCAGTTCTACAAAGTGCGGGAAATCAACAACTACCATCACGCCCACGACGC
CTACCTGAATGCCGTTGTTGGAACAGCCCTGATCAAGAAGTATCCCAAGCTGGAAAGCGAGTTCGTGT
ACGGCGACTACAAGGTGTACGACGTGCGGAAGATGATCGCCAAGAGCGAACAAGAGATCGGCAAGGCT
ACCGCCAAGTACTTTTTCTACAGCAACATCATGAACTTTTTCAAGACAGAGATCACCCTGGCCAACGG
CGAGATCCGGAAAAGACCCCTGATCGAGACAAACGGCGAAACCGGGGAGATCGTGTGGGATAAGGGCA
GAGATTTTGCCACAGTGCGGAAAGTGCTGAGCATGCCCCAAGTGAATATCGTGAAGAAAACCGAGGTG
CAGACAGGCGGCTTCAGCAAAGAGTCTATCCTGCCTAAGCGGAACAGCGATAAGCTGATCGCCAGAAA
GAAGGACTGGGACCCTAAGAAGTACGGCGGCTTCGATAGCCCTACCGTGGCCTATTCTGTGCTGGTGG
TGGCCAAAGTGGAAAAGGGCAAGTCCAAAAAGCTCAAGAGCGTGAAAGAGCTGCTGGGGATCACCATC
ATGGAAAGAAGCAGCTTTGAGAAGAACCCGATCGACTTTCTGGAAGCCAAGGGCTACAAAGAAGTCAA
GAAGGACCTCATCATCAAGCTCCCCAAGTACAGCCTGTTCGAGCTGGAAAATGGCCGGAAGCGGATGC
TGGCCTCAGCAGGCGAACTGCAGAAAGGCAATGAACTGGCCCTGCCTAGCAAATACGTCAACTTCCTG
TACCTGGCCAGCCACTATGAGAAGCTGAAGGGCAGCCCCGAGGACAATGAGCAAAAGCAGCTGTTTGT
GGAACAGCACAAGCACTACCTGGACGAGATCATCGAGCAGATCAGCGAGTTCTCCAAGAGAGTGATCC
TGGCCGACGCTAACCTGGATAAGGTGCTGTCTGCCTATAACAAGCACCGGGACAAGCCTATCAGAGAG
CAGGCCGAGAATATCATCCACCTGTTTACCCTGACCAACCTGGGAGCCCCTGCCGCCTTCAAGTACTT
CGACACCACCATCGACCGGAAGAGGTACACCAGCACCAAAGAGGTGCTGGACGCCACACTGATCCACC
AGTCTATCACCGGCCTGTACGAAACCCGGATCGACCTGTCTCAGCTCGGCGGCGATTCTGGTGGTTCT
GGCGGAAGTGGCGGATCCACCAATCTGAGCGACATCATCGAAAAAGAGACAGGCAAGCAGCTCGTGAT
CCAAGAATCCATCCTGATGCTGCCTGAAGAGGTTGAGGAAGTGATCGGCAACAAGCCTGAGTCCGACA
TCCTGGTGCACACCGCCTACGATGAGAGCACCGATGAGAACGTCATGCTGCTGACAAGCGACGCCCCT
GAGTACAAGCCTTGGGCTCTCGTGATTCAGGACAGCAATGGGGAGAACAAGATCAAGATGCTGAGCGG
AGGTAGCGGAGGCAGTGGCGGAAGCACAAACCTGTCTGATATCATTGAAAAAGAAACCGGGAAGCAAC
TGGTCATTCAAGAGTCCATTCTCATGCTCCCGGAAGAAGTCGAGGAAGTCATTGGAAACAAACCCGAG
AGCGATATTCTGGTCCACACAGCCTATGACGAGTCTACAGACGAAAACGTGATGCTCCTGACCTCTGA
CGCTCCCGAGTATAAGCCCTGGGCACTTGTTATCCAGGACTCTAACGGGGAAAACAAAATCAAAATGT
TGTCCGGCGGCAGCAAGCGGACAGCCGATGGATCTGAGTTCGAGAGCCCCAAGAAGAAACGGAAGGTg
GAGtaa

By “base editing activity” is meant acting to chemically alter a base within a polynucleotide. In one embodiment, a first base is converted to a second base. In one embodiment, the base editing activity is cytidine deaminase activity, e.g., converting target C·G to T·A. In another embodiment, the base editing activity is adenosine or adenine deaminase activity, e.g., converting A·T to G·C. In another embodiment, the base editing activity is cytidine deaminase activity, e.g., converting target C·G to T·A and adenosine or adenine deaminase activity, e.g., converting A·T to G·C.

The term “base editor system” refers to a system for editing a nucleobase of a target nucleotide sequence. In various embodiments, the base editor (BE) system comprises (1) a polynucleotide programmable nucleotide binding domain, a deaminase domain and a cytidine deaminase domain for deaminating nucleobases in the target nucleotide sequence; and (2) one or more guide polynucleotides (e.g., guide RNA) in conjunction with the polynucleotide programmable nucleotide binding domain. In various embodiments, the base editor (BE) system comprises a nucleobase editor domains selected from an adenosine deaminase or a cytidine deaminase, and a domain having nucleic acid sequence specific binding activity. In some embodiments, the base editor system comprises (1) a base editor (BE) comprising a polynucleotide programmable DNA binding domain and a deaminase domain for deaminating one or more nucleobases in a target nucleotide sequence; and (2) one or more guide RNAs in conjunction with the polynucleotide programmable DNA binding domain. In some embodiments, the polynucleotide programmable nucleotide binding domain is a polynucleotide programmable DNA binding domain. In some embodiments, the base editor is a cytidine base editor (CBE). In some embodiments, the base editor is an adenine or adenosine base editor (ABE). In some embodiments, the base editor is an adenine or adenosine base editor (ABE) or a cytidine base editor (CBE).

The term “Cas9” or “Cas9 domain” refers to an RNA guided nuclease comprising a Cas9 protein, or a fragment thereof (e.g., a protein comprising an active, inactive, or partially active DNA cleavage domain of Cas9, and/or the gRNA binding domain of Cas9). A Cas9 nuclease is also referred to sometimes as a casnl nuclease or a CRISPR (clustered regularly interspaced short palindromic repeat) associated nuclease. An exemplary Cas9, is Streptococcus pyogenes Cas9 (spCas9), the amino acid sequence of which is provided below:

MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGFDSPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRKRYTSTKEVLDATLIHQS
ITGLYETRIDLSQLGGD
(single underline: HNH domain; double underline:
RuvC domain)

The term “conservative amino acid substitution” or “conservative mutation” refers to the replacement of one amino acid by another amino acid with a common property. A functional way to define common properties between individual amino acids is to analyze the normalized frequencies of amino acid changes between corresponding proteins of homologous organisms (Schulz, G. E. and Schirmer, R. H., Principles of Protein Structure, Springer-Verlag, New York (1979)). According to such analyses, groups of amino acids can be defined where amino acids within a group exchange preferentially with each other, and therefore resemble each other most in their impact on the overall protein structure (Schulz, G. E. and Schirmer, R. H., supra). Non-limiting examples of conservative mutations include amino acid substitutions of amino acids, for example, lysine for arginine and vice versa such that a positive charge can be maintained; glutamic acid for aspartic acid and vice versa such that a negative charge can be maintained; serine for threonine such that a free —OH can be maintained; and glutamine for asparagine such that a free —NH₂ can be maintained.

The term “coding sequence” or “protein coding sequence” as used interchangeably herein refers to a segment of a polynucleotide that codes for a protein. The region or sequence is bounded nearer the 5′ end by a start codon and nearer the 3′ end with a stop codon. Stop codons useful with the base editors described herein include the following:
Glutamine CAG→TAG Stop codon
   CAA→TAA
Arginine CGA→TGA
Tryptophan TGG→TGA
   TGG→TAG
   TGG→TAA
      Coding sequences can also be referred to as open reading frames.

By “cytidine deaminase” is meant a polypeptide or fragment thereof capable of catalyzing a deamination reaction that converts an amino group to a carbonyl group. In one embodiment, the cytidine deaminase converts cytosine to uracil or 5-methylcytosine to thymine. PmCDA1, which is derived from Petromyzon marinus (Petromyzon marinus cytosine deaminase 1, “PmCDA1”), AID (Activation-induced cytidine deaminase; AICDA), which is derived from a mammal (e.g., human, swine, bovine, horse, monkey etc.), and APOBEC are exemplary cytidine deaminases.

The term “deaminase” or “deaminase domain,” as used herein, refers to a protein or enzyme that catalyzes a deamination reaction. In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytosine deaminase, catalyzing the hydrolytic deamination of cytosine to uracil. In some embodiments, the deaminase is an adenosine deaminase, which catalyzes the hydrolytic deamination of adenine to hypoxanthine. In some embodiments, the deaminase is an adenosine deaminase, which catalyzes the hydrolytic deamination of adenosine or adenine (A) to inosine (I). In some embodiments, the deaminase or deaminase domain is an adenosine deaminase, catalyzing the hydrolytic deamination of adenosine or deoxyadenosine to inosine or deoxyinosine, respectively. In some embodiments, the adenosine deaminase catalyzes the hydrolytic deamination of adenosine in deoxyribonucleic acid (DNA). The adenosine deaminase (e.g., engineered adenosine deaminase, evolved adenosine deaminase) provided herein can be from any organism, such as a bacterium. In some embodiments, the adenosine deaminase is from a bacterium, such as E. coli, S. aureus, S. typhi, S. putrefaciens, H. influenzae, or C. crescentus. In some embodiments, the adenosine deaminase is a TadA deaminase. In some embodiments, the deaminase or deaminase domain is a variant of a naturally occurring deaminase from an organism, such as a human, chimpanzee, gorilla, monkey, cow, dog, rat, or mouse. In some embodiments, the deaminase or deaminase domain does not occur in nature. For example, in some embodiments, the deaminase or deaminase domain is at least 50%, at least 55%, at least 60%, at least 65%, at least 70%, at least 75% at least 80%, at least 85%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% identical to a naturally occurring deaminase.

“Detect” refers to identifying the presence, absence or amount of the analyte to be detected. In one embodiment, a sequence alteration in a polynucleotide or polypeptide is detected. In another embodiment, the presence of indels is detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an enzyme linked immunosorbent assay (ELISA), biotin, digoxigenin, or haptens.

By “disease” is meant any condition or disorder that damages or interferes with the normal function of a cell, tissue, or organ. Examples of diseases include HBV infection, as well as related diseases and disorders, including cirrhosis, hepatocellular carcinoma (HCC), and any other disease associated with or resulting from HBV infection.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of a base editor of the invention sufficient to introduce an alteration in an HBV genome in a cell (e.g., a cell in vitro or in vivo). In one embodiment, an effective amount is the amount of a base editor required to achieve a therapeutic effect (e.g., to reduce or control an HBV infection). Such therapeutic effect need not be sufficient to alter an HBV genome in all cells of a subject, tissue or organ, but only to alter an HBV genome in about 1%, 5%, 10%, 25%, 50%, 75% or more of the cells present in a subject, tissue or organ. In one embodiment, an effective amount is sufficient to ameliorate one or more symptoms of HBV.

In some embodiments, an effective amount of a fusion protein provided herein, e.g., of a nucleobase editor comprising a nCas9 domain and a deaminase domain (e.g., adenosine deaminase, cytidine deaminase) refers to the amount that is sufficient to induce editing of a target site specifically bound and edited by the nucleobase editors described herein. As will be appreciated by the skilled artisan, the effective amount of an agent, e.g., a fusion protein, may vary depending on various factors as, for example, on the desired biological response, e.g., on the specific genome or target site to be edited, on the cell or tissue being targeted, and/or on the agent being used.

In some embodiments, an effective amount of a fusion protein provided herein, e.g., of a fusion protein comprising a nCas9 domain and a deaminase domain may refer to the amount of the fusion protein that is sufficient to induce editing of a target site specifically bound and edited by the fusion protein. As will be appreciated by the skilled artisan, the effective amount of an agent, e.g., a fusion protein, may vary depending on various factors as, for example, on the desired biological response, e.g., on the specific genome or target site to be edited, on the cell or tissue being targeted, and/or on the agent being used.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “guide RNA” or “gRNA” is meant a polynucleotide which is specific for a target sequence and can form a complex with a polynucleotide programmable nucleotide binding domain protein (e.g., Cas9 or Cpf1). In an embodiment, the guide polynucleotide is a guide RNA (gRNA). gRNAs can exist as a complex of two or more RNAs, or as a single RNA molecule. gRNAs that exist as a single RNA molecule may be referred to as single-guide RNAs (sgRNAs), although “gRNA” is used interchangeably to refer to guide RNAs that exist as either single molecules or as a complex of two or more molecules. Typically, gRNAs that exist as single RNA species comprise two domains: (1) a domain that shares homology to a target nucleic acid (e.g., and directs binding of a Cas9 complex to the target); and (2) a domain that binds a Cas9 protein. In some embodiments, domain (2) corresponds to a sequence known as a tracrRNA, and comprises a stem-loop structure. For example, in some embodiments, domain (2) is identical or homologous to a tracrRNA as provided in Jinek et al., Science 337:816-821(2012), the entire contents of which is incorporated herein by reference. Other examples of gRNAs (e.g., those including domain 2) can be found in US20160208288, entitled “Switchable Cas9 Nucleases and Uses Thereof,” and U.S. Pat. No. 9,737,604, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” An extended gRNA will bind two or more Cas9 proteins and bind a target nucleic acid at two or more distinct regions, as described herein. The gRNA comprises a nucleotide sequence that complements a target site, which mediates binding of the nuclease/RNA complex to the target site, providing the sequence specificity of the nuclease:RNA complex.

By “HBV polymerase protein” is meant a polypeptide having at least about 95% identity to a wild-type HBV polymerase amino acid sequence or fragment thereof that functions in a hepatitis B viral infection. In one embodiment, the HBV polymerase is encoded by an HBV A, B, C, D, E, F, G, or H genotype. In one embodiment, the HBV polymerase amino acid sequence is provided at UniPro Accession No. Q8B5R0-1, which is reproduced below.

```
                10 20 30 40
MPLSYQHFRR LLLLDDEAGP LEEELPRLAD EGLNRRVAED
                50 60 70 80
LNLGNLNVSI PWTHKVGNFT GLYSSTVPVF NPHWKTPSFP
                90 100 110 120
NIHLHQDIIK KCEQFVGPLT VNEKRRLQLI MPARFYPKVT
              130 140 150 160
KYLPLDKGIK PYYPEHLVNH YFQTRHYLHT LWKAGILYKR
              170 180 190
ETTHSASFCG SPYSWEQDLQ HGAESFHQQS
```

Mutations in HBV polymerase include: E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P.

Other exemplary HBV DNA polymerases include, for example, NCBI Accession No. AAB59972.1, which has the following sequence.

MPLSYQHFRKLLLLDDEAGPLEEELPRLADEGLNRRVAEDLNLGNLNVSI
PWTHKVGNFTGLYSSTVPVFNPHWKTPSFPNIHLHQDIIKKCEQFVGPLT
VNEKRRLQLIMPARFYPKVTKYLPLDKGIKPYYPEHLVNHYFQTRHYLHT
LWKAGILYKRETTHSASFCGSPYSWEQDLQHGAESFHQQSSGILSRPPVG
SSLQSKHSKSRLGLQSQQGHLARRQQGRSWSIRAGFHPTARRPFGVEPSG
SGHTTNFASKSASCLHQSPDRKAAYPAVSTFEKHSSSGHAVEFHNLSPNS
ARSQSERPVFPCWWLQFRSSKPCSDYCLSLIVNLLEDWGPCAEHGEHHIR
IPRTPSRVTGGVFLVDKNPHNTAESRLVVDFSQFSRGNYRVSWPKFAVPN
LQSLTNLLSSNLSWLSLDVSAAFYHLPLHPAAMPHLLVGSSGLSRYVARL
SSNSRILNHQHGTMPNLHDYCSRNLYVSLLLLYQTFGRKLHLYSHPIILG
FRKIPMGVGLSPFLLAQFTSAICSVVRRAFPHCLAFSYMDDVVLGAKSVQ
HLESLFTAVTNFLLSLGIHLNPNKTKRWGYSLNFMGYVIGSYGSLPQEHI
IQKIKECFRKLPINRPIDWKVCQRIVGLLGFAAPFTQCGYPALMPLYACI
QSKQAFTFSPTYKAFLCKQYLNLYPVARQRPGLCQVFADATPTGWGLVMG
HQRVRGTFSAPLPIHTAELLAACFARSRSGANIIGTDNSVVLSRKYTSYP
WLLGCAANWILRGTSFVYVPSALNPADDPSRGRLGLSRPLLRLPFRPTTG
RTSLYADSPSVPSHLPDRVHFASPLHVAWRPP

By “HBV polymerase gene” is meant a polynucleotide encoding an HBV polymerase.

By “Hepatitis B surface antigen (HBsAg) polypeptide” is meant an antigenic protein or fragment thereof having at least about 85% identity to NCBI Accession No. AAB59969.1, which functions in an HBV viral infection. An exemplary HBsAg amino acid sequence is provided below:

MENITSGFLGPLLVLQAGFFLLTRILTIPQSLDSTNTNTSLNFLGGTTVC
LGQNSQSPTSNHSPTSCPPTCPGYRTNMCLRRFIIFLFILLLCLIFLLVL
LDYQGMLPVCPLIPGSSTTSTGPCRTCMTTAQGTSMYPSCCCTKPSDGNC
TCIPIPSSWAFGKFLWEWASARFSWLSLLVPFVQWFVGLSPTVWLSVIWM
MWYWGPSLYSILSPFLPLLPIFFCLWVYI

By “HbsAg polynucleotide” is meant a polynucleotide encoding an HBsAg protein.

By “HBV X-protein” is meant a polypeptide or fragment thereof having at least about 85% identity to NCBI Accession No. AAB59970.1, which functions in an HBV viral infection. An exemplary amino acid sequence is provided below:

| | |
|---|---|
| 1 | maarlccqld pardvlclrp vgaescgrpf sgslgtlssp |
| | spsavptdhg ahlslrglpv |
| 61 | cafssagpca lrftsarrme ttvnahrmlp kvlhkrtlgl |
| | samsttdlea yfkdclfkdw |
| 121 | eelgeeirlk vfvlggcrhk lvcapapcnf ftsa |

By “core antigen precursor” is meant a polypeptide or fragment thereof having at least about 85% identity to NCBI Accession No. AAB59971.1, which functions in an HBV viral infection.

By “HBV core protein” is meant a polypeptide having at least about 95% identity to a wild-type HBV core protein amino acid sequence or fragment thereof. In an embodiment, the HBV core protein functions in a hepatitis B viral infection. In one embodiment, the HBV core protein is encoded by an HBV A, B, C, D, E, F, G, or H genotype. In one embodiment, the HBV core protein amino acid sequence is provided at NCBI GenBank Accession No. AXG50928.1, provided below:

| | |
|---|---|
| 1 | mdidpykefg asvellsflp sdffpsirdl ldtasalyre |
| | alespehcsp hhtalrqail |
| 61 | cwgelmnlat wvgsnledpa srelvvsyvn vnmglkirql |
| | lwfhiscltf gretvleylv |
| 121 | sfgvwirtpp ayrppnapil stlpettvvr rrgrsprrrt |
| | psprrrrsqs prrrrsgsre |
| 181 | sqc |

By “HBV X protein” is meant a polynucleotide encoding an HBV X-protein.

By “HBV X protein (genotype B)” is meant a polypeptide having at least about 95% identity to a wild-type HBV genotype B X protein amino acid sequence or fragment thereof. In an embodiment, the HBV X protein functions in a hepatitis B viral infection. In one embodiment, the HBV genotype B X protein amino acid sequence is provided at NCBI GenBank Accession No. BAQ95575.1, provided below:

| | |
|---|---|
| 1 | maarlccqld pardvlclrp vgaesrgrpl pgplgalppa |
| | sppvvpsdhg ahlslrglpv |
| 61 | cafssxgpca lrftsarrme ttvnahrnlp kvlhkrtlgl |
| | samsttdlea yfkdcvfxew |
| 121 | eelgeexrlk vfvlggcrhk lvcspapcnf ftsa |

By “HBV X protein (genotype C)” is meant a polypeptide having at least about 95% identity to a wild-type HBV genotype C X protein amino acid sequence or fragment thereof. In an embodiment, the HBV X protein functions in a hepatitis B viral infection. In one embodiment, the HBV genotype C X protein amino acid sequence is provided at NCBI GenBank Accession No. BAQ95563.1, provided below:

| | |
|---|---|
| 1 | maarvccqld pardvlclrp vgaesrgrpv sgpfgplpsp |
| | sssavpadyg ahlslrglpv |
| 61 | cafssagpca lrftsarrme ttvnahqvlp kllhkrtlgl |
| | samsttdlea yfkdclfkdw |
| 121 | eelgeeirlk vfvlggcrhk lvcspapcnf ftsa |

By “HBV S protein” is meant a polypeptide having at least about 95% identity to a wild-type HBV S protein amino acid sequence or fragment thereof. In an embodiment, the HBV S protein functions in a hepatitis B viral infection. In one embodiment, the HBV S protein is encoded by an HBV A, B, C, D, E, F, G, or H genotype. In one embodiment, the HBV S protein amino acid sequence is provided at NCBI GenBank Accession No. ABV02793.1, provided below:

| | |
|---|---|
| 1 | menttsgflg pllvlqagff lltrnitipq sldswwtsln |
| | flggaptcpg qnsqsptsnh |
| 61 | sptscppicp gyrwmclrrf iiflfilllc lifllvlldy |
| | qgmlpvcpll pgtsttstgp |
| 121 | cktctipaqg tsmfpsccct kpsdgnctci pipsswafar |
| | flwewasvrf swlsllvpfv |
| 181 | qwfvglsptv wlsviwmmwy wgpslynils pflpllpiff |
| | clwvyi |

The complete genome of Hepatitis B virus subtype ayw, complete genome, which includes polynucleotides encoding HBV polymerase, HBsAg protein, HBV X protein, and the core antigen precursor, is provided at GenBank Accession No. U95551.1, which is reproduced below:

| | |
|---|---|
| 1 | aattccacaa cctttcacca aactctgcaa gatcccagag tgagaggcct gtatttccct |
| 61 | gctggtggct ccagttcagg agcagtaaac cctgttccga ctactgcctc tcccttatcg |
| 121 | tcaatcttct cgaggattgg ggaccctgcg ctgaacatgg agaacatcac atcaggattc |
| 181 | ctaggacccc ttctcgtgtt acaggcgggg tttttcttgt tgacaagaat cctcacaata |
| 241 | ccgcagagtc tagactcgtg gtggacttct ctcaattttc tagggggaac taccgtgtgt |
| 301 | cttggccaaa attcgcagtc cccaacctcc aatcactcac caacctcctg tcctccaact |
| 361 | tgtcctggtt atcgctggat gtgtctgcgg cgttttatca tcttcctctt catcctgctg |
| 421 | ctatgcctca tcttcttgtt ggttcttctg gactatcaag gtatgttgcc cgtttgtcct |
| 481 | ctaattccag gatcctcaac caccagcacg ggaccatgcc gaacctgcat gactactgct |
| 541 | caaggaacct ctatgtatcc ctcctgttgc tgtaccaaac cttcggacgg aaattgcacc |
| 601 | tgtattccca tcccatcatc ctgggctttc ggaaaattcc tatgggagtg ggcctcagcc |
| 661 | cgtttctcct ggctcagttt actagtgcca tttgttcagt ggttcgtagg gctttccccc |
| 721 | actgtttggc tttcagttat atggatgatg tggtattggg ggccaagtct gtacagcatc |
| 781 | ttgagtccct ttttaccgct gttaccaatt ttcttttgtc tttgggtata catttaaacc |
| 841 | ctaacaaaac aaagagatgg ggttactctc tgaattttat gggttatgtc attggaagtt |
| 901 | atgggtcctt gccacaagaa cacatcatac aaaaaatcaa agaatgtttt agaaaacttc |
| 961 | ctattaacag gcctattgat tggaaagtat gtcaacgaat tgtgggtctt ttgggttttg |
| 1021 | ctgccccatt tacacaatgt ggttatcctg cgttaatgcc cttgtatgca tgtattcaat |
| 1081 | ctaagcaggc tttcactttc tcgccaactt acaaggcctt tctgtgtaaa caatacctga |
| 1141 | acctttaccc cgttgcccgg caacggccag gtctgtgcca agtgtttgct gacgcaaccc |
| 1201 | ccactggctg gggcttggtc atgggccatc agcgcgtgcg tggaaccttt tcggctcctc |
| 1261 | tgccgatcca tactgcggaa ctcctagccg cttgttttgc tcgcagcagg tctggagcaa |
| 1321 | acattatcgg gactgataac tctgttgtcc tctcccgcaa atatacatcg tatccatggc |
| 1381 | tgctaggctg tgctgccaac tggatcctgc gcgggacgtc ctttgtttac gtcccgtcgg |
| 1441 | cgctgaatcc tgcggacgac ccttctcggg gtcgcttggg actctctcgt ccccttctcc |
| 1501 | gtctgccgtt ccgaccgacc acggggcgca cctctcttta cgcggactcc ccgtctgtgc |
| 1561 | cttctcatct gccggaccgt gtgcacttcg cttcacctct gcacgtcgca tggagaccac |
| 1621 | cgtgaacgcc caccgaatgt tgcccaaggt cttacataag aggactcttg gactctctgc |
| 1681 | aatgtcaacg accgaccttg aggcatactt caaagactgt ttgtttaaag actgggagga |
| 1741 | gttgggggag gagattagat taaaggtctt tgtactagga ggctgtaggc ataaattggt |
| 1801 | ctgcgcacca gcaccatgca actttttcac ctctgcctaa tcatctcttg ttcatgtcct |
| 1861 | actgttcaag cctccaagct gtgccttggg tggctttggg gcatggacat cgacccttat |
| 1921 | aaagaatttg gagctactgt ggagttactc tcgtttttgc cttctgactt ctttccttca |
| 1981 | gtacgagatc ttctagatac cgcctcagct ctgtatcggg aagccttaga gtctcctgag |
| 2041 | cattgttcac ctcaccatac tgcactcagg caagcaattc tttgctgggg ggaactaatg |
| 2101 | actctagcta cctgggtggg tgttaatttg gaagatccag catctagaga cctagtagtc |
| 2161 | agttatgtca acactaatat gggcctaaag ttcaggcaac tcttgtggtt tcacatttct |
| 2221 | tgtctcactt ttggaagaga aaccgttata gagtatttgg tgtctttcgg agtgtggatt |
| 2281 | cgcactcctc cagcttatag accaccaaat gcccctatcc tatcaacact tccggaaact |
| 2341 | actgttgtta gacgacgagg caggtcccct agaagaagaa ctccctcgcc tcgcagacga |
| 2401 | aggtctcaat cgccgcgtcg cagaagatct caatctcggg aacctcaatg ttagtattcc |
| 2461 | ttggactcat aaggtgggga actttactgg tctttattct tctactgtac ctgtctttaa |
| 2521 | tcctcattgg aaaacaccat cttttcctaa tatacattta caccaagaca ttatcaaaaa |
| 2581 | atgtgaacag tttgtaggcc cacttacagt taatgagaaa agaagattgc aattgattat |
| 2641 | gcctgctagg ttttatccaa aggttaccaa atatttacca ttggataagg gtattaaacc |
| 2701 | ttattatcca gaacatctag ttaatcatta cttccaaact agacactatt tacacactct |
| 2761 | atggaaggcg ggtatattat ataagagaga aacaacacat agcgcctcat tttgtgggtc |
| 2821 | accatattct tgggaacaag atctacagca tggggcagaa tctttccacc agcaatcctc |
| 2881 | tgggattctt tcccgaccac cagttggatc cagccttcag agcaaacaca gcaaatccag |
| 2941 | attgggactt caatcccaac aaggacacct ggccagacgc caacaaggta ggagctggag |
| 3001 | cattcgggct gggtttcacc ccaccgcacg gaggcctttt ggggtggagc cctcaggctc |
| 3061 | agggcatact acaaactttg ccagcaaatc cgcctcctgc ctccaccaat cgccagacag |
| 3121 | gaaggcagcc taccccgctg tctccacctt tgagaaacac tcatcctcag gccatgcagt |
| 3181 | gg |

By “heterodimer” is meant a fusion protein comprising two domains, such as a wild type TadA domain and a variant of TadA domain (e.g., TadA*8) or two variant TadA domains (e.g., TadA*7.10 and TadA*8 or two TadA*8 domains).

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By “increases” is meant a positive alteration of at least 10%, 25%, 50%, 75%, or 100%.

The terms “inhibitor of base repair”, “base repair inhibitor”, “IBR” or their grammatical equivalents refer to a protein that is capable in inhibiting the activity of a nucleic acid repair enzyme, for example a base excision repair enzyme. In some embodiments, the IBR is an inhibitor of inosine base excision repair. Exemplary inhibitors of base repair include inhibitors of APE1, Endo III, Endo IV, Endo V, Endo VIII, Fpg, hOGGI, hNEIL1, T7 Endol, T4PDG, UDG, hSMUG1, and hAAG. In some embodiments, the base repair inhibitor is an inhibitor of Endo V or hAAG. In some embodiments, the IBR is an inhibitor of Endo V or hAAG. In some embodiments, the IBR is a catalytically inactive EndoV or a catalytically inactive hAAG. In some embodiments, the base repair inhibitor is a catalytically inactive EndoV or a catalytically inactive hAAG. In some embodiments, the base repair inhibitor is uracil glycosylase inhibitor (UGI). UGI refers to a protein that is capable of inhibiting a uracil-DNA glycosylase base-excision repair enzyme. In some embodiments, a UGI domain comprises a wild-type UGI or a fragment of a wild-type UGI. In some embodiments, the UGI proteins provided herein include fragments of UGI and proteins homologous to a UGI or a UGI fragment. In some embodiments, the base repair inhibitor is an inhibitor of inosine base excision repair. In some embodiments, the base repair inhibitor is a “catalytically inactive inosine specific nuclease” or “dead inosine specific nuclease.” Without wishing to be bound by any particular theory, catalytically inactive inosine glycosylases (e.g., alkyl adenine glycosylase (AAG)) can bind inosine, but cannot create an abasic site or remove the inosine, thereby sterically blocking the newly formed inosine moiety from DNA damage/repair mechanisms. In some embodiments, the catalytically inactive inosine specific nuclease can be capable of binding an inosine in a nucleic acid but does not cleave the nucleic acid. Non-limiting exemplary catalytically inactive inosine specific nucleases include catalytically inactive alkyl adenosine glycosylase (AAG nuclease), for example, from a human, and catalytically inactive endonuclease V (EndoV nuclease), for example, from E. coli. In some embodiments, the catalytically inactive AAG nuclease comprises an E125Q mutation or a corresponding mutation in another AAG nuclease.

An “intein” is a fragment of a protein that is able to excise itself and join the remaining fragments (the exteins) with a peptide bond in a process known as protein splicing. Inteins are also referred to as “protein introns.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Other intein systems may also be used. For example, a synthetic intein based on the dnaE intein, the Cfa-N (e.g., split intein-N) and Cfa-C (e.g., split intein-C) intein pair, has been described (e.g., in Stevens et al., J Am Chem Soc. 2016 Feb. 24; 138(7):2162-5, incorporated herein by reference). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

Exemplary nucleotide and amino acid sequences of inteins are provided.

DnaE Intein-N DNA:
TGCCTGTCATACGAAACCGAGATACTGACAGTAGAATATGGCCTTCTGCC
AATCGGGAAGATTGTGGAGAAACGGATAGAATGCACAGTTTACTCTGTCG
ATAACAATGGTAACATTTATACTCAGCCAGTTGCCCAGTGGCACGACCGG
GGAGAGCAGGAAGTATTCGAATACTGTCTGGAGGATGGAAGTCTCATTAG
GGCCACTAAGGACCACAAATTTATGACAGTCGATGGCCAGATGCTGCCTA
TAGACGAAATCTTTGAGCGAGAGTTGGACCTCATGCGAGTTGACAACCTT
CCTAT
DnaE Intein-N Protein:
CLSYETEILTVEYGLLPIGKIVEKRIECTVYSVDNNGNIYTQPVAQWHDR
GEQEVFEYCLEDGSLIRATKDHKFMTVDGQMLPIDEIFERELDLMRVDNL
PN
DnaE Intein-C DNA:
ATGATCAAGATAGCTACAAGGAAGTATCTTGGCAAACAAAACGTTTATGA
TATTGGAGTCGAAAGAGATCACAACTTTGCTCTGAAGAACGGATTCATAG
CTTCTAAT
Intein-C:
MIKIATRKYLGKQNVYDIGVERDHNFALKNGFIASN
Cfa-N DNA:
TGCCTGTCTTATGATACCGAGATACTTACCGTTGAATATGGCTTCTTGCC
TATTGGAAAGATTGTCGAAGAGAGAATTGAATGCACAGTATATACTGTAG
ACAAGAATGGTTTCGTTTACACACAGCCCATTGCTCAATGGCACAATCGC
GGCGAACAAGAAGTATTTGAGTACTGTCTCGAGGATGGAAGCATCATACG
AGCAACTAAAGATCATAAATTCATGACCACTGACGGGCAGATGTTGCCAA
TAGATGAGATATTCGAGCGGGGCTTGGATCTCAAACAAGTGGATGGATTG
CCA
Cfa-N Protein:
CLSYDTEILTVEYGFLPIGKIVEERIECTVYTVDKNGFVYTQPIAQWHNR
GEQEVFEYCLEDGSIIRATKDHKFMTTDGQMLPIDEIFERGLDLKQVDGL
P
Cfa-C DNA:
ATGAAGAGGACTGCCGATGGATCAGAGTTTGAATCTCCCAAGAAGAAGAG
GAAAGTAAAGATAATATCTCGAAAAAGTCTTGGTACCCAAAATGTCTATG
ATATTGGAGTGGAGAAAGATCACAACTTCCTTCTCAAGAACGGTCTCGTA
GCCAGCAAC
Cfa-C Protein:
MKRTADGSEFESPKKKRKVKIISRKSLGTQNVYDIGVEKDHNFLLKNGLV
ASN

Intein-N and intein-C may be fused to the N-terminal portion of the split Cas9 and the C-terminal portion of the split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N—[N-terminal portion of the split Cas9]-[intein-N]—C. In some embodiments, an intein-C is fused to the N-terminus of the C-terminal portion of the split Cas9, i.e., to form a structure of N-[intein-C]—[C-terminal portion of the split Cas9]-C. The mechanism of intein-mediated protein splicing for joining the proteins the inteins are fused to (e.g., split Cas9) is known in the art, e.g., as described in Shah et al., Chem Sci. 2014; 5(1):446-461, incorporated herein by reference. Methods for designing and using inteins are known in the art and described, for example by WO2014004336, WO2017132580, US20150344549, and US20180127780, each of which is incorporated herein by reference in their entirety.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

The term “linker”, as used herein, can refer to a covalent linker (e.g., covalent bond), a non-covalent linker, a chemical group, or a molecule linking two molecules or moieties, e.g., two components of a protein complex or a ribonucleocomplex, or two domains of a fusion protein, such as, for example, a polynucleotide programmable DNA binding domain (e.g., dCas9) and a deaminase domain ((e.g., an adenosine deaminase, a cytidine deaminase, or an adenosine deaminase and a cytidine deaminase). A linker can join different components of, or different portions of components of, a base editor system. For example, in some embodiments, a linker can join a guide polynucleotide binding domain of a polynucleotide programmable nucleotide binding domain and a catalytic domain of a deaminase. In some embodiments, a linker can join a CRISPR polypeptide and a deaminase. In some embodiments, a linker can join a Cas9 and a deaminase. In some embodiments, a linker can join a dCas9 and a deaminase. In some embodiments, a linker can join a nCas9 and a deaminase. In some embodiments, a linker can join a guide polynucleotide and a deaminase. In some embodiments, a linker can join a deaminating component and a polynucleotide programmable nucleotide binding component of a base editor system. In some embodiments, a linker can join a RNA-binding portion of a deaminating component and a polynucleotide programmable nucleotide binding component of a base editor system. In some embodiments, a linker can join a RNA-binding portion of a deaminating component and a RNA-binding portion of a polynucleotide programmable nucleotide binding component of a base editor system. A linker can be positioned between, or flanked by, two groups, molecules, or other moieties and connected to each one via a covalent bond or non-covalent interaction, thus connecting the two. In some embodiments, the linker can be an organic molecule, group, polymer, or chemical moiety. In some embodiments, the linker can be a polynucleotide. In some embodiments, the linker can be a DNA linker. In some embodiments, the linker can be a RNA linker. In some embodiments, a linker can comprise an aptamer capable of binding to a ligand. In some embodiments, the ligand may be carbohydrate, a peptide, a protein, or a nucleic acid. In some embodiments, the linker may comprise an aptamer may be derived from a riboswitch. The riboswitch from which the aptamer is derived may be selected from a theophylline riboswitch, a thiamine pyrophosphate (TPP) riboswitch, an adenosine cobalamin (AdoCbl) riboswitch, an S-adenosyl methionine (SAM) riboswitch, an SAH riboswitch, a flavin mononucleotide (FMN) riboswitch, a tetrahydrofolate riboswitch, a lysine riboswitch, a glycine riboswitch, a purine riboswitch, a GlmS riboswitch, or a pre-queosine1 (PreQ1) riboswitch. In some embodiments, a linker may comprise an aptamer bound to a polypeptide or a protein domain, such as a polypeptide ligand. In some embodiments, the polypeptide ligand may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif. In some embodiments, the polypeptide ligand may be a portion of a base editor system component. For example, a nucleobase editing component may comprise a deaminase domain and a RNA recognition motif.

In some embodiments, the linker can be an amino acid or a plurality of amino acids (e.g., a peptide or protein). In some embodiments, the linker can be about 5-100 amino acids in length, for example, about 5, 6, 7, 8, 9, 10, 11, 12, 13, 14, 15, 16, 17, 18, 19, 20, 20-30, 30-40, 40-50, 50-60, 60-70, 70-80, 80-90, or 90-100 amino acids in length. In some embodiments, the linker can be about 100-150, 150-200, 200-250, 250-300, 300-350, 350-400, 400-450, or 450-500 amino acids in length. Longer or shorter linkers can be also contemplated.

In some embodiments, a linker joins a gRNA binding domain of an RNA-programmable nuclease, including a Cas9 nuclease domain, and the catalytic domain of a nucleic-acid editing protein (e.g., cytidine or adenosine deaminase). In some embodiments, a linker joins a dCas9 and a nucleic-acid editing protein. For example, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-200 amino acids in length, for example, 5, 6, 7, 8, 9, 10, 11, 12, 13, 14, 15, 16, 17, 18, 19, 20, 25, 35, 45, 50, 55, 60, 60, 65, 70, 70, 75, 80, 85, 90, 90, 95, 100, 101, 102, 103, 104, 105, 110, 120, 130, 140, 150, 160, 175, 180, 190, or 200 amino acids in length.

In some embodiments, the domains of a base editor are fused via a linker that comprises the amino acid sequence of SGGSSGSETPGTSESATPESSGGS, SGGSSGGSSGSETPGTSESATPESSGGSSGGS, or GGSGGSPGSPAGSPTSTEEGTSESATPESGPGTSTEPSEGSAPGSPAGSPTSTEEGTSTE PSEGSAPGTSTEPSEGSAPGTSESATPESGPGSEPATSGGSGGS. In some embodiments, domains of the nucleobase editor are fused via a linker comprising the amino acid sequence SGSETPGTSESATPES, which may also be referred to as the XTEN linker. In some embodiments, the linker is 24 amino acids in length. In some embodiments, the linker comprises the amino acid sequence SGGSSGGSSGSETPGTSESATPES. In some embodiments, the linker is 40 amino acids in length. In some embodiments, the linker comprises the amino acid sequence SGGSSGGSSGSETPGTSESATPESSGGSSGGSSGGSSGGS. In some embodiments, the linker is 64 amino acids in length. In some embodiments, the linker comprises the amino acid sequence SGGSSGGSSGSETPGTSESATPESSGGSSGGSSGGSSGGSSGSETPGTSESATPESSGGS SGGS. In some embodiments, the linker is 92 amino acids in length. In some embodiments, the linker comprises the amino acid sequence

PGSPAGSPTSTEEGTSESATPESGPGTSTEPSEGSAPGSPAGSPTSTEEG
TSTEPSEGSAPGTSTEPSEGSAPGTSESATPESGPGSEPATS.

By “marker” is meant any protein or polynucleotide having an alteration in expression level or activity that is associated with a disease or disorder.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)). In some embodiments, the presently disclosed base editors can efficiently generate an “intended mutation”, such as a point mutation, in a nucleic acid (e.g., a nucleic acid within a genome of a subject) without generating a significant number of unintended mutations, such as unintended point mutations. In some embodiments, an intended mutation is a mutation that is generated by a specific base editor (e.g., cytidine base editor or adenosine base editor) bound to a guide polynucleotide (e.g., gRNA), specifically designed to generate the intended mutation.

In general, mutations made or identified in a sequence (e.g., an amino acid sequence as described herein) are numbered in relation to a reference (or wild type) sequence, i.e., a sequence that does not contain the mutations. The skilled practitioner in the art would readily understand how to determine the position of mutations in amino acid and nucleic acid sequences relative to a reference sequence.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the wild-type protein.

In some embodiments, the presently disclosed base editors can efficiently generate an “intended mutation”, such as a point mutation, in a nucleic acid (e.g., a nucleic acid within a genome of a subject) without generating a significant number of unintended mutations, such as unintended point mutations. In some embodiments, an intended mutation is a mutation that is generated by a specific base editor (e.g., cytidine base editor or adenosine base editor) bound to a guide polynucleotide (e.g., gRNA), specifically designed to generate the intended mutation.

In general, mutations made or identified in a sequence (e.g., an amino acid sequence as described herein) are numbered in relation to a reference (or wild type) sequence, i.e., a sequence that does not contain the mutations. The skilled practitioner in the art would readily understand how to determine the position of mutations in amino acid and nucleic acid sequences relative to a reference sequence.

The term “non-conservative mutations” involve amino acid substitutions between different groups, for example, lysine for tryptophan, or phenylalanine for serine, etc. In this case, it is preferable for the non-conservative amino acid substitution to not interfere with, or inhibit the biological activity of, the functional variant. The non-conservative amino acid substitution can enhance the biological activity of the functional variant, such that the biological activity of the functional variant is increased as compared to the wild-type protein.

The term “nuclear localization sequence,” “nuclear localization signal,” or “NLS” refers to an amino acid sequence that promotes import of a protein into the cell nucleus. Nuclear localization sequences are known in the art and described, for example, in Plank et al., International PCT application, PCT/EP2000/011690, filed Nov. 23, 2000, published as WO/2001/038547 on May 31, 2001, the contents of which are incorporated herein by reference for their disclosure of exemplary nuclear localization sequences. In other embodiments, the NLS is an optimized NLS described, for example, by Koblan et al., Nature Biotech. 2018 doi:10.1038/nbt. 4172. In some embodiments, an NLS comprises the amino acid sequence KRTADGSEFESPKKKRKV, KRPAATKKAGQAKKKK, KKTELQTTNAENKTKKL, KRGINDRNFWRGENGRKTR, RKSGKIAAIVVKRPRK, PKKKRKV, or MDSLLMNRRKFLYQFKNVRWAKGRRETYLC.

The term “nucleobase,” “nitrogenous base,” or “base,” used interchangeably herein, refers to a nitrogen-containing biological compound that forms a nucleoside, which in turn is a component of a nucleotide. The ability of nucleobases to form base pairs and to stack one upon another leads directly to long-chain helical structures such as ribonucleic acid (RNA) and deoxyribonucleic acid (DNA). Five nucleobases—adenine (A), cytosine (C), guanine (G), thymine (T), and uracil (U)—are called primary or canonical. Adenine and guanine are derived from purine, and cytosine, uracil, and thymine are derived from pyrimidine. DNA and RNA can also contain other (non-primary) bases that are modified. Non-limiting exemplary modified nucleobases can include hypoxanthine, xanthine, 7-methylguanine, 5,6-dihydrouracil, 5-methylcytosine (m5C), and 5-hydromethylcytosine. Hypoxanthine and xanthine can be created through mutagen presence, both of them through deamination (replacement of the amine group with a carbonyl group). Hypoxanthine can be modified from adenine. Xanthine can be modified from guanine. Uracil can result from deamination of cytosine. A “nucleoside” consists of a nucleobase and a five carbon sugar (either ribose or deoxyribose). Examples of a nucleoside include adenosine, guanosine, uridine, cytidine, 5-methyluridine (m5U), deoxyadenosine, deoxyguanosine, thymidine, deoxyuridine, and deoxycytidine. Examples of a nucleoside with a modified nucleobase includes inosine (I), xanthosine (X), 7-methylguanosine (m7G), dihydrouridine (D), 5-methylcytidine (m5C), and pseudouridine (Ψ). A “nucleotide” consists of a nucleobase, a five carbon sugar (either ribose or deoxyribose), and at least one phosphate group.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g. nucleotides and/or nucleosides). In some embodiments, “nucleic acid” refers to an oligonucleotide chain comprising three or more individual nucleotide residues. As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g. adenosine, thymidine, guanosine, cytidine, uridine, deoxyadenosine, deoxythymidine, deoxyguanosine, and deoxycytidine); nucleoside analogs (e.g., 2-aminoadenosine, 2-thiothymidine, inosine, pyrrolo-pyrimidine, 3-methyl adenosine, 5-methylcytidine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (2′—e.g., fluororibose, ribose, 2′-deoxyribose, arabinose, and hexose); and/or modified phosphate groups (e.g., phosphorothioates and 5′-N-phosphoramidite linkages).

The term “nucleic acid programmable DNA binding protein” or “napDNAbp” may be used interchangeably with “polynucleotide programmable nucleotide binding domain” to refer to a protein that associates with a nucleic acid (e.g., DNA or RNA), such as a guide nucleic acid or guide polynucleotide (e.g., gRNA), that guides the napDNAbp to a specific nucleic acid sequence. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some embodiments, the polynucleotide programmable nucleotide binding domain is a Cas9 protein. A Cas9 protein can associate with a guide RNA that guides the Cas9 protein to a specific DNA sequence that is complementary to the guide RNA. In some embodiments, the napDNAbp is a Cas9 domain, for example a nuclease active Cas9, a Cas9 nickase (nCas9), or a nuclease inactive Cas9 (dCas9). Non-limiting examples of nucleic acid programmable DNA binding proteins include, Cas9 (e.g., dCas9 and nCas9), Cas12a/Cpf1, Cas12b/C2c1, Cas12c/C2c3, Cas12d/CasY, Cas12e/CasX, Cas12g, Cas12h, and Cas12i. Non-limiting examples of Cas enzymes include Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (also known as Csn1 or Csx12), Cas10, Cas10d, Cas12a/Cpf1, Cas12b/C2c1, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, homologues thereof, or modified or engineered versions thereof. Other nucleic acid programmable DNA binding proteins are also within the scope of this disclosure, although they may not be specifically listed in this disclosure. See, e.g., Makarova et al. “Classification and Nomenclature of CRISPR-Cas Systems: Where from Here?” CRISPR J. 2018 October; 1:325-336. doi: 10.1089/crispr.2018.0033; Yan et al., “Functionally diverse type V CRISPR-Cas systems” Science. 2019 Jan. 4; 363(6422):88-91. doi: 10.1126/science.aav7271, the entire contents of each are hereby incorporated by reference.

The terms “nucleobase editing domain” or “nucleobase editing protein,” as used herein, refers to a protein or enzyme that can catalyze a nucleobase modification in RNA or DNA, such as cytosine (or cytidine) to uracil (or uridine) or thymine (or thymidine), and adenine (or adenosine) to hypoxanthine (or inosine) deaminations, as well as non-templated nucleotide additions and insertions. In some embodiments, the nucleobase editing domain is a deaminase domain (e.g., an adenine deaminase or an adenosine deaminase; or a cytidine deaminase or a cytosine deaminase). In some embodiments, the nucleobase editing domain is more than one deaminase domain (e.g., an adenine deaminase or an adenosine deaminase and a cytidine or a cytosine deaminase). In some embodiments, the nucleobase editing domain can be a naturally occurring nucleobase editing domain. In some embodiments, the nucleobase editing domain can be an engineered or evolved nucleobase editing domain from the naturally occurring nucleobase editing domain. The nucleobase editing domain can be from any organism, such as a bacterium, human, chimpanzee, gorilla, monkey, cow, dog, rat, or mouse.

As used herein, “obtaining” as in “obtaining an agent” includes synthesizing, purchasing, or otherwise acquiring the agent.

A “patient” or “subject” as used herein refers to a mammalian subject or individual diagnosed with, at risk of having or developing, or suspected of having or developing a disease or a disorder. In some embodiments, the term “patient” refers to a mammalian subject with a higher than average likelihood of developing a disease or a disorder. Exemplary patients can be humans, non-human primates, cats, dogs, pigs, cattle, cats, horses, camels, llamas, goats, sheep, rodents (e.g., mice, rabbits, rats, or guinea pigs) and other mammalians that can benefit from the therapies disclosed herein. Exemplary human patients can be male and/or female.

“Patient in need thereof” or “subject in need thereof” is referred to herein as a patient diagnosed with, at risk or having, predetermined to have, or suspected of having a disease or disorder.

The terms “protein,” “peptide,” “polypeptide,” and their grammatical equivalents are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide can refer to an individual protein or a collection of proteins. One or more of the amino acids in a protein, peptide, or polypeptide can be modified, for example, by the addition of a chemical entity such as a carbohydrate group, a hydroxyl group, a phosphate group, a farnesyl group, an isofarnesyl group, a fatty acid group, a linker for conjugation, functionalization, or other modifications, etc. A protein, peptide, or polypeptide can also be a single molecule or can be a multi-molecular complex. A protein, peptide, or polypeptide can be just a fragment of a naturally occurring protein or peptide. A protein, peptide, or polypeptide can be naturally occurring, recombinant, or synthetic, or any combination thereof. The term “fusion protein” as used herein refers to a hybrid polypeptide which comprises protein domains from at least two different proteins. One protein can be located at the amino-terminal (N-terminal) portion of the fusion protein or at the carboxy-terminal (C-terminal) protein thus forming an amino-terminal fusion protein or a carboxy-terminal fusion protein, respectively. A protein can comprise different domains, for example, a nucleic acid binding domain (e.g., the gRNA binding domain of Cas9 that directs the binding of the protein to a target site) and a nucleic acid cleavage domain, or a catalytic domain of a nucleic acid editing protein. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA or DNA. Any of the proteins provided herein can be produced by any method known in the art. For example, the proteins provided herein can be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference.

Polypeptides and proteins disclosed herein (including functional portions and functional variants thereof) can comprise synthetic amino acids in place of one or more naturally-occurring amino acids. Such synthetic amino acids are known in the art, and include, for example, aminocyclohexane carboxylic acid, norleucine, α-amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, α-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N′,N′-dibenzyl-lysine, 6-hydroxylysine, ornithine, α-aminocyclopentane carboxylic acid, α-aminocyclohexane carboxylic acid, α-aminocycloheptane carboxylic acid, α-(2-amino-2-norbornane)-carboxylic acid, α,γ-diaminobutyric acid, α,β-diaminopropionic acid, homophenylalanine, and α-tert-butylglycine. The polypeptides and proteins can be associated with post-translational modifications of one or more amino acids of the polypeptide constructs. Non-limiting examples of post-translational modifications include phosphorylation, acylation including acetylation and formylation, glycosylation (including N-linked and O-linked), amidation, hydroxylation, alkylation including methylation and ethylation, ubiquitylation, addition of pyrrolidone carboxylic acid, formation of disulfide bridges, sulfation, myristoylation, palmitoylation, isoprenylation, farnesylation, geranylation, glypiation, lipoylation and iodination.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

By “reduces” is meant a negative alteration of at least 10%, 25%, 50%, 75%, or 100%.

By “reference” is meant a standard or control condition. In one embodiment, the viral load present in a cell treated with a base editor system described herein is compared to the level of HBV infection present in an untreated control cell, which control serves as a reference. In another embodiment, the sequence of an HBV genome present in cell contacted with a base editor system described herein is compared to the sequence of an HBV genome present in an untreated control cell.

A “reference sequence” is a defined sequence used as a basis for sequence comparison. A reference sequence may be a subset of or the entirety of a specified sequence; for example, a segment of a full-length cDNA or gene sequence, or the complete cDNA or gene sequence. For polypeptides, the length of the reference polypeptide sequence will generally be at least about 16 amino acids, at least about 20 amino acids, at least about 25 amino acids, about 35 amino acids, about 50 amino acids, or about 100 amino acids. For nucleic acids, the length of the reference nucleic acid sequence will generally be at least about 50 nucleotides, at least about 60 nucleotides, at least about 75 nucleotides, about 100 nucleotides, or about 300 nucleotides or any integer thereabout or therebetween. In some embodiments, a reference sequence is a wild-type sequence of a protein of interest. In other embodiments, a reference sequence is a polynucleotide sequence encoding a wild-type protein.

The term “RNA-programmable nuclease,” and “RNA-guided nuclease” are used with (e.g., binds or associates with) one or more RNA(s) that is not a target for cleavage. In some embodiments, an RNA-programmable nuclease, when in a complex with an RNA, may be referred to as a nuclease:RNA complex. Typically, the bound RNA(s) is referred to as a guide RNA (gRNA). In some embodiments, the RNA-programmable nuclease is the (CRISPR-associated system) Cas9 endonuclease, for example, Cas9 (Csn1) from Streptococcus pyogenes (See, e.g., “Complete genome sequence of an M1 strain of Streptococcus pyogenes.” Ferretti J. J., McShan W. M., Ajdic D. J., Savic D. J., Savic G., Lyon K., Primeaux C, Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C M., Gonzales K., Chao Y., Pirzada Z. A., Eckert M. R., Vogel J., Charpentier E., Nature 471:602-607(2011).

By “specifically binds” is meant a nucleic acid molecule, polypeptide, or complex thereof (e.g., a nucleic acid programmable DNA binding domain and guide nucleic acid), compound, or molecule that recognizes and binds a polypeptide and/or nucleic acid molecule of the invention, but which does not substantially recognize and bind other molecules in a sample, for example, a biological sample.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will be less than about 30 mM NaCl and 3 mM trisodium citrate or less than about 15 mM NaCl and 1.5 mM trisodium citrate. Stringent temperature conditions for the wash steps will ordinarily include a temperature of at least about 25° C., at least about 42° C. or even at least about 68° C. In an embodiment, wash steps will occur at 25° C. in 30 mM NaCl, 3 mM trisodium citrate, and 0.1% SDS. In another embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “split” is meant divided into two or more fragments.

A “split Cas9 protein” or “split Cas9” refers to a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a “reconstituted” Cas9 protein. In particular embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871. PDB file: 5F9R, each of which is incorporated herein by reference. In some embodiments, the protein is divided into two fragments at any C, T, A, or S within a region of SpCas9 between about amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as “splitting” the protein.

In other embodiments, the N-terminal portion of the Cas9 protein comprises amino acids 1-573 or 1-637 S. pyogenes Cas9 wild-type (SpCas9) (NCBI Reference Sequence: NC_002737.2, Uniprot Reference Sequence: Q99ZW2) and the C-terminal portion of the Cas9 protein comprises a portion of amino acids 574-1368 or 638-1368 of SpCas9 wild-type.

The C-terminal portion of the split Cas9 can be joined with the N-terminal portion of the split Cas9 to form a complete Cas9 protein. In some embodiments, the C-terminal portion of the Cas9 protein starts from where the N-terminal portion of the Cas9 protein ends. As such, in some embodiments, the C-terminal portion of the split Cas9 comprises a portion of amino acids (551-651)-1368 of spCas9. “(551-651)-1368” means starting at an amino acid between amino acids 551-651 (inclusive) and ending at amino acid 1368. For example, the C-terminal portion of the split Cas9 may comprise a portion of any one of amino acid 551-1368, 552-1368, 553-1368, 554-1368, 555-1368, 556-1368, 557-1368, 558-1368, 559-1368, 560-1368, 561-1368, 562-1368, 563-1368, 564-1368, 565-1368, 566-1368, 567-1368, 568-1368, 569-1368, 570-1368, 571-1368, 572-1368, 573-1368, 574-1368, 575-1368, 576-1368, 577-1368, 578-1368, 579-1368, 580-1368, 581-1368, 582-1368, 583-1368, 584-1368, 585-1368, 586-1368, 587-1368, 588-1368, 589-1368, 590-1368, 591-1368, 592-1368, 593-1368, 594-1368, 595-1368, 596-1368, 597-1368, 598-1368, 599-1368, 600-1368, 601-1368, 602-1368, 603-1368, 604-1368, 605-1368, 606-1368, 607-1368, 608-1368, 609-1368, 610-1368, 611-1368, 612-1368, 613-1368, 614-1368, 615-1368, 616-1368, 617-1368, 618-1368, 619-1368, 620-1368, 621-1368, 622-1368, 623-1368, 624-1368, 625-1368, 626-1368, 627-1368, 628-1368, 629-1368, 630-1368, 631-1368, 632-1368, 633-1368, 634-1368, 635-1368, 636-1368, 637-1368, 638-1368, 639-1368, 640-1368, 641-1368, 642-1368, 643-1368, 644-1368, 645-1368, 646-1368, 647-1368, 648-1368, 649-1368, 650-1368, or 651-1368 of spCas9. In some embodiments, the C-terminal portion of the split Cas9 protein comprises a portion of amino acids 574-1368 or 638-1368 of SpCas9.

By “subject” is meant a mammal, including, but not limited to, a human or non-human mammal, such as a non-human primate (monkey), bovine, equine, canine, ovine, or feline. In some embodiments, a subject described herein is infected with HBV or has a propensity to develop HBV.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). In one embodiment, such a sequence is at least 60%, 80%, 85%, 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. COBALT is used, for example, with the following parameters:
a) alignment parameters: Gap penalties-11,-1 and End-Gap penalties-5,-1,
b) CDD Parameters: Use RPS BLAST on; Blast E-value 0.003; Find Conserved columns and Recompute on, and
c) Query Clustering Parameters: Use query clusters on; Word Size 4; Max cluster distance 0.8; Alphabet Regular.
   EMBOSS Needle is used, for example, with the following parameters:
a) Matrix: BLOSUM62;
b) GAP OPEN: 10;
c) GAP EXTEND: 0.5;
d) OUTPUT FORMAT: pair;
e) END GAP PENALTY: false;
f) END GAP OPEN: 10; and
g) END GAP EXTEND: 0.5.

The term “target site” refers to a sequence within a nucleic acid molecule that is deaminated by a deaminase or a fusion protein comprising a deaminase (e.g., cytidine or adenine deaminase) fusion protein or a base editor disclosed herein).

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et ah, Multiplex genome engineering using CRISPR/Cas systems. Science 339, 819-823 (2013); Mali, P. et ah, RNA-guided human genome engineering via Cas9. Science 339, 823-826 (2013); Hwang, W. Y. et ah, Efficient genome editing in zebrafish using a CRISPR-Cas system. Nature biotechnology 31, 227-229 (2013); Jinek, M. et ah, RNA-programmed genome editing in human cells. eLife 2, e00471 (2013); Dicarlo, J. E. et ah, Genome engineering in Saccharomyces cerevisiae using CRISPR-Cas systems. Nucleic acids research (2013); Jiang, W. et ah RNA-guided editing of bacterial genomes using CRISPR-Cas systems. Nature biotechnology 31, 233-239 (2013); the entire contents of each of which are incorporated herein by reference).

As used herein, the terms “treat,” treating,” “treatment,” and the like refer to reducing or ameliorating a disorder and/or symptoms associated therewith or obtaining a desired pharmacologic and/or physiologic effect. It will be appreciated that, although not precluded, treating a disorder or condition does not require that the disorder, condition or symptoms associated therewith be completely eliminated. In some embodiments, the effect is therapeutic, i.e., without limitation, the effect partially or completely reduces, diminishes, abrogates, abates, alleviates, decreases the intensity of, or cures a disease and/or adverse symptom attributable to the disease. In some embodiments, the effect is preventative, i.e., the effect protects or prevents an occurrence or reoccurrence of a disease or condition. To this end, the presently disclosed methods comprise administering a therapeutically effective amount of a compositions as described herein. In one embodiment, the invention provides for the treatment of HBV infection.

By “uracil glycosylase inhibitor” or “UGI” is meant an agent that inhibits the uracil-excision repair system. In one embodiment, the agent is a protein or fragment thereof that binds a host uracil-DNA glycosylase and prevents removal of uracil residues from DNA. In an embodiment, a UGI is a protein, a fragment thereof, or a domain that is capable of inhibiting a uracil-DNA glycosylase base-excision repair enzyme. In some embodiments, a UGI domain comprises a wild-type UGI or a modified version thereof. In some embodiments, a UGI domain comprises a fragment of the exemplary amino acid sequence set forth below. In some embodiments, a UGI fragment comprises an amino acid sequence that comprises at least 60%, at least 65%, at least 70%, at least 75%, at least 80%, at least 85%, at least 90%, at least 95%, at least 96%, at least 97%, at least 98%, at least 99%, or 100% of the exemplary UGI sequence provided below. In some embodiments, a UGI comprises an amino acid sequence that is homologous to the exemplary UGI amino acid sequence or fragment thereof, as set forth below. In some embodiments, the UGI, or a portion thereof, is at least 70%, at least 75%, at least 80%, at least 85%, at least 90%, at least 95%, at least 96%, at least 97%, at least 98%, at least 99%, at least 99.5%, at least 99.9%, or 100% identical to a wild type UGI or a UGI sequence, or portion thereof, as set forth below. An exemplary UGI comprises an amino acid sequence as follows:

>sp1P14739IUNGI_BPPB2 Uracil-DNA glycosylase
inhibitor
MTNLSDIIEKETGKQLVIQESILMLPEEVEEVIGNKPESDILVHTAYDES
TDENVMLLT SD APE YKPW ALVIQDS NGENKIKML.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

The description and examples herein illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope.

All terms are intended to be understood as they would be understood by a person skilled in the art. Unless defined otherwise, all technical and scientific terms used herein have the same meaning as commonly understood by one of ordinary skill in the art to which the disclosure pertains.

The practice of some embodiments disclosed herein employ, unless otherwise indicated, conventional techniques of immunology, biochemistry, chemistry, molecular biology, microbiology, cell biology, genomics and recombinant DNA, which are within the skill of the art. See for example Sambrook and Green, Molecular Cloning: A Laboratory Manual, 4th Edition (2012); the series Current Protocols in Molecular Biology (F. M. Ausubel, et al. eds.); the series Methods In Enzymology (Academic Press, Inc.), PCR 2: A Practical Approach (M. J. MacPherson, B. D. Hames and G. R. Taylor eds. (1995)), Harlow and Lane, eds. (1988) Antibodies, A Laboratory Manual, and Culture of Animal Cells: A Manual of Basic Technique and Specialized Applications, 6th Edition (R. I. Freshney, ed. (2010)).

Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment. The section headings used herein are for organizational purposes only and are not to be construed as limiting the subject matter described.

The features of the present disclosure are set forth with particularity in the appended claims. A better understanding of the features and advantages of the present will be obtained by reference to the following detailed description that sets forth illustrative embodiments, in which the principles of the disclosure are utilized, and in view of the accompanying drawings as described hereinbelow.

### BRIEF DESCRIPTION OF THE DRAWINGS

FIG. 1 is an illustration showing the partially double-stranded and the overlapping open reading frames (ORFs) for the hepatitis B surface antigen (HBsAg) gene, the polymerase gene, the protein X gene, and the core gene. The HBsAg gene comprises ORF PreS1, ORF PreS2, and ORF S, which encode the large, middle, and small surface proteins, respectively. ORF core and Pre C encode capsid proteins.

FIG. 2 is an illustration depicting the HBV life cycle. The term “ER” denotes endoplasmic reticulum. The term “HBsAg” denotes hepatitis B surface antigen. “HBx transcriptional activator” is an HBV-specific transcriptional activator of polymerase II and III promoters.

FIG. 3A is a map of the geographic distribution of hepatitis B virus genotypes worldwide.

FIG. 3B provides a summary of a base editing strategies for introducing stop codons in viral genes and for generating abasic sites to treat chronic HBV.

FIG. 3C provides a summary of guide RNA screening strategies adapted for introducing stop codons and for generating abasic via base editing.

FIG. 3D is a diagram illustrating conserved gRNA design for generating abasic sites in cccDNA.

FIG. 3E is a diagram of the HBV cccDNA showing the relative position of 16 guide RNAs (depicted as triangles) that are expected to generate an amino acid that occurs in less than 0.05% of HBV genomes.

FIG. 3F is a graph showing the highest percentage of base editing generated by gRNA candidates.

FIG. 3G is a chart summarizing information relating to gRNA candidates.

FIGS. 4A and 4B depict base editors. FIG. 4A is a depiction of a base editor having an APOBEC cytidine deaminase domain, a Cas9 domain, and two uracil glycosylase inhibitor (UGI) domains. FIG. 4B provides a diagram of BE4.

FIG. 5 is an illustration showing where guide RNAs of the present disclosure map to the HBV genome. Each triangle represents a unique guide RNA.

FIG. 6 is a schematic illustration summarizing the screen for guide RNA molecules that target an HBV gene and a subset of observed results from the screen. “PAM” refers to protospacer adjacent motif “Pol” refers to the HBV polymerase gene; “S” refers to the HBV surface protein; “X” refers to the HBV protein X gene; and “Core” refers to the HBV core protein. MSPbeam52, 50, . . . , etc. refer to guide RNA, which are also termed M52, M50, . . . etc., in the application. The screen identified 12 gRNAs that exhibited greater than 20% on-target base editing.

FIG. 7 comprises graphs comparing the BE4 and A3ABE4 base editors. The graphs show the percent editing observed for different guide RNAs used with each base editor. MSPbeam39, 40, . . . , etc. are also termed M39, M40, . . . , etc., in the application.

FIG. 8 is a graph illustrating functional base editing observed in a HepG2-NTCP cell line transduced with an HBV lentiviral vector and transfected with a nucleic acid construct encoding a base editor. The nucleic acid constructs tested were DNA molecules, wild type RNA molecules, or RNA molecules comprising pseudo-uridine (PsU) modified at the N1 residue. “NTCP” refers to sodium taurocholate co-transporting polypeptide. MSPbeam39, 40, . . . , etc. are also termed M39, M40, . . . , etc., in the application.

FIG. 9 is a graph illustrating functional base editing observed in a HepG2-NTCP cell line transduced with an HBV lentiviral vector and transfected with a nucleic acid construct encoding a base editor. The nucleic acid constructs tested were DNA format transfection (two plasmids one encoding the base editor and one encoding the gRNA) or RNA format (PsU-modified in-house mRNA encoding the base editor where the RNA is modified at the N1 residue and a synthetic gRNA). Up to 80% editing in HepG2-NTCP lenti HBV cell lines was observed when using base editors and lead Stop/Functional Change (“FC”) gRNAs in an RNA format. “NTCP” refers to sodium taurocholate co-transporting polypeptide. MSPbeam39, 40, . . . , etc. are also termed M39, M40, . . . , etc., in the application.

FIG. 10 is a graph illustrating functional base editing observed in a HepG2-NTCP cell line transduced with an HBV lentiviral vector and transfected with one of three nucleic acid constructs encoding a BE4, BE4-VRQR, or ABE base editor. MSPbeam39, 40, . . . , etc. are also termed M39, M40, . . . , etc., in the application.

FIG. 11 is an illustration depicting guide RNAs that map to conserved regions of the HBV genome.

FIG. 12A is a schematic illustrating long-term primary hepatocyte co-cultures. FIG. 12B provides an experimental timeline for hepatocyte monolayers or hepatocyte co-cultures.

FIG. 12C shows images of transduced primary hepatocytes from donors (RSE, TVR) used in the co-culture system.

FIGS. 13A-13F characterize an HBV-infected primary human hepatocyte (PHH) system. FIG. 13A is a timeline showing the infection and treatment schedule for the 13 days from plating to study end-point. FIG. 13B is a graph showing the amount of extracellular HBV DNA present in a PHH culture after no treatment of HBV infected PHH cells, treatment with interferon, or treatment with tenofovir. As a negative control, PHH cells were exposed to the HBV virus without polyethylene glycol. FIG. 13C is a graph showing the amount of HBV surface antigen (HBsAg) present in PHH cultures exposed to the experimental conditions described in the legend for FIGS. 13B and 13C. FIG. 13D is a graph showing the amount of intracellular HBV DNA present in PHH cultures exposed to the experimental conditions described in the legend for FIGS. 13B and 13C. FIG. 13E is a graph showing the amount of total HBV RNA present in PHH cultures exposed to the experimental conditions described in the legend for FIGS. 13B and 13C. FIG. 13F is a graph showing the amount of pregenomic RNA (pgRNA) present in PHH cultures exposed to the experimental conditions described in the legend for FIGS. 13B and 13C.

FIG. 14 is a graph showing that transfection with a BE4 and gRNAs leads to a decrease in HBV marker levels in HBV infected PHH. Guide RNAs 52 and 190, which target the BE4 base editor to the Pol and X gene regions of the HBV genome, respectively, were used. BE4 was tested with and without a Uridine Glycosylase Inhibitor (UGI) domain.

FIG. 15 is a graph showing the identification of functional guide RNAs in a screen in HBV-infected PHH cells, where decreased levels of HBsAg, which is a surrogate of cccDNA, are indicative of a functional gRNA. Guide RNAs introducing stop codons are noted as Stop-39, etc. . . . , Guide RNAs introducing changes at conserved amino acids are indicated as Conserved-4, etc. . . . gRNAs (Stop-191, Conserved-12) selected for further analysis are indicated with boxes.

FIGS. 16A and 16B illustrate mechanistic aspects of base editing action on HBV. FIG. 16A is a graph showing the levels of HBsAg in HBV infected PHH cells transfected with mRNA encoding either a BE4 base editor with a UGI domain (BE4), a BE4 base editor with no UGI domain (BE4_noUGI), Cas9, a catalytically dead (i.e., having no nickase activity) BE4 base editor with no UGI domain (dBE4_noUGI), or a dead Cas9 (dCas9). The cells were transfected with mRNA encoding the base editor only, or were also transfected with either gRNA191 or gRNA12. FIG. 16B is a graph showing the levels of extracellular HBV DNA in HBV infected PHH cells transfected as described for FIG. 16A.

FIGS. 17A and 17B compare base editing in HepG2-NTCP Lenti-HBV and HBV infected PHH. FIG. 17A is a graph showing the editing efficiencies observed in HepG2-NTCP Lenti-HBV transfected with BE4 and UGI versus BE4 without UGI. FIG. 17B is a graph showing the editing efficiencies observed in HBV infected PHH transfected with BE4 and UGI versus BE4 without UGI.

FIG. 18 is a graph comparing the base editing, indel rates, and transversion rates (i.e., C to A or G) using gRNA190 in HBV-Lenti-HepG2 versus HBV infected PHH.

FIG. 19 shows a schematic timeline related to the use of primary hepatocyte co-culture (PHH) infected with HBV virus as a clinically relevant system for assessing anti-viral activity of the base editing reagents described herein. In some embodiments, PHH co-cultures infected with HBV were used in the experiments described herein to assay and assess the antiviral efficacy of the base editors. In brief, the base editing reagents (base editor mRNA and synthetic gRNA) were transfected into PHH co-cultures via lipofection twice over the course of two weeks. The first transfection was performed 3 days after infection with HBV to ensure that the cccDNA was completely formed at the time of virus transfection. Extracellular parameters (HBsAg, HBeAg, and HBV DNA) were monitored over the course of the described experiments, and intracellular parameters (HBV DNA, viral RNA, and editing) were monitored at the end of the described experiments. HbsAg refers to the surface protein antigen of HBV. Its detection indicates HBV infection in an individual. HBeAg refers to the hepatitis B e-antigen, a HBV protein antigen that circulates in infected blood when the virus is actively replicating. The presence of FHBeAg suggests that an individual is infectious and is able to spread the virus to others.

FIG. 20 shows a bar graph presenting the results of a 14-day experiment employing HBV-infected primary hepatocyte co-cultures (PHH) and gRNA12, which targets a polynucleotide sequence in the intersection of the HBV Polymerase and S gene sequences. The antiviral drug entecavir was used as a control to assess the efficacy of the base editors (BE4 and BE4-noUGI). As observed, the BE4-noUGI base editor and the gRNA12 resulted in a reduction of all 4 viral marker parameters tested, namely, a reduction in the amounts or levels of the HBV DNA, HBsAg, HBeAg and pgRNA marker parameters. In addition, the BE4-noUGI base editor and the gRNA12 showed an overall superior performance in reducing all 4 HBV parameters tested compared with entecavir. Accordingly, the base editing approach described herein was demonstrated to be more efficient in reducing the viral (HBV) parameters tested compared with the HBV antiviral drug entecavir.

FIG. 21 shows a bar graph presenting the results of employing multiple gRNAs (gRNA multiplexing) in conjunction with BE4. The HBV parameters assessed included pgRNA, HBsAg, HBeAg and HBV total DNA. The results indicate a gRNA-specific reduction in particular HBV parameters, with gRNA19 demonstrating an improved HBV inhibition activity compared with other gRNAs tested. In addition, a measurable improvement in HBV inhibition was observed using gRNA multiplexing, particularly with the combination of gRNA19+gRNA190, and with a combination of gRNA190, gRNA12, gRNA40 and gRNA52, which showed optimal HBV inhibition activities.

FIG. 22 shows a bar graph presenting the results of base editing using the HBV-infected PHH culture system and the BE4 base editor. NGS sequencing was performed on the total DNA purified from HBV-infected PHH cultures and on the same samples enriched for cccDNA. The results demonstrated significantly increased base editing (% base editing) in cccDNA enriched samples, thus indicating the successful base editing of HBV cccDNA by the BE4 base editor and gRNAs. The finding of reduced base editing in total genomic DNA purified from HBV-PHH suggests the inability of edited cccDNA to propagate into a replication-competent viral particle.

FIG. 23 shows a bar graph presenting the results of employing multiple gRNAs (gRNA multiplexing) with BE4 and noUGI (BE4_noUGI), e.g., as described in Example 10, infra. The HBV parameters assessed included HBsAg, HBeAg, pgRNA and HBV total DNA. The results indicate a significant gRNA-specific inhibition of HBV parameters, with gRNA12 and gRNA19 demonstrating increased inhibition activities. In addition, the HBV-inhibition activity of gRNA19 with BE_noUGI was found to be equally effective as combinations of other gRNAs tested.

FIG. 24 shows a bar graph presenting the results of base editing using the HBV-infected PHH culture system and the BE4_noUGI base editor. NGS sequencing was performed on the total DNA purified from HBV-infected PHH cultures and on the same samples enriched for cccDNA. The results demonstrated significantly increased base editing (% base editing) in cccDNA enriched samples, thus indicating the successful base editing of HBV cccDNA by BE_noUGI and gRNAs. The finding of robust base editing activity in total genomic DNA purified from HBV-PHH suggests the inability of edited cccDNA to propagate into a replication-competent viral particle.

FIGS. 25A-25D show graphs and bar graphs related to the use of the base editor dBE4_noUGI (H840A) without nickase activity and the HBV-infected PHH system in a long term (e.g., 25-day) experiment to assess the efficacy of the base editor on HBV viral parameters HBsAg (FIG. 25A), extracellular HBV DNA (FIG. 25B), HBeAg (FIG. 25C), and albumin (cell viability/metabolic rate), (FIG. 25D). The results of this experiment showed that dBE4_noUGI (D10A_H840A) and gRNA12 reduced viral parameters in HBV-infected PHH. In addition, while both interferon and the base editing components (dBE4_noUGI+gRNA12) decreased HBV viral parameters, interferon treatment was found to be more toxic compared to the use of the base editor and base editing system described herein. Base editor dBE4_noUGI (H840A) comprises the amino acid sequence

MSSETGPVAVDPTLRRRIEPHEFEVFFDPRELRKETCLLYEINWGGRHSI
WRHTSQNTNKHVEVNFIEKFTTERYFCPNTRCSITWFLSWSPCGECSRAI
TEFLSRYPHVTLFIYIARLYHHADPRNRQGLRDLISSGVTIQIMTEQESG
YCWRNFVNYSPSNEAHWPRYPHLWVRLYVLELYCIILGLPPCLNILRRKQ
PQLTFFTIALQSCHYQRLPPHILWATGLKSGGSSGGSSGSETPGTSESAT
PESSGGSSGGSDKKYSIGLAIGTNSVGWAVITDEYKVPSKKFKVLGNTDR
HSIKKNLIGALLFDSGETAEATRLKRTARRRYTRRKNRICYLQEIFSNEM
AKVDDSFFHRLEESFLVEEDKKHERHPIFGNIVDEVAYHEKYPTIYHLRK
KLVDSTDKADLRLIYLALAHMIKFRGHFLIEGDLNPDNSDVDKLFIQLVQ
TYNQLFEENPINASGVDAKAILSARLSKSRRLENLIAQLPGEKKNGLFGN
LIALSLGLTPNFKSNFDLAEDAKLQLSKDTYDDDLDNLLAQIGDQYADLF
LAAKNLSDAILLSDILRVNTEITKAPLSASMIKRYDEHHQDLTLLKALVR
QQLPEKYKEIFFDQSKNGYAGYIDGGASQEEFYKFIKPILEKMDGTEELL
VKLNREDLLRKQRTFDNGSIPHQIHLGELHAILRRQEDFYPFLKDNREKI
EKILTFRIPYYVGPLARGNSRFAWMTRKSEETITPWNFEEVVDKGASAQS
FIERMTNFDKNLPNEKVLPKHSLLYEYFTVYNELTKVKYVTEGMRKPAFL
SGEQKKAIVDLLFKTNRKVTVKQLKEDYFKKIECFDSVEISGVEDRFNAS
LGTYHDLLKIIKDKDFLDNEENEDILEDIVLTLTLFEDREMIEERLKTYA
HLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQT
VKVVDELVKVMGRHKPENIVIEMARENQTTQKGQKNSRERMKRIEEGIKE
LGSQILKEHPVENTQLQNEKLYLYYLQNGRDMYVDQELDINRLSDYDVDA
IVPQSFLKDDSIDNKVLTRSDKNRGKSDNVPSEEVVKKMKNYWRQLLNAK
LITQRKFDNLTKAERGGLSELDKAGFIKRQLVETRQITKHVAQILDSRMN
TKYDENDKLIREVKVITLKSKLVSDFRKDFQFYKVREINNYHHAHDAYLN
AVVGTALIKKYPKLESEFVYGDYKVYDVRKMIAKSEQEIGKATAKYFFYS
NIMNFFKTEITLANGEIRKRPLIETNGETGEIVWDKGRDFATVRKVLSMP
QVNIVKKTEVQTGGFSKESILPKRNSDKLIARKKDWDPKKYGGFDSPTVA
YSVLVVAKVEKGKSKKLKSVKELLGITIMERSSFEKNPIDFLEAKGYKEV
KKDLIIKLPKYSLFELENGRKRMLASAGELQKGNELALPSKYVNFLYLAS
HYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTK
EVLDATLIHQSITGLYETRIDLSQLGGDSGGSKRTADGSEFESPKKKRKV
E

FIGS. 26A-26C present graphs and bar graphs showing the results of long-term (e.g., 25 day) experiments involving PHH cultures infected with HBV of genotypes D and C to assess the base editor (e.g., dBE4_no UGI) and BE system (e.g., dBE4_no UGI+gRNA, e.g., gRNA12) as described herein in reducing or inhibiting HBV by assessing HBV parameters, namely, HBsAg (FIG. 26A), HBeAg (FIG. 26B) and extracellular HBV DNA (FIG. 26C). The experiments demonstrated that HBV of genotype C infected cells more aggressively, as the viral load was higher at the termination of the experiment. In addition, transfection of HBV-infected PHH cultures with dBE4_no UGI and gRNA12 led to a reduction of viral parameters compared to controls for both HBV of genotype D and HBV of genotype C.

FIGS. 27A and 27B present bar graphs demonstrating the results of transfection of HBV-infected PHH cultures with the adenine base editor ABE7.10 and an HBV-specific gRNA, e.g., gRNA94, which targets HBV polymerase active site. As demonstrated, ABE7.10+gRNA94 showed significant gRNA-specific HBV inhibition and reduction of the HBV markers HBsAg, HBeAg, pgRNA and HBV total DNA in the assayed PHH cultures relative to controls (no treatment of PHH and ABE7.10-only treatment of PHH). (FIG. 27A). In addition, ABE7.10+gRNA94 in HBV-infected PHH resulted in robust HBV cccDNA editing. (FIG. 27B). The lack of base editing observed in total HBV genomic DNA suggests an inability of edited HBV cccDNA to propagate into a replication-competent viral particle.

### DETAILED DESCRIPTION OF THE INVENTION

The invention features compositions and methods for editing the HBV genome. For example, the compositions contemplated herein can, in some embodiments, include a base editor a guide nucleic acid that targets a particular nucleotide in an HBV gene. In some embodiments, the editing introduces a premature stop codon in the coding sequence of one of the viral proteins. In another embodiment, the editing introduces one or more functional substitutions in the coding sequence of one or more HBV proteins.

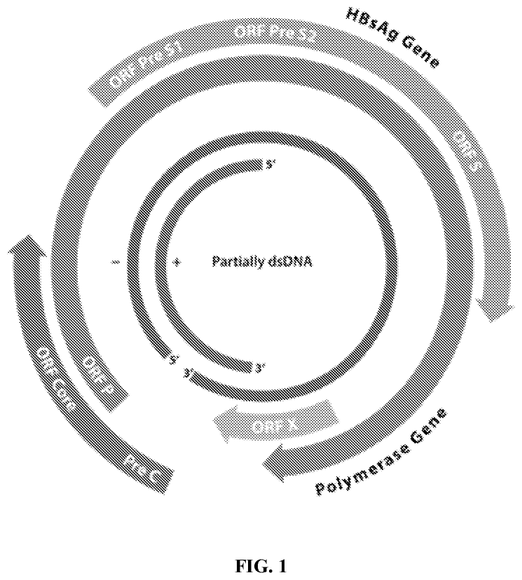

The HBV genome comprises 3.2 kb of partially double-stranded DNA and open read frames (ORFs) encoding seven proteins. Referring to FIG. 1, the open reading frame (ORF) P encodes the viral polymerase. The ORF C/PreC encodes capsid proteins. ORF PreS1, ORF PreS2, and ORF S encode large (L), middle (M) and small (S) surface proteins, respectively. ORF X encodes the secretary X protein.

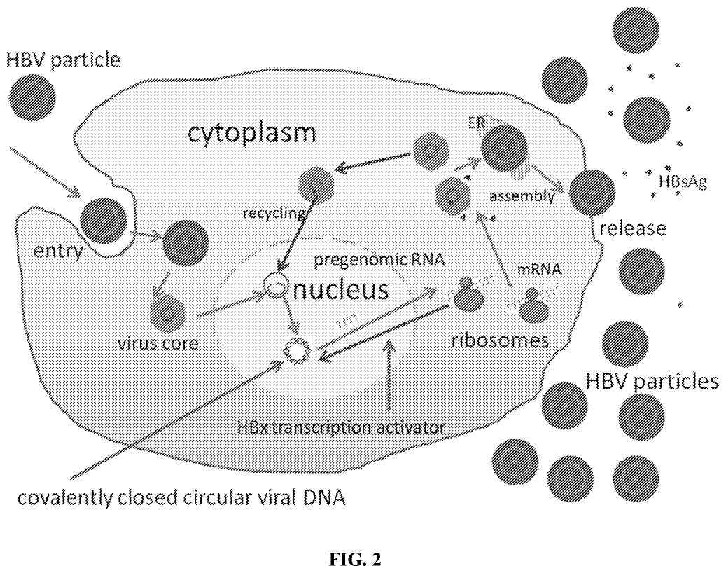

The partially double-stranded HBV genome is converted by host factors to covalently closed circular DNA (cccDNA). The cccDNA is transcribed by a host RNA polymerase to produce viral mRNA including pre-genomic RNA (pgRNA). pgRNA is reversed transcribed by the HBV polymerase into genomic HBV DNA that can be converted into cccDNA, packaged into virions, or integrated into the host cell's genome (FIG. 2). cccDNA, a key component of the HBV life cycle, is a stable molecule responsible for chronic HBV infection. Editing of the HBV genome can disrupt the formation of cccDNA, thereby reducing the pathogenicity of the virus.

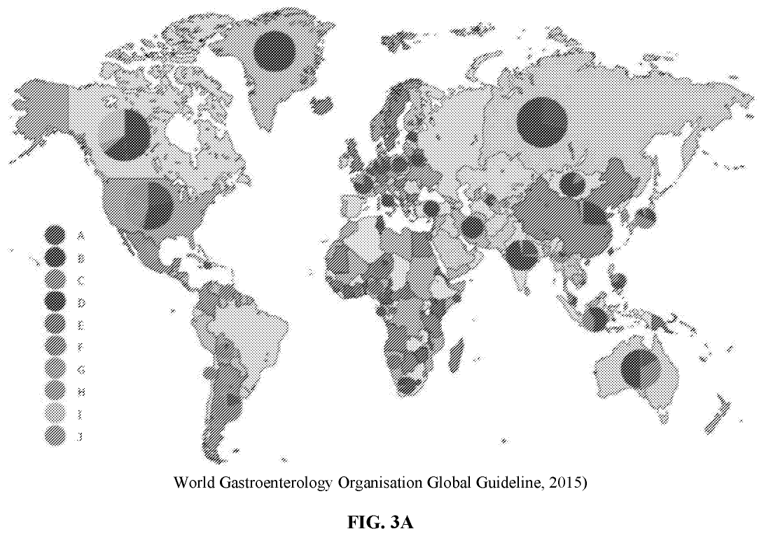

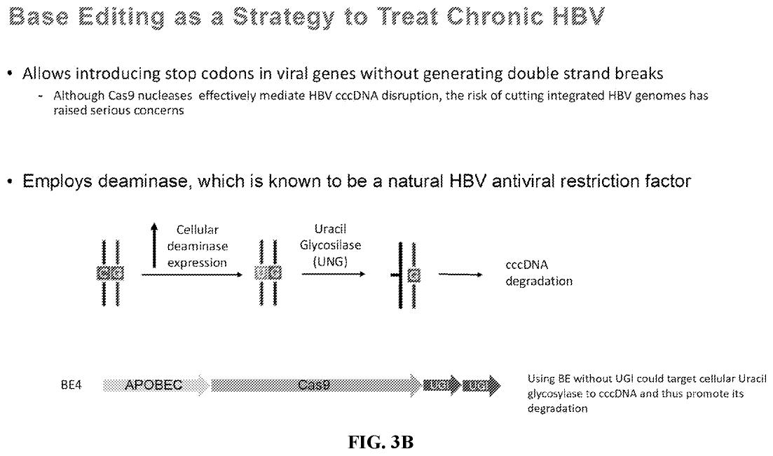

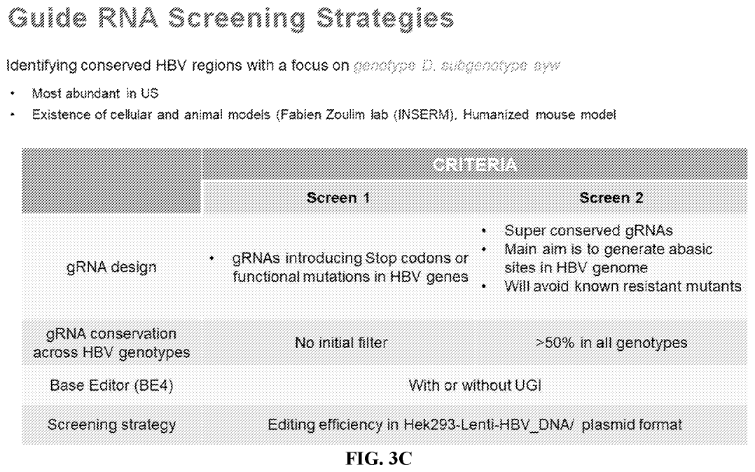

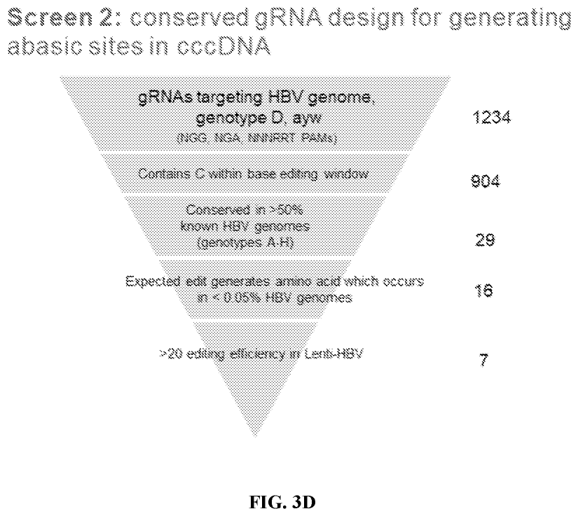

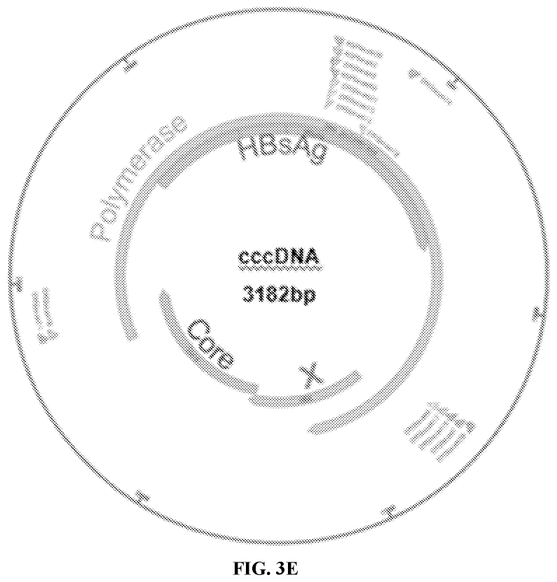

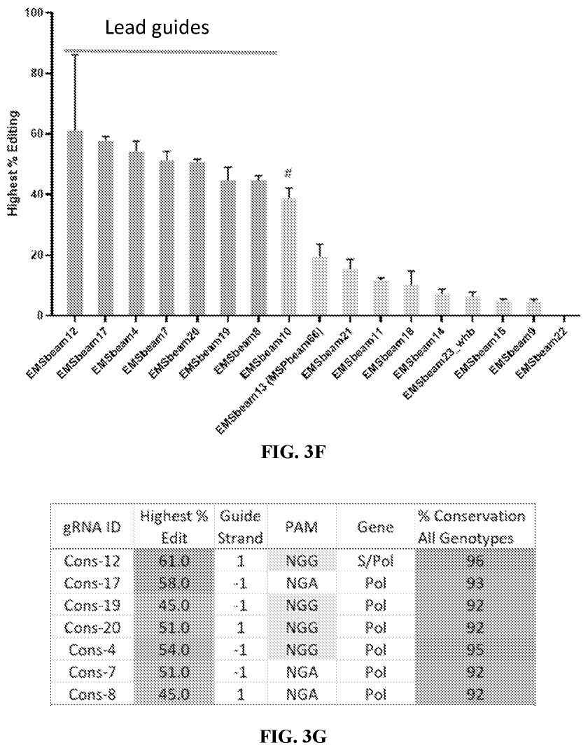

There are ten different HBV genotypes (A-J) (FIG. 3A). A “genotype” is characterized by <92% sequence identity with any other genome, and a sub-genotype is characterized by <96 to 92% sequence identity. HBV of genotype D is the most prevalent in the United States (FIG. 3A). Research models of HBV genotype D are available including viral stocks (e.g., genotype D, subgenotype ayw (Imquest)) and mouse models (e.g., humanized mouse model (Phoenixbio). Thus, in some embodiments, methods and compositions are provided that target HBV ORFs for editing. These compositions can comprise a nucleobase editor having a Cas9 or other nucleic acid programmable DNA binding protein domain and an adenosine or cytosine deaminase domain. In some embodiments, the base editor introduces one or more alterations into an HBV ORF. In some embodiments, the alteration results in a mutation in a conserved portion of an HBV protein. In particular embodiments, the alteration introduces one or more stop codons. Throughout the specification, the introduction of a stop codon, resulting in the premature termination of the protein is represented by the amino acid symbol, the amino acid position, and the term STOP (e.g., R87STOP indicates that the codon encoding Arginine at amino acid position 87 is replaced by a Stop codon). Advantageously, the methods of the present invention do not introduce double stranded breaks in the HBV genome.

The invention provides strategies for using base editing to treat chronic HBV (FIG. 3B). Described herein are screens for identifying guide RNAs that introduce stop codons or functional mutations into HBV genes or that identify gRNAs that generate abasic sites in superconserved regions of the HBV genome (FIG. 3C). Introducing stop condons into viral genes using the methods and compositions described herein can be accomplished without generating double strand breaks, thereby eliminating or reducing the risk of cutting host genetic material after HBV integrates into the host's genome. Additionally, the compositions employ a deaminase that is a natural HBV antiviral restriction factor. For example, inducing APOBEC cytodine deaminases with interferon alpha or Lymphotoxin R receptor (LTBR) promotes abasic site formation and cccDNA degradation (FIG. 3B). Furthermore, using a base editor without uracil glycosilase inhibitor domains can target cellular uracil glycosylase to cccDNA and promote its degradation.

Another screen provided identifies conserved gRNAs that can be used to generate abasic sites in cccDNA. Referring to FIG. 3D, 7 guide RNAs were identified that had greater than 20% editing efficiency when a lentivirus was used to introduce a base editor and gRNA (Lenti-HBV). The gRNAs targeting conserved regions are shown at FIG. 3E. Several gRNAs had at least 45% editing efficiency (FIGS. 3F and 3G).

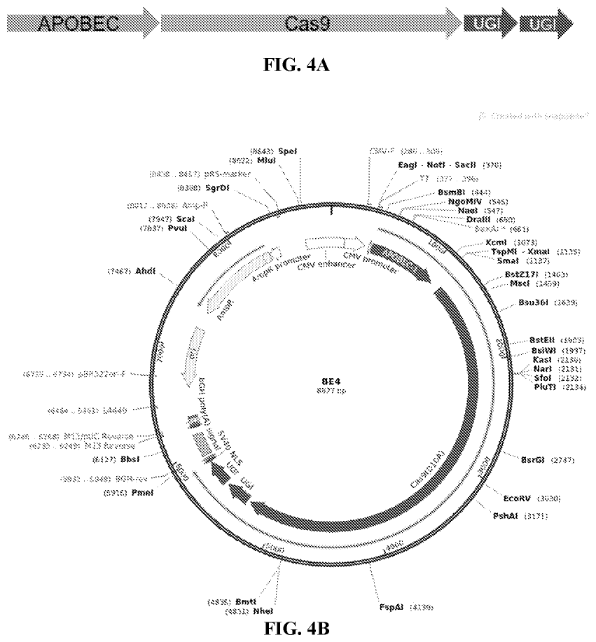

In some aspects, methods and compositions are provided for editing HBV cccDNA with a base editor comprising a cytidine deaminase or adenosine deaminase domain. In one embodiment, a base editor comprises an APOBEC cytidine deaminase domain, a Cas9 domain, and, optionally, one or more uracil glycosylase inhibitor (UGI) domains (FIGS. 4A, 4B).

### Nucleobase Editor

Disclosed herein is a base editor or a nucleobase editor for editing, modifying or altering a target nucleotide sequence of a polynucleotide. Described herein is a nucleobase editor or a base editor comprising a polynucleotide programmable nucleotide binding domain and a nucleobase editing domain (e.g., adenosine deaminase, cytidine deaminase). A polynucleotide programmable nucleotide binding domain, when in conjunction with a bound guide polynucleotide (e.g., gRNA), can specifically bind to a target polynucleotide sequence (i.e., via complementary base pairing between bases of the bound guide nucleic acid and bases of the target polynucleotide sequence) and thereby localize the base editor to the target nucleic acid sequence desired to be edited. In some embodiments, the target polynucleotide sequence comprises single-stranded DNA or double-stranded DNA. In some embodiments, the target polynucleotide sequence comprises RNA. In some embodiments, the target polynucleotide sequence comprises a DNA-RNA hybrid.

### Polynucleotide Programmable Nucleotide Binding Domain

It should be appreciated that polynucleotide programmable nucleotide binding domains can also include nucleic acid programmable proteins that bind RNA. For example, the polynucleotide programmable nucleotide binding domain can be associated with a nucleic acid that guides the polynucleotide programmable nucleotide binding domain to an RNA. Other nucleic acid programmable DNA binding proteins are also within the scope of this disclosure, though they are not specifically listed in this disclosure.

A polynucleotide programmable nucleotide binding domain of a base editor can itself comprise one or more domains. For example, a polynucleotide programmable nucleotide binding domain can comprise one or more nuclease domains. In some embodiments, the nuclease domain of a polynucleotide programmable nucleotide binding domain can comprise an endonuclease or an exonuclease. Herein the term “exonuclease” refers to a protein or polypeptide capable of digesting a nucleic acid (e.g., RNA or DNA) from free ends, and the term “endonuclease” refers to a protein or polypeptide capable of catalyzing (e.g., cleaving) internal regions in a nucleic acid (e.g., DNA or RNA). In some embodiments, an endonuclease can cleave a single strand of a double-stranded nucleic acid. In some embodiments, an endonuclease can cleave both strands of a double-stranded nucleic acid molecule. In some embodiments a polynucleotide programmable nucleotide binding domain can be a deoxyribonuclease. In some embodiments a polynucleotide programmable nucleotide binding domain can be a ribonuclease.

In some embodiments, a nuclease domain of a polynucleotide programmable nucleotide binding domain can cut zero, one, or two strands of a target polynucleotide. In some cases, the polynucleotide programmable nucleotide binding domain can comprise a nickase domain. Herein the term “nickase” refers to a polynucleotide programmable nucleotide binding domain comprising a nuclease domain that is capable of cleaving only one strand of the two strands in a duplexed nucleic acid molecule (e.g., DNA). In some embodiments, a nickase can be derived from a fully catalytically active (e.g., natural) form of a polynucleotide programmable nucleotide binding domain by introducing one or more mutations into the active polynucleotide programmable nucleotide binding domain. For example, where a polynucleotide programmable nucleotide binding domain comprises a nickase domain derived from Cas9, the Cas9-derived nickase domain can include a D10A mutation and a histidine at position 840. In such cases, the residue H840 retains catalytic activity and can thereby cleave a single strand of the nucleic acid duplex. In another example, a Cas9-derived nickase domain can comprise an H840A mutation, while the amino acid residue at position 10 remains a D. In some embodiments, a nickase can be derived from a fully catalytically active (e.g., natural) form of a polynucleotide programmable nucleotide binding domain by removing all or a portion of a nuclease domain that is not required for the nickase activity. For example, where a polynucleotide programmable nucleotide binding domain comprises a nickase domain derived from Cas9, the Cas9-derived nickase domain can comprise a deletion of all or a portion of the RuvC domain or the HNH domain.

The amino acid sequence of an exemplary catalytically active Cas9 is as follows:

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

A base editor comprising a polynucleotide programmable nucleotide binding domain comprising a nickase domain is thus able to generate a single-strand DNA break (nick) at a specific polynucleotide target sequence (e.g., determined by the complementary sequence of a bound guide nucleic acid). In some embodiments, the strand of a nucleic acid duplex target polynucleotide sequence that is cleaved by a base editor comprising a nickase domain (e.g., Cas9-derived nickase domain) is the strand that is not edited by the base editor (i.e., the strand that is cleaved by the base editor is opposite to a strand comprising a base to be edited). In other embodiments, a base editor comprising a nickase domain (e.g., Cas9-derived nickase domain) can cleave the strand of a DNA molecule which is being targeted for editing. In such cases, the non-targeted strand is not cleaved.

Also provided herein are base editors comprising a polynucleotide programmable nucleotide binding domain which is catalytically dead (i.e., incapable of cleaving a target polynucleotide sequence). Herein the terms “catalytically dead” and “nuclease dead” are used interchangeably to refer to a polynucleotide programmable nucleotide binding domain which has one or more mutations and/or deletions resulting in its inability to cleave a strand of a nucleic acid. In some embodiments, a catalytically dead polynucleotide programmable nucleotide binding domain base editor can lack nuclease activity as a result of specific point mutations in one or more nuclease domains. For example, in the case of a base editor comprising a Cas9 domain, the Cas9 can comprise both a D10A mutation and an H840A mutation. Such mutations inactivate both nuclease domains, thereby resulting in the loss of nuclease activity. In other embodiments, a catalytically dead polynucleotide programmable nucleotide binding domain can comprise one or more deletions of all or a portion of a catalytic domain (e.g., RuvC1 and/or HNH domains). In further embodiments, a catalytically dead polynucleotide programmable nucleotide binding domain comprises a point mutation (e.g., D10A or H840A) as well as a deletion of all or a portion of a nuclease domain.

Also contemplated herein are mutations capable of generating a catalytically dead polynucleotide programmable nucleotide binding domain from a previously functional version of the polynucleotide programmable nucleotide binding domain. For example, in the case of catalytically dead Cas9 (“dCas9”), variants having mutations other than D10A and H840A are provided, which result in nuclease inactivated Cas9. Such mutations, by way of example, include other amino acid substitutions at D10 and H840, or other substitutions within the nuclease domains of Cas9 (e.g., substitutions in the HNH nuclease subdomain and/or the RuvC1 subdomain). Additional suitable nuclease-inactive dCas9 domains can be apparent to those of skill in the art based on this disclosure and knowledge in the field, and are within the scope of this disclosure. Such additional exemplary suitable nuclease-inactive Cas9 domains include, but are not limited to, D10A/H840A, D10A/D839A/H840A, and D10A/D839A/H840A/N863A mutant domains (See, e.g., Prashant et al., CAS9 transcriptional activators for target specificity screening and paired nickases for cooperative genome engineering. Nature Biotechnology. 2013; 31(9): 833-838, the entire contents of which are incorporated herein by reference).

Non-limiting examples of a polynucleotide programmable nucleotide binding domain which can be incorporated into a base editor include a CRISPR protein-derived domain, a restriction nuclease, a meganuclease, TAL nuclease (TALEN), and a zinc finger nuclease (ZFN). In some cases, a base editor comprises a polynucleotide programmable nucleotide binding domain comprising a natural or modified protein or portion thereof which via a bound guide nucleic acid is capable of binding to a nucleic acid sequence during CRISPR (i.e., Clustered Regularly Interspaced Short Palindromic Repeats)-mediated modification of a nucleic acid. Such a protein is referred to herein as a “CRISPR protein”. Accordingly, disclosed herein is a base editor comprising a polynucleotide programmable nucleotide binding domain comprising all or a portion of a CRISPR protein (i.e. a base editor comprising as a domain all or a portion of a CRISPR protein, also referred to as a “CRISPR protein-derived domain” of the base editor). A CRISPR protein-derived domain incorporated into a base editor can be modified compared to a wild-type or natural version of the CRISPR protein. For example, as described below a CRISPR protein-derived domain can comprise one or more mutations, insertions, deletions, rearrangements and/or recombinations relative to a wild-type or natural version of the CRISPR protein.

CRISPR is an adaptive immune system that provides protection against mobile genetic elements (viruses, transposable elements and conjugative plasmids). CRISPR clusters contain spacers, sequences complementary to antecedent mobile elements, and target invading nucleic acids. CRISPR clusters are transcribed and processed into CRISPR RNA (crRNA). In type II CRISPR systems, correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA. Subsequently, Cas9/crRNA/tracrRNA endonucleolytically cleaves linear or circular dsDNA target complementary to the spacer. The target strand not complementary to crRNA is first cut endonucleolytically, and then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M., Chylinski K., Fonfara I., Hauer M., Doudna J. A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self-versus-non-self.

In some embodiments, the methods described herein can utilize an engineered Cas protein. A guide RNA (gRNA) is a short synthetic RNA composed of a scaffold sequence necessary for Cas-binding and a user-defined ˜20 nucleotide spacer that defines the genomic (or polynucleotide, e.g., DNA or RNA) target to be modified. Thus, a skilled artisan can change the genomic or polynucleotide target of the Cas protein by changing the target sequence present in the gRNA. The specificity of the Cas protein is partially determined by how specific the gRNA targeting sequence is for the genomic polynucleotide target sequence compared to the rest of the genome.

In some embodiments, the gRNA scaffold sequence is as follows: GUUUUAGAGC UAGAAAUAGC AAGUUAAAAU AAGGCUAGUC CGUUAUCAAC UUGAAAAAGU GGCACCGAGU CGGUGCUUUU.

In an embodiment, the RNA scaffold comprises a stem loop. In an embodiment, the RNA scaffold comprises the nucleic acid sequence:

GUUUUUGUACUCUCAAGAUUUAAGUAACUGUACAACGAAACUUACACAGU
UACUUAAAUCUUGCAGAAGCUACAAAGAUAAGGCUUCAUGCCGAAAUCAA
CACCCUGUCAUUUUAUGGCAGGGU

G. In an embodiment, the RNA scaffold comprises the nucleic acid sequence:

GUUUUAGAGCUAGAAAUAGCAAGUUAAAAUAAGGCUAGUCCGUUAUCAAC
UUGAAAAAGUGGCACCGAGUCGGUGCUUUU.

In an embodiment, an S. pyrogenes sgRNA scaffold polynucleotide sequence is as follows:

GUUUUAGAGCUAGAAAUAGCAAGUUAAAAUAAGGCUAGUCCGUUAUCAAC
UUGAAAAAGUGGCACCGAGUCGGUGC.

In an embodiment, an S. aureus sgRNA scaffold polynucleotide sequence is as follows:

GUUUUAGUACUCUGUAAUGAAAAUUACAGAAUCUACUAAAACAAGGCAAA
AUGCCGUGUUUAUCUCGUCAACUUGUUGGCGAGA.

In an embodiment, a BhCas12b sgRNA scaffold has the following polynucleotide sequence:

GUUCUGTCUUUUGGUCAGGACAACCGUCUAGCUAUAAGUGCUGCAGGGUG
UGAGAAACUCCUAUUGCUGGACGAUGUCUCUUACGAGGCAUUAGCAC.

In an embodiment, a BvCas12b sgRNA scaffold has the following polynucleotide sequence:

GACCUAUAGGGUCAAUGAAUCUGUGCGUGUGCCAUAAGUAAUUAAAAAUU
ACCCACCACAGGAGCACCUGAAAACAGGUGCUUGGCAC.

In some embodiments, a CRISPR protein-derived domain incorporated into a base editor is an endonuclease (e.g., deoxyribonuclease or ribonuclease) capable of binding a target polynucleotide when in conjunction with a bound guide nucleic acid. In some embodiments, a CRISPR protein-derived domain incorporated into a base editor is a nickase capable of binding a target polynucleotide when in conjunction with a bound guide nucleic acid. In some embodiments, a CRISPR protein-derived domain incorporated into a base editor is a catalytically dead domain capable of binding a target polynucleotide when in conjunction with a bound guide nucleic acid. In some embodiments, a target polynucleotide bound by a CRISPR protein derived domain of a base editor is DNA. In some embodiments, a target polynucleotide bound by a CRISPR protein-derived domain of a base editor is RNA. Cas proteins that can be used herein include class 1 and class 2. Non-limiting examples of Cas proteins include Cas1, Cas1B, Cas2, Cas3, Cas4, Cas5, Cas5d, Cas5t, Cas5h, Cas5a, Cas6, Cas7, Cas8, Cas9 (also known as Csn1 or Csx12), Cas10, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csf1, Csf2, CsO, Csf4, Csd1, Csd2, Cst1, Cst2, Csh1, Csh2, Csa1, Csa2, Csa3, Csa4, Csa5, Cas12a/Cpf1, Cas12b/C2c1, Cas12c/C2c3, Cas12d/CasY, Cas12e/CasX, Cas12g, Cas12h, and Cas12i, CARF, DinG, homologues thereof, or modified versions thereof. An unmodified CRISPR enzyme can have DNA cleavage activity, such as Cas9, which has two functional endonuclease domains: RuvC and HNH. A CRISPR enzyme can direct cleavage of one or both strands at a target sequence, such as within a target sequence and/or within a complement of a target sequence. For example, a CRISPR enzyme can direct cleavage of one or both strands within about 1, 2, 3, 4, 5, 6, 7, 8, 9, 10, 15, 20, 25, 50, 100, 200, 500, or more base pairs from the first or last nucleotide of a target sequence.

A vector that encodes a CRISPR enzyme that is mutated to with respect, to a corresponding wild-type enzyme such that the mutated CRISPR enzyme lacks the ability to cleave one or both strands of a target polynucleotide containing a target sequence can be used. Cas9 can refer to a polypeptide with at least or at least about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., Cas9 from S. pyogenes). Cas9 can refer to a polypeptide with at most or at most about 50%, 60%, 70%, 80%, 90%, 91%, 92%, 93%, 94%, 95%, 96%, 97%, 98%, 99%, or 100% sequence identity and/or sequence homology to a wild type exemplary Cas9 polypeptide (e.g., from S. pyogenes). Cas9 can refer to the wild type or a modified form of the Cas9 protein that can comprise an amino acid change such as a deletion, insertion, substitution, variant, mutation, fusion, chimera, or any combination thereof.

In some embodiments, a CRISPR protein-derived domain of a base editor can include all or a portion of Cas9 from Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); Neisseria meningitidis (NCBI Ref: YP_002342100.1), Streptococcus pyogenes, or Staphylococcus aureus.

### Cas9 Domains of Nucleobase Editors

Cas9 nuclease sequences and structures are well known to those of skill in the art (See, e.g., “Complete genome sequence of an M1 strain of Streptococcus pyogenes.” Ferretti et al., J. J., McShan W. M., Ajdic D. J., Savic D. J., Savic G., Lyon K., Primeaux C, Sezate S., Suvorov A. N., Kenton S., Lai H. S., Lin S. P., Qian Y., Jia H. G., Najar F. Z., Ren Q., Zhu H., Song L., White J., Yuan X., Clifton S. W., Roe B. A., McLaughlin R. E., Proc. Natl. Acad. Sci. U.S.A. 98:4658-4663(2001); “CRISPR RNA maturation by trans-encoded small RNA and host factor RNase III.” Deltcheva E., Chylinski K., Sharma C. M., Gonzales K., Chao Y., Pirzada Z. A., Eckert M. R., Vogel J., Charpentier E., Nature 471:602-607(2011); and “A programmable dual-RNA-guided DNA endonuclease in adaptive bacterial immunity.” Jinek M., Chylinski K., Fonfara I., Hauer M., Doudna J. A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

In some aspects, a nucleic acid programmable DNA binding protein (napDNAbp) is a Cas9 domain. Non-limiting, exemplary Cas9 domains are provided herein. The Cas9 domain may be a nuclease active Cas9 domain, a nuclease inactive Cas9 domain, or a Cas9 nickase. In some embodiments, the Cas9 domain is a nuclease active domain. For example, the Cas9 domain may be a Cas9 domain that cuts both strands of a duplexed nucleic acid (e.g., both strands of a duplexed DNA molecule). In some embodiments, the Cas9 domain comprises any one of the amino acid sequences as set forth herein. In some embodiments the Cas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth herein. In some embodiments, the Cas9 domain comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more mutations compared to any one of the amino acid sequences set forth herein. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, proteins comprising fragments of Cas9 are provided. For example, in some embodiments, a protein comprises one of two Cas9 domains: (1) the gRNA binding domain of Cas9; or (2) the DNA cleavage domain of Cas9. In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to wild type Cas9. In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9. In some embodiments, the fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, Cas9 fusion proteins as provided herein comprise the full-length amino acid sequence of a Cas9 protein, e.g., one of the Cas9 sequences provided herein. In other embodiments, however, fusion proteins as provided herein do not comprise a full-length Cas9 sequence, but only one or more fragments thereof. Exemplary amino acid sequences of suitable Cas9 domains and Cas9 fragments are provided herein, and additional suitable sequences of Cas9 domains and fragments will be apparent to those of skill in the art.

A Cas9 protein can associate with a guide RNA that guides the Cas9 protein to a specific DNA sequence that has complementary to the guide RNA. In some embodiments, the polynucleotide programmable nucleotide binding domain is a Cas9 domain, for example a nuclease active Cas9, a Cas9 nickase (nCas9), or a nuclease inactive Cas9 (dCas9). Examples of nucleic acid programmable DNA binding proteins include, without limitation, Cas9 (e.g., dCas9 and nCas9), CasX, CasY, Cpf1, Cas12b/C2C1, and Cas12c/C2C3.

In some embodiments, wild type Cas9 corresponds to Cas9 from Streptococcus pyogenes (NCBI Reference Sequence: NC_017053.1, nucleotide and amino acid sequences as follows).

ATGGATAAGAAATACTCAATAGGCTTAGATATCGGCACAAATAGCGTCGGATGGGCGGTGAT
CACTGATGATTATAAGGTTCCGTCTAAAAAGTTCAAGGTTCTGGGAAATACAGACCGCCACA
GTATCAAAAAAAATCTTATAGGGGCTCTTTTATTTGGCAGTGGAGAGACAGCGGAAGCGACT
CGTCTCAAACGGACAGCTCGTAGAAGGTATACACGTCGGAAGAATCGTATTTGTTATCTACA
GGAGATTTTTTCAAATGAGATGGCGAAAGTAGATGATAGTTTCTTTCATCGACTTGAAGAGT
CTTTTTTGGTGGAAGAAGACAAGAAGCATGAACGTCATCCTATTTTTGGAAATATAGTAGAT
GAAGTTGCTTATCATGAGAAATATCCAACTATCTATCATCTGCGAAAAAAATTGGCAGATIC
TACTGATAAAGCGGATTTGCGCTTAATCTATTTGGCCTTAGCGCATATGATTAAGTTTCGTG
GTCATTTTTTGATTGAGGGAGATTTAAATCCTGATAATAGTGATGTGGACAAACTATTTATC
CAGTTGGTACAAATCTACAATCAATTATTTGAAGAAAACCCTATTAACGCAAGTAGAGTAGA
TGCTAAAGCGATTCTTTCTGCACGATTGAGTAAATCAAGACGATTAGAAAATCTCATTGCTC
AGCTCCCCGGTGAGAAGAGAAATGGCTTGTTTGGGAATCTCATTGCTTTGTCATTGGGATTG
ACCCCTAATTTTAAATCAAATTTTGATTTGGCAGAAGATGCTAAATTACAGCTTTCAAAAGA
TACTTACGATGATGATTTAGATAATTTATTGGCGCAAATTGGAGATCAATATGCTGATTTGT
TTTTGGCAGCTAAGAATTTATCAGATGCTATTTTACTTTCAGATATCCTAAGAGTAAATAGT
GAAATAACTAAGGCTCCCCTATCAGCTTCAATGATTAAGCGCTACGATGAACATCATCAAGA
CTTGACTCTTTTAAAAGCTTTAGTTCGACAACAACTTCCAGAAAAGTATAAAGAAATCTTTT
TTGATCAATCAAAAAACGGATATGCAGGTTATATTGATGGGGGAGCTAGCCAAGAAGAATTT
TATAAATTTATCAAACCAATTTTAGAAAAAATGGATGGTACTGAGGAATTATTGGTGAAACT
AAATCGTGAAGATTTGCTGCGCAAGCAACGGACCTTTGACAACGGCTCTATTCCCCATCAAA
TTCACTTGGGTGAGCTGCATGCTATTTTGAGAAGACAAGAAGACTTTTATCCATTTTTAAAA
GACAATCGTGAGAAGATTGAAAAAATCTTGACTTTTCGAATTCCTTATTATGTTGGTCCATT
GGCGCGTGGCAATAGTCGTTTTGCATGGATGACTCGGAAGTCTGAAGAAACAATTACCCCAT
GGAATTTTGAAGAAGTTGTCGATAAAGGTGCTTCAGCTCAATCATTTATTGAACGCATGACA
AACTTTGATAAAAATCTTCCAAATGAAAAAGTACTACCAAAACATAGTTTGCTTTATGAGTA
TTTTACGGTTTATAACGAATTGACAAAGGTCAAATATGTTACTGAGGGAATGCGAAAACCAG
CATTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTTGA
AATTTCAGGAGTTGAAGATAGATTTAATGCTTCATTAGGCGCCTACCATGATTTGCTAAAAA
TTATTAAAGATAAAGATTTTTTGGATAATGAAGAAAATGAAGATATCTTAGAGGATATTGTT
TTAACATTGACCTTATTTGAAGATAGGGGGATGATTGAGGAAAGACTTAAAACATATGCTCA
CCTCTTTGATGATAAGGTGATGAAACAGCTTAAACGTCGCCGTTATACTGGTTGGGGACGTT
TGTCTCGAAAATTGATTAATGGTATTAGGGATAAGCAATCTGGCAAAACAATATTAGATTTT
TTGAAATCAGATGGTTTTGCCAATCGCAATTTTATGCAGCTGATCCATGATGATAGTTTGAC
ATTTAAAGAAGATATTCAAAAAGCACAGGTGTCTGGACAAGGCCATAGTTTACATGAACAGA
TTGCTAACTTAGCTGGCAGTCCTGCTATTAAAAAAGGTATTTTACAGACTGTAAAAATTGTT
GATGAACTGGTCAAAGTAATGGGGCATAAGCCAGAAAATATCGTTATTGAAATGGCACGTGA
AAATCAGACAACTCAAAAGGGCCAGAAAAATTCGCGAGAGCGTATGAAACGAATCGAAGAAG
GTATCAAAGAATTAGGAAGTCAGATTCTTAAAGAGCATCCTGTTGAAAATACTCAATTGCAA
AATGAAAAGCTCTATCTCTATTATCTACAAAATGGAAGAGACATGTATGTGGACCAAGAATT
AGATATTAATCGTTTAAGTGATTATGATGTCGATCACATTGTTCCACAAAGTTTCATTAAAG
ACGATTCAATAGACAATAAGGTACTAACGCGTTCTGATAAAAATCGTGGTAAATCGGATAAC
GTTCCAAGTGAAGAAGTAGTCAAAAAGATGAAAAACTATTGGAGACAACTTCTAAACGCCAA
GTTAATCACTCAACGTAAGTTTGATAATTTAACGAAAGCTGAACGTGGAGGTTTGAGTGAAC
TTGATAAAGCTGGTTTTATCAAACGCCAATTGGTTGAAACTCGCCAAATCACTAAGCATGTG
GCACAAATTTTGGATAGTCGCATGAATACTAAATACGATGAAAATGATAAACTTATTCGAGA
GGTTAAAGTGATTACCTTAAAATCTAAATTAGTTTCTGACTTCCGAAAAGATTTCCAATTCT
ATAAAGTACGTGAGATTAACAATTACCATCATGCCCATGATGCGTATCTAAATGCCGTCGTT
GGAACTGCTTTGATTAAGAAATATCCAAAACTTGAATCGGAGTTTGTCTATGGTGATTATAA
AGTTTATGATGTTCGTAAAATGATTGCTAAGTCTGAGCAAGAAATAGGCAAAGCAACCGCAA
AATATTTCTTTTACTCTAATATCATGAACTTCTTCAAAACAGAAATTACACTTGCAAATGGA
GAGATTCGCAAACGCCCTCTAATCGAAACTAATGGGGAAACTGGAGAAATTGTCTGGGATAA
AGGGCGAGATTTTGCCACAGTGCGCAAAGTATTGTCCATGCCCCAAGTCAATATTGTCAAGA
AAACAGAAGTACAGACAGGCGGATTCTCCAAGGAGTCAATTTTACCAAAAAGAAATTCGGAC
AAGCTTATTGCTCGTAAAAAAGACTGGGATCCAAAAAAATATGGTGGTTTTGATAGTCCAAC
GGTAGCTTATTCAGTCCTAGTGGTTGCTAAGGTGGAAAAAGGGAAATCGAAGAAGTTAAAAT
CCGTTAAAGAGTTACTAGGGATCACAATTATGGAAAGAAGTTCCTTTGAAAAAAATCCGATT
GACTTTTTAGAAGCTAAAGGATATAAGGAAGTTAAAAAAGACTTAATCATTAAACTACCTAA
ATATAGTCTTTTTGAGTTAGAAAACGGTCGTAAACGGATGCTGGCTAGTGCCGGAGAATTAC
AAAAAGGAAATGAGCTGGCTCTGCCAAGCAAATATGTGAATTTTTTATATTTAGCTAGTCAT
TATGAAAAGTTGAAGGGTAGTCCAGAAGATAACGAACAAAAACAATTGTTTGTGGAGCAGCA
TAAGCATTATTTAGATGAGATTATTGAGCAAATCAGTGAATTTTCTAAGCGTGTTATTTTAG
CAGATGCCAATTTAGATAAAGTTCTTAGTGCATATAACAAACATAGAGACAAACCAATACGT
GAACAAGCAGAAAATATTATTCATTTATTTACGTTGACGAATCTTGGAGCTCCCGCTGCTTT
TAAATATTTTGATACAACAATTGATCGTAAACGATATACGTCTACAAAAGAAGTTTTAGATG
CCACTCTTATCCATCAATCCATCACTGGTCTTTATGAAACACGCATTGATTTGAGTCAGCTA
GGAGGTGACTGA
MDKKYSIGLDIGTNSVGWAVITDDYKVPSKKFKVLGNTDRHSIKKNLIGALLFGSGETAEAT
RLKRTARRRYTRRKNRICYLQEIFSNEMAKVDDSFFHRLEESFLVEEDKKHERHPIFGNIVD
EVAYHEKYPTIYHLRKKLADSTDKADLRLIYLALAHMIKFRGHFLIEGDLNPDNSDVDKLFI
QLVQIYNQLFEENPINASRVDAKAILSARLSKSRRLENLIAQLPGEKRNGLFGNLIALSLGL
TPNFKSNFDLAEDAKLQLSKDTYDDDLDNLLAQIGDQYADLFLAAKNLSDAILLSDILRVNS
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGAYHDLLKIIKDKDFLDNEENEDILEDIV
LTLTLFEDRGMIEERLKTYAHLFDDKVMKQLKRRRYTGWGRLSRKLINGIRDKQSGKTILDF
LKSDGFANRNFMQLIHDDSLTFKEDIQKAQVSGQGHSLHEQIANLAGSPAIKKGILQTVKIV
DELVKVMGHKPENIVIEMARENQTTQKGQKNSRERMKRIEEGIKELGSQILKEHPVENTQLQ
NEKLYLYYLQNGRDMYVDQELDINRLSDYDVDHIVPQSFIKDDSIDNKVLTRSDKNRGKSDN
VPSEEVVKKMKNYWRQLLNAKLITQRKFDNLTKAERGGLSELDKAGFIKRQLVETRQITKHV
AQILDSRMNTKYDENDKLIREVKVITLKSKLVSDFRKDFQFYKVREINNYHHAHDAYLNAVV
GTALIKKYPKLESEFVYGDYKVYDVRKMIAKSEQEIGKATAKYFFYSNIMNFFKTEITLANG
EIRKRPLIETNGETGEIVWDKGRDFATVRKVLSMPQVNIVKKTEVQTGGFSKESILPKRNSD
KLIARKKDWDPKKYGGFDSPTVAYSVLVVAKVEKGKSKKLKSVKELLGITIMERSSFEKNPI
DFLEAKGYKEVKKDLIIKLPKYSLFELENGRKRMLASAGELQKGNELALPSKYVNFLYLASH
YEKLKGSPEDNEQKQLFVEQHKHYLDEIIEQISEFSKRVILADANLDKVLSAYNKHRDKPIR
EQAENIIHLFTLTNLGAPAAFKYFDTTIDRKRYTSTKEVLDATLIHQSITGLYETRIDLSQL
GGD
(single underline: HNH domain; double underline: RuvC domain)

In some embodiments, wild type Cas9 corresponds to, or comprises the following nucleotide and/or amino acid sequences:

ATGGATAAAAAGTATTCTATTGGTTTAGACATCGGCACTAATTCCGTTGGATGGGCTGTCAT
AACCGATGAATACAAAGTACCTTCAAAGAAATTTAAGGTGTTGGGGAACACAGACCGTCATT
CGATTAAAAAGAATCTTATCGGTGCCCTCCTATTCGATAGTGGCGAAACGGCAGAGGCGACT
CGCCTGAAACGAACCGCTCGGAGAAGGTATACACGTCGCAAGAACCGAATATGTTACTTACA
AGAAATTTTTAGCAATGAGATGGCCAAAGTTGACGATTCTTTCTTTCACCGTTTGGAAGAGT
CCTTCCTTGTCGAAGAGGACAAGAAACATGAACGGCACCCCATCTTTGGAAACATAGTAGAT
GAGGTGGCATATCATGAAAAGTACCCAACGATTTATCACCTCAGAAAAAAGCTAGTTGACTC
AACTGATAAAGCGGACCTGAGGTTAATCTACTTGGCTCTTGCCCATATGATAAAGTTCCGTG
GGCACTTTCTCATTGAGGGTGATCTAAATCCGGACAACTCGGATGTCGACAAACTGTTCATC
CAGTTAGTACAAACCTATAATCAGTTGTTTGAAGAGAACCCTATAAATGCAAGTGGCGTGGA
TGCGAAGGCTATTCTTAGCGCCCGCCTCTCTAAATCCCGACGGCTAGAAAACCTGATCGCAC
AATTACCCGGAGAGAAGAAAAATGGGTTGTTCGGTAACCTTATAGCGCTCTCACTAGGCCTG
ACACCAAATTTTAAGTCGAACTTCGACTTAGCTGAAGATGCCAAATTGCAGCTTAGTAAGGA
CACGTACGATGACGATCTCGACAATCTACTGGCACAAATTGGAGATCAGTATGCGGACTTAT
TTTTGGCTGCCAAAAACCTTAGCGATGCAATCCTCCTATCTGACATACTGAGAGTTAATACT
GAGATTACCAAGGCGCCGTTATCCGCTTCAATGATCAAAAGGTACGATGAACATCACCAAGA
CTTGACACTTCTCAAGGCCCTAGTCCGTCAGCAACTGCCTGAGAAATATAAGGAAATATTCT
TTGATCAGTCGAAAAACGGGTACGCAGGTTATATTGACGGCGGAGCGAGTCAAGAGGAATTC
TACAAGTTTATCAAACCCATATTAGAGAAGATGGATGGGACGGAAGAGTTGCTTGTAAAACT
CAATCGCGAAGATCTACTGCGAAAGCAGCGGACTTTCGACAACGGTAGCATTCCACATCAAA
TCCACTTAGGCGAATTGCATGCTATACTTAGAAGGCAGGAGGATTTTTATCCGTTCCTCAAA
GACAATCGTGAAAAGATTGAGAAAATCCTAACCTTTCGCATACCTTACTATGTGGGACCCCT
GGCCCGAGGGAACTCTCGGTTCGCATGGATGACAAGAAAGTCCGAAGAAACGATTACTCCAT
GGAATTTTGAGGAAGTTGTCGATAAAGGTGCGTCAGCTCAATCGTTCATCGAGAGGATGACC
AACTTTGACAAGAATTTACCGAACGAAAAAGTATTGCCTAAGCACAGTTTACTTTACGAGTA
TTTCACAGTGTACAATGAACTCACGAAAGTTAAGTATGTCACTGAGGGCATGCGTAAACCCG
CCTTTCTAAGCGGAGAACAGAAGAAAGCAATAGTAGATCTGTTATTCAAGACCAACCGCAAA
GTGACAGTTAAGCAATTGAAAGAGGACTACTTTAAGAAAATTGAATGCTTCGATTCTGTCGA
GATCTCCGGGGTAGAAGATCGATTTAATGCGTCACTTGGTACGTATCATGACCTCCTAAAGA
TAATTAAAGATAAGGACTTCCTGGATAACGAAGAGAATGAAGATATCTTAGAAGATATAGTG
TTGACTCTTACCCTCTTTGAAGATCGGGAAATGATTGAGGAAAGACTAAAAACATACGCTCA
CCTGTTCGACGATAAGGTTATGAAACAGTTAAAGAGGCGTCGCTATACGGGCTGGGGACGAT
TGTCGCGGAAACTTATCAACGGGATAAGAGACAAGCAAAGTGGTAAAACTATTCTCGATTTT
CTAAAGAGCGACGGCTTCGCCAATAGGAACTTTATGCAGCTGATCCATGATGACTCTTTAAC
CTTCAAAGAGGATATACAAAAGGCACAGGTTTCCGGACAAGGGGACTCATTGCACGAACATA
TTGCGAATCTTGCTGGTTCGCCAGCCATCAAAAAGGGCATACTCCAGACAGTCAAAGTAGTG
GATGAGCTAGTTAAGGTCATGGGACGTCACAAACCGGAAAACATTGTAATCGAGATGGCACG
CGAAAATCAAACGACTCAGAAGGGGCAAAAAAACAGTCGAGAGCGGATGAAGAGAATAGAAG
AGGGTATTAAAGAACTGGGCAGCCAGATCTTAAAGGAGCATCCTGTGGAAAATACCCAATTG
CAGAACGAGAAACTTTACCTCTATTACCTACAAAATGGAAGGGACATGTATGTTGATCAGGA
ACTGGACATAAACCGTTTATCTGATTACGACGTCGATCACATTGTACCCCAATCCTTTTTGA
AGGACGATTCAATCGACAATAAAGTGCTTACACGCTCGGATAAGAACCGAGGGAAAAGTGAC
AATGTTCCAAGCGAGGAAGTCGTAAAGAAAATGAAGAACTATTGGCGGCAGCTCCTAAATGC
GAAACTGATAACGCAAAGAAAGTTCGATAACTTAACTAAAGCTGAGAGGGGTGGCTTGTCTG
AACTTGACAAGGCCGGATTTATTAAACGTCAGCTCGTGGAAACCCGCCAAATCACAAAGCAT
GTTGCACAGATACTAGATTCCCGAATGAATACGAAATACGACGAGAACGATAAGCTGATTCG
GGAAGTCAAAGTAATCACTTTAAAGTCAAAATTGGTGTCGGACTTCAGAAAGGATTTTCAAT
TCTATAAAGTTAGGGAGATAAATAACTACCACCATGCGCACGACGCTTATCTTAATGCCGTC
GTAGGGACCGCACTCATTAAGAAATACCCGAAGCTAGAAAGTGAGTTTGTGTATGGTGATTA
CAAAGTTTATGACGTCCGTAAGATGATCGCGAAAAGCGAACAGGAGATAGGCAAGGCTACAG
CCAAATACTTCTTTTATTCTAACATTATGAATTTCTTTAAGACGGAAATCACTCTGGCAAAC
GGAGAGATACGCAAACGACCTTTAATTGAAACCAATGGGGAGACAGGTGAAATCGTATGGGA
TAAGGGCCGGGACTTCGCGACGGTGAGAAAAGTTTTGTCCATGCCCCAAGTCAACATAGTAA
AGAAAACTGAGGTGCAGACCGGAGGGTTTTCAAAGGAATCGATTCTTCCAAAAAGGAATAGT
GATAAGCTCATCGCTCGTAAAAAGGACTGGGACCCGAAAAAGTACGGTGGCTTCGATAGCCC
TACAGTTGCCTATTCTGTCCTAGTAGTGGCAAAAGTTGAGAAGGGAAAATCCAAGAAACTGA
AGTCAGTCAAAGAATTATTGGGGATAACGATTATGGAGCGCTCGTCTTTTGAAAAGAACCCC
ATCGACTTCCTTGAGGCGAAAGGTTACAAGGAAGTAAAAAAGGATCTCATAATTAAACTACC
AAAGTATAGTCTGTTTGAGTTAGAAAATGGCCGAAAACGGATGTTGGCTAGCGCCGGAGAGC
TTCAAAAGGGGAACGAACTCGCACTACCGTCTAAATACGTGAATTTCCTGTATTTAGCGTCC
CATTACGAGAAGTTGAAAGGTTCACCTGAAGATAACGAACAGAAGCAACTTTTTGTTGAGCA
GCACAAACATTATCTCGACGAAATCATAGAGCAAATTTCGGAATTCAGTAAGAGAGTCATCC
TAGCTGATGCCAATCTGGACAAAGTATTAAGCGCATACAACAAGCACAGGGATAAACCCATA
CGTGAGCAGGCGGAAAATATTATCCATTTGTTTACTCTTACCAACCTCGGCGCTCCAGCCGC
ATTCAAGTATTTTGACACAACGATAGATCGCAAACGATACACTTCTACCAAGGAGGTGCTAG
ACGCGACACTGATTCACCAATCCATCACGGGATTATATGAAACTCGGATAGATTTGTCACAG
CTTGGGGGTGACGGATCCCCCAAGAAGAAGAGGAAAGTCTCGAGCGACTACAAAGACCATGA
CGGTGATTATAAAGATCATGACATCGATTACAAGGATGACGATGACAAGGCTGCAGGA
MDKKYSIGLAIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
(single underline: HNH domain; double underline: RuvC domain).

In some embodiments, wild type Cas9 corresponds to Cas9 from Streptococcus pyogenes (NCBI Reference Sequence: NC_002737.2 (nucleotide sequence as follows); and Uniprot Reference Sequence: Q99ZW2 (amino acid sequence as follows):

ATGGATAAGAAATACTCAATAGGCTTAGATATCGGCACAAATAGCGTCGGATGGGCGGTGAT
CACTGATGAATATAAGGTTCCGTCTAAAAAGTTCAAGGTTCTGGGAAATACAGACCGCCACA
GTATCAAAAAAAATCTTATAGGGGCTCTTTTATTTGACAGTGGAGAGACAGCGGAAGCGACT
CGTCTCAAACGGACAGCTCGTAGAAGGTATACACGTCGGAAGAATCGTATTTGTTATCTACA
GGAGATTTTTTCAAATGAGATGGCGAAAGTAGATGATAGTTTCTTTCATCGACTTGAAGAGT
CTTTTTTGGTGGAAGAAGACAAGAAGCATGAACGTCATCCTATTTTTGGAAATATAGTAGAT
GAAGTTGCTTATCATGAGAAATATCCAACTATCTATCATCTGCGAAAAAAATTGGTAGATTC
TACTGATAAAGCGGATTTGCGCTTAATCTATTTGGCCTTAGCGCATATGATTAAGTTTCGTG
GTCATTTTTTGATTGAGGGAGATTTAAATCCTGATAATAGTGATGTGGACAAACTATTTATC
CAGTTGGTACAAACCTACAATCAATTATTTGAAGAAAACCCTATTAACGCAAGTGGAGTAGA
TGCTAAAGCGATTCTTTCTGCACGATTGAGTAAATCAAGACGATTAGAAAATCTCATTGCTC
AGCTCCCCGGTGAGAAGAAAAATGGCTTATTTGGGAATCTCATTGCTTTGTCATTGGGTTTG
ACCCCTAATTTTAAATCAAATTTTGATTTGGCAGAAGATGCTAAATTACAGCTTTCAAAAGA
TACTTACGATGATGATTTAGATAATTTATTGGCGCAAATTGGAGATCAATATGCTGATTTGT
TTTTGGCAGCTAAGAATTTATCAGATGCTATTTTACTTTCAGATATCCTAAGAGTAAATACT
GAAATAACTAAGGCTCCCCTATCAGCTTCAATGATTAAACGCTACGATGAACATCATCAAGA
CTTGACTCTTTTAAAAGCTTTAGTTCGACAACAACTTCCAGAAAAGTATAAAGAAATCTTTT
TTGATCAATCAAAAAACGGATATGCAGGTTATATTGATGGGGGAGCTAGCCAAGAAGAATTT
TATAAATTTATCAAACCAATTTTAGAAAAAATGGATGGTACTGAGGAATTATTGGTGAAACT
AAATCGTGAAGATTTGCTGCGCAAGCAACGGACCTTTGACAACGGCTCTATTCCCCATCAAA
TTCACTTGGGTGAGCTGCATGCTATTTTGAGAAGACAAGAAGACTTTTATCCATTTTTAAAA
GACAATCGTGAGAAGATTGAAAAAATCTTGACTTTTCGAATTCCTTATTATGTTGGTCCATT
GGCGCGTGGCAATAGTCGTTTTGCATGGATGACTCGGAAGTCTGAAGAAACAATTACCCCAT
GGAATTTTGAAGAAGTTGTCGATAAAGGTGCTTCAGCTCAATCATTTATTGAACGCATGACA
AACTTTGATAAAAATCTTCCAAATGAAAAAGTACTACCAAAACATAGTTTGCTTTATGAGTA
TTTTACGGTTTATAACGAATTGACAAAGGTCAAATATGTTACTGAAGGAATGCGAAAACCAG
CATTTCTTTCAGGTGAACAGAAGAAAGCCATTGTTGATTTACTCTTCAAAACAAATCGAAAA
GTAACCGTTAAGCAATTAAAAGAAGATTATTTCAAAAAAATAGAATGTTTTGATAGTGTTGA
AATTTCAGGAGTTGAAGATAGATTTAATGCTTCATTAGGTACCTACCATGATTTGCTAAAAA
TTATTAAAGATAAAGATTTTTTGGATAATGAAGAAAATGAAGATATCTTAGAGGATATTGTT
TTAACATTGACCTTATTTGAAGATAGGGAGATGATTGAGGAAAGACTTAAAACATATGCTCA
CCTCTTTGATGATAAGGTGATGAAACAGCTTAAACGTCGCCGTTATACTGGTTGGGGACGTT
TGTCTCGAAAATTGATTAATGGTATTAGGGATAAGCAATCTGGCAAAACAATATTAGATTTT
TTGAAATCAGATGGTTTTGCCAATCGCAATTTTATGCAGCTGATCCATGATGATAGTTTGAC
ATTTAAAGAAGACATTCAAAAAGCACAAGTGTCTGGACAAGGCGATAGTTTACATGAACATA
TTGCAAATTTAGCTGGTAGCCCTGCTATTAAAAAAGGTATTTTACAGACTGTAAAAGTTGTT
GATGAATTGGTCAAAGTAATGGGGCGGCATAAGCCAGAAAATATCGTTATTGAAATGGCACG
TGAAAATCAGACAACTCAAAAGGGCCAGAAAAATTCGCGAGAGCGTATGAAACGAATCGAAG
AAGGTATCAAAGAATTAGGAAGTCAGATTCTTAAAGAGCATCCTGTTGAAAATACTCAATTG
CAAAATGAAAAGCTCTATCTCTATTATCTCCAAAATGGAAGAGACATGTATGTGGACCAAGA
ATTAGATATTAATCGTTTAAGTGATTATGATGTCGATCACATTGTTCCACAAAGTTTCCTTA
AAGACGATTCAATAGACAATAAGGTCTTAACGCGTTCTGATAAAAATCGTGGTAAATCGGAT
AACGTTCCAAGTGAAGAAGTAGTCAAAAAGATGAAAAACTATTGGAGACAACTTCTAAACGC
CAAGTTAATCACTCAACGTAAGTTTGATAATTTAACGAAAGCTGAACGTGGAGGTTTGAGTG
AACTTGATAAAGCTGGTTTTATCAAACGCCAATTGGTTGAAACTCGCCAAATCACTAAGCAT
GTGGCACAAATTTTGGATAGTCGCATGAATACTAAATACGATGAAAATGATAAACTTATTCG
AGAGGTTAAAGTGATTACCTTAAAATCTAAATTAGTTTCTGACTTCCGAAAAGATTTCCAAT
TCTATAAAGTACGTGAGATTAACAATTACCATCATGCCCATGATGCGTATCTAAATGCCGTC
GTTGGAACTGCTTTGATTAAGAAATATCCAAAACTTGAATCGGAGTTTGTCTATGGTGATTA
TAAAGTTTATGATGTTCGTAAAATGATTGCTAAGTCTGAGCAAGAAATAGGCAAAGCAACCG
CAAAATATTTCTTTTACTCTAATATCATGAACTTCTTCAAAACAGAAATTACACTTGCAAAT
GGAGAGATTCGCAAACGCCCTCTAATCGAAACTAATGGGGAAACTGGAGAAATTGTCTGGGA
TAAAGGGCGAGATTTTGCCACAGTGCGCAAAGTATTGTCCATGCCCCAAGTCAATATTGTCA
AGAAAACAGAAGTACAGACAGGCGGATTCTCCAAGGAGTCAATTTTACCAAAAAGAAATTCG
GACAAGCTTATTGCTCGTAAAAAAGACTGGGATCCAAAAAAATATGGTGGTTTTGATAGTCC
AACGGTAGCTTATTCAGTCCTAGTGGTTGCTAAGGTGGAAAAAGGGAAATCGAAGAAGTTAA
AATCCGTTAAAGAGTTACTAGGGATCACAATTATGGAAAGAAGTTCCTTTGAAAAAAATCCG
ATTGACTTTTTAGAAGCTAAAGGATATAAGGAAGTTAAAAAAGACTTAATCATTAAACTACC
TAAATATAGTCTTTTTGAGTTAGAAAACGGTCGTAAACGGATGCTGGCTAGTGCCGGAGAAT
TACAAAAAGGAAATGAGCTGGCTCTGCCAAGCAAATATGTGAATTTTTTATATTTAGCTAGT
CATTATGAAAAGTTGAAGGGTAGTCCAGAAGATAACGAACAAAAACAATTGTTTGTGGAGCA
GCATAAGCATTATTTAGATGAGATTATTGAGCAAATCAGTGAATTTTCTAAGCGTGTTATTT
TAGCAGATGCCAATTTAGATAAAGTTCTTAGTGCATATAACAAACATAGAGACAAACCAATA
CGTGAACAAGCAGAAAATATTATTCATTTATTTACGTTGACGAATCTTGGAGCTCCCGCTGC
TTTTAAATATTTTGATACAACAATTGATCGTAAACGATATACGTCTACAAAAGAAGTTTTAG
ATGCCACTCTTATCCATCAATCCATCACTGGTCTTTATGAAACACGCATTGATTTGAGTCAG
CTAGGAGGTGACTGA
MDKKYSIGLDIGTNSVGWAVITDEYKVPSKKFKVLGNTDRHSIKKNLIGALLFDSGETAEAT
RLKRTARRRYTRRKNRICYLQEIFSNEMAKVDDSFFHRLEESFLVEEDKKHERHPIFGNIVD
EVAYHEKYPTIYHLRKKLVDSTDKADLRLIYLALAHMIKFRGHFLIEGDLNPDNSDVDKLFI
QLVQTYNQLFEENPINASGVDAKAILSARLSKSRRLENLIAQLPGEKKNGLFGNLIALSLGL
TPNFKSNFDLAEDAKLQLSKDTYDDDLDNLLAQIGDQYADLFLAAKNLSDAILLSDILRVNT
EITKAPLSASMIKRYDEHHQDLTLLKALVRQQLPEKYKEIFFDQSKNGYAGYIDGGASQEEF
YKFIKPILEKMDGTEELLVKLNREDLLRKQRTFDNGSIPHQIHLGELHAILRRQEDFYPFLK
DNREKIEKILTFRIPYYVGPLARGNSRFAWMTRKSEETITPWNFEEVVDKGASAQSFIERMT
NFDKNLPNEKVLPKHSLLYEYFTVYNELTKVKYVTEGMRKPAFLSGEQKKAIVDLLFKTNRK
VTVKQLKEDYFKKIECFDSVEISGVEDRFNASLGTYHDLLKIIKDKDFLDNEENEDILEDIV
LTLTLFEDREMIEERLKTYAHLFDDKVMKQLKRRRYTGWGRLSRKLINGIRDKQSGKTILDF
LKSDGFANRNFMQLIHDDSLTFKEDIQKAQVSGQGDSLHEHIANLAGSPAIKKGILQTVKVV
DELVKVMGRHKPENIVIEMARENQTTQKGQKNSRERMKRIEEGIKELGSQILKEHPVENTQL
QNEKLYLYYLQNGRDMYVDQELDINRLSDYDVDHIVPQSFLKDDSIDNKVLTRSDKNRGKSD
NVPSEEVVKKMKNYWRQLLNAKLITQRKFDNLTKAERGGLSELDKAGFIKRQLVETRQITKH
VAQILDSRMNTKYDENDKLIREVKVITLKSKLVSDFRKDFQFYKVREINNYHHAHDAYLNAV
VGTALIKKYPKLESEFVYGDYKVYDVRKMIAKSEQEIGKATAKYFFYSNIMNFFKTEITLAN
GEIRKRPLIETNGETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELALPSKYVNFLYLAS
HYEKLKGSPEDNEQKQLFVEQHKHYLDEIIEQISEFSKRVILADANLDKVLSAYNKHRDKPI
REQAENIIHLFTLTNLGAPAAFKYFDTTIDRKRYTSTKEVLDATLIHQSITGLYETRIDLSQ
LGGD
(single underline: HNH domain; double underline: RuvC domain)

In some embodiments, Cas9 refers to Cas9 from: Corynebacterium ulcerans (NCBI Refs: NC_015683.1, NC_017317.1); Corynebacterium diphtheria (NCBI Refs: NC_016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquis I (NCBI Ref: NC_018721.1); Streptococcus thermophilus (NCBI Ref: YP_820832.1), Listeria innocua (NCBI Ref: NP_472073.1), Campylobacter jejuni (NCBI Ref: YP_002344900.1) or Neisseria meningitidis (NCBI Ref: YP_002342100.1) or to a Cas9 from any other organism.

It should be appreciated that additional Cas9 proteins (e.g., a nuclease dead Cas9 (dCas9), a Cas9 nickase (nCas9), or a nuclease active Cas9), including variants and homologs thereof, are within the scope of this disclosure. Exemplary Cas9 proteins include, without limitation, those provided below. In some embodiments, the Cas9 protein is a nuclease dead Cas9 (dCas9). In some embodiments, the Cas9 protein is a Cas9 nickase (nCas9). In some embodiments, the Cas9 protein is a nuclease active Cas9.

In some embodiments, the Cas9 domain is a nuclease-inactive Cas9 domain (dCas9). For example, the dCas9 domain may bind to a duplexed nucleic acid molecule (e.g., via a gRNA molecule) without cleaving either strand of the duplexed nucleic acid molecule. In some embodiments, the nuclease-inactive dCas9 domain comprises a DIOX mutation and a H840X mutation of the amino acid sequence set forth herein, or a corresponding mutation in any of the amino acid sequences provided herein, wherein X is any amino acid change. In some embodiments, the nuclease-inactive dCas9 domain comprises a D10A mutation and a H840A mutation of the amino acid sequence set forth herein, or a corresponding mutation in any of the amino acid sequences provided herein. As one example, a nuclease-inactive Cas9 domain comprises the amino acid sequence set forth in Cloning vector pPlatTET-gRNA2 (Accession No. BAV54124).

The amino acid sequence of an exemplary catalytically inactive Cas9 (dCas9) is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

(see, e.g., Qi et al., “Repurposing CRISPR as an RNA-guided platform for sequence-specific control of gene expression.” Cell. 2013; 152(5):1173-83, the entire contents of which are incorporated herein by reference).

In some embodiments, a Cas9 nuclease has an inactive (e.g., an inactivated) DNA cleavage domain, that is, the Cas9 is a nickase, referred to as an “nCas9” protein (for “nickase” Cas9). A nuclease-inactivated Cas9 protein may interchangeably be referred to as a “dCas9” protein (for nuclease-“dead” Cas9) or catalytically inactive Cas9. Methods for generating a Cas9 protein (or a fragment thereof) having an inactive DNA cleavage domain are known (See, e.g., Jinek et al., Science. 337:816-821(2012); Qi et al., “Repurposing CRISPR as an RNA-Guided Platform for Sequence-Specific Control of Gene Expression” (2013) Cell. 28; 152(5):1173-83, the entire contents of each of which are incorporated herein by reference). For example, the DNA cleavage domain of Cas9 is known to include two subdomains, the HNH nuclease subdomain and the RuvC1 subdomain. The HNH subdomain cleaves the strand complementary to the gRNA, whereas the RuvC1 subdomain cleaves the non-complementary strand. Mutations within these subdomains can silence the nuclease activity of Cas9. For example, the mutations D10A and H840A completely inactivate the nuclease activity of S. pyogenes Cas9 (Jinek et al., Science. 337:816-821(2012); Qi et al., Cell. 28; 152(5):1173-83 (2013)).

In some embodiments, the dCas9 domain comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the dCas9 domains provided herein. In some embodiments, the Cas9 domain comprises an amino acid sequences that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more or more mutations compared to any one of the amino acid sequences set forth herein. In some embodiments, the Cas9 domain comprises an amino acid sequence that has at least 10, at least 15, at least 20, at least 30, at least 40, at least 50, at least 60, at least 70, at least 80, at least 90, at least 100, at least 150, at least 200, at least 250, at least 300, at least 350, at least 400, at least 500, at least 600, at least 700, at least 800, at least 900, at least 1000, at least 1100, or at least 1200 identical contiguous amino acid residues as compared to any one of the amino acid sequences set forth herein.

In some embodiments, dCas9 corresponds to, or comprises in part or in whole, a Cas9 amino acid sequence having one or more mutations that inactivate the Cas9 nuclease activity. For example, in some embodiments, a dCas9 domain comprises D10A and an H840A mutation or corresponding mutations in another Cas9.

In some embodiments, the dCas9 comprises the amino acid sequence of dCas9 (D10A and H840A):

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDAIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain; double underline:
RuvC domain).

In some embodiments, the Cas9 domain comprises a D10A mutation, while the residue at position 840 remains a histidine in the amino acid sequence provided above, or at corresponding positions in any of the amino acid sequences provided herein.

In other embodiments, dCas9 variants having mutations other than D10A and H840A are provided, which, e.g., result in nuclease inactivated Cas9 (dCas9). Such mutations, by way of example, include other amino acid substitutions at D10 and H840, or other substitutions within the nuclease domains of Cas9 (e.g., substitutions in the HNH nuclease subdomain and/or the RuvC1 subdomain). In some embodiments, variants or homologues of dCas9 are provided which are at least about 70% identical, at least about 80% identical, at least about 90% identical, at least about 95% identical, at least about 98% identical, at least about 99% identical, at least about 99.5% identical, or at least about 99.9% identical. In some embodiments, variants of dCas9 are provided having amino acid sequences which are shorter, or longer, by about 5 amino acids, by about 10 amino acids, by about 15 amino acids, by about 20 amino acids, by about 25 amino acids, by about 30 amino acids, by about 40 amino acids, by about 50 amino acids, by about 75 amino acids, by about 100 amino acids or more.

In some embodiments, the Cas9 domain is a Cas9 nickase. The Cas9 nickase may be a Cas9 protein that is capable of cleaving only one strand of a duplexed nucleic acid molecule (e.g., a duplexed DNA molecule). In some embodiments, the Cas9 nickase cleaves the target strand of a duplexed nucleic acid molecule, meaning that the Cas9 nickase cleaves the strand that is base paired to (complementary to) a gRNA (e.g., an sgRNA) that is bound to the Cas9. In some embodiments, a Cas9 nickase comprises a D10A mutation and has a histidine at position 840. In some embodiments, the Cas9 nickase cleaves the non-target, non-base-edited strand of a duplexed nucleic acid molecule, meaning that the Cas9 nickase cleaves the strand that is not base paired to a gRNA (e.g., an sgRNA) that is bound to the Cas9. In some embodiments, a Cas9 nickase comprises an H840A mutation and has an aspartic acid residue at position 10, or a corresponding mutation. In some embodiments, the Cas9 nickase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the Cas9 nickases provided herein. Additional suitable Cas9 nickases will be apparent to those of skill in the art based on this disclosure and knowledge in the field, and are within the scope of this disclosure.

The amino acid sequence of an exemplary catalytically Cas9 nickase (nCas9) is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD

In some embodiments, Cas9 refers to a Cas9 from archaea (e.g., nanoarchaea), which constitute a domain and kingdom of single-celled prokaryotic microbes. In some embodiments, the programmable nucleotide binding protein may be a CasX or CasY protein, which have been described in, for example, Burstein et al., “New CRISPR-Cas systems from uncultivated microbes.” Cell Res. 2017 Feb. 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little-studied nanoarchaea as part of an active CRISPR-Cas system. In bacteria, two previously unknown systems were discovered, CRISPR-CasX and CRISPR-CasY, which are among the most compact systems yet discovered. In some embodiments, in a base editor system described herein Cas9 is replaced by CasX, or a variant of CasX. In some embodiments, in a base editor system described herein Cas9 is replaced by CasY, or a variant of CasY. It should be appreciated that other RNA-guided DNA binding proteins may be used as a nucleic acid programmable DNA binding protein (napDNAbp), and are within the scope of this disclosure.

In some embodiments, the nucleic acid programmable DNA binding protein (napDNAbp) of any of the fusion proteins provided herein may be a CasX or CasY protein. In some embodiments, the napDNAbp is a CasX protein. In some embodiments, the napDNAbp is a CasY protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring CasX or CasY protein. In some embodiments, the programmable nucleotide binding protein is a naturally-occurring CasX or CasY protein. In some embodiments, the programmable nucleotide binding protein comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any CasX or CasY protein described herein. It should be appreciated that CasX and CasY from other bacterial species may also be used in accordance with the present disclosure.

An exemplary CasX ((uniprot.org/uniprot/F0NN87; uniprot.org/uniprot/F0NH53) tr|F0NN87|F0NN87_SULIHCRISPR-associatedCasx protein OS=Sulfolobus islandicus (strain HVE10/4) GN=SiH_0402 PE=4 SV=1) amino acid sequence is as follows:

MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYEFGRSPGMVERTRRVKLE
VEPHYLIIAAAGWVLTRLGKAKVSEGDYVGVNVFTPTRGILYSLIQNVNG
IVPGIKPETAFGLWIARKVVSSVTNPNVSVVRIYTISDAVGQNPTTINGG
FSIDLTKLLEKRYLLSERLEAIARNALSISSNMRERYIVLANYIYEYLTG
SKRLEDLLYFANRDLIMNLNSDDGKVRDLKLISAYVNGELIRGEG.

An exemplary CasX (>tr|F0NH53|F0NH53_SULIR CRISPR associated protein, Casx OS=Sulfolobus islandicus (strain REY15A) GN=SiRe_0771 PE=4 SV=1) amino acid sequence is as follows:

MEVPLYNIFGDNYIIQVATEAENSTIYNNKVEIDDEELRNVLNLAYKIAK
NNEDAAAERRGKAKKKKGEEGETTTSNIILPLSGNDKNPWTETLKCYNFP
TTVALSEVFKNFSQVKECEEVSAPSFVKPEFYKFGRSPGMVERTRRVKLE
VEPHYLIMAAAGWVLTRLGKAKVSEGDYVGVNVFTPTRGILYSLIQNVNG
IVPGIKPETAFGLWIARKVVSSVTNPNVSVVSIYTISDAVGQNPTTINGG
FSIDLTKLLEKRDLLSERLEAIARNALSISSNMRERYIVLANYIYEYLTG
SKRLEDLLYFANRDLIMNLNSDDGKVRDLKLISAYVNGELIRGEG.
Deltaproteobacteria CasX
MEKRINKIRKKLSADNATKPVSRSGPMKTLLVRVMTDDLKKRLEKRRKKP
EVMPQVISNNAANNLRMLLDDYTKMKEAILQVYWQEFKDDHVGLMCKFAQ
PASKKIDQNKLKPEMDEKGNLTTAGFACSQCGQPLFVYKLEQVSEKGKAY
TNYFGRCNVAEHEKLILLAQLKPVKDSDEAVTYSLGKFGQRALDFYSIHV
TKESTHPVKPLAQIAGNRYASGPVGKALSDACMGTIASFLSKYQDIIIEH
QKVVKGNQKRLESLRELAGKENLEYPSVTLPPQPHTKEGVDfAYNEVIAR
VRMWVNLNLWQKLKLSRDDAKPLLRLKGFPSFPVVERRENEVDWWNTINE
VKKLIDAKRDMGRVFWSGVTAEKRNTILEGYNYLPNENDHKKREGSLENP
KKPAKRQFGDLLLYLEKKYAGDWGKVFDEAWERIDKKIAGLTSHIEREEA
RNAEDAQSKAVLTDWLRAKASFVLERLKEMDEKEFYACEIQLQKWYGDLR
GNPFAVEAENRVVDISGFSIGSDGHSIQYRNLLAWKYLENGKREFYLLMN
YGKKGRIRFTDGTDIKKSGKWQGLLYGGGKAKVIDLTFDPDDEQLIILPL
AFGTRQGREFIWNDLLSLETGLIKLANGRVIEKTIYNKKIGRDEPALFVA
LTFERREVVDPSNIKPVNLIGVARGENIPAVIALTDPEGCPLPEFKDSSG
GPTDILRIGEGYKEKQRAIQAAKEVEQRRAGGYSRKFASKSRNLADDMVR
NSARDLFYHAVTHDAVLVFANLSRGFGRQGKRTFMTERQYTKMEDWLTAK
LAYEGLTSKTYLSKTLAQYTSKTCSNCGFTITYADMDVMLVRLKKTSDGW
ATTLNNKELKAEYQITYYNRYKRQTVEKELSAELDRLSEESGNNDISKWT
KGRRDEALFLLKKRFSHRPVQEQFVCLDCGHEVHAAEQAALNIARSWLFL
NSNSTEFKSYKSGKQPFVGAWQAFYKRRLKEVWKPNA

An exemplary CasY ((ncbi.nlm.nih.gov/protein/APG80656.1)>APG80656.1 CRISPR-associated protein CasY [uncultured Parcubacteria group bacterium]) amino acid sequence is as follows:

MSKRHPRISGVKGYRLHAQRLEYTGKSGAMRTIKYPLYSSPSGGRTVPR
EIVSAINDDYVGLYGLSNFDDLYNAEKRNEEKVYSVLDFWYDCVQYGAV
FSYTAPGLLKNVAEVRGGSYELTKTLKGSHLYDELQIDKVIKFLNKKEI
SRANGSLDKLKKDIIDCFKAEYRERHKDQCNKLADDIKNAKKDAGASLG
ERQKKLFRDFFGISEQSENDKPSFTNPLNLTCCLLPFDTVNNNRNRGEV
LFNKLKEYAQKLDKNEGSLEMWEYIGIGNSGTAFSNFLGEGFLGRLREN
KITELKKAMMDITDAWRGQEQEEELEKRLRILAALTIKLREPKFDNHWG
GYRSDINGKLSSWLQNYINQTVKIKEDLKGHKKDLKKAKEMINRFGESD
TKEEAVVSSLLESIEKIVPDDSADDEKPDIPAIAIYRRFLSDGRLTLNR
FVQREDVQEALIKERLEAEKKKKPKKRKKKSDAEDEKETIDFKELFPHL
AKPLKLVPNFYGDSKRELYKKYKNAAIYTDALWKAVEKIYKSAFSSSLK
NSFFDTDFDKDFFIKRLQKIFSVYRRFNTDKWKPIVKNSFAPYCDIVSL
AENEVLYKPKQSRSRKSAAIDKNRVRLPSTENIAKAGIALARELSVAGF
DWKDLLKKEEHEEYIDLIELHKTALALLLAVTETQLDISALDFVENGTV
KDFMKTRDGNLVLEGRFLEMFSQSIVFSELRGLAGLMSRKEFITRSAIQ
TMNGKQAELLYIPHEFQSAKITTPKEMSRAFLDLAPAEFATSLEPESLS
EKSLLKLKQMRYYPHYFGYELTRTGQGIDGGVAENALRLEKSPVKKREI
KCKQYKTLGRGQNKIVLYVRSSYYQTQFLEWFLHRPKNVQTDVAVSGSF
LIDEKKVKTRWNYDALTVALEPVSGSERVFVSQPFTIFPEKSAEEEGQR
YLGIDIGEYGIAYTALEITGDSAKILDQNFISDPQLKTLREEVKGLKLD
QRRGTFAMPSTKIARIRESLVHSLRNRIHHLALKHKAKIVYELEVSRFE
EGKQKIKKVYATLKKADVYSEIDADKNLQTTVWGKLAVASEISASYTSQ
FCGACKKLWRAEMQVDETITTQELIGTVRVIKGGTLIDAIKDFMRPPIF
DENDTPFPKYRDFCDKHHISKKMRGNSCLFICPFCRANADADIQASQTI
ALLRYVKEEKKVEDYFERFRKLKNIKVLGQMKKI.

In some embodiments, the nucleic acid programmable DNA binding protein (napDNAbp) is a single effector of a microbial CRISPR-Cas system. Single effectors of microbial CRISPR-Cas systems include, without limitation, Cas9, Cpf1, Cas12b/C2c1, and Cas12c/C2c3. Typically, microbial CRISPR-Cas systems are divided into Class 1 and Class 2 systems. Class 1 systems have multisubunit effector complexes, while Class 2 systems have a single protein effector. For example, Cas9 and Cpf1 are Class 2 effectors. In addition to Cas9 and Cpf1, three distinct Class 2 CRISPR-Cas systems (Cas12b/C2c1, and Cas12c/C2c3) have been described by Shmakov et al., “Discovery and Functional Characterization of Diverse Class 2 CRISPR Cas Systems”, Mol. Cell, 2015 Nov. 5; 60(3): 385-397, the entire contents of which is hereby incorporated by reference. Effectors of two of the systems, Cas12b/C2c1, and Cas12c/C2c3, contain RuvC-like endonuclease domains related to Cpf1. A third system contains an effector with two predicated HEPN RNase domains. Production of mature CRISPR RNA is tracrRNA-independent, unlike production of CRISPR RNA by Cas12b/C2c1. Cas12b/C2c1 depends on both CRISPR RNA and tracrRNA for DNA cleavage.

The crystal structure of Alicyclobaccillus acidoterrastris Cas12b/C2c1 (AacC2c1) has been reported in complex with a chimeric single-molecule guide RNA (sgRNA). See e.g., Liu et al., “C2c1-sgRNA Complex Structure Reveals RNA-Guided DNA Cleavage Mechanism”, Mol. Cell, 2017 Jan. 19; 65(2):310-322, the entire contents of which are hereby incorporated by reference. The crystal structure has also been reported in Alicyclobacillus acidoterrestris C2c1 bound to target DNAs as ternary complexes. See e.g., Yang et al., “PAM-dependent Target DNA Recognition and Cleavage by C2C1 CRISPR-Cas endonuclease”, Cell, 2016 Dec. 15; 167(7):1814-1828, the entire contents of which are hereby incorporated by reference. Catalytically competent conformations of AacC2c1, both with target and non-target DNA strands, have been captured independently positioned within a single RuvC catalytic pocket, with Cas12b/C2c1-mediated cleavage resulting in a staggered seven-nucleotide break of target DNA. Structural comparisons between Cas12b/C2c1 ternary complexes and previously identified Cas9 and Cpf1 counterparts demonstrate the diversity of mechanisms used by CRISPR-Cas9 systems.

In some embodiments, the nucleic acid programmable DNA binding protein (napDNAbp) of any of the fusion proteins provided herein may be a Cas12b/C2c1, or a Cas12c/C2c3 protein. In some embodiments, the napDNAbp is a Cas12b/C2c1 protein. In some embodiments, the napDNAbp is a Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a naturally-occurring Cas12b/C2c1 or Cas12c/C2c3 protein. In some embodiments, the napDNAbp is a naturally-occurring Cas12b/C2c1 or Cas12c/C2c3 protein. In some embodiments, the napDNAbp comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to any one of the napDNAbp sequences provided herein. It should be appreciated that Cas12b/C2c1 or Cas12c/C2c3 from other bacterial species may also be used in accordance with the present disclosure.

A Cas12b/C2c1 ((uniprot.org/uniprot/T0D7A2#2) sp|T0D7A2|C2C1_ALIAG CRISPR-associated endonuclease C2c1 OS=Alicyclobacillus acido-terrestris (strain ATCC 49025/DSM 3922/CIP 106132/NCIMB 13137/GD3B) GN=c2c1 PE=1 SV=1) amino acid sequence is as follows:

MAVKSIKVKLRLDDMPEIRAGLWKLHKEVNAGVRYYTEWLSLLRQENLYR
RSPNGDGEQECDKTAEECKAELLERLRARQVENGHRGPAGSDDELLQLAR
QLYELLVPQAIGAKGDAQQIARKFLSPLADKDAVGGLGIAKAGNKPRWVR
MREAGEPGWEEEKEKAETRKSADRTADVLRALADFGLKPLMRVYTDSEMS
SVEWKPLRKGQAVRTWDRDMFQQAIERMMSWESWNQRVGQEYAKLVEQKN
RFEQKNFVGQEHLVHLVNQLQQDMKEASPGLESKEQTAHYVTGRALRGSD
KVFEKWGKLAPDAPFDLYDAEIKNVQRRNTRRFGSHDLFAKLAEPEYQAL
WREDASFLTRYAVYNSILRKLNHAKMFATFTLPDATAHPIWTRFDKLGGN
LHQYTFLFNEFGERRHAIRFHKLLKVENGVAREVDDVTVPISMSEQLDNL
LPRDPNEPIALYFRDYGAEQHFTGEFGGAKIQCRRDQLAHMHRRRGARDV
YLNVSVRVQSQSEARGERRPPYAAVFRLVGDNHRAFVHFDKLSDYLAEHP
DDGKLGSEGLLSGLRVMSVDLGLRTSASISVFRVARKDELKPNSKGRVPF
FFPIKGNDNLVAVHERSQLLKLPGETESKDLRAIREERQRTLRQLRTQLA
YLRLLVRCGSEDVGRRERSWAKLIEQPVDAANHMTPDWREAFENELQKLK
SLHGICSDKEWMDAVYESVRRVWRHMGKQVRDWRKDVRSGERPKIRGYAK
DVVGGNSIEQIEYLERQYKFLKSWSFFGKVSGQVIRAEKGSRFAITLREH
IDHAKEDRLKKLADRIIMEALGYVYALDERGKGKWVAKYPPCQLILLEEL
SEYQFNNDRPPSENNQLMQWSHRGVFQELINQAQVHDLLVGTMYAAFSSR
FDARTGAPGIRCRRVPARCTQEHNPEPFPWWLNKFVVEHTLDACPLRADD
LIPTGEGEIFVSPFSAEEGDFHQIHADLNAAQNLQQRLWSDFDISQIRLR
CDWGEVDGELVLIPRLTGKRTADSYSNKVFYTNTGVTYYERERGKKRRKV
FAQEKLSEEEAELLVEADEAREKSVVLMRDPSGIINRGNWTRQKEFWSMV
NQRIEGYLVKQIRSRVPLQDSACENTGDI

BhCas12b (Bacillus hisashii) NCBI Reference Sequence: WP 095142515

MAPKKKRKVGIHGVPAAATRSFILKIEPNEEVKKGLWKTHEVLNHGIAYY
MNILKLIRQEAIYEHHEQDPKNPKKVSKAEIQAELWDFVLKMQKCNSFTH
EVDKDEVFNILRELYEELVPSSVEKKGEANQLSNKFLYPLVDPNSQSGKG
TASSGRKPRWYNLKIAGDPSWEEEKKKWEEDKKKDPLAKILGKLAEYGLI
PLFIPYTDSNEPIVKEIKWMEKSRNQSVRRLDKDMFIQALERFLSWESWN
LKVKEEYEKVEKEYKTLEERIKEDIQALKALEQYEKERQEQLLRDTLNTN
EYRLSKRGLRGWREIIQKWLKMDENEPSEKYLEVFKDYQRKHPREAGDYS
VYEFLSKKENHFIWRNHPEYPYLYATFCEIDKKKKDAKQQATFTLADPIN
HPLWVRFEERSGSNLNKYRILTEQLHTEKLKKKLTVQLDRLIYPTESGGW
EEKGKVDIVLLPSRQFYNQIFLDIEEKGKHAFTYKDESIKFPLKGTLGGA
RVQFDRDHLRRYPHKVESGNVGRIYFNMTVNIEPTESPVSKSLKIHRDDF
PKVVNFKPKELTEWIKDSKGKKLKSGIESLEIGLRVMSIDLGQRQAAAAS
IFEVVDQKPDIEGKLFFPIKGTELYAVHRASFNIKLPGETLVKSREVLRK
AREDNLKLMNQKLNFLRNVLHFQQFEDITEREKRVTKWISRQENSDVPLV
YQDELIQIRELMYKPYKDWVAFLKQLHKRLEVEIGKEVKHWRKSLSDGRK
GLYGISLKNIDEIDRTRKFLLRWSLRPTEPGEVRRLEPGQRFAIDQLNHL
NALKEDRLKKMANTIIMHALGYCYDVRKKKWQAKNPACQIILFEDLSNYN
PYEERSRFENSKLMKWSRREIPRQVALQGEIYGLQVGEVGAQFSSRFHAK
TGSPGIRCSVVTKEKLQDNRFFKNLQREGRLTLDKIAVLKEGDLYPDKGG
EKFISLSKDRKCVTTHADINAAQNLQKRFWTRTHGFYKVYCKAYQVDGQT
VYIPESKDQKQKIIEEFGEGYFILKDGVYEWVNAGKLKIKKGSSKQSSSE
LVDSDILKDSFDLASELKGEKLMLYRDPSGNVFPSDKWMAAGVFFGKLER
ILISKLTNQYSISTIEDDSSKQSMKRPAATKKAGQAKKKK

In some embodiments, the Cas12b is BvCas12B, which is a variant of BhCas12b and comprises the following changes relative to BhCas12B: S893R, K846R, and E837G. BvCas12b (Bacillus sp. V3-13) NCBI Reference Sequence: WP_101661451.1

MAIRSIKLKMKTNSGTDSIYLRKALWRTHQLINEGIAYYMNLLTLYRQEA
IGDKTKEAYQAELINIIRNQQRNNGSSEEHGSDQEILALLRQLYELIIPS
SIGESGDANQLGNKFLYPLVDPNSQSGKGTSNAGRKPRWKRLKEEGNPDW
ELEKKKDEERKAKDPTVKIFDNLNKYGLLPLFPLFTNIQKDIEWLPLGKR
QSVRKWDKDMFIQAIERLLSWESWNRRVADEYKQLKEKTESYYKEHLTGG
EEWIEKIRKFEKERNMELEKNAFAPNDGYFITSRQIRGWDRVYEKWSKLP
ESASPEELWKVVAEQQNKMSEGFGDPKVFSFLANRENRDIWRGHSERIYH
IAAYNGLQKKLSRTKEQATFTLPDAIEHPLWIRYESPGGTNLNLFKLEEK
QKKNYYVTLSKIIWPSEEKWIEKENIEIPLAPSIQFNRQIKLKQHVKGKQ
EISFSDYSSRISLDGVLGGSRIQFNRKYIKNHKELLGEGDIGPVFFNLVV
DVAPLQETRNGRLQSPIGKALKVISSDFSKVIDYKPKELMDWMNTGSASN
SFGVASLLEGMRVMSIDMGQRTSASVSIFEVVKELPKDQEQKLFYSINDT
ELFAIHKRSFLLNLPGEVVTKNNKQQRQERRKKRQFVRSQIRMLANVLRL
ETKKTPDERKKAIHKLMEIVQSYDSWTASQKEVWEKELNLLTNMAAFNDE
IWKESLVELHHRIEPYVGQIVSKWRKGLSEGRKNLAGISMWNIDELEDTR
RLLISWSKRSRTPGEANRIETDEPFGSSLLQHIQNVKDDRLKQMANLIIM
TALGFKYDKEEKDRYKRWKETYPACQIILFENLNRYLFNLDRSRRENSRL
MKWAHRSIPRTVSMQGEMFGLQVGDVRSEYSSRFHAKTGAPGIRCHALTE
EDLKAGSNTLKRLIEDGFINESELAYLKKGDIIPSQGGELFVTLSKRYKK
DSDNNELTVIHADINAAQNLQKRFWQQNSEVYRVPCQLARMGEDKLYIPK
SQTETIKKYFGKGSFVKNNTEQEVYKWEKSEKMKIKTDTTFDLQDLDGFE
DISKTIELAQEQQKKYLTMFRDPSGYFFNNETWRPQKEYWSIVNNIIKSC
LKKKILSNKVEL

The Cas9 nuclease has two functional endonuclease domains: RuvC and HNH. Cas9 undergoes a conformational change upon target binding that positions the nuclease domains to cleave opposite strands of the target DNA. The end result of Cas9-mediated DNA cleavage is a double-strand break (DSB) within the target DNA (˜3-4 nucleotides upstream of the PAM sequence). The resulting DSB is then repaired by one of two general repair pathways: (1) the efficient but error-prone non-homologous end joining (NHEJ) pathway; or (2) the less efficient but high-fidelity homology directed repair (HDR) pathway.

The “efficiency” of non-homologous end joining (NHEJ) and/or homology directed repair (HDR) can be calculated by any convenient method. For example, in some cases, efficiency can be expressed in terms of percentage of successful HDR. For example, a surveyor nuclease assay can be used to generate cleavage products and the ratio of products to substrate can be used to calculate the percentage. For example, a surveyor nuclease enzyme can be used that directly cleaves DNA containing a newly integrated restriction sequence as the result of successful HDR. More cleaved substrate indicates a greater percent HDR (a greater efficiency of HDR). As an illustrative example, a fraction (percentage) of HDR can be calculated using the following equation [(cleavage products)/(substrate plus cleavage products)] (e.g., (b+c)/(a+b+c), where “a” is the band intensity of DNA substrate and “b” and “c” are the cleavage products).

In some cases, efficiency can be expressed in terms of percentage of successful NHEJ. For example, a T7 endonuclease I assay can be used to generate cleavage products and the ratio of products to substrate can be used to calculate the percentage NHEJ. T7 endonuclease I cleaves mismatched heteroduplex DNA which arises from hybridization of wild-type and mutant DNA strands (NHEJ generates small random insertions or deletions (indels) at the site of the original break). More cleavage indicates a greater percent NHEJ (a greater efficiency of NHEJ). As an illustrative example, a fraction (percentage) of NHEJ can be calculated using the following equation: (1−(1−(b+c)/(a+b+c))^{1/2})×100, where “a” is the band intensity of DNA substrate and “b” and “c” are the cleavage products (Ran et. al., Cell. 2013 Sep. 12; 154(6):1380-9; and Ran et al., Nat Protoc. 2013 November; 8(11): 2281-2308).

The NHEJ repair pathway is the most active repair mechanism, and it frequently causes small nucleotide insertions or deletions (indels) at the DSB site. The randomness of NHEJ-mediated DSB repair has important practical implications, because a population of cells expressing Cas9 and a gRNA or a guide polynucleotide can result in a diverse array of mutations. In most cases, NHEJ gives rise to small indels in the target DNA that result in amino acid deletions, insertions, or frameshift mutations leading to premature stop codons within the open reading frame (ORF) of the targeted gene. The ideal end result is a loss-of-function mutation within the targeted gene.

While NHEJ-mediated DSB repair often disrupts the open reading frame of the gene, homology directed repair (HDR) can be used to generate specific nucleotide changes ranging from a single nucleotide change to large insertions like the addition of a fluorophore or tag. In order to utilize HDR for gene editing, a DNA repair template containing the desired sequence can be delivered into the cell type of interest with the gRNA(s) and Cas9 or Cas9 nickase. The repair template can contain the desired edit as well as additional homologous sequence immediately upstream and downstream of the target (termed left & right homology arms). The length of each homology arm can be dependent on the size of the change being introduced, with larger insertions requiring longer homology arms. The repair template can be a single-stranded oligonucleotide, double-stranded oligonucleotide, or a double-stranded DNA plasmid. The efficiency of HDR is generally low (<10% of modified alleles) even in cells that express Cas9, gRNA and an exogenous repair template. The efficiency of HDR can be enhanced by synchronizing the cells, since HDR takes place during the S and G2 phases of the cell cycle. Chemically or genetically inhibiting genes involved in NHEJ can also increase HDR frequency.

In some embodiments, Cas9 is a modified Cas9. A given gRNA targeting sequence can have additional sites throughout the genome where partial homology exists. These sites are called off-targets and need to be considered when designing a gRNA. In addition to optimizing gRNA design, CRISPR specificity can also be increased through modifications to Cas9. Cas9 generates double-strand breaks (DSBs) through the combined activity of two nuclease domains, RuvC and HNH. Cas9 nickase, a D10A mutant of SpCas9, retains one nuclease domain and generates a DNA nick rather than a DSB. The nickase system can also be combined with HDR-mediated gene editing for specific gene edits.

In some cases, Cas9 is a variant Cas9 protein. A variant Cas9 polypeptide has an amino acid sequence that is different by one amino acid (e.g., has a deletion, insertion, substitution, fusion) when compared to the amino acid sequence of a wild type Cas9 protein. In some instances, the variant Cas9 polypeptide has an amino acid change (e.g., deletion, insertion, or substitution) that reduces the nuclease activity of the Cas9 polypeptide. For example, in some instances, the variant Cas9 polypeptide has less than 50%, less than 40%, less than 30%, less than 20%, less than 10%, less than 5%, or less than 1% of the nuclease activity of the corresponding wild-type Cas9 protein. In some cases, the variant Cas9 protein has no substantial nuclease activity. When a subject Cas9 protein is a variant Cas9 protein that has no substantial nuclease activity, it can be referred to as “dCas9.”

In some cases, a variant Cas9 protein has reduced nuclease activity. For example, a variant Cas9 protein exhibits less than about 20%, less than about 15%, less than about 10%, less than about 5%, less than about 1%, or less than about 0.1%, of the endonuclease activity of a wild-type Cas9 protein, e.g., a wild-type Cas9 protein.

In some cases, a variant Cas9 protein can cleave the complementary strand of a guide target sequence but has reduced ability to cleave the non-complementary strand of a double stranded guide target sequence. For example, the variant Cas9 protein can have a mutation (amino acid substitution) that reduces the function of the RuvC domain. As a non-limiting example, in some embodiments, a variant Cas9 protein has a D10A (aspartate to alanine at amino acid position 10) and can therefore cleave the complementary strand of a double stranded guide target sequence but has reduced ability to cleave the non-complementary strand of a double stranded guide target sequence (thus resulting in a single strand break (SSB) instead of a double strand break (DSB) when the variant Cas9 protein cleaves a double stranded target nucleic acid) (see, for example, Jinek et al., Science. 2012 Aug. 17; 337(6096):816-21).

In some cases, a variant Cas9 protein can cleave the non-complementary strand of a double stranded guide target sequence but has reduced ability to cleave the complementary strand of the guide target sequence. For example, the variant Cas9 protein can have a mutation (amino acid substitution) that reduces the function of the HNH domain (RuvC/HNH/RuvC domain motifs). As a non-limiting example, in some embodiments, the variant Cas9 protein has an H840A (histidine to alanine at amino acid position 840) mutation and can therefore cleave the non-complementary strand of the guide target sequence but has reduced ability to cleave the complementary strand of the guide target sequence (thus resulting in a SSB instead of a DSB when the variant Cas9 protein cleaves a double stranded guide target sequence). Such a Cas9 protein has a reduced ability to cleave a guide target sequence (e.g., a single stranded guide target sequence) but retains the ability to bind a guide target sequence (e.g., a single stranded guide target sequence).

In some cases, a variant Cas9 protein has a reduced ability to cleave both the complementary and the non-complementary strands of a double stranded target DNA. As a non-limiting example, in some cases, the variant Cas9 protein harbors both the D10A and the H840A mutations such that the polypeptide has a reduced ability to cleave both the complementary and the non-complementary strands of a double stranded target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors W476A and W1126A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA).

As another non-limiting example, in some cases, the variant Cas9 protein harbors H840A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors H840A, D10A, W476A, and W1126A, mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some embodiments, the variant Cas9 has restored catalytic His residue at position 840 in the Cas9 HNH domain (A840H).

As another non-limiting example, in some cases, the variant Cas9 protein harbors, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors D10A, H840A, P475A, W476A, N477A, D1125A, W1126A, and D1127A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some cases, when a variant Cas9 protein harbors W476A and W1126A mutations or when the variant Cas9 protein harbors P475A, W476A, N477A, D1125A, W1126A, and D1127A mutations, the variant Cas9 protein does not bind efficiently to a PAM sequence. Thus, in some such cases, when such a variant Cas9 protein is used in a method of binding, the method does not require a PAM sequence. In other words, in some cases, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a variant Cas9 protein that has reduced catalytic activity (e.g., when a Cas9 protein has a D10, G12, G17, E762, H840, N854, N863, H982, H983, A984, D986, and/or a A987 mutation, e.g., D10A, G12A, G17A, E762A, H840A, N854A, N863A, H982A, H983A, A984A, and/or D986A), the variant Cas9 protein can still bind to target DNA in a site-specific manner (because it is still guided to a target DNA sequence by a guide RNA) as long as it retains the ability to interact with the guide RNA.

In some embodiments, the variant Cas protein can be spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, a modified SpCas9 including amino acid substitutions D1135M, S1136Q, G1218K, E1219F, A1322R, D1332A, R1335E, and T1337R (SpCas9-MQKFRAER) and having specificity for the altered PAM 5′-NGC-3′ was used.

Alternatives to S. pyogenes Cas9 can include RNA-guided endonucleases from the Cpf1 family that display cleavage activity in mammalian cells. CRISPR from Prevotella and Francisella I (CRISPR/Cpf1) is a DNA-editing technology analogous to the CRISPR/Cas9 system. Cpf1 is an RNA-guided endonuclease of a class II CRISPR/Cas system. This acquired immune mechanism is found in Prevotella and Francisella bacteria. Cpf1 genes are associated with the CRISPR locus, coding for an endonuclease that use a guide RNA to find and cleave viral DNA. Cpf1 is a smaller and simpler endonuclease than Cas9, overcoming some of the CRISPR/Cas9 system limitations. Unlike Cas9 nucleases, the result of Cpf1-mediated DNA cleavage is a double-strand break with a short 3′ overhang. Cpf1's staggered III cleavage pattern can open up the possibility of directional gene transfer, analogous to traditional restriction enzyme cloning, which can increase the efficiency of gene editing. Like the Cas9 variants and orthologues described above, Cpf1 can also expand the number of sites that can be targeted by CRISPR to AT-rich regions or AT-rich genomes that lack the NGG PAM sites favored by SpCas9. The Cpf1 locus contains a mixed alpha/beta domain, a RuvC-I followed by a helical region, a RuvC-II and a zinc finger-like domain. The Cpf1 protein has a RuvC-like endonuclease domain that is similar to the RuvC domain of Cas9. Furthermore, Cpf1 does not have a HNH endonuclease domain, and the N-terminal of Cpf1 does not have the alpha-helical recognition lobe of Cas9. Cpf1 CRISPR-Cas domain architecture shows that Cpf1 is functionally unique, being classified as Class 2, type V CRISPR system. The Cpf1 loci encode Cas1, Cas2 and Cas4 proteins more similar to types I and III than from type II systems. Functional Cpf1 doesn't need the trans-activating CRISPR RNA (tracrRNA), therefore, only CRISPR (crRNA) is required. This benefits genome editing because Cpf1 is not only smaller than Cas9, but also it has a smaller sgRNA molecule (proximately half as many nucleotides as Cas9). The Cpf1-crRNA complex cleaves target DNA or RNA by identification of a protospacer adjacent motif 5′-YTN-3′ in contrast to the G-rich PAM targeted by Cas9. After identification of PAM, Cpf1 introduces a sticky-end-like DNA double-stranded break of 4 or 5 nucleotides overhang.

Some aspects of the disclosure provide fusion proteins comprising domains that act as nucleic acid programmable DNA binding proteins, which may be used to guide a protein, such as a base editor, to a specific nucleic acid (e.g., DNA or RNA) sequence. In particular embodiments, a fusion protein comprises a nucleic acid programmable DNA binding protein domain and a deaminase domain. DNA binding proteins include, without limitation, Cas9 (e.g., dCas9 and nCas9), Cas12a/Cpf1, Cas12b/C2c1, Cas12c/C2c3, Cas12d/CasY, Cas12e/CasX, Cas12g, Cas12h, and Cas12i. One example of a programmable polynucleotide-binding protein that has different PAM specificity than Cas9 is Clustered Regularly Interspaced Short Palindromic Repeats from Prevotella and Francisella 1 (Cpf1). Similar to Cas9, Cpf1 is also a class 2 CRISPR effector. It has been shown that Cpf1 mediates robust DNA interference with features distinct from Cas9. Cpf1 is a single RNA-guided endonuclease lacking tracrRNA, and it utilizes a T-rich protospacer-adjacent motif (TTN, TTTN, or YTN). Moreover, Cpf1 cleaves DNA via a staggered DNA double-stranded break. Out of 16 Cpf1-family proteins, two enzymes from Acidaminococcus and Lachnospiraceae are shown to have efficient genome-editing activity in human cells. Cpf1 proteins are known in the art and have been described previously, for example Yamano et al., “Crystal structure of Cpf1 in complex with guide RNA and target DNA.” Cell (165) 2016, p. 949-962; the entire contents of which is hereby incorporated by reference.

Also useful in the present compositions and methods are nuclease-inactive Cpf1 (dCpf1) variants that may be used as a guide nucleotide sequence-programmable polynucleotide-binding protein domain. The Cpf1 protein has a RuvC-like endonuclease domain that is similar to the RuvC domain of Cas9 but does not have a HNH endonuclease domain, and the N-terminal of Cpf1 does not have the alfa-helical recognition lobe of Cas9. It was shown in Zetsche et al., Cell, 163, 759-771, 2015 (which is incorporated herein by reference) that, the RuvC-like domain of Cpf1 is responsible for cleaving both DNA strands and inactivation of the RuvC-like domain inactivates Cpf1 nuclease activity. For example, mutations corresponding to D917A, E1006A, or D1255A in Francisella novicida Cpf1 inactivate Cpf1 nuclease activity. In some embodiments, the dCpf1 of the present disclosure comprises mutations corresponding to D917A, E1006A, D1255A, D917A/E1006A, D917A/D1255A, E1006A/D1255A, or D917A/E1006A/D1255A. It is to be understood that any mutations, e.g., substitution mutations, deletions, or insertions that inactivate the RuvC domain of Cpf1, may be used in accordance with the present disclosure.

In some embodiments, the nucleic acid programmable nucleotide binding protein of any of the fusion proteins provided herein may be a Cpf1 protein. In some embodiments, the Cpf1 protein is a Cpf1 nickase (nCpf1). In some embodiments, the Cpf1 protein is a nuclease inactive Cpf1 (dCpf1). In some embodiments, the Cpf1, the nCpf1, or the dCpf1 comprises an amino acid sequence that is at least 85%, at least 90%, at least 91%, at least 92%, at least 93%, at least 94%, at least 95%, at least 96%, at least 97%, at least 98%, at least 99%, or at least 99.5% identical to a Cpf1 sequence disclosed herein. In some embodiments, the dCpf1 comprises an amino acid sequence that is at least 85%, at least 90%, at least 91%, at least 92%, at least 93%, at least 94%, at least 95%, at least 96%, at least 97%, at least 98%, at least 99%, or at ease 99.5% identical to a Cpf1 sequence disclosed herein, and comprises mutations corresponding to D917A, E1006A, D1255A, D917A/E1006A, D917A/D1255A, E1006A/D1255A, or D917A/E1006A/D1255A. It should be appreciated that Cpf1 from other bacterial species may also be used in accordance with the present disclosure.

The amino acid sequence of wild type Francisella novicida Cpf1 follows. D917, E1006, and D1255 are bolded and underlined.

MSIYQEFVNKYSLSKTLRFELIPQGKTLENIKARGLILDDEKRAKDYKKA
KQIIDKYHQFFIEEILSSVCISEDLLQNYSDVYFKLKKSDDDNLQKDFKS
AKDTIKKQISEYIKDSEKFKNLFNQNLIDAKKGQESDLILWLKQSKDNGI
ELFKANSDITDIDEALEIIKSFKGWTTYFKGFHENRKNVYSSNDIPTSII
YRIVDDNLPKFLENKAKYESLKDKAPEAINYEQIKKDLAEELTFDIDYKT
SEVNQRVFSLDEVFEIANFNNYLNQSGITKFNTIIGGKFVNGENTKRKGI
NEYINLYSQQINDKTLKKYKMSVLFKQILSDTESKSFVIDKLEDDSDVVT
TMQSFYEQIAAFKTVEEKSIKETLSLLFDDLKAQKLDLSKIYFKNDKSLT
DLSQQVFDDYSVIGTAVLEYITQQIAPKNLDNPSKKEQELIAKKTEKAKY
LSLETIKLALEEFNKHRDIDKQCRFEEILANFAAIPMIFDEIAQNKDNLA
QISIKYQNQGKKDLLQASAEDDVKAIKDLLDQTNNLLHKLKIFHISQSED
KANILDKDEHFYLVFEECYFELANIVPLYNKIRNYITQKPYSDEKFKLNF
ENSTLANGWDKNKEPDNTAILFIKDDKYYLGVMNKKNNKIFDDKAIKENK
GEGYKKIVYKLLPGANKMLPKVFFSAKSIKFYNPSEDILRIRNHSTHTKN
GSPQKGYEKFEFNIEDCRKFIDFYKQSISKHPEWKDFGFRFSDTQRYNSI
DEFYREVENQGYKLTFENISESYIDSVVNQGKLYLFQIYNKDFSAYSKGR
PNLHTLYWKALFDERNLQDVVYKLNGEAELFYRKQSIPKKITHPAKEAIA
NKNKDNPKKESVFEYDLIKDKRFTEDKFFFHCPITINFKSSGANKFNDEI
NLLLKEKANDVHILSIDRGERHLAYYTLVDGKGNIIKQDTFNIIGNDRMK
TNYHDKLAAIEKDRDSARKDWKKINNIKEMKEGYLSQVVHEIAKLVIEYN
AIVVFEDLNFGFKRGRFKVEKQVYQKLEKMLIEKLNYLVFKDNEFDKTGG
VLRAYQLTAPFETFKKMGKQTGIIYYVPAGFTSKICPVTGFVNQLYPKYE
SVSKSQEFFSKFDKICYNLDKGYFEFSFDYKNFGDKAAKGKWTIASFGSR
LINFRNSDKNHNWDTREVYPTKELEKLLKDYSIEYGHGECIKAAICGESD
KKFFAKLTSVLNTILQMRNSKTGTELDYLISPVADVNGNFFDSRQAPKNM
PQDADANGAYHIGLKGLMLLGRIKNNQEGKKLNLVIKNEEYFEFVQNRN
N.

The amino acid sequence of Francisella novicida Cpf1 D917A follows. (A917, E1006, and D1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIARGERHLAYYTLVDG
KGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDADANGAYHIGLKGLMLLGRIK
NNQEGKKLNLVIKNEEYFEFVQNRNN.

The amino acid sequence of Francisella novicida Cpf1 E1006A follows. (D917, A1006, and D1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIDRGERHLAYYTLVDG
KGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDADANGAYHIGLKGLMLLGRIK
NNQEGKKLNLVIKNEEYFEFVQNRNN.

The amino acid sequence of Francisella novicida Cpf1 D1255A follows. (D917, E1006 and A1255 mutation positions are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIDRGERHLAYYTLVDG
KGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDAAANGAYHIGLKGLMLLGRIK
NNQEGKKLNLVIKNEEYFEFVQNRNN

The amino acid sequence of Francisella novicida Cpf1 D917A/E1006A follows. (A917, A1006, and D1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIARGERHLAYYTLVDG
KGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDADANGAYHIGLKGLMLLGRIK
NNQEGKKLNLVIKNEEYFEFVQNRNN.

The amino acid sequence of Francisella novicida Cpf1 D917A/D1255A follows. (A917, E1006, and A1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIARGERHLAYYTLVDG
KGNIIKQDTFNIIGNDRMKTNYHDKLAAIEKDRDSARKDWKKINNIKEM
KEGYLSQVVHEIAKLVIEYNAIVVFEDLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDAAANGAYHIGLKGLMLLGRIK
NNQEGKKLNLVIKNEEYFEFVQNRNN.

The amino acid sequence of Francisella novicida Cpf1 E1006A/D1255A follows. (D917, A1006, and A1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIDRGERHLAYYTLVDG
KGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDAAANGAYHIGLKGLMLLGRIK
NNQEGKKLNLVIKNEEYFEFVQNRNN.

The amino acid sequence of Francisella novicida Cpf1 D917A/E1006A/D1255A follows. (A917, A1006, and A1255 are bolded and underlined).

MSIYQEFVNKYSLSKTLRFELIPQGKTLENIKARGLILDDEKRAKDYKK
AKQIIDKYHQFFIEEILSSVCISEDLLQNYSDVYFKLKKSDDDNLQKDF
KSAKDTIKKQISEYIKDSEKFKNLFNQNLIDAKKGQESDLILWLKQSKD
NGIELFKANSDITDIDEALEIIKSFKGWTTYFKGFHENRKNVYSSNDIP
TSIIYRIVDDNLPKFLENKAKYESLKDKAPEAINYEQIKKDLAEELTFD
IDYKTSEVNQRVFSLDEVFEIANFNNYLNQSGITKFNTIIGGKFVNGEN
TKRKGINEYINLYSQQINDKTLKKYKMSVLFKQILSDTESKSFVIDKLE
DDSDVVTTMQSFYEQIAAFKTVEEKSIKETLSLLFDDLKAQKLDLSKIY
FKNDKSLTDLSQQVFDDYSVIGTAVLEYITQQIAPKNLDNPSKKEQELI
AKKTEKAKYLSLETIKLALEEFNKHRDIDKQCRFEEILANFAAIPMIFD
EIAQNKDNLAQISIKYQNQGKKDLLQASAEDDVKAIKDLLDQTNNLLHK
LKIFHISQSEDKANILDKDEHFYLVFEECYFELANIVPLYNKIRNYITQ
KPYSDEKFKLNFENSTLANGWDKNKEPDNTAILFIKDDKYYLGVMNKKN
NKIFDDKAIKENKGEGYKKIVYKLLPGANKMLPKVFFSAKSIKFYNPSE
DILRIRNHSTHTKNGSPQKGYEKFEFNIEDCRKFIDFYKQSISKHPEWK
DFGFRFSDTQRYNSIDEFYREVENQGYKLTFENISESYIDSVVNQGKLY
LFQIYNKDFSAYSKGRPNLHTLYWKALFDERNLQDVVYKLNGEAELFYR
KQSIPKKITHPAKEAIANKNKDNPKKESVFEYDLIKDKRFTEDKFFFHC
PITINFKSSGANKFNDEINLLLKEKANDVHILSIARGERHLAYYTLVDG
KGNIIKQDTFNIIGNDRMKTNYHDKLAAIEKDRDSARKDWKKINNIKEM
KEGYLSQVVHEIAKLVIEYNAIVVFADLNFGFKRGRFKVEKQVYQKLEK
MLIEKLNYLVFKDNEFDKTGGVLRAYQLTAPFETFKKMGKQTGIIYYVP
AGFTSKICPVTGFVNQLYPKYESVSKSQEFFSKFDKICYNLDKGYFEFS
FDYKNFGDKAAKGKWTIASFGSRLINFRNSDKNHNWDTREVYPTKELEK
LLKDYSIEYGHGECIKAAICGESDKKFFAKLTSVLNTILQMRNSKTGTE
LDYLISPVADVNGNFFDSRQAPKNMPQDAAANGAYHIGLKGLMLLGRIK
NNQEGKKLNLVIKNEEYFEFVQNRNN.

In some embodiments, one of the Cas9 domains present in the fusion protein may be replaced with a guide nucleotide sequence-programmable DNA-binding protein domain that has no requirements for a PAM sequence.

In some embodiments, the Cas domain is a Cas9 domain from Staphylococcus aureus (SaCas9). In some embodiments, the SaCas9 domain is a nuclease active SaCas9, a nuclease inactive SaCas9 (SaCas9d), or a SaCas9 nickase (SaCas9n). In some embodiments, the SaCas9 domain comprises a N579A mutation, or a corresponding mutation in any of the amino acid sequences provided herein.

In some embodiments, the SaCas9 domain, the SaCas9d domain, or the SaCas9n domain can bind to a nucleic acid sequence having a non-canonical PAM. In some embodiments, the SaCas9 domain, the SaCas9d domain, or the SaCas9n domain can bind to a nucleic acid sequence having a NNGRRT or a NNGRRT PAM sequence. In some embodiments, the SaCas9 domain comprises one or more of a E781X, a N967X, and a R1014X mutation, or a corresponding mutation in any of the amino acid sequences provided herein, wherein X is any amino acid. In some embodiments, the SaCas9 domain comprises one or more of a E781K, a N967K, and a R1014H mutation, or one or more corresponding mutation in any of the amino acid sequences provided herein. In some embodiments, the SaCas9 domain comprises a E781K, a N967K, or a R1014H mutation, or corresponding mutations in any of the amino acid sequences provided herein.

The amino acid sequence of an exemplary SaCas9 is as follows:

MKRNYILGLDIGITSVGYGIIDYETRDVIDAGVRLFKEANVENNEGRRS
KRGARRLKRRRRHRIQRVKKLLFDYNLLTDHSELSGINPYEARVKGLSQ
KLSEEEFSAALLHLAKRRGVHNVNEVEEDTGNELSTKEQISRNSKALEE
KYVAELQLERLKKDGEVRGSINRFKTSDYVKEAKQLLKVQKAYHQLDQS
FIDTYIDLLETRRTYYEGPGEGSPFGWKDIKEWYEMLMGHCTYFPEELR
SVKYAYNADLYNALNDLNNLVITRDENEKLEYYEKFQIIENVFKQKKKP
TLKQIAKEILVNEEDIKGYRVTSTGKPEFTNLKVYHDIKDITARKEIIE
NAELLDQIAKILTIYQSSEDIQEELTNLNSELTQEEIEQISNLKGYTGT
HNLSLKAINLILDELWHTNDNQIAIFNRLKLVPKKVDLSQQKEIPTTLV
DDFILSPVVKRSFIQSIKVINAIIKKYGLPNDIIIELAREKNSKDAQKM
INEMQKRNRQTNERIEEIIRTTGKENAKYLIEKIKLHDMQEGKCLYSLE
AIPLEDLLNNPFNYEVDHIIPRSVSFDNSFNNKVLVKQEENSKKGNRTP
FQYLSSSDSKISYETFKKHILNLAKGKGRISKTKKEYLLEERDINRFSV
QKDFINRNLVDTRYATRGLMNLLRSYFRVNNLDVKVKSINGGFTSFLRR
KWKFKKERNKGYKHHAEDALIIANADFIFKEWKKLDKAKKVMENQMFEE
KQAESMPEIETEQEYKEIFITPHQIKHIKDFKDYKYSHRVDKKPNRELI
NDTLYSTRKDDKGNTLIVNNLNGLYDKDNDKLKKLINKSPEKLLMYHHD
PQTYQKLKLIMEQYGDEKNPLYKYYEETGNYLTKYSKKDNGPVIKKIKY
YGNKLNAHLDITDDYPNSRNKVVKLSLKPYRFDVYLDNGVYKFVTVKNL
DVIKKENYYEVNSKCYEEAKKLKKISNQAEFIASFYNNDLIKINGELYR
VIGVNNDLLNRIEVNMIDITYREYLENMNDKRPPRIIKTIASKTQSIKK
YSTDILGNLYEVKSKKHPQIIKKG.

In this sequence, residue N579, which is underlined and in bold, may be mutated (e.g., to a A579) to yield a SaCas9 nickase.

The amino acid sequence of an exemplary SaCas9n is as follows:

KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEEASKKGNRTPF
QYLSSSDSKISYETFKKHILNLAKGKGRISKIKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRELIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYNNDLIKINGELYRV
IGVNNDLLNRIEVNMIDITYREYLENMNDKRPPRIIKTIASKTQSIKKY
STDILGNLYEVKSKKHPQIIKKG.

In this sequence, residue A579, which can be mutated from N579 to yield a SaCas9 nickase, is underlined and in bold.

The amino acid sequences of an exemplary SaKKH Cas9 is as follows:

KRNYILGLDIGITSVGYGIIDYETRDVIDAGVRLFKEANVENNEGRRSK
RGARRLKRRRRHRIQRVKKLLFDYNLLTDHSELSGINPYEARVKGLSQK
LSEEEFSAALLHLAKRRGVHNVNEVEEDTGNELSTKEQISRNSKALEEK
YVAELQLERLKKDGEVRGSINRFKTSDYVKEAKQLLKVQKAYHQLDQSF
IDTYIDLLETRRTYYEGPGEGSPFGWKDIKEWYEMLMGHCTYFPEELRS
VKYAYNADLYNALNDLNNLVITRDENEKLEYYEKFQIIENVFKQKKKPT
LKQIAKEILVNEEDIKGYRVTSTGKPEFTNLKVYHDIKDITARKEIIEN
AELLDQIAKILTIYQSSEDIQEELTNLNSELTQEEIEQISNLKGYTGTH
NLSLKAINLILDELWHTNDNQIAIFNRLKLVPKKVDLSQQKEIPTTLVD
DFILSPVVKRSFIQSIKVINAIIKKYGLPNDIIIELAREKNSKDAQKMI
NEMQKRNRQTNERIEEIIRTTGKENAKYLIEKIKLHDMQEGKCLYSLEA
IPLEDLLNNPFNYEVDHIIPRSVSFDNSFNNKVLVKQEEASKKGNRTPF
QYLSSSDSKISYETFKKHILNLAKGKGRISKIKKEYLLEERDINRFSVQ
KDFINRNLVDTRYATRGLMNLLRSYFRVNNLDVKVKSINGGFTSFLRRK
WKFKKERNKGYKHHAEDALIIANADFIFKEWKKLDKAKKVMENQMFEEK
QAESMPEIETEQEYKEIFITPHQIKHIKDFKDYKYSHRVDKKPNRKLIN
DTLYSTRKDDKGNTLIVNNLNGLYDKDNDKLKKLINKSPEKLLMYHHDP
QTYQKLKLIMEQYGDEKNPLYKYYEETGNYLTKYSKKDNGPVIKKIKYY
GNKLNAHLDITDDYPNSRNKVVKLSLKPYRFDVYLDNGVYKFVTVKNLD
VIKKENYYEVNSKCYEEAKKLKKISNQAEFIASFYKNDLIKINGELYRV
IGVNNDLLNRIEVNMIDITYREYLENMNDKRPPHIIKTIASKTQSIKKY
STDILGNLYEVKSKKHPQIIKKG.

Residue A579 above, which can be mutated from N579 to yield a SaCas9 nickase, is underlined and in bold. Residues K781, K967, and H1014 above, which can be mutated from E781, N967, and R1014 to yield a SaKKH Cas9 are underlined and in italics.

### High Fidelity Cas9 Domains

Some aspects of the disclosure provide high fidelity Cas9 domains. In some embodiments, high fidelity Cas9 domains are engineered Cas9 domains comprising one or more mutations that decrease electrostatic interactions between the Cas9 domain and the sugar-phosphate backbone of a DNA, relative to a corresponding wild-type Cas9 domain. High fidelity Cas9 domains that have decreased electrostatic interactions with the sugar-phosphate backbone of DNA can have less off-target effects. In some embodiments, the Cas9 domain (e.g., a wild type Cas9 domain) comprises one or more mutations that decrease the association between the Cas9 domain and the sugar-phosphate backbone of a DNA. In some embodiments, a Cas9 domain comprises one or more mutations that decreases the association between the Cas9 domain and the sugar-phosphate backbone of DNA by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, or at least 70%.

In some embodiments, any of the Cas9 fusion proteins provided herein comprise one or more of a N497X, a R661X, a Q695X, and/or a Q926X mutation, or a corresponding mutation in any of the amino acid sequences provided herein, wherein X is any amino acid. In some embodiments, any of the Cas9 fusion proteins provided herein comprise one or more of a N497A, a R661A, a Q695A, and/or a Q926A mutation, or a corresponding mutation in any of the amino acid sequences provided herein. In some embodiments, the Cas9 domain comprises a D10A mutation, or a corresponding mutation in any of the amino acid sequences provided herein. Cas9 domains with high fidelity are known in the art and would be apparent to the skilled artisan. For example, Cas9 domains with high fidelity have been described in Kleinstiver, B. P., et al. “High-fidelity CRISPR-Cas9 nucleases with no detectable genome-wide off-target effects.” Nature 529, 490-495 (2016); and Slaymaker, I. M., et al. “Rationally engineered Cas9 nucleases with improved specificity.” Science 351, 84-88 (2015); the entire contents of each are incorporated herein by reference.

In some embodiments, the modified Cas9 is a high fidelity Cas9 enzyme. In some embodiments, the high fidelity Cas9 enzyme is SpCas9 (K855A), eSpCas9(1.1), SpCas9-HF1, or hyper accurate Cas9 variant (HypaCas9). The modified Cas9 eSpCas9(1.1) contains alanine substitutions that weaken the interactions between the HNH/RuvC groove and the non-target DNA strand, preventing strand separation and cutting at off-target sites. Similarly, SpCas9-HF1 lowers off-target editing through alanine substitutions that disrupt Cas9's interactions with the DNA phosphate backbone. HypaCas9 contains mutations (SpCas9 N692A/M694A/Q695A/H698A) in the REC3 domain that increase Cas9 proofreading and target discrimination. All three high fidelity enzymes generate less off-target editing than wildtype Cas9.

An exemplary high fidelity Cas9 is provided below.

High Fidelity Cas9 domain mutations relative to Cas9 are shown in bold and underline

MDKKYSIGL [symbol] IGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMT [symbol] FDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWG [symbol] LSRKLINGIRDKQSGKTILDFLKSDGFANRNFM [symbol] LIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETR [symbol] ITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

### Guide Polynucleotides

In an embodiment, the guide polynucleotide is a guide RNA. An RNA/Cas complex can assist in “guiding” Cas protein to a target DNA. Cas9/crRNA/tracrRNA endonucleolytically cleaves linear or circular dsDNA target complementary to the spacer. The target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species. See, e.g., Jinek M. et al., Science 337:816-821(2012), the entire contents of which is hereby incorporated by reference. Cas9 recognizes a short motif in the CRISPR repeat sequences (the PAM or protospacer adjacent motif) to help distinguish self-versus-non self. Cas9 nuclease sequences and structures are well known to those of skill in the art (see e.g., “Complete genome sequence of an M1 strain of Streptococcus pyogenes.” Ferretti, J. J. et al., Natl. Acad. Sci. U.S.A. 98:4658-4663(2001); “CRISPR RNA maturation by trans-encoded small RNA and host factor RNase III.” Deltcheva E. et al., Nature 471:602-607(2011); and “Programmable dual-RNA-guided DNA endonuclease in adaptive bacterial immunity.” Jinek M. et al, Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences can be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference. In some embodiments, a Cas9 nuclease has an inactive (e.g., an inactivated) DNA cleavage domain, that is, the Cas9 is a nickase.

In some embodiments, the guide polynucleotide is at least one single guide RNA (“sgRNA” or “gNRA”). In some embodiments, the guide polynucleotide is at least one tracrRNA. In some embodiments, the guide polynucleotide does not require PAM sequence to guide the polynucleotide-programmable DNA-binding domain (e.g., Cas9 or Cpf1) to the target nucleotide sequence.

The polynucleotide programmable nucleotide binding domain (e.g., a CRISPR-derived domain) of the base editors disclosed herein can recognize a target polynucleotide sequence by associating with a guide polynucleotide. A guide polynucleotide (e.g., gRNA) is typically single-stranded and can be programmed to site-specifically bind (i.e., via complementary base pairing) to a target sequence of a polynucleotide, thereby directing a base editor that is in conjunction with the guide nucleic acid to the target sequence. A guide polynucleotide can be DNA. A guide polynucleotide can be RNA. In some cases, the guide polynucleotide comprises natural nucleotides (e.g., adenosine). In some cases, the guide polynucleotide comprises non-natural (or unnatural) nucleotides (e.g., peptide nucleic acid or nucleotide analogs). In some cases, the targeting region of a guide nucleic acid sequence can be at least 15, 16, 17, 18, 19, 20, 21, 22, 23, 24, 25, 26, 27, 28, 29, or 30 nucleotides in length. A targeting region of a guide nucleic acid can be between 10-30 nucleotides in length, or between 15-25 nucleotides in length, or between 15-20 nucleotides in length.

In some embodiments, a guide polynucleotide comprises two or more individual polynucleotides, which can interact with one another via for example complementary base pairing (e.g., a dual guide polynucleotide). For example, a guide polynucleotide can comprise a CRISPR RNA (crRNA) and a trans-activating CRISPR RNA (tracrRNA). For example, a guide polynucleotide can comprise one or more trans-activating CRISPR RNA (tracrRNA).

In type II CRISPR systems, targeting of a nucleic acid by a CRISPR protein (e.g., Cas9) typically requires complementary base pairing between a first RNA molecule (crRNA) comprising a sequence that recognizes the target sequence and a second RNA molecule (trRNA) comprising repeat sequences which forms a scaffold region that stabilizes the guide RNA-CRISPR protein complex. Such dual guide RNA systems can be employed as a guide polynucleotide to direct the base editors disclosed herein to a target polynucleotide sequence.

In some embodiments, the base editor provided herein utilizes a single guide polynucleotide (e.g., gRNA). In some embodiments, the base editor provided herein utilizes a dual guide polynucleotide (e.g., dual gRNAs). In some embodiments, the base editor provided herein utilizes one or more guide polynucleotide (e.g., multiple gRNA). In some embodiments, a single guide polynucleotide is utilized for different base editors described herein. For example, a single guide polynucleotide can be utilized for a cytidine base editor and an adenosine base editor.

In other embodiments, a guide polynucleotide can comprise both the polynucleotide targeting portion of the nucleic acid and the scaffold portion of the nucleic acid in a single molecule (i.e., a single-molecule guide nucleic acid). For example, a single-molecule guide polynucleotide can be a single guide RNA (sgRNA or gRNA). Herein the term guide polynucleotide sequence contemplates any single, dual, or multi-molecule nucleic acid capable of interacting with and directing a base editor to a target polynucleotide sequence.

Typically, a guide polynucleotide (e.g., crRNA/trRNA complex or a gRNA) comprises a “polynucleotide-targeting segment” that includes a sequence capable of recognizing and binding to a target polynucleotide sequence, and a “protein-binding segment” that stabilizes the guide polynucleotide within a polynucleotide programmable nucleotide binding domain component of a base editor. In some embodiments, the polynucleotide targeting segment of the guide polynucleotide recognizes and binds to a DNA polynucleotide, thereby facilitating the editing of a base in DNA. In other cases, the polynucleotide targeting segment of the guide polynucleotide recognizes and binds to an RNA polynucleotide, thereby facilitating the editing of a base in RNA. Herein a “segment” refers to a section or region of a molecule, e.g., a contiguous stretch of nucleotides in the guide polynucleotide. A segment can also refer to a region/section of a complex such that a segment can comprise regions of more than one molecule. For example, where a guide polynucleotide comprises multiple nucleic acid molecules, the protein-binding segment of can include all or a portion of multiple separate molecules that are for instance hybridized along a region of complementarity. In some embodiments, a protein-binding segment of a DNA-targeting RNA that comprises two separate molecules can comprise (i) base pairs 40-75 of a first RNA molecule that is 100 base pairs in length; and (ii) base pairs 10-25 of a second RNA molecule that is 50 base pairs in length. The definition of “segment,” unless otherwise specifically defined in a particular context, is not limited to a specific number of total base pairs, is not limited to any particular number of base pairs from a given RNA molecule, is not limited to a particular number of separate molecules within a complex, and can include regions of RNA molecules that are of any total length and can include regions with complementarity to other molecules.

A guide RNA or a guide polynucleotide can comprise two or more RNAs, e.g., CRISPR RNA (crRNA) and transactivating crRNA (tracrRNA). A guide RNA or a guide polynucleotide can sometimes comprise a single-chain RNA, or single guide RNA (sgRNA) formed by fusion of a portion (e.g., a functional portion) of crRNA and tracrRNA. A guide RNA or a guide polynucleotide can also be a dual RNA comprising a crRNA and a tracrRNA. Furthermore, a crRNA can hybridize with a target DNA.

As discussed above, a guide RNA or a guide polynucleotide can be an expression product. For example, a DNA that encodes a guide RNA can be a vector comprising a sequence coding for the guide RNA. A guide RNA or a guide polynucleotide can be transferred into a cell by transfecting the cell with an isolated guide RNA or plasmid DNA comprising a sequence coding for the guide RNA and a promoter. A guide RNA or a guide polynucleotide can also be transferred into a cell in other way, such as using virus-mediated gene delivery.

A guide RNA or a guide polynucleotide can be isolated. For example, a guide RNA can be transfected in the form of an isolated RNA into a cell or organism. A guide RNA can be prepared by in vitro transcription using any in vitro transcription system known in the art. A guide RNA can be transferred to a cell in the form of isolated RNA rather than in the form of plasmid comprising encoding sequence for a guide RNA.

A guide RNA or a guide polynucleotide can comprise three regions: a first region at the 5′ end that can be complementary to a target site in a chromosomal sequence, a second internal region that can form a stem loop structure, and a third 3′ region that can be single-stranded. A first region of each guide RNA can also be different such that each guide RNA guides a fusion protein to a specific target site. Further, second and third regions of each guide RNA can be identical in all guide RNAs.

A first region of a guide RNA or a guide polynucleotide can be complementary to sequence at a target site in a chromosomal sequence such that the first region of the guide RNA can base pair with the target site. In some cases, a first region of a guide RNA can comprise from or from about 10 nucleotides to 25 nucleotides (i.e., from 10 nucleotides to nucleotides; or from about 10 nucleotides to about 25 nucleotides; or from 10 nucleotides to about 25 nucleotides; or from about 10 nucleotides to 25 nucleotides) or more. For example, a region of base pairing between a first region of a guide RNA and a target site in a chromosomal sequence can be or can be about 10, 11, 12, 13, 14, 15, 16, 17, 18, 19, 20, 22, 23, 24, 25, or more nucleotides in length. Sometimes, a first region of a guide RNA can be or can be about 19, 20, or 21 nucleotides in length.

A guide RNA or a guide polynucleotide can also comprise a second region that forms a secondary structure. For example, a secondary structure formed by a guide RNA can comprise a stem (or hairpin) and a loop. A length of a loop and a stem can vary. For example, a loop can range from or from about 3 to 10 nucleotides in length, and a stem can range from or from about 6 to 20 base pairs in length. A stem can comprise one or more bulges of 1 to 10 or about 10 nucleotides. The overall length of a second region can range from or from about 16 to 60 nucleotides in length. For example, a loop can be or can be about 4 nucleotides in length and a stem can be or can be about 12 base pairs.

A guide RNA or a guide polynucleotide can also comprise a third region at the 3′ end that can be essentially single-stranded. For example, a third region is sometimes not complementarity to any chromosomal sequence in a cell of interest and is sometimes not complementarity to the rest of a guide RNA. Further, the length of a third region can vary. A third region can be more than or more than about 4 nucleotides in length. For example, the length of a third region can range from or from about 5 to 60 nucleotides in length.

A guide RNA or a guide polynucleotide can target any exon or intron of a gene target. In some cases, a guide can target exon 1 or 2 of a gene, in other cases; a guide can target exon 3 or 4 of a gene. A composition can comprise multiple guide RNAs that all target the same exon or in some cases, multiple guide RNAs that can target different exons. An exon and an intron of a gene can be targeted.

A guide RNA or a guide polynucleotide can target a nucleic acid sequence of or of about 20 nucleotides. A target nucleic acid can be less than or less than about 20 nucleotides. A target nucleic acid can be at least or at least about 5, 10, 15, 16, 17, 18, 19, 20, 21, 22, 23, 24, 25, 30, or anywhere between 1-100 nucleotides in length. A target nucleic acid can be at most or at most about 5, 10, 15, 16, 17, 18, 19, 20, 21, 22, 23, 24, 25, 30, 40, 50, or anywhere between 1-100 nucleotides in length. A target nucleic acid sequence can be or can be about 20 bases immediately 5′ of the first nucleotide of the PAM. A guide RNA can target a nucleic acid sequence. A target nucleic acid can be at least or at least about 1-10, 1-20, 1-30, 1-40, 1-50, 1-60, 1-70, 1-80, 1-90, or 1-100 nucleotides.

A guide polynucleotide, for example, a guide RNA, can refer to a nucleic acid that can hybridize to another nucleic acid, for example, the target nucleic acid or protospacer in a genome of a cell. A guide polynucleotide can be RNA. A guide polynucleotide can be DNA. The guide polynucleotide can be programmed or designed to bind to a sequence of nucleic acid site-specifically. A guide polynucleotide can comprise a polynucleotide chain and can be called a single guide polynucleotide. A guide polynucleotide can comprise two polynucleotide chains and can be called a double guide polynucleotide. A guide RNA can be introduced into a cell or embryo as an RNA molecule. For example, a RNA molecule can be transcribed in vitro and/or can be chemically synthesized. An RNA can be transcribed from a synthetic DNA molecule, e.g., a gBlocks® gene fragment. A guide RNA can then be introduced into a cell or embryo as an RNA molecule. A guide RNA can also be introduced into a cell or embryo in the form of a non-RNA nucleic acid molecule, e.g., DNA molecule. For example, a DNA encoding a guide RNA can be operably linked to promoter control sequence for expression of the guide RNA in a cell or embryo of interest. A RNA coding sequence can be operably linked to a promoter sequence that is recognized by RNA polymerase III (Pol III). Plasmid vectors that can be used to express guide RNA include, but are not limited to, px330 vectors and px333 vectors. In some cases, a plasmid vector (e.g., px333 vector) can comprise at least two guide RNA-encoding DNA sequences.

Methods for selecting, designing, and validating guide polynucleotides, e.g., guide RNAs and targeting sequences are described herein and known to those skilled in the art. For example, to minimize the impact of potential substrate promiscuity of a deaminase domain in the nucleobase editor system (e.g., an AID domain), the number of residues that could unintentionally be targeted for deamination (e.g., off-target C residues that could potentially reside on ssDNA within the target nucleic acid locus) may be minimized. In addition, software tools can be used to optimize the gRNAs corresponding to a target nucleic acid sequence, e.g., to minimize total off-target activity across the genome. For example, for each possible targeting domain choice using S. pyogenes Cas9, all off-target sequences (preceding selected PAMs, e.g., NAG or NGG) may be identified across the genome that contain up to certain number (e.g., 1, 2, 3, 4, 5, 6, 7, 8, 9, or 10) of mismatched base-pairs. First regions of gRNAs complementary to a target site can be identified, and all first regions (e.g., crRNAs) can be ranked according to its total predicted off-target score; the top-ranked targeting domains represent those that are likely to have the greatest on-target and the least off-target activity. Candidate targeting gRNAs can be functionally evaluated by using methods known in the art and/or as set forth herein.

As a non-limiting example, target DNA hybridizing sequences in crRNAs of a guide RNA for use with Cas9s may be identified using a DNA sequence searching algorithm. gRNA design may be carried out using custom gRNA design software based on the public tool cas-offinder as described in Bae S., Park J., & Kim J.-S. Cas-OFFinder: A fast and versatile algorithm that searches for potential off-target sites of Cas9 RNA-guided endonucleases. Bioinformatics 30, 1473-1475 (2014). This software scores guides after calculating their genome-wide off-target propensity. Typically matches ranging from perfect matches to 7 mismatches are considered for guides ranging in length from 17 to 24. Once the off-target sites are computationally-determined, an aggregate score is calculated for each guide and summarized in a tabular output using a web-interface. In addition to identifying potential target sites adjacent to PAM sequences, the software also identifies all PAM adjacent sequences that differ by 1, 2, 3 or more than 3 nucleotides from the selected target sites. Genomic DNA sequences for a target nucleic acid sequence, e.g., a target gene may be obtained and repeat elements may be screened using publicly available tools, for example, the RepeatMasker program. RepeatMasker searches input DNA sequences for repeated elements and regions of low complexity. The output is a detailed annotation of the repeats present in a given query sequence.

Following identification, first regions of guide RNAs, e.g., crRNAs, may be ranked into tiers based on their distance to the target site, their orthogonality and presence of 5′ nucleotides for close matches with relevant PAM sequences (for example, a 5′ G based on identification of close matches in the human genome containing a relevant PAM e.g., NGG PAM for S. pyogenes, NNGRRT or NNGRRV PAM for S. aureus). As used herein, orthogonality refers to the number of sequences in the human genome that contain a minimum number of mismatches to the target sequence. A “high level of orthogonality” or “good orthogonality” may, for example, refer to 20-mer targeting domains that have no identical sequences in the human genome besides the intended target, nor any sequences that contain one or two mismatches in the target sequence. Targeting domains with good orthogonality may be selected to minimize off-target DNA cleavage.

In some embodiments, a reporter system may be used for detecting base-editing activity and testing candidate guide polynucleotides. In some embodiments, a reporter system may comprise a reporter gene based assay where base editing activity leads to expression of the reporter gene. For example, a reporter system may include a reporter gene comprising a deactivated start codon, e.g., a mutation on the template strand from 3′-TAC-5′ to 3′-CAC-5′. Upon successful deamination of the target C, the corresponding mRNA will be transcribed as 5′-AUG-3′ instead of 5′-GUG-3′, enabling the translation of the reporter gene. Suitable reporter genes will be apparent to those of skill in the art. Non-limiting examples of reporter genes include gene encoding green fluorescence protein (GFP), red fluorescence protein (RFP), luciferase, secreted alkaline phosphatase (SEAP), or any other gene whose expression are detectable and apparent to those skilled in the art. The reporter system can be used to test many different gRNAs, e.g., in order to determine which residue(s) with respect to the target DNA sequence the respective deaminase will target. sgRNAs that target non-template strand can also be tested in order to assess off-target effects of a specific base editing protein, e.g., a Cas9 deaminase fusion protein. In some embodiments, such gRNAs can be designed such that the mutated start codon will not be base-paired with the gRNA. The guide polynucleotides can comprise standard ribonucleotides, modified ribonucleotides (e.g., pseudouridine), ribonucleotide isomers, and/or ribonucleotide analogs. In some embodiments, the guide polynucleotide can comprise at least one detectable label. The detectable label can be a fluorophore (e.g., FAM, TMR, Cy3, Cy5, Texas Red, Oregon Green, Alexa Fluors, Halo tags, or suitable fluorescent dye), a detection tag (e.g., biotin, digoxigenin, and the like), quantum dots, or gold particles.

The guide polynucleotides can be synthesized chemically, synthesized enzymatically, or a combination thereof. For example, the guide RNA can be synthesized using standard phosphoramidite-based solid-phase synthesis methods. Alternatively, the guide RNA can be synthesized in vitro by operably linking DNA encoding the guide RNA to a promoter control sequence that is recognized by a phage RNA polymerase. Examples of suitable phage promoter sequences include T7, T3, SP6 promoter sequences, or variations thereof. In embodiments in which the guide RNA comprises two separate molecules (e.g., crRNA and tracrRNA), the crRNA can be chemically synthesized and the tracrRNA can be enzymatically synthesized.

In some embodiments, a base editor system may comprise multiple guide polynucleotides, e.g., gRNAs. For example, the gRNAs may target to one or more target loci (e.g., at least 1 gRNA, at least 2 gRNA, at least 5 gRNA, at least 10 gRNA, at least 20 gRNA, at least 30 g RNA, at least 50 gRNA) comprised in a base editor system. The multiple gRNA sequences can be tandemly arranged and are preferably separated by a direct repeat.

A DNA sequence encoding a guide RNA (gRNA) or a guide polynucleotide can also be part of a vector. Further, a vector can comprise additional expression control sequences (e.g., enhancer sequences, Kozak sequences, polyadenylation sequences, transcriptional termination sequences, etc.), selectable marker sequences (e.g., GFP or antibiotic resistance genes such as puromycin), origins of replication, and the like. A DNA molecule encoding a guide RNA can also be linear. A DNA molecule encoding a guide RNA (gRNA) or a guide polynucleotide can also be circular.

In some embodiments, one or more components of a base editor system may be encoded by DNA sequences. Such DNA sequences may be introduced into an expression system, e.g., a cell, together or separately. For example, DNA sequences encoding a polynucleotide programmable nucleotide binding domain and a guide RNA may be introduced into a cell, each DNA sequence can be part of a separate molecule (e.g., one vector containing the polynucleotide programmable nucleotide binding domain coding sequence and a second vector containing the guide RNA coding sequence) or both can be part of a same molecule (e.g., one vector containing coding (and regulatory) sequence for both the polynucleotide programmable nucleotide binding domain and the guide RNA).

A guide polynucleotide can comprise one or more modifications to provide a nucleic acid with a new or enhanced feature. A guide polynucleotide can comprise a nucleic acid affinity tag. A guide polynucleotide can comprise synthetic nucleotide, synthetic nucleotide analog, nucleotide derivatives, and/or modified nucleotides.

In some cases, a gRNA or a guide polynucleotide can comprise modifications. A modification can be made at any location of a gRNA or a guide polynucleotide. More than one modification can be made to a single gRNA or a guide polynucleotide. A gRNA or a guide polynucleotide can undergo quality control after a modification. In some cases, quality control can include PAGE, HPLC, MS, or any combination thereof.

A modification of a gRNA or a guide polynucleotide can be a substitution, insertion, deletion, chemical modification, physical modification, stabilization, purification, or any combination thereof.

A gRNA or a guide polynucleotide can also be modified by 5′adenylate, 5′ guanosine-triphosphate cap, 5′N7-Methylguanosine-triphosphate cap, 5′triphosphate cap, 3′phosphate, 3′thiophosphate, 5′phosphate, 5′thiophosphate, Cis-Syn thymidine dimer, trimers, C12 spacer, C3 spacer, C6 spacer, dSpacer, PC spacer, rSpacer, Spacer 18, Spacer 9,3′-3′ modifications, 5′-5′ modifications, abasic, acridine, azobenzene, biotin, biotin BB, biotin TEG, cholesteryl TEG, desthiobiotin TEG, DNP TEG, DNP-X, DOTA, dT-Biotin, dual biotin, PC biotin, psoralen C2, psoralen C6, TINA, 3′DABCYL, black hole quencher 1, black hole quencer 2, DABCYL SE, dT-DABCYL, IRDye QC-1, QSY-21, QSY-35, QSY-7, QSY-9, carboxyl linker, thiol linkers, 2′-deoxyribonucleoside analog purine, 2′-deoxyribonucleoside analog pyrimidine, ribonucleoside analog, 2′-O-methyl ribonucleoside analog, sugar modified analogs, wobble/universal bases, fluorescent dye label, 2′-fluoro RNA, 2′-O-methyl RNA, methylphosphonate, phosphodiester DNA, phosphodiester RNA, phosphothioate DNA, phosphorothioate RNA, UNA, pseudouridine-5′-triphosphate, 5′-methylcytidine-5′-triphosphate, or any combination thereof.

In some cases, a modification is permanent. In other cases, a modification is transient. In some cases, multiple modifications are made to a gRNA or a guide polynucleotide. A gRNA or a guide polynucleotide modification can alter physiochemical properties of a nucleotide, such as their conformation, polarity, hydrophobicity, chemical reactivity, base-pairing interactions, or any combination thereof.

The PAM sequence can be any PAM sequence known in the art. Suitable PAM sequences include, but are not limited to, NGG, NGA, NGC, NGN, NGT, NGCG, NGAG, NGAN, NGNG, NGCN, NGCG, NGTN, NNGRRT, NNNRRT, NNGRR(N), TTTV, TYCV, TYCV, TATV, NNNNGATT, NNAGAAW, or NAAAAC. Y is a pyrimidine; N is any nucleotide base; W is A or T.

A modification can also be a phosphorothioate substitute. In some cases, a natural phosphodiester bond can be susceptible to rapid degradation by cellular nucleases and; a modification of internucleotide linkage using phosphorothioate (PS) bond substitutes can be more stable towards hydrolysis by cellular degradation. A modification can increase stability in a gRNA or a guide polynucleotide. A modification can also enhance biological activity. In some cases, a phosphorothioate enhanced RNA gRNA can inhibit RNase A, RNase T1, calf serum nucleases, or any combinations thereof. These properties can allow the use of PS-RNA gRNAs to be used in applications where exposure to nucleases is of high probability in vivo or in vitro. For example, phosphorothioate (PS) bonds can be introduced between the last 3-5 nucleotides at the 5′- or ″-end of a gRNA which can inhibit exonuclease degradation. In some cases, phosphorothioate bonds can be added throughout an entire gRNA to reduce attack by endonucleases.

### Protospacer Adjacent Motif

The term “protospacer adjacent motif (PAM)” or PAM-like motif refers to a 2-6 base pair DNA sequence immediately following the DNA sequence targeted by the Cas9 nuclease in the CRISPR bacterial adaptive immune system. In some embodiments, the PAM can be a 5′ PAM (i.e., located upstream of the 5′ end of the protospacer). In other embodiments, the PAM can be a 3′ PAM (i.e., located downstream of the 5′ end of the protospacer).

The PAM sequence is essential for target binding, but the exact sequence depends on a type of Cas protein.

A base editor provided herein can comprise a CRISPR protein-derived domain that is capable of binding a nucleotide sequence that contains a canonical or non-canonical protospacer adjacent motif (PAM) sequence. A PAM site is a nucleotide sequence in proximity to a target polynucleotide sequence. Some aspects of the disclosure provide for base editors comprising all or a portion of CRISPR proteins that have different PAM specificities. For example, typically Cas9 proteins, such as Cas9 from S. pyogenes (spCas9), require a canonical NGG PAM sequence to bind a particular nucleic acid region, where the “N” in “NGG” is adenine (A), thymine (T), guanine (G), or cytosine (C), and the G is guanine. A PAM can be CRISPR protein-specific and can be different between different base editors comprising different CRISPR protein-derived domains. A PAM can be 5′ or 3′ of a target sequence. A PAM can be upstream or downstream of a target sequence. A PAM can be 1, 2, 3, 4, 5, 6, 7, 8, 9, 10 or more nucleotides in length. Often, a PAM is between 2-6 nucleotides in length. Several PAM variants are described in Table 1 below.

TABLE 1
Cas9 proteins and corresponding PAM sequences

| Variant | PAM |
|---|---|
| spCas9 | NGG |
| spCas9-VRQR | NGA |
| spCas9-VRER | NGCG |
| spCas9-MQKFRAER | NGC |
| xCas9 (sp) | NGN |
| saCas9 | NNGRRT |
| saCas9-KKH | NNNRRT |
| spCas9-MQKSER | NGCG |
| spCas9-MQKSER | NGCN |
| spCas9-LRKIQK | NGTN |
| spCas9-LRVSQK | NGTN |
| spCas9-LRVSQL | NGTN |
| SpyMacCas9 | NAA |
| Cpf1 | 5′ (TTTV) |

In some embodiments, the PAM is NGC. In some embodiments, the NGC PAM is recognized by a Cas9 variant. In some embodiments, the NGC PAM variant includes one or more amino acid substitutions selected from D1135M, S1136Q, G1218K, E1219F, A1322R, D1332A, R1335E, and T1337R (collectively termed “MQKFRAER”).

In some embodiments, the PAM is NGT. In some embodiments, the NGT PAM is recognized by a Cas9 variant. In some embodiments, the NGT PAM variant is generated through targeted mutations at one or more residues 1335, 1337, 1135, 1136, 1218, and/or 1219. In some embodiments, the NGT PAM variant is created through targeted mutations at one or more residues 1219, 1335, 1337, 1218. In some embodiments, the NGT PAM variant is created through targeted mutations at one or more residues 1135, 1136, 1218, 1219, and 1335. In some embodiments, the NGT PAM variant is selected from the set of targeted mutations provided in Tables 2 and 3 below.

TABLE 2
NGT PAM Variant Mutations at residues 1219, 1335,
1337, 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |
| 9 | L | L | T | |
| 10 | L | L | R | |
| 11 | L | L | Q | |
| 12 | L | L | L | |
| 13 | F | I | T | |
| 14 | F | I | R | |
| 15 | F | I | Q | |
| 16 | F | I | L | |
| 17 | F | G | C | |
| 18 | H | L | N | |
| 19 | F | G | C | A |
| 20 | H | L | N | V |
| 21 | L | A | W | |
| 22 | L | A | F | |
| 23 | L | A | Y | |
| 24 | I | A | W | |
| 25 | I | A | F | |
| 26 | I | A | Y | |

TABLE 3
NGT PAM Variant Mutations at residues 1135, 1136,
1218, 1219, and 1335

| Variant | D1135L | S1136R | G1218S | E1219V | R1335Q |
|---|---|---|---|---|---|
| 27 | G | | | | |
| 28 | V | | | | |
| 29 | I | | | | |
| 30 | | A | | | |
| 31 | | W | | | |
| 32 | | H | | | |
| 33 | | K | | | |
| 34 | | | K | | |
| 35 | | | R | | |
| 36 | | | Q | | |
| 37 | | | T | | |
| 38 | | | N | | |
| 39 | | | | I | |
| 40 | | | | A | |
| 41 | | | | N | |
| 42 | | | | Q | |
| 43 | | | | G | |
| 44 | | | | L | |
| 45 | | | | S | |
| 46 | | | | T | |
| 47 | | | | | L |
| 48 | | | | | I |
| 49 | | | | | V |
| 50 | | | | | N |
| 51 | | | | | S |
| 52 | | | | | T |
| 53 | | | | | F |
| 54 | | | | | Y |
| 55 | N1286Q | I1331F | | | |

In some embodiments, the NGT PAM variant is selected from variant 5, 7, 28, 31, or 36 in Tables 2 and 3. In some embodiments, the variants have improved NGT PAM recognition.

In some embodiments, the NGT PAM variants have mutations at residues 1219, 1335, 1337, and/or 1218. In some embodiments, the NGT PAM variant is selected with mutations for improved recognition from the variants provided in Table 4 below.

TABLE 4
NGT PAM Variant Mutations at residues 1219, 1335,
1337, and 1218

| Variant | E1219V | R1335Q | T1337 | G1218 |
|---|---|---|---|---|
| 1 | F | V | T | |
| 2 | F | V | R | |
| 3 | F | V | Q | |
| 4 | F | V | L | |
| 5 | F | V | T | R |
| 6 | F | V | R | R |
| 7 | F | V | Q | R |
| 8 | F | V | L | R |

In some embodiments, the NGT PAM is selected from the variants provided in Table 5 below.

TABLE 5
NGT PAM variants

| | NGTN | | | | | | | |
|---|---|---|---|---|---|---|---|---|
| | variant | D1135 | S1136 | G1218 | E1219 | A1322R | R1335 | T1337 |
| Variant 1 | LRKIQK | L | R | K | I | — | Q | K |
| Variant 2 | LRSVQK | L | R | S | V | — | Q | K |
| Variant 3 | LRSVQL | L | R | S | V | — | Q | L |
| Variant 4 | LRKIRQK | L | R | K | I | R | Q | K |
| Variant 5 | LRSVRQK | L | R | S | V | R | Q | K |
| Variant 6 | LRSVRQL | L | R | S | V | R | Q | L |

In some embodiments the NGTN variant is variant 1. In some embodiments, the NGTN variant is variant 2. In some embodiments, the NGTN variant is variant 3. In some embodiments, the NGTN variant is variant 4. In some embodiments, the NGTN variant is variant 5. In some embodiments, the NGTN variant is variant 6.

In some embodiments, the Cas9 domain is a Cas9 domain from Streptococcus pyogenes (SpCas9). In some embodiments, the SpCas9 domain is a nuclease active SpCas9, a nuclease inactive SpCas9 (SpCas9d), or a SpCas9 nickase (SpCas9n). In some embodiments, the SpCas9 comprises a D9X mutation, or a corresponding mutation in any of the amino acid sequences provided herein, wherein X is any amino acid except for D. In some embodiments, the SpCas9 comprises a D9A mutation, or a corresponding mutation in any of the amino acid sequences provided herein. In some embodiments, the SpCas9 domain, the SpCas9d domain, or the SpCas9n domain can bind to a nucleic acid sequence having a non-canonical PAM. In some embodiments, the SpCas9 domain, the SpCas9d domain, or the SpCas9n domain can bind to a nucleic acid sequence having an NGG, a NGA, or a NGCG PAM sequence.

In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135E, R1335Q, and T1337R mutation, or a corresponding mutation in any of the amino acid sequences provided herein. In some embodiments, the SpCas9 domain comprises a D1135E, a R1335Q, and a T1337R mutation, or corresponding mutations in any of the amino acid sequences provided herein. In some embodiments, the SpCas9 domain comprises one or more of a D1135X, a R1335X, and a T1337X mutation, or a corresponding mutation in any of the amino acid sequences provided herein, wherein X is any amino acid. In some embodiments, the SpCas9 domain comprises one or more of a D1135V, a R1335Q, and a T1337R mutation, or a corresponding mutation in any of the amino acid sequences provided herein. In some embodiments, the SpCas9 domain comprises a D1135V, a R1335Q, and a T1337R mutation, or corresponding mutations in any of the amino acid sequences provided herein. In some embodiments, the SpCas9 domain comprises one or more of a D1135X, a G1218X, a R1335X, and a T1337X mutation, or a corresponding mutation in any of the amino acid sequences provided herein, wherein X is any amino acid. In some embodiments, the SpCas9 domain comprises one or more of a D1135V, a G1218R, a R1335Q, and a T1337R mutation, or a corresponding mutation in any of the amino acid sequences provided herein. In some embodiments, the SpCas9 domain comprises a D1135V, a G1218R, a R1335Q, and a T1337R mutation, or corresponding mutations in any of the amino acid sequences provided herein.

In some embodiments, the Cas9 domains of any of the fusion proteins provided herein comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to a Cas9 polypeptide described herein. In some embodiments, the Cas9 domains of any of the fusion proteins provided herein comprises the amino acid sequence of any Cas9 polypeptide described herein. In some embodiments, the Cas9 domains of any of the fusion proteins provided herein consists of the amino acid sequence of any Cas9 polypeptide described herein.

In some examples, a PAM recognized by a CRISPR protein-derived domain of a base editor disclosed herein can be provided to a cell on a separate oligonucleotide to an insert (e.g., an AAV insert) encoding the base editor. In such embodiments, providing PAM on a separate oligonucleotide can allow cleavage of a target sequence that otherwise would not be able to be cleaved, because no adjacent PAM is present on the same polynucleotide as the target sequence.

In an embodiment, S. pyogenes Cas9 (SpCas9) can be used as a CRISPR endonuclease for genome engineering. However, others can be used. In some embodiments, a different endonuclease can be used to target certain genomic targets. In some embodiments, synthetic SpCas9-derived variants with non-NGG PAM sequences can be used. Additionally, other Cas9 orthologues from various species have been identified and these “non-SpCas9s” can bind a variety of PAM sequences that can also be useful for the present disclosure. For example, the relatively large size of SpCas9 (approximately 4 kb coding sequence) can lead to plasmids carrying the SpCas9 cDNA that cannot be efficiently expressed in a cell. Conversely, the coding sequence for Staphylococcus aureus Cas9 (SaCas9) is approximately 1 kilobase shorter than SpCas9, possibly allowing it to be efficiently expressed in a cell. Similar to SpCas9, the SaCas9 endonuclease is capable of modifying target genes in mammalian cells in vitro and in mice in vivo. In some embodiments, a Cas protein can target a different PAM sequence. In some embodiments, a target gene can be adjacent to a Cas9 PAM, 5′-NGG, for example. In other embodiments, other Cas9 orthologs can have different PAM requirements. For example, other PAMs such as those of S. thermophilus (5′-NNAGAA for CRISPR1 and 5′-NGGNG for CRISPR3) and Neisseria meningiditis (5′-NNNNGATT) can also be found adjacent to a target gene.

In some embodiments, for a S. pyogenes system, a target gene sequence can precede (i.e., be 5′ to) a 5′-NGG PAM, and a 20-nt guide RNA sequence can base pair with an opposite strand to mediate a Cas9 cleavage adjacent to a PAM. In some embodiments, an adjacent cut can be or can be about 3 base pairs upstream of a PAM. In some embodiments, an adjacent cut can be or can be about 10 base pairs upstream of a PAM. In some embodiments, an adjacent cut can be or can be about 0-20 base pairs upstream of a PAM. For example, an adjacent cut can be next to, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 base pairs upstream of a PAM. An adjacent cut can also be downstream of a PAM by 1 to 30 base pairs. The sequences of exemplary SpCas9 proteins capable of binding a PAM sequence follow:

The amino acid sequence of an exemplary PAM-binding SpCas9 is as follows:

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

The amino acid sequence of an exemplary PAM-binding SpCas9n is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

The amino acid sequence of an exemplary PAM-binding SpEQR Cas9 is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFVEEDKKHERHPIFGNIVDEVAYHEKYPTIYHLRKKLVDSTDKADL
RLIYLALAHMIKFRGHFLIEGDLNPDNSDVDKLFIQLVQTYNQLFEENPI
NASGVDAKAILSARLSKSRRLENLIAQLPGEKKNGLFGNLIALSLGLTPN
FKSNFDLAEDAKLQLSKDTYDDDLDNLLAQIGDQYADLFLAAKNLSDAIL
LSDILRVNTEITKAPLSASMIKRYDEHHQDLTLLKALVRQQLPEKYKEIF
FDQSKNGYAGYIDGGASQEEFYKFIKPILEKMDGTEELLVKLNREDLLRK
QRTFDNGSIPHQIHLGELHAILRRQEDFYPFLKDNREKIEKILTFRIPYY
VGPLARGNSRFAWMTRKSEETITPWNFEEVVDKGASAQSFIERMTNFDKN
LPNEKVLPKHSLLYEYFTVYNELTKVKYVTEGMRKPAFLSGEQKKAIVDL
LFKTNRKVTVKQLKEDYFKKIECFDSVEISGVEDRFNASLGTYHDLLKII
KDKDFLDNEENEDILEDIVLTLTLFEDREMIEERLKTYAHLFDDKVMKQL
KRRRYTGWGRLSRKLINGIRDKQSGKTILDFLKSDGFANRNFMQLIHDDS
LTFKEDIQKAQVSGQGDSLHEHIANLAGSPAIKKGILQTVKVVDELVKVM
GRHKPENIVIEMARENQTTQKGQKNSRERMKRIEEGIKELGSQILKEHPV
ENTQLQNEKLYLYYLQNGRDMYVDQELDINRLSDYDVDHIVPQSFLKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEVQ
TGGFSKESILPKRNSDKLIARKKDWDPKKYGGF [symbol] SPTVAYSVLVVAKVEK
GKSKKLKSVKELLGITIMERSSFEKNPIDFLEAKGYKEVKKDLIIKLPKY
SLFELENGRKRMLASAGELQKGNELALPSKYVNFLYLASHYEKLKGSPED
NEQKQLFVEQHKHYLDEIIEQISEFSKRVILADANLDKVLSAYNKHRDKP
IREQAENIIHLFTLTNLGAPAAFKYFDTTIDRK [symbol] Y [symbol] STKEVLDATLIHQS
ITGLYETRIDLSQL

GGD. In this sequence, residues E1135, Q1335 and R1337, which can be mutated from D1135, R1335, and T1337 to yield a SpEQR Cas9, are underlined and in bold.

The amino acid sequence of an exemplary PAM-binding SpVQR Cas9 is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGF [symbol] SPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRK [symbol] Y [symbol] STKEVLDATLIHQ
SITGLYETRIDLSQ

LGGD. In this sequence, residues V1135, Q1335, and R1337, which can be mutated from D1135, R1335, and T1337 to yield a SpVQR Cas9, are underlined and in bold.

The amino acid sequence of an exemplary PAM-binding SpVRER Cas9 is as follows:

MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGF [symbol] SPTVAYSVLVVAKVE
KGKSKKLKSVKELLGITIMERSSFEKNPIDFLEAKGYKEVKKDLIIKLPK
YSLFELENGRKRMLASA [symbol] ELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRK [symbol] Y [symbol] STKEVLDATLIHQ
SITGLYETRIDLSQLGGD.

In the above sequence, residues V1135, R1218, Q1335, and R1337, which can be mutated from D1134, G1217, R1335, and T1337 to yield a SpVRER Cas9, are underlined and in bold.

In some embodiments, the Cas9 domain is a recombinant Cas9 domain. In some embodiments, the recombinant Cas9 domain is a SpyMacCas9 domain. In some embodiments, the SpyMacCas9 domain is a nuclease active SpyMacCas9, a nuclease inactive SpyMacCas9 (SpyMacCas9d), or a SpyMacCas9 nickase (SpyMacCas9n). In some embodiments, the SaCas9 domain, the SaCas9d domain, or the SaCas9n domain can bind to a nucleic acid sequence having a non-canonical PAM. In some embodiments, the SpyMacCas9 domain, the SpCas9d domain, or the SpCas9n domain can bind to a nucleic acid sequence having a NAA PAM sequence.

Exemplary SpyMacCas9
MDKKYSIGLDIGTNSVGWAVITDDYKVPSKKFKVLGNTDRHSIKKNLIGA
LLFGSGETAEATRLKRTARRRYTRRKNRICYLQEIFSNEMAKVDDSFFHR
LEESFLVEEDKKHERHPIFGNIVDEVAYHEKYPTIYHLRKKLADSTDKAD
LRLIYLALAHMIKFRGHFLIEGDLNPDNSDVDKLFIQLVQIYNQLFEENP
INASRVDAKAILSARLSKSRRLENLIAQLPGEKRNGLFGNLIALSLGLTP
NFKSNFDLAEDAKLQLSKDTYDDDLDNLLAQIGDQYADLFLAAKNLSDAI
LLSDILRVNSEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGAYHDLLKI
IKDKDFLDNEENEDILEDIVLTLTLFEDRGMIEERLKTYAHLFDDKVMKQ
LKRRRYTGWGRLSRKLINGIRDKQSGKTILDFLKSDGFANRNFMQLIHDD
SLTFKEDIQKAQVSGQGHSLHEQIANLAGSPAIKKGILQTVKIVDELVKV
MGHKPENIVIEMARENQTTQKGQKNSRERMKRIEEGIKELGSQILKEHPV
ENTQLQNEKLYLYYLQNGRDMYVDQELDINRLSDYDVDHIVPQSFIKDDS
IDNKVLTRSDKNRGKSDNVPSEEVVKKMKNYWRQLLNAKLITQRKFDNLT
KAERGGLSELDKAGFIKRQLVETRQITKHVAQILDSRMNTKYDENDKLIR
EVKVITLKSKLVSDFRKDFQFYKVREINNYHHAHDAYLNAVVGTALIKKY
PKLESEFVYGDYKVYDVRKMIAKSEQEIGKATAKYFFYSNIMNFFKTEIT
LANGEIRKRPLIETNGETGEIVWDKGRDFATVRKVLSMPQVNIVKKTEIQ
TVGQNGGLFDDNPKSPLEVITSKLVPLKKELNPKKYGGYQKPITAYPVLL
ITDTKQLIPISVMNKKQFEQNPVKFLRDRGYQQVGKNDFIKLPKYTLVDI
GDGIKRLWASSKEIHKGNQLVVSKKSQILLYHAHHLDSDLSNDYLQNHNQ
QFDVLFNEIISFSKKCKLGKEHIQKIENVYSNKKNSASIEELAESFIKLL
GFTQLGATSPFNFLGVKLNQKQYKGKKDYILPCTEGTLIRQSITGLYETR
VDLSKIGED.

In some cases, a variant Cas9 protein harbors, H840A, P475A, W476A, N477A, D1125A, W1126A, and D1218A mutations such that the polypeptide has a reduced ability to cleave a target DNA or RNA. Such a Cas9 protein has a reduced ability to cleave a target DNA (e.g., a single stranded target DNA) but retains the ability to bind a target DNA (e.g., a single stranded target DNA). As another non-limiting example, in some cases, the variant Cas9 protein harbors D10A, H840A, P475A, W476A, N477A, D1125A, W1126A, and D1218A mutations such that the polypeptide has a reduced ability to cleave a target DNA. Such a Cas9 protein has a reduced ability to cleave a target DNA (e.g., a single stranded target DNA) but retains the ability to bind a target DNA (e.g., a single stranded target DNA). In some cases, when a variant Cas9 protein harbors W476A and W1126A mutations or when the variant Cas9 protein harbors P475A, W476A, N477A, D1125A, W1126A, and D1218A mutations, the variant Cas9 protein does not bind efficiently to a PAM sequence. Thus, in some such cases, when such a variant Cas9 protein is used in a method of binding, the method does not require a PAM sequence. In other words, in some cases, when such a variant Cas9 protein is used in a method of binding, the method can include a guide RNA, but the method can be performed in the absence of a PAM sequence (and the specificity of binding is therefore provided by the targeting segment of the guide RNA). Other residues can be mutated to achieve the above effects (i.e., inactivate one or the other nuclease portions). As non-limiting examples, residues D10, G12, G17, E762, H840, N854, N863, H982, H983, A984, D986, and/or A987 can be altered (i.e., substituted). Also, mutations other than alanine substitutions are suitable.

In some embodiments, a CRISPR protein-derived domain of a base editor can comprise all or a portion of a Cas9 protein with a canonical PAM sequence (NGG). In other embodiments, a Cas9-derived domain of a base editor can employ a non-canonical PAM sequence. Such sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

Cas9 Domains with Reduced Exclusivity

Typically, Cas9 proteins, such as Cas9 from S. pyogenes (spCas9), require a canonical NGG PAM sequence to bind a particular nucleic acid region, where the “N” in “NGG” is adenosine (A), thymidine (T), or cytosine (C), and the G is guanosine. This may limit the ability to edit desired bases within a genome. In some embodiments, the base editing fusion proteins provided herein may need to be placed at a precise location, for example a region comprising a target base that is upstream of the PAM. See e.g., Komor, A. C., et al., “Programmable editing of a target base in genomic DNA without double-stranded DNA cleavage” Nature 533, 420-424 (2016), the entire contents of which are hereby incorporated by reference. Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); Nishimasu, H., et al., “Engineered CRISPR-Cas9 nuclease with expanded targeting space” Science. 2018 Sep. 21; 361(6408):1259-1262, Chatterjee, P., et al., Minimal PAM specificity of a highly similar SpCas9 ortholog” Sci Adv. 2018 Oct. 24; 4(10):eaau0766. doi: 10.1126/sciadv.aau0766, the entire contents of each are hereby incorporated by reference.

Fusion Proteins Comprising a Cas9 Domain and a Cytidine Deaminase and/or Adenosine Deaminase

Some aspects of the disclosure provide fusion proteins comprising a Cas9 domain or other nucleic acid programmable DNA binding protein and one or more adenosine deaminase domain, cytidine deaminase domain, and/or DNA glycosylase domains. It should be appreciated that the Cas9 domain may be any of the Cas9 domains or Cas9 proteins (e.g., dCas9 or nCas9) provided herein. In some embodiments, any of the Cas9 domains or Cas9 proteins (e.g., dCas9 or nCas9) provided herein may be fused with any of the cytidine deaminases and adenosine deaminases provided herein. The domains of the base editors disclosed herein can be arranged in any order.

For example, and without limitation, in some embodiments, the fusion protein comprises the structure:

NH₂-[cytidine deaminase]-[Cas9 domain]-[adenosine deaminase]-COOH;
NH₂-[adenosine deaminase]-[Cas9 domain]-[cytidine deaminase]-COOH;
NH₂-[adenosine deaminase]-[cytidine deaminase]-[Cas9 domain]-COOH;
NH₂-[cytidine deaminase]-[adenosine deaminase]-[Cas9 domain]-COOH;
NH₂-[Cas9 domain]-[adenosine deaminase]-[cytidine deaminase]-COOH; or
NH₂-[Cas9 domain]-[cytidine deaminase]-[adenosine deaminase]-COOH.

In some embodiments, the adenosine deaminase of the fusion protein comprises a TadA*8 and a cytidine deaminase. In some embodiments, the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

Exemplary fusion protein structures include the following:

NH₂-[adenosine deaminase]-[Cas9]-[cytidine deaminase]-COOH;
NH₂-[cytidine deaminase]-[Cas9]-[adenosine deaminase]-COOH;
NH₂-[TadA*8]-[Cas9]-[cytidine deaminase]-COOH; or
NH₂-[cytidine deaminase]-[Cas9]-[TadA*8]-COOH.

In some embodiments, the fusion proteins comprising a cytidine deaminase, abasic editor, and adenosine deaminase and a napDNAbp (e.g., Cas9 domain) do not include a linker sequence. In some embodiments, a linker is present between the cytidine deaminase and adenosine deaminase domains and the napDNAbp. In some embodiments, the “-” used in the general architecture above indicates the presence of an optional linker. In some embodiments, the cytidine deaminase and adenosine deaminase and the napDNAbp are fused via any of the linkers provided herein. For example, in some embodiments the cytidine deaminase and adenosine deaminase and the napDNAbp are fused via any of the linkers provided below in the section entitled “Linkers”.

In some embodiments, the general architecture of exemplary Cas9 or Cas12 fusion proteins with a cytidine deaminase, adenosine deaminase and a Cas9 or Cas12 domain comprises any one of the following structures, where NLS is a nuclear localization sequence (e.g., any NLS provided herein), NH₂ is the N-terminus of the fusion protein, and COOH is the C-terminus of the fusion protein.

NH₂-NLS-[cytidine deaminase]-[Cas9 domain]-[adenosine deaminase]-COOH;

NH₂-NLS-[adenosine deaminase]-[Cas9 domain]-[cytidine deaminase]-COOH;

NH₂-NLS-[adenosine deaminase] [cytidine deaminase]-[Cas9 domain]-COOH;

NH₂-NLS-[cytidine deaminase]-[adenosine deaminase]-[Cas9 domain]-COOH;

NH₂-NLS-[Cas9 domain]-[adenosine deaminase]-[cytidine deaminase]-COOH;

NH₂-NLS-[Cas9 domain]-[cytidine deaminase]-[adenosine deaminase]-COOH;

NH₂-[cytidine deaminase]-[Cas9 domain]-[adenosine deaminase]-NLS—COOH;

NH₂-[adenosine deaminase]-[Cas9 domain]-[cytidine deaminase]-NL2-COOH;

NH₂-[adenosine deaminase] [cytidine deaminase]-[Cas9 domain]-NLS—COOH;

NH₂-[cytidine deaminase]-[adenosine deaminase]-[Cas9 domain]-NLS—COOH;

NH₂-[Cas9 domain]-[adenosine deaminase]-[cytidine deaminase]-NLS—COOH; or

NH₂-[Cas9 domain]-[cytidine deaminase]-[adenosine deaminase]-NLS—COOH.

In some embodiments, the NLS is present in a linker or the NLS is flanked by linkers, for example described herein. In some embodiments, the N-terminus or C-terminus NLS is a bipartite NLS. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK, is the prototype of the ubiquitous bipartite signal: two clusters of basic amino acids, separated by a spacer of about 10 amino acids. The sequence of an exemplary bipartite NLS follows: PKKKRKVEGADKRTADGSEFESPKKKRKV.

In some embodiments, the fusion proteins comprising a cytidine deaminase, adenosine deaminase, a Cas9 domain and an NLS do not comprise a linker sequence. In some embodiments, linker sequences between one or more of the domains or proteins (e.g., cytidine deaminase, adenosine deaminase, Cas9 domain or NLS) are present.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Exemplary, yet nonlimiting, fusion proteins are described in International PCT Application Nos. PCT/2017/044935 and PCT/US2020/016288, each of which is incorporated herein by reference for its entirety.

### Fusion Proteins Comprising a Nuclear Localization Sequence (NLS)

In some embodiments, the fusion proteins provided herein further comprise one or more (e.g., 2, 3, 4, 5) nuclear targeting sequences, for example a nuclear localization sequence (NLS). In one embodiment, a bipartite NLS is used. In some embodiments, a NLS comprises an amino acid sequence that facilitates the importation of a protein, that comprises an NLS, into the cell nucleus (e.g., by nuclear transport). In some embodiments, any of the fusion proteins provided herein further comprise a nuclear localization sequence (NLS). In some embodiments, the NLS is fused to the N-terminus of the fusion protein. In some embodiments, the NLS is fused to the C-terminus of the fusion protein. In some embodiments, the NLS is fused to the N-terminus of the Cas9 domain. In some embodiments, the NLS is fused to the C-terminus of an nCas9 domain or a dCas9 domain. In some embodiments, the NLS is fused to the N-terminus of the deaminase. In some embodiments, the NLS is fused to the C-terminus of the deaminase. In some embodiments, the NLS is fused to the fusion protein via one or more linkers. In some embodiments, the NLS is fused to the fusion protein without a linker. In some embodiments, the NLS comprises an amino acid sequence of any one of the NLS sequences provided or referenced herein. Additional nuclear localization sequences are known in the art and would be apparent to the skilled artisan. For example, NLS sequences are described in Plank et al., PCT/EP2000/011690, the contents of which are incorporated herein by reference for their disclosure of exemplary nuclear localization sequences. In some embodiments, an NLS comprises the amino acid sequence PKKKRKVEGADKRTADGSE FESPKKKRKV, KRTADGSEFESPKKKRKV, KRPAATKKAGQAKKKK, KKTELQTTNAENKTKKL, KRGINDRNFWRGENGRKTR, RKSGKIAAIVVKRPRKPKKKRKV, or MDSLLMNRRKFLYQFKNVRWAKGRRETYLC.

In some embodiments, the NLS is present in a linker or the NLS is flanked by linkers, for example, the linkers described herein. In some embodiments, the N-terminus or C-terminus NLS is a bipartite NLS. A bipartite NLS comprises two basic amino acid clusters, which are separated by a relatively short spacer sequence (hence bipartite—2 parts, while monopartite NLSs are not). The NLS of nucleoplasmin, KR[PAATKKAGQA]KKKK, is the prototype of the ubiquitous bipartite signal: two clusters of basic amino acids, separated by a spacer of about 10 amino acids. The sequence of an exemplary bipartite NLS follows:

PKKKRKVEGADKRTADGSEFESPKKKRKV

In some embodiments, the fusion proteins do not comprise a linker sequence. In some embodiments, linker sequences between one or more of the domains or proteins are present. In some embodiments, the general architecture of exemplary Cas9 fusion proteins with an adenosine deaminase or a cytidine deaminase and a Cas9 domain comprises any one of the following structures, where NLS is a nuclear localization sequence (e.g., any NLS provided herein), NH₂ is the N-terminus of the fusion protein, and COOH is the C-terminus of the fusion protein:

NH₂-NLS-[adenosine deaminase]-[Cas9 domain]-COOH;

NH₂-NLS [Cas9 domain]-[adenosine deaminase]-COOH;

NH₂-[adenosine deaminase]-[Cas9 domain]-NLS—COOH;

NH₂-[Cas9 domain]-[adenosine deaminase]-NLS—COOH;

NH₂-NLS-[cytidine deaminase]-[Cas9 domain]-COOH;

NH₂-NLS [Cas9 domain]-[cytidine deaminase]-COOH;

NH₂-[cytidine deaminase]-[Cas9 domain]-NLS—COOH; or

NH₂-[Cas9 domain]-[cytidine deaminase]-NLS—COOH.

It should be appreciated that the fusion proteins of the present disclosure may comprise one or more additional features. For example, in some embodiments, the fusion protein may comprise inhibitors, cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

A vector that encodes a CRISPR enzyme comprising one or more nuclear localization sequences (NLSs) can be used. For example, there can be or be about 1, 2, 3, 4, 5, 6, 7, 8, 9, 10 NLSs used. A CRISPR enzyme can comprise the NLSs at or near the ammo-terminus, about or more than about 1, 2, 3, 4, 5, 6, 7, 8, 9, 10 NLSs at or near the carboxy-terminus, or any combination of these (e.g., one or more NLS at the ammo-terminus and one or more NLS at the carboxy terminus). When more than one NLS is present, each can be selected independently of others, such that a single NLS can be present in more than one copy and/or in combination with one or more other NLSs present in one or more copies.

CRISPR enzymes used in the methods can comprise about 6 NLSs. An NLS is considered near the N- or C-terminus when the nearest amino acid to the NLS is within about 50 amino acids along a polypeptide chain from the N- or C-terminus, e.g., within 1, 2, 3, 4, 5, 10, 15, 20, 25, 30, 40, or 50 amino acids.

Fusion Proteins with Internal Insertions

Provided herein are fusion proteins comprising a heterologous polypeptide fused to a nucleic acid programmable nucleic acid binding protein, for example, a napDNAbp. A heterologous polypeptide can be a polypeptide that is not found in the native or wild-type napDNAbp polypeptide sequence. The heterologous polypeptide can be fused to the napDNAbp at a C-terminal end of the napDNAbp, an N-terminal end of the napDNAbp, or inserted at an internal location of the napDNAbp. In some embodiments, the heterologous polypeptide is inserted at an internal location of the napDNAbp.

In some embodiments, the heterologous polypeptide is a deaminase or a functional fragment thereof. For example, a fusion protein can comprise a deaminase flanked by an N-terminal fragment and a C-terminal fragment of a Cas9 or Cas12 (e.g., Cas12b/C2c1), polypeptide. The deaminase in a fusion protein can be an adenosine deaminase. In some embodiments, the adenosine deaminase is a TadA (e.g., TadA7.10 or TadA*8). In some embodiments, the TadA is a TadA*8. TadA sequences (e.g., TadA7.10 or TadA*8) as described herein are suitable deaminases for the above-described fusion proteins.

The deaminase can be a circular permutant deaminase. For example, the deaminase can be a circular permutant adenosine deaminase. In some embodiments, the deaminase is a circular permutant TadA, circularly permutated at amino acid residue 116 as numbered in the TadA reference sequence. In some embodiments, the deaminase is a circular permutant TadA, circularly permutated at amino acid residue 136 as numbered in the TadA reference sequence. In some embodiments, the deaminase is a circular permutant TadA, circularly permutated at amino acid residue 65 as numbered in the TadA reference sequence.

The fusion protein can comprise more than one deaminase. The fusion protein can comprise, for example, 1, 2, 3, 4, 5 or more deaminases. In some embodiments, the fusion protein comprises one deaminase. In some embodiments, the fusion protein comprises two deaminases. The two or more deaminases in a fusion protein can be an adenosine deaminase. cytidine deaminase, or a combination thereof. The two or more deaminases can be homodimers. The two or more deaminases can be heterodimers. The two or more deaminases can be inserted in tandem in the napDNAbp. In some embodiments, the two or more deaminases may not be in tandem in the napDNAbp.

In some embodiments, the napDNAbp in the fusion protein is a Cas9 polypeptide or a fragment thereof. The Cas9 polypeptide can be a variant Cas9 polypeptide. In some embodiments, the Cas9 polypeptide is a Cas9 nickase (nCas9) polypeptide or a fragment thereof. In some embodiments, the Cas9 polypeptide is a nuclease dead Cas9 (dCas9) polypeptide or a fragment thereof. The Cas9 polypeptide in a fusion protein can be a full-length Cas9 polypeptide. In some cases, the Cas9 polypeptide in a fusion protein may not be a full length Cas9 polypeptide. The Cas9 polypeptide can be truncated, for example, at a N-terminal or C-terminal end relative to a naturally-occurring Cas9 protein. The Cas9 polypeptide can be a circularly permuted Cas9 protein. The Cas9 polypeptide can be a fragment, a portion, or a domain of a Cas9 polypeptide, that is still capable of binding the target polynucleotide and a guide nucleic acid sequence.

In some embodiments, the Cas9 polypeptide is a Streptococcus pyogenes Cas9 (SpCas9), Staphylococcus aureus Cas9 (SaCas9), Streptococcus thermophilus I Cas9 (St1Cas9), or fragments or variants thereof.

The Cas9 polypeptide of a fusion protein can comprise an amino acid sequence that is at least 85%, at least 90%, at least 91%, at least 92%, at least 93%, at least 94%, at least 95%, at least 96%, at least 97%, at least 98%, at least 99%, or at least 99.5% identical to a naturally-occurring Cas9 polypeptide.

The Cas9 polypeptide of a fusion protein can comprise an amino acid sequence that is at least 85%, at least 90%, at least 91%, at least 92%, at least 93%, at least 94%, at least 95%, at least 96%, at least 97%, at least 98%, at least 99%, or at least 99.5% identical to the Cas9 amino acid sequence set forth below (called the “Cas9 reference sequence” below):

MDKKYSIGLDIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD
(single underline: HNH domain; double underline:
RuvC domain).

Fusion proteins comprising a heterologous catalytic domain flanked by N- and C-terminal fragments of a Cas9 polypeptide are also useful for base editing in the methods as described herein. Fusion proteins comprising Cas9 and one or more deaminase domains, e.g., adenosine deaminase, or comprising an adenosine deaminase domain flanked by Cas9 sequences are also useful for highly specific and efficient base editing of target sequences. In an embodiment, a chimeric Cas9 fusion protein contains a heterologous catalytic domain (e.g., adenosine deaminase, cytidine deaminase, or adenosine deaminase and cytidine deaminase) inserted within a Cas9 polypeptide. In some embodiments, the fusion protein comprises an adenosine deaminase domain and a cytidine deaminase domain inserted within a Cas9. In some embodiments, an adenosine deaminase is fused within a Cas9 and a cytidine deaminase is fused to the C-terminus. In some embodiments, an adenosine deaminase is fused within Cas9 and a cytidine deaminase fused to the N-terminus. In some embodiments, a cytidine deaminase is fused within Cas9 and an adenosine deaminase is fused to the C-terminus. In some embodiments, a cytidine deaminase is fused within Cas9 and an adenosine deaminase fused to the N-terminus.

Exemplary structures of a fusion protein with an adenosine deaminase and a cytidine deaminase and a Cas9 are provided as follows:

NH₂-[Cas9(adenosine deaminase)]-[cytidine deaminase]-COOH;

NH₂-[cytidine deaminase]-[Cas9(adenosine deaminase)]-COOH;

NH₂-[Cas9(cytidine deaminase)]-[adenosine deaminase]-COOH; or

NH₂-[adenosine deaminase]-[Cas9(cytidine deaminase)]-COOH.

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA7.10). In some embodiments, the TadA is a TadA*8. In some embodiments, a TadA*8 is fused within Cas9 and a cytidine deaminase is fused to the C-terminus. In some embodiments, a TadA*8 is fused within Cas9 and a cytidine deaminase fused to the N-terminus. In some embodiments, a cytidine deaminase is fused within Cas9 and a TadA*8 is fused to the C-terminus. In some embodiments, a cytidine deaminase is fused within Cas9 and a TadA*8 fused to the N-terminus. Exemplary structures of a fusion protein with a TadA*8 and a cytidine deaminase and a Cas9 are provided as follows:

NH₂-[Cas9(TadA*8)]-[cytidine deaminase]-COOH;

NH₂-[cytidine deaminase]-[Cas9(TadA*8)]-COOH;

NH₂-[Cas9(cytidine deaminase)]-[TadA*8]-COOH; or

NH₂-[TadA*8]-[Cas9(cytidine deaminase)]-COOH.

In some embodiments, the “-” used in the general architecture above indicates the presence of an optional linker.

The heterologous polypeptide (e.g., deaminase) can be inserted in the napDNAbp (e.g., Cas9 or Cas12 (e.g., Cas12b/C2c1)) at a suitable location, for example, such that the napDNAbp retains its ability to bind the target polynucleotide and a guide nucleic acid. A deaminase (e.g., adenosine deaminase, cytidine deaminase, or adenosine deaminase and cytidine deaminase) can be inserted into a napDNAbp without compromising function of the deaminase (e.g., base editing activity) or the napDNAbp (e.g., ability to bind to target nucleic acid and guide nucleic acid). A deaminase (e.g., adenosine deaminase, cytidine deaminase, or adenosine deaminase and cytidine deaminase) can be inserted in the napDNAbp at, for example, a disordered region or a region comprising a high temperature factor or B-factor as shown by crystallographic studies. Regions of a protein that are less ordered, disordered, or unstructured, for example solvent exposed regions and loops, can be used for insertion without compromising structure or function. A deaminase (e.g., adenosine deaminase, cytidine deaminase, or adenosine deaminase and cytidine deaminase) can be inserted in the napDNAbp in a flexible loop region or a solvent-exposed region. In some embodiments, the deaminase (e.g., adenosine deaminase, cytidine deaminase, or adenosine deaminase and cytidine deaminase) is inserted in a flexible loop of the Cas9 or the Cas12b/C2c1 polypeptide.

In some embodiments, the insertion location of a deaminase (e.g., adenosine deaminase, cytidine deaminase, or adenosine deaminase and cytidine deaminase) is determined by B-factor analysis of the crystal structure of Cas9 polypeptide. In some embodiments, the deaminase (e.g., adenosine deaminase, cytidine deaminase, or adenosine deaminase and cytidine deaminase) is inserted in regions of the Cas9 polypeptide comprising higher than average B-factors (e.g., higher B factors compared to the total protein or the protein domain comprising the disordered region). B-factor or temperature factor can indicate the fluctuation of atoms from their average position (for example, as a result of temperature-dependent atomic vibrations or static disorder in a crystal lattice). A high B-factor (e.g., higher than average B-factor) for backbone atoms can be indicative of a region with relatively high local mobility. Such a region can be used for inserting a deaminase without compromising structure or function. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200%, or greater than 200% more than the average B-factor for the total protein. A deaminase (e.g., adenosine deaminase, cytidine deaminase, or adenosine deaminase and cytidine deaminase) can be inserted at a location with a residue having a Ca atom with a B-factor that is 50%, 60%, 70%, 80%, 90%, 100%, 110%, 120%, 130%, 140%, 150%, 160%, 170%, 180%, 190%, 200% or greater than 200% more than the average B-factor for a Cas9 protein domain comprising the residue. Cas9 polypeptide positions comprising a higher than average B-factor can include, for example, residues 768, 792, 1052, 1015, 1022, 1026, 1029, 1067, 1040, 1054, 1068, 1246, 1247, and 1248 as numbered in the above Cas9 reference sequence. Cas9 polypeptide regions comprising a higher than average B-factor can include, for example, residues 792-872, 792-906, and 2-791 as numbered in the above Cas9 reference sequence.

A heterologous polypeptide (e.g., deaminase) can be inserted in the napDNAbp at an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. In some embodiments, the heterologous polypeptide is inserted between amino acid positions 768-769, 791-792, 792-793, 1015-1016, 1022-1023, 1026-1027, 1029-1030, 1040-1041, 1052-1053, 1054-1055, 1067-1068, 1068-1069, 1247-1248, or 1248-1249 as numbered in the above Cas9 reference sequence or corresponding amino acid positions thereof. In some embodiments, the heterologous polypeptide is inserted between amino acid positions 769-770, 792-793, 793-794, 1016-1017, 1023-1024, 1027-1028, 1030-1031, 1041-1042, 1053-1054, 1055-1056, 1068-1069, 1069-1070, 1248-1249, or 1249-1250 as numbered in the above Cas9 reference sequence or corresponding amino acid positions thereof. In some embodiments, the heterologous polypeptide replaces an amino acid residue selected from the group consisting of: 768, 791, 792, 1015, 1016, 1022, 1023, 1026, 1029, 1040, 1052, 1054, 1067, 1068, 1069, 1246, 1247, and 1248 as numbered in the above Cas9 reference sequence, or a corresponding amino acid residue in another Cas9 polypeptide. It should be understood that the reference to the above Cas9 reference sequence with respect to insertion positions is for illustrative purposes. The insertions as discussed herein are not limited to the Cas9 polypeptide sequence of the above Cas9 reference sequence, but include insertion at corresponding locations in variant Cas9 polypeptides, for example a Cas9 nickase (nCas9), nuclease dead Cas9 (dCas9), a Cas9 variant lacking a nuclease domain, a truncated Cas9, or a Cas9 domain lacking partial or complete HNH domain.

A heterologous polypeptide (e.g., deaminase) can be inserted in the napDNAbp at an amino acid residue selected from the group consisting of: 768, 792, 1022, 1026, 1040, 1068, and 1247 as numbered in the above Cas9 reference sequence, or a corresponding amino acid residue in another Cas9 polypeptide. In some embodiments, the heterologous polypeptide is inserted between amino acid positions 768-769, 792-793, 1022-1023, 1026-1027, 1029-1030, 1040-1041, 1068-1069, or 1247-1248 as numbered in the above Cas9 reference sequence or corresponding amino acid positions thereof. In some embodiments, the heterologous polypeptide is inserted between amino acid positions 769-770, 793-794, 1023-1024, 1027-1028, 1030-1031, 1041-1042, 1069-1070, or 1248-1249 as numbered in the above Cas9 reference sequence or corresponding amino acid positions thereof. In some embodiments, the heterologous polypeptide replaces an amino acid residue selected from the group consisting of: 768, 792, 1022, 1026, 1040, 1068, and 1247 as numbered in the above Cas9 reference sequence, or a corresponding amino acid residue in another Cas9 polypeptide.

A heterologous polypeptide (e.g., deaminase) can be inserted in the napDNAbp at an amino acid residue as described herein, or a corresponding amino acid residue in another Cas9 polypeptide. In an embodiment, a heterologous polypeptide (e.g., deaminase) can be inserted in the napDNAbp at an amino acid residue selected from the group consisting of: 1002, 1003, 1025, 1052-1056, 1242-1247, 1061-1077, 943-947, 686-691, 569-578, 530-539, and 1060-1077 as numbered in the above Cas9 reference sequence, or a corresponding amino acid residue in another Cas9 polypeptide. The deaminase (e.g., adenosine deaminase, cytidine deaminase, or adenosine deaminase and cytidine deaminase) can be inserted at the N-terminus or the C-terminus of the residue or replace the residue. In some embodiments, the deaminase (e.g., adenosine deaminase, cytidine deaminase, or adenosine deaminase and cytidine deaminase) is inserted at the C-terminus of the residue.

In some embodiments, an adenosine deaminase (e.g., TadA) is inserted at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, an adenosine deaminase (e.g., TadA) is inserted in place of residues 792-872, 792-906, or 2-791 as numbered in the above Cas9 reference sequence, or a corresponding amino acid residue in another Cas9 polypeptide. In some embodiments, the adenosine deaminase is inserted at the N-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted at the C-terminus of an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide. In some embodiments, the adenosine deaminase is inserted to replace an amino acid selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

In some embodiments, a CBE (e.g., APOBEC1) is inserted at an amino acid residue selected from the group consisting of: 1016, 1023, 1029, 1040, 1069, and 1247 as numbered in the above Cas9 reference sequence, or a corresponding amino acid residue in another Cas9 polypeptide. In some embodiments, the ABE is inserted at the N-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the ABE is inserted at the C-terminus of an amino acid selected from the group consisting of: 1016, 1023, 1029, 1040, 1069, and 1247 as numbered in the above Cas9 reference sequence, or a corresponding amino acid residue in another Cas9 polypeptide. In some embodiments, the ABE is inserted to replace an amino acid selected from the group consisting of: 1016, 1023, 1029, 1040, 1069, and 1247 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 768 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 768 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 791 or is inserted at amino acid residue 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid 791 or is inserted at the N-terminus of amino acid 792, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid 791, or is inserted to replace amino acid 792, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1016 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1016 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1022, or is inserted at amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1022 or is inserted at the N-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1022 or is inserted at the C-terminus of amino acid residue 1023, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1022, or is inserted to replace amino acid residue 1023, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1026, or is inserted at amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1026 or is inserted at the N-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1026 or is inserted at the C-terminus of amino acid residue 1029, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1026, or is inserted to replace amino acid residue 1029, as numbered in the above Cas9 reference sequence, or corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1040 as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1040 as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1052, or is inserted at amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1052 or is inserted at the N-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1052 or is inserted at the C-terminus of amino acid residue 1054, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1052, or is inserted to replace amino acid residue 1054, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1067, or is inserted at amino acid residue 1068, or is inserted at amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1067 or is inserted at the N-terminus of amino acid residue 1068 or is inserted at the N-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1067 or is inserted at the C-terminus of amino acid residue 1068 or is inserted at the C-terminus of amino acid residue 1069, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1067, or is inserted to replace amino acid residue 1068, or is inserted to replace amino acid residue 1069, as numbered in the above Cas9 reference sequence, or a corresponding amino acid residue in another Cas9 polypeptide.

In some embodiments, the deaminase (e.g., adenosine deaminase, cytidine deaminase, or adenosine deaminase and cytidine deaminase) is inserted at amino acid residue 1246, or is inserted at amino acid residue 1247, or is inserted at amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the N-terminus of amino acid residue 1246 or is inserted at the N-terminus of amino acid residue 1247 or is inserted at the N-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted at the C-terminus of amino acid residue 1246 or is inserted at the C-terminus of amino acid residue 1247 or is inserted at the C-terminus of amino acid residue 1248, as numbered in the above Cas9 reference sequence, or a corresponding amino acid residue in another Cas9 polypeptide. In some embodiments, the deaminase (e.g., adenosine deaminase, cytidine deaminase, or adenosine deaminase and cytidine deaminase) is inserted to replace amino acid residue 1246, or is inserted to replace amino acid residue 1247, or is inserted to replace amino acid residue 1248, as numbered in the above Cas9 reference sequence, or a corresponding amino acid residue in another Cas9 polypeptide.

In some embodiments, a heterologous polypeptide (e.g., deaminase) is inserted in a flexible loop of a Cas9 polypeptide. The flexible loop portions can be selected from the group consisting of 530-537, 569-570, 686-691, 943-947, 1002-1025, 1052-1077, 1232-1247, or 1298-1300 as numbered in the above Cas9 reference sequence, or a corresponding amino acid residue in another Cas9 polypeptide. The flexible loop portions can be selected from the group consisting of: 1-529, 538-568, 580-685, 692-942, 948-1001, 1026-1051, 1078-1231, or 1248-1297 as numbered in the above Cas9 reference sequence, or a corresponding amino acid residue in another Cas9 polypeptide.

A heterologous polypeptide (e.g., adenine deaminase) can be inserted into a Cas9 polypeptide region corresponding to amino acid residues: 1017-1069, 1242-1247, 1052-1056, 1060-1077, 1002-1003, 943-947, 530-537, 568-579, 686-691, 1242-1247, 1298-1300, 1066-1077, 1052-1056, or 1060-1077 as numbered in the above Cas9 reference sequence, or a corresponding amino acid residue in another Cas9 polypeptide.

A heterologous polypeptide (e.g., adenine deaminase) can be inserted in place of a deleted region of a Cas9 polypeptide. The deleted region can correspond to an N-terminal or C-terminal portion of the Cas9 polypeptide. In some embodiments, the deleted region corresponds to residues 792-872 as numbered in the above Cas9 reference sequence, or a corresponding amino acid residue in another Cas9 polypeptide. In some embodiments, the deleted region corresponds to residues 792-906 as numbered in the above Cas9 reference sequence, or a corresponding amino acid residue in another Cas9 polypeptide. In some embodiments, the deleted region corresponds to residues 2-791 as numbered in the above Cas9 reference sequence, or a corresponding amino acid residue in another Cas9 polypeptide. In some embodiments, the deleted region corresponds to residues 1017-1069 as numbered in the above Cas9 reference sequence, or corresponding amino acid residues thereof.

Exemplary internal fusions base editors are provided in Table 5A below:

TABLE 5A
Insertion loci in Cas9 proteins

| BE ID | Modification | Other ID |
|---|---|---|
| IBE001 | Cas9 TadA ins 1015 | ISLAY01 |
| IBE002 | Cas9 TadA ins 1022 | ISLAY02 |
| IBE003 | Cas9 TadA ins 1029 | ISLAY03 |
| IBE004 | Cas9 TadA ins 1040 | ISLAY04 |
| IBE005 | Cas9 TadA ins 1068 | ISLAY05 |
| IBE006 | Cas9 TadA ins 1247 | ISLAY06 |
| IBE007 | Cas9 TadA ins 1054 | ISLAY07 |
| IBE008 | Cas9 TadA ins 1026 | ISLAY08 |
| IBE009 | Cas9 TadA ins 768 | ISLAY09 |
| IBE020 | delta HNH TadA 792 | ISLAY20 |
| IBE021 | N-term fusion single TadA helix | ISLAY21 |
| | truncated 165-end | |
| IBE029 | TadA-Circular Permutant116 ins1067 | ISLAY29 |
| IBE031 | TadA- Circular Permutant 136 ins1248 | ISLAY31 |
| IBE032 | TadA- Circular Permutant 136ins 1052 | ISLAY32 |
| IBE035 | delta 792-872 TadA ins | I LAY35 |
| IBE036 | delta 792-906 TadA ins | ISLAY36 |
| IBE043 | TadA-Circular Permutant 65 ins1246 | ISLAY43 |
| IBE044 | TadA ins C-term truncate2 791 | ISLAY44 |

A heterologous polypeptide (e.g., deaminase) can be inserted within a structural or functional domain of a Cas9 polypeptide. A heterologous polypeptide (e.g., deaminase) can be inserted between two structural or functional domains of a Cas9 polypeptide. A heterologous polypeptide (e.g., deaminase) can be inserted in place of a structural or functional domain of a Cas9 polypeptide, for example, after deleting the domain from the Cas9 polypeptide. The structural or functional domains of a Cas9 polypeptide can include, for example, RuvC I, RuvC II, RuvC III, Rec1, Rec2, PI, or HNH.

In some embodiments, the Cas9 polypeptide lacks one or more domains selected from the group consisting of: RuvC I, RuvC II, RuvC III, Rec1, Rec2, PI, or HNH domain. In some embodiments, the Cas9 polypeptide lacks a nuclease domain. In some embodiments, the Cas9 polypeptide lacks an HNH domain. In some embodiments, the Cas9 polypeptide lacks a portion of the HNH domain such that the Cas9 polypeptide has reduced or abolished HNH activity. In some embodiments, the Cas9 polypeptide comprises a deletion of the nuclease domain, and the deaminase is inserted to replace the nuclease domain. In some embodiments, the HNH domain is deleted and the deaminase is inserted in its place. In some embodiments, one or more of the RuvC domains is deleted and the deaminase is inserted in its place.

A fusion protein comprising a heterologous polypeptide can be flanked by a N-terminal and a C-terminal fragment of a napDNAbp. In some embodiments, the fusion protein comprises a deaminase flanked by a N-terminal fragment and a C-terminal fragment of a Cas9 polypeptide. The N terminal fragment or the C terminal fragment can bind the target polynucleotide sequence. The C-terminus of the N terminal fragment or the N-terminus of the C terminal fragment can comprise a part of a flexible loop of a Cas9 polypeptide. The C-terminus of the N terminal fragment or the N-terminus of the C terminal fragment can comprise a part of an alpha-helix structure of the Cas9 polypeptide. The N-terminal fragment or the C-terminal fragment can comprise a DNA binding domain. The N-terminal fragment or the C-terminal fragment can comprise a RuvC domain. The N-terminal fragment or the C-terminal fragment can comprise an HNH domain. In some embodiments, neither of the N-terminal fragment and the C-terminal fragment comprises an HNH domain.

In some embodiments, the C-terminus of the N terminal Cas9 fragment comprises an amino acid that is in proximity to a target nucleobase when the fusion protein deaminates the target nucleobase. In some embodiments, the N-terminus of the C terminal Cas9 fragment comprises an amino acid that is in proximity to a target nucleobase when the fusion protein deaminates the target nucleobase. The insertion location of different deaminases can be different in order to have proximity between the target nucleobase and an amino acid in the C-terminus of the N terminal Cas9 fragment or the N-terminus of the C terminal Cas9 fragment. For example, the insertion position of an ABE can be at an amino acid residue selected from the group consisting of: 1015, 1022, 1029, 1040, 1068, 1247, 1054, 1026, 768, 1067, 1248, 1052, and 1246 as numbered in the above Cas9 reference sequence, or a corresponding amino acid residue in another Cas9 polypeptide.

The N-terminal Cas9 fragment of a fusion protein (i.e. the N-terminal Cas9 fragment flanking the deaminase in a fusion protein) can comprise the N-terminus of a Cas9 polypeptide. The N-terminal Cas9 fragment of a fusion protein can comprise a length of at least about: 100, 200, 300, 400, 500, 600, 700, 800, 900, 1000, 1100, 1200, or 1300 amino acids. The N-terminal Cas9 fragment of a fusion protein can comprise a sequence corresponding to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1-56, 1-95, 1-200, 1-300, 1-400, 1-500, 1-600, 1-700, 1-718, 1-765, 1-780, 1-906, 1-918, or 1-1100 as numbered in the above Cas9 reference sequence, or a corresponding amino acid residue in another Cas9 polypeptide.

The C-terminal Cas9 fragment of a fusion protein (i.e. the C-terminal Cas9 fragment flanking the deaminase in a fusion protein) can comprise the C-terminus of a Cas9 polypeptide. The C-terminal Cas9 fragment of a fusion protein can comprise a length of at least about: 100, 200, 300, 400, 500, 600, 700, 800, 900, 1000, 1100, 1200, or 1300 amino acids. The C-terminal Cas9 fragment of a fusion protein can comprise a sequence corresponding to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide. The N-terminal Cas9 fragment can comprise a sequence comprising at least: 85%, at least 90%, at least 91%, at least 92%, at least 93%, at least 94%, at least 95%, at least 96%, at least 97%, at least 98%, at least 99%, or at least 99.5% sequence identity to amino acid residues: 1099-1368, 918-1368, 906-1368, 780-1368, 765-1368, 718-1368, 94-1368, or 56-1368 as numbered in the above Cas9 reference sequence, or a corresponding amino acid residue in another Cas9 polypeptide.

The N-terminal Cas9 fragment and C-terminal Cas9 fragment of a fusion protein taken together may not correspond to a full-length naturally occurring Cas9 polypeptide sequence, for example, as set forth in the above Cas9 reference sequence.

The fusion protein described herein can effect targeted deamination with reduced deamination at non-target sites (e.g., off-target sites), such as reduced genome wide spurious deamination. The fusion protein described herein can effect targeted deamination with reduced bystander deamination at non-target sites. The undesired deamination or off-target deamination can be reduced by at least 30%, at least 40%, at least 50%, at least 60%, at least 70%, at least 80%, at least 90%, at least 95%, or at least 99% compared with, for example, an end terminus fusion protein comprising the deaminase fused to a N terminus or a C terminus of a Cas9 polypeptide. The undesired deamination or off-target deamination can be reduced by at least one-fold, at least two-fold, at least three-fold, at least four-fold, at least five-fold, at least tenfold, at least fifteen fold, at least twenty fold, at least thirty fold, at least forty fold, at least fifty fold, at least 60 fold, at least 70 fold, at least 80 fold, at least 90 fold, or at least hundred fold, compared with, for example, an end terminus fusion protein comprising the deaminase fused to a N terminus or a C terminus of a Cas9 polypeptide.

In some embodiments, the deaminase (e.g., adenosine deaminase, cytidine deaminase, or adenosine deaminase and cytidine deaminase) of the fusion protein deaminates no more than two nucleobases within the range of an R-loop. In some embodiments, the deaminase of the fusion protein deaminates no more than three nucleobases within the range of the R-loop. In some embodiments, the deaminase of the fusion protein deaminates no more than 2, 3, 4, 5, 6, 7, 8, 9, or 10 nucleobases within the range of the R-loop. An R-loop is a three-stranded nucleic acid structure including a DNA:RNA hybrid, a DNA:DNA or an RNA:RNA complementary structure and the associated with single-stranded DNA. As used herein, an R-loop may be formed when a target polynucleotide is contacted with a CRISPR complex or a base editing complex, wherein a portion of a guide polynucleotide, e.g. a guide RNA, hybridizes with and displaces with a portion of a target polynucleotide, e.g. a target DNA. In some embodiments, an R-loop comprises a hybridized region of a spacer sequence and a target DNA complementary sequence. An R-loop region may be of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleobase pairs in length. In some embodiments, the R-loop region is about 20 nucleobase pairs in length. It should be understood that, as used herein, an R-loop region is not limited to the target DNA strand that hybridizes with the guide polynucleotide. For example, editing of a target nucleobase within an R-loop region may be to a DNA strand that comprises the complementary strand to a guide RNA, or may be to a DNA strand that is the opposing strand of the strand complementary to the guide RNA. In some embodiments, editing in the region of the R-loop comprises editing a nucleobase on non-complementary strand (protospacer strand) to a guide RNA in a target DNA sequence.

The fusion protein described herein can effect target deamination in an editing window different from canonical base editing. In some embodiments, a target nucleobase is from about 1 to about 20 bases upstream of a PAM sequence in the target polynucleotide sequence. In some embodiments, a target nucleobase is from about 2 to about 12 bases upstream of a PAM sequence in the target polynucleotide sequence. In some embodiments, a target nucleobase is from about 1 to 9 base pairs, about 2 to 10 base pairs, about 3 to 11 base pairs, about 4 to 12 base pairs, about 5 to 13 base pairs, about 6 to 14 base pairs, about 7 to 15 base pairs, about 8 to 16 base pairs, about 9 to 17 base pairs, about 10 to 18 base pairs, about 11 to 19 base pairs, about 12 to 20 base pairs, about 1 to 7 base pairs, about 2 to 8 base pairs, about 3 to 9 base pairs, about 4 to 10 base pairs, about 5 to 11 base pairs, about 6 to 12 base pairs, about 7 to 13 base pairs, about 8 to 14 base pairs, about 9 to 15 base pairs, about 10 to 16 base pairs, about 11 to 17 base pairs, about 12 to 18 base pairs, about 13 to 19 base pairs, about 14 to 20 base pairs, about 1 to 5 base pairs, about 2 to 6 base pairs, about 3 to 7 base pairs, about 4 to 8 base pairs, about 5 to 9 base pairs, about 6 to 10 base pairs, about 7 to 11 base pairs, about 8 to 12 base pairs, about 9 to 13 base pairs, about 10 to 14 base pairs, about 11 to 15 base pairs, about 12 to 16 base pairs, about 13 to 17 base pairs, about 14 to 18 base pairs, about 15 to 19 base pairs, about 16 to 20 base pairs, about 1 to 3 base pairs, about 2 to 4 base pairs, about 3 to 5 base pairs, about 4 to 6 base pairs, about 5 to 7 base pairs, about 6 to 8 base pairs, about 7 to 9 base pairs, about 8 to 10 base pairs, about 9 to 11 base pairs, about 10 to 12 base pairs, about 11 to 13 base pairs, about 12 to 14 base pairs, about 13 to 15 base pairs, about 14 to 16 base pairs, about 15 to 17 base pairs, about 16 to 18 base pairs, about 17 to 19 base pairs, about 18 to 20 base pairs away or upstream of the PAM sequence. In some embodiments, a target nucleobase is about 1, 2, 3, 4, 5, 6, 7, 8, 9, 10, 11, 12, 13, 14, 15, 16, 17, 18, 19, 20, or more base pairs away from or upstream of the PAM sequence. In some embodiments, a target nucleobase is about 1, 2, 3, 4, 5, 6, 7, 8, or 9 base pairs upstream of the PAM sequence. In some embodiments, a target nucleobase is about 2, 3, 4, or 6 base pairs upstream of the PAM sequence.

The fusion protein can comprise more than one heterologous polypeptide. For example, the fusion protein can additionally comprise one or more UGI domains and/or one or more nuclear localization signals. The two or more heterologous domains can be inserted in tandem. The two or more heterologous domains can be inserted at locations such that they are not in tandem in the NapDNAbp.

A fusion protein can comprise a linker between the deaminase and the napDNAbp polypeptide. The linker can be a peptide or a non-peptide linker. For example, the linker can be an XTEN, (GGGS)n, (GGGGS)n, (G)n, (EAAAK)n, (GGS)n, SGSETPGTSESATPES. In some embodiments, the fusion protein comprises a linker between the N-terminal Cas9 fragment and the deaminase. In some embodiments, the fusion protein comprises a linker between the C-terminal Cas9 fragment and the deaminase. In some embodiments, the N-terminal and C-terminal fragments of napDNAbp are connected to the deaminase with a linker. In some embodiments, the N-terminal and C-terminal fragments are joined to the deaminase domain without a linker. In some embodiments, the fusion protein comprises a linker between the N-terminal Cas9 fragment and the deaminase, but does not comprise a linker between the C-terminal Cas9 fragment and the deaminase. In some embodiments, the fusion protein comprises a linker between the C-terminal Cas9 fragment and the deaminase, but does not comprise a linker between the N-terminal Cas9 fragment and the deaminase.

In some embodiments, the napDNAbp in the fusion protein is a Cas12 polypeptide, e.g., Cas12b/C2c1, or a fragment thereof. The Cas12 polypeptide can be a variant Cas12 polypeptide. In other embodiments, the N- or C-terminal fragments of the Cas12 polypeptide comprise a nucleic acid programmable DNA binding domain or a RuvC domain. In other embodiments, the fusion protein contains a linker between the Cas12 polypeptide and the catalytic domain. In other embodiments, the amino acid sequence of the linker is GGSGGS or GSSGSETPGTSESATPESSG. In other embodiments, the linker is a rigid linker. In other embodiments of the above aspects, the linker is encoded by GGAGGCTCTGGAGGAAGC or GGCTCTTCTGGATCTGAAACACCTGGCACAAGCGAGAGCGCCACCCCTGAGAGCTCTGGC.

Fusion proteins comprising a heterologous catalytic domain flanked by N- and C-terminal fragments of a Cas12 polypeptide are also useful for base editing in the methods as described herein. Fusion proteins comprising Cas12 and one or more deaminase domains, e.g., adenosine deaminase, or comprising an adenosine deaminase domain flanked by Cas12 sequences are also useful for highly specific and efficient base editing of target sequences. In an embodiment, a chimeric Cas12 fusion protein contains a heterologous catalytic domain (e.g., adenosine deaminase, cytidine deaminase, or adenosine deaminase and cytidine deaminase) inserted within a Cas12 polypeptide. In some embodiments, the fusion protein comprises an adenosine deaminase domain and a cytidine deaminase domain inserted within a Cas12. In some embodiments, an adenosine deaminase is fused within Cas12 and a cytidine deaminase is fused to the C-terminus. In some embodiments, an adenosine deaminase is fused within Cas12 and a cytidine deaminase fused to the N-terminus. In some embodiments, a cytidine deaminase is fused within Cas12 and an adenosine deaminase is fused to the C-terminus. In some embodiments, a cytidine deaminase is fused within Cas12 and an adenosine deaminase fused to the N-terminus. Exemplary structures of a fusion protein with an adenosine deaminase and a cytidine deaminase and a Cas12 are provided as follows:

NH₂-[Cas12(adenosine deaminase)]-[cytidine deaminase]-COOH;

NH₂-[cytidine deaminase]-[Cas12(adenosine deaminase)]-COOH;

NH₂-[Cas12(cytidine deaminase)]-[adenosine deaminase]-COOH; or

NH₂-[adenosine deaminase]-[Cas12(cytidine deaminase)]-COOH;

In some embodiments, the “-” used in the general architecture above indicates the presence of an optional linker.

In various embodiments, the catalytic domain has DNA modifying activity (e.g., deaminase activity), such as adenosine deaminase activity. In some embodiments, the adenosine deaminase is a TadA (e.g., TadA7.10). In some embodiments, the TadA is a TadA*8. In some embodiments, a TadA*8 is fused within Cas12 and a cytidine deaminase is fused to the C-terminus. In some embodiments, a TadA*8 is fused within Cas12 and a cytidine deaminase fused to the N-terminus. In some embodiments, a cytidine deaminase is fused within Cas12 and a TadA*8 is fused to the C-terminus. In some embodiments, a cytidine deaminase is fused within Cas12 and a TadA*8 fused to the N-terminus. Exemplary structures of a fusion protein with a TadA*8 and a cytidine deaminase and a Cas12 are provided as follows:

N-[Cas12(TadA*8)]-[cytidine deaminase]-C;

N-[cytidine deaminase]-[Cas12(TadA*8)]-C;

N-[Cas12(cytidine deaminase)]-[TadA*8]-C; or

N-[TadA*8]-[Cas12(cytidine deaminase)]-C.

In some embodiments, the “-” used in the general architecture above indicates the presence of an optional linker.

In other embodiments, the fusion protein contains one or more catalytic domains. In other embodiments, at least one of the one or more catalytic domains is inserted within the Cas12 polypeptide or is fused at the Cas12 N-terminus or C-terminus. In other embodiments, at least one of the one or more catalytic domains is inserted within a loop, an alpha helix region, an unstructured portion, or a solvent accessible portion of the Cas12 polypeptide. In other embodiments, the Cas12 polypeptide is Cas12a, Cas12b, Cas12c, Cas12d, Cas12e, Cas12g, Cas12h, or Cas12i. In other embodiments, the Cas12 polypeptide has at least about 85% amino acid sequence identity to Bacillus hisashii Cas12b, Bacillus thermoamylovorans Cas12b, Bacillus sp. V3-13 Cas12b, or Alicyclobacillus acidiphilus Cas12b. In other embodiments, the Cas12 polypeptide has at least about 90% amino acid sequence identity to Bacillus hisashii Cas12b, Bacillus thermoamylovorans Cas12b, Bacillus sp. V3-13 Cas12b, or Alicyclobacillus acidiphilus Cas12b. In other embodiments, the Cas12 polypeptide has at least about 95% amino acid sequence identity to Bacillus hisashii Cas12b, Bacillus thermoamylovorans Cas12b, Bacillus sp. V3-13 Cas12b, or Alicyclobacillus acidiphilus Cas12b. In other embodiments, the Cas12 polypeptide contains or consists essentially of a fragment of Bacillus hisashii Cas12b, Bacillus thermoamylovorans Cas12b, Bacillus sp. V3-13 Cas12b, or Alicyclobacillus acidiphilus Cas12b.

In other embodiments, the catalytic domain is inserted between amino acid positions 153-154, 255-256, 306-307, 980-981, 1019-1020, 534-535, 604-605, or 344-345 of BhCas12b or a corresponding amino acid residue of Cas12a, Cas12c, Cas12d, Cas12e, Cas12g, Cas12h, or Cas12i. In other embodiments, the catalytic domain is inserted between amino acids P153 and S154 of BhCas12b. In other embodiments, the catalytic domain is inserted between amino acids K255 and E256 of BhCas12b. In other embodiments, the catalytic domain is inserted between amino acids D980 and G981 of BhCas12b. In other embodiments, the catalytic domain is inserted between amino acids K1019 and L1020 of BhCas12b. In other embodiments, the catalytic domain is inserted between amino acids F534 and P535 of BhCas12b. In other embodiments, the catalytic domain is inserted between amino acids K604 and G605 of BhCas12b. In other embodiments, the catalytic domain is inserted between amino acids H344 and F345 of BhCas12b. In other embodiments, catalytic domain is inserted between amino acid positions 147 and 148, 248 and 249, 299 and 300, 991 and 992, or 1031 and 1032 of BvCas12b or a corresponding amino acid residue of Cas12a, Cas12c, Cas12d, Cas12e, Cas12g, Cas12h, or Cas12i. In other embodiments, the catalytic domain is inserted between amino acids P147 and D148 of BvCas12b. In other embodiments, the catalytic domain is inserted between amino acids G248 and G249 of BvCas12b. In other embodiments, the catalytic domain is inserted between amino acids P299 and E300 of BvCas12b. In other embodiments, the catalytic domain is inserted between amino acids G991 and E992 of BvCas12b. In other embodiments, the catalytic domain is inserted between amino acids K1031 and M1032 of BvCas12b. In other embodiments, the catalytic domain is inserted between amino acid positions 157 and 158, 258 and 259, 310 and 311, 1008 and 1009, or 1044 and 1045 of AaCas12b or a corresponding amino acid residue of Cas12a, Cas12c, Cas12d, Cas12e, Cas12g, Cas12h, or Cas12i. In other embodiments, the catalytic domain is inserted between amino acids P157 and G158 of AaCas12b. In other embodiments, the catalytic domain is inserted between amino acids V258 and G259 of AaCas12b. In other embodiments, the catalytic domain is inserted between amino acids D310 and P311 of AaCas12b. In other embodiments, the catalytic domain is inserted between amino acids G1008 and E1009 of AaCas12b. In other embodiments, the catalytic domain is inserted between amino acids G1044 and K1045 at of AaCas12b.

In other embodiments, the fusion protein contains a nuclear localization signal (e.g., a bipartite nuclear localization signal). In other embodiments, the amino acid sequence of the nuclear localization signal is MAPKKKRKVGIHGVPAA. In other embodiments of the above aspects, the nuclear localization signal is encoded by the following sequence: ATGGCCCCAAAGAAGAAGCGGAAGGTCGGTATCCACGGAGTCCCAGCAGCC. In other embodiments, the Cas12b polypeptide contains a mutation that silences the catalytic activity of a RuvC domain. In other embodiments, the Cas12b polypeptide contains D574A, D829A and/or D952A mutations. In other embodiments, the fusion protein further contains a tag (e.g., an influenza hemagglutinin tag).

In some embodiments, the fusion protein comprises a napDNAbp domain (e.g., Cas12-derived domain) with an internally fused nucleobase editing domain (e.g., all or a portion of a deaminase domain, e.g., an adenosine deaminase domain). In some embodiments, the napDNAbp is a Cas12b. In some embodiments, the base editor comprises a BhCas12b domain with an internally fused TadA*8 domain inserted at the loci provided in Table 6 below.

TABLE 6
Insertion loci in Cas12b proteins

| BhCas12b | Insertion site | Inserted between aa |
|---|---|---|
| position 1 | 153 | PS |
| position 2 | 255 | KE |
| position 3 | 306 | DE |
| position 4 | 980 | DG |
| position 5 | 1019 | KL |
| position 6 | 534 | FP |
| position 7 | 604 | KG |
| position 8 | 344 | HF |
| BvCas12b | Insertion site | Inserted between aa |
| position 1 | 147 | PD |
| position 2 | 248 | GG |
| position 3 | 299 | PE |
| position 4 | 991 | GE |
| position 5 | 1031 | KM |
| AaCas12b | Insertion site | Inserted between aa |
| position 1 | 157 | PG |
| position 2 | 258 | VG |
| position 3 | 310 | DP |
| position 4 | 1008 | GE |
| position 5 | 1044 | GK |

By way of nonlimiting example, an adenosine deaminase (e.g., ABE8.13) may be inserted into a BhCas12b to produce a fusion protein (e.g., ABE8.13-BhCas12b) that effectively edits a nucleic acid sequence.

In some embodiments, the base editing system described herein comprises an ABE with TadA inserted into a Cas9. Sequences of relevant ABEs with TadA inserted into a Cas9 are provided.

101 Cas9 TadAins 1015
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVGSSGSETPGTSESATPESSGSEVEFSHEYWMRHAL
TLAKRARDEREVPVGAVLVLNNRVIGEGWNRAIGLHDPTAHAEIMALRQG
GLVMQNYRLIDATLYVTFEPCVMCAGAMIHSRIGRVVFGVRNAKTGAAGS
LMDVLHYPGMNHRVEITEGILADECAALLCYFFRMPRQVFNAQKKAQSST
DYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
102 Cas9 TadAins 1022
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIGSSGSETPGTSESATPESSGSEVEFSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
103 Cas9 TadAins 1029
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGSSGSETPGTSESATPESSGS
EVEFSHEYWMRHALTLAKRARDEREVPVGAVLVLNNRVIGEGWNRAIGLH
DPTAHAEIMALRQGGLVMQNYRLIDATLYVTFEPCVMCAGAMIHSRIGRV
VFGVRNAKTGAAGSLMDVLHYPGMNHRVEITEGILADECAALLCYFFRMP
RQVFNAQKKAQSSTDGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
103 Cas9 TadAins 1040
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSGSSGSETPGT
SESATPESSGSEVEFSHEYWMRHALTLAKRARDEREVPVGAVLVLNNRVI
GEGWNRAIGLHDPTAHAEIMALRQGGLVMQNYRLIDATLYVTFEPCVMCA
GAMIHSRIGRVVFGVRNAKTGAAGSLMDVLHYPGMNHRVEITEGILADEC
AALLCYFFRMPRQVFNAQKKAQSSTDNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
105 Cas9 TadAins 1068
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGEGSSGSETPGTSESATPESSGSEVEFSHEYWMR
HALTLAKRARDEREVPVGAVLVLNNRVIGEGWNRAIGLHDPTAHAEIMAL
RQGGLVMQNYRLIDATLYVTFEPCVMCAGAMIHSRIGRVVFGVRNAKTGA
AGSLMDVLHYPGMNHRVEITEGILADECAALLCYFFRMPRQVFNAQKKAQ
SSTDTGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
106 Cas9 TadAins 1247
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGGSS
GSETPGTSESATPESSGSEVEFSHEYWMRHALTLAKRARDEREVPVGAVL
VLNNRVIGEGWNRAIGLHDPTAHAEIMALRQGGLVMQNYRLIDATLYVTF
EPCVMCAGAMIHSRIGRVVFGVRNAKTGAAGSLMDVLHYPGMNHRVEITE
GILADECAALLCYFFRMPRQVFNAQKKAQSSTDSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
107 Cas9 TadAins 1054
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGSSGSETPGTSESATPESSGSEVEFSHEYWMRHALTLAKRARDERE
VPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMN
HRVEITEGILADECAALLCYFFRMPRQVFNAQKKAQSSTDGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
108 Cas9 TadAins 1026
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEGSSGSETPGTSESATPESSGSEVE
FSHEYWMRHALTLAKRARDEREVPVGAVLVLNNRVIGEGWNRAIGLHDPT
AHAEIMALRQGGLVMQNYRLIDATLYVTFEPCVMCAGAMIHSRIGRVVFG
VRNAKTGAAGSLMDVLHYPGMNHRVEITEGILADECAALLCYFFRMPRQV
FNAQKKAQSSTDQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
109 Cas9 TadAins 768
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQGSSGSETPGTSESATPESSGSEVEFSHEYWMR
HALTLAKRARDEREVPVGAVLVLNNRVIGEGWNRAIGLHDPTAHAEIMAL
RQGGLVMQNYRLIDATLYVTFEPCVMCAGAMIHSRIGRVVFGVRNAKTGA
AGSLMDVLHYPGMNHRVEITEGILADECAALLCYFFRMPRTTQKGQKNSR
ERMKRIEEGIKELGSQILKEHPVENTQLQNEKLYLYYLQNGRDMYVDQEL
DINRLSDYDVDHIVPQSFLKDDSIDNKVLTRSDKNRGKSDNVPSEEVVKK
MKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQE
IGKATAKYFFYSNIMNFFKTEITLANGEIRKRPLIETNGETGEIVWDKGR
DFATVRKVLSMPQVNIVKKTEVQTGGFSKESILPKRNSDKLIARKKDWDP
KKYGGFDSPTVAYSVLVVAKVEKGKSKKLKSVKELLGITIMERSSFEKNP
IDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFD
TTIDRKRYTSTKEVLDATLIHQSITGLYETRIDLSQLGGD
110.1 Cas9 TadAins 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSETPGTSESATPESSGSEVEFSHEYWMRHALTLAKRARDEREVPVGA
VLVLNNRVIGEGWNRAIGLHDPTAHAEIMALRQGGLVMQNYRLIDATLYV
TFEPCVMCAGAMIHSRIGRVVFGVRNAKTGAAGSLMDVLHYPGMNHRVEI
TEGILADECAALLCYFFRMPREDNEQKQLFVEQHKHYLDEIIEQISEFSK
RVILADANLDKVLSAYNKHRDKPIREQAENIIHLFTLTNLGAPAAFKYFD
TTIDRKRYTSTKEVLDATLIHQSITGLYETRIDLSQLGGD
110.2 Cas9 TadAins 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSSGSETPGTSESATPESSGSEVEFSHEYWMRHALTLAKRARDEREVP
VGAVLVLNNRVIGEGWNRAIGLHDPTAHAEIMALRQGGLVMQNYRLIDAT
LYVTFEPCVMCAGAMIHSRIGRVVFGVRNAKTGAAGSLMDVLHYPGMNHR
VEITEGILADECAALLCYFFRMPREDNEQKQLFVEQHKHYLDEIIEQISE
FSKRVILADANLDKVLSAYNKHRDKPIREQAENIIHLFTLTNLGAPAAFK
YFDTTIDRKRYTSTKEVLDATLIHQSITGLYETRIDLSQLGGD
110.3 Cas9 TadAins 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSSGSETPGTSESATPESGSSSGSEVEFSHEYWMRHALTLAKRARDER
EVPVGAVLVLNNRVIGEGWNRAIGLHDPTAHAEIMALRQGGLVMQNYRLI
DATLYVTFEPCVMCAGAMIHSRIGRVVFGVRNAKTGAAGSLMDVLHYPGM
NHRVEITEGILADECAALLCYFFRMPREDNEQKQLFVEQHKHYLDEIIEQ
ISEFSKRVILADANLDKVLSAYNKHRDKPIREQAENIIHLFTLTNLGAPA
AFKYFDTTIDRKRYTSTKEVLDATLIHQSITGLYETRIDLSQLGGD
110.4 Cas9 TadAins 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIV+32TLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSSGSETPGTSESATPESGSSSGSEVEFSHEYWMRHALTLAKRARDER
EVPVGAVLVLNNRVIGEGWNRAIGLHDPTAHAEIMALRQGGLVMQNYRLI
DATLYVTFEPCVMCAGAMIHSRIGRVVFGVRNAKTGAAGSLMDVLHYPGM
NHRVEITEGILADECAALLCYFFRMRREDNEQKQLFVEQHKHYLDEIIEQ
ISEFSKRVILADANLDKVLSAYNKHRDKPIREQAENIIHLFTLTNLGAPA
AFKYFDTTIDRKRYTSTKEVLDATLIHQSITGLYETRIDLSQLGGD
110.5 Cas9 TadAins 1249
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSGS
SGSSGSETPGTSESATPESGSSSGSEVEFSHEYWMRHALTLAKRARDERE
VPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMN
HRVEITEGILADECAALLCYFFRMRRPEDNEQKQLFVEQHKHYLDEIIEQ
ISEFSKRVILADANLDKVLSAYNKHRDKPIREQAENIIHLFTLTNLGAPA
AFKYFDTTIDRKRYTSTKEVLDATLIHQSITGLYETRIDLSQLGGD
110.5 Cas9 TadAins delta 59-66 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIV+32TLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSSGSETPGTSESATPESGSSGSEVEFSHEYWMRHALTLAKRARDERE
VPVGAVLVLNNRVIGEGWNRAHAEIMALRQGGLVMQNYRLIDATLYVTFE
PCVMCAGAMIHSRIGRVVFGVRNAKTGAAGSLMDVLHYPGMNHRVEITEG
ILADECAALLCYFFRMPRQVFNAQKKAQSSTDEDNEQKQLFVEQHKHYLD
EIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGG
D
110.6 Cas9 TadAins 1251
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
GSSGSSGSETPGTSESATPESGSSSGSEVEFSHEYWMRHALTLAKRARDE
REVPVGAVLVLNNRVIGEGWNRAIGLHDPTAHAEIMALRQGGLVMQNYRL
IDATLYVTFEPCVMCAGAMIHSRIGRVVFGVRNAKTGAAGSLMDVLHYPG
MNHRVEITEGILADECAALLCYFFRMRRDNEQKQLFVEQHKHYLDEIIEQ
ISEFSKRVILADANLDKVLSAYNKHRDKPIREQAENIIHLFTLTNLGAPA
AFKYFDTTIDRKRYTSTKEVLDATLIHQSITGLYETRIDLSQLGGD
110.7 Cas9 TadAins 1252
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DGSSGSSGSETPGTSESATPESGSSSGSEVEFSHEYWMRHALTLAKRARD
EREVPVGAVLVLNNRVIGEGWNRAIGLHDPTAHAEIMALRQGGLVMQNYR
LIDATLYVTFEPCVMCAGAMIHSRIGRVVFGVRNAKTGAAGSLMDVLHYP
GMNHRVEITEGILADECAALLCYFFRMRRNEQKQLFVEQHKHYLDEIIEQ
ISEFSKRVILADANLDKVLSAYNKHRDKPIREQAENIIHLFTLTNLGAPA
AFKYFDTTIDRKRYTSTKEVLDATLIHQSITGLYETRIDLSQLGGD
110.8 Cas9 TadAins delta 59-66 C-truncate 1250
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPG
SSGSETPGTSESATPESSGSEVEFSHEYWMRHALTLAKRARDEREVPVGA
VLVLNNRVIGEGWNRAHAEIMALRQGGLVMQNYRLIDATLYVTFEPCVMC
AGAMIHSRIGRVVFGVRNAKTGAAGSLMDVLHYPGMNHRVEITEGILADE
CAALLCYFFRMPRQEDNEQKQLFVEQHKHYLDEIIEQISEFSKRVILADA
NLDKVLSAYNKHRDKPIREQAENIIHLFTLTNLGAPAAFKYFDTTIDRKR
YTSTKEVLDATLIHQSITGLYETRIDLSQLGGD
111.1 Cas9 TadAins 997
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDGSSGSETPGTSESATPESSGIKKYPKLESEFVYGDYKVYDVR
KMIAKSEQEIGKATAKYFFYSNIMNFFKTEITLANGEIRKRPLIETNGET
GEIVWDKGRDFATVRKVLSMPQVNIVKKTEVQTGGFSKESILPKRNSDKL
IARKKDWDPKKYGGFDSPTVAYSVLVVAKVEKGKSKKLKSVKELLGITIM
ERSSFEKNPIDFLEAKGYKEVKKDLIIKLPKYSLFELENGRKRMLASAGE
LQKGNELALPSKYVNFLYLASHYEKLKGSPEDNEQKQLFVEQHKHYLDEI
IEQISEFSKRVILADANLDKVLSAYNKHRDKPIREQAENIIHLFTLTNLG
APAAFKYFDTTIDRKRYTSTKEVLDATLIHQSITGLYETRIDLSQLGGD
111.2 Cas9 TadAins 997
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDGSSGSSGSETPGTSESATPESSGGSSIKKYPKLESEFVYGDY
KVYDVRKMIAKSEQEIGKATAKYFFYSNIMNFFKTEITLANGEIRKRPLI
ETNGETGEIVWDKGRDFATVRKVLSMPQVNIVKKTEVQTGGFSKESILPK
RNSDKLIARKKDWDPKKYGGFDSPTVAYSVLVVAKVEKGKSKKLKSVKEL
LGITIMERSSFEKNPIDFLEAKGYKEVKKDLIIKLPKYSLFELENGRKRM
LASAGELQKGNELALPSKYVNFLYLASHYEKLKGSPEDNEQKQLFVEQHK
HYLDEIIEQISEFSKRVILADANLDKVLSAYNKHRDKPIREQAENIIHLF
TLTNLGAPAAFKYFDTTIDRKRYTSTKEVLDATLIHQSITGLYETRIDLS
QLGGD
112 delta HNH TadA
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSEVEFSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDGGLSELDKAGFIKRQLVETRQITKHVAQILDSRMNTKYDEND
KLIREVKVITLKSKLVSDFRKDFQFYKVREINNYHHAHDAYLNAVVGTAL
IKKYPKLESEFVYGDYKVYDVRKMIAKSEQEIGKATAKYFFYSNIMNFFK
TEITLANGEIRKRPLIETNGETGEIVWDKGRDFATVRKVLSMPQVNIVKK
TEVQTGGFSKESILPKRNSDKLIARKKDWDPKKYGGFDSPTVAYSVLVVA
KVEKGKSKKLKSVKELLGITIMERSSFEKNPIDFLEAKGYKEVKKDLIIK
LPKYSLFELENGRKRMLASAGELQKGNELALPSKYVNFLYLASHYEKLKG
SPEDNEQKQLFVEQHKHYLDEIIEQISEFSKRVILADANLDKVLSAYNKH
RDKPIREQAENIIHLFTLTNLGAPAAFKYFDTTIDRKRYTSTKEVLDATL
IHQSITGLYETRIDLSQLGGD
113 N-term single TadA helix trunc 165-end
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRSGGSSGGSSGSETPGTSESATPESSGGSSGGSDKKYSIGLAIGTNSV
GWAVITDEYKVPSKKFKVLGNTDRHSIKKNLIGALLFDSGETAEATRLKR
TARRRYTRRKNRICYLQEIFSNEMAKVDDSFFHRLEESFLVEEDKKHERH
PIFGNIVDEVAYHEKYPTIYHLRKKLVDSTDKADLRLIYLALAHMIKFRG
HFLIEGDLNPDNSDVDKLFIQLVQTYNQLFEENPINASGVDAKAILSARL
SKSRRLENLIAQLPGEKKNGLFGNLIALSLGLTPNFKSNFDLAEDAKLQL
SKDTYDDDLDNLLAQIGDQYADLFLAAKNLSDAILLSDILRVNTEITKAP
LSASMIKRYDEHHQDLTLLKALVRQQLPEKYKEIFFDQSKNGYAGYIDGG
ASQEEFYKFIKPILEKMDGTEELLVKLNREDLLRKQRTFDNGSIPHQIHL
GELHAILRRQEDFYPFLKDNREKIEKILTFRIPYYVGPLARGNSRFAWMT
RKSEETITPWNFEEVVDKGASAQSFIERMTNFDKNLPNEKVLPKHSLLYE
YFTVYNELTKVKYVTEGMRKPAFLSGEQKKAIVDLLFKTNRKVTVKQLKE
DYFKKIECFDSVEISGVEDRFNASLGTYHDLLKIIKDKDFLDNEENEDIL
EDIVLTLTLFEDREMIEERLKTYAHLFDDKVMKQLKRRRYTGWGRLSRKL
INGIRDKQSGKTILDFLKSDGFANRNFMQLIHDDSLTFKEDIQKAQVSGQ
GDSLHEHIANLAGSPAIKKGILQTVKVVDELVKVMGRHKPENIVIEMARE
NQTTQKGQKNSRERMKRIEEGIKELGSQILKEHPVENTQLQNEKLYLYYL
QNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDF
RKDFQFYKVREINNYHHAHDAYLNAVVGTALIKKYPKLESEFVYGDYKVY
DVRKMIAKSEQEIGKATAKYFFYSNIMNFFKTEITLANGEIRKRPLIETN
GETGEIVWDKGRDFATVRKVLSMPQVNIVKKTEVQTGGFSKESILPKRNS
DKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYL
DEIIEQISEFSKRVILADANLDKVLSAYNKHRDKPIREQAENIIHLFTLT
NLGAPAAFKYFDTTIDRKRYTSTKEVLDATLIHQSITGLYETRIDLSQLG
GD
114 N-term single TadA helix trunc 165-end delta
59-65
MSEVEFSHEYWMRHALTLAKRARDEREVPVGAVLVLNNRVIGEGWNRTAH
AEIMALRQGGLVMQNYRLIDATLYVTFEPCVMCAGAMIHSRIGRVVFGVR
NAKTGAAGSLMDVLHYPGMNHRVEITEGILADECAALLCYFFRMPRSGGS
SGGSSGSETPGTSESATPESSGGSSGGSDKKYSIGLAIGTNSVGWAVITD
EYKVPSKKFKVLGNTDRHSIKKNLIGALLFDSGETAEATRLKRTARRRYT
RRKNRICYLQEIFSNEMAKVDDSFFHRLEESFLVEEDKKHERHPIFGNIV
DEVAYHEKYPTIYHLRKKLVDSTDKADLRLIYLALAHMIKFRGHFLIEGD
LNPDNSDVDKLFIQLVQTYNQLFEENPINASGVDAKAILSARLSKSRRLE
NLIAQLPGEKKNGLFGNLIALSLGLTPNFKSNFDLAEDAKLQLSKDTYDD
DLDNLLAQIGDQYADLFLAAKNLSDAILLSDILRVNTEITKAPLSASMIK
RYDEHHQDLTLLKALVRQQLPEKYKEIFFDQSKNGYAGYIDGGASQEEFY
KFIKPILEKMDGTEELLVKLNREDLLRKQRTFDNGSIPHQIHLGELHAIL
RRQEDFYPFLKDNREKIEKILTFRIPYYVGPLARGNSRFAWMTRKSEETI
TPWNFEEVVDKGASAQSFIERMTNFDKNLPNEKVLPKHSLLYEYFTVYNE
LTKVKYVTEGMRKPAFLSGEQKKAIVDLLFKTNRKVTVKQLKEDYFKKIE
CFDSVEISGVEDRFNASLGTYHDLLKIIKDKDFLDNEENEDILEDIVLTL
TLFEDREMIEERLKTYAHLFDDKVMKQLKRRRYTGWGRLSRKLINGIRDK
QSGKTILDFLKSDGFANRNFMQLIHDDSLTFKEDIQKAQVSGQGDSLHEH
IANLAGSPAIKKGILQTVKVVDELVKVMGRHKPENIVIEMARENQTTQKG
QKNSRERMKRIEEGIKELGSQILKEHPVENTQLQNEKLYLYYLQNGRDMY
VDQELDINRLSDYDVDHIVPQSFLKDDSIDNKVLTRSDKNRGKSDNVPSE
EVVKKMKNYWRQLLNAKLITQRKFDNLTKAERGGLSELDKAGFIKRQLVE
TRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIA
KSEQEIGKATAKYFFYSNIMNFFKTEITLANGEIRKRPLIETNGETGEIV
WDKGRDFATVRKVLSMPQVNIVKKTEVQTGGFSKESILPKRNSDKLIARK
KDWDPKKYGGFDSPTVAYSVLVVAKVEKGKSKKLKSVKELLGITIMERSS
FEKNPIDFLEAKGYKEVKKDLIIKLPKYSLFELENGRKRMLASAGELQKG
NELALPSKYVNFLYLASHYEKLKGSPEDNEQKQLFVEQHKHYLDEIIEQI
SEFSKRVILADANLDKVLSAYNKHRDKPIREQAENIIHLFTLTNLGAPAA
FKYFDTTIDRKRYTSTKEVLDATLIHQSITGLYETRIDLSQLGGD
115.1 Cas9 TadAins1004
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIV+32TLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKGSSGSETPGTSESATPESSGSEVEFSHEYWMRHALTLAKRARDEREV
PVGAVLVLNNRVIGEGWNRAIGLHDPTAHAEIMALRQGGLVMQNYRLIDA
TLYVTFEPCVMCAGAMIHSRIGRVVFGVRNAKTGAAGSLMDVLHYPGMNH
RVEITEGILADECAALLCYFFRMPRQLESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
115.2 Cas9 TadAins1005
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLGSSGSETPGTSESATPESSGSEVEFSHEYWMRHALTLAKRARDERE
VPVGAVLVLNNRVIGEGWNRAIGLHDPTAHAEIMALRQGGLVMQNYRLID
ATLYVTFEPCVMCAGAMIHSRIGRVVFGVRNAKTGAAGSLMDVLHYPGMN
HRVEITEGILADECAALLCYFFRMPRQESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
115.3 Cas9 TadAins1006
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLEGSSGSETPGTSESATPESSGSEVEFSHEYWMRHALTLAKRARDER
EVPVGAVLVLNNRVIGEGWNRAIGLHDPTAHAEIMALRQGGLVMQNYRLI
DATLYVTFEPCVMCAGAMIHSRIGRVVFGVRNAKTGAAGSLMDVLHYPGM
NHRVEITEGILADECAALLCYFFRMPRQSEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
115.4 Cas9 TadAins1007
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESGSSGSETPGTSESATPESSGSEVEFSHEYWMRHALTLAKRARDE
REVPVGAVLVLNNRVIGEGWNRAIGLHDPTAHAEIMALRQGGLVMQNYRL
IDATLYVTFEPCVMCAGAMIHSRIGRVVFGVRNAKTGAAGSLMDVLHYPG
MNHRVEITEGILADECAALLCYFFRMPRQEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
116.1 Cas9 TadAins C-term truncate2 792
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGGSSGSETP
GTSESATPESSGSEVEFSHEYWMRHALTLAKRARDEREVPVGAVLVLNNR
VIGEGWNRAIGLHDPTAHAEIMALRQGGLVMQNYRLIDATLYVTFEPCVM
CAGAMIHSRIGRVVFGVRNAKTGAAGSLMDVLHYPGMNHRVEITEGILAD
ECAALLCYFFRMPRQSQILKEHPVENTQLQNEKLYLYYLQNGRDMYVDQE
LDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
116.2 Cas9 TadAins C-term truncate2 791
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSSGSETPG
TSESATPESSGSEVEFSHEYWMRHALTLAKRARDEREVPVGAVLVLNNRV
IGEGWNRAIGLHDPTAHAEIMALRQGGLVMQNYRLIDATLYVTFEPCVMC
AGAMIHSRIGRVVFGVRNAKTGAAGSLMDVLHYPGMNHRVEITEGILADE
CAALLCYFFRMPRQGSQILKEHPVENTQLQNEKLYLYYLQNGRDMYVDQE
LDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
116.3 Cas9 TadAins C-term truncate2 790
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKEGSSGSETPGT
SESATPESSGSEVEFSHEYWMRHALTLAKRARDEREVPVGAVLVLNNRVI
GEGWNRAIGLHDPTAHAEIMALRQGGLVMQNYRLIDATLYVTFEPCVMCA
GAMIHSRIGRVVFGVRNAKTGAAGSLMDVLHYPGMNHRVEITEGILADEC
AALLCYFFRMPRQLGSQILKEHPVENTQLQNEKLYLYYLQNGRDMYVDQE
LDINRLSDYDVDHIVPQSFLKDDSIDNKVLTRSDKNRGKSDNVPSEEVVK
KMKNYWRQLLNAKLITQRKFDNLTKAERGGLSELDKAGFIKRQLVETRQI
TKHVAQILDSRMNTKYDENDKLIREVKVITLKSKLVSDFRKDFQFYKVRE
INNYHHAHDAYLNAVVGTALIKKYPKLESEFVYGDYKVYDVRKMIAKSEQ
EIGKATAKYFFYSNIMNFFKTEITLANGEIRKRPLIETNGETGEIVWDKG
RDFATVRKVLSMPQVNIVKKTEVQTGGFSKESILPKRNSDKLIARKKDWD
PKKYGGFDSPTVAYSVLVVAKVEKGKSKKLKSVKELLGITIMERSSFEKN
PIDFLEAKGYKEVKKDLIIKLPKYSLFELENGRKRMLASAGELQKGNELA
LPSKYVNFLYLASHYEKLKGSPEDNEQKQLFVEQHKHYLDEIIEQISEFS
KRVILADANLDKVLSAYNKHRDKPIREQAENIIHLFTLTNLGAPAAFKYF
DTTIDRKRYTSTKEVLDATLIHQSITGLYETRIDLSQLGGD
117 Cas9 delta 1017-1069
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYSSGSEVEFSHEYWMRHALTLAKRARDEREVPVGA
VLVLNNRVIGEGWNRAIGLHDPTAHAEIMALRQGGLVMQNYRLIDATLYV
TFEPCVMCAGAMIHSRIGRVVFGVRNAKTGAAGSLMDVLHYPGMNHRVEI
TEGILADECAALLCYFFRMPRQVFNAQKKAQSSTDGEIVWDKGRDFATVR
KVLSMPQVNIVKKTEVQTGGFSKESILPKRNSDKLIARKKDWDPKKYGGF
DSPTVAYSVLVVAKVEKGKSKKLKSVKELLGITIMERSSFEKNPIDFLEA
KGYKEVKKDLIIKLPKYSLFELENGRKRMLASAGELQKGNELALPSKYVN
FLYLASHYEKLKGSPEDNEQKQLFVEQHKHYLDEIIEQISEFSKRVILAD
ANLDKVLSAYNKHRDKPIREQAENIIHLFTLTNLGAPAAFKYFDTTIDRK
RYTSTKEVLDATLIHQSITGLYETRIDLSQLGGD
118 Cas9 TadA-CP116ins 1067
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNMNHRVEITEGILADECAALLCYFFRMPRQVFNAQ
KKAQSSTDGSSGSETPGTSESATPESSGSEVEFSHEYWMRHALTLAKRAR
DEREVPVGAVLVLNNRVIGEGWNRAIGLHDPTAHAEIMALRQGGLVMQNY
RLIDATLYVTFEPCVMCAGAMIHSRIGRVVFGVRNAKTGAAGSLMDVLHY
PGGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
119 Cas9 TadAins 701
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SGSSGSETPGTSESATPESSGSEVEFSHEYWMRHALTLAKRARDEREVPV
GAVLVLNNRVIGEGWNRAIGLHDPTAHAEIMALRQGGLVMQNYRLIDATL
YVTFEPCVMCAGAMIHSRIGRVVFGVRNAKTGAAGSLMDVLHYPGMNHRV
EITEGILADECAALLCYFFRMPRQVFNAQKKAQSSTDLTFKEDIQKAQVS
GQGDSLHEHIANLAGSPAIKKGILQTVKVVDELVKVMGRHKPENIVIEMA
RENQTTQKGQKNSRERMKRIEEGIKELGSQILKEHPVENTQLQNEKLYLY
YLQNGRDMYVDQELDINRLSDYDVDHIVPQSFLKDDSIDNKVLTRSDKNR
GKSDNVPSEEVVKKMKNYWRQLLNAKLITQRKFDNLTKAERGGLSELDKA
GFIKRQLVETRQITKHVAQILDSRMNTKYDENDKLIREVKVITLKSKLVS
DFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
120 Cas9 TadACP136ins 1248
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSMN
HRVEITEGILADECAALLCYFFRMPRQVFNAQKKAQSSTDGSSGSETPGT
SESATPESSGSEVEFSHEYWMRHALTLAKRARDEREVPVGAVLVLNNRVI
GEGWNRAIGLHDPTAHAEIMALRQGGLVMQNYRLIDATLYVTFEPCVMCA
GAMIHSRIGRVVFGVRNAKTGAAGSLMDVLHYPGPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
121 Cas9 TadACP136ins 1052
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLAMNHRVEITEGILADECAALLCYFFRMPRQVFNAQKKAQSSTDGSSGS
EIPGTSESATPESSGSEVEFSHEYWMRHALTLAKRARDEREVPVGAVLVL
NNRVIGEGWNRAIGLHDPTAHAEIMALRQGGLVMQNYRLIDATLYVTFEP
CVMCAGAMIHSRIGRVVFGVRNAKTGAAGSLMDVLHYPGNGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
122 Cas9 TadACP136ins 1041
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSMNHRVEITEG
ILADECAALLCYFFRMPRQVFNAQKKAQSSTDGSSGSETPGTSESATPES
SGSEVEFSHEYWMRHALTLAKRARDEREVPVGAVLVLNNRVIGEGWNRAI
GLHDPTAHAEIMALRQGGLVMQNYRLIDATLYVTFEPCVMCAGAMIHSRI
GRVVFGVRNAKTGAAGSLMDVLHYPGNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
123 Cas9 TadACP139ins 1299
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRMN
HRVEITEGILADECAALLCYFFRMPRQVFNAQKKAQSSTDGSSGSETPGT
SESATPESSGSEVEFSHEYWMRHALTLAKRARDEREVPVGAVLVLNNRVI
GEGWNRAIGLHDPTAHAEIMALRQGGLVMQNYRLIDATLYVTFEPCVMCA
GAMIHSRIGRVVFGVRNAKTGAAGSLMDVLHYPGDKPIREQAENIIHLFT
LTNLGAPAAFKYFDTTIDRKRYTSTKEVLDATLIHQSITGLYETRIDLSQ
LGGD
124 Cas9 delta 792-872 TadAins
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSEVEFSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDEEVVKKMKNYWRQLLNAKLITQRKFDNLTKAERGGLSELDKA
GFIKRQLVETRQITKHVAQILDSRMNTKYDENDKLIREVKVITLKSKLVS
DFRKDFQFYKVREINNYHHAHDAYLNAVVGTALIKKYPKLESEFVYGDYK
VYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
125 Cas9 delta 792-906 TadAins
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSEVEFSHE
YWMRHALTLAKRARDEREVPVGAVLVLNNRVIGEGWNRAIGLHDPTAHAE
IMALRQGGLVMQNYRLIDATLYVTFEPCVMCAGAMIHSRIGRVVFGVRNA
KTGAAGSLMDVLHYPGMNHRVEITEGILADECAALLCYFFRMPRQVFNAQ
KKAQSSTDGLSELDKAGFIKRQLVETRQITKHVAQILDSRMNTKYDENDK
LIREVKVITLKSKLVSDFRKDFQFYKVREINNYHHAHDAYLNAVVGTALI
KKYPKLESEFVYGDYKVYDVRKMIAKSEQEIGKATAKYFFYSNIMNFFKT
EITLANGEIRKRPLIETNGETGEIVWDKGRDFATVRKVLSMPQVNIVKKT
EVQTGGFSKESILPKRNSDKLIARKKDWDPKKYGGFDSPTVAYSVLVVAK
VEKGKSKKLKSVKELLGITIMERSSFEKNPIDFLEAKGYKEVKKDLIIKL
PKYSLFELENGRKRMLASAGELQKGNELALPSKYVNFLYLASHYEKLKGS
PEDNEQKQLFVEQHKHYLDEIIEQISEFSKRVILADANLDKVLSAYNKHR
DKPIREQAENIIHLFTLTNLGAPAAFKYFDTTIDRKRYTSTKEVLDATLI
HQSITGLYETRIDLSQLGGD
126 TadA CP65ins 1003
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKTAHAEIMALRQGGLVMQNYRLIDATLYVTFEPCVMCAGAMIHSRIGR
VVFGVRNAKTGAAGSLMDVLHYPGMNHRVEITEGILADECAALLCYFFRM
PRQVFNAQKKAQSSTDGSSGSETPGTSESATPESSGSEVEFSHEYWMRHA
LTLAKRARDEREVPVGAVLVLNNRVIGEGWNRAIGLHDPLESEFVYGDYK
VYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
127 TadA CP65ins 1016
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVTAHAEIMALRQGGLVMQNYRLIDATLYVTFEPCVM
CAGAMIHSRIGRVVFGVRNAKTGAAGSLMDVLHYPGMNHRVEITEGILAD
ECAALLCYFFRMPRQVFNAQKKAQSSTDGSSGSETPGTSESATPESSGSE
VEFSHEYWMRHALTLAKRARDEREVPVGAVLVLNNRVIGEGWNRAIGLHD
PYDVRKMIAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
128 TadA CP65ins 1022
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMITAHAEIMALRQGGLVMQNYRLIDATLYV
TFEPCVMCAGAMIHSRIGRVVFGVRNAKTGAAGSLMDVLHYPGMNHRVEI
TEGILADECAALLCYFFRMPRQVFNAQKKAQSSTDGSSGSETPGTSESAT
PESSGSEVEFSHEYWMRHALTLAKRARDEREVPVGAVLVLNNRVIGEGWN
RAIGLHDPAKSEQEIGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
129 TadA CP65ins 1029
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEITAHAEIMALRQGGLVMQNYRL
IDATLYVTFEPCVMCAGAMIHSRIGRVVFGVRNAKTGAAGSLMDVLHYPG
MNHRVEITEGILADECAALLCYFFRMPRQVFNAQKKAQSSTDGSSGSETP
GTSESATPESSGSEVEFSHEYWMRHALTLAKRARDEREVPVGAVLVLNNR
VIGEGWNRAIGLHDPGKATAKYFFYSNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
130 TadA CP65ins 1041
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSTAHAEIMALR
QGGLVMQNYRLIDATLYVTFEPCVMCAGAMIHSRIGRVVFGVRNAKTGAA
GSLMDVLHYPGMNHRVEITEGILADECAALLCYFFRMPRQVFNAQKKAQS
STDGSSGSETPGTSESATPESSGSEVEFSHEYWMRHALTLAKRARDEREV
PVGAVLVLNNRVIGEGWNRAIGLHDPNIMNFFKTEITLANGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
131 TadA CP65ins 1054
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTDGSSGSETPGTSESATPESSGSEVEFSHEYWMRH
ALTLAKRARDEREVPVGAVLVLNNRVIGEGWNRAIGLHDPGEIRKRPLIE
TNGETGEIVWDKGRDFATVRKVLSMPQVNIVKKTEVQTGGFSKESILPKR
NSDKLIARKKDWDPKKYGGFDSPTVAYSVLVVAKVEKGKSKKLKSVKELL
GITIMERSSFEKNPIDFLEAKGYKEVKKDLIIKLPKYSLFELENGRKRML
ASAGELQKGNELALPSKYVNFLYLASHYEKLKGSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD
132 TadA CP65ins 1246
MDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGTAH
AEIMALRQGGLVMQNYRLIDATLYVTFEPCVMCAGAMIHSRIGRVVFGVR
NAKTGAAGSLMDVLHYPGMNHRVEITEGILADECAALLCYFFRMPRQVFN
AQKKAQSSTDGSSGSETPGTSESATPESSGSEVEFSHEYWMRHALTLAKR
ARDEREVPVGAVLVLNNRVIGEGWNRAIGLHDPSPEDNEQKQLFVEQHKH
YLDEIIEQISEFSKRVILADANLDKVLSAYNKHRDKPIREQAENIIHLFT
LTNLGAPAAFKYFDTTIDRKRYTSTKEVLDATLIHQSITGLYETRIDLSQ
LGGD

In some embodiments, adenosine deaminase base editors were generated to insert TadA or variants thereof into the Cas9 polypeptide at the identified positions.

Exemplary, yet nonlimiting, fusion proteins are described in International PCT Application Nos. PCT/US2020/016285 and U.S. Provisional Application Nos. 62/852,228 and 62/852,224, the contents of which are incorporated by reference herein in their entireties.

### Nucleobase Editing Domain

Described herein are base editors comprising a fusion protein that includes a polynucleotide programmable nucleotide binding domain and a nucleobase editing domain (e.g., a deaminase domain). The base editor can be programmed to edit one or more bases in a target polynucleotide sequence by interacting with a guide polynucleotide capable of recognizing the target sequence. Once the target sequence has been recognized, the base editor is anchored on the polynucleotide where editing is to occur and the deaminase domain components of the base editor can then edit a target base.

In some embodiments, the nucleobase editing domain includes a deaminase domain. As particularly described herein, the deaminase domain includes a cytosine deaminase or an adenosine deaminase. In some embodiments, the terms “cytosine deaminase” and “cytidine deaminase” can be used interchangeably. In some embodiments, the terms “adenine deaminase” and “adenosine deaminase” can be used interchangeably. Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

### A to G Editing

In some embodiments, a base editor described herein can comprise a deaminase domain which includes an adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA).

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein can comprise a Cas9 domain that has reduced nuclease activity. In some embodiments, the fusion proteins provided herein can have a Cas9 domain that does not have nuclease activity (dCas9), or a Cas9 domain that cuts one strand of a duplexed DNA molecule, referred to as a Cas9 nickase (nCas9). Without wishing to be bound by any particular theory, the presence of the catalytic residue (e.g., H840) maintains the activity of the Cas9 to cleave the non-edited (e.g., non-deaminated) strand containing a T opposite the targeted A. Mutation of the catalytic residue (e.g., D10 to A10) of Cas9 prevents cleavage of the edited strand containing the targeted A residue. Such Cas9 variants are able to generate a single-strand DNA break (nick) at a specific location based on the gRNA-defined target sequence, leading to repair of the non-edited strand, ultimately resulting in a T to C change on the non-edited strand. In some embodiments, an A-to-G base editor further comprises an inhibitor of inosine base excision repair, for example, a uracil glycosylase inhibitor (UGI) domain or a catalytically inactive inosine specific nuclease. Without wishing to be bound by any particular theory, the UGI domain or catalytically inactive inosine specific nuclease can inhibit or prevent base excision repair of a deaminated adenosine residue (e.g., inosine), which can improve the activity or efficiency of the base editor.

A base editor comprising an adenosine deaminase can act on any polynucleotide, including DNA, RNA and DNA-RNA hybrids. In certain embodiments, a base editor comprising an adenosine deaminase can deaminate a target A of a polynucleotide comprising RNA. For example, the base editor can comprise an adenosine deaminase domain capable of deaminating a target A of an RNA polynucleotide and/or a DNA-RNA hybrid polynucleotide. In an embodiment, an adenosine deaminase incorporated into a base editor comprises all or a portion of adenosine deaminase acting on RNA (ADAR, e.g., ADAR1 or ADAR2). In another embodiment, an adenosine deaminase incorporated into a base editor comprises all or a portion of adenosine deaminase acting on tRNA (ADAT). A base editor comprising an adenosine deaminase domain can also be capable of deaminating an A nucleobase of a DNA polynucleotide. In an embodiment an adenosine deaminase domain of a base editor comprises all or a portion of an ADAT comprising one or more mutations which permit the ADAT to deaminate a target A in DNA. For example, the base editor can comprise all or a portion of an ADAT from Escherichia coli (EcTadA) comprising one or more of the following mutations: D108N, A106V, D147Y, E155V, L84F, H123Y, I156F, or a corresponding mutation in another adenosine deaminase.

The adenosine deaminase can be derived from any suitable organism (e.g., E. coli). In some embodiments, the adenine deaminase is a naturally-occurring adenosine deaminase that includes one or more mutations corresponding to any of the mutations provided herein (e.g., mutations in ecTadA). The corresponding residue in any homologous protein can be identified by e.g., sequence alignment and determination of homologous residues. The mutations in any naturally-occurring adenosine deaminase (e.g., having homology to ecTadA) that corresponds to any of the mutations described herein (e.g., any of the mutations identified in ecTadA) can be generated accordingly.

### Adenosine Deaminases

In some embodiments, fusion proteins described herein can comprise a deaminase domain which includes an adenosine deaminase. Such an adenosine deaminase domain of a base editor can facilitate the editing of an adenine (A) nucleobase to a guanine (G) nucleobase by deaminating the A to form inosine (I), which exhibits base pairing properties of G. Adenosine deaminase is capable of deaminating (i.e., removing an amine group) adenine of a deoxyadenosine residue in deoxyribonucleic acid (DNA).

In some embodiments, the adenosine deaminases provided herein are capable of deaminating adenine. In some embodiments, the adenosine deaminases provided herein are capable of deaminating adenine in a deoxyadenosine residue of DNA. In some embodiments, the adenine deaminase is a naturally-occurring adenosine deaminase that includes one or more mutations corresponding to any of the mutations provided herein (e.g., mutations in ecTadA). One of skill in the art will be able to identify the corresponding residue in any homologous protein, e.g., by sequence alignment and determination of homologous residues. Accordingly, one of skill in the art would be able to generate mutations in any naturally-occurring adenosine deaminase (e.g., having homology to ecTadA) that corresponds to any of the mutations described herein, e.g., any of the mutations identified in ecTadA. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

The disclosure provides adenosine deaminase variants that have increased efficiency (>50-60%) and specificity. In particular, the adenosine deaminase variants described herein are more likely to edit a desired base within a polynucleotide, and are less likely to edit bases that are not intended to be altered (i.e., “bystanders”).

In particular embodiments, the TadA is any one of the TadA described in PCT/US2017/045381 (WO 2018/027078), which is incorporated herein by reference in its entirety.

In some embodiments, the nucleobase editors of the disclosure are adenosine deaminase variants comprising an alteration in the following sequence:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD(also termed TadA*7.10).

In particular embodiments, the fusion proteins comprise a single (e.g., provided as a monomer) TadA*8 variant. In some embodiments, the TadA*8 is linked to a Cas9 nickase. In some embodiments, the fusion proteins of the disclosure comprise as a heterodimer of a wild-type TadA (TadA(wt)) linked to a TadA*8 variant. In other embodiments, the fusion proteins of the disclosure comprise as a heterodimer of a TadA*7.10 linked to a TadA*8 variant. In some embodiments, the base editor is ABE8 comprising a TadA*8 variant monomer. In some embodiments, the base editor is ABE8 comprising a heterodimer of a TadA*8 variant and a TadA(wt). In some embodiments, the base editor is ABE8 comprising a heterodimer of a TadA*8 variant and TadA*7.10. In some embodiments, the base editor is ABE8 comprising a heterodimer of a TadA*8 variant. In some embodiments, the TadA*8 variant is selected from Table 8. In some embodiments, the ABE8 is selected from Table 8, 9, or 10. The relevant sequences follow:

Wild-type TadA (TadA(wt)) or “the TadA reference
sequence”
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD
TadA*7.10:
MSEVEFSHEYW MRHALTLAKR ARDEREVPVG AVLVLNNRVI
GEGWNRAIGL HDPTAHAEIM ALRQGGLVMQ NYRLIDATLY
VTFEPCVMCA GAMIHSRIGR VVFGVRNAKT GAAGSLMDVL
HYPGMNHRVE ITEGILADEC AALLCYFFRM PRQVFNAQKK
AQSSTD

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In some embodiments the TadA deaminase is a full-length E. coli TadA deaminase. For example, in certain embodiments, the adenosine deaminase comprises the amino acid sequence:

MRRAFITGVFFLSEVEFSHEYWMRHALTLAKRAWDEREVPVGAVLVHNNR
VIGEGWNRPIGRHDPTAHAEIMALRQGGLVMQNYRLIDATLYVTLEPCVM
CAGAMIHSRIGRVVFGARDAKTGAAGSLMDVLHHPGMNHRVEITEGILAD
ECAALLSDFFRMRRQEIKAQKKAQSSTD.

It should be appreciated, however, that additional adenosine deaminases useful in the present application would be apparent to the skilled artisan and are within the scope of this disclosure. For example, the adenosine deaminase may be a homolog of adenosine deaminase acting on tRNA (ADAT). Without limitation, the amino acid sequences of exemplary AD AT homologs include the following:

Staphylococcus aureus TadA:
MGSHMTNDIYFMTLAIEEAKKAAQLGEVPIGAIITKDDEVIARAHNLRET
LQQPTAHAEHIAIERAAKVLGSWRLEGCTLYVTLEPCVMCAGTIVMSRIP
RVVYGADDPKGGCSGSLMNLLQQSNFNHRAIVDKGVLKEACSTLLTTFFK
NLRANKKSTN
Bacillus subtilis TadA:
MTQDELYMKEAIKEAKKAEEKGEVPIGAVLVINGEIIARAHNLRETEQRS
IAHAEMLVIDEACKALGTWRLEGATLYVTLEPCPMCAGAVVLSRVEKVVF
GAFDPKGGCSGTLMNLLQEERFNHQAEVVSGVLEEECGGMLSAFFRELRK
KKKAARKNLSE
Salmonella typhimurium (S. typhimurium) TadA:
MPPAFITGVTSLSDVELDHEYWMRHALTLAKRAWDEREVPVGAVLVHNHR
VIGEGWNRPIGRHDPTAHAEIMALRQGGLVLQNYRLLDTTLYVTLEPCVM
CAGAMVHSRIGRVVFGARDAKTGAAGSLIDVLHHPGMNHRVEIIEGVLRD
ECATLLSDFFRMRRQEIKALKKADRAEGAGPAV
Shewanella putrefaciens (S. putrefaciens) TadA:
MDEYWMQVAMQMAEKAEAAGEVPVGAVLVKDGQQIATGYNLSISQHDPTA
HAEILCLRSAGKKLENYRLLDATLYITLEPCAMCAGAMVHSRIARVVYGA
RDEKTGAAGTVVNLLQHPAFNHQVEVTSGVLAEACSAQLSRFFKRRRDEK
KALKLAQRAQQGIE
Haemophilus influenzae F3031 (H. influenzae) TadA:
MDAAKVRSEFDEKMMRYALELADKAEALGEIPVGAVLVDDARNIIGEGWN
LSIVQSDPTAHAEIIALRNGAKNIQNYRLLNSTLYVTLEPCTMCAGAILH
SRIKRLVFGASDYKTGAIGSRFHFFDDYKMNHTLEITSGVLAEECSQKLS
TFFQKRREEKKIEKALLKSLSDK
Caulobacter crescentus (C. crescentus) TadA:
MRTDESEDQDHRMMRLALDAARAAAEAGETPVGAVILDPSTGEVIATAGN
GPIAAHDPTAHAEIAAMRAAAAKLGNYRLTDLTLVVTLEPCAMCAGAISH
ARIGRVVFGADDPKGGAVVHGPKFFAQPTCHWRPEVTGGVLADESADLLR
GFFRARRKAKI
Geobacter sulfurreducens (G. sulfurreducens) TadA:
MSSLKKTPIRDDAYWMGKAIREAAKAAARDEVPIGAVIVRDGAVIGRGHN
LREGSNDPSAHAEMIAIRQAARRSANWRLTGATLYVTLEPCLMCMGAIIL
ARLERVVFGCYDPKGGAAGSLYDLSADPRLNHQVRLSPGVCQEECGTMLS
DFFRDLRRRKKAKATPALFIDERKVPPEP

An embodiment of E. Coli TadA (ecTadA) includes the following:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD

In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In one embodiment, a fusion protein of the disclosure comprises a wild-type TadA linked to TadA*7.10, which is linked to Cas9 nickase. In particular embodiments, the fusion proteins comprise a single TadA*7.10 domain (e.g., provided as a monomer). In other embodiments, the ABE7.10 editor comprises TadA*7.10 and TadA(wt), which are capable of forming heterodimers.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

It should be appreciated that any of the mutations provided herein (e.g., based on the TadA reference sequence) can be introduced into other adenosine deaminases, such as E. coli TadA (ecTadA), S. aureus TadA (saTadA), or other adenosine deaminases (e.g., bacterial adenosine deaminases). It would be apparent to the skilled artisan that additional deaminases may similarly be aligned to identify homologous amino acid residues that can be mutated as provided herein. Thus, any of the mutations identified in the TadA reference sequence can be made in other adenosine deaminases (e.g., ecTada) that have homologous amino acid residues. It should also be appreciated that any of the mutations provided herein can be made individually or in any combination in the TadA reference sequence or another adenosine deaminase.

In some embodiments, the adenosine deaminase comprises a D108X mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a D108G, D108N, D108V, D108A, or D108Y mutation, or a corresponding mutation in another adenosine deaminase.

In some embodiments, the adenosine deaminase comprises an A106X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an A106V mutation in TadA reference sequence, or a corresponding mutation in another adenosine deaminase (e.g., wild-type TadA or ecTadA).

In some embodiments, the adenosine deaminase comprises a E155X mutation in TadA reference sequence, or a corresponding mutation in another adenosine deaminase (e.g., ecTadA), where the presence of X indicates any amino acid other than the corresponding amino acid in the wild-type adenosine deaminase. In some embodiments, the adenosine deaminase comprises a E155D, E155G, or E155V mutation in TadA reference sequence, or a corresponding mutation in another adenosine deaminase (e.g., ecTadA).

In some embodiments, the adenosine deaminase comprises a D147X mutation in TadA reference sequence, or a corresponding mutation in another adenosine deaminase (e.g., ecTadA), where the presence of X indicates any amino acid other than the corresponding amino acid in the wild-type adenosine deaminase. In some embodiments, the adenosine deaminase comprises a D147Y, mutation in TadA reference sequence, or a corresponding mutation in another adenosine deaminase (e.g., ecTadA).

In some embodiments, the adenosine deaminase comprises an A106X, E155X, or D147X, mutation in the TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E155D, E155G, or E155V mutation. In some embodiments, the adenosine deaminase comprises a D147Y.

For example, an adenosine deaminase can contain a D108N, a A106V, a E155V, and/or a D147Y mutation in TadA reference sequence, or a corresponding mutation in another adenosine deaminase (e.g., ecTadA). In some embodiments, an adenosine deaminase comprises the following group of mutations (groups of mutations are separated by a “;”) in TadA reference sequence, or corresponding mutations in another adenosine deaminase (e.g., ecTadA): D108N and A106V; D108N and E155V; D108N and D147Y; A106V and E155V; A106V and D147Y; E155V and D147Y; D108N, A106V, and E155V; D108N, A106V, and D147Y; D108N, E155V, and D147Y; A106V, E155V, and D147Y; and D108N, A106V, E155V, and D147Y. It should be appreciated, however, that any combination of corresponding mutations provided herein can be made in an adenosine deaminase (e.g., ecTadA).

In some embodiments, the adenosine deaminase comprises one or more of a H8X, T17X, L18X, W23X, L34X, W45X, R51X, A56X, E59X, E85X, M94X, I95X, V102X, F104X, A106X, R107X, D108X, K110X, M118X, N127X, A138X, F149X, M151X, R153X, Q154X, I156X, and/or K157X mutation in TadA reference sequence, or one or more corresponding mutations in another adenosine deaminase (e.g., ecTadA), where the presence of X indicates any amino acid other than the corresponding amino acid in the wild-type adenosine deaminase. In some embodiments, the adenosine deaminase comprises one or more of H8Y, T17S, L18E, W23L, L34S, W45L, R51H, A56E, or A56S, E59G, E85K, or E85G, M94L, I95L, V102A, F104L, A106V, R107C, or R107H, or R107P, D108G, or D108N, or D108V, or D108A, or D108Y, K110I, M118K, N127S, A138V, F149Y, M151V, R153C, Q154L, I156D, and/or K157R mutation in TadA reference sequence, or one or more corresponding mutations in another adenosine deaminase (e.g., ecTadA).

In some embodiments, the adenosine deaminase comprises one or more of a H8X, D108X, and/or N127X mutation in TadA reference sequence, or one or more corresponding mutations in another adenosine deaminase (e.g., ecTadA), where X indicates the presence of any amino acid. In some embodiments, the adenosine deaminase comprises one or more of a H8Y, D108N, and/or N127S mutation in TadA reference sequence, or one or more corresponding mutations in another adenosine deaminase (e.g., ecTadA).

In some embodiments, the adenosine deaminase comprises one or more of H8X, R26X, M61X, L68X, M70X, A106X, D108X, A109X, N127X, D147X, R152X, Q154X, E155X, K161X, Q163X, and/or T166X mutation in TadA reference sequence, or one or more corresponding mutations in another adenosine deaminase (e.g., ecTadA), where X indicates the presence of any amino acid other than the corresponding amino acid in the wild-type adenosine deaminase. In some embodiments, the adenosine deaminase comprises one or more of H8Y, R26W, M61I, L68Q, M70V, A106T, D108N, A109T, N127S, D147Y, R152C, Q154H or Q154R, E155G or E155V or E155D, K161Q, Q163H, and/or T166P mutation in TadA reference sequence, or one or more corresponding mutations in another adenosine deaminase (e.g., ecTadA).

In some embodiments, the adenosine deaminase comprises one, two, three, four, five, or six mutations selected from the group consisting of H8X, D108X, N127X, D147X, R152X, and Q154X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, M61X, M70X, D108X, N127X, Q154X, E155X, and Q163X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, N127X, E155X, and T166X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8X, A106X, D108X, mutation or mutations in another adenosine deaminase, where X indicates the presence of any amino acid other than the corresponding amino acid in the wild-type adenosine deaminase. In some embodiments, the adenosine deaminase comprises one, two, three, four, five, six, seven, or eight mutations selected from the group consisting of H8X, R26X, L68X, D108X, N127X, D147X, and E155X, or a corresponding mutation or mutations in another adenosine deaminase, where X indicates the presence of any amino acid other than the corresponding amino acid in the wild-type adenosine deaminase. In some embodiments, the adenosine deaminase comprises one, two, three, four, or five, mutations selected from the group consisting of H8X, D108X, A109X, N127X, and E155X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, or six mutations selected from the group consisting of H8Y, D108N, N127S, D147Y, R152C, and Q154H in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, M61I, M70V, D108N, N127S, Q154R, E155G and Q163H in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, N127S, E155V, and T166P in TadA reference sequence, or a corresponding mutation or mutations in another adenosine deaminase (e.g., ecTadA). In some embodiments, the adenosine deaminase comprises one, two, three, four, five, or six mutations selected from the group consisting of H8Y, A106T, D108N, N127S, E155D, and K161Q in TadA reference sequence, or a corresponding mutation or mutations in another adenosine deaminase (e.g., ecTadA). In some embodiments, the adenosine deaminase comprises one, two, three, four, five, six, seven, or eight mutations selected from the group consisting of H8Y, R26W, L68Q, D108N, N127S, D147Y, and E155V in TadA reference sequence, or a corresponding mutation or mutations in another adenosine deaminase (e.g., ecTadA). In some embodiments, the adenosine deaminase comprises one, two, three, four, or five, mutations selected from the group consisting of H8Y, D108N, A109T, N127S, and E155G in TadA reference sequence, or a corresponding mutation or mutations in another adenosine deaminase (e.g., ecTadA).

Any of the mutations provided herein and any additional mutations (e.g., based on the ecTadA amino acid sequence) can be introduced into any other adenosine deaminases. Any of the mutations provided herein can be made individually or in any combination in TadA reference sequence or another adenosine deaminase (e.g., ecTadA).

Details of A to G nucleobase editing proteins are described in International PCT Application No. PCT/2017/045381 (WO2018/027078) and Gaudelli, N. M., et al., “Programmable base editing of A·T to G·C in genomic DNA without DNA cleavage” Nature, 551, 464-471 (2017), the entire contents of which are hereby incorporated by reference.

In some embodiments, the adenosine deaminase comprises one or more corresponding mutations in another adenosine deaminase (e.g., ecTadA). In some embodiments, the adenosine deaminase comprises a D108N, D108G, or D108V mutation in TadA reference sequence, or corresponding mutations in another adenosine deaminase (e.g., ecTadA). In some embodiments, the adenosine deaminase comprises a A106V and D108N mutation in TadA reference sequence, or corresponding mutations in another adenosine deaminase (e.g., ecTadA). In some embodiments, the adenosine deaminase comprises R107C and D108N mutations in TadA reference sequence, or corresponding mutations in another adenosine deaminase (e.g., ecTadA). In some embodiments, the adenosine deaminase comprises a H8Y, D108N, N127S, D147Y, and Q154H mutation in TadA reference sequence, or corresponding mutations in another adenosine deaminase (e.g., ecTadA). In some embodiments, the adenosine deaminase comprises a H8Y, D108N, N127S, D147Y, and E155V mutation in TadA reference sequence, or corresponding mutations in another adenosine deaminase (e.g., ecTadA). In some embodiments, the adenosine deaminase comprises a D108N, D147Y, and E155V mutation in TadA reference sequence, or corresponding mutations in another adenosine deaminase (e.g., ecTadA). In some embodiments, the adenosine deaminase comprises a H8Y, D108N, and N127S mutation in TadA reference sequence, or corresponding mutations in another adenosine deaminase (e.g., ecTadA). In some embodiments, the adenosine deaminase comprises a A106V, D108N, D147Y and E155V mutation in TadA reference sequence, or corresponding mutations in another adenosine deaminase (e.g., ecTadA).

In some embodiments, the adenosine deaminase comprises one or more of a S2X, H8X, I49X, L84X, H123X, N127X, I156X and/or K160X mutation in TadA reference sequence, or one or more corresponding mutations in another adenosine deaminase, where the presence of X indicates any amino acid other than the corresponding amino acid in the wild-type adenosine deaminase. In some embodiments, the adenosine deaminase comprises one or more of S2A, H8Y, I49F, L84F, H123Y, N127S, I156F and/or K160S mutation in TadA reference sequence, or one or more corresponding mutations in another adenosine deaminase (e.g., ecTadA).

In some embodiments, the adenosine deaminase comprises an L84X mutation adenosine deaminase, where X indicates any amino acid other than the corresponding amino acid in the wild-type adenosine deaminase. In some embodiments, the adenosine deaminase comprises an L84F mutation in TadA reference sequence, or a corresponding mutation in another adenosine deaminase (e.g., ecTadA).

In some embodiments, the adenosine deaminase comprises an H123X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an H123Y mutation in TadA reference sequence, or a corresponding mutation in another adenosine deaminase (e.g., ecTadA).

In some embodiments, the adenosine deaminase comprises an I156X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an I156F mutation in TadA reference sequence, or a corresponding mutation in another adenosine deaminase (e.g., ecTadA).

In some embodiments, the adenosine deaminase comprises one, two, three, four, five, six, or seven mutations selected from the group consisting of L84X, A106X, D108X, H123X, D147X, E155X, and I156X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, five, or six mutations selected from the group consisting of S2X, I49X, A106X, D108X, D147X, and E155X in TadA reference sequence, or a corresponding mutation or mutations in another adenosine deaminase (e.g., ecTadA), where X indicates the presence of any amino acid other than the corresponding amino acid in the wild-type adenosine deaminase. In some embodiments, the adenosine deaminase comprises one, two, three, four, or five, mutations selected from the group consisting of H8X, A106X, D108X, N127X, and K160X in TadA reference sequence, or a corresponding mutation or mutations in another adenosine deaminase (e.g., ecTadA), where X indicates the presence of any amino acid other than the corresponding amino acid in the wild-type adenosine deaminase.

In some embodiments, the adenosine deaminase comprises one, two, three, four, five, six, or seven mutations selected from the group consisting of L84F, A106V, D108N, H123Y, D147Y, E155V, and I156F in TadA reference sequence, or a corresponding mutation or mutations in another adenosine deaminase (e.g., ecTadA). In some embodiments, the adenosine deaminase comprises one, two, three, four, five, or six mutations selected from the group consisting of S2A, I49F, A106V, D108N, D147Y, and E155V in TadA reference sequence.

In some embodiments, the adenosine deaminase comprises one, two, three, four, or five, mutations selected from the group consisting of H8Y, A106T, D108N, N127S, and K160S in TadA reference sequence, or a corresponding mutation or mutations in another adenosine deaminase (e.g., ecTadA).

In some embodiments, the adenosine deaminase comprises one or more of a E25X, R26X, R107X, A142X, and/or A143X mutation in TadA reference sequence, or one or more corresponding mutations in another adenosine deaminase (e.g., ecTadA), where the presence of X indicates any amino acid other than the corresponding amino acid in the wild-type adenosine deaminase. In some embodiments, the adenosine deaminase comprises one or more of E25M, E25D, E25A, E25R, E25V, E25S, E25Y, R26G, R26N, R26Q, R26C, R26L, R26K, R107P, R107K, R107A, R107N, R107W, R107H, R107S, A142N, A142D, A142G, A143D, A143G, A143E, A143L, A143W, A143M, A143S, A143Q and/or A143R mutation in TadA reference sequence, or one or more corresponding mutations in another adenosine deaminase (e.g., ecTadA). In some embodiments, the adenosine deaminase comprises one or more of the mutations described herein corresponding to TadA reference sequence, or one or more corresponding mutations in another adenosine deaminase (e.g., ecTadA).

In some embodiments, the adenosine deaminase comprises an E25X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an E25M, E25D, E25A, E25R, E25V, E25S, or E25Y mutation in TadA reference sequence, or a corresponding mutation in another adenosine deaminase (e.g., ecTadA).

In some embodiments, the adenosine deaminase comprises an R26X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises R26G, R26N, R26Q, R26C, R26L, or R26K mutation in TadA reference sequence, or a corresponding mutation in another adenosine deaminase (e.g., ecTadA).

In some embodiments, the adenosine deaminase comprises an R107X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an R107P, R107K, R107A, R107N, R107W, R107H, or R107S mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an A142N, A142D, A142G, mutation in TadA reference sequence, or a corresponding mutation in another adenosine deaminase (e.g., ecTadA).

In some embodiments, the adenosine deaminase comprises an A143X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an A143D, A143G, A143E, A143L, A143W, A143M, A143S, A143Q and/or A143R mutation in TadA reference sequence, or a corresponding mutation in another adenosine deaminase (e.g., ecTadA).

In some embodiments, the adenosine deaminase comprises one or more of a H36X, N37X, P48X, I49X, R51X, M70X, N72X, D77X, E134X, S146X, Q154X, K157X, and/or K161X mutation in TadA reference sequence, or one or more corresponding mutations in another adenosine deaminase (e.g., ecTadA), where the presence of X indicates any amino acid other than the corresponding amino acid in the wild-type adenosine deaminase. In some embodiments, the adenosine deaminase comprises one or more of H36L, N37T, N37S, P48T, P48L, I49V, R51H, R51L, M70L, N72S, D77G, E134G, S146R, S146C, Q154H, K157N, and/or K161T mutation in TadA reference sequence, or one or more corresponding mutations in another adenosine deaminase (e.g., ecTadA).

In some embodiments, the adenosine deaminase comprises an H36X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an H36L mutation in TadA reference sequence, or a corresponding mutation in another adenosine deaminase (e.g., ecTadA).

In some embodiments, the adenosine deaminase comprises an N37X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an N37T, or N37S mutation in TadA reference sequence, or a corresponding mutation in another adenosine deaminase (e.g., ecTadA).

In some embodiments, the adenosine deaminase comprises an P48X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an P48T, or P48L mutation in TadA reference sequence, or a corresponding mutation in another adenosine deaminase (e.g., ecTadA).

In some embodiments, the adenosine deaminase comprises an R51X mutation in TadA reference sequence, or a corresponding mutation in another adenosine deaminase, where X indicates any amino acid other than the corresponding amino acid in the wild-type adenosine deaminase. In some embodiments, the adenosine deaminase comprises an R51H, or R51L mutation in TadA reference sequence, or a corresponding mutation in another adenosine deaminase (e.g., ecTadA).

In some embodiments, the adenosine deaminase comprises an S146X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises an S146R, or S146C mutation in TadA reference sequence, or a corresponding mutation in another adenosine deaminase (e.g., ecTadA).

In some embodiments, the adenosine deaminase comprises an K157X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a K157N mutation in TadA reference sequence, or a corresponding mutation in another adenosine deaminase (e.g., ecTadA).

In some embodiments, the adenosine deaminase comprises an P48X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a P48S, P48T, or P48A mutation in TadA reference sequence, or a corresponding mutation in another adenosine deaminase (e.g., ecTadA).

In some embodiments, the adenosine deaminase comprises an A142X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a A142N mutation in TadA reference sequence, or a corresponding mutation in another adenosine deaminase (e.g., ecTadA).

In some embodiments, the adenosine deaminase comprises an W23X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a W23R, or W23L mutation in TadA reference sequence, or a corresponding mutation in another adenosine deaminase (e.g., ecTadA).

In some embodiments, the adenosine deaminase comprises an R152X mutation in TadA reference sequence, or a corresponding mutation in another adenosine deaminase (e.g., ecTadA), where X indicates any amino acid other than the corresponding amino acid in the wild-type adenosine deaminase. In some embodiments, the adenosine deaminase comprises a R152P, or R52H mutation in TadA reference sequence, or a corresponding mutation in another adenosine deaminase (e.g., ecTadA).

In one embodiment, the adenosine deaminase may comprise the mutations H36L, R51L, L84F, A106V, D108N, H123Y, S146C, D147Y, E155V, I156F, and K157N. In some embodiments, the adenosine deaminase comprises the following combination of mutations relative to TadA reference sequence, where each mutation of a combination is separated by a “_” and each combination of mutations is between parentheses:

### (A106V_D108N),

### (R107C_D108N),

### (H8Y_D108N_N127S_D147Y_Q154H),

### (H8Y_D108N_N127S_D147Y_E155V),

### (D108N_D147Y_E155V),

### (H8Y_D108N_N127S),

### (H8Y_D108N_N127S_D147Y_Q154H),

### (A106V_D108N_D147Y_E155V),

### (D108Q_D147Y_E155V),

### (D108M_D147Y_E155V),

### (D108L_D147Y_E155V),

### (D108K_D147Y_E155V),

### (D108I_D147Y_E155V),

### (D108F_D147Y_E155V),

### (A106V_D108N_D147Y),

### (A106V_D108M_D147Y_E155V),

### (E59A_A106V_D108N_D147Y_E155V),

(E59A cat dead_A106V_D108N_D147Y_E155V),

### (L84F_A106V_D108N_H123Y_D147Y_E155V_I156Y),

### (L84F_A106V_D108N_H123Y_D147Y_E155V_156F),

### (D103A_D104N),

### (G22P_D103A_D104N),

### (D103A_D104N_S138A),

### (R26G_L84F_A106V_R107H_D108N_H123Y_A142N_A143D_D147Y_E155V_I156F),

### (E25G_R26G_L84F_A106V_R107H_D108N_H123Y_A142N_A143D_D147Y_E155V_1156F),

### (E25D_R26G_L84F_A106V_R107K_D108N_H123Y_A142N_A143G_D147Y_E155V_I156F),

### (R26Q_L84F_A106V_D108N_H123Y_A142N_D147Y_E155V_I156F),

### (E25M_R26G_L84F_A106V_R107P_D108N_H123Y_A142N_A143D_D147Y_E155V_1156F),

### (R26C_L84F_A106V_R107H_D108N_H123Y_A142N_D147Y_E155V_I156F),

### (L84F_A106V_D108N_H123Y_A142N_A143L_D147Y_E155V_I156F),

### (R26G_L84F_A106V_D108N_H123Y_A142N_D147Y_E155V_I156F),

### (E25A_R26G_L84F_A106V_R107N_D108N_H123Y_A142N_A143E_D147Y_E155V_1156F),

### (R26G_L84F_A106V_R107H_D108N_H123Y_A142N_A143D_D147Y_E155V_I156F),

### (A106V_D108N_A142N_D147Y_E155V),

### (R26G_A106V_D108N_A142N_D147Y_E155V),

### (E25D_R26G_A106V_R107K_D108N_A142N_A143G_D147Y_E155V),

### (R26G_A106V_D108N_R107H_A142N_A143D_D147Y_E155V),

### (E25D_R26G_A106V_D108N_A142N_D147Y_E155V),

### (A106V_R107K_D108N_A142N_D147Y_E155V),

### (A106V_D108N_A142N_A143G_D147Y_E155V),

### (A106V_D108N_A142N_A143L_D147Y_E155V),

### (H36L_R51L_L84F_A106V_D108N_H123Y_S146C_D147Y_E155V_I156F_K157N),

### (N37T_P48T_M70L_L84F_A106V_D108N_H123Y_D147Y_I49V_E155V_I156F),

### (N37S_L84F_A106V_D108N_H123Y_D147Y_E155V_156F_K161T),

### (H36L_L84F_A106V_D108N_H123Y_D147Y_Q154H_E155V_I156F),

### (N72S_L84F_A106V_D108N_H123Y_S146R_D147Y_E155V_I156F),

### (H36L_P48L_L84F_A106V_D108N_H123Y_E134G_D147Y_E155V_I156F),

### (H36L_L84F_A106V_D108N_H123Y_D147Y_E155V_I156F_K157N),

### (H36L_L84F_A106V_D108N_H123Y_S146C_D147Y_E155V_I156F),

### (L84F_A106V_D108N_H123Y_S146R_D147Y_E155V_I156F_K161T),

### (N37S_R51H_D77G_L84F_A106V_D108N_H123Y_D147Y_E155V_I156F),

### (R51L_L84F_A106V_D108N_H123Y_D147Y_E155V_I156F_K157N),

### (D24G_Q71R_L84F_H96L_A106V_D108N_H123Y_D147Y_E155V_I156F_K160E),

### (H36L_G67V_L84F_A106V_D108N_H123Y_S146T_D147Y_E155V_I156F),

### (Q71L_L84F_A106V_D108N_H123Y_L137M_A143E_D147Y_E155V_I156F),

### (E25G_L84F_A106V_D108N_H123Y_D147Y_E155V_I156F_Q159L),

### (L84F_A91T_F104I_A106V_D108N_H123Y_D147Y_E155V_I156F),

### (N72D_L84F_A106V_D108N_H123Y_G125A_D147Y_E155V_I156F),

### (P48S_L84F_S97C_A106V_D108N_H123Y_D147Y_E155V_I156F),

### (W23G_L84F_A106V_D108N_H123Y_D147Y_E155V_I156F),

### (D24G_P48L_Q71R_L84F_A106V_D108N_H123Y_D147Y_E155V_I156F_Q159L),

### (L84F_A106V_D108N_H123Y_A142N_D147Y_E155V_I156F),

### (H36L_R51L_L84F_A106V_D108N_H123Y_A142N_S146C_D147Y_E155V_I156F_K157N), (N37S_L84F_A106V_D108N_H123Y_A142N_D147Y_E155V_I156F_K161T),

### (L84F_A106V_D108N_D147Y_E155V_I156F),

### (R51L_L84F_A106V_D108N_H123Y_S146C_D147Y_E155V_I156F_K157N_K161T),

### (L84F_A106V_D108N_H123Y_S146C_D147Y_E155V_156F_K161T),

### (L84F_A106V_D108N_H123Y_S146C_D147Y_E155V_I156F_K157N_K160E_K161T),

### (L84F_A106V_D108N_H123Y_S146C_D147Y_E155V_I156F_K157N_K160E),

### (R74Q_L84F_A106V_D108N_H123Y_D147Y_E155V_I156F),

### (R74A_L84F_A106V_D108N_H123Y_D147Y_E155V_I156F),

### (L84F_A106V_D108N_H123Y_D147Y_E155V_156F),

### (R74Q_L84F_A106V_D108N_H123Y_D147Y_E155V_I156F),

### (L84F_R98Q_A106V_D108N_H123Y_D147Y_E155V_I156F),

### (L84F_A106V_D108N_H123Y_R129Q_D147Y_E155V_I156F),

### (P48S_L84F_A106V_D108N_H123Y_A142N_D147Y_E155V_I156F),

### (P48S_A142N),

### (P48T_I49V_L84F_A106V_D108N_H123Y_A142N_D147Y_E155V_I156F_L157N),

### (P48T_I49V_A142N),

### (H36L_P48S_R51L_L84F_A106V_D108N_H123Y_S146C_D147Y_E155V_I156F_K157N),

### (H36L_P48S_R51L_L84F_A106V_D108N_H123Y_S146C_A142N_D147Y_E155V_I156F

### (H36L_P48T_I49V_R51L_L84F_A106V_D108N_H123Y_S146C_D147Y_E155V_I156F_K157N),

### (H36L_P48T_I49V_R51L_L84F_A106V_D108N_H123Y_A142N_S146C_D147Y_E155V_I156F_K157N),

### (H36L_P48A_R51L_L84F_A106V_D108N_H123Y_S146C_D147Y_E155V_I156F_K157N),

### (H36L_P48A_R51L_L84F_A106V_D108N_H123Y_A142N_S146C_D147Y_E155V_I156F_K157N),

### (H36L_P48A_R51L_L84F_A106V_D108N_H123Y_S146C_A142N_D147Y_E155V_I156F_K157N),

### (W23L_H36L_P48A_R51L_L84F_A106V_D108N_H123Y_S146C_D147Y_E155V_I156F_K157N),

### (W23R_H36L_P48A_R51L_L84F_A106V_D108N_H123Y_S146C_D147Y_E155V_I156F_K157N),

### (W23L_H36L_P48A_R51L_L84F_A106V_D108N_H123Y_S146R_D147Y_E155V_I156F_K161T),

### (H36L_P48A_R51L_L84F_A106V_D108N_H123Y_S146C_D147Y_R152H_E155V_I156F_K157N),

### (H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_S146C_D147Y_R152P_E155V_I156F_K157N),

### (W23L_H36L_P48A_R51L_L84F_A106V_D108N_H123Y_A142A_S146C_D147Y_E155 V_I156F_K157N),

### (W23L_H36L_P48A_R51L_L84F_A106V_D108N_H123Y_A142A_S146C_D147Y_R152 P_E155V_I156F_K157N),

### (W23L_H36L_P48A_R51L_L84F_A106V_D108N_H123Y_S146R_D147Y_E155V_I156F_K161T),

### (W23R_H36L_P48A_R51L_L84F_A106V_D108N_H123Y_S146C_D147Y_R152P_E155V_I156F_K157N),

### (H36L_P48A_R51L_L84F_A106V_D108N_H123Y_A142N_S146C_D147Y_R152P_E155 V_I156F_K157N).

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9).

In some embodiments, the adenosine deaminase is TadA*7.10. In some embodiments, TadA*7.10 comprises at least one alteration. In particular embodiments, TadA*7.10 comprises one or more of the following alterations: Y147T, Y147R, Q154S, Y123H, V82S, T166R, and Q154R. The alteration Y123H is also referred to herein as H123H (the alteration H123Y in TadA*7.10 reverted back to Y123H (wt)). In other embodiments, the TadA*7.10 comprises a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+176Y; V82S+Y123H+Y147R+Q154R; and I76Y+V82S+Y123H+Y147R+Q154R. In particular embodiments, an adenosine deaminase variant comprises a deletion of the C terminus beginning at residue 149, 150, 151, 152, 153, 154, 155, 156, and 157, relative to TadA*7.10, the TadA reference sequence, or a corresponding mutation in another TadA.

In other embodiments, a base editor of the disclosure is a monomer comprising an adenosine deaminase variant (e.g., TadA*8) comprising one or more of the following alterations: Y147T, Y147R, Q154S, Y123H, V82S, T166R, and/or Q154R, relative to TadA*7.10, the TadA reference sequence, or a corresponding mutation in another TadA. In other embodiments, the adenosine deaminase variant (TadA*8) is a monomer comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA. In other embodiments, a base editor is a heterodimer comprising a wild-type adenosine deaminase and an adenosine deaminase variant (e.g., TadA*8) comprising one or more of the following alterations Y147T, Y147R, Q154S, Y123H, V82S, T166R, and/or Q154R, relative to TadA*7.10, the TadA reference sequence, or a corresponding mutation in another TadA. In other embodiments, the base editor is a heterodimer comprising a TadA*7.10 domain and an adenosine deaminase variant domain (e.g., TadA*8) comprising a combination of alterations selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+I76Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In one embodiment, an adenosine deaminase is a TadA*8 that comprises or consists essentially of the following sequence or a fragment thereof having adenosine deaminase activity:

MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSTD

In some embodiments, the TadA*8 is a truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In some embodiments the TadA*8 is TadA*8.1, TadA*8.2, TadA*8.3, TadA*8.4, TadA*8.5, TadA*8.6, TadA*8.7, TadA*8.8, TadA*8.9, TadA*8.10, TadA*8.11, TadA*8.12, TadA*8.13, TadA*8.14, TadA*8.15, TadA*8.16, TadA*8.17, TadA*8.18, TadA*8.19, TadA*8.20, TadA*8.21, TadA*8.22, TadA*8.23, or TadA*8.24.

In one embodiment, a fusion protein of the disclosure comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers. Exemplary sequences follow:

TadA(wt) or “the TadA reference sequence”:
MSEVEFSHEYWMRHALTLAKRAWDEREVPVGAVLVHNNRVIGEGWNRPIG
RHDPTAHAEIMALRQGGLVMQNYRLIDATLYVTLEPCVMCAGAMIHSRIG
RVVFGARDAKTGAAGSLMDVLHHPGMNHRVEITEGILADECAALLSDFFR
MRRQEIKAQKKAQSSTD
TadA*7.10:
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCYFFR
MPRQVFNAQKKAQSSTD
TadA*8:
MSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIG
RVVFGVRNAKTGAAGSLMDVLHYPGMNHRVEITEGILADECAALLCTFFR
MPRQVFNAQKKAQSSID.

In some embodiments, the adenosine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the amino acid sequences set forth in any of the adenosine deaminases provided herein. It should be appreciated that adenosine deaminases provided herein may include one or more mutations (e.g., any of the mutations provided herein). The disclosure provides any deaminase domains with a certain percent identity plus any of the mutations or combinations thereof described herein. In some embodiments, the adenosine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the adenosine deaminases provided herein. In some embodiments, the adenosine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

In particular embodiments, a TadA*8 comprises one or more mutations at any of the following positions shown in bold. In other embodiments, a TadA*8 comprises one or more mutations at any of the positions shown with underlining:

| | |
|---|---|
| MSEVEFSHEY WMRHALTLAK RARDEREVPV GAVLVLNNRV IGEGWNRAIG | 50 |
| LHDPTAHAEI MALRQGGLVM QNYRLIDATL YVTFEPCVMC AGAMIHSRIG | 100 |
| RVVFGVRNAK TGAAGSLMDV LHYPGMNHRV EITEGILADE CAALLCYFFR | 150 |
| MPRQVFNAQK KAQSSTD | |

For example, the TadA*8 comprises alterations at amino acid position 82 and/or 166 (e.g., V82S, T166R) alone or in combination with any one or more of the following Y147T, Y147R, Q154S, Y123H, and/or Q154R, relative to TadA*7.10, the TadA reference sequence, or a corresponding mutation in another TadA. In particular embodiments, a combination of alterations is selected from the group of: Y147T+Q154R; Y147T+Q154S; Y147R+Q154S; V82S+Q154S; V82S+Y147R; V82S+Q154R; V82S+Y123H; I76Y+V82S; V82S+Y123H+Y147T; V82S+Y123H+Y147R; V82S+Y123H+Q154R; Y147R+Q154R+Y123H; Y147R+Q154R+176Y; Y147R+Q154R+T166R; Y123H+Y147R+Q154R+I76Y; V82S+Y123H+Y147R+Q154R; and I76Y+V82S+Y123H+Y147R+Q154R, relative to TadA*7.10, the TadA reference sequence, or a corresponding mutation in another TadA.

In some embodiments, the adenosine deaminase is TadA*8, which comprises or consists essentially of the following sequence or a fragment thereof having adenosine deaminase activity

MSEVEFSHEY WMRHALTLAK RARDEREVPV GAVLVLNNRV
IGEGWNRAIG LHDPTAHAEI MALRQGGLVM QNYRLIDATL
YVTFEPCVMC AGAMIHSRIG RVVFGVRNAK TGAAGSLMDV
LHYPGMNHRV EITEGILADE CAALLCTFFR MPRQVFNAQK
KAQSSTD

In some embodiments, the TadA*8 is truncated. In some embodiments, the truncated TadA*8 is missing 1, 2, 3, 4, 5, 6, 7, 8, 9, 10, 11, 12, 13, 14, 15, 6, 17, 18, 19, or 20 N-terminal amino acid residues relative to the full length TadA*8. In some embodiments, the truncated TadA*8 is missing 1, 2, 3, 4, 5, 6, 7, 8, 9, 10, 11, 12, 13, 14, 15, 6, 17, 18, 19, or 20 C-terminal amino acid residues relative to the full length TadA*8. In some embodiments the adenosine deaminase variant is a full-length TadA*8.

In one embodiment, a fusion protein of the disclosure comprises a wild-type TadA is linked to an adenosine deaminase variant described herein (e.g., TadA*8), which is linked to Cas9 nickase. In particular embodiments, the fusion proteins comprise a single TadA*8 domain (e.g., provided as a monomer). In other embodiments, the base editor comprises TadA*8 and TadA(wt), which are capable of forming heterodimers.

### C to T Editing

In some embodiments, a base editor disclosed herein comprises a fusion protein comprising cytidine deaminase capable of deaminating a target cytidine (C) base of a polynucleotide to produce uridine (U), which has the base pairing properties of thymine. In some embodiments, for example where the polynucleotide is double-stranded (e.g., DNA), the uridine base can then be substituted with a thymidine base (e.g., by cellular repair machinery) to give rise to a C:G to a T:A transition. In other embodiments, deamination of a C to U in a nucleic acid by a base editor cannot be accompanied by substitution of the U to a T.

The deamination of a target C in a polynucleotide to give rise to a U is a non-limiting example of a type of base editing that can be executed by a base editor described herein. In another example, a base editor comprising a cytidine deaminase domain can mediate conversion of a cytosine (C) base to a guanine (G) base. For example, a U of a polynucleotide produced by deamination of a cytidine by a cytidine deaminase domain of a base editor can be excised from the polynucleotide by a base excision repair mechanism (e.g., by a uracil DNA glycosylase (UDG) domain), producing an abasic site. The nucleobase opposite the abasic site can then be substituted (e.g., by base repair machinery) with another base, such as a C, by for example a translesion polymerase. Although it is typical for a nucleobase opposite an abasic site to be replaced with a C, other substitutions (e.g., A, G or T) can also occur.

Accordingly, in some embodiments a base editor described herein comprises a deamination domain (e.g., cytidine deaminase domain) capable of deaminating a target C to a U in a polynucleotide. Further, as described below, the base editor can comprise additional domains which facilitate conversion of the U resulting from deamination to, in some embodiments, a T or a G. For example, a base editor comprising a cytidine deaminase domain can further comprise a uracil glycosylase inhibitor (UGI) domain to mediate substitution of a U by a T, completing a C-to-T base editing event. In another example, a base editor can incorporate a translesion polymerase to improve the efficiency of C-to-G base editing, since a translesion polymerase can facilitate incorporation of a C opposite an abasic site (i.e., resulting in incorporation of a G at the abasic site, completing the C-to-G base editing event).

A base editor comprising a cytidine deaminase as a domain can deaminate a target C in any polynucleotide, including DNA, RNA and DNA-RNA hybrids. Typically, a cytidine deaminase catalyzes a C nucleobase that is positioned in the context of a single-stranded portion of a polynucleotide. In some embodiments, the entire polynucleotide comprising a target C can be single-stranded. For example, a cytidine deaminase incorporated into the base editor can deaminate a target C in a single-stranded RNA polynucleotide. In other embodiments, a base editor comprising a cytidine deaminase domain can act on a double-stranded polynucleotide, but the target C can be positioned in a portion of the polynucleotide which at the time of the deamination reaction is in a single-stranded state. For example, in embodiments where the NAGPB domain comprises a Cas9 domain, several nucleotides can be left unpaired during formation of the Cas9-gRNA-target DNA complex, resulting in formation of a Cas9 “R-loop complex”. These unpaired nucleotides can form a bubble of single-stranded DNA that can serve as a substrate for a single-strand specific nucleotide deaminase enzyme (e.g., cytidine deaminase).

In some embodiments, a cytidine deaminase of a base editor can comprise all or a portion of an apolipoprotein B mRNA editing complex (APOBEC) family deaminase. APOBEC is a family of evolutionarily conserved cytidine deaminases. Members of this family are C-to-U editing enzymes. The N-terminal domain of APOBEC like proteins is the catalytic domain, while the C-terminal domain is a pseudocatalytic domain. More specifically, the catalytic domain is a zinc dependent cytidine deaminase domain and is important for cytidine deamination. APOBEC family members include APOBEC1, APOBEC2, APOBEC3A, APOBEC3B, APOBEC3C, APOBEC3D (“APOBEC3E” now refers to this), APOBEC3F, APOBEC3G, APOBEC3H, APOBEC4, and Activation-induced (cytidine) deaminase. In some embodiments, a deaminase incorporated into a base editor comprises all or a portion of an APOBEC1 deaminase. In some embodiments, a deaminase incorporated into a base editor comprises all or a portion of APOBEC2 deaminase. In some embodiments, a deaminase incorporated into a base editor comprises all or a portion of is an APOBEC3 deaminase. In some embodiments, a deaminase incorporated into a base editor comprises all or a portion of an APOBEC3A deaminase. In some embodiments, a deaminase incorporated into a base editor comprises all or a portion of APOBEC3B deaminase. In some embodiments, a deaminase incorporated into a base editor comprises all or a portion of APOBEC3C deaminase. In some embodiments, a deaminase incorporated into a base editor comprises all or a portion of APOBEC3D deaminase. In some embodiments, a deaminase incorporated into a base editor comprises all or a portion of APOBEC3E deaminase. In some embodiments, a deaminase incorporated into a base editor comprises all or a portion of APOBEC3F deaminase. In some embodiments, a deaminase incorporated into a base editor comprises all or a portion of APOBEC3G deaminase. In some embodiments, a deaminase incorporated into a base editor comprises all or a portion of APOBEC3H deaminase. In some embodiments, a deaminase incorporated into a base editor comprises all or a portion of APOBEC4 deaminase. In some embodiments, a deaminase incorporated into a base editor comprises all or a portion of activation-induced deaminase (AID). In some embodiments a deaminase incorporated into a base editor comprises all or a portion of cytidine deaminase 1 (CDA1). It should be appreciated that a base editor can comprise a deaminase from any suitable organism (e.g., a human or a rat). In some embodiments, a deaminase domain of a base editor is from a human, chimpanzee, gorilla, monkey, cow, dog, rat, or mouse. In some embodiments, the deaminase domain of the base editor is derived from rat (e.g., rat APOBEC1). In some embodiments, the deaminase domain of the base editor is human APOBEC1. In some embodiments, the deaminase domain of the base editor is pmCDA1.

The amino acid and nucleic acid sequences of PmCDA1 are shown herein below.

>tr|A5H718|A5H718_PETMA Cytosine deaminase OS=Petromyzon marinus OX=7757 PE=2 SV=1 amino acid sequence:

MTDAEYVRIHEKLDIYTFKKQFFNNKKSVSHRCYVLFELKRRGERRACFW
GYAVNKPQSGTERGIHAEIFSIRKVEEYLRDNPGQFTINWYSSWSPCADC
AEKILEWYNQELRGNGHTLKIWACKLYYEKNARNQIGLWNLRDNGVGLNV
MVSEHYQCCRKIFIQSSHNQLNENRWLEKTLKRAEKRRSELSIMIQVKIL
HTTKSPAV

Nucleic acid sequence: >EF094822.1 Petromyzon marinus isolate PmCDA.21 cytosine deaminase mRNA, complete cds:

TGACACGACACAGCCGTGTATATGAGGAAGGGTAGCTGGATGGGGGGGGG
GGGAATACGTTCAGAGAGGACATTAGCGAGCGTCTTGTTGGTGGCCTTGA
GTCTAGACACCTGCAGACATGACCGACGCTGAGTACGTGAGAATCCATGA
GAAGTTGGACATCTACACGTTTAAGAAACAGTTTTTCAACAACAAAAAAT
CCGTGTCGCATAGATGCTACGTTCTCTTTGAATTAAAACGACGGGGTGAA
CGTAGAGCGTGTTTTTGGGGCTATGCTGTGAATAAACCACAGAGCGGGAC
AGAACGTGGAATTCACGCCGAAATCTTTAGCATTAGAAAAGTCGAAGAAT
ACCTGCGCGACAACCCCGGACAATTCACGATAAATTGGTACTCATCCTGG
AGTCCTTGTGCAGATTGCGCTGAAAAGATCTTAGAATGGTATAACCAGGA
GCTGCGGGGGAACGGCCACACTTTGAAAATCTGGGCTTGCAAACTCTATT
ACGAGAAAAATGCGAGGAATCAAATTGGGCTGTGGAACCTCAGAGATAAC
GGGGTTGGGTTGAATGTAATGGTAAGTGAACACTACCAATGTTGCAGGAA
AATATTCATCCAATCGTCGCACAATCAATTGAATGAGAATAGATGGCTTG
AGAAGACTTTGAAGCGAGCTGAAAAACGACGGAGCGAGTTGTCCATTATG
ATTCAGGTAAAAATACTCCACACCACTAAGAGTCCTGCTGTTTAAGAGGC
TATGCGGATGGTTTTC

The amino acid and nucleic acid sequences of the coding sequence (CDS) of human activation-induced cytidine deaminase (AID) are shown below.

>tr|Q6QJ80|Q6QJ80_HUMAN Activation-induced cytidine deaminase OS═Homo sapiens OX=9606 GN=AICDA PE=2 SV=1 amino acid sequence:

MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKAPV

The amino acid and nucleic acid sequences of the coding sequence (CDS) of human activation-induced cytidine deaminase (AID) are shown below.

>tr|Q6QJ80|Q6QJ80_HUMAN Activation-induced cytidine deaminase OS═Homo sapiens OX=9606 GN=AICDA PE=2 SV=1 amino acid sequence:

MDSLLMNRRKFLYQFKNVRWAKGRRETYLCYVVKRRDSATSFSLDFGYLR
NKNGCHVELLFLRYISDWDLDPGRCYRVTWFTSWSPCYDCARHVADFLRG
NPNLSLRIFTARLYFCEDRKAEPEGLRRLHRAGVQIAIMTFKAPV

Nucleic acid sequence: >NG_011588.1:5001-15681 Homo sapiens activation induced cytidine deaminase (AICDA), RefSeqGene (LRG_17) on chromosome 12:

AGAGAACCATCATTAATTGAAGTGAGATTTTTCTGGCCTGAGACTTGCAGGGAGGCAAGAAG
ACACTCTGGACACCACTATGGACAGGTAAAGAGGCAGTCTTCTCGTGGGTGATTGCACTGGC
CTTCCTCTCAGAGCAAATCTGAGTAATGAGACTGGTAGCTATCCCTTTCTCTCATGTAACTG
TCTGACTGATAAGATCAGCTTGATCAATATGCATATATATTTTTTGATCTGTCTCCTTTTCT
TCTATTCAGATCTTATACGCTGTCAGCCCAATTCTTTCTGTTTCAGACTTCTCTTGATTTCC
CTCTTTTTCATGTGGCAAAAGAAGTAGTGCGTACAATGTACTGATTCGTCCTGAGATTTGTA
CCATGGTTGAAACTAATTTATGGTAATAATATTAACATAGCAAATCTTTAGAGACTCAAATC
ATGAAAAGGTAATAGCAGTACTGTACTAAAAACGGTAGTGCTAATTTTCGTAATAATTTTGT
AAATATTCAACAGTAAAACAACTTGAAGACACACTTTCCTAGGGAGGCGTTACTGAAATAAT
TTAGCTATAGTAAGAAAATTTGTAATTTTAGAAATGCCAAGCATTCTAAATTAATTGCTTGA
AAGTCACTATGATTGTGTCCATTATAAGGAGACAAATTCATTCAAGCAAGTTATTTAATGTT
AAAGGCCCAATTGTTAGGCAGTTAATGGCACTTTTACTATTAACTAATCTTTCCATTTGTTC
AGACGTAGCTTAACTTACCTCTTAGGTGTGAATTTGGTTAAGGTCCTCATAATGTCTTTATG
TGCAGTTTTTGATAGGTTATTGTCATAGAACTTATTCTATTCCTACATTTATGATTACTATG
GATGTATGAGAATAACACCTAATCCTTATACTTTACCTCAATTTAACTCCTTTATAAAGAAC
TTACATTACAGAATAAAGATTTTTTAAAAATATATTTTTTTGTAGAGACAGGGTCTTAGCCC
AGCCGAGGCTGGTCTCTAAGTCCTGGCCCAAGCGATCCTCCTGCCTGGGCCTCCTAAAGTGC
TGGAATTATAGACATGAGCCATCACATCCAATATACAGAATAAAGATTTTTAATGGAGGATT
TAATGTTCTTCAGAAAATTTTCTTGAGGTCAGACAATGTCAAATGTCTCCTCAGTTTACACT
GAGATTTTGAAAACAAGTCTGAGCTATAGGTCCTTGTGAAGGGTCCATTGGAAATACTTGTT
CAAAGTAAAATGGAAAGCAAAGGTAAAATCAGCAGTTGAAATTCAGAGAAAGACAGAAAAGG
AGAAAAGATGAAATTCAACAGGACAGAAGGGAAATATATTATCATTAAGGAGGACAGTATCT
GTAGAGCTCATTAGTGATGGCAAAATGACTTGGTCAGGATTATTTTTAACCCGCTTGTTTCT
GGTTTGCACGGCTGGGGATGCAGCTAGGGTTCTGCCTCAGGGAGCACAGCTGTCCAGAGCAG
CTGTCAGCCTGCAAGCCTGAAACACTCCCTCGGTAAAGTCCTTCCTACTCAGGACAGAAATG
ACGAGAACAGGGAGCTGGAAACAGGCCCCTAACCAGAGAAGGGAAGTAATGGATCAACAAAG
TTAACTAGCAGGTCAGGATCACGCAATTCATTTCACTCTGACTGGTAACATGTGACAGAAAC
AGTGTAGGCTTATTGTATTTTCATGTAGAGTAGGACCCAAAAATCCACCCAAAGTCCTTTAT
CTATGCCACATCCTTCTTATCTATACTTCCAGGACACTTTTTCTTCCTTATGATAAGGCTCT
CTCTCTCTCCACACACACACACACACACACACACACACACACACACACACACACAAACACAC
ACCCCGCCAACCAAGGTGCATGTAAAAAGATGTAGATTCCTCTGCCTTTCTCATCTACACAG
CCCAGGAGGGTAAGTTAATATAAGAGGGATTTATTGGTAAGAGATGATGCTTAATCTGTTTA
ACACTGGGCCTCAAAGAGAGAATTTCTTTTCTTCTGTACTTATTAAGCACCTATTATGTGTT
GAGCTTATATATACAAAGGGTTATTATATGCTAATATAGTAATAGTAATGGTGGTTGGTACT
ATGGTAATTACCATAAAAATTATTATCCTTTTAAAATAAAGCTAATTATTATTGGATCTTTT
TTAGTATTCATTTTATGTTTTTTATGTTTTTGATTTTTTAAAAGACAATCTCACCCTGTTAC
CCAGGCTGGAGTGCAGTGGTGCAATCATAGCTTTCTGCAGTCTTGAACTCCTGGGCTCAAGC
AATCCTCCTGCCTTGGCCTCCCAAAGTGTTGGGATACAGTCATGAGCCACTGCATCTGGCCT
AGGATCCATTTAGATTAAAATATGCATTTTAAATTTTAAAATAATATGGCTAATTTTTACCT
TATGTAATGTGTATACTGGCAATAAATCTAGTTTGCTGCCTAAAGTTTAAAGTGCTTTCCAG
TAAGCTTCATGTACGTGAGGGGAGACATTTAAAGTGAAACAGACAGCCAGGTGTGGTGGCTC
ACGCCTGTAATCCCAGCACTCTGGGAGGCTGAGGTGGGTGGATCGCTTGAGCCCTGGAGTTC
AAGACCAGCCTGAGCAACATGGCAAAACGCTGTTTCTATAACAAAAATTAGCCGGGCATGGT
GGCATGTGCCTGTGGTCCCAGCTACTAGGGGGCTGAGGCAGGAGAATCGTTGGAGCCCAGGA
GGTCAAGGCTGCACTGAGCAGTGCTTGCGCCACTGCACTCCAGCCTGGGTGACAGGACCAGA
CCTTGCCTCAAAAAAATAAGAAGAAAAATTAAAAATAAATGGAAACAACTACAAAGAGCTGT
TGTCCTAGATGAGCTACTTAGTTAGGCTGATATTTTGGTATTTAACTTTTAAAGTCAGGGTC
TGTCACCTGCACTACATTATTAAAATATCAATTCTCAATGTATATCCACACAAAGACTGGTA
CGTGAATGTTCATAGTACCTTTATTCACAAAACCCCAAAGTAGAGACTATCCAAATATCCAT
CAACAAGTGAACAAATAAACAAAATGTGCTATATCCATGCAATGGAATACCACCCTGCAGTA
CAAAGAAGCTACTTGGGGATGAATCCCAAAGTCATGACGCTAAATGAAAGAGTCAGACATGA
AGGAGGAGATAATGTATGCCATACGAAATTCTAGAAAATGAAAGTAACTTATAGTTACAGAA
AGCAAATCAGGGCAGGCATAGAGGCTCACACCTGTAATCCCAGCACTTTGAGAGGCCACGTG
GGAAGATTGCTAGAACTCAGGAGTTCAAGACCAGCCTGGGCAACACAGTGAAACTCCATTCT
CCACAAAAATGGGAAAAAAAGAAAGCAAATCAGTGGTTGTCCTGTGGGGAGGGGAAGGACTG
CAAAGAGGGAAGAAGCTCTGGTGGGGTGAGGGTGGTGATTCAGGTTCTGTATCCTGACTGTG
GTAGCAGTTTGGGGTGTTTACATCCAAAAATATTCGTAGAATTATGCATCTTAAATGGGTGG
AGTTTACTGTATGTAAATTATACCTCAATGTAAGAAAAAATAATGTGTAAGAAAACTTTCAA
TTCTCTTGCCAGCAAACGTTATTCAAATTCCTGAGCCCTTTACTTCGCAAATTCTCTGCACT
TCTGCCCCGTACCATTAGGTGACAGCACTAGCTCCACAAATTGGATAAATGCATTTCTGGAA
AAGACTAGGGACAAAATCCAGGCATCACTTGTGCTTTCATATCAACCATGCTGTACAGCTTG
TGTTGCTGTCTGCAGCTGCAATGGGGACTCTTGATTTCTTTAAGGAAACTTGGGTTACCAGA
GTATTTCCACAAATGCTATTCAAATTAGTGCTTATGATATGCAAGACACTGTGCTAGGAGCC
AGAAAACAAAGAGGAGGAGAAATCAGTCATTATGTGGGAACAACATAGCAAGATATTTAGAT
CATTTTGACTAGTTAAAAAAGCAGCAGAGTACAAAATCACACATGCAATCAGTATAATCCAA
ATCATGTAAATATGTGCCTGTAGAAAGACTAGAGGAATAAACACAAGAATCTTAACAGTCAT
TGTCATTAGACACTAAGTCTAATTATTATTATTAGACACTATGATATTTGAGATTTAAAAAA
TCTTTAATATTTTAAAATTTAGAGCTCTTCTATTTTTCCATAGTATTCAAGTTTGACAATGA
TCAAGTATTACTCTTTCTTTTTTTTTTTTTTTTTTTTTTTTTGAGATGGAGTTTTGGTCTTG
TTGCCCATGCTGGAGTGGAATGGCATGACCATAGCTCACTGCAACCTCCACCTCCTGGGTTC
AAGCAAAGCTGTCGCCTCAGCCTCCCGGGTAGATGGGATTACAGGCGCCCACCACCACACTC
GGCTAATGTTTGTATTTTTAGTAGAGATGGGGTTTCACCATGTTGGCCAGGCTGGTCTCAAA
CTCCTGACCTCAGAGGATCCACCTGCCTCAGCCTCCCAAAGTGCTGGGATTACAGATGTAGG
CCACTGCGCCCGGCCAAGTATTGCTCTTATACATTAAAAAACAGGTGTGAGCCACTGCGCCC
AGCCAGGTATTGCTCTTATACATTAAAAAATAGGCCGGTGCAGTGGCTCACGCCTGTAATCC
CAGCACTTTGGGAAGCCAAGGCGGGCAGAACACCCGAGGTCAGGAGTCCAAGGCCAGCCTGG
CCAAGATGGTGAAACCCCGTCTCTATTAAAAATACAAACATTACCTGGGCATGATGGTGGGC
GCCTGTAATCCCAGCTACTCAGGAGGCTGAGGCAGGAGGATCCGCGGAGCCTGGCAGATCTG
CCTGAGCCTGGGAGGTTGAGGCTACAGTAAGCCAAGATCATGCCAGTATACTTCAGCCTGGG
CGACAAAGTGAGACCGTAACAAAAAAAAAAAAATTTAAAAAAAGAAATTTAGATCAAGATCC
AACTGTAAAAAGTGGCCTAAACACCACATTAAAGAGTTTGGAGTTTATTCTGCAGGCAGAAG
AGAACCATCAGGGGGTCTTCAGCATGGGAATGGCATGGTGCACCTGGTTTTTGTGAGATCAT
GGTGGTGACAGTGTGGGGAATGTTATTTTGGAGGGACTGGAGGCAGACAGACCGGTTAAAAG
GCCAGCACAACAGATAAGGAGGAAGAAGATGAGGGCTTGGACCGAAGCAGAGAAGAGCAAAC
AGGGAAGGTACAAATTCAAGAAATATTGGGGGGTTTGAATCAACACATTTAGATGATTAATT
AAATATGAGGACTGAGGAATAAGAAATGAGTCAAGGATGGTTCCAGGCTGCTAGGCTGCTTA
CCTGAGGTGGCAAAGTCGGGAGGAGTGGCAGTTTAGGACAGGGGGCAGTTGAGGAATATTGT
TTTGATCATTTTGAGTTTGAGGTACAAGTTGGACACTTAGGTAAAGACTGGAGGGGAAATCT
GAATATACAATTATGGGACTGAGGAACAAGTTTATTTTATTTTTTGTTTCGTTTTCTTGTTG
AAGAACAAATTTAATTGTAATCCCAAGTCATCAGCATCTAGAAGACAGTGGCAGGAGGTGAC
TGTCTTGTGGGTAAGGGTTTGGGGTCCTTGATGAGTATCTCTCAATTGGCCTTAAATATAAG
CAGGAAAAGGAGTTTATGATGGATTCCAGGCTCAGCAGGGCTCAGGAGGGCTCAGGCAGCCA
GCAGAGGAAGTCAGAGCATCTTCTTTGGTTTAGCCCAAGTAATGACTTCCTTAAAAAGCTGA
AGGAAAATCCAGAGTGACCAGATTATAAACTGTACTCTTGCATTTTCTCTCCCTCCTCTCAC
CCACAGCCTCTTGATGAACCGGAGGAAGTTTCTTTACCAATTCAAAAATGTCCGCTGGGCTA
AGGGTCGGCGTGAGACCTACCTGTGCTACGTAGTGAAGAGGCGTGACAGTGCTACATCCTTT
TCACTGGACTTTGGTTATCTTCGCAATAAGGTATCAATTAAAGTCGGCTTTGCAAGCAGTTT
AATGGTCAACTGTGAGTGCTTTTAGAGCCACCTGCTGATGGTATTACTTCCATCCTTTTTTG
GCATTTGTGTCTCTATCACATTCCTCAAATCCTTTTTTTTATTTCTTTTTCCATGTCCATGC
ACCCATATTAGACATGGCCCAAAATATGTGATTTAATTCCTCCCCAGTAATGCTGGGCACCC
TAATACCACTCCTTCCTTCAGTGCCAAGAACAACTGCTCCCAAACTGTTTACCAGCTTTCCT
CAGCATCTGAATTGCCTTTGAGATTAATTAAGCTAAAAGCATTTTTATATGGGAGAATATTA
TCAGCTTGTCCAAGCAAAAATTTTAAATGTGAAAAACAAATTGTGTCTTAAGCATTTTTGAA
AATTAAGGAAGAAGAATTTGGGAAAAAATTAACGGTGGCTCAATTCTGTCTTCCAAATGATT
TCTTTTCCCTCCTACTCACATGGGTCGTAGGCCAGTGAATACATTCAACATGGTGATCCCCA
GAAAACTCAGAGAAGCCTCGGCTGATGATTAATTAAATTGATCTTTCGGCTACCCGAGAGAA
TTACATTTCCAAGAGACTTCTTCACCAAAATCCAGATGGGTTTACATAAACTTCTGCCCACG
GGTATCTCCTCTCTCCTAACACGCTGTGACGTCTGGGCTTGGTGGAATCTCAGGGAAGCATC
CGTGGGGTGGAAGGTCATCGTCTGGCTCGTTGTTTGATGGTTATATTACCATGCAATTTTCT
TTGCCTACATTTGTATTGAATACATCCCAATCTCCTTCCTATTCGGTGACATGACACATTCT
ATTTCAGAAGGCTTTGATTTTATCAAGCACTTTCATTTACTTCTCATGGCAGTGCCTATTAC
TTCTCTTACAATACCCATCTGTCTGCTTTACCAAAATCTATTTCCCCTTTTCAGATCCTCCC
AAATGGTCCTCATAAACTGTCCTGCCTCCACCTAGTGGTCCAGGTATATTTCCACAATGTTA
CATCAACAGGCACTTCTAGCCATTTTCCTTCTCAAAAGGTGCAAAAAGCAACTTCATAAACA
CAAATTAAATCTTCGGTGAGGTAGTGTGATGCTGCTTCCTCCCAACTCAGCGCACTTCGTCT
TCCTCATTCCACAAAAACCCATAGCCTTCCTTCACTCTGCAGGACTAGTGCTGCCAAGGGTT
CAGCTCTACCTACTGGTGTGCTCTTTTGAGCAAGTTGCTTAGCCTCTCTGTAACACAAGGAC
AATAGCTGCAAGCATCCCCAAAGATCATTGCAGGAGACAATGACTAAGGCTACCAGAGCCGC
AATAAAAGTCAGTGAATTTTAGCGTGGTCCTCTCTGTCTCTCCAGAACGGCTGCCACGTGGA
ATTGCTCTTCCTCCGCTACATCTCGGACTGGGACCTAGACCCTGGCCGCTGCTACCGCGTCA
CCTGGTTCACCTCCTGGAGCCCCTGCTACGACTGTGCCCGACATGTGGCCGACTTTCTGCGA
GGGAACCCCAACCTCAGTCTGAGGATCTTCACCGCGCGCCTCTACTTCTGTGAGGACCGCAA
GGCTGAGCCCGAGGGGCTGCGGCGGCTGCACCGCGCCGGGGTGCAAATAGCCATCATGACCT
TCAAAGGTGCGAAAGGGCCTTCCGCGCAGGCGCAGTGCAGCAGCCCGCATTCGGGATTGCGA
TGCGGAATGAATGAGTTAGTGGGGAAGCTCGAGGGGAAGAAGTGGGCGGGGATTCTGGTTCA
CCTCTGGAGCCGAAATTAAAGATTAGAAGCAGAGAAAAGAGTGAATGGCTCAGAGACAAGGC
CCCGAGGAAATGAGAAAATGGGGCCAGGGTTGCTTCTTTCCCCTCGATTTGGAACCTGAACT
GTCTTCTACCCCCATATCCCCGCCTTTTTTTCCTTTTTTTTTTTTTGAAGATTATTTTTACT
GCTGGAATACTTTTGTAGAAAACCACGAAAGAACTTTCAAAGCCTGGGAAGGGCTGCATGAA
AATTCAGTTCGTCTCTCCAGACAGCTTCGGCGCATCCTTTTGGTAAGGGGCTTCCTCGCTTT
TTAAATTTTCTTTCTTTCTCTACAGTCTTTTTTGGAGTTTCGTATATTTCTTATATTTTCTT
ATTGTTCAATCACTCTCAGTTTTCATCTGATGAAAACTTTATTTCTCCTCCACATCAGCTTT
TTCTTCTGCTGTTTCACCATTCAGAGCCCTCTGCTAAGGTTCCTTTTCCCTCCCTTTTCTTT
CTTTTGTTGTTTCACATCTTTAAATTTCTGTCTCTCCCCAGGGTTGCGTTTCCTTCCTGGTC
AGAATTCTTTTCTCCTTTTTTTTTTTTTTTTTTTTTTTTTTTAAACAAACAAACAAAAAACC
CAAAAAAACTCTTTCCCAATTTACTTTCTTCCAACATGTTACAAAGCCATCCACTCAGTTTA
GAAGACTCTCCGGCCCCACCGACCCCCAACCTCGTTTTGAAGCCATTCACTCAATTTGCTTC
TCTCTTTCTCTACAGCCCCTGTATGAGGTTGATGACTTACGAGACGCATTTCGTACTTTGGG
ACTTTGATAGCAACTTCCAGGAATGTCACACACGATGAAATATCTCTGCTGAAGACAGTGGA
TAAAAAACAGTCCTTCAAGTCTTCTCTGTTTTTATTCTTCAACTCTCACTTTCTTAGAGTTT
ACAGAAAAAATATTTATATACGACTCTTTAAAAAGATCTATGTCTTGAAAATAGAGAAGGAA
CACAGGTCTGGCCAGGGACGTGCTGCAATTGGTGCAGTTTTGAATGCAACATTGTCCCCTAC
TGGGAATAACAGAACTGCAGGACCTGGGAGCATCCTAAAGTGTCAACGTTTTTCTATGACTT
TTAGGTAGGATGAGAGCAGAAGGTAGATCCTAAAAAGCATGGTGAGAGGATCAAATGTTTTT
ATATCAACATCCTTTATTATTTGATTCATTTGAGTTAACAGTGGTGTTAGTGATAGATTTTT
CTATTCTTTTCCCTTGACGTTTACTTTCAAGTAACACAAACTCTTCCATCAGGCCATGATCT
ATAGGACCTCCTAATGAGAGTATCTGGGTGATTGTGACCCCAAACCATCTCTCCAAAGCATT
AATATCCAATCATGCGCTGTATGTTTTAATCAGCAGAAGCATGTTTTTATGTTTGTACAAAA
GAAGATTGTTATGGGTGGGGATGGAGGTATAGACCATGCATGGTCACCTTCAAGCTACTTTA
ATAAAGGATCTTAAAATGGGCAGGAGGACTGTGAACAAGACACCCTAATAATGGGTTGATGT
CTGAAGTAGCAAATCTTCTGGAAACGCAAACTCTTTTAAGGAAGTCCCTAATTTAGAAACAC
CCACAAACTTCACATATCATAATTAGCAAACAATTGGAAGGAAGTTGCTTGAATGTTGGGGA
GAGGAAAATCTATTGGCTCTCGTGGGTCTCTTCATCTCAGAAATGCCAATCAGGTCAAGGTT
TGCTACATTTTGTATGTGTGTGATGCTTCTCCCAAAGGTATATTAACTATATAAGAGAGTTG
TGACAAAACAGAATGATAAAGCTGCGAACCGTGGCACACGCTCATAGTTCTAGCTGCTTGGG
AGGTTGAGGAGGGAGGATGGCTTGAACACAGGTGTTCAAGGCCAGCCTGGGCAACATAACAA
GATCCTGTCTCTCAAAAAAAAAAAAAAAAAAAAGAAAGAGAGAGGGCCGGGCGTGGTGGCTC
ACGCCTGTAATCCCAGCACTTTGGGAGGCCGAGCCGGGCGGATCACCTGTGGTCAGGAGTTT
GAGACCAGCCTGGCCAACATGGCAAAACCCCGTCTGTACTCAAAATGCAAAAATTAGCCAGG
CGTGGTAGCAGGCACCTGTAATCCCAGCTACTTGGGAGGCTGAGGCAGGAGAATCGCTTGAA
CCCAGGAGGTGGAGGTTGCAGTAAGCTGAGATCGTGCCGTTGCACTCCAGCCTGGGCGACAA
GAGCAAGACTCTGTCTCAGAAAAAAAAAAAAAAAAGAGAGAGAGAGAGAAAGAGAACAATAT
TTGGGAGAGAAGGATGGGGAAGCATTGCAAGGAAATTGTGCTTTATCCAACAAAATGTAAGG
AGCCAATAAGGGATCCCTATTTGTCTCTTTTGGTGTCTATTTGTCCCTAACAACTGTCTTTG
ACAGTGAGAAAAATATTCAGAATAACCATATCCCTGTGCCGTTATTACCTAGCAACCCTTGC
AATGAAGATGAGCAGATCCACAGGAAAACTTGAATGCACAACTGTCTTATTTTAATCTTATT
GTACATAAGTTTGTAAAAGAGTTAAAAATTGTTACTTCATGTATTCATTTATATTTTATATT
ATTTTGCGTCTAATGATTTTTTATTAACATGATTTCCTTTTCTGATATATTGAAATGGAGTC
TCAAAGCTTCATAAATTTATAACTTTAGAAATGATTCTAATAACAACGTATGTAATTGTAAC
ATTGCAGTAATGGTGCTACGAAGCCATTTCTCTTGATTTTTAGTAAACTTTTATGACAGCAA
ATTTGCTTCTGGCTCACTTTCAATCAGTTAAATAAATGATAAATAATTTTGGAAGCTGTGAA
GATAAAATACCAAATAAAATAATATAAAAGTGATTTATATGAAGTTAAAATAAAAAATCAGT
ATGATGGAATAAACTTG

Other exemplary deaminases that can be fused to Cas9 according to aspects of this disclosure are provided below. In embodiments, the deaminases are activation-induced deaminases (AID). It should be understood that, in some embodiments, the active domain of the respective sequence can be used, e.g., the domain without a localizing signal (nuclear localization sequence, without nuclear export signal, cytoplasmic localizing signal).

Human AID:
MDSLLMNRRKFLYQFKNVRWAKGRRETYLCYVVKRRDSATSFSLDFGYLRNKNGCHVELLFL
RYISDWDLDPGRCYRVTWFTSWSPCYDCARHVADFLRGNPNLSLRIFTARLYFCEDRKAEPE
GLRRLHRAGVQIAIMTFKDYFYCWNTFVENHERTFKAWEGLHENSVRLSRQLRRILLPLYEV
DDLRDAFRTLGL
(underline: nuclear localization sequence; double underline: nuclear
export signal)
Mouse AID:
MDSLLMKQKKFLYHFKNVRWAKGRHETYLCYVVKRRDSATSCSLDFGHLRNKSGCHVELLFL
RYISDWDLDPGRCYRVTWFTSWSPCYDCARHVAEFLRWNPNLSLRIFTARLYFCEDRKAEPE
GLRRLHRAGVQIGIMTFKDYFYCWNTFVENRERTFKAWEGLHENSVRLTRQLRRILLPLYEV
DDLRDAFRMLGF
(underline: nuclear localization sequence; double underline: nuclear
export signal)
Canine AID:
MDSLLMKQRKFLYHFKNVRWAKGRHETYLCYVVKRRDSATSFSLDFGHLRNKSGCHVELLFL
RYISDWDLDPGRCYRVTWFTSWSPCYDCARHVADFLRGYPNLSLRIFAARLYFCEDRKAEPE
GLRRLHRAGVQIAIMTFKDYFYCWNTFVENREKTFKAWEGLHENSVRLSRQLRRILLPLYEV
DDLRDAFRTLGL
(underline: nuclear localization sequence; double underline: nuclear
export signal)
Bovine AID:
MDSLLKKQRQFLYQFKNVRWAKGRHETYLCYVVKRRDSPTSFSLDFGHLRNKAGCHVELLFL
RYISDWDLDPGRCYRVTWFTSWSPCYDCARHVADFLRGYPNLSLRIFTARLYFCDKERKAEP
EGLRRLHRAGVQIAIMTFKDYFYCWNTFVENHERTFKAWEGLHENSVRLSRQLRRILLPLYE
VDDLRDAFRTLGL
(underline: nuclear localization sequence; double underline: nuclear
export signal)
Rat AID:
MAVGSKPKAALVGPHWERERIWCFLCSTGLGTQQTGQTSRWLRPAATQDPVSPPRSLLMKQR
KFLYHFKNVRWAKGRHETYLCYVVKRRDSATSFSLDFGYLRNKSGCHVELLFLRYISDWDLD
PGRCYRVTWFTSWSPCYDCARHVADFLRGNPNLSLRIFTARLTGWGALPAGLMSPARPSDYF
YCWNTFVENHERTFKAWEGLHENSVRLSRRLRRILLPLYEVDDLRDAFRTLGL
(underline: nuclear localization sequence; double underline:
nuclear export signal)
clAID (Canis lupus familiaris):
MDSLLMKQRKFLYHFKNVRWAKGRHETYLCYVVKRRDSATSFSLDFGHLRNKSGCHVELLFLRYISDW
DLDPGRCYRVTWFTSWSPCYDCARHVADFLRGYPNLSLRIFAARLYFCEDRKAEPEGLRRLHRAGVQI
AIMTFKDYFYCWNTFVENREKTFKAWEGLHENSVRLSRQLRRILLPLYEVDDLRDAFRTLGL
btAID (Bos Taurus):
MDSLLKKQRQFLYQFKNVRWAKGRHETYLCYVVKRRDSPTSFSLDFGHLRNKAGCHVELLFLRYISDW
DLDPGRCYRVTWFTSWSPCYDCARHVADFLRGYPNLSLRIFTARLYFCDKERKAEPEGLRRLHRAGVQ
IAIMTFKDYFYCWNTFVENHERTFKAWEGLHENSVRLSRQLRRILLPLYEVDDLRDAFRTLGL
mAID (Mus musculus):
MDSLLMNRRKFLYQFKNVRWAKGRRETYLCYVVKRRDSATSFSLDFGYLRNKNGCHVELLFLRYISDW
DLDPGRCYRVTWFTSWSPCYDCARHVADFLRGNPNLSLRIFTARLYFCEDRKAEPEGLRRLHRAGVQI
AIMTFKDYFYCWNTFVENHERTFKAWEGLHENSVRLSRQLRRILLPLYEVDDLRDAFRTLGL
rAPOBEC-1 (Rattus norvegicus):
MSSETGPVAVDPTLRRRIEPHEFEVFFDPRELRKETCLLYEINWGGRHSIWRHTSQNTNKHVEVNFIE
KFTTERYFCPNTRCSITWFLSWSPCGECSRAITEFLSRYPHVTLFIYIARLYHHADPRNRQGLRDLIS
SGVTIQIMTEQESGYCWRNFVNYSPSNEAHWPRYPHLWVRLYVLELYCIILGLPPCLNILRRKQPQLT
FFTIALQSCHYQRLPPHILWATGLK
maAPOBEC-1 (Mesocricetus auratus):
MSSETGPVVVDPTLRRRIEPHEFDAFFDQGELRKETCLLYEIRWGGRHNIWRHTGQNTSRHVEINFIE
KFTSERYFYPSTRCSIVWFLSWSPCGECSKAITEFLSGHPNVTLFIYAARLYHHTDQRNRQGLRDLIS
RGVTIRIMTEQEYCYCWRNFVNYPPSNEVYWPRYPNLWMRLYALELYCIHLGLPPCLKIKRRHQYPLT
FFRLNLQSCHYQRIPPHILWATGFI
ppAPOBEC-1 (Pongo pygmaeus):
MTSEKGPSTGDPTLRRRIESWEFDVFYDPRELRKETCLLYEIKWGMSRKIWRSSGKNTINHVEVNFIK
KFTSERRFHSSISCSITWFLSWSPCWECSQAIREFLSQHPGVTLVIYVARLFWHMDQRNRQGLRDLVN
SGVTIQIMRASEYYHCWRNFVNYPPGDEAHWPQYPPLWMMLYALELHCIILSLPPCLKISRRWQNHLA
FFRLHLQNCHYQTIPPHILLATGLIHPSVTWR
ocAPOBEC1 (Oryctolagus cuniculus):
MASEKGPSNKDYTLRRRIEPWEFEVFFDPQELRKEACLLYEIKWGASSKTWRSSGKNTINHVEVNFLE
KLTSEGRLGPSTCCSITWFLSWSPCWECSMAIREFLSQHPGVTLIIFVARLFQHMDRRNRQGLKDLVT
SGVTVRVMSVSEYCYCWENFVNYPPGKAAQWPRYPPRWMLMYALELYCIILGLPPCLKISRRHQKQLT
FFSLTPQYCHYKMIPPYILLATGLLQPSVPWR
mdAPOBEC-1 (Monodelphis domestica):
MNSKTGPSVGDATLRRRIKPWEFVAFFNPQELRKETCLLYEIKWGNQNIWRHSNQNTSQHAEINFMEK
FTAERHFNSSVRCSITWFLSWSPCWECSKAIRKFLDHYPNVTLAIFISRLYWHMDQQHRQGLKELVHS
GVTIQIMSYSEYHYCWRNFVDYPQGEEDYWPKYPYLWIMLYVLELHCIILGLPPCLKISGSHSNQLAL
FSLDLQDCHYQKIPYNVLVATGLVQPFVTWR
ppAPOBEC-2 (Pongo pygmaeus):
MAQKEEAAAATEAASQNGEDLENLDDPEKLKELIELPPFEIVTGERLPANFFKFQFRNVEYSSGRNKT
FLCYVVEAQGKGGQVQASRGYLEDEHAAAHAEEAFFNTILPAFDPALRYNVTWYVSSSPCAACADRII
KTLSKTKNLRLLILVGRLFMWEELEIQDALKKLKEAGCKLRIMKPQDFEYVWQNFVEQEEGESKAFQP
WEDIQENFLYYEEKLADILK
btAPOBEC-2 (Bos Taurus):
MAQKEEAAAAAEPASQNGEEVENLEDPEKLKELIELPPFEIVTGERLPAHYFKFQFRNVEYSSGRNKT
FLCYVVEAQSKGGQVQASRGYLEDEHATNHAEEAFFNSIMPTFDPALRYMVTWYVSSSPCAACADRIV
KTLNKTKNLRLLILVGRLFMWEEPEIQAALRKLKEAGCRLRIMKPQDFEYIWQNFVEQEEGESKAFEP
WEDIQENFLYYEEKLADILK
mAPOBEC-3-(1) (Mus musculus):
MQPQRLGPRAGMGPFCLGCSHRKCYSPIRNLISQETFKFHFKNLGYAKGRKDTFLCYEVTRKDCDSPV
SLHHGVFKNKDNIHAEICFLYWFHDKVLKVLSPREEFKITWYMSWSPCFECAEQIVRFLATHHNLSLD
IFSSRLYNVQDPETQQNLCRLVQEGAQVAAMDLYEFKKCWKKFVDNGGRRFRPWKRLLTNFRYQDSKL
QEILRPCYISVPSSSSSTLSNICLTKGLPETRFWVEGRRMDPLSEEEFYSQFYNQRVKHLCYYHRMKP
YLCYQLEQFNGQAPLKGCLLSEKGKQHAEILFLDKIRSMELSQVTITCYLTWSPCPNCAWQLAAFKRD
RPDLILHIYTSRLYFHWKRPFQKGLCSLWQSGILVDVMDLPQFTDCWTNFVNPKRPFWPWKGLEIISR
RTQRRLRRIKESWGLQDLVNDFGNLQLGPPMS
Mouse APOBEC-3-(2):
MGPFCLGCSHRKCYSPIRNLISQETFKFHFKNLGYAKGRKDTFLCYEVTRKDCDSPVSLHHGVFKNKD
NIHAEICFLYWFHDKVLKVLSPREEFKITWYMSWSPCFECAEQIVRFLATHHNLSLDIFSSRLYNVQD
PETQQNLCRLVQEGAQVAAMDLYEFKKCWKKFVDNGGRRFRPWKRLLTNFRYQDSKLQEILRPCYIPV
PSSSSSTLSNICLTKGLPETRFCVEGRRMDPLSEEEFYSQFYNQRVKHLCYYHRMKPYLCYQLEQFNG
QAPLKGCLLSEKGKQHAEILFLDKIRSMELSQVTITCYLTWSPCPNCAWQLAAFKRDRPDLILHIYTS
RLYFHWKRPFQKGLCSLWQSGILVDVMDLPQFTDCWINFVNPKRPFWPWKGLEIISRRTQRRLRRIKE
SWGLQDLVNDFGNLQLGPPMS
(italic: nucleic acid editing domain)
Rat APOBEC-3:
MGPFCLGCSHRKCYSPIRNLISQETFKFHFKNRLRYAIDRKDTFLCYEVTRKDCDSPVSLHHGVFKNK
DNIHAEICFLYWFHDKVLKVLSPREEFKITWYMSWSPCFECAEQVLRFLATHHNLSLDIFSSRLYNIR
DPENQQNLCRLVQEGAQVAAMDLYEFKKCWKKFVDNGGRRFRPWKKLLTNFRYQDSKLQEILRPCYIP
VPSSSSSTLSNICLTKGLPETRFCVERRRVHLLSEEEFYSQFYNQRVKHLCYYHGVKPYLCYQLEQFN
GQAPLKGCLLSEKGKQHAEILFLDKIRSMELSQVIITCYLTWSPCPNCAWQLAAFKRDRPDLILHIYT
SRLYFHWKRPFQKGLCSLWQSGILVDVMDLPQFTDCWTNFVNPKRPFWPWKGLEIISRRTQRRLHRIK
ESWGLQDLVNDFGNLQLGPPMS
(italic: nucleic acid editing domain)
hAPOBEC-3A (Homo sapiens):
MEASPASGPRHLMDPHIFTSNFNNGIGRHKTYLCYEVERLDNGTSVKMDQHRGFLHNQAKNLLCGFYG
RHAELRFLDLVPSLQLDPAQIYRVTWFISWSPCFSWGCAGEVRAFLQENTHVRLRIFAARIYDYDPLY
KEALQMLRDAGAQVSIMTYDEFKHCWDTFVDHQGCPFQPWDGLDEHSQALSGRLRAILQNQGN
hAPOBEC-3F (Homo sapiens):
MKPHFRNTVERMYRDTFSYNFYNRPILSRRNTVWLCYEVKTKGPSRPRLDAKIFRGQVYSQPEHHAEM
CFLSWFCGNQLPAYKCFQITWFVSWTPCPDCVAKLAEFLAEHPNVTLTISAARLYYYWERDYRRALCR
LSQAGARVKIMDDEEFAYCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFLSWFCDDILSPNTNYEVT
WYTSWSPCPECAGEVAEFLARHSNVNLTIFTARLYYFWDTDYQEGLRSLSQEGASVEIMGYKDFKYCW
ENFVYNDDEPFKPWKGLKYNFLFLDSKLQEILE
Rhesus macaque APOBEC-3G:
MVEPMDPRTFVSNFNNRPILSGLNTVWLCCEVKTKDPSGPPLDAKIFQGKVYSKAKYHPEMRFLRWFH
KWRQLHHDQEYKVTWYVSWSPCTRCANSVATFLAKDPKVTLTIFVARLYYFWKPDYQQALRILCQKRG
GPHATMKIMNYNEFQDCWNKFVDGRGKPFKPRNNLPKHYTLLQATLGELLRHLMDPGTFTSNFNNKPW
VSGQHETYLCYKVERLHNDTWVPLNQHRGFLRNQAPNIHGFPKGRHAELCFLDLIPFWKLDGQQYRVT
CFTSWSPCFSCAQEMAKFISNNEHVSLCIFAARIYDDQGRYQEGLRALHRDGAKIAMMNYSEFEYCWD
TFVDRQGRPFQPWDGLDEHSQALSGRLRAI
(italic: nucleic acid editing domain; underline: cytoplasmic
localization signal)
Chimpanzee APOBEC-3G:
MKPHFRNPVERMYQDTFSDNFYNRPILSHRNTVWLCYEVKTKGPSRPPLDAKIFRGQVYSKLKYHPEM
RFFHWFSKWRKLHRDQEYEVTWYISWSPCTKCTRDVATFLAEDPKVTLTIFVARLYYFWDPDYQEALR
SLCQKRDGPRATMKIMNYDEFQHCWSKFVYSQRELFEPWNNLPKYYILLHIMLGEILRHSMDPPTFTS
NFNNELWVRGRHETYLCYEVERLHNDTWVLLNQRRGFLCNQAPHKHGFLEGRHAELCFLDVIPFWKLD
LHQDYRVTCFTSWSPCFSCAQEMAKFISNNKHVSLCIFAARIYDDQGRCQEGLRTLAKAGAKISIMTY
SEFKHCWDTFVDHQGCPFQPWDGLEEHSQALSGRLRAILQNQGN
(italic: nucleic acid editing domain; underline: cytoplasmic
localization signal)
Green monkey APOBEC-3G:
MNPQIRNMVEQMEPDIFVYYFNNRPILSGRNTVWLCYEVKTKDPSGPPLDANIFQGKLYPEAKDHPEM
KFLHWFRKWRQLHRDQEYEVTWYVSWSPCTRCANSVATFLAEDPKVTLTIFVARLYYFWKPDYQQALR
ILCQERGGPHATMKIMNYNEFQHCWNEFVDGQGKPFKPRKNLPKHYTLLHATLGELLRHVMDPGTFTS
NFNNKPWVSGQRETYLCYKVERSHNDTWVLLNQHRGFLRNQAPDRHGFPKGRHAELCFLDLIPFWKLD
DQQYRVTCFTSWSPCFSCAQKMAKFISNNKHVSLCIFAARIYDDQGRCQEGLRTLHRDGAKIAVMNYS
EFEYCWDTFVDRQGRPFQPWDGLDEHSQALSGRLRAI
(italic: nucleic acid editing domain; underline: cytoplasmic
localization signal)
Human APOBEC-3G:
MKPHFRNTVERMYRDTFSYNFYNRPILSRRNTVWLCYEVKTKGPSRPPLDAKIFRGQVYSELKYHPEM
RFFHWFSKWRKLHRDQEYEVTWYISWSPCTKCTRDMATFLAEDPKVTLTIFVARLYYFWDPDYQEALR
SLCQKRDGPRATMKIMNYDEFQHCWSKFVYSQRELFEPWNNLPKYYILLHIMLGEILRHSMDPPTFTF
NFNNEPWVRGRHETYLCYEVERMHNDTWVLLNQRRGFLCNQAPHKHGFLEGRHAELCFLDVIPFWKLD
LDQDYRVTCFTSWSPCFSCAQEMAKFISKNKHVSLCIFTARIYDDQGRCQEGLRTLAEAGAKISIMTY
SEFKHCWDTFVDHQGCPFQPWDGLDEHSQDLSGRLRAILQNQEN
(italic: nucleic acid editing domain; underline: cytoplasmic
localization signal)
Human APOBEC-3F:
MKPHFRNTVERMYRDTFSYNFYNRPILSRRNTVWLCYEVKTKGPSRPRLDAKIFRGQVYSQPEHHAEM
CFLSWFCGNQLPAYKCFQITWFVSWTPCPDCVAKLAEFLAEHPNVTLTISAARLYYYWERDYRRALCR
LSQAGARVKIMDDEEFAYCWENFVYSEGQPFMPWYKFDDNYAFLHRTLKEILRNPMEAMYPHIFYFHF
KNLRKAYGRNESWLCFTMEVVKHHSPVSWKRGVFRNQVDPETHCHAERCFLSWFCDDILSPNTNYEVT
WYTSWSPCPECAGEVAEFLARHSNVNLTIFTARLYYFWDTDYQEGLRSLSQEGASVEIMGYKDFKYCW
ENFVYNDDEPFKPWKGLKYNFLFLDSKLQEILE
(italic: nucleic acid editing domain)
Human APOBEC-3B:
MNPQIRNPMERMYRDTFYDNFENEPILYGRSYTWLCYEVKIKRGRSNLLWDTGVFRGQVYFKPQYHAE
MCFLSWFCGNQLPAYKCFQITWFVSWTPCPDCVAKLAEFLSEHPNVTLTISAARLYYYWERDYRRALC
RLSQAGARVTIMDYEEFAYCWENFVYNEGQQFMPWYKFDENYAFLHRTLKEILRYLMDPDTFTFNFNN
DPLVLRRRQTYLCYEVERLDNGTWVLMDQHMGFLCNEAKNLLCGFYGRHAELRFLDLVPSLQLDPAQI
YRVTWFISWSPCFSWGCAGEVRAFLQENTHVRLRIFAARIYDYDPLYKEALQMLRDAGAQVSIMTYDE
FEYCWDTFVYRQGCPFQPWDGLEEHSQALSGRLRAILQNQGN
(italic: nucleic acid editing domain)
Rat APOBEC-3B:
MQPQGLGPNAGMGPVCLGCSHRRPYSPIRNPLKKLYQQTFYFHFKNVRYAWGRKNNFLCYEVNGMDCA
LPVPLRQGVFRKQGHIHAELCFIYWFHDKVLRVLSPMEEFKVTWYMSWSPCSKCAEQVARFLAAHRNL
SLAIFSSRLYYYLRNPNYQQKLCRLIQEGVHVAAMDLPEFKKCWNKFVDNDGQPFRPWMRLRINFSFY
DCKLQEIFSRMNLLREDVFYLQFNNSHRVKPVQNRYYRRKSYLCYQLERANGQEPLKGYLLYKKGEQH
VEILFLEKMRSMELSQVRITCYLTWSPCPNCARQLAAFKKDHPDLILRIYTSRLYFWRKKFQKGLCTL
WRSGIHVDVMDLPQFADCWTNFVNPQRPFRPWNELEKNSWRIQRRLRRIKESWGL
Bovine APOBEC-3B:
DGWEVAFRSGTVLKAGVLGVSMTEGWAGSGHPGQGACVWTPGTRNTMNLLREVLFKQQFGNQPRVPAP
YYRRKTYLCYQLKQRNDLTLDRGCFRNKKQRHAERFIDKINSLDLNPSQSYKIICYITWSPCPNCANE
LVNFITRNNHLKLEIFASRLYFHWIKSFKMGLQDLQNAGISVAVMTHTEFEDCWEQFVDNQSRPFQPW
DKLEQYSASIRRRLQRILTAPI
Chimpanzee APOBEC-3B:
MNPQIRNPMEWMYQRTFYYNFENEPILYGRSYTWLCYEVKIRRGHSNLLWDTGVFRGQMYSQPEHHAE
MCFLSWFCGNQLSAYKCFQITWFVSWTPCPDCVAKLAKFLAEHPNVTLTISAARLYYYWERDYRRALC
RLSQAGARVKIMDDEEFAYCWENFVYNEGQPFMPWYKFDDNYAFLHRTLKEIIRHLMDPDTFTFNFNN
DPLVLRRHQTYLCYEVERLDNGTWVLMDQHMGFLCNEAKNLLCGFYGRHAELRFLDLVPSLQLDPAQI
YRVTWFISWSPCFSWGCAGQVRAFLQENTHVRLRIFAARIYDYDPLYKEALQMLRDAGAQVSIMTYDE
FEYCWDTFVYRQGCPFQPWDGLEEHSQALSGRLRAILQVRASSLCMVPHRPPPPPQSPGPCLPLCSEP
PLGSLLPTGRPAPSLPFLLTASFSFPPPASLPPLPSLSLSPGHLPVPSFHSLTSCSIQPPCSSRIRET
EGWASVSKEGRDLG
Human APOBEC-3C:
MNPQIRNPMKAMYPGTFYFQFKNLWEANDRNETWLCFTVEGIKRRSVVSWKTGVFRNQVDSETHCHAE
RCFLSWFCDDILSPNTKYQVTWYTSWSPCPDCAGEVAEFLARHSNVNLTIFTARLYYFQYPCYQEGLR
SLSQEGVAVEIMDYEDFKYCWENFVYNDNEPFKPWKGLKTNFRLLKRRLRESLQ
(italic: nucleic acid editing domain)
Gorilla APOBEC-3C
MNPQIRNPMKAMYPGTFYFQFKNLWEANDRNETWLCFTVEGIKRRSVVSWKTGVFRNQVDSETHCHAE
RCFLSWECDDILSPNTNYQVTWYTSWSPCPECAGEVAEFLARHSNVNLTIFTARLYYFQDTDYQEGLR
SLSQEGVAVKIMDYKDFKYCWENFVYNDDEPFKPWKGLKYNFRFLKRRLQEILE
(italic: nucleic acid editing domain)
Human APOBEC-3A:
MEASPASGPRHLMDPHIFTSNFNNGIGRHKTYLCYEVERLDNGTSVKMDQHRGFLHNQAKNLLCGFYG
RHAELRFLDLVPSLQLDPAQIYRVTWFISWSPCFSWGCAGEVRAFLQENTHVRLRIFAARIYDYDPLY
KEALQMLRDAGAQVSIMTYDEFKHCWDTFVDHQGCPFQPWDGLDEHSQALSGRLRAILQNQGN
(italic: nucleic acid editing domain)
Rhesus macaque APOBEC-3A:
MDGSPASRPRHLMDPNTFTFNFNNDLSVRGRHQTYLCYEVERLDNGTWVPMDERRGFLCNKAKNVPCG
DYGCHVELRFLCEVPSWQLDPAQTYRVTWFISWSPCFRRGCAGQVRVFLQENKHVRLRIFAARIYDYD
PLYQEALRTLRDAGAQVSIMTYEEFKHCWDTFVDRQGRPFQPWDGLDEHSQALSGRLRAILQNQGN
(italic: nucleic acid editing domain)
Bovine APOBEC-3A:
MDEYTFTENFNNQGWPSKTYLCYEMERLDGDATIPLDEYKGFVRNKGLDQPEKPCHAELYFLGKIHSW
NLDRNQHYRLTCFISWSPCYDCAQKLITFLKENHHISLHILASRIYTHNRFGCHQSGLCELQAAGARI
TIMTFEDFKHCWETFVDHKGKPFQPWEGLNVKSQALCTELQAILKTQQN
(italic: nucleic acid editing domain)
Human APOBEC-3H:
MALLTAETFRLQFNNKRRLRRPYYPRKALLCYQLTPQNGSTPTRGYFENKKKCHAEICFINEIKSMGL
DETQCYQVTCYLTWSPCSSCAWELVDFIKAHDHLNLGIFASRLYYHWCKPQQKGLRLLCGSQVPVEVM
GFPKFADCWENFVDHEKPLSFNPYKMLEELDKNSRAIKRRLERIKIPGVRAQGRYMDILCDAEV
(italic: nucleic acid editing domain)
Rhesus macaque APOBEC-3H:
MALLTAKTFSLQFNNKRRVNKPYYPRKALLCYQLTPQNGSTPTRGHLKNKKKDHAEIRFINKIKSMGL
DETQCYQVTCYLTWSPCPSCAGELVDFIKAHRHLNLRIFASRLYYHWRPNYQEGLLLLCGSQVPVEVM
GLPEFTDCWENFVDHKEPPSFNPSEKLEELDKNSQAIKRRLERIKSRSVDVLENGLRSLQLGPVTPSS
SIRNSR
Human APOBEC-3D:
MNPQIRNPMERMYRDTFYDNFENEPILYGRSYTWLCYEVKIKRGRSNLLWDTGVFRGPVLPKRQSNHR
QEVYFRFENHAEMCFLSWFCGNRLPANRRFQITWFVSWNPCLPCVVKVTKFLAEHPNVTLTISAARLY
YYRDRDWRWVLLRLHKAGARVKIMDYEDFAYCWENFVCNEGQPFMPWYKFDDNYASLHRTLKEILRNP
MEAMYPHIFYFHFKNLLKACGRNESWLCFTMEVTKHHSAVFRKRGVFRNQVDPETHCHAERCFLSWFC
DDILSPNTNYEVTWYTSWSPCPECAGEVAEFLARHSNVNLTIFTARLCYFWDTDYQEGLCSLSQEGAS
VKIMGYKDFVSCWKNFVYSDDEPFKPWKGLQINFRLLKRRLREILQ
(italic: nucleic acid editing domain)
Human APOBEC-1:
MTSEKGPSTGDPTLRRRIEPWEFDVFYDPRELRKEACLLYEIKWGMSRKIWRSSGKNTINHVEVNFIK
KFTSERDFHPSMSCSITWFLSWSPCWECSQAIREFLSRHPGVTLVIYVARLFWHMDQQNRQGLRDLVN
SGVTIQIMRASEYYHCWRNFVNYPPGDEAHWPQYPPLWMMLYALELHCIILSLPPCLKISRRWQNHLT
FFRLHLQNCHYQTIPPHILLATGLIHPSVAWR
Mouse APOBEC-1:
MSSETGPVAVDPTLRRRIEPHEFEVFFDPRELRKETCLLYEINWGGRHSVWRHTSQNTSNHVEVNFLE
KFTTERYFRPNTRCSITWFLSWSPCGECSRAITEFLSRHPYVTLFIYIARLYHHTDQRNRQGLRDLIS
SGVTIQIMTEQEYCYCWRNFVNYPPSNEAYWPRYPHLWVKLYVLELYCIILGLPPCLKILRRKQPQLT
FFTITLQTCHYQRIPPHLLWATGLK
Rat APOBEC-1:
MSSETGPVAVDPTLRRRIEPHEFEVFFDPRELRKETCLLYEINWGGRHSIWRHTSQNTNKHVEVNFIE
KFTTERYFCPNTRCSITWFLSWSPCGECSRAITEFLSRYPHVTLFIYIARLYHHADPRNRQGLRDLIS
SGVTIQIMTEQESGYCWRNFVNYSPSNEAHWPRYPHLWVRLYVLELYCIILGLPPCLNILRRKQPQLT
FFTIALQSCHYQRLPPHILWATGLK
Human APOBEC-2:
MAQKEEAAVATEAASQNGEDLENLDDPEKLKELIELPPFEIVTGERLPANFFKFQFRNVEYSSGRNKT
FLCYVVEAQGKGGQVQASRGYLEDEHAAAHAEEAFFNTILPAFDPALRYNVTWYVSSSPCAACADRII
KTLSKTKNLRLLILVGRLFMWEEPEIQAALKKLKEAGCKLRIMKPQDFEYVWQNFVEQEEGESKAFQP
WEDIQENFLYYEEKLADILK
Mouse APOBEC-2:
MAQKEEAAEAAAPASQNGDDLENLEDPEKLKELIDLPPFEIVTGVRLPVNFFKFQFRNVEYSSGRNKT
FLCYVVEVQSKGGQAQATQGYLEDEHAGAHAEEAFFNTILPAFDPALKYNVTWYVSSSPCAACADRIL
KTLSKTKNLRLLILVSRLFMWEEPEVQAALKKLKEAGCKLRIMKPQDFEYIWQNFVEQEEGESKAFEP
WEDIQENFLYYEEKLADILK
Rat APOBEC-2:
MAQKEEAAEAAAPASQNGDDLENLEDPEKLKELIDLPPFEIVTGVRLPVNFFKFQFRNVEYSSGRNKT
FLCYVVEAQSKGGQVQATQGYLEDEHAGAHAEEAFFNTILPAFDPALKYNVTWYVSSSPCAACADRIL
KTLSKTKNLRLLILVSRLFMWEEPEVQAALKKLKEAGCKLRIMKPQDFEYLWQNFVEQEEGESKAFEP
WEDIQENFLYYEEKLADILK
Bovine APOBEC-2:
MAQKEEAAAAAEPASQNGEEVENLEDPEKLKELIELPPFEIVTGERLPAHYFKFQFRNVEYSSGRNKT
FLCYVVEAQSKGGQVQASRGYLEDEHATNHAEEAFFNSIMPTFDPALRYMVTWYVSSSPCAACADRIV
KTLNKTKNLRLLILVGRLFMWEEPEIQAALRKLKEAGCRLRIMKPQDFEYIWQNFVEQEEGESKAFEP
WEDIQENFLYYEEKLADILK
Petromyzon marinus CDA1 (pmCDA1):
MTDAEYVRIHEKLDIYTFKKQFFNNKKSVSHRCYVLFELKRRGERRACFWGYAVNKPQSGTERGIHAE
IFSIRKVEEYLRDNPGQFTINWYSSWSPCADCAEKILEWYNQELRGNGHTLKIWACKLYYEKNARNQI
GLWNLRDNGVGLNVMVSEHYQCCRKIFIQSSHNQ
LNENRWLEKTLKRAEKRRSELSFMIQVKILHTTKSPAV
Human APOBEC3G D316R D317R:
MKPHFRNTVERMYRDTFSYNFYNRPILSRRNTVWLCYEVKTKGPSRPPLDAKIFRGQVYSELKYHPEM
RFFHWFSKWRKLHRDQEYEVTWYISWSPCTKCTRDMATFLAEDPKVTLTIFVARLYYFWDPDYQEALR
SLCQKRDGPRATMKFNYDEFQHCWSKFVYSQRELFEPWNNLPKYYILLHFMLGEILRHSMDPPTFTFN
FNNEPWVRGRHETYLCYEVERMHNDTWVLLNQRRGFLCNQAPHKHGFLEGRHAELCFLDVIPFWKLDL
DQDYRVTCFTSWSPCFSCAQEMAKFISKKHVSLCIFTARIYRRQGRCQEGLRTLAEAGAKISFTYSEF
KHCWDTFVDHQGCPFQPWDGLDEHSQDLSGRLRAILQNQEN
Human APOBEC3G chain A:
MDPPTFTFNFNNEPWWGRHETYLCYEVERMHNDTWVLLNQRRGFLCNQAPHKHGFLEGRHAELCFLDV
IPFWKLDLDQDYRVTCFTSWSPCFSCAQEMAKFISKNKHVSLCIFTARIYDDQGRCQEGLRTLAEAGA
KISFTYSEFKHCWDTFVDHQGCPFQPWDGLDEHSQDLSGRLRAILQ
Human APOBEC3G chain A D120R D121R:
MDPPTFTFNFNNEPWVRGRHETYLCYEVERMHNDTWVLLNQRRGFLCNQAPHKHGFLEGRHAELCFLD
VIPFWKLDLDQDYRVTCFTSWSPCFSCAQEMAKFISKNKHVSLCIFTARIYRRQGRCQEGLRTLAEAG
AKISFMTYSEFKHCWDTFVDHQGCPFQPWDGLDEHSQDLSGRLRAILQ
hAPOBEC-4 (Homo sapiens):
MEPIYEEYLANHGTIVKPYYWLSFSLDCSNCPYHIRTGEEARVSLTEFCQIFGFPYGTTFPQTKHLTF
YELKTSSGSLVQKGHASSCTGNYIHPESMLFEMNGYLDSAIYNNDSIRHIILYSNNSPCNEANHCCIS
KMYNFLITYPGITLSIYFSQLYHTEMDFPASAWNREALRSLASLWPRVVLSPISGGIWHSVLHSFISG
VSGSHVFQPILTGRALADRHNAYEINAITGVKPYFTDVLLQTKRNPNTKAQEALESYPLNNAFPGQFF
QMPSGQLQPNLPPDLRAPVVFVLVPLRDLPPMHMGQNPNKPRNIVRHLNMPQMSFQETKDLGRLPTGR
SVEIVEITEQFASSKEADEKKKKKGKK
mAPOBEC-4 (Mus musculus):
MDSLLMKQKKFLYHFKNVRWAKGRHETYLCYVVKRRDSATSCSLDFGHLRNKSGCHVELLFLRYISDW
DLDPGRCYRVTWFTSWSPCYDCARHVAEFLRWNPNLSLRIFTARLYFCEDRKAEPEGLRRLHRAGVQI
GIMTFKDYFYCWNTFVENRERTFKAWEGLHENSVRLTRQLRRILLPLYEVDDLRDAFRMLGF
rAPOBEC-4 (Rattus norvegicus):
MEPLYEEYLTHSGTIVKPYYWLSVSLNCTNCPYHIRTGEEARVPYTEFHQTFGFPWSTYPQTKHLTFY
ELRSSSGNLIQKGLASNCTGSHTHPESMLFERDGYLDSLIFHDSNIRHIILYSNNSPCDEANHCCISK
MYNFLMNYPEVILSVFFSQLYHTENQFPTSAWNREALRGLASLWPQVILSAISGGIWQSILETFVSGI
SEGLTAVRPFTAGRTLTDRYNAYEINCITEVKPYFTDALHSWQKENQDQKVWAASENQPLHNTTPAQW
QPDMSQDCRTPAVFMLVPYRDLPPIHVNPSPQKPRTVVRHLNTLQLSASKVKALRKSPSGRPVKKEEA
RKGSTRSQEANETNKSKWKKQTLFIKSNICHLLEREQKKIGILSSWSV
mfAPOBEC-4 (Macaca fascicularis):
MEPTYEEYLANHGTIVKPYYWLSFSLDCSNCPYHIRTGEEARVSLTEFCQIFGFPYGTTYPQTKHLTF
YELKTSSGSLVQKGHASSCTGNYIHPESMLFEMNGYLDSAIYNNDSIRHIILYCNNSPCNEANHCCIS
KVYNFLITYPGITLSIYFSQLYHTEMDFPASAWNREALRSLASLWPRVVLSPISGGIWHSVLHSFVSG
VSGSHVFQPILTGRALTDRYNAYEINAITGVKPFFTDVLLHTKRNPNTKAQMALESYPLNNAFPGQSF
QMTSGIPPDLRAPVVFVLLPLRDLPPMHMGQDPNKPRNIIRHLNMPQMSFQETKDLERLPTRRSVETV
EITERFASSKQAEEKTKKKKGKK
pmCDA-1 (Petromyzon marinus):
MAGYECVRVSEKLDFDTFEFQFENLHYATERHRTYVIFDVKPQSAGGRSRRLWGYIINNPNVCHAELI
LMSMIDRHLESNPGVYAMTWYMSWSPCANCSSKLNPWLKNLLEEQGHTLTMHFSRIYDRDREGDHRGL
RGLKHVSNSFRMGVVGRAEVKECLAEYVEASRRTLTWLDTTESMAAKMRRKLFCILVRCAGMRESGIP
LHLFTLQTPLLSGRVVWWRV
pmCDA-2 (Petromyzon marinus):
MELREVVDCALASCVRHEPLSRVAFLRCFAAPSQKPRGTVILFYVEGAGRGVTGGHAVNYNKQGTSIH
AEVLLLSAVRAALLRRRRCEDGEEATRGCTLHCYSTYSPCRDCVEYIQEFGASTGVRVVIHCCRLYEL
DVNRRRSEAEGVLRSLSRLGRDFRLMGPRDAIALLLGGRLANTADGESGASGNAWVTETNVVEPLVDM
TGFGDEDLHAQVQRNKQIREAYANYASAVSLMLGELHVDPDKFPFLAEFLAQTSVEPSGTPRETRGRP
RGASSRGPEIGRQRPADFERALGAYGLFLHPRIVSREADREEIKRDLIVVMRKHNYQGP
pmCDA-5 (Petromyzon marinus):
MAGDENVRVSEKLDFDTFEFQFENLHYATERHRTYVIFDVKPQSAGGRSRRLWGYIINNPNVCHAELI
LMSMIDRHLESNPGVYAMTWYMSWSPCANCSSKLNPWLKNLLEEQGHTLMMHFSRIYDRDREGDHRGL
RGLKHVSNSFRMGVVGRAEVKECLAEYVEASRRILTWLDTTESMAAKMRRKLFCILVRCAGMRESGMP
LHLFT
yCD (Saccharomyces cerevisiae):
MVTGGMASKWDQKGMDIAYEEAALGYKEGGVPIGGCLINNKDGSVLGRGHNMRFQKGSATLHGEISTL
ENCGRLEGKVYKDTTLYTTLSPCDMCTGAIIMYGIPRCVVGENVNFKSKGEKYLQTRGHEVVVVDDER
CKKIMKQFIDERPQDWFEDIGE
rAPOBEC-1 (delta 177-186):
MSSETGPVAVDPTLRRRIEPHEFEVFFDPRELRKETCLLYEINWGGRHSIWRHTSQNTNKHVEVNFIE
KFTTERYFCPNTRCSITWFLSWSPCGECSRAITEFLSRYPHVTLFIYIARLYHHADPRNRQGLRDLIS
SGVTIQIMTEQESGYMWRNFVNYSPSNEAHWPRYPHLWVRGLPPCLNILRRKQPQLITFTIALQSCHY
QRLPPHILTNATGLK
rAPOBEC-1 (delta 202-213):
MSSETGPVAVDPILRRRIEPHEFEVFFDPRELRKETCLLYEINWGGRHSIWRHTSQNTNKHVEVNFIE
KFTTERYFCPNTRCSITWFLSWSPCGECSRAITEFLSRYPHVTLFIYIARLYHHADPRNRQGLRDLIS
SGVTIQIMTEQESGYCWRNFVNYSPSNEAHWPRYPHLWVRLYVLELYCIILGLPPCLNILRRKQPQHY
QRLPPHILTNATGLK
Mouse APOBEC-3:
MGPFCLGCSHRKCYSPIRNLISQETFKFHFKNLGYAKGRKDTFLCYEVTRKDCDSPVSLHHG
VFKNKDNIHAEICFLYWFHDKVLKVLSPREEFKITWYMSWSPCFECAEQIVRFLATHHNLSL
DIFSSRLYNVQDPETQQNLCRLVQEGAQVAAMDLYEFKKCWKKFVDNGGRRFRPWKRLLTNF
RYQDSKLQEILRPCYIPVPSSSSSTLSNICLTKGLPETRFCVEGRRMDPLSEEEFYSQFYNQ
RVKHLCYYHRMKPYLCYQLEQFNGQAPLKGCLLSEKGKQHAEILFLDKIRSMELSQVTITCY
LTWSPCPNCAWQLAAFKRDRPDLILHIYTSRLYFHWKRPFQKGLCSLWQSGILVDVMDLPQF
TDCWTNFVNPKRPFWPWKGLEIISRRTQRRLRRIKESWGLQDLVNDFGNLQLGPPMS
(italic: nucleic acid editing domain)

Some aspects of the present disclosure are based on the recognition that modulating the deaminase domain catalytic activity of any of the fusion proteins described herein, for example by making point mutations in the deaminase domain, affect the processivity of the fusion proteins (e.g., base editors). For example, mutations that reduce, but do not eliminate, the catalytic activity of a deaminase domain within a base editing fusion protein can make it less likely that the deaminase domain will catalyze the deamination of a residue adjacent to a target residue, thereby narrowing the deamination window. The ability to narrow the deamination window can prevent unwanted deamination of residues adjacent to specific target residues, which can decrease or prevent off-target effects.

For example, in some embodiments, an APOBEC deaminase incorporated into a base editor can comprise one or more mutations selected from the group consisting of H121X, H122X, R126X, R126X, R118X, W90X, W90X, and R132X of rAPOBEC1, or one or more corresponding mutations in another APOBEC deaminase, wherein X is any amino acid. In some embodiments, an APOBEC deaminase incorporated into a base editor can comprise one or more mutations selected from the group consisting of H121R, H122R, R126A, R126E, R118A, W90A, W90Y, and R132E of rAPOBEC1, or one or more corresponding mutations in another APOBEC deaminase.

In some embodiments, an APOBEC deaminase incorporated into a base editor can comprise one or more mutations selected from the group consisting of D316X, D317X, R320X, R320X, R313X, W285X, W285X, R326X of hAPOBEC3G, or one or more corresponding mutations in another APOBEC deaminase, wherein X is any amino acid. In some embodiments, any of the fusion proteins provided herein comprise an APOBEC deaminase comprising one or more mutations selected from the group consisting of D316R, D317R, R320A, R320E, R313A, W285A, W285Y, R326E of hAPOBEC3G, or one or more corresponding mutations in another APOBEC deaminase.

In some embodiments, an APOBEC deaminase incorporated into a base editor can comprise a H121R and a H122R mutation of rAPOBEC1, or one or more corresponding mutations in another APOBEC deaminase. In some embodiments an APOBEC deaminase incorporated into a base editor can comprise an APOBEC deaminase comprising a R126A mutation of rAPOBEC1, or one or more corresponding mutations in another APOBEC deaminase. In some embodiments, an APOBEC deaminase incorporated into a base editor can comprise an APOBEC deaminase comprising a R126E mutation of rAPOBEC1, or one or more corresponding mutations in another APOBEC deaminase. In some embodiments, an APOBEC deaminase incorporated into a base editor can comprise an APOBEC deaminase comprising a R118A mutation of rAPOBEC1, or one or more corresponding mutations in another APOBEC deaminase. In some embodiments, an APOBEC deaminase incorporated into a base editor can comprise an APOBEC deaminase comprising a W90A mutation of rAPOBEC1, or one or more corresponding mutations in another APOBEC deaminase. In some embodiments, an APOBEC deaminase incorporated into a base editor can comprise an APOBEC deaminase comprising a W90Y mutation of rAPOBEC1, or one or more corresponding mutations in another APOBEC deaminase. In some embodiments, an APOBEC deaminase incorporated into a base editor can comprise an APOBEC deaminase comprising a R132E mutation of rAPOBEC1, or one or more corresponding mutations in another APOBEC deaminase. In some embodiments an APOBEC deaminase incorporated into a base editor can comprise an APOBEC deaminase comprising a W90Y and a R126E mutation of rAPOBEC1, or one or more corresponding mutations in another APOBEC deaminase. In some embodiments, an APOBEC deaminase incorporated into a base editor can comprise an APOBEC deaminase comprising a R126E and a R132E mutation of rAPOBEC1, or one or more corresponding mutations in another APOBEC deaminase. In some embodiments, an APOBEC deaminase incorporated into a base editor can comprise an APOBEC deaminase comprising a W90Y and a R132E mutation of rAPOBEC1, or one or more corresponding mutations in another APOBEC deaminase. In some embodiments, an APOBEC deaminase incorporated into a base editor can comprise an APOBEC deaminase comprising a W90Y, R126E, and R132E mutation of rAPOBEC1, or one or more corresponding mutations in another APOBEC deaminase.

In some embodiments, an APOBEC deaminase incorporated into a base editor can comprise an APOBEC deaminase comprising a D316R and a D317R mutation of hAPOBEC3G, or one or more corresponding mutations in another APOBEC deaminase. In some embodiments, any of the fusion proteins provided herein comprise an APOBEC deaminase comprising a R320A mutation of hAPOBEC3G, or one or more corresponding mutations in another APOBEC deaminase. In some embodiments, an APOBEC deaminase incorporated into a base editor can comprise an APOBEC deaminase comprising a R320E mutation of hAPOBEC3G, or one or more corresponding mutations in another APOBEC deaminase. In some embodiments, an APOBEC deaminase incorporated into a base editor can comprise an APOBEC deaminase comprising a R313A mutation of hAPOBEC3G, or one or more corresponding mutations in another APOBEC deaminase. In some embodiments, an APOBEC deaminase incorporated into a base editor can comprise an APOBEC deaminase comprising a W285A mutation of hAPOBEC3G, or one or more corresponding mutations in another APOBEC deaminase. In some embodiments, an APOBEC deaminase incorporated into a base editor can comprise an APOBEC deaminase comprising a W285Y mutation of hAPOBEC3G, or one or more corresponding mutations in another APOBEC deaminase. In some embodiments, an APOBEC deaminase incorporated into a base editor can comprise an APOBEC deaminase comprising a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R320E mutation of hAPOBEC3G, or one or more corresponding mutations in another APOBEC deaminase. In some embodiments, an APOBEC deaminase incorporated into a base editor can comprise an APOBEC deaminase comprising a R320E and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y and a R326E mutation of hAPOBEC3G, or one or more corresponding mutations in another APOBEC deaminase. In some embodiments, an APOBEC deaminase incorporated into a base editor can comprise an APOBEC deaminase comprising a W285Y, R320E, and R326E mutation of hAPOBEC3G, or one or more corresponding mutations in another APOBEC deaminase.

A number of modified cytidine deaminases are commercially available, including, but not limited to, SaBE3, SaKKH-BE3, VQR-BE3, EQR-BE3, VRER-BE3, YE1-BE3, EE-BE3, YE2-BE3, and YEE-BE3, which are available from Addgene (plasmids 85169, 85170, 85171, 85172, 85173, 85174, 85175, 85176, 85177). In some embodiments, a deaminase incorporated into a base editor comprises all or a portion of an APOBEC1 deaminase.

Details of C to T nucleobase editing proteins are described in International PCT Application No. PCT/US2016/058344 (WO2017/070632) and Komor, A. C., et al., “Programmable editing of a target base in genomic DNA without double-stranded DNA cleavage” Nature 533, 420-424 (2016), the entire contents of which are hereby incorporated by reference.

### Cytidine Deaminases

The fusion proteins provided herein comprise one or more cytidine deaminases. In some embodiments, the cytidine deaminases provided herein are capable of deaminating cytosine or 5-methylcytosine to uracil or thymine. In some embodiments, the cytidine deaminases provided herein are capable of deaminating cytosine in DNA. The cytidine deaminase may be derived from any suitable organism. In some embodiments, the cytidine deaminase is a naturally-occurring cytidine deaminase that includes one or more mutations corresponding to any of the mutations provided herein. One of skill in the art will be able to identify the corresponding residue in any homologous protein, e.g., by sequence alignment and determination of homologous residues. Accordingly, one of skill in the art would be able to generate mutations in any naturally-occurring cytidine deaminase that corresponds to any of the mutations described herein. In some embodiments, the cytidine deaminase is from a prokaryote. In some embodiments, the cytidine deaminase is from a bacterium. In some embodiments, the cytidine deaminase is from a mammal (e.g., human).

In some embodiments, the cytidine deaminase comprises an amino acid sequence that is at least 60%, at least 65%, at least 70%, at least 75%, at least 80%, at least 85%, at least 90%, at least 95%, at least 96%, at least 97%, at least 98%, at least 99%, or at least 99.5% identical to any one of the cytidine deaminase amino acid sequences set forth herein. It should be appreciated that cytidine deaminases provided herein may include one or more mutations (e.g., any of the mutations provided herein). The disclosure provides any deaminase domains with a certain percent identity plus any of the mutations or combinations thereof described herein. In some embodiments, the cytidine deaminase comprises an amino acid sequence that has 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or more mutations compared to a reference sequence, or any of the cytidine deaminases provided herein. In some embodiments, the cytidine deaminase comprises an amino acid sequence that has at least 5, at least 10, at least 15, at least 20, at least 25, at least 30, at least 35, at least 40, at least 45, at least 50, at least 60, at least 70, at least 80, at least 90, at least 100, at least 110, at least 120, at least 130, at least 140, at least 150, at least 160, or at least 170 identical contiguous amino acid residues as compared to any one of the amino acid sequences known in the art or described herein.

A fusion protein of the invention comprises two or more nucleic acid editing domains. In some embodiments, the nucleic acid editing domain can catalyze a C to U base change. In some embodiments, the nucleic acid editing domain is a deaminase domain. In some embodiments, the deaminase is a cytidine deaminase or an adenosine deaminase. In some embodiments, the deaminase is an apolipoprotein B mRNA-editing complex (APOBEC) family deaminase. In some embodiments, the deaminase is an APOBEC1 deaminase. In some embodiments, the deaminase is an APOBEC2 deaminase. In some embodiments, the deaminase is an APOBEC3 deaminase. In some embodiments, the deaminase is an APOBEC3 A deaminase. In some embodiments, the deaminase is an APOBEC3B deaminase. In some embodiments, the deaminase is an APOBEC3C deaminase. In some embodiments, the deaminase is an APOBEC3D deaminase. In some embodiments, the deaminase is an APOBEC3E deaminase. In some embodiments, the deaminase is an APOBEC3F deaminase. In some embodiments, the deaminase is an APOBEC3G deaminase. In some embodiments, the deaminase is an APOBEC3H deaminase. In some embodiments, the deaminase is an APOBEC4 deaminase. In some embodiments, the deaminase is an activation-induced deaminase (AID). In some embodiments, the deaminase is a vertebrate deaminase. In some embodiments, the deaminase is an invertebrate deaminase. In some embodiments, the deaminase is a human, chimpanzee, gorilla, monkey, cow, dog, rat, or mouse deaminase. In some embodiments, the deaminase is a human deaminase. In some embodiments, the deaminase is a rat deaminase, e.g., rAPOBEC1. In some embodiments, the deaminase is a Petromyzon marinus cytidine deaminase 1 (pmCDA1). In some embodiments, the deaminase is a human APOBEC3G. In some embodiments, the deaminase is a fragment of the human APOBEC3G. In some embodiments, the deaminase is a human APOBEC3G variant comprising a D316R D317R mutation. In some embodiments, the deaminase is a fragment of the human APOBEC3G and comprises mutations corresponding to the D316R D317R mutations. In some embodiments, the nucleic acid editing domain is at least 80%, at least 85%, at least 90%, at least 92%, at least 95%, at least 96%, at least 97%, at least 98%, at least 99%), or at least 99.5% identical to the deaminase domain of any deaminase described herein.

In certain embodiments, the fusion proteins provided herein comprise one or more features that improve the base editing activity of the fusion proteins. For example, any of the fusion proteins provided herein may comprise a Cas9 domain that has reduced nuclease activity. In some embodiments, any of the fusion proteins provided herein may have a Cas9 domain that does not have nuclease activity (dCas9), or a Cas9 domain that cuts one strand of a duplexed DNA molecule, referred to as a Cas9 nickase (nCas9).

Cas9 Complexes with Guide RNAs

Some aspects of this disclosure provide complexes comprising any of the fusion proteins provided herein, and a guide RNA bound to a Cas9 domain (e.g., a dCas9, a nuclease active Cas9, or a Cas9 nickase) of fusion protein. In some embodiments, the guide nucleic acid (e.g., guide RNA) is from 15-100 nucleotides long and comprises a sequence of at least 10 contiguous nucleotides that is complementary to a target sequence. In some embodiments, the guide RNA is 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 nucleotides long. In some embodiments, the guide RNA comprises a sequence of 15, 16, 17, 18, 19, 20, 21, 22, 23, 24, 25, 26, 27, 28, 29, 30, 31, 32, 33, 34, 35, 36, 37, 38, 39, or 40 contiguous nucleotides that is complementary to a target sequence. In some embodiments, the target sequence is a DNA sequence. In some embodiments, the target sequence is a sequence in the genome of a bacteria, yeast, fungi, insect, plant, or animal. In some embodiments, the target sequence is a sequence in the genome of a human. In some embodiments, the 3′ end of the target sequence is immediately adjacent to a canonical PAM sequence (NGG). In some embodiments, the 3′ end of the target sequence is immediately adjacent to a non-canonical PAM sequence (e.g., a sequence listed in Table 1 or 5′-NAA-3′). In some embodiments, the guide nucleic acid (e.g., guide RNA) is complementary to a sequence in a gene of interest (e.g., a gene in the HBV genome).

Some aspects of this disclosure provide methods of using the fusion proteins, or complexes provided herein. For example, some aspects of this disclosure provide methods comprising contacting a DNA molecule with any of the fusion proteins provided herein, and with at least one guide RNA, wherein the guide RNA is about 15-100 nucleotides long and comprises a sequence of at least 10 contiguous nucleotides that is complementary to a target sequence. In some embodiments, the 3′ end of the target sequence is immediately adjacent to an AGC, GAG, TTT, GTG, or CAA sequence. In some embodiments, the 3′ end of the target sequence is immediately adjacent to an NGA, NGCG, NGN, NNGRRT, NNNRRT, NGCG, NGCN, NGTN, NGTN, NGTN, or 5′ (TTTV) sequence.

It will be understood that the numbering of the specific positions or residues in the respective sequences depends on the particular protein and numbering scheme used. Numbering might be different, e.g., in precursors of a mature protein and the mature protein itself, and differences in sequences from species to species may affect numbering. One of skill in the art will be able to identify the respective residue in any homologous protein and in the respective encoding nucleic acid by methods well known in the art, e.g., by sequence alignment and determination of homologous residues.

It will be apparent to those of skill in the art that in order to target any of the fusion proteins disclosed herein, to a target site, e.g., a site comprising a mutation to be edited, it is typically necessary to co-express the fusion protein together with a guide RNA. As explained in more detail elsewhere herein, a guide RNA typically comprises a tracrRNA framework allowing for Cas9 binding, and a guide sequence, which confers sequence specificity to the Cas9:nucleic acid editing enzyme/domain fusion protein. Alternatively, the guide RNA and tracrRNA may be provided separately, as two nucleic acid molecules. In some embodiments, the guide RNA comprises a structure, wherein the guide sequence comprises a sequence that is complementary to the target sequence. The guide sequence is typically 20 nucleotides long. The sequences of suitable guide RNAs for targeting Cas9:nucleic acid editing enzyme/domain fusion proteins to specific genomic target sites will be apparent to those of skill in the art based on the instant disclosure. Such suitable guide RNA sequences typically comprise guide sequences that are complementary to a nucleic sequence within 50 nucleotides upstream or downstream of the target nucleotide to be edited. Some exemplary guide RNA sequences suitable for targeting any of the provided fusion proteins to specific target sequences are provided herein.

### Additional Domains

A base editor described herein can include any domain which helps to facilitate the nucleobase editing, modification or altering of a nucleobase of a polynucleotide. In some embodiments, a base editor comprises a polynucleotide programmable nucleotide binding domain (e.g., Cas9), a nucleobase editing domain (e.g., deaminase domain), and one or more additional domains. In some cases, the additional domain can facilitate enzymatic or catalytic functions of the base editor, binding functions of the base editor, or be inhibitors of cellular machinery (e.g., enzymes) that could interfere with the desired base editing result. In some embodiments, a base editor can comprise a nuclease, a nickase, a recombinase, a deaminase, a methyltransferase, a methylase, an acetylase, an acetyltransferase, a transcriptional activator, or a transcriptional repressor domain.

In some embodiments, a base editor can comprise a uracil glycosylase inhibitor (UGI) domain. A UGI domain can for example improve the efficiency of base editors comprising a cytidine deaminase domain by inhibiting the conversion of a U formed by deamination of a C back to the C nucleobase. In some cases, cellular DNA repair response to the presence of U:G heteroduplex DNA can be responsible for a decrease in nucleobase editing efficiency in cells. In such cases, uracil DNA glycosylase (UDG) can catalyze removal of U from DNA in cells, which can initiate base excision repair (BER), mostly resulting in reversion of the U:G pair to a C:G pair. In such cases, BER can be inhibited in base editors comprising one or more domains that bind the single strand, block the edited base, inhibit UGI, inhibit BER, protect the edited base, and/or promote repairing of the non-edited strand. Thus, this disclosure contemplates a base editor fusion protein comprising a UGI domain.

In some embodiments, a base editor comprises as a domain all or a portion of a double-strand break (DSB) binding protein. For example, a DSB binding protein can include a Gam protein of bacteriophage Mu that can bind to the ends of DSBs and can protect them from degradation. See Komor, A. C., et al., “Improved base excision repair inhibition and bacteriophage Mu Gam protein yields C:G-to-T:A base editors with higher efficiency and product purity” Science Advances 3:eaao4774 (2017), the entire content of which is hereby incorporated by reference.

Additionally, in some embodiments, a Gam protein can be fused to an N terminus of a base editor. In some embodiments, a Gam protein can be fused to a C-terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See. Komor, A. C., et al., “Improved base excision repair inhibition and bacteriophage Mu Gam protein yields C:G-to-T:A base editors with higher efficiency and product purity” Science Advances 3:eaao4774 (2017). In some embodiments, a mutation or mutations can change the length of a base editor domain relative to a wild-type domain. For example, a deletion of at least one amino acid in at least one domain can reduce the length of the base editor. In another case, a mutation or mutations do not change the length of a domain relative to a wild-type domain. For example, substitution(s) in any domain does/do not change the length of the base editor.

In some embodiments, a base editor can comprise as a domain all or a portion of a nucleic acid polymerase (NAP). For example, a base editor can comprise all or a portion of a eukaryotic NAP. In some embodiments, a NAP or portion thereof incorporated into a base editor is a DNA polymerase. In some embodiments, a NAP or portion thereof incorporated into a base editor has translesion polymerase activity. In some cases, a NAP or portion thereof incorporated into a base editor is a translesion DNA polymerase. In some embodiments, a NAP or portion thereof incorporated into a base editor is a Rev7, Rev1 complex, polymerase iota, polymerase kappa, or polymerase eta. In some embodiments, a NAP or portion thereof incorporated into a base editor is a eukaryotic polymerase alpha, beta, gamma, delta, epsilon, gamma, eta, iota, kappa, lambda, mu, or nu component. In some embodiments, a NAP or portion thereof incorporated into a base editor comprises an amino acid sequence that is at least 75%, 80%, 85%, 90%, 95%, 96%, 97%, 98%, 99%, or 99.5% identical to a nucleic acid polymerase (e.g., a translesion DNA polymerase).

### Base Editor System

The base editor system provided herein comprises the steps of: (a) contacting a target nucleotide sequence of a polynucleotide (e.g., a double-stranded DNA or RNA, a single-stranded DNA or RNA) of a subject with a base editor system comprising an adenosine deaminase domain or a cytidine deaminase domain, wherein the aforementioned domains are fused to a polynucleotide binding domain, thereby forming a nucleobase editor capable of inducing changes at one or more bases within a nucleic acid molecule as described herein and at least one guide polynucleic acid (e.g., gRNA), wherein the target nucleotide sequence comprises a targeted nucleobase pair; (b) inducing strand separation of the target region; (c) converting a first nucleobase of the target nucleobase pair in a single strand of the target region to a second nucleobase; and (d) cutting no more than one strand of the target region, where a third nucleobase complementary to the first nucleobase base is replaced by a fourth nucleobase complementary to the second nucleobase. It should be appreciated that in some embodiments, step (b) is omitted. In some embodiments, the targeted nucleobase pair is a plurality of nucleobase pairs in one or more genes. In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more genes, wherein at least one gene is located in a different locus.

In some embodiments, the cut single strand (nicked strand) is hybridized to the guide nucleic acid. In some embodiments, the cut single strand is opposite to the strand comprising the first nucleobase. In some embodiments, the base editor comprises a Cas9 domain. In some embodiments, the first base is adenine, and the second base is not a G, C, A, or T. In some embodiments, the second base is inosine.

Base editing system as provided herein provides a new approach to genome editing that uses a fusion protein containing a catalytically defective Streptococcus pyogenes Cas9, a cytidine deaminase, and an inhibitor of base excision repair to induce programmable, single nucleotide (C→T or A→G) changes in DNA without generating double-strand DNA breaks, without requiring a donor DNA template, and without inducing an excess of stochastic insertions and deletions.

Provided herein are systems, compositions, and methods for editing a nucleobase using a base editor system. In some embodiments, the base editor system comprises a base editor (BE) comprising a polynucleotide programmable nucleotide binding domain and a nucleobase editing domain (e.g., deaminase domain) for editing the nucleobase; and a guide polynucleotide (e.g., guide RNA) in conjunction with the polynucleotide programmable nucleotide binding domain. In some embodiments, the base editor system comprises a base editor (BE) comprising a polynucleotide programmable nucleotide binding domain and a nucleobase editing domain (e.g., deaminase domain) for editing the nucleobase, and a guide polynucleotide (e.g., guide RNA) in conjunction with the polynucleotide programmable nucleotide binding domain. In some embodiments, the polynucleotide programmable nucleotide binding domain is a polynucleotide programmable DNA binding domain. In some embodiments, the polynucleotide programmable nucleotide binding domain is a polynucleotide programmable RNA binding domain. In some cases, a deaminase domain can be a cytosine deaminase or a cytidine deaminase, an adenine deaminase or an adenosine deaminase. In some embodiments, the terms “cytosine deaminase” and “cytidine deaminase” can be used interchangeably. In some embodiments, the terms “adenine deaminase” and “adenosine deaminase” can be used interchangeably. In some cases, a deaminase domain can be a cytosine deaminase or a cytidine deaminase. In some cases, a deaminase domain can be an adenine deaminase or an adenosine deaminase.

Details of nucleobase editing proteins are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, a single guide polynucleotide may be utilized to target a deaminase to a target nucleic acid sequence. In some embodiments, a single pair of guide polynucleotides may be utilized to target different deaminases to a target nucleic acid sequence. The nucleobase components and the polynucleotide programmable nucleotide binding component of a base editor system may be associated with each other covalently or non-covalently. For example, in some embodiments, the deaminase domain can be targeted to a target nucleotide sequence by a polynucleotide programmable nucleotide binding domain. In some embodiments, a polynucleotide programmable nucleotide binding domain can be fused or linked to a deaminase domain. In some embodiments, a polynucleotide programmable nucleotide binding domain can target a deaminase domain to a target nucleotide sequence by non-covalently interacting with or associating with the deaminase domain. For example, in some embodiments, the nucleobase editing component, e.g., the deaminase component can comprise an additional heterologous portion or domain that is capable of interacting with, associating with, or capable of forming a complex with an additional heterologous portion or domain that is part of a polynucleotide programmable nucleotide binding domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a steril alpha motif, a telomerase Ku binding motif and Ku protein, a telomerase Sm7 binding motif and Sm7 protein, or a RNA recognition motif.

A base editor system may further comprise a guide polynucleotide component. It should be appreciated that components of the base editor system may be associated with each other via covalent bonds, noncovalent interactions, or any combination of associations and interactions thereof. In some embodiments, a deaminase domain can be targeted to a target nucleotide sequence by a guide polynucleotide. For example, in some embodiments, the nucleobase editing component of the base editor system, e.g., the deaminase component, can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain (e.g., polynucleotide binding domain such as an RNA or DNA binding protein) can be fused or linked to the deaminase domain. In some embodiments, the additional heterologous portion may be capable of binding to, interacting with, associating with, or forming a complex with a polypeptide. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif.

In some embodiments, a base editor system can further comprise an inhibitor of base excision repair (BER) component. It should be appreciated that components of the base editor system may be associated with each other via covalent bonds, noncovalent interactions, or any combination of associations and interactions thereof. The inhibitor of BER component may comprise a base excision repair inhibitor. In some embodiments, the inhibitor of base excision repair can be a uracil DNA glycosylase inhibitor (UGI). In some embodiments, the inhibitor of base excision repair can be an inosine base excision repair inhibitor. In some embodiments, the inhibitor of base excision repair can be targeted to the target nucleotide sequence by the polynucleotide programmable nucleotide binding domain. In some embodiments, a polynucleotide programmable nucleotide binding domain can be fused or linked to an inhibitor of base excision repair. In some embodiments, a polynucleotide programmable nucleotide binding domain can be fused or linked to a deaminase domain and an inhibitor of base excision repair. In some embodiments, a polynucleotide programmable nucleotide binding domain can target an inhibitor of base excision repair to a target nucleotide sequence by non-covalently interacting with or associating with the inhibitor of base excision repair. For example, in some embodiments, the inhibitor of base excision repair component can comprise an additional heterologous portion or domain that is capable of interacting with, associating with, or capable of forming a complex with an additional heterologous portion or domain that is part of a polynucleotide programmable nucleotide binding domain. In some embodiments, the inhibitor of base excision repair can be targeted to the target nucleotide sequence by the guide polynucleotide. For example, in some embodiments, the inhibitor of base excision repair can comprise an additional heterologous portion or domain (e.g., polynucleotide binding domain such as an RNA or DNA binding protein) that is capable of interacting with, associating with, or capable of forming a complex with a portion or segment (e.g., a polynucleotide motif) of a guide polynucleotide. In some embodiments, the additional heterologous portion or domain of the guide polynucleotide (e.g., polynucleotide binding domain such as an RNA or DNA binding protein) can be fused or linked to the inhibitor of base excision repair. In some embodiments, the additional heterologous portion may be capable of binding to, interacting with, associating with, or forming a complex with a polynucleotide. In some embodiments, the additional heterologous portion may be capable of binding to a guide polynucleotide. In some embodiments, the additional heterologous portion may be capable of binding to a polypeptide linker. In some embodiments, the additional heterologous portion may be capable of binding to a polynucleotide linker. The additional heterologous portion may be a protein domain. In some embodiments, the additional heterologous portion may be a K Homology (KH) domain, a MS2 coat protein domain, a PP7 coat protein domain, a SfMu Com coat protein domain, a sterile alpha motif, a telomerase Ku binding motif and Ku protein, a telomerase Sm7 binding motif and Sm7 protein, or a RNA recognition motif.

In some embodiments, the base editor inhibits base excision repair of the edited strand. In some embodiments, the base editor protects or binds the non-edited strand. In some embodiments, the base editor comprises UGI activity. In some embodiments, the base editor comprises a catalytically inactive inosine-specific nuclease. In some embodiments, the base editor comprises nickase activity. In some embodiments, the intended edit of base pair is upstream of a PAM site. In some embodiments, the intended edit of base pair is 1, 2, 3, 4, 5, 6, 7, 8, 9, 10, 11, 12, 13, 14, 15, 16, 17, 18, 19, or 20 nucleotides upstream of the PAM site. In some embodiments, the intended edit of base-pair is downstream of a PAM site. In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site.

In some embodiments, the method does not require a canonical (e.g., NGG) PAM site. In some embodiments, the nucleobase editor comprises a linker or a spacer. In some embodiments, the linker or spacer is 1-25 amino acids in length. In some embodiments, the linker or spacer is 5-20 amino acids in length. In some embodiments, the linker or spacer is 10, 11, 12, 13, 14, 15, 16, 17, 18, 19, or 20 amino acids in length.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window.

In some embodiments, non-limiting exemplary cytidine base editors (CBE) include BE1 (APOBEC1-XTEN-dCas9), BE2 (APOBEC1-XTEN-dCas9-UGI), BE3 (APOBEC1-XTEN-dCas9(A840H)-UGI), BE3-Gam, saBE3, saBE4-Gam, BE4, BE4-Gam, saBE4, or saB4E-Gam. BE4 extends the APOBEC1-Cas9n(D10A) linker to 32 amino acids and the Cas9n-UGI linker to 9 amino acids, and appends a second copy of UGI to the C-terminus of the construct with another 9-amino acid linker into a single base editor construct. The base editors saBE3 and saBE4 have the S. pyogenes Cas9n(D10A) replaced with the smaller S. aureus Cas9n(D10A). BE3-Gam, saBE3-Gam, BE4-Gam, and saBE4-Gam have 174 residues of Gam protein fused to the N-terminus of BE3, saBE3, BE4, and saBE4 via the 16 amino acid XTEN linker.

In some embodiments, the adenosine base editor (ABE) can deaminate adenine in DNA. In some embodiments, ABE is generated by replacing APOBEC1 component of BE3 with natural or engineered E. coli TadA, human ADAR2, mouse ADA, or human ADAT2. In some embodiments, ABE comprises an evolved TadA variant. In some embodiments, the ABE is ABE 1.2 (TadA*-XTEN-nCas9-NLS). In some embodiments, TadA* comprises A106V and D108N mutations.

In some embodiments, the ABE is a second-generation ABE. In some embodiments, the ABE is ABE2.1, which comprises additional mutations D147Y and E155V in TadA* (TadA*2.1). In some embodiments, the ABE is ABE2.2, ABE2.1 fused to catalytically inactivated version of human alkyl adenine DNA glycosylase (AAG with E125Q mutation). In some embodiments, the ABE is ABE2.3, ABE2.1 fused to catalytically inactivated version of E. coli Endo V (inactivated with D35A mutation). In some embodiments, the ABE is ABE2.6 which has a linker twice as long (32 amino acids, (SGGS)₂-XTEN-(SGGS)₂) as the linker in ABE2.1. In some embodiments, the ABE is ABE2.7, which is ABE2.1 tethered with an additional wild-type TadA monomer. In some embodiments, the ABE is ABE2.8, which is ABE2.1 tethered with an additional TadA*2.1 monomer. In some embodiments, the ABE is ABE2.9, which is a direct fusion of evolved TadA (TadA*2.1) to the N-terminus of ABE2.1. In some embodiments, the ABE is ABE2.10, which is a direct fusion of wild type TadA to the N-terminus of ABE2.1. In some embodiments, the ABE is ABE2.11, which is ABE2.9 with an inactivating E59A mutation at the N-terminus of TadA* monomer. In some embodiments, the ABE is ABE2.12, which is ABE2.9 with an inactivating E59A mutation in the internal TadA* monomer.

In some embodiments, the ABE is a third generation ABE. In some embodiments, the ABE is ABE3.1, which is ABE2.3 with three additional TadA mutations (L84F, H123Y, and I157F).

In some embodiments, the ABE is a fourth generation ABE. In some embodiments, the ABE is ABE4.3, which is ABE3.1 with an additional TadA mutation A142N (TadA*4.3).

In some embodiments, the ABE is a fifth generation ABE. In some embodiments, the ABE is ABE5.1, which is generated by importing a consensus set of mutations from surviving clones (H36L, R51L, S146C, and K157N) into ABE3.1. In some embodiments, the ABE is ABE5.3, which has a heterodimeric construct containing wild-type E. coli TadA fused to an internal evolved TadA*. In some embodiments, the ABE is ABE5.2, ABE5.4, ABE5.5, ABE5.6, ABE5.7, ABE5.8, ABE5.9, ABE5.10, ABE5.11, ABE5.12, ABE5.13, or ABE5.14, as shown in below Table 7. In some embodiments, the ABE is a sixth generation ABE. In some embodiments, the ABE is ABE6.1, ABE6.2, ABE6.3, ABE6.4, ABE6.5, or ABE6.6, as shown in below Table 7. In some embodiments, the ABE is a seventh generation ABE. In some embodiments, the ABE is ABE7.1, ABE7.2, ABE7.3, ABE7.4, ABE7.5, ABE7.6, ABE7.7, ABE7.8, ABE 7.9, or ABE7.10, as shown in Table 7 below.

TABLE 7
Genotypes of ABEs

| | 23 | 26 | 36 | 37 | 48 | 49 | 51 | 72 | 84 | 87 | 106 | 108 | 123 | 125 | 142 | 146 | 147 | 152 | 155 | 156 | 157 | 161 |
|---|---|---|---|---|---|---|---|---|---|---|---|---|---|---|---|---|---|---|---|---|---|---|
| ABE0.1 | W | R | H | N | P | | R | N | L | S | A | D | H | G | A | S | D | R | E | I | K | K |
| ABE0.2 | W | R | H | N | P | | R | N | L | S | A | D | H | G | A | S | D | R | E | I | K | K |
| ABE1.1 | W | R | H | N | P | | R | N | L | S | A | N | H | G | A | S | D | R | E | I | K | K |
| ABE1.2 | W | R | H | N | P | | R | N | L | S | V | N | H | G | A | S | D | R | E | I | K | K |
| ABE2.1 | W | R | H | N | P | | R | N | L | S | V | N | H | G | A | S | Y | R | V | I | K | K |
| ABE2.2 | W | R | H | N | P | | R | N | L | S | V | N | H | G | A | S | Y | R | V | I | K | K |
| ABE2.3 | W | R | H | N | P | | R | N | L | S | V | N | H | G | A | S | Y | R | V | I | K | K |
| ABE2.4 | W | R | H | N | P | | R | N | L | S | V | N | H | G | A | S | Y | R | V | I | K | K |
| ABE2.5 | W | R | H | N | P | | R | N | L | S | V | N | H | G | A | S | Y | R | V | I | K | K |
| ABE2.6 | W | R | H | N | P | | R | N | L | S | V | N | H | G | A | S | Y | R | V | I | K | K |
| ABE2.7 | W | R | H | N | P | | R | N | L | S | V | N | H | G | A | S | Y | R | V | I | K | K |
| ABE2.8 | W | R | H | N | P | | R | N | L | S | V | N | H | G | A | S | Y | R | V | I | K | K |
| ABE2.9 | W | R | H | N | P | | R | N | L | S | V | N | H | G | A | S | Y | R | V | I | K | K |
| ABE2.10 | W | R | H | N | P | | R | N | L | S | V | N | H | G | A | S | Y | R | V | I | K | K |
| ABE2.11 | W | R | H | N | P | | R | N | L | S | V | N | H | G | A | S | Y | R | V | I | K | K |
| ABE2.12 | W | R | H | N | P | | R | N | L | S | V | N | H | G | A | S | Y | R | V | I | K | K |
| ABE3.1 | W | R | H | N | P | | R | N | F | S | V | N | Y | G | A | S | Y | R | V | F | K | K |
| ABE3.2 | W | R | H | N | P | | R | N | F | S | V | N | Y | G | A | S | Y | R | V | F | K | K |
| ABE3.3 | W | R | H | N | P | | R | N | F | S | V | N | Y | G | A | S | Y | R | V | F | K | K |
| ABE3.4 | W | R | H | N | P | | R | N | F | S | V | N | Y | G | A | S | Y | R | V | F | K | K |
| ABE3.5 | W | R | H | N | P | | R | N | F | S | V | N | Y | G | A | S | Y | R | V | F | K | K |
| ABE3.6 | W | R | H | N | P | | R | N | F | S | V | N | Y | G | A | S | Y | R | V | F | K | K |
| ABE3.7 | W | R | H | N | P | | R | N | F | S | V | N | Y | G | A | S | Y | R | V | F | K | K |
| ABE3.8 | W | R | H | N | P | | R | N | F | S | V | N | Y | G | A | S | Y | R | V | F | K | K |
| ABE4.1 | W | R | H | N | P | | R | N | L | S | V | N | H | G | N | S | Y | R | V | I | K | K |
| ABE4.2 | W | G | H | N | P | | R | N | L | S | V | N | H | G | N | S | Y | R | V | I | K | K |
| ABE4.3 | W | R | H | N | P | | R | N | F | S | V | N | Y | G | N | S | Y | R | V | F | K | K |
| ABE5.1 | W | R | L | N | P | | L | N | F | S | V | N | Y | G | A | C | Y | R | V | F | N | K |
| ABE5.2 | W | R | H | S | P | | R | N | F | S | V | N | Y | G | A | S | Y | R | V | F | K | T |
| ABE5.3 | W | R | L | N | P | | L | N | I | S | V | N | Y | G | A | C | Y | R | V | F | N | K |
| ABE5.4 | W | R | H | S | P | | R | N | F | S | V | N | Y | G | A | S | Y | R | V | F | K | T |
| ABE5.5 | W | R | L | N | P | | L | N | F | S | V | N | Y | G | A | C | Y | R | V | F | N | K |
| ABE5.6 | W | R | L | N | P | | L | N | F | S | V | N | Y | G | A | C | Y | R | V | F | N | K |
| ABE5.7 | W | R | L | N | P | | L | N | F | S | V | N | Y | G | A | C | Y | R | V | F | N | K |
| ABE5.8 | W | R | L | N | P | | L | N | F | S | V | N | Y | G | A | C | Y | R | V | F | N | K |
| ABE5.9 | W | R | L | N | P | | L | N | F | S | V | N | Y | G | A | C | Y | R | V | F | N | K |
| ABE5.10 | W | R | L | N | P | | L | N | F | S | V | N | Y | G | A | C | Y | R | V | F | N | K |
| ABE5.11 | W | R | L | N | P | | L | N | F | S | V | N | Y | G | A | C | Y | R | V | F | N | K |
| ABE5.12 | W | R | L | N | P | | L | N | F | S | V | N | Y | G | A | C | Y | R | V | F | N | K |
| ABE5.13 | W | R | H | N | P | | L | D | F | S | V | N | Y | A | A | S | Y | R | V | F | K | K |
| ABE5.14 | W | R | H | N | S | | L | N | F | C | V | N | Y | G | A | S | Y | R | V | F | K | K |
| ABE6.1 | W | R | H | N | S | | L | N | F | S | V | N | Y | G | N | S | Y | R | V | F | K | K |
| ABE6.2 | W | R | H | N | T | V | L | N | F | S | V | N | Y | G | N | S | Y | R | V | F | N | K |
| ABE6.3 | W | R | L | N | S | | L | N | F | S | V | N | Y | G | A | C | Y | R | V | F | N | K |
| ABE6.4 | W | R | L | N | S | | L | N | F | S | V | N | Y | G | N | C | Y | R | V | F | N | K |
| ABE6.5 | W | R | L | N | T | V | L | N | F | S | V | N | Y | G | A | C | Y | R | V | F | N | K |
| ABE6.6 | W | R | L | N | T | V | L | N | F | S | V | N | Y | G | N | C | Y | R | V | F | N | K |
| ABE7.1 | W | R | L | N | A | | L | N | F | S | V | N | Y | G | A | C | Y | R | V | F | N | K |
| ABE7.2 | W | R | L | N | A | | L | N | F | S | V | N | Y | G | N | C | Y | R | V | F | N | K |
| ABE7.3 | L | R | L | N | A | | L | N | F | S | V | N | Y | G | A | C | Y | R | V | F | N | K |
| ABE7.4 | R | R | L | N | A | | L | N | F | S | V | N | Y | G | A | C | Y | R | V | F | N | K |
| ABE7.5 | W | R | L | N | A | | L | N | F | S | V | N | Y | G | A | C | Y | H | V | F | N | K |
| ABE7.6 | W | R | L | N | A | | L | N | I | S | V | N | Y | G | A | C | Y | P | V | F | N | K |
| ABE7.7 | L | R | L | N | A | | L | N | F | S | V | N | Y | G | A | C | Y | P | V | F | N | K |
| ABE7.8 | L | R | L | N | A | | L | N | F | S | V | N | Y | G | N | C | Y | R | V | F | N | K |
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

In some embodiments, the base editor is an eighth generation ABE (ABE8). In some embodiments, the ABE8 contains a TadA*8 variant. In some embodiments, the ABE8 has a monomeric construct containing a TadA*8 variant (“ABE8.x-m”). In some embodiments, the ABE8 is ABE8.1-m, which has a monomeric construct containing TadA*7.10 with a Y147T mutation (TadA*8.1). In some embodiments, the ABE8 is ABE8.2-m, which has a monomeric construct containing TadA*7.10 with a Y147R mutation (TadA*8.2). In some embodiments, the ABE8 is ABE8.3-m, which has a monomeric construct containing TadA*7.10 with a Q154S mutation (TadA*8.3). In some embodiments, the ABE8 is ABE8.4-m, which has a monomeric construct containing TadA*7.10 with a Y123H mutation (TadA*8.4). In some embodiments, the ABE8 is ABE8.5-m, which has a monomeric construct containing TadA*7.10 with a V82S mutation (TadA*8.5). In some embodiments, the ABE8 is ABE8.6-m, which has a monomeric construct containing TadA*7.10 with a T166R mutation (TadA*8.6). In some embodiments, the ABE8 is ABE8.7-m, which has a monomeric construct containing TadA*7.10 with a Q154R mutation (TadA*8.7). In some embodiments, the ABE8 is ABE8.8-m, which has a monomeric construct containing TadA*7.10 with Y147R, Q154R, and Y123H mutations (TadA*8.8). In some embodiments, the ABE8 is ABE8.9-m, which has a monomeric construct containing TadA*7.10 with Y147R, Q154R and I76Y mutations (TadA*8.9). In some embodiments, the ABE8 is ABE8.10-m, which has a monomeric construct containing TadA*7.10 with Y147R, Q154R, and T166R mutations (TadA*8.10). In some embodiments, the ABE8 is ABE8.11-m, which has a monomeric construct containing TadA*7.10 with Y147T and Q154R mutations (TadA*8.11). In some embodiments, the ABE8 is ABE8.12-m, which has a monomeric construct containing TadA*7.10 with Y147T and Q154S mutations (TadA*8.12). In some embodiments, the ABE8 is ABE8.13-m, which has a monomeric construct containing TadA*7.10 with Y123H (Y123H reverted from H123Y), Y147R, Q154R and I76Y mutations (TadA*8.13). In some embodiments, the ABE8 is ABE8.14-m, which has a monomeric construct containing TadA*7.10 with I76Y and V82S mutations (TadA*8.14). In some embodiments, the ABE8 is ABE8.15-m, which has a monomeric construct containing TadA*7.10 with V82S and Y147R mutations (TadA*8.15). In some embodiments, the ABE8 is ABE8.16-m, which has a monomeric construct containing TadA*7.10 with V82S, Y123H (Y123H reverted from H123Y) and Y147R mutations (TadA*8.16). In some embodiments, the ABE8 is ABE8.17-m, which has a monomeric construct containing TadA*7.10 with V82S and Q154R mutations (TadA*8.17). In some embodiments, the ABE8 is ABE8.18-m, which has a monomeric construct containing TadA*7.10 with V82S, Y123H (Y123H reverted from H123Y) and Q154R mutations (TadA*8.18). In some embodiments, the ABE8 is ABE8.19-m, which has a monomeric construct containing TadA*7.10 with V82S, Y123H (Y123H reverted from H123Y), Y147R and Q154R mutations (TadA*8.19). In some embodiments, the ABE8 is ABE8.20-m, which has a monomeric construct containing TadA*7.10 with I76Y, V82S, Y123H (Y123H reverted from H123Y), Y147R and Q154R mutations (TadA*8.20). In some embodiments, the ABE8 is ABE8.21-m, which has a monomeric construct containing TadA*7.10 with Y147R and Q154S mutations (TadA*8.21). In some embodiments, the ABE8 is ABE8.22-m, which has a monomeric construct containing TadA*7.10 with V82S and Q154S mutations (TadA*8.22). In some embodiments, the ABE8 is ABE8.23-m, which has a monomeric construct containing TadA*7.10 with V82S and Y123H (Y123H reverted from H123Y) mutations (TadA*8.23). In some embodiments, the ABE8 is ABE8.24-m, which has a monomeric construct containing TadA*7.10 with V82S, Y123H (Y123H reverted from H123Y), and Y147T mutations (TadA*8.24).

In some embodiments, the ABE8 has a heterodimeric construct containing wild-type E. coli TadA fused to a TadA*8 variant (“ABE8.x-d”). In some embodiments, the ABE8 is ABE8.1-d, which has a heterodimeric construct containing wild-type E. coli TadA fused to TadA*7.10 with a Y147T mutation (TadA*8.1). In some embodiments, the ABE8 is ABE8.2-d, which has a heterodimeric construct containing wild-type E. coli TadA fused to TadA*7.10 with a Y147R mutation (TadA*8.2). In some embodiments, the ABE8 is ABE8.3-d, which has a heterodimeric construct containing wild-type E. coli TadA fused to TadA*7.10 with a Q154S mutation (TadA*8.3). In some embodiments, the ABE8 is ABE8.4-d, which has a heterodimeric construct containing wild-type E. coli TadA fused to TadA*7.10 with a Y123H mutation (TadA*8.4). In some embodiments, the ABE8 is ABE8.5-d, which has a heterodimeric construct containing wild-type E. coli TadA fused to TadA*7.10 with a V82S mutation (TadA*8.5). In some embodiments, the ABE8 is ABE8.6-d, which has a heterodimeric construct containing wild-type E. coli TadA fused to TadA*7.10 with a T166R mutation (TadA*8.6). In some embodiments, the ABE8 is ABE8.7-d, which has a heterodimeric construct containing wild-type E. coli TadA fused to TadA*7.10 with a Q154R mutation (TadA*8.7). In some embodiments, the ABE8 is ABE8.8-d, which has a heterodimeric construct containing wild-type E. coli TadA fused to TadA*7.10 with Y147R, Q154R, and Y123H mutations (TadA*8.8). In some embodiments, the ABE8 is ABE8.9-d, which has a heterodimeric construct containing wild-type E. coli TadA fused to TadA*7.10 with Y147R, Q154R and I76Y mutations (TadA*8.9). In some embodiments, the ABE8 is ABE8.10-d, which has a heterodimeric construct containing wild-type E. coli TadA fused to TadA*7.10 with Y147R, Q154R, and T166R mutations (TadA*8.10). In some embodiments, the ABE8 is ABE8.11-d, which has a heterodimeric construct containing wild-type E. coli TadA fused to TadA*7.10 with Y147T and Q154R mutations (TadA*8.11). In some embodiments, the ABE8 is ABE8.12-d, which has heterodimeric construct containing wild-type E. coli TadA fused to TadA*7.10 with Y147T and Q154S mutations (TadA*8.12). In some embodiments, the ABE8 is ABE8.13-d, which has a heterodimeric construct containing wild-type E. coli TadA fused to TadA*7.10 with Y123H (Y123H reverted from H123Y), Y147R, Q154R and I76Y mutations (TadA*8.13). In some embodiments, the ABE8 is ABE8.14-d, which has a heterodimeric construct containing wild-type E. coli TadA fused to TadA*7.10 with I76Y and V82S mutations (TadA*8.14). In some embodiments, the ABE8 is ABE8.15-d, which has a heterodimeric construct containing wild-type E. coli TadA fused to TadA*7.10 with V82S and Y147R mutations (TadA*8.15). In some embodiments, the ABE8 is ABE8.16-d, which has a heterodimeric construct containing wild-type E. coli TadA fused to TadA*7.10 with V82S, Y123H (Y123H reverted from H123Y) and Y147R mutations (TadA*8.16). In some embodiments, the ABE8 is ABE8.17-d, which has a heterodimeric construct containing wild-type E. coli TadA fused to TadA*7.10 with V82S and Q154R mutations (TadA*8.17). In some embodiments, the ABE8 is ABE8.18-d, which has a heterodimeric construct containing wild-type E. coli TadA fused to TadA*7.10 with V82S, Y123H (Y123H reverted from H123Y) and Q154R mutations (TadA*8.18). In some embodiments, the ABE8 is ABE8.19-d, which has a heterodimeric construct containing wild-type E. coli TadA fused to TadA*7.10 with V82S, Y123H (Y123H reverted from H123Y), Y147R and Q154R mutations (TadA*8.19). In some embodiments, the ABE8 is ABE8.20-d, which has a heterodimeric construct containing wild-type E. coli TadA fused to TadA*7.10 with I76Y, V82S, Y123H (Y123H reverted from H123Y), Y147R and Q154R mutations (TadA*8.20). In some embodiments, the ABE8 is ABE8.21-d, which has a heterodimeric construct containing wild-type E. coli TadA fused to TadA*7.10 with Y147R and Q154S mutations (TadA*8.21). In some embodiments, the ABE8 is ABE8.22-d, which has a heterodimeric construct containing wild-type E. coli TadA fused to TadA*7.10 with V82S and Q154S mutations (TadA*8.22). In some embodiments, the ABE8 is ABE8.23-d, which has a heterodimeric construct containing wild-type E. coli TadA fused to TadA*7.10 with V82S and Y123H (Y123H reverted from H123Y) mutations (TadA*8.23). In some embodiments, the ABE8 is ABE8.24-d, which has a heterodimeric construct containing wild-type E. coli TadA fused to TadA*7.10 with V82S, Y123H (Y123H reverted from H123Y), and Y147T mutations (TadA*8.24).

In some embodiments, the ABE8 has a heterodimeric construct containing TadA*7.10 fused to a TadA*8 variant (“ABE8.x-7”). In some embodiments, the ABE8 is ABE8.1-7, which has a heterodimeric construct containing TadA*7.10 fused to TadA*7.10 with a Y147T mutation (TadA*8.1). In some embodiments, the ABE8 is ABE8.2-7, which has a heterodimeric construct containing TadA*7.10 fused to TadA*7.10 with a Y147R mutation (TadA*8.2). In some embodiments, the ABE8 is ABE8.3-7, which has a heterodimeric construct containing TadA*7.10 fused to TadA*7.10 with a Q154S mutation (TadA*8.3). In some embodiments, the ABE8 is ABE8.4-7, which has a heterodimeric construct containing TadA*7.10 fused to TadA*7.10 with a Y123H mutation (TadA*8.4). In some embodiments, the ABE8 is ABE8.5-7, which has a heterodimeric construct containing TadA*7.10 fused to TadA*7.10 with a V82S mutation (TadA*8.5). In some embodiments, the ABE8 is ABE8.6-7, which has a heterodimeric construct containing TadA*7.10 fused to TadA*7.10 with a T166R mutation (TadA*8.6). In some embodiments, the ABE8 is ABE8.7-7, which has a heterodimeric construct containing TadA*7.10 fused to TadA*7.10 with a Q154R mutation (TadA*8.7). In some embodiments, the ABE8 is ABE8.8-7, which has a heterodimeric construct containing TadA*7.10 fused to TadA*7.10 with Y147R, Q154R, and Y123H mutations (TadA*8.8). In some embodiments, the ABE8 is ABE8.9-7, which has a heterodimeric construct containing TadA*7.10 fused to TadA*7.10 with Y147R, Q154R and I76Y mutations (TadA*8.9). In some embodiments, the ABE8 is ABE8.10-7, which has a heterodimeric construct containing TadA*7.10 fused to TadA*7.10 with Y147R, Q154R, and T166R mutations (TadA*8.10). In some embodiments, the ABE8 is ABE8.11-7, which has a heterodimeric construct containing TadA*7.10 fused to TadA*7.10 with Y147T and Q154R mutations (TadA*8.11). In some embodiments, the ABE8 is ABE8.12-7, which has a heterodimeric construct containing TadA*7.10 fused to TadA*7.10 with Y147T and Q154S mutations (TadA*8.12). In some embodiments, the ABE8 is ABE8.13-7, which has a heterodimeric construct containing TadA*7.10 fused to TadA*7.10 with Y123H (Y123H reverted from H123Y), Y147R, Q154R and I76Y mutations (TadA*8.13). In some embodiments, the ABE8 is ABE8.14-7, which has a heterodimeric construct containing TadA*7.10 fused to TadA*7.10 with I76Y and V82S mutations (TadA*8.14). In some embodiments, the ABE8 is ABE8.15-7, which has a heterodimeric construct containing TadA*7.10 fused to TadA*7.10 with V82S and Y147R mutations (TadA*8.15). In some embodiments, the ABE8 is ABE8.16-7, which has a heterodimeric construct containing TadA*7.10 fused to TadA*7.10 with V82S, Y123H (Y123H reverted from H123Y) and Y147R mutations (TadA*8.16). In some embodiments, the ABE8 is ABE8.17-7, which has a heterodimeric construct containing TadA*7.10 fused to TadA*7.10 with V82S and Q154R mutations (TadA*8.17). In some embodiments, the ABE8 is ABE8.18-7, which has a heterodimeric construct containing TadA*7.10 fused to TadA*7.10 with V82S, Y123H (Y123H reverted from H123Y) and Q154R mutations (TadA*8.18). In some embodiments, the ABE8 is ABE8.19-7, which has a heterodimeric construct containing TadA*7.10 fused to TadA*7.10 with V82S, Y123H (Y123H reverted from H123Y), Y147R and Q154R mutations (TadA*8.19). In some embodiments, the ABE8 is ABE8.20-7, which has a heterodimeric construct containing TadA*7.10 fused to TadA*7.10 with I76Y, V82S, Y123H (Y123H reverted from H123Y), Y147R and Q154R mutations (TadA*8.20). In some embodiments, the ABE8 is ABE8.21-7, which has a heterodimeric construct containing TadA*7.10 fused to TadA*7.10 with Y147R and Q154S mutations (TadA*8.21). In some embodiments, the ABE8 is ABE8.22-7, which has a heterodimeric construct containing TadA*7.10 fused to TadA*7.10 with V82S and Q154S mutations (TadA*8.22). In some embodiments, the ABE8 is ABE8.23-7, which has a heterodimeric construct containing TadA*7.10 fused to TadA*7.10 with V82S and Y123H (Y123H reverted from H123Y) mutations (TadA*8.23). In some embodiments, the ABE8 is ABE8.24-7, which has a heterodimeric construct containing TadA*7.10 fused to TadA*7.10 with V82S, Y123H (Y123H reverted from H123Y), and Y147T mutations (TadA*8.24

In some embodiments, the ABE is ABE8.1-m, ABE8.2-m, ABE8.3-m, ABE8.4-m, ABE8.5-m, ABE8.6-m, ABE8.7-m, ABE8.8-m, ABE8.9-m, ABE8.10-m, ABE8.11-m, ABE8.12-m, ABE8.13-m, ABE8.14-m, ABE8.15-m, ABE8.16-m, ABE8.17-m, ABE8.18-m, ABE8.19-m, ABE8.20-m, ABE8.21-m, ABE8.22-m, ABE8.23-m, ABE8.24-m, ABE8.1-d, ABE8.2-d, ABE8.3-d, ABE8.4-d, ABE8.5-d, ABE8.6-d, ABE8.7-d, ABE8.8-d, ABE8.9-d, ABE8.10-d, ABE8.11-d, ABE8.12-d, ABE8.13-d, ABE8.14-d, ABE8.15-d, ABE8.16-d, ABE8.17-d, ABE8.18-d, ABE8.19-d, ABE8.20-d, ABE8.21-d, ABE8.22-d, ABE8.23-d, or ABE8.24-d as shown in Table 8 below.

TABLE 8
Adenosine Deaminase Base Editor 8 Variants

| | Adenosine | |
|---|---|---|
| ABE8 | Deaminase | Adenosine Deaminase Description |
| ABE8.1-m | TadA* 8.1 | Monomer_TadA* 7.10 + Y147T |
| ABE8.2-m | TadA* 8.2 | Monomer_TadA* 7.10 + Y147R |
| ABE8.3-m | TadA* 8.3 | Monomer_TadA* 7.10 + Q154S |
| ABE8.4-m | TadA* 8.4 | Monomer_TadA* 7.10 + Y123H |
| ABE8.5-m | TadA* 8.5 | Monomer_TadA* 7.10 + V82S |
| ABE8.6-m | TadA* 8.6 | Monomer_TadA* 7.10 + T166R |
| ABE8.7-m | TadA* 8.7 | Monomer_TadA* 7.10 + Q154R |
| ABE8.8-m | TadA* 8.8 | Monomer_TadA* 7.10 + Y147R_Q154R_Y123H |
| ABE8.9-m | TadA* 8.9 | Monomer_TadA* 7.10 + Y147R_Q154R_176Y |
| ABE8.10-m | TadA* 8.10 | Monomer_TadA* 7.10 + Y147R_Q154R_T166R |
| ABE8.11-m | TadA* 8.11 | Monomer_TadA* 7.10 + Y147T_Q154R |
| ABE8.12-m | TadA* 8.12 | Monomer_TadA*7.10 + Y147T_Q154S |
| ABE8.13-m | TadA* 8.13 | Monomer_TadA* 7.10 + Y123H_Y147R_Q154R_176Y |
| ABE8.14-m | TadA* 8.14 | Monomer_TadA* 7.10 + I76Y_V82S |
| ABE8.15-m | TadA* 8.15 | Monomer_TadA*7.10 + V82S_Y147R |
| ABE8.16-m | TadA* 8.16 | Monomer TadA* 7.10 + V82S Y123H Y147R |
| ABE8.17-m | TadA* 8.17 | Monomer_TadA* 7.10 + V82S_Q154R |
| ABE8.18-m | TadA* 8.18 | Monomer_TadA* 7.10 + V82S Y123H_Q154R |
| ABE8.19-m | TadA* 8.19 | Monomer_TadA* 7.10 + V82S_Y123H_Y147R_Q154R |
| ABE8.20-m | TadA* 8.20 | Monomer_TadA* 7.10 + I76Y_V82S_Y123H_Y147R_Q154R |
| ABE8.21-m | TadA* 8.21 | Monomer_TadA* 7.10 + Y147R_Q 154S |
| ABE8.22-m | TadA* 8.22 | Monomer_TadA* 7.10 + V82S_Q154S |
| ABE8.23-m | TadA* 8.23 | Monomer TadA* 7.10 + V82S Y123H |
| ABE8.24-m | TadA* 8.24 | Monomer_TadA* 7.10 + V82S_Y123H_Y147T |
| ABE8.1-d | TadA*8.1 | Heterodimer_(WT) + (TadA* 7.10 + Y147T) |
| ABE8.2-d | TadA* 8.2 | Heterodimer_(WT) + (TadA* 7.10 + Y147R) |
| ABE8.3-d | TadA* 8.3 | Heterodimer_(WT) + (TadA* 7.10 + Q154 S) |
| ABE8.4-d | TadA* 8.4 | Heterodimer (WT) + (TadA*7.10 + Y123H) |
| ABE8.5-d | TadA* 8.5 | Heterodimer_(WT) + (TadA* 7.10 + V82S) |
| ABE8.6-d | TadA* 8.6 | Heterodimer_(WT) + (TadA* 7.10 + T166R) |
| ABE8.7-d | TadA* 8.7 | Heterodimer_(WT) + (TadA* 7.10 + Q154R) |
| ABE8.8-d | TadA* 8.8 | Heterodimer_(WT) + (TadA* 7.10 + Y147R_Q154R_Y123H) |
| ABE8.9-d | TadA* 8.9 | Heterodimer (WT) + (TadA* 7.10 + Y147R_Q154R_176Y) |
| ABE8.10-d | TadA* 8.10 | Heterodimer_(WT) + (TadA*7.10 + Y147R_Q154R_T166R) |
| ABE8.11-d | TadA* 8.11 | Heterodimer_(WT) + (TadA* 7.10 + Y147T_Q154R) |
| ABE8.12-d | TadA* 8.12 | Heterodimer_(WT) + (TadA*7.10 + Y147T_Q154S) |
| ABE8.13-d | TadA* 8.13 | Heterodimer_(WT) + (TadA*7.10 + Y123H_Y147T_Q154R_176Y) |
| ABE8.14-d | TadA*8.14 | Heterodimer_(WT) + (TadA* 7.10 + I76Y_V82S) |
| ABE8.15-d | TadA* 8.15 | Heterodimer_(WT) + (TadA* 7.10 + V82S_ Y147R) |
| ABE8.16-d | TadA* 8.16 | Heterodimer_(WT) + (TadA* 7.10 + V82S_Y123H_Y147R) |
| ABE8.17-d | TadA* 8.17 | Heterodimer_(WT) + (TadA* 7.10 + V82S_Q154R) |
| ABE8.18-d | TadA* 8.18 | Heterodimer_(WT) + (TadA* 7.10 + V82S_Y123H_Q154R) |
| ABE8.19-d | TadA* 8.19 | Heterodimer_(WT) + (TadA* 7.10 + V82S_Y123H_Y147R_Q154R) |
| ABE8.20-d | TadA* 8.20 | Heterodimer (WT) + (TadA* 7.10 + |
| | | I76Y V82S Y123H Y147R Q154R) |
| ABE8.21-d | TadA* 8.21 | Heterodimer_(WT) + (TadA* 7.10 + Y147R_Q154S) |
| ABE8.22-d | TadA* 8.22 | Heterodimer_(WT) + (TadA* 7.10 + V82S_Q154S) |
| ABE8.23-d | TadA* 8.23 | Heterodimer_(WT) + (TadA*7.10 + V82S_Y123H) |
| ABE8.24-d | TadA* 8.24 | Heterodimer (WT) + (TadA* 7.10 + V82S_Y123H_Y147T) |

In some embodiments, base editors (e.g., ABE8) are generated by cloning an adenosine deaminase variant (e.g., TadA*8) into a scaffold that includes a circular permutant Cas9 (e.g., CP5 or CP6) and a bipartite nuclear localization sequence. In some embodiments, the base editor (e.g., ABE7.9, ABE7.10, or ABE8) is an NGC PAM CP5 variant (S. pyrogenes Cas9 or spVRQR Cas9). In some embodiments, the base editor (e.g., ABE7.9, ABE7.10, or ABE8) is an AGA PAM CP5 variant (S. pyrogenes Cas9 or spVRQR Cas9). In some embodiments, the base editor (e.g., ABE7.9, ABE7.10, or ABE8) is an NGC PAM CP6 variant (S. pyrogenes Cas9 or spVRQR Cas9). In some embodiments, the base editor (e.g. ABE7.9, ABE7.10, or ABE8) is an AGA PAM CP6 variant (S. pyrogenes Cas9 or spVRQR Cas9).

In some embodiments, the ABE has a genotype as shown in Table 9 below.

TABLE 9
Genotypes of ABEs

| | 23 | 26 | 36 | 37 | 48 | 49 | 51 | 72 | 84 | 87 | 105 | 108 | 123 | 125 | 142 | 145 | 147 | 152 | 155 | 156 | 157 | 161 |
|---|---|---|---|---|---|---|---|---|---|---|---|---|---|---|---|---|---|---|---|---|---|---|
| ABE7.9 | L | R | L | N | A | | L | N | F | S | V | N | Y | G | N | C | Y | P | V | F | N | K |
| ABE7.10 | R | R | L | N | A | | L | N | F | S | V | N | Y | G | A | C | Y | P | V | F | N | K |

As shown in Table 10 below, genotypes of 40 ABE8s are described. Residue positions in the evolved E. coli TadA portion of ABE are indicated. Mutational changes in ABE8 are shown when distinct from ABE7.10 mutations. In some embodiments, the ABE has a genotype of one of the ABEs as shown in Table 10 below.

TABLE 10
Residue Identity in Evolved TadA

| | 23 | 36 | 48 | 51 | 76 | 82 | 84 | 106 | 108 | 123 | 146 | 147 | 152 | 154 | 155 | 156 | 157 | 166 |
|---|---|---|---|---|---|---|---|---|---|---|---|---|---|---|---|---|---|---|
| ABE7.10 | R | L | A | L | I | V | F | V | N | Y | C | Y | P | Q | V | F | N | T |
| ABE8.1-m | | | | | | | | | | | | T | | | | | | |
| ABE8.2-m | | | | | | | | | | | | R | | | | | | |
| ABE8.3-m | | | | | | | | | | | | S | | | | | | |
| ABE8.4-m | | | | | | | | H | | | | | | | | | | |
| ABE8.5-m | | | | S | | | | | | | | | | | | | | |
| ABE8.6-m | | | | | | | | | | | | | | | | | | R |
| ABE8.7-m | | | | | | | | | | | | R | | | | | | |
| ABE8.8-m | | | | | | | | H | | R | | R | | | | | | |
| ABE8.9-m | | | Y | | | | | | | R | | R | | | | | | |
| ABE8.10-m | | | | | | | | | | R | | R | | | | | | R |
| ABE8.11-m | | | | | | | | | | T | | R | | | | | | |
| ABE8.12-m | | | | | | | | | | T | | S | | | | | | |
| ABE8.13-m | | | Y | | | | | H | | R | | R | | | | | | |
| ABE8.14-m | | | Y | S | | | | | | | | | | | | | | |
| ABE8.15-m | | | | S | | | | | | R | | | | | | | | |
| ABE8.16-m | | | | S | | | | H | | R | | | | | | | | |
| ABE8.17-m | | | | S | | | | | | | | R | | | | | | |
| ABE8.18-m | | | | S | | | | H | | | | R | | | | | | |
| ABE8.19-m | | | | S | | | | H | | R | | R | | | | | | |
| ABE8.20-m | | | Y | S | | | | H | | R | | R | | | | | | |
| ABE8.21-m | | | | | | | | | | R | | S | | | | | | |
| ABE8.22-m | | | | S | | | | | | | | S | | | | | | |
| ABE8.23-m | | | | S | | | | H | | | | | | | | | | |
| ABE8.24-m | | | | S | | | | H | | T | | | | | | | | |
| ABE8.1-d | | | | | | | | | | T | | | | | | | | |
| ABE8.2-d | | | | | | | | | | R | | | | | | | | |
| ABE8.3-d | | | | | | | | | | | | S | | | | | | |
| ABE8.4-d | | | | | | | | H | | | | | | | | | | |
| ABE8.5-d | | | | S | | | | | | | | | | | | | | |
| ABE8.6-d | | | | | | | | | | | | | | | | | | R |
| ABE8.7-d | | | | | | | | | | | | R | | | | | | |
| ABE8.8-d | | | | | | | | H | | R | | R | | | | | | |
| ABE8.9-d | | | Y | | | | | | | R | | R | | | | | | |
| ABE8.10-d | | | | | | | | | | R | | R | | | | | | R |
| ABE8.11-d | | | | | | | | | | T | | R | | | | | | |
| ABE8.12-d | | | | | | | | | | T | | S | | | | | | |
| ABE8.13-d | | | Y | | | | | H | | R | | R | | | | | | |
| ABE8.14-d | | | Y | S | | | | | | | | | | | | | | |
| ABE8.15-d | | | | S | | | | | | R | | | | | | | | |
| ABE8.16-d | | | | S | | | | H | | R | | | | | | | | |
| ABE8.17-d | | | | S | | | | | | | | R | | | | | | |
| ABE8.18-d | | | | S | | | | H | | | | R | | | | | | |
| ABE8.19-d | | | | S | | | | H | | R | | R | | | | | | |
| ABE8.20-d | | | Y | S | | | | H | | R | | R | | | | | | |
| ABE8.21-d | | | | | | | | | | R | | S | | | | | | |
| ABE8.22-d | | | | S | | | | | | | | S | | | | | | |
| ABE8.23-d | | | | S | | | | H | | | | | | | | | | |
| ABE8.24-d | | | | S | | | | H | | T | | | | | | | | |

In some embodiments, the base editor is ABE8.1, which comprises or consists essentially of the following sequence or a fragment thereof having adenosine deaminase activity:

ABE8.1_Y147T_CP5_NGC PAM_monomer
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCTFFRMPRQVFNAQKKAQSSTD [symbol] [symbol]
EIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFMQPT
VAYSVLVVAKVEKGKSKKLKSVKELLGITIMERSSFEKNPIDFLEAKGYKEVKKDLIIKLPK
YSLFELENGRKRMLASAKFLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPRAF
KYFDTTIARKEYRSTKEVLDATLIHQSITGLYETRIDLSQLGGD [symbol] [symbol]
DKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEGADKRTADGSEFESPKKKRKV*

In the above sequence, the plain text denotes an adenosine deaminase sequence, bold sequence indicates sequence derived from Cas9, the italics sequence denotes a linker sequence, and the underlined sequence denotes a bipartite nuclear localization sequence.

In some embodiments, the base editor is ABE8.1, which comprises or consists essentially of the following sequence or a fragment thereof having adenosine deaminase activity:

pNMG-B335 ABE8.1_Y147T_CP5_NGC PAM_monomer:
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCTFFRMPRQVFNAQKKAQSSTDSGGSSGGSSGSETPGTSES
ATPESSGGSSGGSEIGKATAKYFFYSNIMNFFKTEITLANGEIRKRPLIETNGETGEIVWDK
GRDFATVRKVLSMPQVNIVKKTEVQTGGFSKESILPKRNSDKLIARKKDWDPKKYGGFMQPT
VAYSVLVVAKVEKGKSKKLKSVKELLGITIMERSSFEKNPIDFLEAKGYKEVKKDLIIKLPK
YSLFELENGRKRMLASAKFLQKGNELALPSKYVNFLYLASHYEKLKGSPEDNEQKQLFVEQH
KHYLDEIIEQISEFSKRVILADANLDKVLSAYNKHRDKPIREQAENIIHLFTLTNLGAPRAF
KYFDTTIARKEYRSTKEVLDATLIHQSITGLYETRIDLSQLGGDGGSGGSGGSGGSGGSGGS
GGMDKKYSIGLAIGTNSVGWAVITDEYKVPSKKFKVLGNTDRHSIKKNLIGALLFDSGETAE
ATRLKRTARRRYTRRKNRICYLQEIFSNEMAKVDDSFFHRLEESFLVEEDKKHERHPIFGNI
VDEVAYHEKYPTIYHLRKKLVDSTDKADLRLIYLALAHMIKFRGHFLIEGDLNPDNSDVDKL
FIQLVQTYNQLFEENPINASGVDAKAILSARLSKSRRLENLIAQLPGEKKNGLFGNLIALSL
GLTPNFKSNFDLAEDAKLQLSKDTYDDDLDNLLAQIGDQYADLFLAAKNLSDAILLSDILRV
NTEITKAPLSASMIKRYDEHHQDLTLLKALVRQQLPEKYKEIFFDQSKNGYAGYIDGGASQE
EFYKFIKPILEKMDGTEELLVKLNREDLLRKQRTFDNGSIPHQIHLGELHAILRRQEDFYPF
LKDNREKIEKILTFRIPYYVGPLARGNSRFAWMTRKSEETITPWNFEEVVDKGASAQSFIER
MTNFDKNLPNEKVLPKHSLLYEYFTVYNELTKVKYVTEGMRKPAFLSGEQKKAIVDLLFKTN
RKVTVKQLKEDYFKKIECFDSVEISGVEDRFNASLGTYHDLLKIIKDKDFLDNEENEDILED
IVLTLTLFEDREMIEERLKTYAHLFDDKVMKQLKRRRYTGWGRLSRKLINGIRDKQSGKTIL
DFLKSDGFANRNFMQLIHDDSLTFKEDIQKAQVSGQGDSLHEHIANLAGSPAIKKGILQTVK
VVDELVKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRSDKNRGK
SDNVPSEEVVKKMKNYWRQLLNAKLITQRKFDNLTKAERGGLSELDKAGFIKRQLVETRQIT
KHVAQILDSRMNTKYDENDKLIREVKVITLKSKLVSDFRKDFQFYKVREINNYHHAHDAYLN
AVVGTALIKKYPKLESEFVYGDYKVYDVRKMIAKSEQEGADKRTADGSEFESPKKKRKV*

In the above sequence, the plain text denotes an adenosine deaminase sequence, bold sequence indicates sequence derived from Cas9, the italics sequence denotes a linker sequence, and the underlined sequence denotes a bipartite nuclear localization sequence.

In some embodiments, the base editor is ABE8.14, which comprises or consists essentially of the following sequence or a fragment thereof having adenosine deaminase activity:

pNMG-357_ABE8.14_with_NGC PAM CP5
MSEVEFSHEYWMRHALTLAKRAWDEREVPVGAVLVHNNRVIGEGWNRPIGRHDPTAHAEIMA
LRQGGLVMQNYRLIDATLYVTLEPCVMCAGAMIHSRIGRVVFGARDAKTGAAGSLMDVLHHP
GMNHRVEITEGILADECAALLSDFFRMRRQEIKAQKKAQSSTDGGSSGGSSGSETPGTSESA
TPESSGGSSGGSMSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIGRVVFGVRNAKTG
AAGSLMDVLHYPGMNHRVEITEGILADECAALLCTFFRMPRQVFNAQKKAQSSTDSGGSSGG
SSGSETPGTSESATPESSGGSSGGSEIGKATAKYFFYSNIMNFFKTEITLANGEIRKRPLIE
TNGETGEIVWDKGRDFATVRKVLSMPQVNIVKKTEVQTGGFSKESILPKRNSDKLIARKKDW
DPKKYGGFMQPTVAYSVLVVAKVEKGKSKKLKSVKELLGITIMERSSFEKNPIDFLEAKGYK
EVKKDLIIKLPKYSLFELENGRKRMLASAKFLQKGNELALPSKYVNFLYLASHYEKLKGSPE
DNEQKQLFVEQHKHYLDEIIEQISEFSKRVILADANLDKVLSAYNKHRDKPIREQAENIIHL
FTLTNLGAPRAFKYFDTTIARKEYRSTKEVLDATLIHQSITGLYETRIDLSQLGGDGGSGGS
GGSGGSGGSGGSGGMDKKYSIGLAIGTNSVGWAVITDEYKVPSKKFKVLGNTDRHSIKKNLI
GALLFDSGETAEATRLKRTARRRYTRRKNRICYLQEIFSNEMAKVDDSFFHRLEESFLVEED
KKHERHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLPGEKK
NGLFGNLIALSLGLTPNFKSNFDLAEDAKLQLSKDTYDDDLDNLLAQIGDQYADLFLAAKNL
SDAILLSDILRVNTEITKAPLSASMIKRYDEHHQDLTLLKALVRQQLPEKYKEIFFDQSKNG
YAGYIDGGASQEEFYKFIKPILEKMDGTEELLVKLNREDLLRKQRTFDNGSIPHQIHLGELH
AILRRQEDFYPFLKDNREKIEKILTFRIPYYVGPLARGNSRFAWMTRKSEETITPWNFEEVV
DKGASAQSFIERMTNFDKNLPNEKVLPKHSLLYEYFTVYNELTKVKYVTEGMRKPAFLSGEQ
KKAIVDLLFKTNRKVTVKQLKEDYFKKIECFDSVEISGVEDRFNASLGTYHDLLKIIKDKDF
LDNEENEDILEDIVLTLTLFEDREMIEERLKTYAHLFDDKVMKQLKRRRYTGWGRLSRKLIN
GIRDKQSGKTILDFLKSDGFANRNFMQLIHDDSLTFKEDIQKAQVSGQGDSLHEHIANLAGS
PAIKKGILQTVKVVDELVKVMGRHKPENIVIEMARENQTTQKGQKNSRERMKRIEEGIKELG
SQILKEHPVENTQLQNEKLYLYYLQNGRDMYVDQELDINRLSDYDVDHIVPQSFLKDDSIDN
KVLTRSDKNRGKSDNVPSEEVVKKMKNYWRQLLNAKLITQRKFDNLTKAERGGLSELDKAGF
IKRQLVETRQITKHVAQILDSRMNTKYDENDKLIREVKVITLKSKLVSDFRKDFQFYKVREI
NNYHHAHDAYLNAVVGTALIKKYPKLESEFVYGDYKVYDVRKMIAKSEQEGADKRTADGSEF
ESPKKKRKV*

In the above sequence, the plain text denotes an adenosine deaminase sequence, bold sequence indicates sequence derived from Cas9, the italics sequence denotes a linker sequence, and the underlined sequence denotes a bipartite nuclear localization sequence.

In some embodiments, the base editor is ABE8.8-m, which comprises or consists essentially of the following sequence or a fragment thereof having adenosine deaminase activity.

ABE8.8-m
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGL [symbol] IGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AlLLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.8-d, which comprises or consists essentially of the following sequence or a fragment thereof having adenosine deaminase activity:

ABE8.8-d
mSEVEFSHEYWMRHALTLAKRAWDEREVPVGAVLVHNNRVIGEGWNRPIGRHDPTAHAEIMA
LRQGGLVMQNYRLIDATLYVTLEPCVMCAGAMIHSRIGRVVFGARDAKTGAAGSLMDVLHHP
GMNHRVEITEGILADECAALLSDFFRMRRQEIKAQKKAQSSTDSGGSSGGSSGSETPGTSES
ATPESSGGSSGGSSEVEFSHEYWMRHALTLAKRARDEREVPVGAVLVLNNRVIGEGWNRAIG
LHDPTAHAEIMALRQGGLVMQNYRLIDATLYVTFEPCVMCAGAMIHSRIGRVVFGVRNAKTG
AAGSLMDVLHHPGMNHRVEITEGILADECAALLCRFFRMPRRVFNAQKKAQSSTDSGGSSGG
SSGSETPGTSESATPESSGGSSGGSDKKYSIGL [symbol] IGTNSVGWAVITDEYKVPSKKFKVLGNT
DRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFDTTIDRKRYTSTK
EVLDATLIHQSITGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.13-m, which comprises or consists essentially of the following sequence or a fragment thereof having adenosine deaminase activity:

ABE8.13-m
MSEVEFSHEYWMRHALTLAKRARDEREVPVGAVLVLNNRVIGEGWNRAIGLHDPTAHAEIMA
LRQGGLVMQNYRLYDATLYVTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGL [symbol] IGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AlLLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.13-d, which comprises or consists essentially of the following sequence or a fragment thereof having adenosine deaminase activity.

ABE8.13-d
MSEVEFSHEYWMRHALTLAKRAWDEREVPVGAVLVHNNRVIGEGWNRPIGRHDPTAHAEIMA
LRQGGLVMQNYRLIDATLYVTLEPCVMCAGAMIHSRIGRVVFGARDAKTGAAGSLMDVLHHP
GMNHRVEITEGILADECAALLSDFFRMRRQEIKAQKKAQSSTDSGGSSGGSSGSETPGTSES
ATPESSGGSSGGSSEVEFSHEYWMRHALTLAKRARDEREVPVGAVLVLNNRVIGEGWNRAIG
LHDPTAHAEIMALRQGGLVMQNYRLYDATLYVTFEPCVMCAGAMIHSRIGRVVFGVRNAKTG
AAGSLMDVLHHPGMNHRVEITEGILADECAALLCRFFRMPRRVFNAQKKAQSSTDSGGSSGG
SSGSETPGTSESATPESSGGSSGGSDKKYSIGL [symbol] IGTNSVGWAVITDEYKVPSKKFKVLGNT
DRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFDTTIDRKRYTSTK
EVLDATLIHQSITGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.17-m, which comprises or consists essentially of the following sequence or a fragment thereof having adenosine deaminase activity:

ABE8.17-m
MSEVEFSHEYWMRHALTLAKRARDEREVPVGAVLVLNNRVIGEGWNRAIGLHDPTAHAEIMA
LRQGGLVMQNYRLIDATLYSTFEPCVMCAGAMIHSRIGRVVFGVRNAKTGAAGSLMDVLHYP
GMNHRVEITEGILADECAALLCYFFRMPRRVFNAQKKAQSSTDSGGSSGGSSGSETPGTSES
ATPESSGGSSGGSDKKYSIGL [symbol] IGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.17-d, which comprises or consists essentially of the following sequence or a fragment thereof having adenosine deaminase activity:

ABE8.17-d
MSEVEFSHEYWMRHALTLAKRAWDEREVPVGAVLVHNNRVIGEGWNRPIGRHDPTAHAEIMA
LRQGGLVMQNYRLIDATLYVTLEPCVMCAGAMIHSRIGRVVFGARDAKTGAAGSLMDVLHHP
GMNHRVEITEGILADECAALLSDFFRMRRQEIKAQKKAQSSTDSGGSSGGSSGSETPGTSES
ATPESSGGSSGGSSEVEFSHEYWMRHALTLAKRARDEREVPVGAVLVLNNRVIGEGWNRAIG
LHDPTAHAEIMALRQGGLVMQNYRLIDATLYSTFEPCVMCAGAMIHSRIGRVVFGVRNAKTG
AAGSLMDVLHYPGMNHRVEITEGILADECAALLCYFFRMPRRVFNAQKKAQSSTDSGGSSGG
SSGSETPGTSESATPESSGGSSGGSDKKYSIGL [symbol] IGTNSVGWAVITDEYKVPSKKFKVLGNT
DRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFDTTIDRKRYTSTK
EVLDATLIHQSITGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.20-m, which comprises or consists essentially of the following sequence or a fragment thereof having adenosine deaminase activity:

ABE8.20-m
MSEVEFSHEYWMRHALTLAKRARDEREVPVGAVLVLNNRVIGEGWNRAIGLHDPTAHAEIMA
LRQGGLVMQNYRLYDATLYSTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGL [symbol] IGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFDSPTVAYSVLVVAKVEKGKSKKLKSVKELLGI
TIMERSSFEKNPIDFLEAKGYKEVKKDLIIKLPKYSLFELENGRKRMLASAGELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKRYTSTKEVLDATLIHQSI
TGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, the base editor is ABE8.20-d, which comprises or consists essentially of the following sequence or a fragment thereof having adenosine deaminase activity:

ABE8.20-d
MSEVEFSHEYWMRHALTLAKRAWDEREVPVGAVLVHNNRVIGEGWNRPIGRHDPTAHAEIMA
LRQGGLVMQNYRLIDATLYVTLEPCVMCAGAMIHSRIGRVVFGARDAKTGAAGSLMDVLHHP
GMNHRVEITEGILADECAALLSDFFRMRRQEIKAQKKAQSSTDSGGSSGGSSGSETPGTSES
ATPESSGGSSGGSSEVEFSHEYWMRHALTLAKRARDEREVPVGAVLVLNNRVIGEGWNRAIG
LHDPTAHAEIMALRQGGLVMQNYRLYDATLYSTFEPCVMCAGAMIHSRIGRVVFGVRNAKTG
AAGSLMDVLHHPGMNHRVEITEGILADECAALLCRFFRMPRRVFNAQKKAQSSTDSGGSSGG
SSGSETPGTSESATPESSGGSSGGSDKKYSIGL [symbol] IGTNSVGWAVITDEYKVPSKKFKVLGNT
DRHSIKKNLIGALLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKADLRLIYLALAHMI
KFRGHFLIEGDLNPDNSDVDKLFIQLVQTYNQLFEENPINASGVDAKAILSARLSKSRRLEN
LIAQLPGEKKNGLFGNLIALSLGLTPNFKSNFDLAEDAKLQLSKDTYDDDLDNLLAQIGDQY
ADLFLAAKNLSDAILLSDILRVNTEITKAPLSASMIKRYDEHHQDLTLLKALVRQQLPEKYK
EIFFDQSKNGYAGYIDGGASQEEFYKFIKPILEKMDGTEELLVKLNREDLLRKQRTFDNGSI
PHQIHLGELHAILRRQEDFYPFLKDNREKIEKILTFRIPYYVGPLARGNSRFAWMTRKSEET
ITPWNFEEVVDKGASAQSFIERMTNFDKNLPNEKVLPKHSLLYEYFTVYNELTKVKYVTEGM
RKPAFLSGEQKKAIVDLLFKTNRKVTVKQLKEDYFKKIECFDSVEISGVEDRFNASLGTYHD
LLKIIKDKDFLDNEENEDILEDIVLTLTLFEDREMIEERLKTYAHLFDDKVMKQLKRRRYTG
WGRLSRKLINGIRDKQSGKTILDFLKSDGFANRNFMQLIHDDSLTFKEDIQKAQVSGQGDSL
HEHIANLAGSPAIKKGILQTVKVVDELVKVMGRHKPENIVIEMARENQTTQKGQKNSRERMK
RIEEGIKELGSQILKEHPVENTQLQNEKLYLYYLQNGRDMYVDQELDINRLSDYDVDHIVPQ
SFLKDDSIDNKVLTRSDKNRGKSDNVPSEEVVKKMKNYWRQLLNAKLITQRKFDNLTKAERG
GLSELDKAGFIKRQLVETRQITKHVAQILDSRMNTKYDENDKLIREVKVITLKSKLVSDFRK
DFQFYKVREINNYHHAHDAYLNAVVGTALIKKYPKLESEFVYGDYKVYDVRKMIAKSEQEIG
KATAKYFFYSNIMNFFKTEITLANGEIRKRPLIETNGETGEIVWDKGRDFATVRKVLSMPQV
NIVKKTEVQTGGFSKESILPKRNSDKLIARKKDWDPKKYGGFDSPTVAYSVLVVAKVEKGKS
KKLKSVKELLGITIMERSSFEKNPIDFLEAKGYKEVKKDLIIKLPKYSLFELENGRKRMLAS
AGELQKGNELALPSKYVNFLYLASHYEKLKGSPEDNEQKQLFVEQHKHYLDEIIEQISEFSK
RVILADANLDKVLSAYNKHRDKPIREQAENIIHLFTLTNLGAPAAFKYFDTTIDRKRYTSTK
EVLDATLIHQSITGLYETRIDLSQLGGDEGADKRTADGSEFESPKKKRKV*

In the above sequence, the plain text denotes an adenosine deaminase sequence, bold sequence indicates sequence derived from Cas9, the italicized sequence denotes a linker sequence, underlined sequence denotes a bipartite nuclear localization sequence, and double underlined sequence indicates mutations.

In some embodiments, a ABE8 of the invention is selected from the following sequences:

01. monoABE8.1_bpNLS + Y147T
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCTFFRMPRQVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
02. monoABE8.1_bpNLS + Y147R
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCRFFRMPRQVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
03. monoABE8.1_bpNLS + Q154S
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCYFFRMPRSVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
04. monoABE8.1_bpNLS + Y123H
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHHP
GMNHRVEITEGILADECAALLCYFFRMPRQVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
05. monoABE8.1_bpNLS + V82S
MSEVEFSHEYWMRHALTLAKRARDEREVPVGAVLVLNNRVIGEGWNRAIGLHDPTAHAEIMA
LRQGGLVMQNYRLIDATLYSTFEPCVMCAGAMIHSRIGRVVFGVRNAKTGAAGSLMDVLHYP
GMNHRVEITEGILADECAALLCYFFRMPRQVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
6. monoABE8.1_bpNLS + T166R
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCYFFRMPRQVFNAQKKAQSSRDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
7. monoABE8.1_bpNLS + Q154R
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCYFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
08. monoABE8.1_bpNLS + Y147R_Q154R_Y123H
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
09. monoABE8.1_bpNLS + Y147R_Q154R_I76Y
MSEVEFSHEYWMRHALTLAKRARDEREVPVGAVLVLNNRVIGEGWNRAIGLHDPTAHAEIMA
LRQGGLVMQNYRLYDATLYVTFEPCVMCAGAMIHSRIGRVVFGVRNAKTGAAGSLMDVLHYP
GMNHRVEITEGILADECAALLCRFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
10. monoABE8.1_bpNLS + Y147R_Q154R_T166R
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCRFFRMPRRVFNAQKKAQSSRDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
11. monoABE8.1_bpNLS + Y147T_Q154R
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCIFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
12. monoABE8.1_bpNLS + Y147T_Q1545
MSEVEFSHEYWMRHALTLAKRARDEREVPVGAVLVLNNRVIGEGWNRAIGLHDPTAHAEIMA
LRQGGLVMQNYRLIDATLYVTFEPCVMCAGAMIHSRIGRVVFGVRNAKTGAAGSLMDVLHYP
GMNHRVEITEGILADECAALLCIFFRMPRSVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
13. monoABE8.1_bpNLS + H123Y123H_Y147R_Q154R_I76Y
MSEVEFSHEYWMRHALTLAKRARDEREVPVGAVLVLNNRVIGEGWNRAIGLHDPTAHAEIMA
LRQGGLVMQNYRLYDATLYVTFEPCVMCAGAMIHSRIGRVVFGVRNAKTGAAGSLMDVLHHP
GMNHRVEITEGILADECAALLCRFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV
14. monoABE8.1_bpNLS + V82S + Q154R
MSEVEFSHEYWMRHALTLAKRARDEREVPVGAVLVLNNRVIGEGWNRAIGLHDPTAHAEIMA
LRQGGLVMQNYRLIDATLYSTFEPCVMCAGAMIHSRIGRVVFGVRNAKTGAAGSLMDVLHYP
GMNHRVEITEGILADECAALLCYFFRMPRRVFNAQKKAQSSTDSGGSSGGSSGSETPGTSES
ATPESSGGSSGGSDKKYSIGLAIGTNSVGWAVITDEYKVPSKKFKVLGNTDRHSIKKNLIGA
LLFDSGETAEATRLKRTARRRYTRRKNRICYLQEIFSNEMAKVDDSFFHRLEESFLVEEDKK
HERHPIFGNIVDEVAYHEKYPTIYHLRKKLVDSTDKADLRLIYLALAHMIKFRGHFLIEGDL
NPDNSDVDKLFIQLVQTYNQLFEENPINASGVDAKAILSARLSKSRRLENLIAQLPGEKKNG
LFGNLIALSLGLTPNFKSNFDLAEDAKLQLSKDTYDDDLDNLLAQIGDQYADLFLAAKNLSD
AILLSDILRVNTEITKAPLSASMIKRYDEHHQDLTLLKALVRQQLPEKYKEIFFDQSKNGYA
GYIDGGASQEEFYKFIKPILEKMDGTEELLVKLNREDLLRKQRTFDNGSIPHQIHLGELHAI
LRRQEDFYPFLKDNREKIEKILTFRIPYYVGPLARGNSRFAWMTRKSEETITPWNFEEVVDK
GASAQSFIERMTNFDKNLPNEKVLPKHSLLYEYFTVYNELTKVKYVTEGMRKPAFLSGEQKK
AIVDLLFKTNRKVTVKQLKEDYFKKIECFDSVEISGVEDRFNASLGTYHDLLKIIKDKDFLD
NEENEDILEDIVLTLTLFEDREMIEERLKTYAHLFDDKVMKQLKRRRYTGWGRLSRKLINGI
RDKQSGKTILDFLKSDGFANRNFMQLIHDDSLTFKEDIQKAQVSGQGDSLHEHIANLAGSPA
IKKGILQTVKVVDELVKVMGRHKPENIVIEMARENQTTQKGQKNSRERMKRIEEGIKELGSQ
ILKEHPVENTQLQNEKLYLYYLQNGRDMYVDQELDINRLSDYDVDHIVPQSFLKDDSIDNKV
LTRSDKNRGKSDNVPSEEVVKKMKNYWRQLLNAKLITQRKFDNLTKAERGGLSELDKAGFIK
RQLVETRQITKHVAQILDSRMNTKYDENDKLIREVKVITLKSKLVSDFRKDFQFYKVREINN
YHHAHDAYLNAVVGTALIKKYPKLESEFVYGDYKVYDVRKMIAKSEQEIGKATAKYFFYSNI
MNFFKTEITLANGEIRKRPLIETNGETGEIVWDKGRDFATVRKVLSMPQVNIVKKTEVQTGG
FSKESILPKRNSDKLIARKKDWDPKKYGGFVSPTVAYSVLVVAKVEKGKSKKLKSVKELLGI
TIMERSSFEKNPIDFLEAKGYKEVKKDLIIKLPKYSLFELENGRKRMLASARELQKGNELAL
PSKYVNFLYLASHYEKLKGSPEDNEQKQLFVEQHKHYLDEIIEQISEFSKRVILADANLDKV
LSAYNKHRDKPIREQAENIIHLFTLTNLGAPAAFKYFDTTIDRKQYRSTKEVLDATLIHQSI
TGLYETRIDLSQLGGDEGADKRTADGSEFESPKKKRKV

In some embodiments, the base editor further comprises a domain comprising all or a portion of a uracil glycosylase inhibitor (UGI). In some embodiments, the base editor comprises a domain comprising all or a portion of a uracil binding protein (UBP), such as a uracil DNA glycosylase (UDG). In some embodiments, the base editor comprises a domain comprising all or a portion of a nucleic acid polymerase. In some embodiments, a nucleic acid polymerase or portion thereof incorporated into a base editor is a translesion DNA polymerase.

In some embodiments, a domain of the base editor can comprise multiple domains. For example, the base editor comprising a polynucleotide programmable nucleotide binding domain derived from Cas9 can comprise an REC lobe and an NUC lobe corresponding to the REC lobe and NUC lobe of a wild-type or natural Cas9. In another example, the base editor can comprise one or more of a RuvCI domain, BH domain, REC1 domain, REC2 domain, RuvCII domain, L1 domain, HNH domain, L2 domain, RuvCIII domain, WED domain, TOPO domain or CTD domain. In some embodiments, one or more domains of the base editor comprise a mutation (e.g., substitution, insertion, deletion) relative to a wild type version of a polypeptide comprising the domain. For example, an HNH domain of a polynucleotide programmable DNA binding domain can comprise an H840A substitution. In another example, a RuvCI domain of a polynucleotide programmable DNA binding domain can comprise a D10A substitution.

Different domains (e.g., adjacent domains) of the base editor disclosed herein can be connected to each other with or without the use of one or more linker domains (e.g., an XTEN linker domain). In some embodiments, a linker domain can be a bond (e.g., covalent bond), chemical group, or a molecule linking two molecules or moieties, e.g., two domains of a fusion protein, such as, for example, a first domain (e.g., Cas9-derived domain) and a second domain (e.g., an adenosine deaminase domain or a cytidine deaminase domain). In some embodiments, a linker is a covalent bond (e.g., a carbon-carbon bond, disulfide bond, carbon-hetero atom bond, etc.). In certain embodiments, a linker is a carbon nitrogen bond of an amide linkage. In certain embodiments, a linker is a cyclic or acyclic, substituted or unsubstituted, branched or unbranched aliphatic or heteroaliphatic linker. In certain embodiments, a linker is polymeric (e.g., polyethylene, polyethylene glycol, polyamide, polyester, etc.). In certain embodiments, a linker comprises a monomer, dimer, or polymer of aminoalkanoic acid. In some embodiments, a linker comprises an aminoalkanoic acid (e.g., glycine, ethanoic acid, alanine, beta-alanine, 3-aminopropanoic acid, 4-aminobutanoic acid, 5-pentanoic acid, etc.). In some embodiments, a linker comprises a monomer, dimer, or polymer of aminohexanoic acid (Ahx). In certain embodiments, a linker is based on a carbocyclic moiety (e.g., cyclopentane, cyclohexane). In other embodiments, a linker comprises a polyethylene glycol moiety (PEG). In certain embodiments, a linker comprises an aryl or heteroaryl moiety. In certain embodiments, the linker is based on a phenyl ring. A linker can include functionalized moieties to facilitate attachment of a nucleophile (e.g., thiol, amino) from the peptide to the linker. Any electrophile can be used as part of the linker. Exemplary electrophiles include, but are not limited to, activated esters, activated amides, Michael acceptors, alkyl halides, aryl halides, acyl halides, and isothiocyanates. In some embodiments, a linker joins a gRNA binding domain of an RNA-programmable nuclease, including a Cas9 nuclease domain, and the catalytic domain of a nucleic acid editing protein. In some embodiments, a linker joins a dCas9 and a second domain (e.g., UGI, cytidine deaminase, etc.).

Typically, a linker is positioned between, or flanked by, two groups, molecules, or other moieties and connected to each one via a covalent bond, thus connecting the two. In some embodiments, a linker is an amino acid or a plurality of amino acids (e.g., a peptide or protein). In some embodiments, a linker is an organic molecule, group, polymer, or chemical moiety. In some embodiments, a linker is 2-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length. Longer or shorter linkers are also contemplated. In some embodiments, a linker domain comprises the amino acid sequence SGSETPGTSESATPES, which can also be referred to as the XTEN linker. Any method for linking the fusion protein domains can be employed (e.g., ranging from very flexible linkers of the form (SGGS)n, (GGGS)n, (GGGGS)n, and (G)n, to more rigid linkers of the form (EAAAK)n, (GGS)n, SGSETPGTSESATPES (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference), or (XP)n motif, in order to achieve the optimal length for activity for the nucleobase editor. In some embodiments, n is 1, 2, 3, 4, 5, 6, 7, 8, 9, 10, 11, 12, 13, 14, or 15. In some embodiments, the linker comprises a (GGS)n motif, wherein n is 1, 3, or 7. In some embodiments, the Cas9 domain of the fusion proteins provided herein are fused via a linker comprising the amino acid sequence SGSETPGTSESATPES. In some embodiments, a linker comprises a plurality of proline residues and is 5-21, 5-14, 5-9, 5-7 amino acids in length, e.g., PAPAP, PAPAPA, PAPAPAP, PAPAPAPA, P(AP)4, P(AP)7, P(AP)10 (see, e.g., Tan J, Zhang F, Karcher D, Bock R. Engineering of high-precision base editors for site-specific single nucleotide replacement. Nat Commun. 2019 Jan. 25; 10(1):439; the entire contents are incorporated herein by reference). Such proline-rich linkers are also termed “rigid” linkers.

### Linkers

In certain embodiments, linkers may be used to link any of the peptides or peptide domains of the invention. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond (e.g., a covalent bond), an organic molecule, group, polymer, or chemical moiety. In some embodiments, the linker is about 3 to about 104 (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 55, 60, 65, 70, 75, 80, 85, 90, 95, or 100) amino acids in length.

In some embodiments, the cytidine deaminase and adenosine deaminase and the napDNAbp are fused via a linker that is 4, 16, 32, or 104 amino acids in length. In some embodiments, the linker is about 3 to about 104 amino acids in length. In some embodiments, any of the fusion proteins provided herein, comprise a cytidine deaminase, adenosine deaminase and a Cas9 domain that are fused to each other via a linker. Various linker lengths and flexibilities between the deaminase domain (e.g., an engineered ecTadA) and the Cas9 domain can be employed (e.g., ranging from very flexible linkers of the form (GGGS)ₙ, (GGGGS)ₙ, and (G)ₙ to more rigid linkers of the form (EAAAK)ₙ, (SGGS)ₙ, SGSETPGTSESATPES (see, e.g., Guilinger J P, Thompson D B, Liu D R. Fusion of catalytically inactive Cas9 to FokI nuclease improves the specificity of genome modification. Nat. Biotechnol. 2014; 32(6): 577-82; the entire contents are incorporated herein by reference) and (XP)ₙ) in order to achieve the optimal length for activity for the nucleobase editor. In some embodiments, n is 1, 2, 3, 4, 5, 6, 7, 8, 9, 10, 11, 12, 13, 14, or 15. In some embodiments, the linker comprises a (GGS)ₙ motif, wherein n is 1, 3, or 7. In some embodiments, the cytidine deaminase and adenosine deaminase and the Cas9 domain of any of the fusion proteins provided herein are fused via a linker (e.g., an XTEN linker) comprising the amino acid sequence SGSETPGTSESATPES.

In some embodiments, the target region comprises a target window, wherein the target window comprises the target nucleobase pair. In some embodiments, the target window comprises 1-10 nucleotides. In some embodiments, the target window is 1, 2, 3, 4, 5, 6, 7, 8, 9, 10, 11, 12, 13, 14, 15, 16, 17, 18, 19, or 20 nucleotides in length. In some embodiments, the intended edit of base pair is within the target window. In some embodiments, the target window comprises the intended edit of base pair. In some embodiments, the method is performed using any of the base editors provided herein. In some embodiments, a target window is a deamination window.

Additionally, in some cases, a Gam protein can be fused to an N terminus of a base editor. In some cases, a Gam protein can be fused to a C-terminus of a base editor. The Gam protein of bacteriophage Mu can bind to the ends of double strand breaks (DSBs) and protect them from degradation. In some embodiments, using Gam to bind the free ends of DSB can reduce indel formation during the process of base editing. In some embodiments, 174-residue Gam protein is fused to the N terminus of the base editors. See. Komor, A. C., et al., “Improved base excision repair inhibition and bacteriophage Mu Gam protein yields C:G-to-T:A base editors with higher efficiency and product purity” Science Advances 3:eaao4774 (2017). In some cases, a mutation or mutations can change the length of a base editor domain relative to a wild type domain. For example, a deletion of at least one amino acid in at least one domain can reduce the length of the base editor. In another case, a mutation or mutations do not change the length of a domain relative to a wild type domain. For example, substitution(s) in any domain does/do not change the length of the base editor.

In some embodiments, the base editing fusion proteins provided herein need to be positioned at a precise location, for example, where a target base is placed within a defined region (e.g., a “deamination window”). In some cases, a target can be within a 4 base region. In some cases, such a defined target region can be approximately 15 bases upstream of the PAM. See Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

A defined target region can be a deamination window. A deamination window can be the defined region in which a base editor acts upon and deaminates a target nucleotide. In some embodiments, the deamination window is within a 2, 3, 4, 5, 6, 7, 8, 9, or 10 base regions. In some embodiments, the deamination window is 5, 6, 7, 8, 9, 10, 11, 12, 13, 14, 15, 16, 17, 18, 19, 20, 21, 22, 23, 24, or 25 bases upstream of the PAM.

The base editors of the present disclosure can comprise any domain, feature or amino acid sequence which facilitates the editing of a target polynucleotide sequence. For example, in some embodiments, the base editor comprises a nuclear localization sequence (NLS). In some embodiments, an NLS of the base editor is localized between a deaminase domain and a polynucleotide programmable nucleotide binding domain. In some embodiments, an NLS of the base editor is localized C-terminal to a polynucleotide programmable nucleotide binding domain.

Other exemplary features that can be present in a base editor as disclosed herein are localization sequences, such as cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

Non-limiting examples of protein domains which can be included in the fusion protein include deaminase domains (e.g., cytidine deaminase, adenosine deaminase), a uracil glycosylase inhibitor (UGI) domain, epitope tags, and reporter gene sequences.

Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). Additional protein sequences can include amino acid sequences that bind DNA molecules or bind other cellular molecules, including, but not limited to, maltose binding protein (MBP), S-tag, Lex A DNA binding domain (DBD) fusions, GAL4 DNA binding domain fusions, and herpes simplex virus (HSV) BP16 protein fusions.

### Base Editor Efficiency

CRISPR-Cas9 nucleases have been widely used to mediate targeted genome editing. In most genome editing applications, Cas9 forms a complex with a guide polynucleotide (e.g., single guide RNA (sgRNA)) and induces a double-stranded DNA break (DSB) at the target site specified by the sgRNA sequence. Cells primarily respond to this DSB through the non-homologous end-joining (NHEJ) repair pathway, which results in stochastic insertions or deletions (indels) that can cause frameshift mutations that disrupt the gene. In the presence of a donor DNA template with a high degree of homology to the sequences flanking the DSB, gene alteration can be achieved through an alternative pathway known as homology directed repair (HDR). Unfortunately, under most non-perturbative conditions, HDR is inefficient, dependent on cell state and cell type, and dominated by a larger frequency of indels. Base editing systems as provided herein provide a new way to provide genome editing without generating double-strand DNA breaks, without requiring a donor DNA template, and without inducing an excess of stochastic insertions and deletions.

The base editors provided herein are capable of modifying a specific nucleotide base without generating a significant proportion of indels. The term “indel(s)”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the target nucleotide sequence. In certain embodiments, any of the base editors provided herein are capable of generating a greater proportion of intended modifications (e.g., point mutations or deaminations) versus indels. In some embodiments, any of base editor systems provided herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, any of base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% indel formation in the target polynucleotide sequence.

In some embodiments, any of base editor systems comprising one of the ABE8 base editor variants described herein result in less than 0.8% indel formation in the target polynucleotide sequence. In some embodiments, any of base editor systems comprising one of the ABE8 base editor variants described herein result in at most 0.8% indel formation in the target polynucleotide sequence. In some embodiments, any of base editor systems comprising one of the ABE8 base editor variants described herein result in less than 0.3% indel formation in the target polynucleotide sequence. In some embodiments, any of base editor systems comprising one of the ABE8 base editor variants described results in lower indel formation in the target polynucleotide sequence compared to a base editor system comprising one of ABE7 base editors. In some embodiments, any of base editor systems comprising one of the ABE8 base editor variants described herein results in lower indel formation in the target polynucleotide sequence compared to a base editor system comprising an ABE7.10.

In some embodiments, any of base editor systems comprising one of the ABE8 base editor variants described herein has reduction in indel frequency compared to a base editor system comprising one of the ABE7 base editors. In some embodiments, any of base editor systems comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising one of the ABE7 base editors. In some embodiments, a base editor system comprising one of the ABE8 base editor variants described herein has at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% reduction in indel frequency compared to a base editor system comprising an ABE7.10.

The disclosure provides adenosine deaminase variants (e.g., ABE8 variants) that have increased efficiency and specificity. In particular, the adenosine deaminase variants described herein are more likely to edit a desired base within a polynucleotide, and are less likely to edit bases that are not intended to be altered (e.g., “bystanders”).

In some embodiments, any of the base editing system comprising one of the ABE8 base editor variants described herein has reduced bystander editing or mutations. In some embodiments, an unintended editing or mutation is a bystander mutation or bystander editing, for example, base editing of a target base (e.g., A or C) in an unintended or non-target position in a target window of a target nucleotide sequence. In some embodiments, any of the base editing system comprising one of the ABE8 base editor variants described herein has reduced bystander editing or mutations compared to a base editor system comprising an ABE7 base editor, e.g., ABE7.10. In some embodiments, any of the base editing system comprising one of the ABE8 base editor variants described herein has reduced bystander editing or mutations by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced bystander editing or mutations by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

In some embodiments, any of the base editing system comprising one of the ABE8 base editor variants described herein has reduced spurious editing. In some embodiments, an unintended editing or mutation is a spurious mutation or spurious editing, for example, non-specific editing or guide independent editing of a target base (e.g., A or C) in an unintended or non-target region of the genome. In some embodiments, any of the base editing system comprising one of the ABE8 base editor variants described herein has reduced spurious editing compared to a base editor system comprising an ABE7 base editor, e.g., ABE7.10. In some embodiments, any of the base editing system comprising one of the ABE8 base editor variants described herein has reduced spurious editing by at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% compared to a base editor system comprising an ABE7 base editor, e.g., ABE7.10. In some embodiments, any of the base editing system comprising one of the ABE8 base editor variants described herein has reduced spurious editing by at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold compared to a base editor system comprising an ABE7 base editor, e.g., ABE7.10.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations (i.e., mutation of bystanders). In some embodiments, any of the base editors provided herein are capable of generating at least 0.01% of intended mutations (i.e. at least 0.01% base editing efficiency). In some embodiments, any of the base editors provided herein are capable of generating at least 0.01%, 1, 2%, 3%, 4%, 5%10%, 15%, 20%, 25%, 30%, 40%, 45%, 50%, 60%, 70%, 80%, 90%, 95%, or 99% of intended mutations.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% base editing efficiency. In some embodiments, the base editing efficiency may be measured by calculating the percentage of edited nucleobases in a population of cells. In some embodiments, any of the ABE8 base editor variants described herein have base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases in a population of cells.

In some embodiments, any of the ABE8 base editor variants described herein has higher base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher base editing efficiency compared to an ABE7 base editor, e.g., ABE7.10.

In some embodiments, any of the ABE8 base editor variants described herein have at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% on-target base editing efficiency. In some embodiments, any of the ABE8 base editor variants described herein have on-target base editing efficiency of at least 0.01%, at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited target nucleobases in a population of cells.

In some embodiments, any of the ABE8 base editor variants described herein has higher on-target base editing efficiency compared to the ABE7 base editors. In some embodiments, any of the ABE8 base editor variants described herein have at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 450%, or at least 500% higher on-target base editing efficiency compared to an ABE7 base editor, e.g., ABE7.10.

In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target base editing efficiency compared to an ABE7 base editor, e.g., ABE7.10.

The ABE8 base editor variants described herein may be delivered to a host cell via a plasmid, a vector, a LNP complex, or an mRNA. In some embodiments, any of the ABE8 base editor variants described herein is delivered to a host cell as an mRNA. In some embodiments, an ABE8 base editor delivered via a nucleic acid based delivery system, e.g., an mRNA, has on-target editing efficiency of at least at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% as measured by edited nucleobases. In some embodiments, an ABE8 base editor delivered by an mRNA system has higher base editing efficiency compared to an ABE8 base editor delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, at least 99%, at least 100%, at least 105%, at least 110%, at least 115%, at least 120%, at least 125%, at least 130%, at least 135%, at least 140%, at least 145%, at least 150%, at least 155%, at least 160%, at least 165%, at least 170%, at least 175%, at least 180%, at least 185%, at least 190%, at least 195%, at least 200%, at least 210%, at least 220%, at least 230%, at least 240%, at least 250%, at least 260%, at least 270%, at least 280%, at least 290%, at least 300% higher, at least 310%, at least 320%, at least 330%, at least 340%, at least 350%, at least 360%, at least 370%, at least 380%, at least 390%, at least 400%, at least 450%, or at least 500% on-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 3.1 fold, at least 3.2 fold, at least 3.3 fold, at least 3.4 fold, at least 3.5 fold, at least 3.6 fold, at least 3.7 fold, at least 3.8 fold, at least 3.9 fold, at least 4.0 fold, at least 4.1 fold, at least 4.2 fold, at least 4.3 fold, at least 4.4 fold, at least 4.5 fold, at least 4.6 fold, at least 4.7 fold, at least 4.8 fold, at least 4.9 fold, or at least 5.0 fold higher on-target editing efficiency when delivered by an mRNA system compared to when delivered by a plasmid or vector system.

In some embodiments, any of base editor systems comprising one of the ABE8 base editor variants described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in the target polynucleotide sequence.

In some embodiments, any of the ABE8 base editor variants described herein has lower guided off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guided off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, or at least 3.0 fold lower guided off-target editing efficiency when delivered by an mRNA system compared to when delivered by a plasmid or vector system. In some embodiments, any of the ABE8 base editor variants described herein has at least about 2.2 fold decrease in guided off-target editing efficiency when delivered by an mRNA system compared to when delivered by a plasmid or vector system.

In some embodiments, any of the ABE8 base editor variants described herein has lower guide-independent off-target editing efficiency when delivered by an mRNA system compared to when delivered by a plasmid or vector system. In some embodiments, any of the ABE8 base editor variants described herein has at least 1%, at least 2%, at least 3%, at least 4%, at least 5%, at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95%, or at least 99% lower guide-independent off-target editing efficiency when delivered by an mRNA system compared to when delivered by a plasmid or vector system. In some embodiments, any of the ABE8 base editor variants described herein has at least 1.1 fold, at least 1.2 fold, at least 1.3 fold, at least 1.4 fold, at least 1.5 fold, at least 1.6 fold, at least 1.7 fold, at least 1.8 fold, at least 1.9 fold, at least 2.0 fold, at least 2.1 fold, at least 2.2 fold, at least 2.3 fold, at least 2.4 fold, at least 2.5 fold, at least 2.6 fold, at least 2.7 fold, at least 2.8 fold, at least 2.9 fold, at least 3.0 fold, at least 5.0 fold, at least 10.0 fold, at least 20.0 fold, at least 50.0 fold, at least 70.0 fold, at least 100.0 fold, at least 120.0 fold, at least 130.0 fold, or at least 150.0 fold lower guide-independent off-target editing efficiency when delivered by an mRNA system compared to when delivered by a plasmid or vector system. In some embodiments, ABE8 base editor variants described herein has 134.0 fold decrease in guide-independent off-target editing efficiency (e.g., spurious RNA deamination) when delivered by an mRNA system compared to when delivered by a plasmid or vector system. In some embodiments, ABE8 base editor variants described herein does not increase guide-independent mutation rates across the genome.

Some aspects of the disclosure are based on the recognition that any of the base editors provided herein are capable of efficiently generating an intended mutation, such as a point mutation, in a nucleic acid (e.g., a nucleic acid within a genome of a subject) without generating a significant number of unintended mutations, such as unintended point mutations. In some embodiments, any of the base editors provided herein are capable of generating at least 0.01% of intended mutations (e.g., spurious off-target editing or bystander editing). In some embodiments, an intended mutation is a mutation that is generated by a specific base editor bound to a gRNA, specifically designed to alter or correct a mutation in a target gene. Some aspects of the disclosure are based on the recognition that any of the base editors provided herein are capable of efficiently generating an intended mutation in a nucleic acid (e.g. a nucleic acid within a genome of a subject) without generating a significant number of unintended mutations. In some embodiments, an intended mutation is a mutation that is generated by a specific base editor bound to a gRNA, specifically designed to alter or correct an intended mutation. In some embodiments, the intended mutation is a mutation that generates a stop codon, for example, a premature stop codon within the coding region of a gene. In some embodiments, the intended mutation is a mutation that eliminates a stop codon. In some embodiments, the intended mutation is a mutation that alters the splicing of a gene. In some embodiments, the intended mutation is a mutation that alters the regulatory sequence of a gene (e.g., a gene promotor or gene repressor). In some embodiments, the base editors provided herein are capable of generating a ratio of intended point mutations to indels that is greater than 1:1. In some embodiments, the base editors provided herein are capable of generating a ratio of intended point mutations to indels that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 8.5:1, at least 9:1, at least 10:1, at least 11:1, at least 12:1, at least 13:1, at least 14:1, at least 15:1, at least 20:1, at least 25:1, at least 30:1, at least 40:1, at least 50:1, at least 100:1, at least 200:1, at least 300:1, at least 400:1, at least 500:1, at least 600:1, at least 700:1, at least 800:1, at least 900:1, or at least 1000:1, or more.

The number of intended mutations and indels can be determined using any suitable method, for example, as described in International PCT Application Nos. PCT/2017/045381 (WO2018/027078) and PCT/US2016/058344 (WO2017/070632); Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017); the entire contents of which are hereby incorporated by reference.

In some embodiments, to calculate indel frequencies, sequencing reads are scanned for exact matches to two 10-bp sequences that flank both sides of a window in which indels can occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively. In some embodiments, the base editors provided herein can limit formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor.

The number of indels formed at a target nucleotide region can depend on the amount of time a nucleic acid (e.g., a nucleic acid within the genome of a cell) is exposed to a base editor. In some embodiments, the number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing the target nucleotide sequence (e.g., a nucleic acid within the genome of a cell) to a base editor. It should be appreciated that the characteristics of the base editors as described herein can be applied to any of the fusion proteins, or methods of using the fusion proteins provided herein.

In some embodiments, the base editors provided herein are capable of limiting formation of indels in a region of a nucleic acid. In some embodiments, the region is at a nucleotide targeted by a base editor or a region within 2, 3, 4, 5, 6, 7, 8, 9, or 10 nucleotides of a nucleotide targeted by a base editor. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, any number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

### Multiplex Editing

In some embodiments, the base editor system provided herein is capable of multiplex editing of a plurality of nucleobase pairs in one or more genes. In some embodiments, the plurality of nucleobase pairs is located in the same gene. In some embodiments, the plurality of nucleobase pairs is located in one or more gene, wherein at least one gene is located in a different locus. In some embodiments, the multiplex editing can comprise one or more guide polynucleotides. In some embodiments, the multiplex editing can comprise one or more base editor system. In some embodiments, the multiplex editing can comprise one or more base editor systems with a single guide polynucleotide. In some embodiments, the multiplex editing can comprise one or more base editor system with a plurality of guide polynucleotides. In some embodiments, the multiplex editing can comprise one or more guide polynucleotide with a single base editor system. In some embodiments, the multiplex editing can comprise at least one guide polynucleotide that does not require a PAM sequence to target binding to a target polynucleotide sequence. In some embodiments, the multiplex editing can comprise at least one guide polynucleotide that requires a PAM sequence to target binding to a target polynucleotide sequence. In some embodiments, the multiplex editing can comprise a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any of combination of the methods of using any of the base editor provided herein. It should also be appreciated that the multiplex editing using any of the base editors as described herein can comprise a sequential editing of a plurality of nucleobase pairs.

In some embodiments, the plurality of nucleobase pairs are in one more genes. In some embodiments, the plurality of nucleobase pairs is in the same gene. In some embodiments, at least one gene in the one more genes is located in a different locus.

In some embodiments, the editing is editing of the plurality of nucleobase pairs in at least one protein coding region. In some embodiments, the editing is editing of the plurality of nucleobase pairs in at least one protein non-coding region. In some embodiments, the editing is editing of the plurality of nucleobase pairs in at least one protein coding region and at least one protein non-coding region.

In some embodiments, the editing is in conjunction with one or more guide polynucleotides. In some embodiments, the base editor system can comprise one or more base editor system. In some embodiments, the base editor system can comprise one or more base editor systems in conjunction with a single guide polynucleotide. In some embodiments, the base editor system can comprise one or more base editor system in conjunction with a plurality of guide polynucleotides. In some embodiments, the editing is in conjunction with one or more guide polynucleotide with a single base editor system. In some embodiments, the editing is in conjunction with at least one guide polynucleotide that does not require a PAM sequence to target binding to a target polynucleotide sequence. In some embodiments, the editing is in conjunction with at least one guide polynucleotide that require a PAM sequence to target binding to a target polynucleotide sequence. In some embodiments, the editing is in conjunction with a mix of at least one guide polynucleotide that does not require a PAM sequence to target binding to a target polynucleotide sequence and at least one guide polynucleotide that require a PAM sequence to target binding to a target polynucleotide sequence. It should be appreciated that the characteristics of the multiplex editing using any of the base editors as described herein can be applied to any of combination of the methods of using any of the base editors provided herein. It should also be appreciated that the editing can comprise a sequential editing of a plurality of nucleobase pairs.

### Methods of Using Base Editors

Editing the HBV genome up new strategies for therapeutics and basic research.

The present disclosure provides methods for the treatment of a subject diagnosed with HBV infection. For example, in some embodiments, a method is provided that comprises administering to a subject having such a disease, e.g., a disease caused by HBV infection, an effective amount of a nucleobase editor (e.g., an adenosine deaminase base editor or a cytidine deaminase base editor) that alters a nucleobase in the HBV genome.

In a certain aspect, methods are provided for the treatment of HBV infection, which can be treated by deaminase mediated gene editing of at least one of the HBV genes.

It will be understood that the numbering of the specific positions or residues in the respective sequences, e.g., polynucleotide or amino acid sequences of a disease-related gene or its encoded protein, respectively, depends on the particular protein and numbering scheme used. Numbering can be different, e.g., in precursors of a mature protein and the mature protein itself, and differences in sequences from species to species can affect numbering. One of skill in the art will be able to identify the respective residue in any homologous protein and in the respective encoding nucleic acid by methods well known in the art, e.g., by sequence alignment and determination of homologous residues.

Provided herein are methods of using the base editor or base editor system for editing a nucleobase in a target nucleotide sequence in the HBV genome. In some embodiments, the activity of the base editor (e.g., comprising an adenosine deaminase and a Cas9 domain) results in an alteration of a nucleobase in the HBV genome. In some embodiments, the target DNA sequence is altered to comprise a G→A point mutation, and wherein the deamination of the A base results in a sequence that reduces or eliminates HBV function. In some embodiments, the target DNA sequence is altered to comprise a T→C point mutation, and wherein the deamination of the mutant C base results in a sequence that reduces or eliminates HBV function.

In some embodiments, the target DNA sequence encodes a protein (e.g., an HBV protein such as a polymerase or a surface antigen), and the alteration results in a change in the amino acid encoded by the wildtype codon. In some embodiments, the deamination of an A nucleotide results in a change of the amino acid encoded by the wildtype codon. In some embodiments, the deamination of the wildtype A results in the codon encoding a mutant amino acid. In some embodiments, the deamination of the wildtype C results in a change of the amino acid encoded by the wildtype codon. In some embodiments, the deamination of the wildtype C results in the codon encoding a mutant amino acid. In some embodiments, the subject has or has been diagnosed with HBV infection.

In some embodiments, the adenosine deaminases provided herein are capable of deaminating adenine of a deoxyadenosine residue of DNA. Other aspects of the disclosure provide fusion proteins that comprise an adenosine deaminase (e.g., an adenosine deaminase that deaminates deoxyadenosine in DNA as described herein) and a domain (e.g., a Cas9 or a Cpf1 protein) capable of binding to a specific nucleotide sequence. For example, the adenosine can be converted to an inosine residue, which typically base pairs with a cytosine residue. Such fusion proteins are useful inter alia for targeted editing of nucleic acid sequences. Such fusion proteins can be used for targeted editing of HBV DNA in vitro; for the introduction of targeted mutations, e.g., for the alteration of the HBV genome in cells ex vivo; and for the introduction of targeted mutations in vivo, e.g., the alteration of the HBV genome in cells in a subject infected with HBV. The present disclosure provides deaminases, fusion proteins, nucleic acids, vectors, cells, compositions, methods, kits, systems, etc. that utilize the deaminases and nucleobase editors.

### Generating an Intended Mutation

The nucleobase editing proteins provided herein can be validated for gene editing-based human therapeutics in vitro, e.g., by introducing alterations in the HBV genome that reduce or eliminate the infection or symptoms thereof. It will be understood by the skilled artisan that the nucleobase editing proteins provided herein, e.g., the fusion proteins comprising a polynucleotide programmable nucleotide binding domain (e.g., Cas9) and a nucleobase editing domain (e.g., an adenosine deaminase domain or a cytidine deaminase domain) can be used to alter any A to G or C to T. In the first case, deamination of the A to I alters the HBV genome sequence, and in the latter case, deamination of the A that is base-paired with a T in the HBV genome, followed by a round of replication, introduces the mutation.

In some embodiments, the present disclosure provides base editors that can efficiently generating an intended mutation in a nucleic acid (e.g., a nucleic acid within a genome of a subject) without generating a significant number of unintended mutations, such as unintended point mutations in a subject's genome. In some embodiments, an intended mutation is a mutation that is generated by a specific base editor (e.g., cytidine base editor or adenosine base editor) bound to a guide polynucleotide (e.g., gRNA), specifically designed to generate the intended mutation. In some embodiments, the intended mutation is an adenine (A) to guanine (G) point mutation. In some embodiments, the intended mutation is a cytosine (C) to thymine (T) point mutation. In some embodiments, the intended mutation is an adenine (A) to guanine (G) point mutation within the coding region or non-coding region of a gene. In some embodiments, the intended mutation is a cytosine (C) to thymine (T) point mutation within the coding region or non-coding region of a gene. In some embodiments, the intended mutation is a point mutation that generates a stop codon, for example, a premature stop codon within the coding region of a gene. In some embodiments, the intended mutation is a mutation that eliminates a stop codon.

In some embodiments, any of the base editors provided herein are capable of generating a ratio of intended mutations to unintended mutations (e.g., intended point mutations:unintended point mutations) that is greater than 1:1. In some embodiments, any of the base editors provided herein are capable of generating a ratio of intended mutations to unintended mutations (e.g., intended point mutations:unintended point mutations) that is at least 1.5:1, at least 2:1, at least 2.5:1, at least 3:1, at least 3.5:1, at least 4:1, at least 4.5:1, at least 5:1, at least 5.5:1, at least 6:1, at least 6.5:1, at least 7:1, at least 7.5:1, at least 8:1, at least 10:1, at least 12:1, at least 15:1, at least 20:1, at least 25:1, at least 30:1, at least 40:1, at least 50:1, at least 100:1, at least 150:1, at least 200:1, at least 250:1, at least 500:1, or at least 1000:1, or more

Details of base editor efficiency are described in International PCT Application Nos. PCT/2017/045381 (WO2018/027078) and PCT/US2016/058344 (WO2017/070632), each of which is incorporated herein by reference for its entirety. Also see Komor, A. C., et al., “Programmable editing of a target base in genomic DNA without double-stranded DNA cleavage” Nature 533, 420-424 (2016); Gaudelli, N. M., et al., “Programmable base editing of A·T to G·C in genomic DNA without DNA cleavage” Nature 551, 464-471 (2017); and Komor, A. C., et al., “Improved base excision repair inhibition and bacteriophage Mu Gam protein yields C:G-to-T:A base editors with higher efficiency and product purity” Science Advances 3:eaao4774 (2017), the entire contents of which are hereby incorporated by reference.

In some embodiments, the editing of the plurality of nucleobase pairs in one or more genes result in formation of at least one intended mutation. In some embodiments, the formation of the at least one intended mutation results in an alteration of the HBV genome. It should be appreciated that the characteristics of the multiplex editing of the base editors as described herein can be applied to any of combination of the methods of using the base editor provided herein.

### Modification of HBV Genes

In some embodiments, a precise modification of an HBV gene decreases the virulence of the virus and/or decreases the ability of the virus to propagate in vitro. The modification can be a premature stop codon or other mutation that impairs or otherwise reduces the activity of an HBV protein. In some embodiments, the HBV gene that is targeted for modification is the pol, X, S, pre-S1, pre-S2, or the core of pre-core genes, or a combination thereof.

### Expression of Fusion Proteins in a Host Cell

Fusion proteins of the disclosure comprising an adenosine deaminase variant may be expressed in virtually any host cell of interest, including but not limited to bacteria, yeast, fungi, insects, plants, and animal cells using routine methods known to the skilled artisan. For example, a DNA encoding an adenosine deaminase of the disclosure can be cloned by designing suitable primers for the upstream and downstream of CDS based on the cDNA sequence. The cloned DNA may be directly, or after digestion with a restriction enzyme when desired, or after addition of a suitable linker and/or a nuclear localization signal ligated with a DNA encoding one or more additional components of a base editing system. The base editing system is translated in a host cell to form a complex.

A DNA encoding a protein domain described herein can be obtained by chemically synthesizing the DNA, or by connecting synthesized partly overlapping oligoDNA short chains by utilizing the PCR method and the Gibson Assembly method to construct a DNA encoding the full length thereof. The advantage of constructing a full-length DNA by chemical synthesis or a combination of PCR method or Gibson Assembly method is that the codon to be used can be designed in CDS full-length according to the host into which the DNA is introduced. In the expression of a heterologous DNA, the protein expression level is expected to increase by converting the DNA sequence thereof to a codon highly frequently used in the host organism. As the data of codon use frequency in host to be used, for example, the genetic code use frequency database (http://www.kazusa.or.jp/codon/index.html) disclosed in the home page of Kazusa DNA Research Institute can be used, or documents showing the codon use frequency in each host may be referred to. By reference to the obtained data and the DNA sequence to be introduced, codons showing low use frequency in the host from among those used for the DNA sequence may be converted to a codon coding the same amino acid and showing high use frequency.

An expression vector containing a DNA encoding a nucleic acid sequence-recognizing module and/or a nucleic acid base converting enzyme can be produced, for example, by linking the DNA to the downstream of a promoter in a suitable expression vector.

When the expression vectors are Escherichia coli-derived plasmids (e.g., pBR322, pBR325, pUC12, pUC13); Bacillus subtilis-derived plasmids (e.g., pUB110, pTP5, pC194); yeast-derived plasmids (e.g., pSH19, pSH15); insect cell expression plasmids (e.g., pFast-Bac); animal cell expression plasmids (e.g., pA1-11, pXT1, pRc/CMV, pRc/RSV, pcDNAI/Neo); bacteriophages such as .lamda.phage and the like; insect virus vectors such as baculovirus and the like (e.g., BmNPV, AcNPV); animal virus vectors such as retrovirus, vaccinia virus, adenovirus and the like are used.

In some embodiments, any promoter appropriate for a host to be used for gene expression can be used. In a conventional method using DSB, since the survival rate of the host cell sometimes decreases markedly due to the toxicity, it is desirable to increase the number of cells by the start of the induction by using an inductive promoter. However, since sufficient cell proliferation can also be afforded by expressing the nucleic acid-modifying enzyme complex of the present disclosure, a constitution promoter can also be used without limitation.

For example, when the host is an animal cell, SRα promoter, SV40 promoter, LTR promoter, CMV (cytomegalovirus) promoter, RSV (Rous sarcoma virus) promoter, MoMuLV (Moloney mouse leukemia virus) LTR, HSV-TK (simple herpes virus thymidine kinase) promoter and the like are used. Of these, CMV promoter, SRa promoter and the like are preferable.

When the host is Escherichia coli, trp promoter, lac promoter, recA promoter, λP_{L} promoter, lpp promoter, T7 promoter and the like are preferable.

When the host is genus Bacillus, SPO1 promoter, SPO2 promoter, penP promoter and the like are preferable.

When the host is a yeast, Gall/10 promoter, PHO5 promoter, PGK promoter, GAP promoter, ADH promoter and the like are preferable.

When the host is an insect cell, polyhedrin promoter, P10 promoter and the like are preferable.

When the host is a plant cell, CaMV35S promoter, CaMV19S promoter, NOS promoter and the like are preferable.

As the expression vector, besides those mentioned above, one containing enhancer, splicing signal, terminator, polyA addition signal, a selection marker such as drug resistance gene, auxotrophic complementary gene and the like, replication origin and the like on demand can be used.

An RNA encoding a protein domain described herein can be prepared by, for example, transcription to mRNA in a vitro transcription system known per se by using a vector encoding DNA encoding the above-mentioned nucleic acid sequence-recognizing module and/or a nucleic acid base converting enzyme as a template.

A fusion protein of the disclosure can be intracellularly expressed by introducing an expression vector containing a DNA encoding a nucleic acid sequence-recognizing module and/or a nucleic acid base converting enzyme into a host cell, and culturing the host cell.

As the host, genus Escherichia, genus Bacillus, yeast, insect cell, insect, animal cell and the like are used.

As the genus Escherichia, Escherichia coli K12.cndot.DH1 (Proc. Natl. Acad. Sci. USA, 60, 160 (1968)), Escherichia coli JM103 (Nucleic Acids Research, 9, 309 (1981)), Escherichia coli JA221 (Journal of Molecular Biology, 120, 517 (1978)), Escherichia coli HB101 (Journal of Molecular Biology, 41, 459 (1969)), Escherichia coli C600 (Genetics, 39, 440 (1954)) and the like are used.

As the genus Bacillus, Bacillus subtilis M1114 (Gene, 24, 255 (1983)), Bacillus subtilis 207-21 (Journal of Biochemistry, 95, 87 (1984)) and the like are used.

As the yeast, Saccharomyces cerevisiae AH22, AH22R, NA87-11A, DKD-5D, 20B-12, Schizosaccharomyces pombe NCYC1913, NCYC2036, Pichia pastoris KM71 and the like are used.

As the insect cell when the virus is AcNPV, cells of cabbage armyworm larva-derived established line (Spodoptera frugiperda cell; Sf cell), MG1 cells derived from the mid-intestine of Trichoplusia ni, High Five™ cells derived from an egg of Trichoplusia ni, Mamestra brassicae-derived cells, Estigmena acrea-derived cells and the like are used. When the virus is BmNPV, cells of Bombyx mori-derived established line (Bombyx mori N cell; BmN cell) and the like are used as insect cells. As the Sf cell, for example, Sf9 cell (ATCC CRL1711), Sf21 cell (all above, In Vivo, 13, 213-217 (1977)) and the like are used.

As the insect, for example, larva of Bombyx mori, Drosophila, cricket and the like are used (Nature, 315, 592 (1985)).

As the animal cell, cell lines such as monkey COS-7 cell, monkey Vero cell, Chinese hamster ovary (CHO) cell, dhfr gene-deficient CHO cell, mouse L cell, mouse AtT-20 cell, mouse myeloma cell, rat GH3 cell, human FL cell and the like, pluripotent stem cells such as iPS cell, ES cell and the like of human and other mammals, and primary cultured cells prepared from various tissues are used. Furthermore, zebrafish embryo, Xenopus oocyte and the like can also be used.

As the plant cell, suspend cultured cells, callus, protoplast, leaf segment, root segment and the like prepared from various plants (e.g., grain such as rice, wheat, corn and the like, product crops such as tomato, cucumber, eggplant and the like, garden plants such as carnation, Eustoma russellianum and the like, experiment plants such as tobacco, Arabidopsis thaliana and the like) are used.

All the above-mentioned host cells may be haploid (monoploid), or polyploid (e.g., diploid, triploid, tetraploid and the like). In the conventional mutation introduction methods, mutation is, in principle, introduced into only one homologous chromosome to produce a hetero gene type. Therefore, desired phenotype is not expressed unless dominant mutation occurs, and homozygousness inconveniently requires labor and time. In contrast, according to the present disclosure, since mutation can be introduced into any allele on the homologous chromosome in the genome, desired phenotype can be expressed in a single generation even in the case of recessive mutation, which is extremely useful since the problem of the conventional method can be solved.

An expression vector can be introduced by a known method (e.g., lysozyme method, competent method, PEG method, CaCl₂) coprecipitation method, electroporation method, the microinjection method, the particle gun method, lipofection method, Agrobacterium method and the like) according to the kind of the host.

Escherichia coli can be transformed according to the methods described in, for example, Proc. Natl. Acad. Sci. USA, 69, 2110 (1972), Gene, 17, 107 (1982) and the like.

The genus Bacillus can be introduced into a vector according to the methods described in, for example, Molecular & General Genetics, 168, 111 (1979) and the like.

A yeast can be introduced into a vector according to the methods described in, for example, Methods in Enzymology, 194, 182-187 (1991), Proc. Natl. Acad. Sci. USA, 75, 1929 (1978) and the like.

An insect cell and an insect can be introduced into a vector according to the methods described in, for example, Bio/Technology, 6, 47-55 (1988) and the like.

An animal cell can be introduced into a vector according to the methods described in, for example, Cell Engineering additional volume 8, New Cell Engineering Experiment Protocol, 263-267 (1995) (published by Shujunsha), and Virology, 52, 456 (1973).

A cell introduced with a vector can be cultured according to a known method according to the kind of the host.

For example, when Escherichia coli or genus Bacillus is cultured, a liquid medium is preferable as a medium to be used for the culture. The medium preferably contains a carbon source, nitrogen source, inorganic substance and the like necessary for the growth of the transformant. Examples of the carbon source include glucose, dextrin, soluble starch, sucrose and the like; examples of the nitrogen source include inorganic or organic substances such as ammonium salts, nitrate salts, corn steep liquor, peptone, casein, meat extract, soybean cake, potato extract and the like; and examples of the inorganic substance include calcium chloride, sodium dihydrogen phosphate, magnesium chloride and the like. The medium may contain yeast extract, vitamins, growth promoting factor and the like. The pH of the medium is preferably about 5 to about 8.

As a medium for culturing Escherichia coli, for example, M9 medium containing glucose, casamino acid (Journal of Experiments in Molecular Genetics, 431-433, Cold Spring Harbor Laboratory, New York 1972) is preferable. Where necessary, for example, agents such as 3β-indolylacrylic acid may be added to the medium to ensure an efficient function of a promoter. Escherichia coli is cultured at generally about 15 to about 43° C. Where necessary, aeration and stirring may be performed.

The genus Bacillus is cultured at generally about 30° C. to about 40° C. Where necessary, aeration and stirring may be performed.

Examples of the medium for culturing yeast include Burkholder minimum medium (Proc. Natl. Acad. Sci. USA, 77, 4505 (1980)), SD medium containing 0.5% casamino acid (Proc. Natl. Acad. Sci. USA, 81, 5330 (1984)) and the like. The pH of the medium is preferably about 5 to about 8. The culture is performed at generally about 20° C. to about 35° C. Where necessary, aeration and stirring may be performed.

As a medium for culturing an insect cell or insect, for example, Grace's Insect Medium (Nature, 195, 788 (1962)) containing an additive such as inactivated 10% bovine serum and the like as appropriate and the like are used. The pH of the medium is preferably about 6.2 to about 6.4. The culture is performed at generally about 27° C. Where necessary, aeration and stirring may be performed.

As a medium for culturing an animal cell, for example, minimum essential medium (MEM) containing about 5 to about 20% of fetal bovine serum (Science, 122, 501 (1952)), Dulbecco's modified Eagle medium (DMEM) (Virology, 8, 396 (1959)), RPMI 1640 medium (The Journal of the American Medical Association, 199, 519 (1967)), 199 medium (Proceeding of the Society for the Biological Medicine, 73, 1 (1950)) and the like are used. The pH of the medium is preferably about 6 to about 8. The culture is performed at generally about 30° C. to about 40° C. Where necessary, aeration and stirring may be performed.

As a medium for culturing a plant cell, for example, MS medium, LS medium, B5 medium and the like are used. The pH of the medium is preferably about 5 to about 8. The culture is performed at generally about 20° C. to about 30° C. Where necessary, aeration and stirring may be performed.

When a higher eukaryotic cell, such as animal cell, insect cell, plant cell and the like is used as a host cell, a DNA encoding a base editing system of the present disclosure (e.g., comprising an adenosine deaminase variant) is introduced into a host cell under the regulation of an inducible promoter (e.g., metallothionein promoter (induced by heavy metal ion), heat shock protein promoter (induced by heat shock), Tet-ON/Tet-OFF system promoter (induced by addition or removal of tetracycline or a derivative thereof), steroid-responsive promoter (induced by steroid hormone or a derivative thereof) etc.), the induction substance is added to the medium (or removed from the medium) at an appropriate stage to induce expression of the nucleic acid-modifying enzyme complex, culture is performed for a given period to carry out a base editing and, introduction of a mutation into a target gene, transient expression of the base editing system can be realized.

Prokaryotic cells such as Escherichia coli and the like can utilize an inducible promoter. Examples of the inducible promoter include, but are not limited to, lac promoter (induced by IPTG), cspA promoter (induced by cold shock), araBAD promoter (induced by arabinose) and the like.

Alternatively, the above-mentioned inductive promoter can also be utilized as a vector removal mechanism when higher eukaryotic cells, such as animal cell, insect cell, plant cell and the like are used as a host cell. That is, a vector is mounted with a replication origin that functions in a host cell, and a nucleic acid encoding a protein necessary for replication (e.g., SV40 on and large T antigen, oriP and EBNA-1 etc. for animal cells), of the expression of the nucleic acid encoding the protein is regulated by the above-mentioned inducible promoter. As a result, while the vector is autonomously replicatable in the presence of an induction substance, when the induction substance is removed, autonomous replication is not available, and the vector naturally falls off along with cell division (autonomous replication is not possible by the addition of tetracycline and doxycycline in Tet-OFF system vector).

### Delivery System

A base editor disclosed herein can be encoded on a nucleic acid that is contained in a viral vector. Viral vectors can include lentivirus, Adenovirus, Retrovirus, and Adeno-associated viruses (AAVs). Viral vectors can be selected based on the application. For example, AAVs are commonly used for gene delivery in vivo due to their mild immunogenicity. Adenoviruses are commonly used as vaccines because of the strong immunogenic response they induce. Packaging capacity of the viral vectors can limit the size of the base editor that can be packaged into the vector. For example, the packaging capacity of the AAVs is ˜4.5 kb including two 145 base inverted terminal repeats (ITRs).

AAV is a small, single-stranded DNA dependent virus belonging to the parvovirus family. The 4.7 kb wild-type (wt) AAV genome is made up of two genes that encode four replication proteins and three capsid proteins, respectively, and is flanked on either side by 145-bp inverted terminal repeats (ITRs). The virion is composed of three capsid proteins, Vp1, Vp2, and Vp3, produced in a 1:1:10 ratio from the same open reading frame but from differential splicing (Vp1) and alternative translational start sites (Vp2 and Vp3, respectively). Vp3 is the most abundant subunit in the virion and participates in receptor recognition at the cell surface defining the tropism of the virus. A phospholipase domain, which functions in viral infectivity, has been identified in the unique N terminus of Vp1.

Similar to wt AAV, recombinant AAV (rAAV) utilizes the cis-acting 145-bp ITRs to flank vector transgene cassettes, providing up to 4.5 kb for packaging of foreign DNA. Subsequent to infection, rAAV can express a fusion protein of the invention and persist without integration into the host genome by existing episomally in circular head-to-tail concatemers. Although there are numerous examples of rAAV success using this system, in vitro and in vivo, the limited packaging capacity has limited the use of AAV-mediated gene delivery when the length of the coding sequence of the gene is equal or greater in size than the wt AAV genome.

The small packaging capacity of AAV vectors makes the delivery of a number of genes that exceed this size and/or the use of large physiological regulatory elements challenging. These challenges can be addressed, for example, by dividing the protein(s) to be delivered into two or more fragments, wherein the N-terminal fragment is fused to a split intein-N and the C-terminal fragment is fused to a split intein-C. These fragments are then packaged into two or more AAV vectors. As used herein, “intein” refers to a self-splicing protein intron (e.g., peptide) that ligates flanking N-terminal and C-terminal exteins (e.g., fragments to be joined). The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

A fragment of a fusion protein of the invention can vary in length. In some embodiments, a protein fragment ranges from 2 amino acids to about 1000 amino acids in length. In some embodiments, a protein fragment ranges from about 5 amino acids to about 500 amino acids in length. In some embodiments, a protein fragment ranges from about 20 amino acids to about 200 amino acids in length. In some embodiments, a protein fragment ranges from about 10 amino acids to about 100 amino acids in length. Suitable protein fragments of other lengths will be apparent to a person of skill in the art.

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In one embodiment, dual AAV vectors are generated by splitting a large transgene expression cassette in two separate halves (5′ and 3′ ends, or head and tail), where each half of the cassette is packaged in a single AAV vector (of <5 kb). The re-assembly of the full-length transgene expression cassette is then achieved upon co-infection of the same cell by both dual AAV vectors followed by: (1) homologous recombination (HR) between 5′ and 3′ genomes (dual AAV overlapping vectors); (2) ITR-mediated tail-to-head concatemerization of 5′ and 3′ genomes (dual AAV trans-splicing vectors); or (3) a combination of these two mechanisms (dual AAV hybrid vectors). The use of dual AAV vectors in vivo results in the expression of full-length proteins. The use of the dual AAV vector platform represents an efficient and viable gene transfer strategy for transgenes of >4.7 kb in size.

The disclosed strategies for designing base editors can be useful for generating base editors capable of being packaged into a viral vector. The use of RNA or DNA viral based systems for the delivery of a base editor takes advantage of highly evolved processes for targeting a virus to specific cells in culture or in the host and trafficking the viral payload to the nucleus or host cell genome. Viral vectors can be administered directly to cells in culture, patients (in vivo), or they can be used to treat cells in vitro, and the modified cells can optionally be administered to patients (ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (See, e.g., Buchscher et al., J. Virol. 66:2731-2739 (1992); Johann et al., J. Virol. 66:1635-1640 (1992); Sommnerfelt et al., Virol. 176:58-59 (1990); Wilson et al., J. Virol. 63:2374-2378 (1989); Miller et al., J. Virol. 65:2220-2224 (1991); PCT/US94/05700).

Retroviral vectors, especially lentiviral vectors, can require polynucleotide sequences smaller than a given length for efficient integration into a target cell. For example, retroviral vectors of length greater than 9 kb can result in low viral titers compared with those of smaller size. In some aspects, a base editor of the present disclosure is of sufficient size so as to enable efficient packaging and delivery into a target cell via a retroviral vector. In some cases, a base editor is of a size so as to allow efficient packing and delivery even when expressed together with a guide nucleic acid and/or other components of a targetable nuclease system.

In applications where transient expression is preferred, adenoviral based systems can be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors can also be used to transduce cells with target nucleic acids, e.g., in the in vitro production of nucleic acids and peptides, and for in vivo and ex vivo gene therapy procedures (See, e.g., West et al., Virology 160:38-47 (1987); U.S. Pat. No. 4,797,368; WO 93/24641; Kotin, Human Gene Therapy 5:793-801 (1994); Muzyczka, J. Clin. Invest. 94:1351 (1994). The construction of recombinant AAV vectors is described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., J. Virol. 63:03822-3828 (1989).

A base editor described herein can therefore be delivered with viral vectors. One or more components of the base editor system can be encoded on one or more viral vectors. For example, a base editor and guide nucleic acid can be encoded on a single viral vector. In other cases, the base editor and guide nucleic acid are encoded on different viral vectors. In either case, the base editor and guide nucleic acid can each be operably linked to a promoter and terminator.

The combination of components encoded on a viral vector can be determined by the cargo size constraints of the chosen viral vector.

### Non-Viral Delivery of Base Editors

Non-viral delivery approaches for base editors are also available. One important category of non-viral nucleic acid vectors are nanoparticles, which can be organic or inorganic. Nanoparticles are well known in the art. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 11 (below).

TABLE 11
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-n-glycero-3-phosphatidylethanolamine | DOPE | Helper |
| Cholesterol | | Helper |
| N-[1-(2,3-Dioleyloxy)prophyl]N,N,N-trimethylammonium | DOTMA | Cationic |
| chloride | | |
| 1,2-Dioleoyloxy-3-trimethylammonium-propane | DOTAP | Cationic |
| Dioctadecylamidoglycylspermine | DOGS | Cationic |
| N-(3-Aminopropy1)-N,N-dimethyl-2,3-bis(dodecyloxy)-1- | GAP-DLRIE | Cationic |
| propanaminium bromide | | |
| Cetyltrimethyl ammonium bromide | CTAB | Cationic |
| 6-Lauroxyhexyl ornithinate | LHON | Cationic |
| 1-(2,3-Dioleoyl oxypropy1)-2,4,6-trimethylpyridinium | 2Oc | Cationic |
| 2,3-Dioleyloxy-N-[2(sperminecarboxamido-ethyl]-N,N- | DOSPA | Cationic |
| dimethyl-1-propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N',N'-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)] | CLIP-1 | Cationic |
| dimethylammonium chloride | | |
| rac-[2(2,3-Dihexadecyloxypropyl | CLIP-6 | Cationic |
| oxymethyloxy)ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O'-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethy1-2-heptadeceny1-3 hydroxyethyl] | DOTIM | Cationic |
| imidazolinium chloride | | |
| N1-Cholesteryloxycarbony1-3,7-diazanonane-1,9-diamine | CDAN | Cationic |
| 2-(3-[Bis(3-amino-propyl)-amino]propylamino)-N | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Table 12 lists exemplary polymers for use in gene transfer and/or nanoparticle formulations.

TABLE 12
Polymers Used for Gene Transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3'-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PANIAM |
| Poly(amidoethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | bromide) |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Table 13 summarizes delivery methods for a polynucleotide encoding a fusion protein described herein.

TABLE 13

| | | Delivery into | | | Type of |
|---|---|---|---|---|---|
| | | Non-Dividing | Duration of | Genome | Molecule |
| Delivery | Vector/Mode | Cells | Expression | Integration | Delivered |
| Physical | (e.g., | YES | Transient | NO | Nucleic Acids |
| | electroporation, | | | | and Proteins |
| | particle gun, | | | | |
| | Calcium | | | | |
| | Phosphate | | | | |
| | transfection | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno- | YES | Stable | NO | DNA |
| | Associated | | | | |
| | Virus (AAV) | | | | |
| | Vaccinia Virus | YES | Very | NO | DNA |
| | | | Transient | | |
| Herpes Simplex | YES | Stable | NO | DNA | |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

In another aspect, the delivery of genome editing system components or nucleic acids encoding such components, for example, a nucleic acid binding protein such as, for example, Cas9 or variants thereof, and a gRNA targeting a genomic nucleic acid sequence of interest, may be accomplished by delivering a ribonucleoprotein (RNP) to cells. The RNP comprises the nucleic acid binding protein, e.g., Cas9, in complex with the targeting gRNA. RNPs may be delivered to cells using known methods, such as electroporation, nucleofection, or cationic lipid-mediated methods, for example, as reported by Zuris, J. A. et al., 2015, Nat. Biotechnology, 33(1):73-80. RNPs are advantageous for use in CRISPR base editing systems, particularly for cells that are difficult to transfect, such as primary cells. In addition, RNPs can also alleviate difficulties that may occur with protein expression in cells, especially when eukaryotic promoters, e.g., CMV or EF1A, which may be used in CRISPR plasmids, are not well-expressed. Advantageously, the use of RNPs does not require the delivery of foreign DNA into cells. Moreover, because an RNP comprising a nucleic acid binding protein and gRNA complex is degraded over time, the use of RNPs has the potential to limit off-target effects. In a manner similar to that for plasmid based techniques, RNPs can be used to deliver binding protein (e.g., Cas9 variants) and to direct homology directed repair (HDR).

A promoter used to drive base editor coding nucleic acid molecule expression can include AAV ITR. This can be advantageous for eliminating the need for an additional promoter element, which can take up space in the vector. The additional space freed up can be used to drive the expression of additional elements, such as a guide nucleic acid or a selectable marker. ITR activity is relatively weak, so it can be used to reduce potential toxicity due to over expression of the chosen nuclease.

Any suitable promoter can be used to drive expression of the base editor and, where appropriate, the guide nucleic acid. For ubiquitous expression, promoters that can be used include CMV, CAG, CBh, PGK, SV40, Ferritin heavy or light chains, etc. For brain or other CNS cell expression, suitable promoters can include: SynapsinI for all neurons, CaMKIIalpha for excitatory neurons, GAD67 or GAD65 or VGAT for GABAergic neurons, etc. For liver cell expression, suitable promoters include the Albumin promoter. For lung cell expression, suitable promoters can include SP-B. For endothelial cells, suitable promoters can include ICAM. For hematopoietic cells suitable promoters can include IFNbeta or CD45. For Osteoblasts suitable promoters can include OG-2.

In some cases, a base editor of the present disclosure is of small enough size to allow separate promoters to drive expression of the base editor and a compatible guide nucleic acid within the same nucleic acid molecule. For instance, a vector or viral vector can comprise a first promoter operably linked to a nucleic acid encoding the base editor and a second promoter operably linked to the guide nucleic acid.

The promoter used to drive expression of a guide nucleic acid can include: Pol III promoters such as U6 or H1 Use of Pol II promoter and intronic cassettes to express gRNA Adeno Associated Virus (AAV).

A base editor described herein with or without one or more guide nucleic can be delivered using adeno associated virus (AAV), lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as in U.S. Pat. No. 8,454,972 and as in clinical trials involving AAV. For Adenovirus, the route of administration, formulation and dose can be as in U.S. Pat. No. 8,404,658 and as in clinical trials involving adenovirus. For plasmid delivery, the route of administration, formulation and dose can be as in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. The viral vectors can be injected into the tissue of interest. For cell-type specific base editing, the expression of the base editor and optional guide nucleic acid can be driven by a cell-type specific promoter.

For in vivo delivery, AAV can be advantageous over other viral vectors. In some cases, AAV allows low toxicity, which can be due to the purification method not requiring ultra-centrifugation of cell particles that can activate the immune response. In some cases, AAV allows low probability of causing insertional mutagenesis because it doesn't integrate into the host genome.

AAV has a packaging limit of 4.5 or 4.75 Kb. This means disclosed base editor as well as a promoter and transcription terminator can fit into a single viral vector. Constructs larger than 4.5 or 4.75 Kb can lead to significantly reduced virus production. For example, SpCas9 is quite large, the gene itself is over 4.1 Kb, which makes it difficult for packing into AAV. Therefore, embodiments of the present disclosure include utilizing a disclosed base editor which is shorter in length than conventional base editors. In some examples, the base editors are less than 4 kb. Disclosed base editors can be less than 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb. In some cases, the disclosed base editors are 4.5 kb or less in length.

An AAV can be AAV1, AAV2, AAV5 or any combination thereof. One can select the type of AAV with regard to the cells to be targeted; e.g., one can select AAV serotypes 1, 2, 5 or a hybrid capsid AAV1, AAV2, AAV5 or any combination thereof for targeting brain or neuronal cells; and one can select AAV4 for targeting cardiac tissue. AAV8 is useful for delivery to the liver. A tabulation of certain AAV serotypes as to these cells can be found in Grimm, D. et al, J. Virol. 82: 5887-5911 (2008)).

Lentiviruses are complex retroviruses that have the ability to infect and express their genes in both mitotic and post-mitotic cells. The most commonly known lentivirus is the human immunodeficiency virus (HIV), which uses the envelope glycoproteins of other viruses to target a broad range of cell types.

Lentiviruses can be prepared as follows. After cloning pCasES10 (which contains a lentiviral transfer plasmid backbone), HEK293FT at low passage (p=5) were seeded in a T-75 flask to 50% confluence the day before transfection in DMEM with 10% fetal bovine serum and without antibiotics. After 20 hours, media is changed to OptiMEM (serum-free) media and transfection was done 4 hours later. Cells are transfected with 10 μg of lentiviral transfer plasmid (pCasES10) and the following packaging plasmids: 5 μg of pMD2.G (VSV-g pseudotype), and 7.5 μg of psPAX2 (gag/pol/rev/tat). Transfection can be done in 4 mL OptiMEM with a cationic lipid delivery agent (50 μl Lipofectamine 2000 and 100 ul Plus reagent). After 6 hours, the media is changed to antibiotic-free DMEM with 10% fetal bovine serum. These methods use serum during cell culture, but serum-free methods are preferred.

Lentivirus can be purified as follows. Viral supernatants are harvested after 48 hours. Supernatants are first cleared of debris and filtered through a 0.45 μm low protein binding (PVDF) filter. They are then spun in an ultracentrifuge for 2 hours at 24,000 rpm. Viral pellets are resuspended in 50 μl of DMEM overnight at 4° C. They are then aliquoted and immediately frozen at −80° C.

In another embodiment, minimal non-primate lentiviral vectors based on the equine infectious anemia virus (EIAV) are also contemplated. In another embodiment, RETINOSTAT®, an equine infectious anemia virus-based lentiviral gene therapy vector that expresses angiostatic proteins endostatin and angiostatin that is contemplated to be delivered via a subretinal injection. In another embodiment, use of self-inactivating lentiviral vectors is contemplated.

Any RNA of the systems, for example a guide RNA or a base editor-encoding mRNA, can be delivered in the form of RNA. Base editor-encoding mRNA can be generated using in vitro transcription. For example, nuclease mRNA can be synthesized using a PCR cassette containing the following elements: T7 promoter, optional kozak sequence (GCCACC), nuclease sequence, and 3′ UTR such as a 3′ UTR from beta globin-polyA tail. The cassette can be used for transcription by T7 polymerase. Guide polynucleotides (e.g., gRNA) can also be transcribed using in vitro transcription from a cassette containing a T7 promoter, followed by the sequence “GG”, and guide polynucleotide sequence.

To enhance expression and reduce possible toxicity, the base editor-coding sequence and/or the guide nucleic acid can be modified to include one or more modified nucleoside e.g. using pseudo-U or 5-Methyl-C.

The disclosure in some embodiments comprehends a method of modifying a cell or organism. The cell can be a prokaryotic cell or a eukaryotic cell. The cell can be a mammalian cell. The mammalian cell many be a non-human primate, bovine, porcine, rodent or mouse cell. The modification introduced to the cell by the base editors, compositions and methods of the present disclosure can be such that the cell and progeny of the cell are altered for improved production of biologic products such as an antibody, starch, alcohol or other desired cellular output. The modification introduced to the cell by the methods of the present disclosure can be such that the cell and progeny of the cell include an alteration that changes the biologic product produced.

The system can comprise one or more different vectors. In an aspect, the base editor is codon optimized for expression the desired cell type, preferentially a eukaryotic cell, preferably a mammalian cell or a human cell.

In general, codon optimization refers to a process of modifying a nucleic acid sequence for enhanced expression in the host cells of interest by replacing at least one codon (e.g. about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at www.kazusa.orjp/codon/ (visited Jul. 9, 2002), and these tables can be adapted in a number of ways. See, Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, Pa.), are also available. In some embodiments, one or more codons (e.g. 1, 2, 3, 4, 5, 10, 15, 20, 25, 50, or more, or all codons) in a sequence encoding an engineered nuclease correspond to the most frequently used codon for a particular amino acid.

Packaging cells are typically used to form virus particles that are capable of infecting a host cell. Such cells include 293 cells, which package adenovirus, and psi.2 cells or PA317 cells, which package retrovirus. Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA can be packaged in a cell line, which contains a helper plasmid encoding the other AAV genes, namely rep and cap, but lacking ITR sequences. The cell line can also be infected with adenovirus as a helper. The helper virus can promote replication of the AAV vector and expression of AAV genes from the helper plasmid. The helper plasmid in some cases is not packaged in significant amounts due to a lack of ITR sequences. Contamination with adenovirus can be reduced by, e.g., heat treatment to which adenovirus is more sensitive than AAV.

### Inteins

In some embodiments, a portion or fragment of a nuclease (e.g., Cas9) is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

Inteins (intervening protein) are auto-processing domains found in a variety of diverse organisms, which carry out a process known as protein splicing. Protein splicing is a multi-step biochemical reaction comprised of both the cleavage and formation of peptide bonds. While the endogenous substrates of protein splicing are proteins found in intein-containing organisms, inteins can also be used to chemically manipulate virtually any polypeptide backbone.

In protein splicing, the intein excises itself out of a precursor polypeptide by cleaving two peptide bonds, thereby ligating the flanking extein (external protein) sequences via the formation of a new peptide bond. This rearrangement occurs post-translationally (or possibly co-translationally). Intein-mediated protein splicing occurs spontaneously, requiring only the folding of the intein domain.

About 5% of inteins are split inteins, which are transcribed and translated as two separate polypeptides, the N-intein and C-intein, each fused to one extein. Upon translation, the intein fragments spontaneously and non-covalently assemble into the canonical intein structure to carry out protein splicing in trans. The mechanism of protein splicing entails a series of acyl-transfer reactions that result in the cleavage of two peptide bonds at the intein-extein junctions and the formation of a new peptide bond between the N- and C-exteins. This process is initiated by activation of the peptide bond joining the N-extein and the N-terminus of the intein. Virtually all inteins have a cysteine or serine at their N-terminus that attacks the carbonyl carbon of the C-terminal N-extein residue. This N to O/S acyl-shift is facilitated by a conserved threonine and histidine (referred to as the TXXH motif), along with a commonly found aspartate, which results in the formation of a linear (thio)ester intermediate. Next, this intermediate is subject to trans-(thio)esterification by nucleophilic attack of the first C-extein residue (+1), which is a cysteine, serine, or threonine. The resulting branched (thio)ester intermediate is resolved through a unique transformation: cyclization of the highly conserved C-terminal asparagine of the intein. This process is facilitated by the histidine (found in a highly conserved HNF motif) and the penultimate histidine and may also involve the aspartate. This succinimide formation reaction excises the intein from the reactive complex and leaves behind the exteins attached through a non-peptidic linkage. This structure rapidly rearranges into a stable peptide bond in an intein-independent fashion.

In some embodiments, an N-terminal fragment of a base editor (e.g., ABE, CBE) is fused to a split intein-N and a C-terminal fragment is fused to a split intein-C. These fragments are then packaged into two or more AAV vectors. The use of certain inteins for joining heterologous protein fragments is described, for example, in Wood et al., J. Biol. Chem. 289(21); 14512-9 (2014). For example, when fused to separate protein fragments, the inteins IntN and IntC recognize each other, splice themselves out and simultaneously ligate the flanking N- and C-terminal exteins of the protein fragments to which they were fused, thereby reconstituting a full-length protein from the two protein fragments. Other suitable inteins will be apparent to a person of skill in the art.

In some embodiments, an ABE was split into N- and C-terminal fragments at Ala, Ser, Thr, or Cys residues within selected regions of SpCas9. These regions correspond to loop regions identified by Cas9 crystal structure analysis. The N-terminus of each fragment is fused to an intein-N and the C-terminus of each fragment is fused to an intein C at amino acid positions S303, T310, T313, S355, A456, S460, A463, T466, S469, T472, T474, C574, S577, A589, and S590, which are indicated in bold capital letters in the sequence below.

| | |
|---|---|
| 1 | mdkkysigld igtnsvgwav itdeykvpsk kfkvlgntdr hsikknliga llfdsgetae |
| 61 | atrlkrtarr rytrrknric ylqeifsnem akvddsffhr leesflveed kkherhpifg |
| 121 | nivdevayhe kyptiyhlrk klvdstdkad lrliylalah mikfrghfli egdlnpdnsd |
| 181 | vdklfiqlvg tynqlfeenp inasgvdaka ilsarlsksr rlenliaqlp gekknglfgn |
| 241 | lialslgltp nfksnfdlae daklqlskdt ydddldnlla qigdqyadlf laaknlsdai |
| 301 | llSdilrvnT eiTkaplsas mikrydehhq dltllkalvr qqlpekykei ffdqSkngya |
| 361 | gyidggasqe efykfikpil ekmdgteell vklnredllr kqrtfdngsi phqihlgelh |
| 421 | ailrrqedfy pflkdnreki ekiltfripy yvgplArgnS rfAwmTrkSe eTiTpwnfee |
| 481 | vvdkgasaqs fiermtnfdk nlpnekvlpk hsllyeyftv yneltkvkyv tegmrkpafl |
| 541 | sgeqkkaivd llfktnrkvt vkqlkedyfk kieCfdSvei sgvedrfnAS lgtyhdllki |
| 601 | ikdkdfldne enedilediv ltltlfedre mieerlktya hlfddkvmkq lkrrrytgwg |
| 661 | rlsrklingi rdkqsgktil dflksdgfan rnfmqlihdd sltfkediqk aqvsgqgdsl |
| 721 | hehianlags paikkgilqt vkvvdelvkv mgrhkpeniv iemarenqtt qkgqknsrer |
| 781 | mkrieegike lgsqilkehp ventqlqnek lylyylqngr dmyvdgeldi nrlsdydvdh |
| 841 | ivpqsflkdd sidnkvltrs dknrgksdnv pseevvkkmk nywrqllnak litqrkfdnl |
| 901 | tkaergglse ldkagfikrq lvetrqitkh vaqildsrmn tkydendkli revkvitlks |
| 961 | klvsdfrkdf qfykvreinn yhhandayln avvgtalikk ypklesefvy gdykvydvrk |
| 1021 | miakseqeig katakyffys nimnffktei tlangeirkr plietngetg eivwdkgrdf |
| 1081 | atvrkvlsmp qvnivkktev qtggfskesi lpkrnsdkli arkkdwdpkk yggfdsptva |
| 1141 | ysvlvvakve kgkskklksv kellgitime rssfeknpid fleakgykev kkdliiklpk |
| 1201 | yslfelengr krmlasagel qkgnelalps kyvnflylas hyeklkgspe dneqkqlfve |
| 1261 | qhkhyldeii eqisefskrv iladanldkv lsaynkhrdk pireqaenii hlftltnlga |
| 1321 | paafkyfdtt idrkrytstk evldatlihq sitglyetri dlsqlggd |

### Pharmaceutical Compositions

Other aspects of the present disclosure relate to pharmaceutical compositions comprising any of the base editors, fusion proteins, or the fusion protein-guide polynucleotide complexes described herein. The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents (e.g., for specific delivery, increasing half-life, or other therapeutic compounds).

As used here, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.).

Some nonlimiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum alcohols, such as ethanol; and (23) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient,” “carrier,” “pharmaceutically acceptable carrier,” “vehicle,” or the like are used interchangeably herein.

Pharmaceutical compositions can comprise one or more pH buffering compounds to maintain the pH of the formulation at a predetermined level that reflects physiological pH, such as in the range of about 5.0 to about 8.0. The pH buffering compound used in the aqueous liquid formulation can be an amino acid or mixture of amino acids, such as histidine or a mixture of amino acids such as histidine and glycine. Alternatively, the pH buffering compound is preferably an agent which maintains the pH of the formulation at a predetermined level, such as in the range of about 5.0 to about 8.0, and which does not chelate calcium ions. Illustrative examples of such pH buffering compounds include, but are not limited to, imidazole and acetate ions. The pH buffering compound may be present in any amount suitable to maintain the pH of the formulation at a predetermined level.

Pharmaceutical compositions can also contain one or more osmotic modulating agents, i.e., a compound that modulates the osmotic properties (e.g, tonicity, osmolality, and/or osmotic pressure) of the formulation to a level that is acceptable to the blood stream and blood cells of recipient individuals. The osmotic modulating agent can be an agent that does not chelate calcium ions. The osmotic modulating agent can be any compound known or available to those skilled in the art that modulates the osmotic properties of the formulation. One skilled in the art may empirically determine the suitability of a given osmotic modulating agent for use in the inventive formulation. Illustrative examples of suitable types of osmotic modulating agents include, but are not limited to: salts, such as sodium chloride and sodium acetate; sugars, such as sucrose, dextrose, and mannitol; amino acids, such as glycine; and mixtures of one or more of these agents and/or types of agents. The osmotic modulating agent(s) may be present in any concentration sufficient to modulate the osmotic properties of the formulation.

In some embodiments, the pharmaceutical composition is formulated for delivery to a subject, e.g., for gene editing. Suitable routes of administrating the pharmaceutical composition described herein include, without limitation: topical, subcutaneous, transdermal, intradermal, intralesional, intraarticular, intraperitoneal, intravesical, transmucosal, gingival, intradental, intracochlear, transtympanic, intraorgan, epidural, intrathecal, intramuscular, intravenous, intravascular, intraosseus, periocular, intratumoral, intracerebral, and intracerebroventricular administration.

In some embodiments, the pharmaceutical composition described herein is administered locally. In some embodiments, the pharmaceutical composition described herein is administered to a subject by injection, by means of a catheter, by means of a suppository, or by means of an implant, the implant being of a porous, non-porous, or gelatinous material, including a membrane, such as a sialastic membrane, or a fiber.

In other embodiments, the pharmaceutical composition described herein is delivered in a controlled release system. In one embodiment, a pump can be used (See, e.g., Langer, 1990, Science 249:1527-1533; Sefton, 1989, CRC Crit. Ref. Biomed. Eng. 14:201; Buchwald et al., 1980, Surgery 88:507; Saudek et al., 1989, N. Engl. J. Med. 321:574). In another embodiment, polymeric materials can be used. (See, e.g., Medical Applications of Controlled Release (Langer and Wise eds., CRC Press, Boca Raton, Fla., 1974); Controlled Drug Bioavailability, Drug Product Design and Performance (Smolen and Ball eds., Wiley, New York, 1984); Ranger and Peppas, 1983, Macromol. Sci. Rev. Macromol. Chem. 23:61. See also Levy et al., 1985, Science 228:190; During et al., 1989, Ann. Neurol. 25:351; Howard et ah, 1989, J. Neurosurg. 71:105.) Other controlled release systems are discussed, for example, in Langer, supra.

In some embodiments, the pharmaceutical composition is formulated in accordance with routine procedures as a composition adapted for intravenous or subcutaneous administration to a subject, e.g., a human. In some embodiments, pharmaceutical composition for administration by injection are solutions in sterile isotonic use as solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the pharmaceutical is to be administered by infusion, it can be dispensed with an infusion bottle containing sterile pharmaceutical grade water or saline. Where the pharmaceutical composition is administered by injection, an ampoule of sterile water for injection or saline can be provided so that the ingredients can be mixed prior to administration.

A pharmaceutical composition for systemic administration can be a liquid, e.g., sterile saline, lactated Ringer's or Hank's solution. In addition, the pharmaceutical composition can be in solid forms and re-dissolved or suspended immediately prior to use. Lyophilized forms are also contemplated. The pharmaceutical composition can be contained within a lipid particle or vesicle, such as a liposome or microcrystal, which is also suitable for parenteral administration. The particles can be of any suitable structure, such as unilamellar or plurilamellar, so long as compositions are contained therein. Compounds can be entrapped in “stabilized plasmid-lipid particles” (SPLP) containing the fusogenic lipid dioleoylphosphatidylethanolamine (DOPE), low levels (5-10 mol %) of cationic lipid, and stabilized by a polyethyleneglycol (PEG) coating (Zhang Y. P. et ah, Gene Ther. 1999, 6: 1438-47). Positively charged lipids such as N-[1-(2,3-dioleoyloxi)propyl]-N,N,N-trimethyl-amoniummethylsulfate, or “DOTAP,” are particularly preferred for such particles and vesicles. The preparation of such lipid particles is well known. See, e.g., U.S. Pat. Nos. 4,880,635; 4,906,477; 4,911,928; 4,917,951; 4,920,016; and 4,921,757; each of which is incorporated herein by reference.

The pharmaceutical composition described herein can be administered or packaged as a unit dose, for example. The term “unit dose” when used in reference to a pharmaceutical composition of the present disclosure refers to physically discrete units suitable as unitary dosage for the subject, each unit containing a predetermined quantity of active material calculated to produce the desired therapeutic effect in association with the required diluent; i.e., carrier, or vehicle.

Further, the pharmaceutical composition can be provided as a pharmaceutical kit comprising (a) a container containing a compound of the invention in lyophilized form and (b) a second container containing a pharmaceutically acceptable diluent (e.g., sterile used for reconstitution or dilution of the lyophilized compound of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

In another aspect, an article of manufacture containing materials useful for the treatment of the diseases described above is included. In some embodiments, the article of manufacture comprises a container and a label. Suitable containers include, for example, bottles, vials, syringes, and test tubes. The containers can be formed from a variety of materials such as glass or plastic. In some embodiments, the container holds a composition that is effective for treating a disease described herein and can have a sterile access port. For example, the container can be an intravenous solution bag or a vial having a stopper pierceable by a hypodermic injection needle. The active agent in the composition is a compound of the invention. In some embodiments, the label on or associated with the container indicates that the composition is used for treating the disease of choice. The article of manufacture can further comprise a second container comprising a pharmaceutically-acceptable buffer, such as phosphate-buffered saline, Ringer's solution, or dextrose solution. It can further include other materials desirable from a commercial and user standpoint, including other buffers, diluents, filters, needles, syringes, and package inserts with instructions for use.

In some embodiments, any of the fusion proteins, gRNAs, and/or complexes described herein are provided as part of a pharmaceutical composition. In some embodiments, the pharmaceutical composition comprises any of the fusion proteins provided herein. In some embodiments, the pharmaceutical composition comprises any of the complexes provided herein. In some embodiments, the pharmaceutical composition comprises a ribonucleoprotein complex comprising an RNA-guided nuclease (e.g., Cas9) that forms a complex with a gRNA and a cationic lipid. In some embodiments pharmaceutical composition comprises a gRNA, a nucleic acid programmable DNA binding protein, a cationic lipid, and a pharmaceutically acceptable excipient. Pharmaceutical compositions can optionally comprise one or more additional therapeutically active substances.

### Use of Nucleobase Editors to Target Nucleotides in HBV ORFS

The suitability of nucleobase editors that target a nucleotide in an HBV ORF is evaluated as described herein. In one embodiment, a single cell of interest (e.g., an animal cell infected with HBV) is transfected, transduced, or otherwise modified with a nucleic acid molecule or molecules encoding a nucleobase editor described herein together with a small amount of a vector encoding a reporter (e.g., GFP). These cells can be immortalized human cell lines, such as 293T, K562 or U20S. Alternatively, primary human cells may be used. Such cells may be relevant to the eventual cell target.

Delivery may be performed using a viral vector. In one embodiment, transfection may be performed using lipid transfection (such as Lipofectamine or Fugene) or by electroporation. Following transfection, expression of GFP can be determined either by fluorescence microscopy or by flow cytometry to confirm consistent and high levels of transfection. These preliminary transfections can comprise different nucleobase editors to determine which combinations of editors give the greatest activity.

The activity of the nucleobase editor is assessed as described herein, i.e., by sequencing the genome or ORFs of HBV to detect alterations in a target sequence. For Sanger sequencing, purified PCR amplicons are cloned into a plasmid backbone, transformed, miniprepped and sequenced with a single primer. Sequencing may also be performed using next generation sequencing techniques. When using next generation sequencing, amplicons may be 300-500 bp with the intended cut site placed asymmetrically. Following PCR, next generation sequencing adapters and barcodes (for example Illumina multiplex adapters and indexes) may be added to the ends of the amplicon, e.g., for use in high throughput sequencing (for example on an Illumina MiSeq).

The fusion proteins that induce the greatest levels of target specific alterations in initial tests can be selected for further evaluation.

In particular embodiments, the nucleobase editors are used to target polynucleotides of interest. In one embodiment, a nucleobase editor of the invention is delivered to cells (e.g., liver) in conjunction with a guide RNA that is used to target a nucleic acid sequence within the HBV genome, thereby altering the HBV gene.

In some embodiments, a base editor is targeted by a guide RNA to introduce one or more missense mutations or premature stop codons in the polymerase (pol) gene of the HBV genome. In some embodiments, the one or more mutations introduced into the pol gene by the base editor encode E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, and L719P. In some embodiments, the one or more mutations introduced into the pol gene are mutations selected from the group consisting of E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, and L719P.

In some embodiments, a base editor as described herein is targeted by a guide RNA to introduce one or more missense mutations or premature stop codons in the core gene of the HBV genome. In some embodiments, the one or more mutations introduced into the core gene comprise T160A, T160A, P161F, S162L, C183R, and/or *(STOP)184Q. In some embodiments, the one or more mutations introduced into the core gene are selected from the group consisting of T160A, T160A, P161F, S162L, C183R, and *(STOP)184Q.

In some embodiments, a base editor as described herein is targeted by a guide RNA to introduce one or more missense mutations or premature stop codons into the X gene of the HBV genome. In some embodiments, the one or more mutations introduced into the X gene comprise H86Y, R87*(STOP), H86R, W120R, W120STOP, E122K, E121K, and/or L141P. In some embodiments, the one or more mutations introduced into the core gene are selected from the group consisting of H86Y, R87*(STOP), H86R, W120R, W120STOP, E122K, E121K, and L141P.

In some embodiments, a base editor as described herein is targeted by a guide RNA to introduce one or more missense mutations or premature stop codons into the S gene of the HBV genome. In some embodiments, the one or more mutations comprise S38F, L39F, W35STOP, W35R, W36STOP, W36R, T37I, T37A, R78Q, S34L, F80P, and/or D33G. In some embodiments, the one or more mutations introduced into the S gene are selected from the group consisting of S38F, L39F, W35*(STOP), W35R, W36*(STOP), W36R, T37I, T37A, R78Q, S34L, F80P, and D33G.

### Kits

Various aspects of this disclosure provide kits comprising a base editor system. In one embodiment, the kit comprises a nucleic acid construct comprising a nucleotide sequence encoding a nucleobase editor fusion protein. The fusion protein comprises a deaminase (e.g., cytidine deaminase or adenine deaminase) and a nucleic acid programmable DNA binding protein (napDNAbp). In some embodiments, the kit comprises at least one guide RNA capable of targeting an HBV gene. In some embodiments, the kit comprises a nucleic acid construct comprising a nucleotide sequence encoding at least one guide RNA.

The kit provides, in some embodiments, instructions for using the kit to edit one or more genes in the HBV genome. The instructions will generally include information about the use of the kit for the editing nucleic acids and especially viral nucleic acids. In other embodiments, the instructions include at least one of the following: precautions; warnings; clinical studies; and/or references. The instructions may be printed directly on the container (when present), or as a label applied to the container, or as a separate sheet, pamphlet, card, or folder supplied in or with the container. In a further embodiment, a kit can comprise instructions in the form of a label or separate insert (package insert) for suitable operational parameters. In yet another embodiment, the kit can comprise one or more containers with appropriate positive and negative controls or control samples, to be used as standard(s) for detection, calibration, or normalization.

In certain embodiments, the kit is useful for the treatment of a subject having an HBV infection.

### Methods of Treating HBV Infection

The present invention provides methods of treating an HBV infection or symptoms thereof that comprise administering to a subject (e.g., a mammal, such as a human) a therapeutically effective amount of a pharmaceutical composition that comprises a polynucleotide encoding a base editor system (e.g., base editor and gRNA) described herein. In some embodiments, the base editor is a fusion protein that comprises a polynucleotide programmable DNA binding domain and an adenosine deaminase domain or a cytidine deaminase domain. A cell of the subject is transduced with the base editor and one or more guide polynucleotides that target the base editor to effect an A·T to G·C alteration (if the cell is transduced with an adenosine deaminase domain) or a C·G to U·A alteration (if the cell is transduced with a cytidine deaminase domain) of a nucleic acid sequence encoding an HBV polypeptide.

In some embodiments, treatment of chronic Hepatitis B includes a combination of approaches. For example, a subject infected with HBV can be administered a therapeutically effective amount of a pharmaceutical combination described above that targets and modifies HBV cccDNA. For example, a BE4 base editor without a UGI domain can be used with a gRNA targeting a region of the HBV genome. Without being bound by theory, omitting the inhibitor makes C->U deamination susceptible to uracil glycosylase which damages HBV cccDNA, thus destabilizing it. This treatment can be combined with a treatment that reduce or inhibit HBsAg expression (including from integrated HBV DNA), such as targeting the S or pol gene of the HBV genome using the base editors and guide RNAs described herein. Treatment can also comprise stimulating the immune system. In some embodiments, each of these three distinct therapeutic goals can be accomplished using the base editing reagents and techniques described herein.

The methods herein include administering to the subject (including a subject identified as in need of such treatment) an effective amount of a composition described herein. Identifying a subject in need of such treatment can be in the judgment of a subject or a health care professional and can be subjective (e.g. opinion) or objective (e.g. measurable by a test or diagnostic method).

The therapeutic methods of the invention in general comprise administration of a therapeutically effective amount of a pharmaceutical composition comprising, for example, a vector encoding a base editor and a gRNA that targets an HBV gene of interest to a subject (e.g., human) in need thereof. Such treatment will be suitably administered to subjects, particularly humans, suffering from, having, susceptible to, or at risk for HBV infection. The compositions herein may be also used in the treatment of any other disorders in which HBV infection may be implicated.

In one embodiment, the invention provides a method of monitoring treatment progress. The method includes the step of determining a level of diagnostic marker (Marker) (e.g., viral load) or diagnostic measurement (e.g., screen, assay) in a subject suffering from or susceptible to a disorder or symptoms thereof associated with HBV infection in which the subject has been administered a therapeutic amount of a composition herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Targeted Base Editing of the HBV Genome Resulted in Premature Stop Codons or Functional Substitutions in Viral Genes

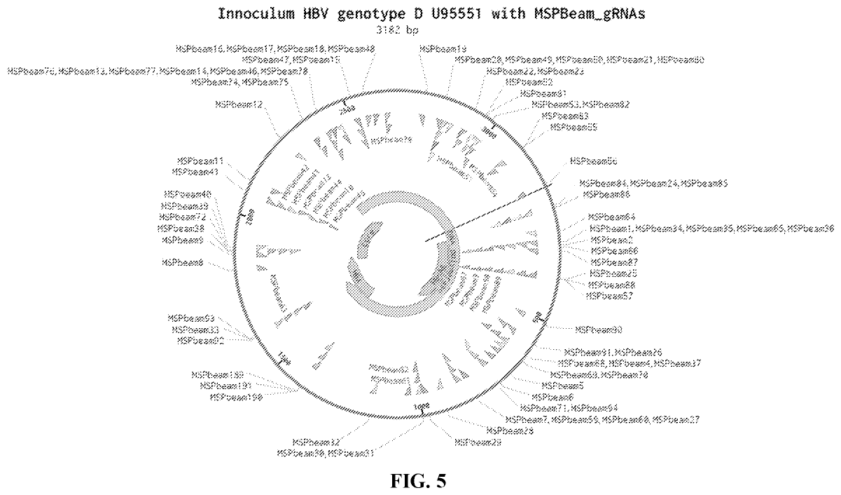

Bases editors (FIG. 4A, 4B) were evaluated for their ability to edit the HBV genome. Guide RNAs were designed to target the base editors to introduce stop codons or functional substitutions at conserved regions of HBV genes (FIG. 5). HBV Genotype D, subgenotype ayw, was analyzed using a CRISPR design tool available on Benchling Software to identify potential guide RNAs, and the level of sequence conservation across all HBV genotypes was determined for each guide RNA. 97 guide RNAs that would target a base editor to introduce a stop codon or functional change in an HBV gene based on the cytosine position in the editing window were selected for testing (Table 14). These guide RNAs were adjacent or in proximity to NGG, NAG, and NNNRRT (including NNGRRT) PAM sequences.

TABLE 14
Guide RNAs

| | | | | | | % |
|---|---|---|---|---|---|---|
| | | % Stop | Guide | | | Conservation |
| Guide | gRNA | Edit | Strand | PAM | Gene | All Genotypes |
| AATACCGCAGAGTCTAGACT | M1 | 0.47 | 1 | NNNRRT | S | 37.15943898 |
| TCCACCACGAGTCTAGACTC | M2 | 15.09 | -1 | NNNRRT | S | 96.35660164 |
| CACATCCAGCGATAACCAGG | M3 | 21.26 | -1 | NNNRRT | S | 54.84443011 |
| AAAGCCCAGGATGATGGGAT | M4 | 1.85 | -1 | NNGRRT | S | 42.35047558 |
| CGAACCACTGAACAAATGGC | M5 | 6.5 | -1 | NNNRRT | S | 92.95502176 |
| ATCCATATAACTGAAAGCCA | M6 | 28.28 | -1 | NNNRRT | S | 74.33499919 |
| ATACCCAAAGACAAAAGAAA | M7 | 3.03 | -1 | NNNRRT | S | 75.57633403 |
| ATGCAACTTTTTCACCTCTG | M8 | 0.01 | 1 | NNNRRT | Core | 94.08350798 |
| TCAAGCCTCCAAGCTGTGCC | M9 | 1.67 | 1 | NNGRRT | Core | 97.93648235 |
| CCCAGCAAAGAATTGCTTGC | M10 | 0.1 | -1 | NNGRRT | Core | 9.285829437 |
| CCACCCAGGTAGCTAGAGTC | M11 | 4.04 | -1 | NNNRRT | Core | 21.32838949 |
| AATCCACACTCCGAAAGACA | M12 | 8.95 | -1 | NNNRRT | Core | 14.78316943 |
| GTCTCAATCGCCGCGTCGCA | M13 | 9.66 | 1 | NNNRRT | Core | 68.25729486 |
| AAGATCTCAATCTCGGGAAC | M14 | 13.47 | 1 | NNNRRT | Core | 5.271642754 |
| TTTTCCAATGAGGATTAAAG | M15 | 56.89 | -1 | NNNRRT | Pol | 3.111397711 |
| TTTACACCAAGACATTATCA | M16 | 7.15 | 1 | NNNRRT | Pol | 10.23698211 |
| GAACAGTTTGTAGGCCCACT | M17 | 0.17 | 1 | NNNRRT | Pol | 3.256488796 |
| ATTGCAATTGATTATGCCTG | M18 | 0.73 | 1 | NNNRRT | Pol | 3.320973722 |
| CGCCTTCCATAGAGTGTGTA | M19 | ND | -1 | NNNRRT | Pol | 15.25068515 |
| CCCAAGAATATGGTGACCCA | M20 | 6.59 | -1 | NNNRRT | Pol | 53.32903434 |
| ACAAGATCTACAGCATGGGG | M21 | 13.57 | 1 | NNGRRT | Pol | 2.579397066 |
| CAGCCTTCAGAGCAAACACA | M22 | 18.65 | 1 | NNNRRT | Pol | 1.338062228 |
| TCAGAGCAAACACAGCAAAT | M23 | 0.55 | 1 | NNNRRT | Pol | 1.35418346 |
| CCCCAATCCTCGAGAAGATT | M24 | 0.31 | -1 | NNNRRT | Pol | 18.49105272 |
| CCAGGACAAGTTGGAGGACA | M25 | 3.1 | -1 | NNNRRT | Pol | 28.43785265 |
| GCTGTACCAAACCTTCGGAC | M26 | 12.17 | 1 | NNNRRT | Pol | 13.92874416 |
| ACCCCATCTCTTTGTTTTGT | M27 | 0.15 | -1 | NNGRRT | Pol | 4.981460584 |
| CCACAAGAACACATCATACA | M28 | 0.29 | 1 | NNNRRT | Pol | 1.837820409 |
| ACTTTCCAATCAATAGGCCT | M29 | 4.1 | -1 | NNNRRT | Pol | 2.063517653 |
| GTCAACGAATTGTGGGTCTT | M30 | 0.02 | 1 | NNGRRT | Pol | 3.981944221 |
| ACACAATGTGGTTATCCTGC | M31 | 0.17 | 1 | NNNRRT | Pol | 17.58826374 |
| CGGCAACGGCCAGGTCTGTG | M32 | 1.28 | 1 | NNNRRT | Pol | 20.13541835 |
| GTGGTCTCCATGCGACGTGC | M33 | 34.2 | -1 | NNNRRT | Pol | 68.4829921 |
| TACCGCAGAGTCTAGACTCG | M34 | 6.7 | 1 | NGG | S | 37.06271159 |
| CGCAGAGTCTAGACTCGTGG | M35 | 0.53 | 1 | NGG | S | 37.12719652 |
| CACCACGAGTCTAGACTCTG | M36 | 3.14 | -1 | NGG | S | 94.29308399 |
| GAAAGCCCAGGATGATGGGA | M37 | 16.59 | -1 | NGG | S | 40.59326133 |
| CCACCCAAGGCACAGCTTGG | M38 | 0.31 | -1 | NGG | Core | 94.53490247 |
| AAGCCACCCAAGGCACAGCT | M39 | 27.31 | -1 | NGG | Core | 94.61550862 |
| CCATGCCCCAAAGCCACCCA | M40 | 30.21 | -1 | NGG | Core | 64.59777527 |
| TCAGGCAAGCAATTCTTTGC | M41 | 4.01 | 1 | NGG | Core | 9.592132839 |
| CAGGCAAGCAATTCTTTGCT | M42 | 6.22 | 1 | NGG | Core | 9.576011607 |
| AGGCAAGCAATTCTTTGCTG | M43 | 0.91 | 1 | NGG | Core | 9.592132839 |
| GGCAAGCAATTCTTTGCTGG | M44 | 0.32 | 1 | NGG | Core | 8.189585684 |
| GCAAGCAATTCTTTGCTGGG | M45 | 0.02 | 1 | NGG | Core | 8.221828148 |
| TCCAAGGAATACTAACATTG | M46 | 50.79 | -1 | NGG | Pol | 2.950185394 |
| TTCCAATGAGGATTAAAGAC | M47 | 45.95 | -1 | NGG | Pol | 3.337094954 |
| TGCAATTGATTATGCCTGCT | M48 | 1.16 | 1 | NGG | Pol | 8.866677414 |
| TGGGAACAAGATCTACAGCA | M49 | 30.73 | 1 | NGG | Pol | 22.31178462 |
| GGGAACAAGATCTACAGCAT | M50 | 51.93 | 1 | NGG | Pol | 22.27954216 |
| GGAACAAGATCTACAGCATG | M51 | 40.48 | 1 | NGG | Pol | 3.320973722 |
| TCAATCCCAACAAGGACACC | M52 | 58.84 | 1 | NGG | Pol | 15.29904885 |
| GACGCCAACAAGGTAGGAGC | M53 | 36.32 | 1 | NGG | Pol | 15.91165565 |
| TGCTCCAGCTCCTACCTTGT | M54 | 45.57 | -1 | NGG | Pol | 16.16959536 |
| CCACCAATCGCCAGACAGGA | M55 | 27.87 | 1 | NGG | Pol | 0.709334193 |
| AGCCACCAGCAGGGAAATAC | M56 | 41.17 | -1 | NGG | Pol | 16.73383847 |
| ACCAGGACAAGTTGGAGGAC | M57 | 18.71 | -1 | NGG | Pol | 16.21795905 |
| CGATAACCAGGACAAGTTGG | M58 | 44.43 | -1 | NGG | Pol | 18.2814767 |
| CCCATCTCTTTGTTTTGTTA | M59 | 0.45 | -1 | NGG | Pol | 4.933096889 |
| CCCCATCTCTTTGTTTTGTT | M60 | 2.03 | -1 | NGG | Pol | 5.223279059 |
| TCAACGAATTGTGGGTCTTT | M61 | 5.2 | 1 | NGG | Pol | 23.48863453 |
| CAACGAATTGTGGGTCTTTT | M62 | 1.6 | 1 | NGG | Pol | 24.81057553 |
| GGTCTCCATGCGACGTGCAG | M63 | 27.5 | -1 | NGG | Pol | 68.88602289 |
| ACAGGCGGGGTTTTTCTTGT | M64 | ND | 1 | NGA | S | 86.8128325 |
| GCAGAGTCTAGACTCGTGGT | M65 | 0.02 | 1 | NGA | S | 37.19168144 |
| GAGAAGTCCACCACGAGTCT | M66 | 26.51 | -1 | NGA | S | 97.16266323 |
| GACACATCCAGCGATAACCA | M67 | 37.7 | -1 | NGA | S | 54.97339997 |
| AAGCCCAGGATGATGGGATG | M68 | 45.01 | -1 | NGA | S | 42.38271804 |
| CCACTCCCATAGGAATTTTC | M69 | 1.1 | -1 | NGA | S | 22.198936 |
| CTGAGCCAGGAGAAACGGGC | M70 | 30.01 | -1 | NGA | S | 22.10220861 |
| TACCACATCATCCATATAAC | M71 | 7.13 | -1 | NGA | 5 | 57.55279703 |
| AGCCACCCAAGGCACAGCTT | M72 | 7.39 | -1 | NGA | Core | 94.32532645 |
| CCAGCAAAGAATTGCTTGCC | M73 | 2.07 | -1 | NGA | Core | 9.576011607 |
| GACGACGAGGCAGGTCCCCT | M74 | 20.83 | 1 | NGA | Core | 52.34563921 |
| GACGAGGCAGGTCCCCTAGA | M75 | 0.63 | 1 | NGA | Core | 50.66903111 |
| GGTCTCAATCGCCGCGTCGC | M76 | 23.22 | 1 | NGA | Core | 69.57923585 |
| CTCAATCGCCGCGTCGCAGA | M77 | ND | 1 | NGA | Core | 91.76205062 |
| AGTCCAAGGAATACTAACAT | M78 | 28.77 | -1 | NGA | Pol | 32.01676608 |
| TGTTTTCCAATGAGGATTAA | M79 | 22.75 | -1 | NGA | Pol | 3.320973722 |
| TTTCCACCAGCAATCCTCTG | M80 | 7.42 | 1 | NGA | Pol | 16.18571659 |
| CCCAACAAGGACACCTGGCC | M81 | 2.41 | 1 | NGA | Pol | 15.17007899 |
| ACGCCAACAAGGTAGGAGCT | M82 | 25.55 | 1 | NGA | Pol | 16.0889892 |
| CCTCCACCAATCGCCAGACA | M83 | 8.63 | 1 | NGA | Pol | 0.693212961 |
| CCAATCCTCGAGAAGATTGA | M84 | 0.12 | -1 | NGA | Pol | 21.66693535 |
| TCCCCAATCCTCGAGAAGAT | M85 | 24.44 | -1 | NGA | Pol | 22.10220861 |
| AGGGTCCCCAATCCTCGAGA | M86 | 45.43 | -1 | NGA | Pol | 19.70014509 |
| CTTCTCTCAATTTTCTAGGG | M87 | 21.49 | 1 | NGA | Pol | 82.52458488 |
| CCAGGACAAGTTGGAGGACA | M88 | 15 | -1 | NGA | Pol | 27.63179107 |
| GATAACCAGGACAAGTTGGA | M89 | 50.24 | -1 | NGA | Pol | 18.2814767 |
| CCACCAGCACGGGACCATGC | M90 | 15.43 | 1 | NGA | Pol | 7.818797356 |
| GCTGTACCAAACCTTCGGAC | M91 | 29.59 | 1 | NGA | Pol | 14.23504756 |
| CTCCATGCGACGTGCAGAGG | M92 | 26.43 | -1 | NGA | Pol | 68.99887151 |
| GTGGTCTCCATGCGACGTGC | M93 | 33.81 | -1 | NGA | Pol | 68.4829921 |
| TATGGATGATGTGGTATTGG | M94 | 30.62 | 1 | NGG | Pol | 67.22553603 |
| TGCCAACTGGATCCTGCGCG | M189 | 0.23 | 1 | NGA | X | 72.48105755 |
| GCTGCCAACTGGATCCTGCG | M190 | 36.53 | 1 | NGG | X | 76.28566823 |
| CTGCCAACTGGATCCTGCGC | M191 | 41.43 | 1 | NGG | X | 72.49717878 |
| CGCCCACCGAATGTTGCCCA | E95 | 45.29 | 1 | NGG | X | |
| CTCCTCCCAGTCTTTAAACA | E96 | 0.01 | 1 | NNNRRT | X | |

Gene editing efficiency was evaluated as follows. For plasmid transfections, a vector encoding guide RNA and a vector encoding a base editor were transiently transfected into HEK293T cells previously transduced with a lentiviral vector comprising the HBV genome. Base editors tested included: ABE, BE4 (FIG. 4B), or a BE4 variant from among the following:

A3A-BE4 denotes a variant of BE4 where APOBEC-1 is replaced with the sequence of APOBEC-3A; APOBEC-3A (A3A) is described, for example, by Gehrke et al., Nature Biotechnology volume 36, pages 977-982 (2018);

A3A-BE4-VRQR denotes a variant of BE4 where APOBEC-1 is replaced with the sequence of APOBEC-3A, and Cas9 is replaced with a Cas9 variant comprising V1134, R1217, Q1334, and R1336 (termed SpCas9-VRQR); and

BE4-VRQR denotes a BE4 variant where Cas9 is replaced with a Cas9 variant comprising V1134, R1217, Q1334, and R1336 (termed SpCas9-VRQR). A summary of the base editors used with specific guides is provided at Table 13.

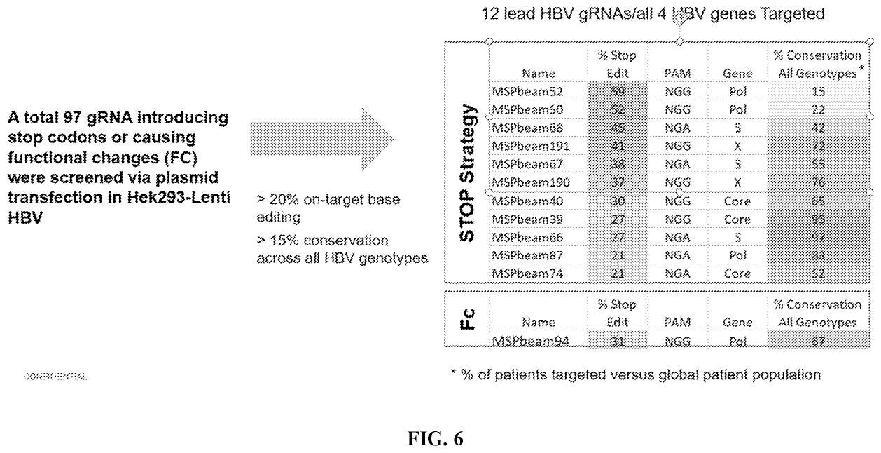

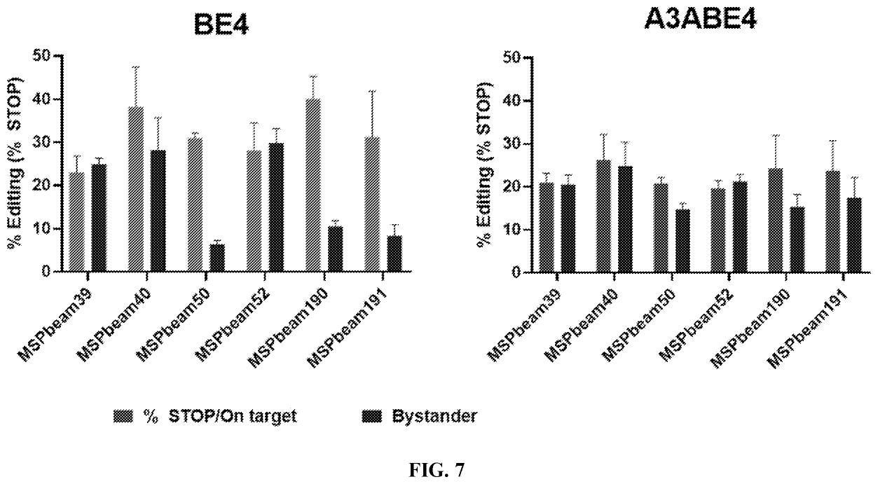

Transfection was carried out using a high efficiency, low toxicity DNA transfection reagent (Mirus TransIT293 or Lipofectamine 2000) optimized for Hek293 or HepG2-NTCP cells in a 3:1 ratio using 250 ng of gRNA plasmid and 750 ng of base editor plasmid. For mRNA transfections, HepG2-NTCP cells were transfected with in vitro transcribed base editor mRNA and synthetic gRNA in a 3:1 ratio using lipofectamin Messengermax. After four days for plasmid transfections and two days for RNA transfection, genomic DNA was extracted with a simple lysis buffer of 0.05% SDS, 25 μg/ml proteinase K, and 10 mM Tris pH 8.0, followed by heat inactivation at 85° C. Genomic sites were PCR amplified and sequenced on a MiSeq. Approximately 60% of the guide RNAs tested introduced a stop codon into the HBV genome. Of the 97 guide RNAs tested, 12 had greater than 20% on-target base editing and greater than 15% conservation across all HBV genomes (FIG. 6, Table 15 (M81 and M189 are included in the table, but were used as negative controls)).

TABLE 15
HBV Target Summary

| | Gene | | | | |
|---|---|---|---|---|---|
| Disease | Symbol | gRNA | Editor | Protospacer | PAM |
| HBV | S | M68 | BE4-VRQR or A3A-BE4-VRQR | AAGCCCAGGATGATGGGATG | NGA |
| HBV | S | M67 | BE4-VRQR or A3A-BE4-VRQR | GACACATCCAGCGATAACCA | NGA |
| HBV | S | M66 | BE4-VRQR or A3A-BE4-VRQR | GAGAAGTCCACCACGAGTCT | NGA |
| HBV | Pol | M87 | BE4-VRQR or A3A-BE4-VRQR | CTTCTCTCAATTTTCTAGGG | NGA |
| HBV | Core | M74 | BE4-VRQR or A3A-BE4-VRQR | GACGACGAGGCAGGTCCCCT | NGA |
| HBV | Pol | M81 | BE4-VRQR or A3A-BE4-VRQR | CCCAACAAGGACACCTGGCC | NGA |
| HBV | X | M189 | BE4-VRQR or A3A-BE4-VRQR | TGCCAACTGGATCCTGCGCG | NGA |
| HBV | Pol | M52 | BE4 or A3A-BE4 | TCAATCCCAACAAGGACACC | NGG |
| HBV | Pol | M50 | BE4 or A3A-BE4 | GGGAACAAGATCTACAGCAT | NGG |
| HBV | X | M191 | BE4 or A3A-BE4 | CTGCCAACTGGATCCTGCGC | NGG |
| HBV | X | M190 | BE4 or A3A-BE4 | GCTGCCAACTGGATCCTGCG | NGG |
| HBV | Pol | M94 | ABE | TATGGATGATGTGGTATTGG | NGG |
| HBV | Pre-Core | M40 | BE4 or A3A-BE4 | CCATGCCCCAAAGCCACCCA | NGG |
| HBV | Pre-Core | M39 | BE4 or A3A-BE4 | AAGCCACCCAAGGCACAGCT | NGG |

Of these 12 guide RNAs, M94 introduced a functional change mutation in the catalytic domain of the HBV polymerase (a D540G substitution). Mutations at this position are known to inhibit priming and elongation activity of the polymerase

### Example 2: Different Base Editors Introduced Alterations in the HBV Genome

Different base editors (i.e., BE4, BE4-VRQR, A3A-BE4-VRQR, and ABE (TadA7.10) were compared for efficiency and specificity. Base editing efficiency refers to the number of sequencing reads having an edited sequence divided by the total number of sequencing reads analyzed. Base editing specificity refers to the ratio of intended base edits relative to bystander base edits. Base editors and guide RNAs were introduced into cells as described above. Specifically, a subset of the 12 guide RNAs that had at least 20% on-target base editing was tested with BE4 and A3ABE4 base editors. BE4 comprises an APOBEC cytidine deaminase, a Cas9 domain, and two uracil DNA glycosylase inhibitor (UGI) domains, whereas A3A-BE4 comprises an APOBEC3A cytidine deaminase, a Cas9 domain, and two UGI domains. Referring to FIG. 7, the BE4 base editor had better efficiency for each guide RNA and better specificity for the majority of the guide RNAs compared to the base editor comprising an APOBEC3A cytidine deaminase (A3ABE4).

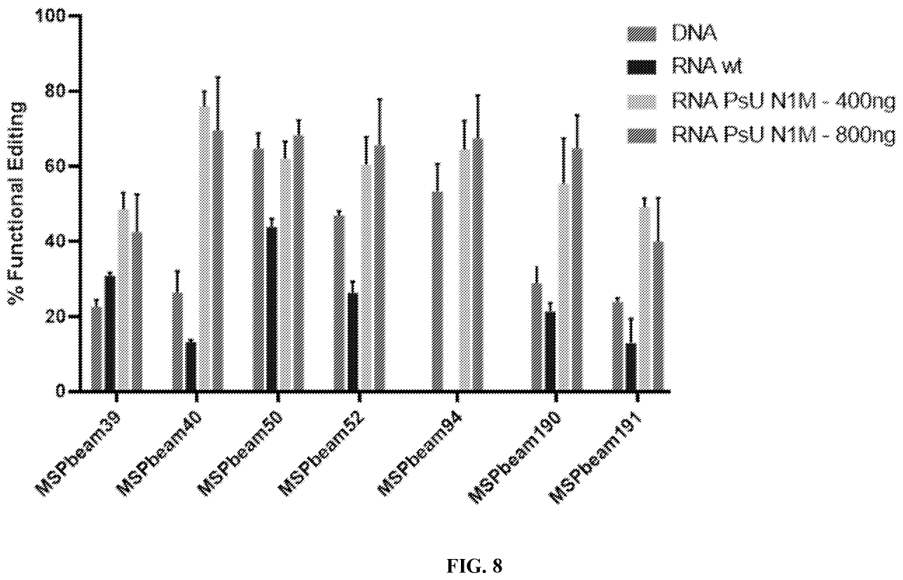

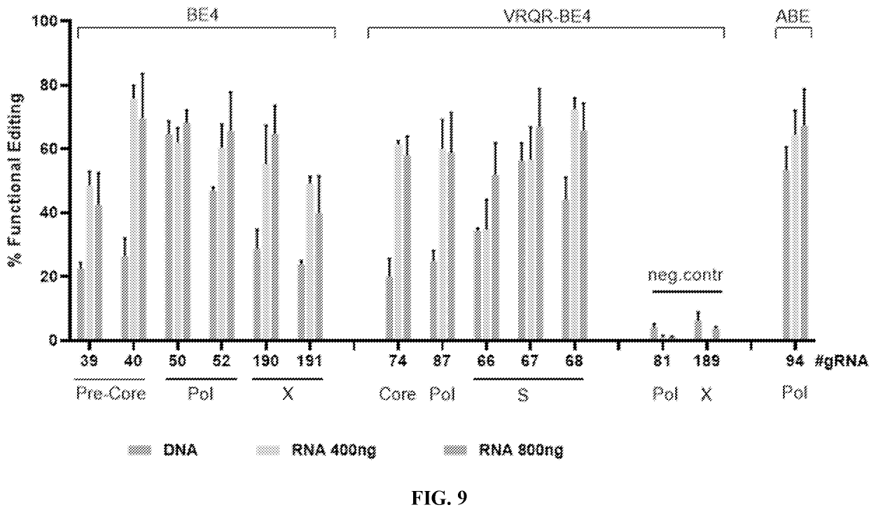

DNA and RNA constructs encoding the BE4 base editor were introduced into HepG2-NTCP-lenti-HBV cell line along with guide RNAs. Specifically, DNA constructs, wild type RNA constructs, and RNA constructs comprising pseudo-uracil modified at the N1 moiety were tested. Two different amounts (400 ng and 800 ng) of the modified RNA constructs were tested. Each construct generated base editing, but RNA constructs comprising pseudo-uracil appeared to exhibit greater base editing efficiency than did the DNA or wild type RNA constructs (FIGS. 8 and 9).

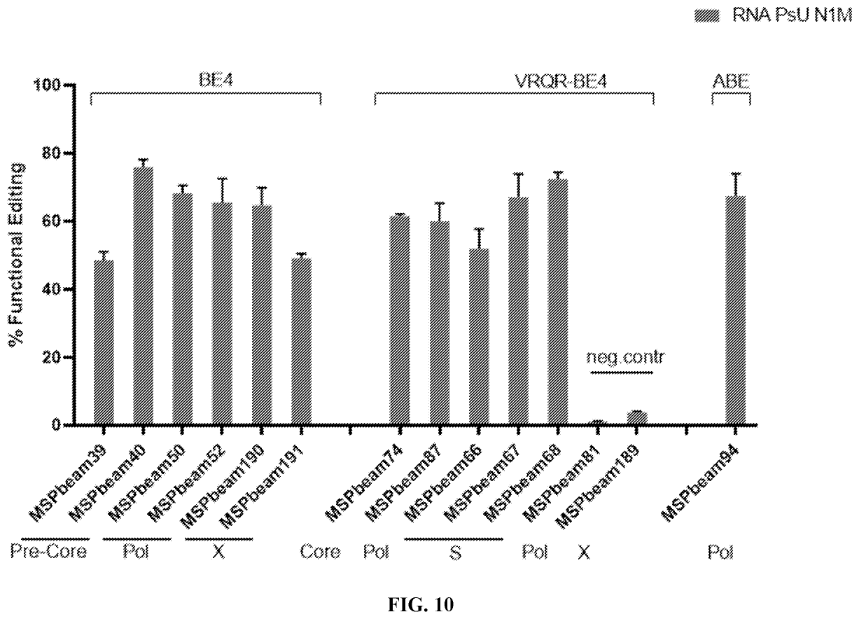

The guide RNAs were further tested with BE4, BE4-VRQR, and ABE base editors. RNA constructs comprising pseudo-uracil modified at the N1 moiety and encoding the base editors were introduced into HepG2-NTCP-lenti-HBV cell line along with guide RNAs. Referring to FIG. 10, each base editor exhibited greater than 40% functional editing. Tables 16 and 17 provide potential base editing outcomes for BE4 and ABE base editing systems.

TABLE 16
Characterization of BE4 base editing for guide RNAs selected for introducing functional
change or stop codon

| | | | | Frequency of the anticipated | No. of |
|---|---|---|---|---|---|
| | | | | amino acid change (across the | cytidines |
| | | | Mutations | sequenced HBV proteins, | in base |
| Sequence | | Guide | Introduced by BE4 | according to the Hepatitis B | editing |
| of gRNA | PAM | Id | base editors | Virus database at Hbvdb.ibcp.fr) | window |
| AAGCCACCCA | NGG | M39 | | | 4 |
| AGGCACAGCT | | | | | |
| CCATGCCCCAA | NGG | M40 | | | 3 |
| AGCCACCCA | | | | | |
| GGGAACAAGA | NGG | M50 | Pol: Q177Stop | Pol: 177* = 0.00%, Pol: 177Q = | 1 |
| TCTACAGCAT | | | | 98.74% | |
| TCAATCCCAAC | NGG | M52 | Pol: S216F, Pol: | Pol: 216F = 0.01%, Pol: 216S = | 3 |
| AAGGACACC | | | Q217Stop | 22.91%, Pol: 217* = 0.00%, | |
| | | | | Pol: 217Q = 56.79% | |
| GCTGCCAACT | NGG | M190 | X: Q8Stop, | Pol: 757A = 99.91%, Pol: 757V = | 2 |
| GGATCCTGCG | | | Pol: A757V | 0.00%, X: 8* = 0.00%, | |
| | | | | X: 8Q = 99.25% | |
| CTGCCAACTG | NGG | M191 | X: Q8Stop, | Pol: 757A = 99.91%, Pol: 757V = | 3 |
| GATCCTGCGC | | | Pol: A757V | 0.00%, X: 8* = 0.00%, | |
| | | | | X: 8Q = 99.25% | |
| GACGACGAGG | NGA | M74 | Core: R152Stop | Core: 152* = 0.00%, Core: | 1 |
| CAGGTCCCCT | | | | 152R = 99.59% | |
| CTTCTCTCAAT | NGA | M87 | S: S38F, S: L39F, | Pol: 382F = 0.05%, Pol: 382S = | 3 |
| TTTCTAGGG | | | Pol: S382F, Pol: | 99.86%, Pol: 383* = 0.00%, | |
| | | | Q383Stop | Pol: 383Q = 99.93%, S: 38F = 0.01%, | |
| | | | | S: 38S = 99.93%, S: 39F = 0.04%, | |
| | | | | S: 39L = 99.84% | |
| GAGAAGTCCA | NGA | M66 | S: W36Stop, Pol: | Pol: 380D = 99.88%, Pol: 380N = | 1 |
| CCACGAGTCT | | | D380N | 0.01%, S: 36* = 0.00%, | |
| | | | | S: 36W = 99.55% | |
| GACACATCCA | NGA | M67 | S: W74Stop, S: M75I, | Pol: 418D = 99.93%, Pol: 418N = | 2 |
| GCGATAACCA | | | Pol: D418N, Pol: | 0.01%, Pol: 419M = 0.05%, Pol: | |
| | | | V419M | 419V = 99.85%, S: 74* = 0.00%, | |
| | | | | S: 74W = 99.15%, S: 75I = 0.07%, | |
| | | | | S: 75M = 99.79% | |
| AAGCCCAGGA | NGA | M68 | S: W156Stop, S: | Pol: 500G = 99.91%, Pol: 500N = | 3 |
| TGATGGGATG | | | A157T, Pol: G500N | 0.00%, S: 156* = 0.00%, S: 156W = | |
| | | | | 99.65%, S: 157A = 99.83%, | |
| | | | | S: 157T = 0.02% | |
| CCCAACAAGG | NGA | M81 | Pol: Q218Stop | Pol: 218* = 0.00%, Pol: | 1 |
| ACACCTGGCC | | | | 218Q = 99.00% | |
| TGCCAACTGG | NGA | M189 | X: Q8Stop | X: 8* = 0.00%, X: 8Q = 99.25% | 2 |
| ATCCTGCGCG | | | | | |
| TATGGATGAT | NGG | M94 | | | 0 |
| GTGGTATTGG | | | | | |

TABLE 17
Characterization of ABE base editing for guide RNAs selected for introducing
functional change or stop codon

| | Mutations Introduced by | | No. of cytidines in base |
|---|---|---|---|
| Guide Id | BE4 base editors | Allele Frequencies | editing window |
| M39 | | | 1 |
| M40 | | | 0 |
| M50 | Pol: E176G, Pol: Q177R | Pol: 176E = 99.94%, Pol: 176G = 0.02%, | 4 |
| | | Pol: 177Q = 98.74%, Pol: 177R = 0. | |
| | | 05% | |
| M52 | | | 1 |
| M190 | X: Q8R, Pol: N758G | Pol: 758G = 0.00%, Pol: 758N = 99.78%, | 2 |
| | | X: 8Q = 99.25%, X: 8R = 0.07% | |
| M191 | X: Q8R, Pol: N758G | Pol: 758G = 0.00%, Pol: 758N = 99.78%, | 2 |
| | | X: 8Q = 99.25%, X: 8R = 0.07% | |
| M74 | Pol: D16G, Pol: E17G | Pol: 16D = 69.15%, Pol: 16G = 0.12%, | 2 |
| | | Pol: 17E = 94.58%, Pol: 17G = 0.59% | |
| M87 | | | 0 |
| M66 | S: S38P, Pol: F381P | Pol: 381F = 99.93%, Pol: 381P = 0.00%, | 2 |
| | | S: 38P = 0.04%, S: 38S = 99.93% | |
| M67 | S: M75T,S: C76R, Pol: V419A | Pol: 419A = 0.06%, Pol: 419V = 99.85%, | 2 |
| | | S: 75M = 99.79%, S: 75T = 0.06%, S: | |
| | | 76C = 96.88%, S: 76R = 0.04% | |
| M68 | S: W156R, Pol: L499P | Pol: 499L = 95.19%, Pol: 499P = 0.01%, | 1 |
| | | S: 156R = 0.02%, S: 156W = 99.65% | |
| M81 | Pol: Q217R, Pol: Q218R | Pol: 217Q = 56.79%, Pol: 217R = 0.72%, | 4 |
| | | Pol: 218Q = 99.00%, Pol: 218R = 0.01% | |
| M189 | X: Q8R, Pol: N758G | Pol: 758G = 0.00%, Pol: 758N = 99.78%, | 2 |
| | | X: 8Q = 99.25%, X: 8R = 0.07% | |
| M94 | S: M197V, Pol: D540G | Pol: 540D = 99.94%, Pol: 540G = 0.01%, | 1 |
| | | S: 197M = 99.59%, S: 197V = 0.01% | |

### Example 3: Base Editors were Used to Target Conserved Regions of the HBV Genome

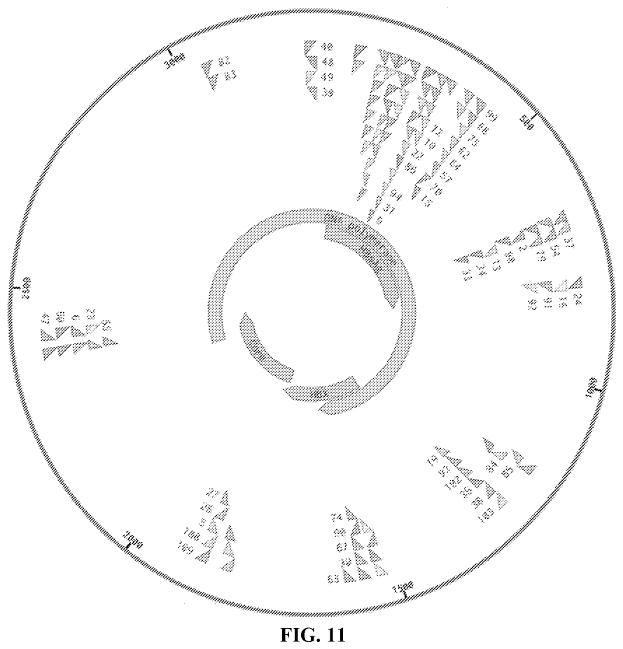

Base editors were evaluated for their ability to edit conserved regions of the HBV genome (FIG. 11). Table 18 identifies 126 guide RNAs that targeted regions conserved in >80% of 1-HBV A and D genotypes (“super-conserved”) and that were designed to minimize off-target effects (i.e., targeting a human genomic region). These guide RNAs are expected to introduce functional changes based on the high degree of conservation among HBV genomes. Without being bound by theory, targeting deaminase to HBV cccDNA using a base editor (including base editor without UGI has the potential to promote formation of apyrimidinic sites and cccDNA damage and/or degradation. Table 18 provides a list of sequences for guide RNAs that target a base editor to conserved regions of the HBV genome. These guide RNAs target sequences having greater than 50% conservation between HBV genotypes A, B, C and D and having at least one mismatch with the most similar sequence in human genome hg38, thereby minimizing off-target effects.

TABLE 18
Conserved gRNA list

| guide_seq | guide_id | pam |
|---|---|---|
| AAAAACCCCGCCTGTAACAC | Novel | NAG |
| AAAACGCCGCAGACACATCC | Novel | NGC |
| AAAGGCCTTGTAAGTTGGCG | Novel | NGA |
| AAAGGCCTTGTAAGTTGGCGA | Novel | NNNRRT |
| AACCACTGAACAAATGGCAC | Novel | NAG |
| AACCCCGCCTGTAACACGAG | Novel | NAG |
| AACGCCGCAGACACATCCAG | Novel | NGA |
| AAGAAGTCAGAAGGCAAAAA | Novel | NGA |
| AAGCCACCCAAGGCACAGCT | MSPbeam39 | NGG |
| AAGCCCTACGAACCACTGAA | Novel | NAA |
| AAGCCTCCAAGCTGTGCCTT | Novel | NGG |
| AAGGCACAGCTTGGAGGCTT | Novel | NAA |
| AAGGCACAGCTTGGAGGCTTG | EMSbeaml | NNNRRT |
| AAGGCCTTGTAAGTTGGCGA | Novel | NAA |
| AATCGCCGCGTCGCAGAAGAT | Novel | NNNRRT |
| AATGTCAACGACCGACCTTG | Novel | NGG |
| ACAGGCGGGGTTTTTCTTGT | MSPbeam64 | NGA |
| ACCAATTTATGCCTACAGCCT | EMSbeam2 | NNNRRT |
| ACGAACCACTGAACAAATGGC | MSPbeam5 | NNNRRT |
| ACGGGGCGCACCTCTCTTTA | Novel | NGC |
| ACTCCCTCGCCTCGCAGACG | Novel | NAG |
| ACTGAACAAATGGCACTAGT | Novel | NAA |
| ACTTCTCTCAATTTTCTAGG | Novel | NGG |
| ACTTTCTCGCCAACTTACAA | Novel | NGC |
| AGAAAGGCCTTGTAAGTTGG | Novel | NGA |
| AGAAGAACTCCCTCGCCTCG | Novel | NAG |
| AGACAAAAGAAAATTGGTAA | Novel | NAG |
| AGCCACCCAAGGCACAGCTT | MSPbeam72 | NGA |
| AGCCCTACGAACCACTGAAC | Novel | NAA |
| AGCTGTGCCTTGGGTGGCTT | Novel | NGG |
| AGGAGGCTGTAGGCATAAAT | EMSbeam3 | NGG |
| AGGAGTTCCGCAGTATGGAT | EMSbeam4 | NGG |
| AGGCAGGTCCCCTAGAAGAA | Novel | NAA |
| AGGCCTTGTAAGTTGGCGAG | Novel | NAA |
| AGGCGAGGGAGTTCTTCTTC | Novel | NAG |
| AGTCCAAGAGTCCTCTTATG | Novel | NAA |
| AGTTCCGCAGTATGGATCGG | Novel | NAG |
| AGTTCTTCTTCTAGGGGACC | Novel | NGC |
| ATCCATACTGCGGAACTCCT | Novel | NGC |
| ATCTTCTGCGACGCGGCGAT | Novel | NGA |
| ATGTCAACGACCGACCTTGA | Novel | NGC |
| ATTTGTTCAGTGGTTCGTAG | Novel | NGC |
| CAAATGGCACTAGTAAACTG | Novel | NGC |
| CAAGCCTCCAAGCTGTGCCT | EMSbeam5 | NGG |
| CAAGGCACAGCTTGGAGGCT | EMSbeam6 | NGA |
| CACCACGAGTCTAGACTCTG | MSPbeam36 | NGG |
| CACCCAAGGCACAGCTTGGA | Novel | NGC |
| CACTGAACAAATGGCACTAG | Novel | NAA |
| CATACTGCGGAACTCCTAGC | Novel | NGC |
| CATGCAACTTTTTCACCTCTG | MSPbeam8 | NNNRRT |
| CATGCCCCAAAGCCACCCAA | Novel | NGC |
| CCACCCAAGGCACAGCTTGG | MSPbeam38 | NGG |
| CCATGCAACTTTTTCACCTC | Novel | NGC |
| CCATGCCCCAAAGCCACCCA | MSPbeam40 | NGG |
| CCCAAAGCCACCCAAGGCAC | Novel | NGC |
| CCCCAAAGCCACCCAAGGCA | Novel | NAG |
| CCCGTCTGTGCCTTCTCATC | Novel | NGC |
| CCGCAGTATGGATCGGCAGA | EMSbeam7 | NGA |
| CCTACGAACCACTGAACAAA | Novel | NGG |
| CCTCCAAGCTGTGCCTTGGG | Novel | NGG |
| CCTCTGCCGATCCATACTGC | EMSbeam8 | NGA |
| CCTGCTGGTGGCTCCAGTTC | Novel | NGG |
| CGCCGCGTCGCAGAAGATCT | Novel | NAA |
| CGCGTAAAGAGAGGTGCGCCC | Novel | NNNRRT |
| CGTGCAGAGGTGAAGCGAAG | Novel | NGC |
| CTACGAACCACTGAACAAAT | Novel | NGC |
| CTAGACTCGTGGTGGACTTCT | EMSbeam9 | NNNRRT |
| CTCAATCGCCGCGTCGCAGA | MSPbeam77 | NGA |
| CTCCAAGCTGTGCCTTGGGT | Novel | NGC |
| CTCTGCCGATCCATACTGCG | Novel | NAA |
| CTGTGCCTTGGGTGGCTTTG | Novel | NGG |
| CTGTTCAAGCCTCCAAGCTG | Novel | NGC |
| CTTCTCTCAATTTTCTAGGG | MSPbeam87 | NGA |
| CTTCTGCGACGCGGCGATTG | Novel | NGA |
| GAAAAACCCCGCCTGTAACA | Novel | NGA |
| GAAAGCCCTACGAACCACTGA | Novel | NNNRRT |
| GAACTCCCTCGCCTCGCAGAC | EMSbeam10 | NNNRRT |
| GAACTGGAGCCACCAGCAGG | Novel | NAA |
| GAAGAACTCCCTCGCCTCGC | EMSbeam11 | NGA |
| GACAAACGGGCAACATACCTT | Novel | NNNRRT |
| GACTCGTGGTGGACTTCTCT | Novel | NAA |
| GACTTCTCTCAATTTTCTAG | EMSbeam12 | NGG |
| GAGAAGTCCACCACGAGTCT | MSPbeam66 | NGA |
| GAGGACAAACGGGCAACATAC | Novel | NNNRRT |
| GAGGCAGGTCCCCTAGAAGA | Novel | NGA |
| GATCCATACTGCGGAACTCC | Novel | NAG |
| GATTGAGATCTTCTGCGACGC | Novel | NNNRRT |
| GCAACTTTTTCACCTCTGCC | Novel | NAA |
| GCACAGCTTGGAGGCTTGAA | Novel | NAG |
| GCCACCCAAGGCACAGCTTG | Novel | NAG |
| GCGTCAGCAAACACTTGGCA | Novel | NAG |
| GCTGCTATGCCTCATCTTCTT | EMSbeam14 | NNNRRT |
| GCTGTGCCTTGGGTGGCTTT | Novel | NGG |
| GGAAAGCCCTACGAACCACT | Novel | NAA |
| GGACTTCTCTCAATTTTCTA | EMSbeam15 | NGG |
| GGAGTGCGAATCCACACTCC | Novel | NAA |
| GGAGTTCCGCAGTATGGATC | Novel | NGC |
| GGCACTAGTAAACTGAGCCA | Novel | NGA |
| GGCCTTGTAAGTTGGCGAGA | Novel | NAG |
| GGCGGGGTTTTTCTTGTTGAC | Novel | NNGRRT |
| GGCGGGGTTTTTCTTGTTGAC | Novel | NNNRRT |
| GGCGTTCACGGTGGTCTCCA | Novel | NGC |
| GGGAAAGCCCTACGAACCAC | Novel | NGA |
| GGGACTGCGAATTTTGGCCA | Novel | NGA |
| GGGGCGCACCTCTCTTTACG | Novel | NGG |
| GGGTTGCGTCAGCAAACACT | Novel | NGG |
| GGTCACCATATTCTTGGGAA | Novel | NAA |
| GGTGGAGCCCTCAGGCTCAG | Novel | NGC |
| GGTTGCGTCAGCAAACACTT | Novel | NGC |
| GTCACCATATTCTTGGGAAC | Novel | NAG |
| GTCCACCACGAGTCTAGACTC | EMSbeam16/ | NNNRRT |
| | MSPbeam2 | |
| GTCCATGCCCCAAAGCCACC | Novel | NAA |
| GTCCCGTCGGCGCTGAATCC | Novel | NGC |
| GTCGCAGAAGATCTCAATCT | Novel | NGG |
| GTCTGTGCCTTCTCATCTGC | Novel | NGG |
| GTGGACTTCTCTCAATTTTC | Novel | NAG |
| GTTCCGCAGTATGGATCGGC | EMSbeam17 | NGA |
| GTTCCGCAGTATGGATCGGC | Novel | NNAGGA |
| GTTTACTAGTGCCATTTGTT | Novel | NAG |
| GTTTACTAGTGCCATTTGTTC | Novel | NNNRRT |
| TAAAACGCCGCAGACACATC | Novel | NAG |
| TAAAACGCCGCAGACACATCC | EMSbeam18 | NNNRRT |
| TAGGCAGAGGTGAAAAAGTTG | Novel | NNNRRT |
| TCACCATATTCTTGGGAACA | Novel | NGA |
| TCAGTTTACTAGTGCCATTTG | Novel | NNNRRT |
| TCCATGCCCCAAAGCCACCC | Novel | NAG |
| TCCCCCTAGAAAATTGAGAG | Novel | NAG |
| TCCGCAGTATGGATCGGCAG | EMSbeam19 | NGG |
| TCCTCTGCCGATCCATACTG | EMSbeam20 | NGG |
| TCGCAGAAGATCTCAATCTC | Novel | NGG |
| TCTCAATCGCCGCGTCGCAG | Novel | NAG |
| TCTTCTGCGACGCGGCGATT | Novel | NAG |
| TCTTGTTCCCAAGAATATGG | Novel | NGA |
| TGAGATCTTCTGCGACGCGG | Novel | NGA |
| TGAGCCTGAGGGCTCCACCC | Novel | NAA |
| TGAGGCATAGCAGCAGGATG | Novel | NAG |
| TGGACTTCTCTCAATTTTCT | EMSbeam21 | NGG |
| TGGCCAAAATTCGCAGTCCC | Novel | NAA |
| TGGCTTTGGGGCATGGACAT | Novel | NGA |
| TGTGCACTTCGCTTCACCTC | Novel | NGC |
| TGTGCCTTGGGTGGCTTTGG | Novel | NGC |
| TTAGGCAGAGGTGAAAAAGT | Novel | NGC |
| TTCAAGCCTCCAAGCTGTGCC | EMSbeam22/ | NNGRRT |
| | MSPbeam9 | |
| TTCAAGCCTCCAAGCTGTGCC | EMSbeam22 | NNNRRT |
| TTCCCGAGATTGAGATCTTC | Novel | NGC |
| TTCCGCAGTATGGATCGGCA | Novel | NAG |
| TTCGCTTCACCTCTGCACGT | Novel | NGC |
| TTTGCTGACGCAACCCCCAC | EMSbeam23_whb | NGG |

The guide nucleic acids tested are described in Table 18A (see also FIG. 11).

TABLE 18A
Guide nucleic acids

| | | | | Areas |
|---|---|---|---|---|
| gRNA | Sequence | PAM | CAS | targeted |
| E1 | AAGGCACAGCTTGGAGGCTTG | NNNRRT | SaCas9 | Core |
| E2 | ACCAATTTATGCCTACAGCCT | NNNRRT | SaCas9 | X |
| E3 | AGGAGGCTGTAGGCATAAAT | NGG | SpCas9 | X |
| E4 | AGGAGTTCCGCAGTATGGAT | NGG | SpCas9 | Pol |
| E5 | CAAGCCTCCAAGCTGTGCCT | NGG | SpCas9 | Core |
| E6 | CAAGGCACAGCTTGGAGGCT | NGA | SpCas9 | Core |
| E7 | CCGCAGTATGGATCGGCAGA | NGA | SpCas9 | Pol |
| E8 | CCTCTGCCGATCCATACTGC | NGA | SpCas9 | Pol |
| E9 | CTAGACTCGTGGTGGACTTCT | NNNRRT | SaCas9 | Pol, S |
| E10 | GAACTCCCTCGCCTCGCAGAC | NNNRRT | SaCas9 | Pol, Core |
| E11 | GAAGAACTCCCTCGCCTCGC | NGA | SpCas9 | Pol, Core |
| E12 | GACTTCTCTCAATTTTCTAG | NGG | SpCas9 | Pol, S |
| E13 (M66) | GAGAAGTCCACCACGAGTCT | NGA | SpCas9 | Pol, S |
| E14 | GCTGCTATGCCTCATCTTCTT | NNNRRT | SaCas9 | Pol, S |
| E15 | GGACTTCTCTCAATTTTCTA | NGG | SpCas9 | Pol, S |
| E16 | GTCCACCACGAGTCTAGACTC | NNNRRT | SaCas9 | Pol, S |
| E17 | GTTCCGCAGTATGGATCGGC | NGA | SpCas9 | Pol |
| E18 | TAAAACGCCGCAGACACATCC | NNNRRT | SaCas9 | Pol, S |
| E19 | TCCGCAGTATGGATCGGCAG | NGG | SpCas9 | Pol |
| E20 | TCCTCTGCCGATCCATACTG | NGG | SpCas9 | Pol |
| E21 | TGGACTTCTCTCAATTTTCT | NGG | SpCas9 | Pol, S |
| E22 | TTCAAGCCTCCAAGCTGTGCC | NNGRRT | SaCas9 | Core |
| E22 | TTCAAGCCTCCAAGCTGTGCC | NNNRRT | SaCas9 | Core |
| E23 | TTTGCTGACGCAACCCCCAC | NGG | SpCas9 | Pol |
| E24 | TACTAACATTGAGGTTCCCG | NGA | SpCas9 | Pol, Core |

50,000 HBV-infected cells (pBtx693: cell line transduced with HBV sequence encoding X and Core HBV genes and pBtx536: cell line transduced with HBV sequences encoding Pol and S HBV genes) were plated one day prior to transfection with the guide RNA and a base editor (pBtx448 (BE4), pBtx543 (VRQR-BE4), or pBTx546 (sa-Cas9)). Cells were transfected with 250 ng of guide RNA and 750 ng of the construct encoding the base editor. Culture media was changed 1 and 3 days post-transfection. DNA was isolated from harvested cells, and edited regions were amplified by PCR using 10 μM primers. Each cell in the table identifies the primers used in the amplification reaction (e.g., 1058/ES86), the guide RNA (e.g., E1), and the base editor (e.g., 546). Conditions for the PCR reaction were as follows: an initial step of 98° C. for 30 seconds followed by 30 rounds of 98° C. for 10 seconds, 60° C. for 20 seconds, and 72° C. for 20 seconds, followed by a final step of 72° C. for 5 minutes.

The amplified products were subsequently sequenced to identify which nucleotides targeted by the guide RNA were edited (i.e., the specificity of the editing), as well as the efficiency of editing. Table 19 is a summary of the base editing efficiency and specificity for each of the guide RNAs listed in Table 20.

TABLE 19
Base editing efficiency and specificity

| | | | | | | | | Avg. |
|---|---|---|---|---|---|---|---|---|
| | Base Position Edited (Bold Rows) | | | | | | | Highest |
| gRNA | % Edit at each position (Rows below) | | | | | | | % Edit |
| E1 | 5 | 7 | 10 | | | | | 10.37 |
| | 0.24 | 17.23 | 4.1 | | | | | |
| | 0.31 | 3.5 | 0.63 | | | | | |
| E2 | 2 | 3 | 13 | 16 | | | | 24.18 |
| | 0 | 0 | 23.17 | 4.32 | | | | |
| | 2.49 | 4.12 | 33.23 | 5.29 | | | | |
| | 0.62 | 1.14 | 16.15 | 2.5 | | | | |
| E3 | 7 | | | | | | | 1.31 |
| | 2.62 | | | | | | | |
| | 0 | | | | | | | |
| E4 | | 8 | 9 | 11 | | | | 54.23 |
| | | 59 | 55.25 | 1.55 | | | | |
| | | 47.73 | 45.42 | 0.45 | | | | |
| | | 55.97 | 51.17 | 1.73 | | | | |
| E5 | 1 | 5 | 6 | 8 | 9 | | | 22.89 |
| | 1.35 | 2.17 | 10.25 | 11.75 | 9.78 | | | |
| | 9.53 | 7.19 | 29.63 | 35.37 | 28.98 | | | |
| | 2.64 | 11.46 | 28.78 | 32.25 | 28.56 | | | |
| E6 | | 6 | 8 | | | | | 36.04 |
| | | 39.98 | 35.13 | | | | | |
| | | 28.63 | 39.09 | | | | | |
| | | 21.92 | 33.91 | | | | | |
| E7 | | 1 | 2 | 4 | 14 | | | 51.29 |
| | | 46.08 | 34.59 | 8.9 | 28.72 | | | |
| | | 56.62 | 44.53 | 10.5 | 37.31 | | | |
| | | 51.17 | 41.05 | 9.7 | 33.93 | | | |
| E8 | | 4 | 7 | 8 | 12 | | | 44.72 |
| | | 44.54 | 13.45 | 13.85 | 13.88 | | | |
| | | 42.28 | 8.85 | 10.45 | 13.28 | | | |
| | | 47.35 | 14.53 | 17.45 | 17.51 | | | |
| E9 | | 1 | 6 | 8 | | | | 4.65 |
| | | 3.64 | 2.63 | 5.87 | | | | |
| | | 2.24 | 1.47 | 4.73 | | | | |
| | | 1.64 | 1.29 | 3.35 | | | | |
| E10 | 4 | 6 | 7 | 8 | 10 | 12 | 13 | 38.72 |
| | 1.01 | 29.15 | 19.25 | 18.69 | 42.17 | 2.12 | 9.14 | |
| | 1.25 | 24.05 | 15.7 | 15.43 | 31.85 | 2.71 | 8.28 | |
| | 1.36 | 29.67 | 19.88 | 20.05 | 42.15 | 2.06 | 8.01 | |
| E11 | | 7 | 9 | 10 | 11 | 13 | | 11.79 |
| | | 9.26 | 13.19 | 5.05 | 3.85 | 4.67 | | |
| | | 6.07 | 10.83 | 4.07 | 2.53 | 3.4 | | |
| | | 8.3 | 11.35 | 4.63 | 3.67 | 4.58 | | |
| E12 | | 3 | 6 | 8 | 10 | | | 43.91 |
| | | 0.33 | 32.61 | 36.17 | 8.22 | | | |
| | | 0.13 | 9.03 | 9.33 | 2.49 | | | |
| | | 0.17 | 11.97 | 86.22 | 2.82 | | | |
| E13 | | | | | | | | 19.47 |
| (M66) | | 8 | 9 | 11 | 12 | | | |
| | | 20.53 | 8.47 | 2.31 | 0.75 | | | |
| | | 26.03 | 9.21 | 1.99 | 0.54 | | | |
| | | 11.84 | 4.79 | 1.52 | 0.42 | | | |
| E14 | | 5 | 10 | 11 | 13 | 16 | | 7.54 |
| | | 0.01 | 0.33 | 2.24 | 5.16 | 4.92 | | |
| | | 0 | 0.67 | 3.17 | 7.04 | 6.61 | | |
| | | 0.01 | 1.3 | 4.84 | 10.41 | 10.3 | | |
| E15 | | 4 | 7 | 9 | 11 | | | 5 |
| | | 1.6 | 4.3 | 3.76 | 2.14 | | | |
| | | 1.79 | 4.57 | 3.52 | 2.2 | | | |
| | | 2.73 | 6.13 | 5.33 | 3.61 | | | |
| E16 | | 3 | 4 | 6 | 7 | 9 | 14 | 28.76 |
| | | 19.79 | 5.81 | 7.58 | 10.3 | 26.74 | 44.32 | |
| | | 20.55 | 7.4 | 7.12 | 7.99 | 25.89 | 38.01 | |
| | | 1.99 | 0.91 | 1.05 | 1.19 | 2.63 | 3.95 | |
| E17 | | 4 | 5 | 7 | 17 | | | |
| | | 57.01 | 55.99 | 13.37 | 1.97 | | | 57.71 |
| | | 55.63 | 51.62 | 12.52 | 4.73 | | | |
| | | 60.48 | 57.62 | 17.93 | 3.82 | | | |
| E18 | | 6 | 8 | 9 | 11 | | | |
| | | 1.68 | 0.52 | 1.52 | 0.34 | | | 10.22 |
| | | 16.99 | 5.23 | 15.96 | 4.49 | | | |
| | | 11.99 | 3.59 | 10.91 | 3.6 | | | |
| E19 | | 2 | 3 | 5 | | | | |
| | | 53.21 | 49.12 | 24.46 | | | | 44.63 |
| | | 38.64 | 35.38 | 18.57 | | | | |
| | | 42.03 | 38.94 | 23.8 | | | | |
| E20 | | 2 | 3 | 5 | 8 | 9 | 13 | |
| | | 5.05 | 5.88 | 52.37 | 19.33 | 19.03 | 16.47 | 50.74 |
| | | 4.83 | 5.64 | 50.42 | 18.55 | 21.86 | 21.38 | |
| | | 5.73 | 7.13 | 49.43 | 13.98 | 16.3 | 21.61 | |
| E21 | | 5 | 8 | 10 | 12 | | | 15.40 |
| | | 6.93 | 8.98 | 5.85 | 2.7 | | | |
| | | 16.16 | 20.1 | 13.3 | 5.95 | | | |
| | | 13.32 | 17.13 | 12.13 | 5.67 | | | |
| E22 | | 3 | 7 | 8 | 10 | 11 | | 0.01 |
| | | 0 | 0 | 0 | 0 | 0 | | |
| | | 0.02 | 0 | 0.01 | 0.01 | 0.02 | | |
| | | 0.02 | 0.01 | 0 | 0.02 | 0.01 | | |
| E23 | | 5 | 9 | | | | | 6.35 |
| | | 3.61 | 1.83 | | | | | |
| | | 8.62 | 3.59 | | | | | |
| | | 6.83 | 3.31 | | | | | |
| E24 | | 3 | 7 | 17 | | | | 47.51 |
| | | 37.98 | 54.1 | 6.99 | | | | |
| | | 34.43 | 49.5 | 6.77 | | | | |
| | | 24.76 | 38.92 | 7.49 | | | | |
| E95 | | 3 | 4 | 5 | 7 | 8 | | 45.29 |
| | | 3.44 | 11.92 | 14.63 | 16.61 | 16.01 | | |
| | | 7.71 | 33.79 | 46.19 | 56.45 | 52.91 | | |
| | | 15.11 | 48.31 | 62.82 | 63.65 | 66.96 | | |
| E96 | | 3 | 4 | 6 | 7 | 8 | | 0.01 |
| | | 0 | 0 | 0 | 0 | 0 | | |
| | | 0.02 | 0.01 | 0 | 0.01 | 0 | | |
| | | 0 | 0 | 0 | 0 | 0 | | |

Tables 19 and 20 identify substitutions in amino acids, and their frequencies, which would be the result of BE4 and ABE-mediated editing using guide RNAs designed to target super-conserved regions of the HBV genome.

TABLE 20
In silico analysis of the amino acid substitutions resulting from BE4 base editing with
a particular guide RNA selected for targeting conserved regions of HBV genome

| | | | Mutations | Amino acid | No. C |
|---|---|---|---|---|---|
| Guide | | | Introduced by BE4 | substitution | per |
| Sequence | PAM | Guide Id | base editors | Frequencies | window |
| GTTCCGCAGTATGGAT | NGA | E17 | Pol: A717T, | Pol: 717A = 99.79%, Pol: | 3 |
| CGGC | | | Pol: E718K | 717T = 0.02%, Pol: 718E = | |
| | | | | 99.81%, Pol: 718K = 0.05% | |
| AGGAGTTCCGCAGTAT | NGG | E4 | Pol: E718K | Pol: 718E = 99.81%, Pol: | 1 |
| GGAT | | | | 718K = 0.05% | |
| CCGCAGTATGGATCGG | NGA | E7 | Pol: A717T | Pol: 717A = 99.79%, Pol: | 1 |
| CAGA | | | | 717T = 0.02% | |
| TCCTCTGCCGATCCATA | NGG | E20 | Pol: P713S | Pol: 713P = 99.78%, Pol: | 2 |
| CTG | | | | 713S = 0.01% | |
| TACTAACATTGAGGTTC | NGA | E24_splice | Core: C183Y, | Core: 183C = 98.81%, | 1 |
| CCG | | (not | Pol:V48I | Core: 183Y = 0.02%, | |
| | | conserved) | | Pol: 48I = 0.01%, | |
| | | | | Pol: 48V = 99.47% | |
| CGCCCACCGAATGTTG | NGG | E95 (X- | X: H86Y, | X: 86H = 81.50%, | 4 |
| CCCA | | Stop, not | X: R87Stop | X: 86Y = 0.08%, | |
| | | conserved) | | X: 87* = 0.00%, | |
| | | | | X: 87R = 22.91% | |
| CCTCTGCCGATCCATAC | NGA | E8 | Pol: P713L | Pol: 713L = 0.06%, | 3 |
| TGC | | | | Pol: 713P = 99.78% | |
| TCCGCAGTATGGATCG | NGG | E19 | Pol: A717T | Pol: 717A = 99.79%, | 1 |
| GCAG | | | | Pol: 717T = 0.02% | |
| GACTTCTCTCAATTTTCT | NGG | E12 | S: S38F, S: L39F, | Pol: 382F = 0.05%, Pol: | 2 |
| AG | | | Pol: S382F | 382S = 99.86%, S: 38F = | |
| | | | | 0.01%, S: 38S = 99.93%, | |
| | | | | S: 39F = 0.04%, | |
| | | | | S: 39L = 99.84% | |
| GAACTCCCTCGCCTCGC | NNNRRT | E10 | Core: T160I, Core: | Core: 160I = 0.02%, Core: | 5 |
| AGAC | | | P161F, | 160T = 99.81%, Core: | |
| | | | Core: S162L, Pol: | 161F = 0.00%, Core: | |
| | | | L25F, | 161P = 99.82%, Core: | |
| | | | Pol: P26F, Pol: R27C | 162L = 0.03%, Core: 162S = | |
| | | | | 99.85%, Pol: 25F = 0.01%, | |
| | | | | Pol: 25L = 99.84%, Pol: | |
| | | | | 26F = 0.00%, Pol: | |
| | | | | 26P = 99.88%, Pol: 27C = | |
| | | | | 0.02%, Pol: 27 = 98.51% | |
| CAAGGCACAGCTTGGA | NGA | E6 | | | 2 |
| GGCT | | | | | |
| GTCCACCACGAGTCTA | NNNRRT | E16/2 | S: W35Stop, | Pol: 378I = 0.00%, Pol: | 5 |
| GACTC | | | S: W36Stop, Pol: | 378V = 99.80%, Pol: | |
| | | | V378I, Pol: V379I, | 379I = 0.00%, Pol: 379V = | |
| | | | Pol: D380N | 299.86%, Pol: 380D = | |
| | | | | 99.88%, Pol: 380N = 0.01%, | |
| | | | | S: 35* = 0.00%, S: 35W = | |
| | | | | 99.77%, S: 36* = 0.00%, | |
| | | | | S:36W = 99.55% | |
| ACCAATTTATGCCTACA | NNNRRT | E2 | | | 1 |
| GCCT | | | | | |
| CAAGCCTCCAAGCTGT | NGG | E5 | | | 3 |
| GCCT | | | | | |
| GAGAAGTCCACCACGA | NGA | M66/E13 | S: W36Stop, | Pol: 380D = 99.88%, Pol: | 1 |
| GTCT | | | Pol: D380N | 380N = 0.01%, S: 36* = | |
| | | | | 0.00%, S: 36W = 99.55% | |
| TGGACTTCTCTCAATTT | NGG | E21 | S: T37I, S: S38F | S: 37I = 0.02%, S: 37T = | 2 |
| TCT | | | | 99.87%, S: 38F = 0.01%, | |
| | | | | S: 38S = 99.93% | |
| GAAGAACTCCCTCGCCT | NGA | E11 | Core: T160I, | Core: 160I = 20.02%, | 1 |
| CGC | | | Pol: L25F | Core: 160T = 99.81%, | |
| | | | | Pol: 25F = 0.01%, Pol: | |
| | | | | 25L = 99.84% | |
| AAGGCACAGCTTGGAG | NNNRRT | E1 | | | 3 |
| GCTTG | | | | | |
| TAAAACGCCGCAGACA | NNNRRT | E18 | S: R78Q, S: R79H, | Pol: 422A = 99.91%, Pol: | 3 |
| | | | Pol: A422T | 422T = 0.01%, S: 78Q = | |
| CATCC | | | | 0.03%, S: 78R = 99.91%, | |
| | | | | S: 79H = 0.36%, S: 79R = | |
| | | | | 99.38% | |
| GCTGCTATGCCTCATCT | NNNRRT | E14 | Pol: A432V, Pol: | Pol: 432A = 99.51%, Pol: | 2 |
| TCTT | | | P434S | 432V = 0.04%, Pol: 434P = | |
| | | | | 99.89%, Pol: 434S = 0.06% | |
| TTTGCTGACGCAACCCC | NGG | E23 | Pol: A688V | Pol: 688A = 99.75%, Pol: | 1 |
| CAC | | | | 688V = 0.01% | |
| GGACTTCTCTCAATTTT | NGG | E15 | S: T37I, S: S38F | S: 37I = 0.02%, S: 37T = | 2 |
| CTA | | | | 99.87%, S: 38F = 0.01%, | |
| | | | | S: 38S = 99.93% | |
| CTAGACTCGTGGTGGA | NNNRRT | E9 | S: S34L, Pol: L377F | Pol: 377F = 0.01%, Pol: | 2 |
| CTTCT | | | | 377L = 99.91%, S: 34L = | |
| | | | | 0.41%, S: 34S = 99.40% | |
| AGGAGGCTGTAGGCAT | NGG | E3 | | | 1 |
| AAAT | | | | | |
| TTCAAGCCTCCAAGCTG | NNGRRT | E22/9 | | | 4 |
| TGCC | | | | | |
| ACTCCTCCCAGTCTTTA | NNNRRT | E96 | X: W120Stop, | X: 120* = 0.00%, | 5 |
| AACA | | | X: E121K, | X: 120W = 99.79%, | |
| | | | X: E122K | X: 121E = 99.82%, | |
| | | | | X: 121K = 0.04%, | |
| | | | | X: 122E = 99.13%, | |
| | | | | X: 122K = 0.05% | |

TABLE 21
In silico analysis of the amino acid substitutions resulting from ABE base editing with a particular guide RNA
selected for targeting conserved regions of HBV genome

| | Mutations | | Minimum | |
|---|---|---|---|---|
| | Introduced by ABE | | allele | |
| Guide ID | base editors | Allele Frequencies | frequency | No. A per window |
| E17 | | | 100 | 1 |
| E4 | Pol: L719P | Pol: 719L = 99.80%, Pol: 719P = | 0.082362631 | 1 |
| | | 0.08% | | |
| E7 | | | 100 | 2 |
| E20 | | | 100 | 0 |
| E24_splice (not | Core: C183R, Core: | Core: 183C = 98.81%, Core: 183R = | 0.073421439 | 3 |
| conserved) | Stop1840., Pol: V48A | 0.07%, Pol: 48A = 0.22%, Pol: 48V = | | |
| | | 99.47% | | |
| E95 (X-Stop, not | X: H86R | X: 86H = 81.50%, X: 86R = 11.10% | 11.09879032 | 1 |
| conserved) | | | | |
| E8 | | | 100 | 0 |
| E19 | | | 100 | 1 |
| E12 | | | 100 | 0 |
| E10 | Core: T160A | Core: 160A = 0.04%, Core: 160T = | 0.03671072 | 1 |
| | | 99.81% | | |
| E6 | | | 100 | 1 |
| E16/2 | S: W35R, S: W36R, Pol: | Pol: 378A = 0.09%, Pol: 378V = 9 | 0.070596541 | 2 |
| | V378A, Pol: V379A | 9.80%, Pol: 379A = 0.07%, Pol: | | |
| | | 379V = 99.86%, S: 35R = 0.07%, | | |
| | | S: 35W = 99.77%, S: 36R = 0.10%, | | |
| | | S: 36W = 99.55% | | |
| E2 | X: L141P | X: 141L = 99.53%, X: 141P = 0.01% | 0.010080645 | 3 |
| E5 | | | 100 | 0 |
| M66/E13 | S: S38P, Pol: F381P | Pol: 381F = 99.93%, Pol: 381P = | 0 | 2 |
| | | 0.00%, S: 38P = 0.04%, S: 38S = 9 | | |
| | | 9.93% | | |
| E21 | S: 137A, Pol: D380G | Pol: 380D = 99.88%, Pol: 380G = | 0.03529827 | 1 |
| | | 0.04%, S: 37A = 0.06%, S: 371 = | | |
| | | 99.87% | | |
| E11 | Core: T160A, Pol: E24G | Core: 160A = 0.04%, Core: 160T = | 0.03671072 | 2 |
| | | 99.81%, Pol: 24E = 99.75%, | | |
| | | Pol: 24G = 0.11% | | |
| E1 | | | 100 | 2 |
| E18 | S: F80P, Pol: F423P | Pol: 423F = 99.84%, Pol: 423P = | 0 | 3 |
| | | 0.00%, S: 80F = 99.26%, S: 80P = | | |
| | | 0.00% | | |
| E14 | Pol: M433V | Pol: 433M = 99.88%, Pol: 433V = | 0.02353218 | 1 |
| | | 0.02% | | |
| E23_whb | Pol: D689G | Pol: 689D = 99.87%, Pol: 689G = | 0.02353218 | 1 |
| | | 0.02% | | |
| E15 | | | 100 | 0 |
| E9 | S: D33G, Pol: R376G | Pol: 376G = 0.02%, Pol: 376R = | 0.02353218 | 2 |
| | | 99.85%, S: 33D = 99.75%, S: 33G = | | |
| | | 0.08% | | |
| E3 | | | 100 | 1 |
| E22/9 | | | 100 | 2 |
| E96 | X: W120R | X: 120R = 0.13%, X: 120W = 99.79% | 0.131048387 | 1 |
| | | | 100 | 2 |

### Example 4: Additional gRNAs were Contemplated for Targeting HBV

Table 22 provides a list of sequences targeted by guide RNAs designed to introduce missense mutations in the HBV genome using an ABE base editor. The sequences targeted by the guide RNAs were at least 50% conserved between HBV genotypes A and D. Amino acid substitutions which would result from application of gRNA and ABE were analyzed in silico, and we further selected those gRNAs that would generate amino acid substitution that occur in less than 0.05% of known sequenced HBV genes. That would imply that the base editing with the selected gRNA would lead to a misfunctional HBV protein.

TABLE 22
ABE gRNA Functional List

| guide_seq | guide_id | pam |
|---|---|---|
| AAAAACCCCGCCTGTAACAC | Novel | NAG |
| AAAAAGTTGCATGGTGCTGG | Novel | NGC |
| AAAACAAGCGGCTAGGAGTTC | Novel | NNNRRT |
| AAAACGCCGCAGACACATCC | Novel | NGC |
| AAACAAGCGGCTAGGAGTTC | Novel | NGC |
| AAAGAATTTGGAGCTACTGT | Novel | NGA |
| AAAGCCAAACAGTGGGGGAA | Novel | NGC |
| AAATTGAGAGAAGTCCACCA | Novel | NGA |
| AACAAATGGCACTAGTAAAC | Novel | NGA |
| AACAAGAAAAACCCCGCCTG | Novel | NAA |
| AACAGTGGGGGAAAGCCCTA | Novel | NGA |
| AACATCACATCAGGATTCCT | Novel | NGG |
| AACATGGAGAACATCACATC | Novel | NGG |
| AACCACTGAACAAATGGCAC | Novel | NAG |
| AACCCCCACTGGCTGGGGCT | Novel | NGG |
| AAGAAGATGAGGCATAGCAG | Novel | NAG |
| AAGAAGTCAGAAGGCAAAAA | Novel | NGA |
| AAGAAGTCAGAAGGCAAAAAC | Novel | NNGRRT |
| AAGAAGTCAGAAGGCAAAAAC | Novel | NNNRRT |
| AAGAATCCTCACAATACCGC | Novel | NGA |
| AAGAATTTGGAGCTACTGTG | Novel | NAG |
| AAGGAAAGAAGTCAGAAGGC | Novel | NAA |
| AATGTCAACGACCGACCTTG | Novel | NGG |
| AATTGAGAGAAGTCCACCAC | Novel | NAG |
| ACAAATGGCACTAGTAAACT | Novel | NAG |
| ACAAGAATCCTCACAATACCG | Novel | NNGRRT |
| ACAAGAATCCTCACAATACCG | Novel | NNNRRT |
| ACACATCCAGCGATAACCAGG | M3 | NNNRRT |
| ACAGGAGGTTGGTGAGTGAT | Novel | NGG |
| ACAGGAGGTTGGTGAGTGATT | Novel | NNNRRT |
| ACAGTGGGGGAAAGCCCTAC | Novel | NNACCA |
| ACATGGAGAACATCACATCA | Novel | NGA |
| ACATTTAAACCCTAACAAAA | Novel | NAA |
| ACCAATTTATGCCTACAGCCT | E2 | NNNRRT |
| ACGAACCACTGAACAAATGGC | M5 | NNNRRT |
| ACGCAACCCCCACTGGCTGG | Novel | NGC |
| ACTGAACAAATGGCACTAGT | Novel | NAA |
| AGAAAGGCCTTGTAAGTTGG | Novel | NGA |
| AGAACATCACATCAGGATTC | Novel | NNAGGA |
| AGAAGAACTCCCTCGCCTCG | Novel | NAG |
| AGAAGATGAGGCATAGCAGC | Novel | NGG |
| AGAAGGGGACGAGAGAGTCC | Novel | NAA |
| AGACAAAAGAAAATTGGTAA | Novel | NAG |
| AGACAAAAGAAAATTGGTAAC | Novel | NNNRRT |
| AGACACATCCAGCGATAACC | Novel | NGG |
| AGACGGAGAAGGGGACGAGA | Novel | NAG |
| AGAGAAGTCCACCACGAGTC | Novel | NAG |
| AGAGAGGTGCGCCCCGTGGT | Novel | NGG |
| AGATGAGAAGGCACAGACGG | Novel | NGA |
| AGCGCCGACGGGACGTAAAC | Novel | NAA |
| AGCGCCGACGGGACGTAAAC | Novel | NNAGGA |
| AGCTCCAAATTCTTTATAAGG | Novel | NNNRRT |
| AGGAAAGAAGTCAGAAGGCA | Novel | NAA |
| AGGAGGTTGGTGAGTGATTG | Novel | NAG |
| AGGCAAAAACGAGAGTAACTC | Novel | NNNRRT |
| AGGCATAGCAGCAGGATGAA | Novel | NAG |
| AGGGTTTAAATGTATACCCA | Novel | NAG |
| AGGTATGTTGCCCGTTTGTCC | Novel | NNNRRT |
| AGTAACTCCACAGTAGCTCC | Novel | NAA |
| AGTCCAAGGAATACTAACAT | M78 | NGA |
| AGTCCCCAACCTCCAATCAC | Novel | NNACCA |
| AGTGATTGGAGGTTGGGGACT | Novel | NNGRRT |
| AGTGATTGGAGGTTGGGGACT | Novel | NNNRRT |
| AGTGCGAATCCACACTCCGA | Novel | NAG |
| AGTGTGGATTCGCACTCCTC | Novel | NAG |
| ATAAAGAATTTGGAGCTACTG | Novel | NNGRRT |
| ATAAAGAATTTGGAGCTACTG | Novel | NNNRRT |
| ATAAATTGGTCTGCGCACCA | Novel | NNACCA |
| ATATGGATGATGTGGTATTG | Novel | NGG |
| ATCCATACTGCGGAACTCCT | Novel | NGC |
| ATGAGGCATAGCAGCAGGAT | Novel | NAA |
| ATGATGTGGTATTGGGGGCC | Novel | NAG |
| ATGGATGATGTGGTATTGGG | Novel | NGC |
| ATGGGATGGGAATACAGGTG | Novel | NAA |
| ATGTCAACGACCGACCTTGA | Novel | NGC |
| ATTCAGCGCCGACGGGACGT | Novel | NAA |
| ATTGTGAGGATTCTTGTCAA | Novel | NAA |
| ATTTAAACCCTAACAAAACAA | Novel | NNNRRT |
| CAAAAACGAGAGTAACTCCA | Novel | NAG |
| CAAAAGAAAATTGGTAACAG | Novel | NGG |
| CAAAATTCGCAGTCCCCAACC | Novel | NNNRRT |
| CAAATGGCACTAGTAAACTG | Novel | NGC |
| CAAGAATCCTCACAATACCG | Novel | NAG |
| CAATACCGCAGAGTCTAGACT | M1 | NNNRRT |
| CACCACGAGTCTAGACTCTG | M36 | NGG |
| CACTGAACAAATGGCACTAG | Novel | NAA |
| CAGACACATCCAGCGATAAC | Novel | NAG |
| CAGACGGAGAAGGGGACGAG | Novel | NGA |
| CAGGAGGTTGGTGAGTGATT | Novel | NGA |
| CATAAATTGGTCTGCGCACC | Novel | NGC |
| CATACTGCGGAACTCCTAGC | Novel | NGC |
| CATTTAAACCCTAACAAAAC | Novel | NAA |
| CCCAACCTCCAATCACTCAC | Novel | NAA |
| CCCGAGATTGAGATCTTCTG | Novel | NGA |
| CCCTAGAAGAAGAACTCCCT | Novel | NGC |
| CCTACGAACCACTGAACAAA | Novel | NGG |
| CCTAGAAAATTGAGAGAAGT | Novel | NNACCA |
| CCTCACAATACCGCAGAGTC | Novel | NAG |
| CCTGAACTGGAGCCACCAGC | Novel | NGG |
| CGAATTTTGGCCAAGACACA | Novel | NGG |
| CGAGCAAAACAAGCGGCTAG | Novel | NAG |
| CGCAGAAGATCTCAATCTCG | Novel | NGA |
| CGCAGACCAATTTATGCCTA | Novel | NAG |
| CGCAGAGTCTAGACTCGTGG | M35 | NGG |
| CGCCGACGGGACGTAAACAA | Novel | NGG |
| CGCGTAAAGAGAGGTGCGCCC | Novel | NNNRRT |
| CGTGCAGAGGTGAAGCGAAG | Novel | NGC |
| CTACGAACCACTGAACAAAT | Novel | NGC |
| CTAGACTCGTGGTGGACTTCT | E9 | NNNRRT |
| CTCAATCGCCGCGTCGCAGA | M77 | NGA |
| CTCACAATACCGCAGAGTCT | Novel | NGA |
| CTCACCAACCTCCTGTCCTC | Novel | NAA |
| CTGAAAGCCAAACAGTGGGG | Novel | NAA |
| CTGAACTGGAGCCACCAGCAG | Novel | NNNRRT |
| CTGACGCAACCCCCACTGGC | Novel | NGG |
| CTGCGAGCAAAACAAGCGGCT | Novel | NNGRRT |
| CTGCGAGCAAAACAAGCGGCT | Novel | NNNRRT |
| CTGTGCCAAGTGTTTGCTGA | Novel | NGC |
| CTTGTAAGTTGGCGAGAAAG | Novel | NGA |
| CTTGTCAACAAGAAAAACCC | Novel | NGC |
| CTTGTTGACAAGAATCCTCA | Novel | NAA |
| GAAAAACCCCGCCTGTAACA | Novel | NGA |
| GAAAATTGAGAGAAGTCCACC | Novel | NNGRRT |
| GAAAATTGAGAGAAGTCCACC | Novel | NNNRRT |
| GAAAATTGGTAACAGCGGTA | Novel | NAA |
| GAAAGCCAAACAGTGGGGGA | Novel | NAG |
| GAAAGCCCTACGAACCACTGA | Novel | NNNRRT |
| GAACATCACATCAGGATTCC | Novel | NAG |
| GAACATGGAGAACATCACAT | Novel | NAG |
| GAACTCCCTCGCCTCGCAGAC | E10 | NNNRRT |
| GAAGAACTCCCTCGCCTCGC | E11 | NGA |
| GAAGAAGAACTCCCTCGCCT | Novel | NGC |
| GAAGATGAGGCATAGCAGCA | Novel | NGA |
| GAAGGAAAGAAGTCAGAAGG | Novel | NAA |
| GACAAAAGAAAATTGGTAAC | Novel | NGC |
| GACAAACGGGCAACATACCT | Novel | NGA |
| GACAAACGGGCAACATACCTT | Novel | NNNRRT |
| GACAAGAATCCTCACAATAC | Novel | NGC |
| GACACATCCAGCGATAACCA | M67 | NGA |
| GACGCAACCCCCACTGGCTG | Novel | NGG |
| GACGTAAACAAAGGACGTCC | Novel | NGC |
| GAGAAGTCCACCACGAGTCT | M66 | NGA |
| GAGAGTAACTCCACAGTAGCT | Novel | NNNRRT |
| GAGGACAAACGGGCAACATAC | Novel | NNNRRT |
| GAGGACAGGAGGTTGGTGAG | Novel | NGA |
| GAGGCATAGCAGCAGGATGA | Novel | NGA |
| GAGGCATAGCAGCAGGATGA | Novel | NNAGGA |
| GAGGTGAAAAAGTTGCATGG | Novel | NGC |
| GAGGTGAAAAAGTTGCATGGT | Novel | NNNRRT |
| GAGGTGAAGCGAAGTGCACA | Novel | NGG |
| GAGTAACTCCACAGTAGCTC | Novel | NAA |
| GAGTCCAAGGAATACTAACAT | Novel | NNNRRT |
| GATCCATACTGCGGAACTCC | Novel | NAG |
| GATGAGAAGGCACAGACGGG | Novel | NAG |
| GATGAGGCATAGCAGCAGGA | Novel | NGA |
| GATGATGTGGTATTGGGGGC | Novel | NAA |
| GATTCAGCGCCGACGGGACG | Novel | NAA |
| GATTGGAGGTTGGGGACTGC | Novel | NAA |
| GCAAACACTTGGCACAGACC | Novel | NGG |
| GCAACTTTTTCACCTCTGCC | Novel | NAA |
| GCAGAAGATCTCAATCTCGG | Novel | NAA |
| GCAGACACATCCAGCGATAA | Novel | NNAGGA |
| GCAGACCAATTTATGCCTAC | Novel | NGC |
| GCAGAGTCTAGACTCGTGGT | M65 | NGA |
| GCATAAATTGGTCTGCGCAC | Novel | NAG |
| GCCCAAGGTCTTACATAAGA | Novel | NGA |
| GCCGACGGGACGTAAACAAA | Novel | NGA |
| GCCGCAGACACATCCAGCGA | Novel | NAA |
| GCCGCAGACACATCCAGCGA | Novel | NNACCA |
| GCGAATCCACACTCCGAAAG | Novel | NNACCA |
| GCGAGCAAAACAAGCGGCTA | Novel | NGA |
| GCGCCGACGGGACGTAAACA | Novel | NAG |
| GCTGCTATGCCTCATCTTCTT | E14 | NNNRRT |
| GGAAAGAAGTCAGAAGGCAA | Novel | NAA |
| GGAAAGCCCTACGAACCACT | Novel | NAA |
| GGCACTAGTAAACTGAGCCA | Novel | NGA |
| GGCAGACGGAGAAGGGGACGA | Novel | NNGRRT |
| GGCAGACGGAGAAGGGGACGA | Novel | NNNRRT |
| GGCATAGCAGCAGGATGAAG | Novel | NGG |
| GGCGTTCACGGTGGTCTCCA | Novel | NGC |
| GGGAAAGCCCTACGAACCAC | Novel | NGA |
| GGGACTGCGAATTTTGGCCA | Novel | NGA |
| GGGGACTGCGAATTTTGGCC | Novel | NAG |
| GGGGCATTTGGTGGTCTATA | Novel | NGC |
| GGGGCGCACCTCTCTTTACG | Novel | NGG |
| GGTATACATTTAAACCCTAA | Novel | NAA |
| GGTGAGTGATTGGAGGTTGG | Novel | NGA |
| GTAAACAAAGGACGTCCCGC | Novel | NNAGGA |
| GTAAACAAAGGACGTCCCGCG | Novel | NNGRRT |
| GTAAACAAAGGACGTCCCGCG | Novel | NNNRRT |
| GTAGGCATAAATTGGTCTGC | Novel | NNACCA |
| GTATACATTTAAACCCTAAC | Novel | NAA |
| GTCCAAGGAATACTAACATT | Novel | NAG |
| GTCCATGCCCCAAAGCCACC | Novel | NAA |
| GTCGCAGAAGATCTCAATCT | Novel | NGG |
| GTCTAGACTCTGCGGTATTG | Novel | NGA |
| GTCTAGACTCTGCGGTATTG | Novel | NNAGGA |
| GTCTAGACTCTGCGGTATTGT | Novel | NNGRRT |
| GTCTAGACTCTGCGGTATTGT | Novel | NNNRRT |
| GTGAAAAAGTTGCATGGTGC | Novel | NGG |
| GTGAGGATTCTTGTCAACAA | Novel | NAA |
| GTGCCAAGTGTTTGCTGACG | Novel | NAA |
| GTGCGAATCCACACTCCGAA | Novel | NGA |
| GTGGACTTCTCTCAATTTTC | Novel | NAG |
| GTGTGGATTCGCACTCCTCC | Novel | NGC |
| GTTAATCATTACTTCCAAAC | Novel | NAG |
| GTTACCAATTTTCTTTTGTCT | Novel | NNGRRT |
| GTTACCAATTTTCTTTTGTCT | Novel | NNNRRT |
| GTTAGTATTCCTTGGACTCA | Novel | NAA |
| GTTGGTGAGTGATTGGAGGT | Novel | NGG |
| GTTTACGTCCCGTCGGCGCT | Novel | NAA |
| GTTTACTAGTGCCATTTGTT | Novel | NAG |
| GTTTACTAGTGCCATTTGTTC | Novel | NNNRRT |
| TAAAACGCCGCAGACACATC | Novel | NAG |
| TAAAACGCCGCAGACACATCC | E18 | NNNRRT |
| TAAACAAAGGACGTCCCGCG | Novel | NAG |
| TAAAGAATTTGGAGCTACTG | Novel | NGG |
| TACTAGTGCCATTTGTTCAG | Novel | NGG |
| TAGCAGCAGGATGAAGAGGAA | Novel | NNNRRT |
| TATACATTTAAACCCTAACA | Novel | NAA |
| TATATGGATGATGTGGTATT | Novel | NGG |
| TATGGATGATGTGGTATTGG | M94 | NGG |
| TCACCATATTCTTGGGAACA | Novel | NGA |
| TCAGTTTACTAGTGCCATTTG | Novel | NNNRRT |
| TCCATGCCCCAAAGCCACCC | Novel | NAG |
| TCCTGAACTGGAGCCACCAG | Novel | NAG |
| TCGCAGAAGATCTCAATCTC | Novel | NGG |
| TCTAGACTCTGCGGTATTGT | Novel | NAG |
| TCTCAATCGCCGCGTCGCAG | Novel | NAG |
| TGAAAGCCAAACAGTGGGGG | Novel | NAA |
| TGACGCAACCCCCACTGGCT | Novel | NGG |
| TGAGGCATAGCAGCAGGATG | Novel | NAG |
| TGATTGGAGGTTGGGGACTG | Novel | NGA |
| TGCCCAAGGTCTTACATAAG | Novel | NGG |
| TGGACTTCTCTCAATTTTCT | E21 | NGG |
| TGGATGATGTGGTATTGGGGG | Novel | NNNRRT |
| TGGCCAAAATTCGCAGTCCC | Novel | NAA |
| TGGGGACTGCGAATTTTGGC | Novel | NAA |
| TGGTGAGTGATTGGAGGTTG | Novel | NGG |
| TGTAAGTTGGCGAGAAAGTG | Novel | NAA |
| TGTAGGCATAAATTGGTCTG | Novel | NGC |
| TGTGAGGATTCTTGTCAACA | Novel | NGA |
| TGTGCACTTCGCTTCACCTC | Novel | NGC |
| TGTGGAGTTACTCTCGTTTT | Novel | NGC |
| TGTTAGTATTCCTTGGACTCA | Novel | NNNRRT |
| TGTTTACGTCCCGTCGGCGC | Novel | NGA |
| TTAAACCCTAACAAAACAAA | Novel | NAG |
| TTCCCCCACTGTTTGGCTTT | Novel | NAG |
| TTCCCGAGATTGAGATCTTC | Novel | NGC |
| TTGCTGACGCAACCCCCACT | Novel | NGC |
| TTGGAGGACAGGAGGTTGGTG | Novel | NNNRRT |
| TTGGTGAGTGATTGGAGGTT | Novel | NGG |
| TTGTAAGTTGGCGAGAAAGT | Novel | NAA |
| TTGTGAGGATTCTTGTCAAC | Novel | NAG |
| TTTGCTGACGCAACCCCCAC | E23_whb | NGG |
| TTTGTTTACGTCCCGTCGGCG | Novel | NNGRRT |
| TTTGTTTACGTCCCGTCGGCG | Novel | NNNRRT |

Table 23 provides a list of guide RNAs that introduce missense mutations in the HBV genome using a BE4 base editor. The sequences of the guide RNAs were at least 5000 conserved between HBV genotypes A and D. Amino acid substitutions which would result from application of gRNA and BE4 were analyzed in silico, and we further selected those gRNAs that would generate amino acid substitution that occur in less than 0.05% of known sequenced HBV genes. That would imply that the base editing with the selected gRNA would lead to a misfunctional HBV protein.

TABLE 23
BE4 gRNA Functional list

| guide_seq | guide id | pam |
|---|---|---|
| AAAAACCCCGCCTGTAACAC | Novel | NAG |
| AAAACAAGCGGCTAGGAGTTC | Novel | NNNRRT |
| AAAACGCCGCAGACACATCC | Novel | NGC |
| AAACAAGCGGCTAGGAGTTC | Novel | NGC |
| AAAGCCAAACAGTGGGGGAA | Novel | NGC |
| AACCACTGAACAAATGGCAC | Novel | NAG |
| AACCCCCACTGGCTGGGGCT | Novel | NGG |
| AACCCCGCCTGTAACACGAG | Novel | NAG |
| AACGCCGCAGACACATCCAG | Novel | NGA |
| AAGAAGTCAGAAGGCAAAAA | Novel | NGA |
| AAGAAGTCAGAAGGCAAAAAC | Novel | NNGRRT |
| AAGAAGTCAGAAGGCAAAAAC | Novel | NNNRRT |
| AAGAATCCTCACAATACCGC | Novel | NGA |
| AAGCCCCAGCCAGTGGGGGT | Novel | NGC |
| AAGCCCTACGAACCACTGAA | Novel | NAA |
| AAGGGGACGAGAGAGTCCCA | Novel | NGC |
| AAGTCAGAAGGCAAAAACGA | Novel | NAG |
| AATCGCCGCGTCGCAGAAGAT | Novel | NNNRRT |
| AATTCGCAGTCCCCAACCTC | Novel | NAA |
| ACAAGAATCCTCACAATACCG | Novel | NNGRRT |
| ACAAGAATCCTCACAATACCG | Novel | NNNRRT |
| ACAAGCGGCTAGGAGTTCCG | Novel | NAG |
| ACACATCCAGCGATAACCAGG | M3 | NNNRRT |
| ACAGGCGGGGTTTTTCTTGT | M64 | NGA |
| ACATCCAGCGATAACCAGGA | Novel | NAA |
| ACGAACCACTGAACAAATGGC | M5 | NNNRRT |
| ACGCAACCCCCACTGGCTGG | Novel | NGC |
| ACGGGGCGCACCTCTCTTTA | Novel | NGC |
| ACTCCCTCGCCTCGCAGACG | Novel | NAG |
| ACTGAACAAATGGCACTAGT | Novel | NAA |
| ACTTCTCTCAATTTTCTAGG | Novel | NGG |
| ACTTTCTCGCCAACTTACAA | Novel | NGC |
| AGAAGAACTCCCTCGCCTCG | Novel | NAG |
| AGAAGTCAGAAGGCAAAAAC | Novel | NAG |
| AGAATCCTCACAATACCGCA | Novel | NAG |
| AGACACATCCAGCGATAACC | Novel | NGG |
| AGCCCTACGAACCACTGAAC | Novel | NAA |
| AGCGCCGACGGGACGTAAAC | Novel | NAA |
| AGCGCCGACGGGACGTAAAC | Novel | NNAGGA |
| AGCGGCTAGGAGTTCCGCAGT | Novel | NNGRRT |
| AGCGGCTAGGAGTTCCGCAGT | Novel | NNNRRT |
| AGCTCCAAATTCTTTATAAGG | Novel | NNNRRT |
| AGGAGTTCCGCAGTATGGAT | E4 | NGG |
| AGGCATAGCAGCAGGATGAA | Novel | NAG |
| AGGGCTTTCCCCCACTGTTT | Novel | NGC |
| AGTAACTCCACAGTAGCTCC | Novel | NAA |
| AGTAGCTCCAAATTCTTTAT | Novel | NAG |
| AGTCCAAGAGTCCTCTTATG | Novel | NAA |
| AGTCCAAGGAATACTAACAT | M78 | NGA |
| AGTCCCCAACCTCCAATCAC | Novel | NNACCA |
| AGTTCCGCAGTATGGATCGG | Novel | NAG |
| ATCCATACTGCGGAACTCCT | Novel | NGC |
| ATGAGGCATAGCAGCAGGAT | Novel | NAA |
| ATTCCTTGGACTCATAAGGT | Novel | NGG |
| ATTTAAACCCTAACAAAACAA | Novel | NNNRRT |
| ATTTGTTCAGTGGTTCGTAG | Novel | NGC |
| CAAAATTCGCAGTCCCCAACC | Novel | NNNRRT |
| CAAGAATCCTCACAATACCG | Novel | NAG |
| CAATACCGCAGAGTCTAGACT | M1 | NNNRRT |
| CAATGCTCAGGAGACTCTAA | Novel | NGC |
| CACCACGAGTCTAGACTCTG | M36 | NGG |
| CACTGAACAAATGGCACTAG | Novel | NAA |
| CAGACACATCCAGCGATAAC | Novel | NAG |
| CAGCCAGTGGGGGTTGCGTC | Novel | NGC |
| CAGCGCCGACGGGACGTAAA | Novel | NAA |
| CAGTAGCTCCAAATTCTTTA | Novel | NAA |
| CAGTAGCTCCAAATTCTTTAT | Novel | NNGRRT |
| CAGTAGCTCCAAATTCTTTAT | Novel | NNNRRT |
| CATAGCAGCAGGATGAAGAG | Novel | NAA |
| CCAGCCAGTGGGGGTTGCGT | Novel | NAG |
| CCGCAGTATGGATCGGCAGA | E7 | NGA |
| CCGTGTGTCTTGGCCAAAATT | Novel | NNNRRT |
| CCTACGAACCACTGAACAAA | Novel | NGG |
| CCTCACAATACCGCAGAGTC | Novel | NAG |
| CCTTGGACTCATAAGGTGGG | Novel | NAA |
| CGAGCAAAACAAGCGGCTAG | Novel | NAG |
| CGCAGACCAATTTATGCCTA | Novel | NAG |
| CGCCGCGTCGCAGAAGATCT | Novel | NAA |
| CGCGTAAAGAGAGGTGCGCCC | Novel | NNNRRT |
| CGGCTAGGAGTTCCGCAGTA | Novel | NGG |
| CGTCAGCAAACACTTGGCAC | Novel | NGA |
| CGTGTGTCTTGGCCAAAATT | Novel | NGC |
| CTACGAACCACTGAACAAAT | Novel | NGC |
| CTAGACTCGTGGTGGACTTCT | E9 | NNNRRT |
| CTAGCCGCTTGTTTTGCTCG | Novel | NAG |
| CTCACCAACCTCCTGTCCTC | Novel | NAA |
| CTCTCGTCCCCTTCTCCGTC | Novel | NGC |
| CTGAAAGCCAAACAGTGGGG | Novel | NAA |
| CTGACGCAACCCCCACTGGC | Novel | NGG |
| CTGCGAGCAAAACAAGCGGC | Novel | NAG |
| CTGCGAGCAAAACAAGCGGCT | Novel | NNGRRT |
| CTGCGAGCAAAACAAGCGGCT | Novel | NNNRRT |
| CTGTGCCAAGTGTTTGCTGA | Novel | NGC |
| CTTCTCTCAATTTTCTAGGG | M87 | NGA |
| CTTCTGCGACGCGGCGATTG | Novel | NGA |
| GAAAAACCCCGCCTGTAACA | Novel | NGA |
| GAAAGCCAAACAGTGGGGGA | Novel | NAG |
| GAAAGCCCTACGAACCACTGA | Novel | NNNRRT |
| GAACTCCCTCGCCTCGCAGAC | E10 | NNNRRT |
| GAACTCCTAGCCGCTTGTTT | Novel | NGC |
| GAAGAACTCCCTCGCCTCGC | E11 | NGA |
| GAAGTCAGAAGGCAAAAACG | Novel | NGA |
| GACAAACGGGCAACATACCT | Novel | NGA |
| GACAAACGGGCAACATACCTT | Novel | NNNRRT |
| GACACATCCAGCGATAACCA | M67 | NGA |
| GACGCAACCCCCACTGGCTG | Novel | NGG |
| GACTTCTCTCAATTTTCTAG | E12 | NGG |
| GAGAAGTCCACCACGAGTCT | M66 | NGA |
| GAGCCTGAGGGCTCCACCCC | Novel | NAA |
| GAGGACAAACGGGCAACATAC | Novel | NNNRRT |
| GAGGACAGGAGGTTGGTGAG | Novel | NGA |
| GAGGCATAGCAGCAGGATGA | Novel | NGA |
| GAGGCATAGCAGCAGGATGA | Novel | NNAGGA |
| GAGTCCAAGGAATACTAACAT | Novel | NNNRRT |
| GATCCATACTGCGGAACTCC | Novel | NAG |
| GATGAGGCATAGCAGCAGGA | Novel | NGA |
| GCAAACACTTGGCACAGACC | Novel | NGG |
| GCAGACACATCCAGCGATAA | Novel | NNAGGA |
| GCAGACCAATTTATGCCTAC | Novel | NGC |
| GCAGAGTCTAGACTCGTGGT | M65 | NGA |
| GCATAGCAGCAGGATGAAGA | Novel | NGA |
| GCCGCAGACACATCCAGCGA | Novel | NAA |
| GCCGCAGACACATCCAGCGA | Novel | NNACCA |
| GCGAATCCACACTCCGAAAG | Novel | NNACCA |
| GCGAGCAAAACAAGCGGCTA | Novel | NGA |
| GCGCCGACGGGACGTAAACA | Novel | NAG |
| GCGTCAGCAAACACTTGGCA | Novel | NAG |
| GCTCAGGAGACTCTAAGGCTT | Novel | NNNRRT |
| GCTCCTCTGCCGATCCATAC | Novel | NGC |
| GCTGCGAGCAAAACAAGCGG | Novel | NNAGGA |
| GCTGCTATGCCTCATCTTCTT | E14 | NNNRRT |
| GGACTTCTCTCAATTTTCTA | E15 | NGG |
| GGAGTTCCGCAGTATGGATC | Novel | NGC |
| GGCACTAGTAAACTGAGCCA | Novel | NGA |
| GGCATAGCAGCAGGATGAAG | Novel | NGG |
| GGCGGGGTTTTTCTTGTTGAC | Novel | NNGRRT |
| GGCGGGGTTTTTCTTGTTGAC | Novel | NNNRRT |
| GGGACTGCGAATTTTGGCCA | Novel | NGA |
| GGGGACTGCGAATTTTGGCC | Novel | NAG |
| GGGGCATTTGGTGGTCTATA | Novel | NGC |
| GGGGCGCACCTCTCTTTACG | Novel | NGG |
| GGGTTGCGTCAGCAAACACT | Novel | NGG |
| GGTATACATTTAAACCCTAA | Novel | NAA |
| GGTTGCGTCAGCAAACACTT | Novel | NGC |
| GTAGCTCCAAATTCTTTATA | Novel | NGG |
| GTATACATTTAAACCCTAAC | Novel | NAA |
| GTCCAAGAGTCCTCTTATGT | Novel | NAG |
| GTCCAAGGAATACTAACATT | Novel | NAG |
| GTCCACCACGAGTCTAGACTC | E6/M2 | NNNRRT |
| GTCCATGCCCCAAAGCCACC | Novel | NAA |
| GTCCCGTCGGCGCTGAATCC | Novel | NGC |
| GTCCTTTGTTTACGTCCCGT | Novel | NGG |
| GTCGCAGAAGATCTCAATCT | Novel | NGG |
| GTCTGTGCCTTCTCATCTGC | Novel | NGG |
| GTGCCAAGTGTTTGCTGACG | Novel | NAA |
| GTGGACTTCTCTCAATTTTC | Novel | NAG |
| GTTAATCATTACTTCCAAAC | Novel | NAG |
| GTTACCAATTTTCTTTTGTCT | Novel | NNGRRT |
| GTTACCAATTTTCTTTTGTCT | Novel | NNNRRT |
| GTTATCGCTGGATGTGTCTG | Novel | NGG |
| GTTCCGCAGTATGGATCGGC | E17 | NGA |
| GTTCCGCAGTATGGATCGGC | Novel | NNAGGA |
| GTTTACTAGTGCCATTTGTT | Novel | NAG |
| GTTTACTAGTGCCATTTGTTC | Novel | NNNRRT |
| TAAAACGCCGCAGACACATC | Novel | NAG |
| TAAAACGCCGCAGACACATCC | E18 | NNNRRT |
| TACCGCAGAGTCTAGACTCG | M34 | NGG |
| TAGCAGCAGGATGAAGAGGAA | Novel | NNNRRT |
| TAGCCGCTTGTTTTGCTCGC | Novel | NGC |
| TAGCCGCTTGTTTTGCTCGCA | Novel | NNNRRT |
| TAGGCAGAGGTGAAAAAGTTG | Novel | NNNRRT |
| TAGGGCTTTCCCCCACTGTT | Novel | NGG |
| TAGTATTCCTTGGACTCATA | Novel | NGG |
| TATACATTTAAACCCTAACA | Novel | NAA |
| TATGTTGCCCGTTTGTCCTC | Novel | NAA |
| TATTCCTTGGACTCATAAGG | Novel | NGG |
| TCAGTTTACTAGTGCCATTTG | Novel | NNNRRT |
| TCCACCACGAGTCTAGACTC | Novel | NGC |
| TCCCCCTAGAAAATTGAGAG | Novel | NAG |
| TCCGCAGTATGGATCGGCAG | E19 | NGG |
| TCCTAGCCGCTTGTTTTGCT | Novel | NGC |
| TCCTCTGCCGATCCATACTG | E20 | NGG |
| TCCTCTTCATCCTGCTGCTA | Novel | NGC |
| TCGCAGAAGATCTCAATCTC | Novel | NGG |
| TCTCAATCGCCGCGTCGCAG | Novel | NAG |
| TCTTCTGCGACGCGGCGATT | Novel | NAG |
| TCTTGTTCCCAAGAATATGG | Novel | NGA |
| TGAAAGCCAAACAGTGGGGG | Novel | NAA |
| TGACGCAACCCCCACTGGCT | Novel | NGG |
| TGAGCCTGAGGGCTCCACCC | Novel | NAA |
| TGAGGCATAGCAGCAGGATG | Novel | NAG |
| TGCCCAAGGTCTTACATAAG | Novel | NGG |
| TGCCCGTTTGTCCTCTAATT | Novel | NNAGGA |
| TGCCCGTTTGTCCTCTAATTC | Novel | NNGRRT |
| TGCCCGTTTGTCCTCTAATTC | Novel | NNNRRT |
| TGCGAGCAAAACAAGCGGCT | Novel | NGG |
| TGCTGGTGGCTCCAGTTCAGG | Novel | NNNRRT |
| TGGACTTCTCTCAATTTTCT | E21 | NGG |
| TGGCCAAAATTCGCAGTCCC | Novel | NAA |
| TGGGGACTGCGAATTTTGGC | Novel | NAA |
| TGGTTATCGCTGGATGTGTC | Novel | NGC |
| TGTGCACTTCGCTTCACCTC | Novel | NGC |
| TGTGTCTTGGCCAAAATTCG | Novel | NAG |
| TTAAACCCTAACAAAACAAA | Novel | NAG |
| TTACTCTCGTTTTTGCCTTC | Novel | NGA |
| TTAGGCAGAGGTGAAAAAGT | Novel | NGC |
| TTATCGCTGGATGTGTCTGC | Novel | NGC |
| TTCCCCCACTGTTTGGCTTT | Novel | NAG |
| TTCCCGAGATTGAGATCTTC | Novel | NGC |
| TTCCGCAGTATGGATCGGCA | Novel | NAG |
| TTCGCTTCACCTCTGCACGT | Novel | NGC |
| TTGCTGACGCAACCCCCACT | Novel | NGC |
| TTGGAGGACAGGAGGTTGGTG | Novel | NNNRRT |
| TTTGCTGACGCAACCCCCAC | E23 | NGG |

Table 24 provides a list of sequences targeted by guide RNAs that introduce premature STOP codons into HBV genes.

TABLE 24
STOP gRNA List

| guide_seq | guide_id | pam |
|---|---|---|
| AACAAGATCTACAGCATGGGG | M21 | NNGRRT |
| AACCCCATCTCTTTGTTTTGT | M27 | NNGRRT |
| AAGCCACCCAAGGCACAGCT | M39 (precore) | NGG |
| AAGCCCAGGATGATGGGATG | M68 | NGA |
| ACACATCCAGCGATAACCAGG | M3 | NNNRRT |
| ACAGGCGGGGTTTTTCTTGT | M64 | NGA |
| ACCAGGACAAGTTGGAGGAC | M57 | NGG |
| ACCAGGACAAGTTGGAGGACA | M25 | NNNRRT |
| ACGAACCACTGAACAAATGGC | M5 | NNNRRT |
| ACGCCAACAAGGTAGGAGCT | M82 | NGA |
| AGCCACCAGCAGGGAAATAC | M56 | NGG |
| AGCCACCCAAGGCACAGCTT | M72 | NGA |
| AGGCAAGCAATTCTTTGCTG | M43 | NGG |
| AGGGTCCCCAATCCTCGAGA | M86 | NGA |
| AGTCCAAGGAATACTAACAT | M78 | NGA |
| ATTTACACCAAGACATTATCA | M16 | NNNRRT |
| CAACGAATTGTGGGTCTTTT | M62 | NGG |
| CAATACCGCAGAGTCTAGACT | M1 | NNNRRT |
| CACCACGAGTCTAGACTCTG | M36 | NGG |
| CAGGCAAGCAATTCTTTGCT | M42 | NGG |
| CATCCATATAACTGAAAGCCA | M6 | NNNRRT |
| CATGCAACTTTTTCACCTCTG | M8 | NNNRRT |
| CCAATCCTCGAGAAGATTGA | M84 | NGA |
| CCACCAATCGCCAGACAGGA | M55 | NGG |
| CCACCAGCACGGGACCATGC | M90 | NGA |
| CCACCCAAGGCACAGCTTGG | M38 | NGG |
| CCACTCCCATAGGAATTTTC | M69 | NGA |
| CCAGCAAAGAATTGCTTGCC | M73 | NGA |
| CCAGCCTTCAGAGCAAACACA | M22 | NNNRRT |
| CCAGGACAAGTTGGAGGACA | M88 | NGA |
| CCATGCCCCAAAGCCACCCA | M40 (precore) | NGG |
| CCCAACAAGGACACCTGGCC | M81 | NGA |
| CCCACCCAGGTAGCTAGAGTC | M11 | NNNRRT |
| CCCATCTCTTTGTTTTGTTA | M59 | NGG |
| CCCCAGCAAAGAATTGCTTGC | M10 | NNGRRT |
| CCCCATCTCTTTGTTTTGTT | M60 | NGG |
| CCGCCTTCCATAGAGTGTGTA | M19 | NNNRRT |
| CCGGCAACGGCCAGGTCTGTG | M32 | NNNRRT |
| CCTCCACCAATCGCCAGACA | M83 | NGA |
| CGATAACCAGGACAAGTTGG | M58 | NGG |
| CGCAGAGTCTAGACTCGTGG | M35 | NGG |
| CTCAATCGCCGCGTCGCAGA | M77 | NGA |
| CTCCATGCGACGTGCAGAGG | M92 | NGA |
| CTGAGCCAGGAGAAACGGGC | M70 | NGA |
| CTGCCAACTGGATCCTGCGC | M191 | NGG |
| CTTCTCTCAATTTTCTAGGG | M87 | NGA |
| GAAAGCCCAGGATGATGGGA | M37 | NGG |
| GAAAGCCCAGGATGATGGGAT | M4 | NNGRRT |
| GAAGATCTCAATCTCGGGAAC | M14 | NNNRRT |
| GAATCCACACTCCGAAAGACA | M12 | NNNRRT |
| GACACATCCAGCGATAACCA | M67 | NGA |
| GACGACGAGGCAGGTCCCCT | M74 | NGA |
| GACGAGGCAGGTCCCCTAGA | M75 | NGA |
| GACGCCAACAAGGTAGGAGC | M53 | NGG |
| GAGAAGTCCACCACGAGTCT | M66 | NGA |
| GATAACCAGGACAAGTTGGA | M89 | NGA |
| GATTGCAATTGATTATGCCTG | M18 | NNNRRT |
| GCAAGCAATTCTTTGCTGGG | M45 | NGG |
| GCAGAGTCTAGACTCGTGGT | M65 | NGA |
| GCCACAAGAACACATCATACA | M28 | NNNRRT |
| GCTGCCAACTGGATCCTGCG | M190 | NGG |
| GCTGTACCAAACCTTCGGAC | M91 | NGA |
| GGAACAAGATCTACAGCATG | M51 | NGG |
| GGCAAGCAATTCTTTGCTGG | M44 | NGG |
| GGGAACAAGATCTACAGCAT | M50 | NGG |
| GGTCTCAATCGCCGCGTCGC | M76 | NGA |
| GGTCTCAATCGCCGCGTCGCA | M13 | NNNRRT |
| GGTCTCCATGCGACGTGCAG | M63 | NGG |
| GGTGGTCTCCATGCGACGTGC | M33 | NNNRRT |
| GTCCACCACGAGTCTAGACTC | E16/M2 | NNNRRT |
| GTGGTCTCCATGCGACGTGC | M93 | NGA |
| GTTTTCCAATGAGGATTAAAG | M15 | NNNRRT |
| TACACAATGTGGTTATCCTGC | M31 | NNNRRT |
| TACCACATCATCCATATAAC | M71 | NGA |
| TACCGCAGAGTCTAGACTCG | M34 | NGG |
| TACTTTCCAATCAATAGGCCT | M29 | NNNRRT |
| TATACCCAAAGACAAAAGAAA | M7 | NNNRRT |
| TCAACGAATTGTGGGTCTTT | M61 | NGG |
| TCAATCCCAACAAGGACACC | M52 | NGG |
| TCAGGCAAGCAATTCTTTGC | M41 | NGG |
| TCCAAGGAATACTAACATTG | M46 | NGG |
| TCCCAAGAATATGGTGACCCA | M20 | NNNRRT |
| TCCCCAATCCTCGAGAAGAT | M85 | NGA |
| TCCCCAATCCTCGAGAAGATT | M24 | NNNRRT |
| TGAACAGTTTGTAGGCCCACT | M17 | NNNRRT |
| TGCAATTGATTATGCCTGCT | M48 | NGG |
| TGCCAACTGGATCCTGCGCG | M189 | NGA |
| TGCTCCAGCTCCTACCTTGT | M54 | NGG |
| TGCTGTACCAAACCTTCGGAC | M26 | NNNRRT |
| TGGGAACAAGATCTACAGCA | M49 | NGG |
| TGTCAACGAATTGTGGGTCTT | M30 | NNGRRT |
| TGTTTTCCAATGAGGATTAA | M79 | NGA |
| TTCAAGCCTCCAAGCTGTGCC | E22/M9 | NNGRRT |
| TTCAGAGCAAACACAGCAAAT | M23 | NNNRRT |
| TTCCAATGAGGATTAAAGAC | M47 | NGG |
| TTTCCACCAGCAATCCTCTG | M80 | NGA |
| AAAGCCAAACAGTGGGGGAA | Novel | NGC |
| AAAGCCCAGGATGATGGGAT | Novel | NGG |
| AACAAGATCTACAGCATGGGG | Novel | NNNRRT |
| AACCACTGAACAAATGGCAC | Novel | NAG |
| AACCAGGACAAGTTGGAGGA | Novel | NAG |
| AACCCCATCTCTTTGTTTTGT | Novel | NNNRRT |
| AACTCTGCAAGATCCCAGAG | Novel | NGA |
| AAGCCCCAGCCAGTGGGGGT | Novel | NGC |
| AATGTATACCCAAAGACAAAA | Novel | NNNRRT |
| AATTCGCAGTCCCCAACCTC | Novel | NAA |
| ACATCATCCATATAACTGAA | Novel | NGC |
| ACATCCAGCGATAACCAGGA | Novel | NAA |
| ACCCAAAGACAAAAGAAAAT | Novel | NGG |
| ACCCAGGTAGCTAGAGTCAT | Novel | NAG |
| ACCCCATCTCTTTGTTTTGT | Novel | NAG |
| ACCTCAATGTTAGTATTCCT | Novel | NGG |
| ACGACGAGGCAGGTCCCCTA | Novel | NAA |
| ACTCCCATAGGAATTTTCCG | Novel | NAA |
| ACTCCTCCCAGTCTTTAAAC | Novel | NAA |
| ACTCCTCCCAGTCTTTAAACA | E96 | NNNRRT |
| ACTCTGCAAGATCCCAGAGT | Novel | NAG |
| AGACGACGAGGCAGGTCCCC | Novel | NAG |
| AGATTGCAATTGATTATGCC | Novel | NGC |
| AGCCAGGAGAAACGGGCTGA | Novel | NGC |
| AGCCCAGGATGATGGGATGG | Novel | NAA |
| AGCCTTCAGAGCAAACACAG | Novel | NAA |
| AGCGATAACCAGGACAAGTT | Novel | NNAGGA |
| AGGTCTCAATCGCCGCGTCG | Novel | NAG |
| ATACTACAAACTTTGCCAGC | Novel | NAA |
| ATCCACACTCCGAAAGACAC | Novel | NAA |
| ATCCAGTTGGCAGCACAGCC | Novel | NAG |
| ATGGGGCAGAATCTTTCCAC | Novel | NAG |
| ATGGGGCAGAATCTTTCCACC | Novel | NNNRRT |
| ATTTGTTCAGTGGTTCGTAG | Novel | NGC |
| ATTTTGGCCAAGACACACGG | Novel | NAG |
| CAAAATTCGCAGTCCCCAACC | Novel | NNNRRT |
| CAACCACCAGCACGGGACCA | Novel | NGC |
| CAATCCCAACAAGGACACCT | Novel | NGC |
| CAATCGCCAGACAGGAAGGC | Novel | NGC |
| CACCAAACTCTGCAAGATCC | Novel | NAG |
| CACCAATCGCCAGACAGGAA | Novel | NGC |
| CACCAGCACGGGACCATGCC | Novel | NAA |
| CACCAGTTGGATCCAGCCTT | Novel | NAG |
| CACTCCCATAGGAATTTTCC | Novel | NAA |
| CAGACGAAGGTCTCAATCGC | Novel | NGC |
| CAGGATCCAGTTGGCAGCAC | Novel | NGC |
| CATACTACAAACTTTGCCAG | Novel | NAA |
| CATCCAGCGATAACCAGGAC | Novel | NAG |
| CATCCATATAACTGAAAGCC | Novel | NAA |
| CCAATACCACATCATCCATA | Novel | NAA |
| CCACAAGAACACATCATACA | Novel | NAA |
| CCCCAGCAAAGAATTGCTTGC | Novel | NNNRRT |
| CCCCCCAGCAAAGAATTGCT | Novel | NGC |
| CCCGGCAACGGCCAGGTCTG | Novel | NGC |
| CCGCCTTCCATAGAGTGTGT | Novel | NAA |
| CCTCAATGTTAGTATTCCTT | Novel | NGA |
| CCTCCCAGTCTTTAAACAAA | Novel | NAG |
| CCTTCCATAGAGTGTGTAAA | Novel | NAG |
| CGACGAGGCAGGTCCCCTAG | Novel | NAG |
| CGCCAACAAGGTAGGAGCTG | Novel | NAG |
| CGCCCACCGAATGTTGCCCA | E95 | NGG |
| CGGACGACCCTTCTCGGGGT | Novel | NGC |
| CGTCTGGCCAGGTGTCCTTGT | Novel | NNGRRT |
| CGTCTGGCCAGGTGTCCTTGT | Novel | NNNRRT |
| CGTTCCGACCGACCACGGGG | Novel | NGC |
| CTACGAACCACTGAACAAAT | Novel | NGC |
| CTCAATCTCGGGAACCTCAAT | Novel | NNNRRT |
| CTCCACCAATCGCCAGACAG | Novel | NAA |
| CTCCACCCCAAAAGGCCTCCG | Novel | NNNRRT |
| CTCCCATAGGAATTTTCCGA | Novel | NAG |
| CTCTGCAAGATCCCAGAGTG | Novel | NGA |
| CTGAAAGCCAAACAGTGGGG | Novel | NAA |
| CTGCAAGATCCCAGAGTGAG | Novel | NGG |
| CTGGCCAGGTGTCCTTGTTG | Novel | NGA |
| CTGTACCAAACCTTCGGACG | Novel | NAA |
| CTGTGCCAAGTGTTTGCTGA | Novel | NGC |
| GAAAGCCAAACAGTGGGGGA | Novel | NAG |
| GAAAGCCCAGGATGATGGGAT | Novel | NNNRRT |
| GAACAAGATCTACAGCATGG | Novel | NGC |
| GAGCCACCAGCAGGGAAATA | Novel | NAG |
| GAGCCAGGAGAAACGGGCTG | Novel | NGG |
| GAGTCCAAGGAATACTAACAT | Novel | NNNRRT |
| GATCTCAATCTCGGGAACCT | Novel | NAA |
| GCAGGATCCAGTTGGCAGCA | Novel | NAG |
| GCCAACAAGGTAGGAGCTGG | Novel | NGC |
| GCCACAAGAACACATCATAC | Novel | NAA |
| GCCACCAGCAGGGAAATACA | Novel | NGC |
| GCCAGGTGTCCTTGTTGGGAT | Novel | NNNRRT |
| GCCGTTCCGACCGACCACGG | Novel | NGC |
| GCCTTCAGAGCAAACACAGC | Novel | NAA |
| GCGAATCCACACTCCGAAAG | Novel | NNACCA |
| GCGATAACCAGGACAAGTTG | Novel | NAG |
| GCTCCAGCTCCTACCTTGTT | Novel | NGC |
| GGCATACTACAAACTTTGCCA | Novel | NNNRRT |
| GGCTCCAGTTCAGGAGCAGT | Novel | NAA |
| GGGCAGAATCTTTCCACCAG | Novel | NAA |
| GGGCCATCAGCGCGTGCGTG | Novel | NAA |
| GTACGAGATCTTCTAGATAC | Novel | NGC |
| GTATACCCAAAGACAAAAGA | Novel | NAA |
| GTCCAAGGAATACTAACATT | Novel | NAG |
| GTCTCAATCGCCGCGTCGCA | Novel | NAA |
| GTCTGGCCAGGTGTCCTTGT | Novel | NGG |
| GTGAAACCACAAGAGTTGCC | Novel | NGA |
| GTGCCAAGTGTTTGCTGACG | Novel | NAA |
| GTGCTGCCAACTGGATCCTG | Novel | NGC |
| GTGTTTTCCAATGAGGATTA | Novel | NAG |
| TAACCAGGACAAGTTGGAGG | Novel | NNAGGA |
| TAAGCAGGCTTTCACTTTCT | Novel | NGC |
| TACACCAAGACATTATCAAA | Novel | NAA |
| TCACCAAACTCTGCAAGATCC | Novel | NNGRRT |
| TCACCAAACTCTGCAAGATCC | Novel | NNNRRT |
| TCAGGCTCAGGGCATACTAC | Novel | NAA |
| TCATCCATATAACTGAAAGC | Novel | NAA |
| TCCAATCAATAGGCCTGTTA | Novel | NNAGGA |
| TCCACCAATCGCCAGACAGG | Novel | NAG |
| TCCACCACGAGTCTAGACTC | Novel | NGC |
| TCCCAACAAGGACACCTGGC | Novel | NAG |
| TCCCATAGGAATTTTCCGAA | Novel | NGC |
| TCCCGACCACCAGTTGGATC | Novel | NAG |
| TCGCAGACGAAGGTCTCAAT | Novel | NGC |
| TCTCAATCGCCGCGTCGCAG | Novel | NAG |
| TCTGCAAGATCCCAGAGTGA | Novel | NAG |
| TCTGGCCAGGTGTCCTTGTT | Novel | NGG |
| TGAAACCACAAGAGTTGCCT | Novel | NAA |
| TGAAAGCCAAACAGTGGGGG | Novel | NAA |
| TGAGCCAGGAGAAACGGGCT | Novel | NAG |
| TGCCACAAGAACACATCATA | Novel | NAA |
| TGCCGAACCTGCATGACTAC | Novel | NGC |
| TGGCCAAAATTCGCAGTCCC | Novel | NAA |
| TGGCTCCAGTTCAGGAGCAG | Novel | NAA |
| TGGGGCAGAATCTTTCCACC | Novel | NGC |
| TGGTCTCCATGCGACGTGCA | Novel | NAG |
| TGTACCAAACCTTCGGACGG | Novel | NAA |
| TGTATACCCAAAGACAAAAG | Novel | NAA |
| TGTCAACGAATTGTGGGTCTT | Novel | NNNRRT |
| TTACACCAAGACATTATCAA | Novel | NAA |
| TTCTCTCAATTTTCTAGGGG | Novel | NAA |
| TTGCAATTGATTATGCCTGC | Novel | NAG |
| TTTACACAATGTGGTTATCC | Novel | NGC |
| TTTACACCAAGACATTATCA | Novel | NAA |
| TTTCCAATCAATAGGCCTGT | Novel | NAA |
| TTTCCAATGAGGATTAAAGA | Novel | NAG |

Table 25 is a list of sequences targeted by guide RNAs in the HBV genome.

TABLE 25
gRNAs for targeting the HBV genome

| guide_seq | guide_id | pam |
|---|---|---|
| AAAAAATCAAAGAATGTTTT | Novel | NGA |
| AAAAAATGTGAACAGTTTGT | Novel | NGG |
| AAAAACCCCGCCTGTAACAC | Novel | NAG |
| AAAAAGTTGCATGGTGCTGG | Novel | NGC |
| AAAAATCAAAGAATGTTTTA | Novel | NAA |
| AAAAATGTGAACAGTTTGTA | Novel | NGC |
| AAAACAAGCGGCTAGGAGTTC | Novel | NNNRRT |
| AAAACATTCTTTGATTTTTTG | Novel | NNNRRT |
| AAAACCCCGCCTGTAACACG | Novel | NGA |
| AAAACGCCGCAGACACATCC | Novel | NGC |
| AAAAGATGGTGTTTTCCAAT | Novel | NAG |
| AAAAGGTTCCACGCACGCGC | Novel | NGA |
| AAAAGTGAGACAAGAAATGT | Novel | NAA |
| AAAATCAAAGAATGTTTTAG | Novel | NAA |
| AAAATTGGTAACAGCGGTAA | Novel | NAA |
| AAACAAAGGACGTCCCGCGC | Novel | NGG |
| AAACAAGCGGCTAGGAGTTC | Novel | NGC |
| AAACACAGCAAATCCAGATT | Novel | NGG |
| AAACACTCATCCTCAGGCCA | Novel | NGC |
| AAACCCAGCCCGAATGCTCC | Novel | NGC |
| AAACCCCGCCTGTAACACGA | Novel | NAA |
| AAACGAGAGTAACTCCACAG | Novel | NAG |
| AAACGGGCAACATACCTTGA | Novel | NAG |
| AAACTACTGTTGTTAGACGA | Novel | NGA |
| AAACTGAGCCAGGAGAAACG | Novel | NGC |
| AAACTGTTCACATTTTTTGA | Novel | NAA |
| AAACTTCCTATTAACAGGCCT | Novel | NNNRRT |
| AAAGAATCCCAGAGGATTGC | Novel | NGG |
| AAAGAATTTGGAGCTACTGT | Novel | NGA |
| AAAGACTGGGAGGAGTTGGG | Novel | NGA |
| AAAGACTGGGAGGAGTTGGG | Novel | NNAGGA |
| AAAGAGATGGGGTTACTCTC | Novel | NGA |
| AAAGATGGTGTTTTCCAATG | Novel | NGG |
| AAAGATTCTGCCCCATGCTG | Novel | NAG |
| AAAGCCAAACAGTGGGGGAA | Novel | NGC |
| AAAGCCCAGGATGATGGGAT | Novel | NGG |
| AAAGGCCTTGTAAGTTGGCG | Novel | NGA |
| AAAGGCCTTGTAAGTTGGCGA | Novel | NNNRRT |
| AAAGGTGGAGACAGCGGGGT | Novel | NGG |
| AAAGTATGTCAACGAATTGT | Novel | NGG |
| AAAGTGAGACAAGAAATGTG | Novel | NAA |
| AAAGTGAGACAAGAAATGTG | Novel | NNACCA |
| AAAGTTGCATGGTGCTGGTG | Novel | NGC |
| AAAGTTTGTAGTATGCCCTG | Novel | NGC |
| AAATACAGGCCTCTCACTCT | Novel | NGG |
| AAATATTTACCATTGGATAA | Novel | NGG |
| AAATCAAAGAATGTTTTAGA | Novel | NAA |
| AAATGGGGCAGCAAAACCCA | Novel | NAA |
| AAATGTATACCCAAAGACAA | Novel | NAG |
| AAATGTATATTAGGAAAAGA | Novel | NGG |
| AAATGTGAAACCACAAGAGT | Novel | NGC |
| AAATTAACACCCACCCAGGT | Novel | NGC |
| AAATTGAGAGAAGTCCACCA | Novel | NGA |
| AAATTGGTAACAGCGGTAAA | Novel | NAG |
| AACAAACAGTCTTTGAAGTA | Novel | NGC |
| AACAAAGGACGTCCCGCGCA | Novel | NGA |
| AACAAATGGCACTAGTAAAC | Novel | NGA |
| AACAAGAAAAACCCCGCCTG | Novel | NAA |
| AACAAGAAGATGAGGCATAG | Novel | NAG |
| AACAAGAGATGATTAGGCAG | Novel | NGG |
| AACAAGATCTACAGCATGGGG | M21 | NNGRRT |
| AACAAGATCTACAGCATGGGG | Novel | NNNRRT |
| AACAAGGACACCTGGCCAGA | Novel | NGC |
| AACAATGCTCAGGAGACTCT | Novel | NAG |
| AACACAGCAAATCCAGATTG | Novel | NGA |
| AACACATCATACAAAAAATC | Novel | NAA |
| AACACATCATACAAAAAATCA | Novel | NNGRRT |
| AACACATCATACAAAAAATCA | Novel | NNNRRT |
| AACACCCACCCAGGTAGCTA | Novel | NAG |
| AACACGAGAAGGGGTCCTAG | Novel | NAA |
| AACAGAGTTATCAGTCCCGA | Novel | NAA |
| AACAGCGGTAAAAAGGGACT | Novel | NAA |
| AACAGTAGGACATGAACAAG | Novel | NGA |
| AACAGTAGGACATGAACAAGA | Novel | NNNRRT |
| AACAGTCTTTGAAGTATGCCT | Novel | NNNRRT |
| AACAGTGGGGGAAAGCCCTA | Novel | NGA |
| AACATACCTTGATAGTCCAG | Novel | NAG |
| AACATCACATCAGGATTCCT | Novel | NGG |
| AACATGGAGAACATCACATC | Novel | NGG |
| AACATTGAGGTTCCCGAGAT | Novel | NGA |
| AACCACTGAACAAATGGCAC | Novel | NAG |
| AACCAGGACAAGTTGGAGGA | Novel | NAG |
| AACCCATAAAATTCAGAGAG | Novel | NAA |
| AACCCCATCTCTTTGTTTTGT | M27 | NNGRRT |
| AACCCCATCTCTTTGTTTTGT | Novel | NNNRRT |
| AACCCCCACTGGCTGGGGCT | Novel | NGG |
| AACCCCGCCTGTAACACGAG | Novel | NAG |
| AACCCCGCCTGTAACACGAGA | Novel | NNGRRT |
| AACCCCGCCTGTAACACGAGA | Novel | NNNRRT |
| AACCCTAACAAAACAAAGAGA | Novel | NNGRRT |
| AACCCTAACAAAACAAAGAGA | Novel | NNNRRT |
| AACCTAGCAGGCATAATCAAT | Novel | NNNRRT |
| AACCTGCATGACTACTGCTC | Novel | NAG |
| AACCTTTCACCAAACTCTGC | Novel | NAG |
| AACCTTTGGATAAAACCTAG | Novel | NAG |
| AACCTTTTCGGCTCCTCTGC | Novel | NGA |
| AACGAGAGTAACTCCACAGT | Novel | NGC |
| AACGCAGGATAACCACATTGT | Novel | NNNRRT |
| AACGCCCACCGAATGTTGCC | Novel | NAA |
| AACGCCGCAGACACATCCAG | Novel | NGA |
| AACGGTTTCTCTTCCAAAAG | Novel | NGA |
| AACTAATGACTCTAGCTACC | Novel | NGG |
| AACTACCGTGTGTCTTGGCC | Novel | NAA |
| AACTACTGTTGTTAGACGAC | Novel | NAG |
| AACTAGATGTTCTGGATAAT | Novel | NAG |
| AACTCCCTCGCCTCGCAGAC | Novel | NAA |
| AACTCCTCCCAGTCTTTAAA | Novel | NAA |
| AACTCTGCAAGATCCCAGAG | Novel | NGA |
| AACTCTGTTGTCCTCTCCCG | Novel | NAA |
| AACTGAAAGCCAAACAGTGG | Novel | NGG |
| AACTGGAGCCACCAGCAGGG | Novel | NAA |
| AACTTCCAATGACATAACCCA | Novel | NNNRRT |
| AACTTGTCCTGGTTATCGCT | Novel | NGA |
| AAGAAAATTGGTAACAGCGG | Novel | NAA |
| AAGAACCAACAAGAAGATGA | Novel | NGC |
| AAGAAGATGAGGCATAGCAG | Novel | NAG |
| AAGAAGATTGCAATTGATTA | Novel | NGC |
| AAGAAGTCAGAAGGCAAAAA | Novel | NGA |
| AAGAAGTCAGAAGGCAAAAAC | Novel | NNGRRT |
| AAGAAGTCAGAAGGCAAAAAC | Novel | NNNRRT |
| AAGAATCCTCACAATACCGC | Novel | NGA |
| AAGAATTGCTTGCCTGAGTG | Novel | NAG |
| AAGAATTTGGAGCTACTGTG | Novel | NAG |
| AAGACATTATCAAAAAATGT | Novel | NAA |
| AAGACATTATCAAAAAATGTG | Novel | NNNRRT |
| AAGACTGGGAGGAGTTGGGG | Novel | NAG |
| AAGACTGTTTGTTTAAAGAC | Novel | NGG |
| AAGAGAAACCGTTATAGAGTA | Novel | NNNRRT |
| AAGAGAGAAACAACACATAG | Novel | NGC |
| AAGAGATGATTAGGCAGAGG | Novel | NGA |
| AAGAGATGGGGTTACTCTCT | Novel | NAA |
| AAGAGGACTCTTGGACTCTC | Novel | NGC |
| AAGATCTACAGCATGGGGCA | Novel | NAA |
| AAGATCTCGTACTGAAGGAA | Novel | NGA |
| AAGATGGTGTTTTCCAATGA | Novel | NGA |
| AAGATTCTGCCCCATGCTGT | Novel | NGA |
| AAGATTGACGATAAGGGAGA | Novel | NGC |
| AAGCAATTCTTTGCTGGGGG | Novel | NAA |
| AAGCCACCCAAGGCACAGCT | M39 | NGG |
| AAGCCCAGGATGATGGGATG | M68 | NGA |
| AAGCCCCAGCCAGTGGGGGT | Novel | NGC |
| AAGCCCTACGAACCACTGAA | Novel | NAA |
| AAGCCTCCAAGCTGTGCCTT | Novel | NGG |
| AAGCGAAGTGCACACGGTCC | Novel | NGC |
| AAGCGAAGTGCACACGGTCCG | Novel | NNNRRT |
| AAGGAAAGAAGTCAGAAGGC | Novel | NAA |
| AAGGACACCTGGCCAGACGC | Novel | NAA |
| AAGGCACAGCTTGGAGGCTT | Novel | NAA |
| AAGGCACAGCTTGGAGGCTTG | E1 | NNNRRT |
| AAGGCCTCCGTGCGGTGGGG | Novel | NGA |
| AAGGCCTTGTAAGTTGGCGA | Novel | NAA |
| AAGGCGGGTATATTATATAA | Novel | NAG |
| AAGGCTTCCCGATACAGAGC | Novel | NGA |
| AAGGGACTCAAGATGCTGTA | Novel | NAG |
| AAGGGGACGAGAGAGTCCCA | Novel | NGC |
| AAGGGGTCCTAGGAATCCTGA | Novel | NNNRRT |
| AAGGGTCGATGTCCATGCCC | Novel | NAA |
| AAGGGTCGTCCGCAGGATTC | Novel | NGC |
| AAGGTAGGAGCTGGAGCATT | Novel | NGG |
| AAGGTCGGTCGTTGACATTG | Novel | NAG |
| AAGGTGGAGACAGCGGGGTA | Novel | NGC |
| AAGGTTACCAAATATTTACCA | Novel | NNGRRT |
| AAGGTTACCAAATATTTACCA | Novel | NNNRRT |
| AAGGTTTGGTACAGCAACAG | Novel | NAG |
| AAGGTTTGGTACAGCAACAGG | Novel | NNGRRT |
| AAGGTTTGGTACAGCAACAGG | Novel | NNNRRT |
| AAGTCAGAAGGCAAAAACGA | Novel | NAG |
| AAGTGAAAGCCTGCTTAGAT | Novel | NGA |
| AAGTTATGGGTCCTTGCCAC | Novel | NAG |
| AAGTTGGAGGACAGGAGGTTG | Novel | NNGRRT |
| AAGTTGGAGGACAGGAGGTTG | Novel | NNNRRT |
| AAGTTTTCTAAAACATTCTT | Novel | NGA |
| AATACAGGCCTCTCACTCTG | Novel | NGA |
| AATACTAACATTGAGGTTCC | Novel | NGA |
| AATAGTGTCTAGTTTGGAAG | Novel | NAA |
| AATAGTGTCTAGTTTGGAAGT | Novel | NNNRRT |
| AATATGGTGACCCACAAAAT | Novel | NAG |
| AATATTTGGTAACCTTTGGA | Novel | NAA |
| AATCAATAGGCCTGTTAATA | Novel | NGA |
| AATCCCAGAGGATTGCTGGT | Novel | NGA |
| AATCCGCCTCCTGCCTCCAC | Novel | NAA |
| AATCCTCTGGGATTCTTTCC | Novel | NGA |
| AATCCTCTGGGATTCTTTCC | Novel | NNACCA |
| AATCCTGCGGACGACCCTTCT | Novel | NNGRRT |
| AATCCTGCGGACGACCCTTCT | Novel | NNNRRT |
| AATCGCCGCGTCGCAGAAGAT | Novel | NNNRRT |
| AATCTCGGGAACCTCAATGT | Novel | NAG |
| AATCTTCTTTTCTCATTAACT | Novel | NNNRRT |
| AATGACTCTAGCTACCTGGG | Novel | NGG |
| AATGATTAACTAGATGTTCT | Novel | NGA |
| AATGATTAACTAGATGTTCTG | Novel | NNNRRT |
| AATGGCACTAGTAAACTGAG | Novel | NNAGGA |
| AATGGGGCAGCAAAACCCAA | Novel | NAG |
| AATGTATACCCAAAGACAAA | Novel | NGA |
| AATGTATACCCAAAGACAAAA | Novel | NNNRRT |
| AATGTCAACGACCGACCTTG | Novel | NGG |
| AATGTTTGCTCCAGACCTGC | Novel | NGC |
| AATTCGCAGTCCCCAACCTC | Novel | NAA |
| AATTCGTTGACATACTTTCCA | Novel | NNNRRT |
| AATTCTTTGCTGGGGGGAAC | Novel | NAA |
| AATTGAGAGAAGTCCACCAC | Novel | NAG |
| AATTGGTAACAGCGGTAAAA | Novel | NGG |
| AATTTATGCCTACAGCCTCC | Novel | NAG |
| AATTTGGAAGATCCAGCATC | Novel | NAG |
| AATTTTATGGGTTATGTCAT | Novel | NGG |
| AATTTTATGGGTTATGTCATT | Novel | NNNRRT |
| AATTTTCCGAAAGCCCAGGA | Novel | NGA |
| ACAAACTTTGCCAGCAAATC | Novel | NGC |
| ACAAAGAGATGGGGTTACTCT | Novel | NNGRRT |
| ACAAAGAGATGGGGTTACTCT | Novel | NNNRRT |
| ACAAATGGCACTAGTAAACT | Novel | NAG |
| ACAACAGTAGTTTCCGGAAGT | Novel | NNNRRT |
| ACAACCTTTCACCAAACTCTG | Novel | NNNRRT |
| ACAAGAAATGTGAAACCACA | Novel | NGA |
| ACAAGAACACATCATACAAA | Novel | NAA |
| ACAAGAAGATGAGGCATAGC | Novel | NGC |
| ACAAGAATCCTCACAATACCG | Novel | NNGRRT |
| ACAAGAATCCTCACAATACCG | Novel | NNNRRT |
| ACAAGAGTTGCCTGAACTTT | Novel | NGG |
| ACAAGATCTACAGCATGGGG | Novel | NAG |
| ACAAGCGGCTAGGAGTTCCG | Novel | NAG |
| ACAAGGCCTTTCTGTGTAAA | Novel | NAA |
| ACAAGGGCATTAACGCAGGA | Novel | NAA |
| ACAAGGGCATTAACGCAGGA | Novel | NNACCA |
| ACAATGCTCAGGAGACTCTA | Novel | NGG |
| ACAATGTGGTTATCCTGCGT | Novel | NAA |
| ACACACGGTAGTTCCCCCTA | Novel | NAA |
| ACACATAGCGCCTCATTTTG | Novel | NGG |
| ACACATCATACAAAAAATCA | Novel | NAG |
| ACACATCCAGCGATAACCAGG | M3 | NNNRRT |
| ACACCTGGCCAGACGCCAAC | Novel | NAG |
| ACACGGTAGTTCCCCCTAGA | Novel | NAA |
| ACACGGTCCGGCAGATGAGA | Novel | NGG |
| ACACTATTTACACACTCTAT | Novel | NGA |
| ACACTCATCCTCAGGCCATG | Novel | NAG |
| ACAGAAAGGCCTTGTAAGTT | Novel | NGC |
| ACAGACGGGGAGTCCGCGTA | Novel | NAG |
| ACAGAGCTGAGGCGGTATCT | Novel | NGA |
| ACAGAGCTGAGGCGGTATCTA | Novel | NNNRRT |
| ACAGCAAATCCAGATTGGGAC | Novel | NNNRRT |
| ACAGCAACAGGAGGGATACA | Novel | NAG |
| ACAGCAACAGGAGGGATACAT | Novel | NNNRRT |
| ACAGCGGTAAAAAGGGACTC | Novel | NAG |
| ACAGCTTGGAGGCTTGAACA | Novel | NNAGGA |
| ACAGGAGGTTGGTGAGTGAT | Novel | NGG |
| ACAGGAGGTTGGTGAGTGATT | Novel | NNNRRT |
| ACAGGCGGGGTTTTTCTTGT | M64 | NGA |
| ACAGGTACAGTAGAAGAATA | Novel | NAG |
| ACAGGTGCAATTTCCGTCCG | Novel | NAG |
| ACAGTAGTTTCCGGAAGTGT | Novel | NGA |
| ACAGTCTTTGAAGTATGCCT | Novel | NAA |
| ACAGTGGGGGAAAGCCCTAC | Novel | NAA |
| ACAGTGGGGGAAAGCCCTAC | Novel | NNACCA |
| ACAGTTTGTAGGCCCACTTA | Novel | NAG |
| ACATAACCCATAAAATTCAG | Novel | NGA |
| ACATAACTGACTACTAGGTCT | Novel | NNNRRT |
| ACATACCTTGATAGTCCAGA | Novel | NGA |
| ACATCACATCAGGATTCCTA | Novel | NGA |
| ACATCATACAAAAAATCAAA | Novel | NAA |
| ACATCATCCATATAACTGAA | Novel | NGC |
| ACATCCAGCGATAACCAGGA | Novel | NAA |
| ACATCGTATCCATGGCTGCT | Novel | NGG |
| ACATGAACAAGAGATGATTA | Novel | NGC |
| ACATGGAGAACATCACATCA | Novel | NGA |
| ACATTATCAAAAAATGTGAA | Novel | NAG |
| ACATTCTTTGATTTTTTGTA | Novel | NGA |
| ACATTGAGGTTCCCGAGATT | Novel | NAG |
| ACATTGTGTAAATGGGGCAG | Novel | NAA |
| ACATTTAAACCCTAACAAAA | Novel | NAA |
| ACATTTCTTGTCTCACTTTT | Novel | NGA |
| ACCAAACTCTGCAAGATCCC | Novel | NGA |
| ACCAAATATTTACCATTGGA | Novel | NAA |
| ACCAAATATTTACCATTGGAT | Novel | NNGRRT |
| ACCAAATATTTACCATTGGAT | Novel | NNNRRT |
| ACCAAATGCCCCTATCCTAT | Novel | NAA |
| ACCAACAAGAAGATGAGGCA | Novel | NAG |
| ACCAATTTATGCCTACAGCCT | E2 | NNNRRT |
| ACCAATTTTCTTTTGTCTTT | Novel | NGG |
| ACCACATCATCCATATAACT | Novel | NAA |
| ACCACATTGTGTAAATGGGG | Novel | NAG |
| ACCAGGACAAGTTGGAGGAC | M57 | NGG |
| ACCAGGACAAGTTGGAGGACA | M25 | NNNRRT |
| ACCAGTAAAGTTCCCCACCTT | Novel | NNGRRT |
| ACCAGTAAAGTTCCCCACCTT | Novel | NNNRRT |
| ACCAGTTGGATCCAGCCTTC | Novel | NGA |
| ACCCAAAGACAAAAGAAAAT | Novel | NGG |
| ACCCAGGTAGCTAGAGTCAT | Novel | NAG |
| ACCCATAACTTCCAATGACA | Novel | NAA |
| ACCCCATCTCTTTGTTTTGT | Novel | NAG |
| ACCCCGAGAAGGGTCGTCCG | Novel | NAG |
| ACCCCGCCTGTAACACGAGA | Novel | NGG |
| ACCCCGCTGTCTCCACCTTT | Novel | NAG |
| ACCCCTTCTCGTGTTACAGG | Novel | NGG |
| ACCCTAACAAAACAAAGAGA | Novel | NGG |
| ACCCTTATCCAATGGTAAATA | Novel | NNNRRT |
| ACCCTTCTCGGGGTCGCTTG | Novel | NGA |
| ACCGACCTTGAGGCATACTT | Novel | NAA |
| ACCGCCTCAGCTCTGTATCG | Novel | NGA |
| ACCTAGCAGGCATAATCAAT | Novel | NGC |
| ACCTCAATGTTAGTATTCCT | Novel | NGG |
| ACCTCACCATACTGCACTCA | Novel | NGC |
| ACCTCCTGTCCTCCAACTTGT | Novel | NNNRRT |
| ACCTGCATGACTACTGCTCA | Novel | NGG |
| ACCTGGCCGTTGCCGGGCAA | Novel | NGG |
| ACCTGGGTGGGTGTTAATTT | Novel | NGA |
| ACCTGGGTGGGTGTTAATTTG | Novel | NNNRRT |
| ACCTGTCTTTAATCCTCATT | Novel | NGA |
| ACCTTATTATCCAGAACATC | Novel | NAG |
| ACCTTCGTCTGCGAGGCGAG | Novel | NGA |
| ACCTTGGGCAACATTCGGTG | Novel | NGC |
| ACCTTTACCCCGTTGCCCGG | Novel | NAA |
| ACCTTTCACCAAACTCTGCA | Novel | NGA |
| ACCTTTGGATAAAACCTAGC | Novel | NGG |
| ACGAACCACTGAACAAATGGC | M5 | NNNRRT |
| ACGACGAGGCAGGTCCCCTA | Novel | NAA |
| ACGAGAAGGGGTCCTAGGAAT | Novel | NNNRRT |
| ACGAGGCAGGTCCCCTAGAA | Novel | NAA |
| ACGATGTATATTTGCGGGAG | Novel | NGG |
| ACGCAACCCCCACTGGCTGG | Novel | NGC |
| ACGCACGCGCTGATGGCCCA | Novel | NGA |
| ACGCACGCGCTGATGGCCCA | Novel | NNACCA |
| ACGCCAACAAGGTAGGAGCT | M82 | NGA |
| ACGCCCACCGAATGTTGCCC | Novel | NAG |
| ACGCGCTGATGGCCCATGAC | Novel | NAA |
| ACGGAGGCCTTTTGGGGTGG | Novel | NGC |
| ACGGCAGACGGAGAAGGGGA | Novel | NGA |
| ACGGGCTGAGGCCCACTCCC | Novel | NNAGGA |
| ACGGGGAGTCCGCGTAAAGA | Novel | NAG |
| ACGGGGCGCACCTCTCTTTA | Novel | NGC |
| ACGGTGGTCTCCATGCGACG | Novel | NGC |
| ACGGTTTCTCTTCCAAAAGT | Novel | NAG |
| ACGTCGCATGGAGACCACCG | Novel | NGA |
| ACTAATATGGGCCTAAAGTT | Novel | NAG |
| ACTAATGACTCTAGCTACCT | Novel | NGG |
| ACTACCGTGTGTCTTGGCCA | Novel | NAA |
| ACTACTAGGTCTCTAGATGC | Novel | NGG |
| ACTACTGTTGTTAGACGACG | Novel | NGG |
| ACTAGATGTTCTGGATAATA | Novel | NGG |
| ACTAGGAGGCTGTAGGCATAA | Novel | NNNRRT |
| ACTAGTAAACTGAGCCAGGA | Novel | NAA |
| ACTATTTACACACTCTATGG | Novel | NAG |
| ACTCATAAGGTGGGGAACTTT | Novel | NNNRRT |
| ACTCCCATAGGAATTTTCCG | Novel | NAA |
| ACTCCCTCGCCTCGCAGACG | Novel | NAG |
| ACTCCTCCAGCTTATAGACCA | Novel | NNNRRT |
| ACTCCTCCCAGTCTTTAAAC | Novel | NAA |
| ACTCCTCCCAGTCTTTAAACA | E96 | NNNRRT |
| ACTCTAAGGCTTCCCGATAC | Novel | NGA |
| ACTCTAGCTACCTGGGTGGGT | Novel | NNNRRT |
| ACTCTGCAAGATCCCAGAGT | Novel | NAG |
| ACTCTGTTGTCCTCTCCCGC | Novel | NAA |
| ACTGAAAGCCAAACAGTGGG | Novel | NGA |
| ACTGAACAAATGGCACTAGT | Novel | NAA |
| ACTGAAGGAAAGAAGTCAGA | Novel | NGG |
| ACTGGCTGGGGCTTGGTCAT | Novel | NGG |
| ACTGGGAGGAGTTGGGGGAG | Novel | NAG |
| ACTGTTGTTAGACGACGAGG | Novel | NAG |
| ACTGTTTGTTTAAAGACTGG | Novel | NAG |
| ACTGTTTGTTTAAAGACTGGG | Novel | NNGRRT |
| ACTGTTTGTTTAAAGACTGGG | Novel | NNNRRT |
| ACTTACAAGGCCTTTCTGTG | Novel | NAA |
| ACTTACAGTTAATGAGAAAA | Novel | NAA |
| ACTTCAAAGACTGTTTGTTT | Novel | NAA |
| ACTTCCAATGACATAACCCA | Novel | NAA |
| ACTTCTCTCAATTTTCTAGG | Novel | NGG |
| ACTTTCTCGCCAACTTACAA | Novel | NGC |
| AGAAAATTGGTAACAGCGGT | Novel | NAA |
| AGAAACCGTTATAGAGTATT | Novel | NGG |
| AGAAAGGCCTTGTAAGTTGG | Novel | NGA |
| AGAACATCACATCAGGATTC | Novel | NNAGGA |
| AGAAGAACCAACAAGAAGAT | Novel | NAG |
| AGAAGAACTCCCTCGCCTCG | Novel | NAG |
| AGAAGATCTCGTACTGAAGG | Novel | NAA |
| AGAAGATGAGGCATAGCAGC | Novel | NGG |
| AGAAGATTGACGATAAGGGA | Novel | NAG |
| AGAAGGGGACGAGAGAGTCC | Novel | NAA |
| AGAAGGGGTCCTAGGAATCC | Novel | NGA |
| AGAAGTCAGAAGGCAAAAAC | Novel | NAG |
| AGAATCCTCACAATACCGCA | Novel | NAG |
| AGACAAAAGAAAATTGGTAA | Novel | NAG |
| AGACAAAAGAAAATTGGTAAC | Novel | NNNRRT |
| AGACAAGAAATGTGAAACCA | Novel | NAA |
| AGACAAGAAATGTGAAACCAC | Novel | NNGRRT |
| AGACAAGAAATGTGAAACCAC | Novel | NNNRRT |
| AGACACACGGTAGTTCCCCC | Novel | NAG |
| AGACACATCCAGCGATAACC | Novel | NGG |
| AGACACCAAATACTCTATAA | Novel | NGG |
| AGACAGGTACAGTAGAAGAA | Novel | NAA |
| AGACCACCGTGAACGCCCAC | Novel | NGA |
| AGACCTGCTGCGAGCAAAAC | Novel | NAG |
| AGACCTTCGTCTGCGAGGCG | Novel | NGG |
| AGACCTTCGTCTGCGAGGCGA | Novel | NNGRRT |
| AGACCTTCGTCTGCGAGGCGA | Novel | NNNRRT |
| AGACCTTGGGCAACATTCGG | Novel | NGG |
| AGACGACGAGGCAGGTCCCC | Novel | NAG |
| AGACGGAGAAGGGGACGAGA | Novel | NAG |
| AGACGGGGAGTCCGCGTAAA | Novel | NAG |
| AGACGGGGAGTCCGCGTAAAG | Novel | NNNRRT |
| AGACTGGGAGGAGTTGGGGG | Novel | NGG |
| AGACTGGGAGGAGTTGGGGGA | Novel | NNNRRT |
| AGACTGTTTGTTTAAAGACT | Novel | NGG |
| AGAGAAGTCCACCACGAGTC | Novel | NAG |
| AGAGAGGTGCGCCCCGTGGT | Novel | NGG |
| AGAGAGTCCCAAGCGACCCC | Novel | NAG |
| AGAGATGATTAGGCAGAGGT | Novel | NAA |
| AGAGCAAACACAGCAAATCC | Novel | NGA |
| AGAGCTGAGGCGGTATCTAG | Novel | NAG |
| AGAGGAAGATGATAAAACGC | Novel | NGC |
| AGAGGCCTGTATTTCCCTGC | Novel | NGG |
| AGAGTATTTGGTGTCTTTCG | Novel | NAG |
| AGAGTCCCAAGCGACCCCGA | Novel | NAA |
| AGAGTCCCAAGCGACCCCGAG | Novel | NNGRRT |
| AGAGTCCCAAGCGACCCCGAG | Novel | NNNRRT |
| AGAGTTTGGTGAAAGGTTGT | Novel | NGA |
| AGATCCAGCATCTAGAGACC | Novel | NAG |
| AGATCCAGCATCTAGAGACCT | Novel | NNNRRT |
| AGATCTCGTACTGAAGGAAA | Novel | NAA |
| AGATCTTCTAGATACCGCCT | Novel | NAG |
| AGATCTTGTTCCCAAGAATA | Novel | NGG |
| AGATGAGAAGGCACAGACGG | Novel | NGA |
| AGATGATTAGGCAGAGGTGA | Novel | NAA |
| AGATGCTGGATCTTCCAAAT | Novel | NAA |
| AGATGCTGTACAGACTTGGCC | Novel | NNNRRT |
| AGATTAAAGGTCTTTGTACT | Novel | NGG |
| AGATTGAATACATGCATACA | Novel | NGG |
| AGATTGCAATTGATTATGCC | Novel | NGC |
| AGCAAAACCCAAAAGACCCA | Novel | NAA |
| AGCAAATCCGCCTCCTGCCT | Novel | NNACCA |
| AGCAACAGGAGGGATACATA | Novel | NAG |
| AGCAATTCTTTGCTGGGGGGA | Novel | NNNRRT |
| AGCAGTAGTCATGCAGGTTC | Novel | NGC |
| AGCCACCAGCAGGGAAATAC | M56 | NGG |
| AGCCACCCAAGGCACAGCTT | M72 | NGA |
| AGCCAGGAGAAACGGGCTGA | Novel | NGC |
| AGCCCAGGATGATGGGATGG | Novel | NAA |
| AGCCCTACGAACCACTGAAC | Novel | NAA |
| AGCCGAAAAGGTTCCACGCA | Novel | NGC |
| AGCCTGAGGGCTCCACCCCA | Novel | NAA |
| AGCCTTCAGAGCAAACACAG | Novel | NAA |
| AGCGATAACCAGGACAAGTT | Novel | NGA |
| AGCGATAACCAGGACAAGTT | Novel | NNAGGA |
| AGCGCAGGGTCCCCAATCCT | Novel | NGA |
| AGCGCCGACGGGACGTAAAC | Novel | NAA |
| AGCGCCGACGGGACGTAAAC | Novel | NNAGGA |
| AGCGGCTAGGAGTTCCGCAGT | Novel | NNGRRT |
| AGCGGCTAGGAGTTCCGCAGT | Novel | NNNRRT |
| AGCTCCAAATTCTTTATAAGG | Novel | NNNRRT |
| AGCTCTGTATCGGGAAGCCT | Novel | NAG |
| AGCTGTGCCTTGGGTGGCTT | Novel | NGG |
| AGCTTGGAGGCTTGAACAGT | Novel | NGG |
| AGGAAAGAAGTCAGAAGGCA | Novel | NAA |
| AGGAAGATGATAAAACGCCG | Novel | NAG |
| AGGAAGTTTTCTAAAACATTC | Novel | NNNRRT |
| AGGAATTTTCCGAAAGCCCA | Novel | NGA |
| AGGAATTTTCCGAAAGCCCAG | Novel | NNNRRT |
| AGGACAAGTTGGAGGACAGG | Novel | NGG |
| AGGACCCCTTCTCGTGTTAC | Novel | NGG |
| AGGACGTCCCGCGCAGGATC | Novel | NAG |
| AGGAGCAGTAAACCCTGTTC | Novel | NGA |
| AGGAGCTGGAGCATTCGGGC | Novel | NGG |
| AGGAGGCGGATTTGCTGGCA | Novel | NAG |
| AGGAGGCTGTAGGCATAAAT | E3 | NGG |
| AGGAGGTTGGTGAGTGATTG | Novel | NAG |
| AGGAGTGCGAATCCACACTC | Novel | NGA |
| AGGAGTTCCGCAGTATGGAT | E4 | NGG |
| AGGAGTTGGGGGAGGAGATT | Novel | NGA |
| AGGATCCTCAACCACCAGCA | Novel | NGG |
| AGGATCCTGGAATTAGAGGA | Novel | NAA |
| AGGATGAAGAGGAAGATGAT | Novel | NAA |
| AGGATGATGGGATGGGAATA | Novel | NAG |
| AGGATTAAAGACAGGTACAG | Novel | NAG |
| AGGATTCTTGTCAACAAGAA | Novel | NAA |
| AGGATTGGGGACCCTGCGCT | Novel | NAA |
| AGGCAAAAACGAGAGTAACTC | Novel | NNNRRT |
| AGGCAAGCAATTCTTTGCTG | M43 | NGG |
| AGGCAGGAGGCGGATTTGCT | Novel | NGC |
| AGGCAGGTCCCCTAGAAGAA | Novel | NAA |
| AGGCATAGCAGCAGGATGAA | Novel | NAG |
| AGGCCCACTTACAGTTAATG | Novel | NGA |
| AGGCCTCCGTGCGGTGGGGT | Novel | NAA |
| AGGCCTTGTAAGTTGGCGAG | Novel | NAA |
| AGGCGAGGGAGTTCTTCTTC | Novel | NAG |
| AGGCGGGTATATTATATAAG | Novel | NGA |
| AGGCTGCCTTCCTGTCTGGCG | Novel | NNNRRT |
| AGGCTTCCCGATACAGAGCT | Novel | NAG |
| AGGCTTGAACAGTAGGACAT | Novel | NAA |
| AGGGACTCAAGATGCTGTAC | Novel | NGA |
| AGGGAGAGGCAGTAGTCGGAA | Novel | NNGRRT |
| AGGGAGAGGCAGTAGTCGGAA | Novel | NNNRRT |
| AGGGATACATAGAGGTTCCT | Novel | NGA |
| AGGGCTTTCCCCCACTGTTT | Novel | NGC |
| AGGGGACCTGCCTCGTCGTC | Novel | NAA |
| AGGGGCATTTGGTGGTCTAT | Novel | NAG |
| AGGGTCCCCAATCCTCGAGA | M86 | NGA |
| AGGGTCGATGTCCATGCCCC | Novel | NAA |
| AGGGTTTAAATGTATACCCA | Novel | NAG |
| AGGGTTTACTGCTCCTGAAC | Novel | NGG |
| AGGTACAGTAGAAGAATAAAG | Novel | NNNRRT |
| AGGTAGGAGCTGGAGCATTC | Novel | NGG |
| AGGTATGTTGCCCGTTTGTCC | Novel | NNNRRT |
| AGGTATTGTTTACACAGAAA | Novel | NGC |
| AGGTCGGTCGTTGACATTGC | Novel | NGA |
| AGGTCGGTCGTTGACATTGCA | Novel | NNGRRT |
| AGGTCGGTCGTTGACATTGCA | Novel | NNNRRT |
| AGGTCTCAATCGCCGCGTCG | Novel | NAG |
| AGGTCTGGAGCAAACATTAT | Novel | NGG |
| AGGTGCGCCCCGTGGTCGGT | Novel | NGG |
| AGGTGTCCTTGTTGGGATTG | Novel | NAG |
| AGGTTCAGGTATTGTTTACA | Novel | NAG |
| AGGTTCCACGCACGCGCTGA | Novel | NGG |
| AGGTTGGGGACTGCGAATTT | Novel | NGG |
| AGGTTTGGTACAGCAACAGG | Novel | NGG |
| AGGTTTTATCCAAAGGTTAC | Novel | NAA |
| AGTAAAGTTCCCCACCTTAT | Novel | NAG |
| AGTAACTCCACAGTAGCTCC | Novel | NAA |
| AGTAATGATTAACTAGATGTT | Novel | NNGRRT |
| AGTAATGATTAACTAGATGTT | Novel | NNNRRT |
| AGTAGAAGAATAAAGACCAG | Novel | NAA |
| AGTAGCTCCAAATTCTTTAT | Novel | NAG |
| AGTAGGACATGAACAAGAGA | Novel | NGA |
| AGTAGTTTCCGGAAGTGTTG | Novel | NNAGGA |
| AGTAGTTTCCGGAAGTGTTGA | Novel | NNGRRT |
| AGTAGTTTCCGGAAGTGTTGA | Novel | NNNRRT |
| AGTCATTAGTTCCCCCCAGC | Novel | NAA |
| AGTCATTAGTTCCCCCCAGCA | Novel | NNGRRT |
| AGTCATTAGTTCCCCCCAGCA | Novel | NNNRRT |
| AGTCCAAGAGTCCTCTTATG | Novel | NAA |
| AGTCCAAGGAATACTAACAT | M78 | NGA |
| AGTCCAGAAGAACCAACAAG | Novel | NAG |
| AGTCCCAAGCGACCCCGAGA | Novel | NGG |
| AGTCCCCAACCTCCAATCAC | Novel | NNACCA |
| AGTCCCGATAATGTTTGCTC | Novel | NAG |
| AGTCCGCGTAAAGAGAGGTG | Novel | NGC |
| AGTCCTCTTATGTAAGACCT | Novel | NGG |
| AGTCTTTAAACAAACAGTCTT | Novel | NNNRRT |
| AGTCTTTGAAGTATGCCTCA | Novel | NGG |
| AGTGAAAGCCTGCTTAGATT | Novel | NAA |
| AGTGATTGGAGGTTGGGGAC | Novel | NGC |
| AGTGATTGGAGGTTGGGGACT | Novel | NNGRRT |
| AGTGATTGGAGGTTGGGGACT | Novel | NNNRRT |
| AGTGCACACGGTCCGGCAGA | Novel | NGA |
| AGTGCGAATCCACACTCCGA | Novel | NAG |
| AGTGTCTAGTTTGGAAGTAA | Novel | NGA |
| AGTGTGGATTCGCACTCCTC | Novel | NAG |
| AGTGTTGATAGGATAGGGGCA | Novel | NNNRRT |
| AGTTAATGAGAAAAGAAGAT | Novel | NGC |
| AGTTATGGGTCCTTGCCACA | Novel | NGA |
| AGTTATGTCAACACTAATAT | Novel | NGG |
| AGTTCCCCACCTTATGAGTC | Novel | NAA |
| AGTTCCCCACCTTATGAGTC | Novel | NNAGGA |
| AGTTCCCCCCAGCAAAGAAT | Novel | NGC |
| AGTTCCCCCTAGAAAATTGA | Novel | NAG |
| AGTTCCCCCTAGAAAATTGAG | Novel | NNNRRT |
| AGTTCCGCAGTATGGATCGG | Novel | NAG |
| AGTTCTTCTTCTAGGGGACC | Novel | NGC |
| AGTTGCATGGTGCTGGTGCG | Novel | NAG |
| AGTTGGCAGCACAGCCTAGC | Novel | NGC |
| AGTTTCCGGAAGTGTTGATA | Novel | NGA |
| ATAAAGAATTTGGAGCTACTG | Novel | NNGRRT |
| ATAAAGAATTTGGAGCTACTG | Novel | NNNRRT |
| ATAAATTGGTCTGCGCACCA | Novel | NNACCA |
| ATAACCACATTGTGTAAATG | Novel | NGG |
| ATAACTCTGTTGTCCTCTCC | Novel | NGC |
| ATAACTCTGTTGTCCTCTCCC | Novel | NNNRRT |
| ATAACTGAAAGCCAAACAGT | Novel | NGG |
| ATAACTGACTACTAGGTCTC | Novel | NAG |
| ATAAGAGAGAAACAACACAT | Novel | NGC |
| ATAAGGGAGAGGCAGTAGTC | Novel | NGA |
| ATAATATACCCGCCTTCCAT | Novel | NGA |
| ATACAGGTGCAATTTCCGTC | Novel | NGA |
| ATACAGGTGCAATTTCCGTCC | Novel | NNNRRT |
| ATACATAGAGGTTCCTTGAG | Novel | NAG |
| ATACATAGAGGTTCCTTGAGC | Novel | NNNRRT |
| ATACATGCATACAAGGGCAT | Novel | NAA |
| ATACCGCCTCAGCTCTGTAT | Novel | NGG |
| ATACGATGTATATTTGCGGG | Novel | NGA |
| ATACGATGTATATTTGCGGG | Novel | NNAGGA |
| ATACTAACATTGAGGTTCCC | Novel | NAG |
| ATACTACAAACTTTGCCAGC | Novel | NAA |
| ATAGAGTATTTGGTGTCTTT | Novel | NGG |
| ATAGCAGCAGGATGAAGAGG | Novel | NAG |
| ATAGCGCCTCATTTTGTGGG | Novel | NNACCA |
| ATAGGAATTTTCCGAAAGCC | Novel | NAG |
| ATAGTCCAGAAGAACCAACA | Novel | NGA |
| ATAGTCCAGAAGAACCAACAA | Novel | NNNRRT |
| ATATAATATACCCGCCTTCCA | Novel | NNGRRT |
| ATATAATATACCCGCCTTCCA | Novel | NNNRRT |
| ATATACATCGTATCCATGGC | Novel | NGC |
| ATATGGATGATGTGGTATTG | Novel | NGG |
| ATATGGGCCTAAAGTTCAGG | Novel | NAA |
| ATATGGTGACCCACAAAATG | Novel | NGG |
| ATATTTGCGGGAGAGGACAA | Novel | NAG |
| ATATTTGGTAACCTTTGGAT | Novel | NAA |
| ATCAATAGGCCTGTTAATAG | Novel | NAA |
| ATCCAAAGGTTACCAAATAT | Novel | NNACCA |
| ATCCAACTGGTGGTCGGGAA | Novel | NGA |
| ATCCAATGGTAAATATTTGG | Novel | NAA |
| ATCCACACTCCGAAAGACAC | Novel | NAA |
| ATCCAGTTGGCAGCACAGCC | Novel | NAG |
| ATCCATACTGCGGAACTCCT | Novel | NGC |
| ATCCCAGAGGATTGCTGGTG | Novel | NAA |
| ATCCCAGAGGATTGCTGGTGG | Novel | NNNRRT |
| ATCCCATCATCCTGGGCTTT | Novel | NGG |
| ATCCCTCCTGTTGCTGTACC | Novel | NAA |
| ATCCTCGAGAAGATTGACGA | Novel | NAA |
| ATCCTGCGGACGACCCTTCT | Novel | NGG |
| ATCGACCCTTATAAAGAATT | Novel | NGG |
| ATCTAGAAGATCTCGTACTG | Novel | NAG |
| ATCTAGTTAATCATTACTTC | Novel | NAA |
| ATCTCTTTGTTTTGTTAGGGT | Novel | NNNRRT |
| ATCTTCCAAATTAACACCCAC | Novel | NNNRRT |
| ATCTTCTGCGACGCGGCGAT | Novel | NGA |
| ATCTTCTTGTTGGTTCTTCT | Novel | NGA |
| ATCTTGCAGAGTTTGGTGAA | Novel | NGG |
| ATCTTTCCACCAGCAATCCTC | Novel | NNGRRT |
| ATCTTTCCACCAGCAATCCTC | Novel | NNNRRT |
| ATGAACAAGAGATGATTAGG | Novel | NAG |
| ATGAACAAGAGATGATTAGGC | Novel | NNNRRT |
| ATGACATAACCCATAAAATT | Novel | NAG |
| ATGACCAAGCCCCAGCCAGT | Novel | NGG |
| ATGACTCTAGCTACCTGGGT | Novel | NGG |
| ATGAGGATTAAAGACAGGTA | Novel | NAG |
| ATGAGGCATAGCAGCAGGAT | Novel | NAA |
| ATGAGTGTTTCTCAAAGGTG | Novel | NAG |
| ATGATGGGATGGGAATACAGG | Novel | NNNRRT |
| ATGATGTGGTATTGGGGGCC | Novel | NAG |
| ATGATGTGTTCTTGTGGCAA | Novel | NGA |
| ATGATTAGGCAGAGGTGAAA | Novel | NAG |
| ATGCATACAAGGGCATTAAC | Novel | NNAGGA |
| ATGCATACAAGGGCATTAACG | Novel | NNGRRT |
| ATGCATACAAGGGCATTAACG | Novel | NNNRRT |
| ATGCATGTATTCAATCTAAG | Novel | NAG |
| ATGCCTACAGCCTCCTAGTA | Novel | NAA |
| ATGCCTGCTAGGTTTTATCC | Novel | NAA |
| ATGCGACGTGCAGAGGTGAAG | Novel | NNNRRT |
| ATGCTGTAGATCTTGTTCCC | Novel | NAG |
| ATGGACATCGACCCTTATAA | Novel | NGA |
| ATGGATACGATGTATATTTG | Novel | NGG |
| ATGGATCGGCAGAGGAGCCG | Novel | NAA |
| ATGGATCGGCAGAGGAGCCGA | Novel | NNNRRT |
| ATGGATGATGTGGTATTGGG | Novel | NGC |
| ATGGCACTAGTAAACTGAGC | Novel | NAG |
| ATGGCCCATGACCAAGCCCC | Novel | NGC |
| ATGGCCCATGACCAAGCCCCA | Novel | NNNRRT |
| ATGGGATGGGAATACAGGTG | Novel | NAA |
| ATGGGCCATCAGCGCGTGCG | Novel | NGG |
| ATGGGGCAGAATCTTTCCAC | Novel | NAG |
| ATGGGGCAGAATCTTTCCACC | Novel | NNNRRT |
| ATGGGGCAGCAAAACCCAAA | Novel | NGA |
| ATGGTAAATATTTGGTAACCT | Novel | NNGRRT |
| ATGGTAAATATTTGGTAACCT | Novel | NNNRRT |
| ATGGTCCCGTGCTGGTGGTT | Novel | NAG |
| ATGGTGAGGTGAACAATGCT | Novel | NAG |
| ATGTATACCCAAAGACAAAA | Novel | NAA |
| ATGTCAACACTAATATGGGCC | Novel | NNNRRT |
| ATGTCAACGACCGACCTTGA | Novel | NGC |
| ATGTGATGTTCTCCATGTTC | Novel | NGC |
| ATGTGGTTATCCTGCGTTAA | Novel | NGC |
| ATGTTCTCCATGTTCAGCGC | Novel | NGG |
| ATGTTCTGGATAATAAGGTT | Novel | NAA |
| ATGTTGCCCAAGGTCTTACA | Novel | NAA |
| ATTAAAGACAGGTACAGTAG | Novel | NAG |
| ATTAAAGGTCTTTGTACTAG | Novel | NAG |
| ATTAACACCCACCCAGGTAGC | Novel | NNGRRT |
| ATTAACACCCACCCAGGTAGC | Novel | NNNRRT |
| ATTAACAGGCCTATTGATTG | Novel | NAA |
| ATTAACTGTAAGTGGGCCTA | Novel | NAA |
| ATTCAGCGCCGACGGGACGT | Novel | NAA |
| ATTCCAGGATCCTCAACCAC | Novel | NAG |
| ATTCCCATCCCATCATCCTG | Novel | NGC |
| ATTCCTATGGGAGTGGGCCT | Novel | NAG |
| ATTCCTTGGACTCATAAGGT | Novel | NGG |
| ATTCGCACTCCTCCAGCTTA | Novel | NAG |
| ATTCTTGGGAACAAGATCTA | Novel | NAG |
| ATTCTTTCCCGACCACCAGT | Novel | NGG |
| ATTGAATACATGCATACAAG | Novel | NGC |
| ATTGAGACCTTCGTCTGCGA | Novel | NGC |
| ATTGAGATCTTCTGCGACGC | Novel | NGC |
| ATTGGGACTTCAATCCCAAC | Novel | NAG |
| ATTGGGGGCCAAGTCTGTAC | Novel | NGC |
| ATTGGTAACAGCGGTAAAAA | Novel | NGG |
| ATTGTGAGGATTCTTGTCAA | Novel | NAA |
| ATTGTGGGTCTTTTGGGTTT | Novel | NGC |
| ATTGTGTAAATGGGGCAGCA | Novel | NAA |
| ATTTAAACCCTAACAAAACA | Novel | NAG |
| ATTTAAACCCTAACAAAACAA | Novel | NNNRRT |
| ATTTACACCAAGACATTATC | Novel | NAA |
| ATTTACACCAAGACATTATCA | M16 | NNNRRT |
| ATTTCTTGTCTCACTTTTGG | Novel | NAG |
| ATTTGCGGGAGAGGACAACA | Novel | NAG |
| ATTTGCTGTGTTTGCTCTGA | Novel | NGG |
| ATTTGGAAGATCCAGCATCT | Novel | NGA |
| ATTTGGTGGTCTATAAGCTG | Novel | NAG |
| ATTTGGTGGTCTATAAGCTGG | Novel | NNGRRT |
| ATTTGGTGGTCTATAAGCTGG | Novel | NNNRRT |
| ATTTGGTGTCTTTCGGAGTG | Novel | NGG |
| ATTTGTTCAGTGGTTCGTAG | Novel | NGC |
| ATTTTATGGGTTATGTCATT | Novel | NGA |
| ATTTTGGCCAAGACACACGG | Novel | NAG |
| ATTTTTTGATAATGTCTTGGT | Novel | NNNRRT |
| CAAAAAATCAAAGAATGTTT | Novel | NAG |
| CAAAAAATGTGAACAGTTTG | Novel | NAG |
| CAAAAACGAGAGTAACTCCA | Novel | NAG |
| CAAAAGAAAATTGGTAACAG | Novel | NGG |
| CAAAAGGCCTCCGTGCGGTG | Novel | NGG |
| CAAAAGTGAGACAAGAAATG | Novel | NGA |
| CAAAATTCGCAGTCCCCAACC | Novel | NNNRRT |
| CAAACACAGCAAATCCAGAT | Novel | NGG |
| CAAACACTTGGCACAGACCT | Novel | NGC |
| CAAAGAATTGCTTGCCTGAG | Novel | NGC |
| CAAAGACAAAAGAAAATTGG | Novel | NAA |
| CAAAGGACGTCCCGCGCAGGA | Novel | NNNRRT |
| CAAAGGTGGAGACAGCGGGG | Novel | NAG |
| CAAAGTTTGTAGTATGCCCT | Novel | NAG |
| CAAATATACATCGTATCCAT | Novel | NGC |
| CAAATATTTACCATTGGATA | Novel | NGG |
| CAAATGGCACTAGTAAACTG | Novel | NGC |
| CAAATTAACACCCACCCAGG | Novel | NAG |
| CAACACATAGCGCCTCATTTT | Novel | NNGRRT |
| CAACACATAGCGCCTCATTTT | Novel | NNNRRT |
| CAACATACCTTGATAGTCCA | Novel | NAA |
| CAACATTCGGTGGGCGTTCA | Novel | NGG |
| CAACATTCGGTGGGCGTTCAC | Novel | NNNRRT |
| CAACCACCAGCACGGGACCA | Novel | NGC |
| CAACCTTTCACCAAACTCTG | Novel | NAA |
| CAACGAATTGTGGGTCTTTT | M62 | NGG |
| CAACTTGTCCTGGTTATCGC | Novel | NGG |
| CAAGAAATGTGAAACCACAA | Novel | NAG |
| CAAGAAGATGAGGCATAGCA | Novel | NNAGGA |
| CAAGAAGATGAGGCATAGCAG | Novel | NNGRRT |
| CAAGAAGATGAGGCATAGCAG | Novel | NNNRRT |
| CAAGAATATGGTGACCCACA | Novel | NAA |
| CAAGAATCCTCACAATACCG | Novel | NAG |
| CAAGACATTATCAAAAAATG | Novel | NGA |
| CAAGAGTTGCCTGAACTTTA | Novel | NGC |
| CAAGATCTACAGCATGGGGC | Novel | NGA |
| CAAGCAATTCTTTGCTGGGG | Novel | NGA |
| CAAGCCTCCAAGCTGTGCCT | E5 | NGG |
| CAAGGACCCATAACTTCCAA | Novel | NGA |
| CAAGGCACAGCTTGGAGGCT | E6 | NGA |
| CAAGTTGGAGGACAGGAGGT | Novel | NGG |
| CAATACCGCAGAGTCTAGACT | M1 | NNNRRT |
| CAATCAATAGGCCTGTTAAT | Novel | NGG |
| CAATCAATAGGCCTGTTAATA | Novel | NNNRRT |
| CAATCCCAACAAGGACACCT | Novel | NGC |
| CAATCGCCAGACAGGAAGGC | Novel | NGC |
| CAATCTGGATTTGCTGTGTT | Novel | NGC |
| CAATGCTCAGGAGACTCTAA | Novel | NGC |
| CAATGTCAACGACCGACCTT | Novel | NAG |
| CAATTCGTTGACATACTTTC | Novel | NAA |
| CACAACCTTTCACCAAACTC | Novel | NGC |
| CACAAGAACACATCATACAA | Novel | NAA |
| CACAAGAGTTGCCTGAACTT | Novel | NAG |
| CACACGGTAGTTCCCCCTAG | Novel | NAA |
| CACACGGTCCGGCAGATGAG | Novel | NAG |
| CACAGAAAGGCCTTGTAAGT | Novel | NGG |
| CACAGACCTGGCCGTTGCCG | Novel | NGC |
| CACAGACGGGGAGTCCGCGT | Novel | NAA |
| CACATAGCGCCTCATTTTGT | Novel | NGG |
| CACATCATACAAAAAATCAA | Novel | NGA |
| CACATCATCCATATAACTGA | Novel | NAG |
| CACATTTCTTGTCTCACTTT | Novel | NGG |
| CACATTTTTTGATAATGTCT | Novel | NGG |
| CACCAAACTCTGCAAGATCC | Novel | NAG |
| CACCAATCGCCAGACAGGAA | Novel | NGC |
| CACCACGAGTCTAGACTCTG | M36 | NGG |
| CACCAGCACGGGACCATGCC | Novel | NAA |
| CACCAGTTGGATCCAGCCTT | Novel | NAG |
| CACCATACTGCACTCAGGCA | Novel | NGC |
| CACCCAAGGCACAGCTTGGA | Novel | NGC |
| CACCCCAAAAGGCCTCCGTG | Novel | NGG |
| CACCGCACGGAGGCCTTTTG | Novel | NGG |
| CACCTCACCATACTGCACTC | Novel | NGG |
| CACCTCTGCACGTCGCATGG | Novel | NGA |
| CACCTCTGCACGTCGCATGG | Novel | NNACCA |
| CACCTGGCCAGACGCCAACA | Novel | NGG |
| CACGGAGGCCTTTTGGGGTG | Novel | NAG |
| CACGGTCCGGCAGATGAGAA | Novel | NGC |
| CACTAGTAAACTGAGCCAGG | Novel | NGA |
| CACTATTTACACACTCTATG | Novel | NAA |
| CACTCAGGCAAGCAATTCTT | Novel | NGC |
| CACTCCCATAGGAATTTTCC | Novel | NAA |
| CACTGAACAAATGGCACTAG | Novel | NAA |
| CACTGGCTGGGGCTTGGTCA | Novel | NGG |
| CACTGTTTGGCTTTCAGTTAT | Novel | NNGRRT |
| CACTGTTTGGCTTTCAGTTAT | Novel | NNNRRT |
| CACTTACAGTTAATGAGAAA | Novel | NGA |
| CACTTACAGTTAATGAGAAAA | Novel | NNNRRT |
| CACTTTCTCGCCAACTTACA | Novel | NGG |
| CAGAAGAACCAACAAGAAGA | Novel | NGA |
| CAGACACATCCAGCGATAAC | Novel | NAG |
| CAGACCTGCTGCGAGCAAAA | Novel | NAA |
| CAGACCTGGCCGTTGCCGGG | Novel | NAA |
| CAGACGAAGGTCTCAATCGC | Novel | NGC |
| CAGACGGAGAAGGGGACGAG | Novel | NGA |
| CAGACGGGGAGTCCGCGTAA | Novel | NGA |
| CAGAGCAAACACAGCAAATC | Novel | NAG |
| CAGAGCTGAGGCGGTATCTA | Novel | NAA |
| CAGAGTTTGGTGAAAGGTTG | Novel | NGG |
| CAGATGAGAAGGCACAGACG | Novel | NGG |
| CAGCAAAGAATTGCTTGCCT | Novel | NAG |
| CAGCAACAGGAGGGATACAT | Novel | NGA |
| CAGCACAGCCTAGCAGCCAT | Novel | NGA |
| CAGCAGGATGAAGAGGAAGA | Novel | NGA |
| CAGCATGGGGCAGAATCTTT | Novel | NNACCA |
| CAGCCAGTGGGGGTTGCGTC | Novel | NGC |
| CAGCCTAGCAGCCATGGATA | Novel | NGA |
| CAGCCTCCTAGTACAAAGACC | Novel | NNNRRT |
| CAGCGATAACCAGGACAAGT | Novel | NGG |
| CAGCGCCGACGGGACGTAAA | Novel | NAA |
| CAGCGGTAAAAAGGGACTCA | Novel | NGA |
| CAGCTCTGTATCGGGAAGCCT | Novel | NNGRRT |
| CAGCTCTGTATCGGGAAGCCT | Novel | NNNRRT |
| CAGCTTGGAGGCTTGAACAG | Novel | NAG |
| CAGGACAAGTTGGAGGACAG | Novel | NAG |
| CAGGAGACTCTAAGGCTTCC | Novel | NGA |
| CAGGAGGCGGATTTGCTGGC | Novel | NAA |
| CAGGAGGTTGGTGAGTGATT | Novel | NGA |
| CAGGATCCAGTTGGCAGCAC | Novel | NGC |
| CAGGATGAAGAGGAAGATGA | Novel | NAA |
| CAGGCAAGCAATTCTTTGCT | M42 | NGG |
| CAGGGTCCCCAATCCTCGAG | Novel | NAG |
| CAGGTACAGTAGAAGAATAA | Novel | NGA |
| CAGGTACAGTAGAAGAATAA | Novel | NNACCA |
| CAGGTATTGTTTACACAGAA | Novel | NGG |
| CAGGTGCAATTTCCGTCCGA | Novel | NGG |
| CAGGTGTCCTTGTTGGGATT | Novel | NAA |
| CAGTAAAGTTCCCCACCTTA | Novel | NGA |
| CAGTAGAAGAATAAAGACCAG | Novel | NNNRRT |
| CAGTAGCTCCAAATTCTTTA | Novel | NAA |
| CAGTAGCTCCAAATTCTTTAT | Novel | NNGRRT |
| CAGTAGCTCCAAATTCTTTAT | Novel | NNNRRT |
| CAGTATGGTGAGGTGAACAA | Novel | NGC |
| CAGTCTTTGAAGTATGCCTC | Novel | NAG |
| CAGTTAATGAGAAAAGAAGAT | Novel | NNNRRT |
| CAGTTATGTCAACACTAATA | Novel | NGG |
| CAGTTGGATCCAGCCTTCAG | Novel | NGC |
| CAGTTGGCAGCACAGCCTAG | Novel | NAG |
| CAGTTTGTAGGCCCACTTACA | Novel | NNNRRT |
| CATAAATTGGTCTGCGCACC | Novel | NGC |
| CATAACCCATAAAATTCAGA | Novel | NAG |
| CATAAGGTGGGGAACTTTAC | Novel | NGG |
| CATACAAGGGCATTAACGCA | Novel | NGA |
| CATACCTTGATAGTCCAGAA | Novel | NAA |
| CATACCTTGATAGTCCAGAA | Novel | NNACCA |
| CATACTACAAACTTTGCCAG | Novel | NAA |
| CATACTGCGGAACTCCTAGC | Novel | NGC |
| CATAGAGGTTCCTTGAGCAG | Novel | NAG |
| CATAGCAGCAGGATGAAGAG | Novel | NAA |
| CATAGGAATTTTCCGAAAGC | Novel | NNAGGA |
| CATAGGAATTTTCCGAAAGCC | Novel | NNGRRT |
| CATAGGAATTTTCCGAAAGCC | Novel | NNNRRT |
| CATATTAGTGTTGACATAAC | Novel | NGA |
| CATCATCCTGGGCTTTCGGA | Novel | NAA |
| CATCCAGCGATAACCAGGAC | Novel | NAG |
| CATCCATATAACTGAAAGCC | Novel | NAA |
| CATCCATATAACTGAAAGCCA | M6 | NNNRRT |
| CATCGTATCCATGGCTGCTA | Novel | NGC |
| CATCTTCCTCTTCATCCTGC | Novel | NGC |
| CATCTTCTTGTTGGTTCTTC | Novel | NGG |
| CATCTTGAGTCCCTTTTTAC | Novel | NGC |
| CATGACCAAGCCCCAGCCAG | Novel | NGG |
| CATGACCAAGCCCCAGCCAGT | Novel | NNGRRT |
| CATGACCAAGCCCCAGCCAGT | Novel | NNNRRT |
| CATGCAACTTTTTCACCTCTG | M8 | NNNRRT |
| CATGCATACAAGGGCATTAA | Novel | NGC |
| CATGCCCCAAAGCCACCCAA | Novel | NGC |
| CATGCGACGTGCAGAGGTGA | Novel | NGC |
| CATGCTGTAGATCTTGTTCC | Novel | NAA |
| CATGCTGTAGATCTTGTTCCC | Novel | NNGRRT |
| CATGCTGTAGATCTTGTTCCC | Novel | NNNRRT |
| CATGGACATCGACCCTTATA | Novel | NAG |
| CATGGTCCCGTGCTGGTGGT | Novel | NGA |
| CATGGTCCCGTGCTGGTGGT | Novel | NNAGGA |
| CATGGTCCCGTGCTGGTGGTT | Novel | NNGRRT |
| CATGGTCCCGTGCTGGTGGTT | Novel | NNNRRT |
| CATGGTGCTGGTGCGCAGAC | Novel | NAA |
| CATGTTCAGCGCAGGGTCCC | Novel | NAA |
| CATTAGTTCCCCCCAGCAAA | Novel | NAA |
| CATTCGGTGGGCGTTCACGG | Novel | NGG |
| CATTGAGGTTCCCGAGATTG | Novel | NGA |
| CATTGGAAAACACCATCTTTT | Novel | NNNRRT |
| CATTGGAAGTTATGGGTCCT | Novel | NGC |
| CATTGTGTAAATGGGGCAGC | Novel | NAA |
| CATTGTTCACCTCACCATAC | Novel | NGC |
| CATTTAAACCCTAACAAAAC | Novel | NAA |
| CATTTACACCAAGACATTAT | Novel | NAA |
| CATTTCTTGTCTCACTTTTG | Novel | NAA |
| CATTTGGTGGTCTATAAGCT | Novel | NGA |
| CATTTGGTGGTCTATAAGCT | Novel | NNAGGA |
| CATTTGTTCAGTGGTTCGTA | Novel | NGG |
| CCAAAAGACCCACAATTCGT | Novel | NGA |
| CCAAAAGGCCTCCGTGCGGT | Novel | NGG |
| CCAAACCTTCGGACGGAAAT | Novel | NGC |
| CCAAACTCTGCAAGATCCCA | Novel | NAG |
| CCAAATATTTACCATTGGAT | Novel | NAG |
| CCAACAAGAAGATGAGGCAT | Novel | NGC |
| CCAAGAATATGGTGACCCAC | Novel | NAA |
| CCAAGTCTGTACAGCATCTT | Novel | NAG |
| CCAATACCACATCATCCATA | Novel | NAA |
| CCAATCAATAGGCCTGTTAA | Novel | NAG |
| CCAATCCTCGAGAAGATTGA | M84 | NGA |
| CCAATCGCCAGACAGGAAGG | Novel | NAG |
| CCAATGAGGATTAAAGACAGG | Novel | NNNRRT |
| CCACAAGAACACATCATACA | Novel | NAA |
| CCACAATTCGTTGACATACTT | Novel | NNNRRT |
| CCACATCATCCATATAACTG | Novel | NAA |
| CCACATTGTGTAAATGGGGC | Novel | NGC |
| CCACCAATCGCCAGACAGGA | M55 | NGG |
| CCACCAGCACGGGACCATGC | M90 | NGA |
| CCACCCAAGGCACAGCTTGG | M38 | NGG |
| CCACCGCACGGAGGCCTTTT | Novel | NGG |
| CCACTCCCATAGGAATTTTC | M69 | NGA |
| CCACTGCATGGCCTGAGGAT | Novel | NAG |
| CCACTTACAGTTAATGAGAA | Novel | NAG |
| CCAGACGCCAACAAGGTAGG | Novel | NGC |
| CCAGAGGATTGCTGGTGGAA | Novel | NGA |
| CCAGATTGGGACTTCAATCC | Novel | NAA |
| CCAGCAAAGAATTGCTTGCC | M73 | NGA |
| CCAGCATCTAGAGACCTAGTA | Novel | NNNRRT |
| CCAGCCAGTGGGGGTTGCGT | Novel | NAG |
| CCAGCCTTCAGAGCAAACAC | Novel | NGC |
| CCAGCCTTCAGAGCAAACACA | M22 | NNNRRT |
| CCAGCTTATAGACCACCAAA | Novel | NGC |
| CCAGGACAAGTTGGAGGACA | M88 | NGA |
| CCAGGATGATGGGATGGGAAT | Novel | NNNRRT |
| CCAGGTCTGTGCCAAGTGTT | Novel | NGC |
| CCAGGTGTCCTTGTTGGGAT | Novel | NGA |
| CCAGTGGGGGTTGCGTCAGC | Novel | NAA |
| CCAGTTGGATCCAGCCTTCA | Novel | NAG |
| CCATACTGCACTCAGGCAAG | Novel | NAA |
| CCATAGAGTGTGTAAATAGTG | Novel | NNNRRT |
| CCATATAACTGAAAGCCAAA | Novel | NAG |
| CCATCATCCTGGGCTTTCGG | Novel | NAA |
| CCATGCAACTTTTTCACCTC | Novel | NGC |
| CCATGCCCCAAAGCCACCCA | M40 | NGG |
| CCATGCGACGTGCAGAGGTG | Novel | NAG |
| CCATGGATACGATGTATATT | Novel | NGC |
| CCATGGCTGCTAGGCTGTGC | Novel | NGC |
| CCATTTGTTCAGTGGTTCGT | Novel | NGG |
| CCCAAAAGGCCTCCGTGCGG | Novel | NGG |
| CCCAAAGCCACCCAAGGCAC | Novel | NGC |
| CCCAACAAGGACACCTGGCC | M81 | NGA |
| CCCAACCTCCAATCACTCAC | Novel | NAA |
| CCCAACTCCTCCCAGTCTTT | Novel | NAA |
| CCCAAGAATATGGTGACCCA | Novel | NAA |
| CCCACCCAGGTAGCTAGAGTC | M11 | NNNRRT |
| CCCACCGCACGGAGGCCTTT | Novel | NGG |
| CCCACTTACAGTTAATGAGA | Novel | NAA |
| CCCAGAGGATTGCTGGTGGA | Novel | NAG |
| CCCATCATCCTGGGCTTTCG | Novel | NAA |
| CCCATCTCTTTGTTTTGTTA | M59 | NGG |
| CCCCAAAAGGCCTCCGTGCGG | Novel | NNGRRT |
| CCCCAAAAGGCCTCCGTGCGG | Novel | NNNRRT |
| CCCCAAAGCCACCCAAGGCA | Novel | NAG |
| CCCCAACTCCTCCCAGTCTT | Novel | NAA |
| CCCCACCGCACGGAGGCCTTT | Novel | NNGRRT |
| CCCCACCGCACGGAGGCCTTT | Novel | NNNRRT |
| CCCCACCTTATGAGTCCAAG | Novel | NAA |
| CCCCAGCAAAGAATTGCTTGC | M10 | NNGRRT |
| CCCCAGCAAAGAATTGCTTGC | Novel | NNNRRT |
| CCCCATCTCTTTGTTTTGTT | M60 | NGG |
| CCCCCCAGCAAAGAATTGCT | Novel | NGC |
| CCCCGAGAAGGGTCGTCCGC | Novel | NGG |
| CCCCGCCTGTAACACGAGAA | Novel | NGG |
| CCCCGCTGTCTCCACCTTTG | Novel | NGA |
| CCCCGTTGCCCGGCAACGGC | Novel | NAG |
| CCCCTTCTCGTGTTACAGGC | Novel | NGG |
| CCCGACCACCAGTTGGATCC | Novel | NGC |
| CCCGAGAAGGGTCGTCCGCA | Novel | NGA |
| CCCGAGATTGAGATCTTCTG | Novel | NGA |
| CCCGCCTGTAACACGAGAAG | Novel | NGG |
| CCCGCCTTCCATAGAGTGTG | Novel | NAA |
| CCCGCGCAGGATCCAGTTGG | Novel | NAG |
| CCCGCTGTCTCCACCTTTGA | Novel | NAA |
| CCCGGCAACGGCCAGGTCTG | Novel | NGC |
| CCCGTCGGCGCTGAATCCTG | Novel | NGG |
| CCCGTCTGTGCCTTCTCATC | Novel | NGC |
| CCCGTGGTCGGTCGGAACGG | Novel | NAG |
| CCCGTTGCCCGGCAACGGCC | Novel | NGG |
| CCCGTTTGTCCTCTAATTCC | Novel | NGG |
| CCCTAACAAAACAAAGAGAT | Novel | NGG |
| CCCTAGAAGAAGAACTCCCT | Novel | NGC |
| CCCTATCCTATCAACACTTC | Novel | NGG |
| CCCTGCTGGTGGCTCCAGTT | Novel | NAG |
| CCCTTATCGTCAATCTTCTC | Novel | NAG |
| CCCTTCTCCGTCTGCCGTTC | Novel | NGA |
| CCCTTCTCGTGTTACAGGCG | Novel | NGG |
| CCGAAAAGGTTCCACGCACG | Novel | NGC |
| CCGAAAAGGTTCCACGCACGC | Novel | NNNRRT |
| CCGAAGGTTTGGTACAGCAA | Novel | NAG |
| CCGACCTTGAGGCATACTTC | Novel | NAA |
| CCGCAGGATTCAGCGCCGAC | Novel | NGG |
| CCGCAGTATGGATCGGCAGA | E7 | NGA |
| CCGCCTCAGCTCTGTATCGG | Novel | NAA |
| CCGCCTTCCATAGAGTGTGT | Novel | NAA |
| CCGCCTTCCATAGAGTGTGTA | M19 | NNNRRT |
| CCGCGCAGGATCCAGTTGGC | Novel | NGC |
| CCGCTGTCTCCACCTTTGAG | Novel | NAA |
| CCGCTTGTTTTGCTCGCAGC | Novel | NGG |
| CCGGAAGTGTTGATAGGATA | Novel | NGG |
| CCGGCAACGGCCAGGTCTGTG | M32 | NNNRRT |
| CCGTCGGCGCTGAATCCTGC | Novel | NGA |
| CCGTGCGGTGGGGTGAAACC | Novel | NAG |
| CCGTGGTCGGTCGGAACGGC | Novel | NGA |
| CCGTGTGTCTTGGCCAAAATT | Novel | NNNRRT |
| CCGTTGCCGGGCAACGGGGT | Novel | NAA |
| CCGTTTCTCCTGGCTCAGTTT | Novel | NNNRRT |
| CCGTTTGTCCTCTAATTCCA | Novel | NGA |
| CCTAACAAAACAAAGAGATG | Novel | NGG |
| CCTAATATACATTTACACCA | Novel | NGA |
| CCTACAGCCTCCTAGTACAA | Novel | NGA |
| CCTACGAACCACTGAACAAA | Novel | NGG |
| CCTAGAAAATTGAGAGAAGT | Novel | NNACCA |
| CCTATCCTATCAACACTTCC | Novel | NGA |
| CCTCAATGTTAGTATTCCTT | Novel | NGA |
| CCTCACAATACCGCAGAGTC | Novel | NAG |
| CCTCCAAGCTGTGCCTTGGG | Novel | NGG |
| CCTCCACCAATCGCCAGACA | M83 | NGA |
| CCTCCAGCTTATAGACCACC | Novel | NAA |
| CCTCCCAGTCTTTAAACAAA | Novel | NAG |
| CCTCCTAGTACAAAGACCTT | Novel | NAA |
| CCTCGAGAAGATTGACGATA | Novel | NGG |
| CCTCGCCTCGCAGACGAAGGT | Novel | NNNRRT |
| CCTCTATGTATCCCTCCTGT | Novel | NGC |
| CCTCTCACTCTGGGATCTTG | Novel | NAG |
| CCTCTGCCGATCCATACTGC | E8 | NGA |
| CCTCTGGGATTCTTTCCCGA | Novel | NNACCA |
| CCTGAACTGGAGCCACCAGC | Novel | NGG |
| CCTGAACTTTAGGCCCATAT | Novel | NAG |
| CCTGAGGATGAGTGTTTCTC | Novel | NAA |
| CCTGAGGGCTCCACCCCAAA | Novel | NGG |
| CCTGCATGACTACTGCTCAA | Novel | NGA |
| CCTGCCTCGTCGTCTAACAA | Novel | NAG |
| CCTGCCTCGTCGTCTAACAAC | Novel | NNNRRT |
| CCTGCGGACGACCCTTCTCG | Novel | NGG |
| CCTGCGTTAATGCCCTTGTA | Novel | NGC |
| CCTGCTGCGAGCAAAACAAG | Novel | NGG |
| CCTGCTGGTGGCTCCAGTTC | Novel | NGG |
| CCTGGAATTAGAGGACAAAC | Novel | NGG |
| CCTGGCCAGACGCCAACAAG | Novel | NNAGGA |
| CCTGGCCGTTGCCGGGCAAC | Novel | NGG |
| CCTGGGTGGGTGTTAATTTG | Novel | NAA |
| CCTGTCTGGCGATTGGTGGA | Novel | NGC |
| CCTGTCTTTAATCCTCATTG | Novel | NAA |
| CCTGTTAATAGGAAGTTTTC | Novel | NAA |
| CCTTATCGTCAATCTTCTCG | Novel | NGG |
| CCTTATTATCCAGAACATCTA | Novel | NNNRRT |
| CCTTCAGTACGAGATCTTCT | Novel | NGA |
| CCTTCCATAGAGTGTGTAAA | Novel | NAG |
| CCTTCCTGTCTGGCGATTGG | Novel | NGG |
| CCTTCGTCTGCGAGGCGAGG | Novel | NAG |
| CCTTGATAGTCCAGAAGAAC | Novel | NAA |
| CCTTGGACTCATAAGGTGGG | Novel | NAA |
| CCTTGGGTGGCTTTGGGGCA | Novel | NGG |
| CCTTGTTGGGATTGAAGTCC | Novel | NAA |
| CCTTTGGATAAAACCTAGCA | Novel | NGC |
| CCTTTTGGGGTGGAGCCCTC | Novel | NGG |
| CGAAGGTTTGGTACAGCAAC | Novel | NGG |
| CGAAGTGCACACGGTCCGGC | Novel | NGA |
| CGAATTTTGGCCAAGACACA | Novel | NGG |
| CGAATTTTGGCCAAGACACAC | Novel | NNNRRT |
| CGACCCCGAGAAGGGTCGTC | Novel | NGC |
| CGACCCTTATAAAGAATTTG | Novel | NAG |
| CGACCCTTCTCGGGGTCGCT | Novel | NGG |
| CGACCTTGAGGCATACTTCA | Novel | NAG |
| CGACGAGGCAGGTCCCCTAG | Novel | NAG |
| CGACGTGCAGAGGTGAAGCG | Novel | NAG |
| CGAGAAAGTGAAAGCCTGCT | Novel | NAG |
| CGAGAAGATTGACGATAAGG | Novel | NAG |
| CGAGCAAAACAAGCGGCTAG | Novel | NAG |
| CGAGGCAGGTCCCCTAGAAG | Novel | NAG |
| CGAGGGAGTTCTTCTTCTAG | Novel | NGG |
| CGATAACCAGGACAAGTTGG | M58 | NGG |
| CGATGTATATTTGCGGGAGA | Novel | NGA |
| CGATTGAGACCTTCGTCTGC | Novel | NAG |
| CGATTGGTGGAGGCAGGAGG | Novel | NGG |
| CGCAACCCCCACTGGCTGGGG | Novel | NNNRRT |
| CGCACGGAGGCCTTTTGGGG | Novel | NGG |
| CGCAGAAGATCTCAATCTCG | Novel | NGA |
| CGCAGACCAATTTATGCCTA | Novel | NAG |
| CGCAGAGTCTAGACTCGTGG | M35 | NGG |
| CGCAGGATAACCACATTGTG | Novel | NAA |
| CGCAGGATTCAGCGCCGACG | Novel | NGA |
| CGCAGGGTCCCCAATCCTCG | Novel | NGA |
| CGCAGGGTCCCCAATCCTCGA | Novel | NNNRRT |
| CGCAGTATGGATCGGCAGAG | Novel | NAG |
| CGCATGGAGACCACCGTGAA | Novel | NGC |
| CGCCAACAAGGTAGGAGCTG | Novel | NAG |
| CGCCCACCGAATGTTGCCCA | E95 | NGG |
| CGCCCCGTGGTCGGTCGGAA | Novel | NGG |
| CGCCGACGGGACGTAAACAA | Novel | NGG |
| CGCCGCGTCGCAGAAGATCT | Novel | NAA |
| CGCCTCAGCTCTGTATCGGG | Novel | NAG |
| CGCCTCCTGCCTCCACCAAT | Novel | NGC |
| CGCCTCGCAGACGAAGGTCT | Novel | NAA |
| CGCGCTGATGGCCCATGACC | Novel | NAG |
| CGCGTAAAGAGAGGTGCGCCC | Novel | NNNRRT |
| CGCGTGCGTGGAACCTTTTC | Novel | NGC |
| CGGAAAATTCCTATGGGAGT | Novel | NGG |
| CGGAAACTACTGTTGTTAGA | Novel | NGA |
| CGGAACGGCAGACGGAGAAG | Novel | NGG |
| CGGAAGTGTTGATAGGATAG | Novel | NGG |
| CGGACGACCCTTCTCGGGGT | Novel | NGC |
| CGGCAGACGGAGAAGGGGAC | Novel | NAG |
| CGGCGATTGAGACCTTCGTC | Novel | NGC |
| CGGCTAGGAGTTCCGCAGTA | Novel | NGG |
| CGGGAAGCCTTAGAGTCTCC | Novel | NGA |
| CGGGCAACGGGGTAAAGGTT | Novel | NAG |
| CGGGCTGAGGCCCACTCCCA | Novel | NAG |
| CGGGCTGAGGCCCACTCCCAT | Novel | NNGRRT |
| CGGGCTGAGGCCCACTCCCAT | Novel | NNNRRT |
| CGGGCTGGGTTTCACCCCAC | Novel | NGC |
| CGGGGAGTCCGCGTAAAGAG | Novel | NGG |
| CGGGGTTTTTCTTGTTGACA | Novel | NGA |
| CGGGTATATTATATAAGAGA | Novel | NAA |
| CGGTAAAAAGGGACTCAAGA | Novel | NGC |
| CGGTCGGAACGGCAGACGGA | Novel | NAA |
| CGGTGGGGTGAAACCCAGCC | Novel | NGA |
| CGGTTTCTCTTCCAAAAGTG | Novel | NGA |
| CGTAAACAAAGGACGTCCCG | Novel | NGC |
| CGTACTGAAGGAAAGAAGTC | Novel | NGA |
| CGTCAATCTTCTCGAGGATT | Novel | NGG |
| CGTCAGCAAACACTTGGCAC | Novel | NGA |
| CGTCCCGCGCAGGATCCAGT | Novel | NGG |
| CGTCCGAAGGTTTGGTACAG | Novel | NAA |
| CGTCGCATGGAGACCACCGT | Novel | NAA |
| CGTCTAACAACAGTAGTTTC | Novel | NGG |
| CGTCTAACAACAGTAGTTTCC | Novel | NNNRRT |
| CGTCTGGCCAGGTGTCCTTGT | Novel | NNGRRT |
| CGTCTGGCCAGGTGTCCTTGT | Novel | NNNRRT |
| CGTGAACGCCCACCGAATGT | Novel | NGC |
| CGTGCAGAGGTGAAGCGAAG | Novel | NGC |
| CGTGCGGTGGGGTGAAACCC | Novel | NGC |
| CGTGCTGGTGGTTGAGGATCC | Novel | NNGRRT |
| CGTGCTGGTGGTTGAGGATCC | Novel | NNNRRT |
| CGTGTGTCTTGGCCAAAATT | Novel | NGC |
| CGTTCACGGTGGTCTCCATG | Novel | NGA |
| CGTTCCGACCGACCACGGGG | Novel | NGC |
| CGTTGACATTGCAGAGAGTC | Novel | NAA |
| CGTTGACATTGCAGAGAGTCC | Novel | NNGRRT |
| CGTTGACATTGCAGAGAGTCC | Novel | NNNRRT |
| CGTTGCCGGGCAACGGGGTA | Novel | NAG |
| CTAACAACAGTAGTTTCCGG | Novel | NAG |
| CTAATATGGGCCTAAAGTTC | Novel | NGG |
| CTAATGACTCTAGCTACCTGG | Novel | NNGRRT |
| CTAATGACTCTAGCTACCTGG | Novel | NNNRRT |
| CTACCTTGTTGGCGTCTGGC | Novel | NAG |
| CTACGAACCACTGAACAAAT | Novel | NGC |
| CTACTAGGTCTCTAGATGCT | Novel | NGA |
| CTACTGTTGTTAGACGACGA | Novel | NGC |
| CTACTGTTGTTAGACGACGAG | Novel | NNNRRT |
| CTAGAAGATCTCGTACTGAA | Novel | NGA |
| CTAGACTCGTGGTGGACTTCT | E9 | NNNRRT |
| CTAGACTCTGCGGTATTGTG | Novel | NGG |
| CTAGAGTCATTAGTTCCCCC | Novel | NAG |
| CTAGCAGGCATAATCAATTG | Novel | NAA |
| CTAGCCGCTTGTTTTGCTCG | Novel | NAG |
| CTAGCTACCTGGGTGGGTGT | Novel | NAA |
| CTAGGTTTTATCCAAAGGTTA | Novel | NNNRRT |
| CTAGTAAACTGAGCCAGGAG | Novel | NAA |
| CTAGTAGTCAGTTATGTCAAC | Novel | NNNRRT |
| CTATAACGGTTTCTCTTCCA | Novel | NAA |
| CTATCCTATCAACACTTCCG | Novel | NAA |
| CTATGTGTTGTTTCTCTCTTA | Novel | NNNRRT |
| CTATTAACAGGCCTATTGAT | Novel | NGG |
| CTATTGATTGGAAAGTATGT | Novel | NAA |
| CTATTTACACACTCTATGGA | Novel | NGG |
| CTCAAGATGCTGTACAGACT | Novel | NGG |
| CTCAATCGCCGCGTCGCAGA | M77 | NGA |
| CTCAATCTCGGGAACCTCAAT | Novel | NNNRRT |
| CTCACAATACCGCAGAGTCT | Novel | NGA |
| CTCACCAACCTCCTGTCCTC | Novel | NAA |
| CTCACCATACTGCACTCAGG | Novel | NAA |
| CTCAGGCTCAGGGCATACTA | Novel | NAA |
| CTCATCCTCAGGCCATGCAG | Novel | NGG |
| CTCCAACTTGTCCTGGTTAT | Novel | NGC |
| CTCCAAGCTGTGCCTTGGGT | Novel | NGC |
| CTCCACCAATCGCCAGACAG | Novel | NAA |
| CTCCACCCCAAAAGGCCTCCG | Novel | NNNRRT |
| CTCCAGACCTGCTGCGAGCA | Novel | NAA |
| CTCCATGCGACGTGCAGAGG | M92 | NGA |
| CTCCATGTTCAGCGCAGGGTC | Novel | NNNRRT |
| CTCCCATAGGAATTTTCCGA | Novel | NAG |
| CTCCCTCGCCTCGCAGACGA | Novel | NGG |
| CTCCTACCTTGTTGGCGTCT | Novel | NGC |
| CTCCTGCCTCCACCAATCGC | Novel | NAG |
| CTCCTGGCTCAGTTTACTAG | Novel | NGC |
| CTCGAGAAGATTGACGATAA | Novel | NGG |
| CTCGAGGATTGGGGACCCTG | Novel | NGC |
| CTCTAAGGCTTCCCGATACA | Novel | NAG |
| CTCTAGATGCTGGATCTTCC | Novel | NAA |
| CTCTATAACGGTTTCTCTTC | Novel | NAA |
| CTCTATAACGGTTTCTCTTCC | Novel | NNNRRT |
| CTCTCACTCTGGGATCTTGC | Novel | NGA |
| CTCTCGTCCCCTTCTCCGTC | Novel | NGC |
| CTCTCTGCAATGTCAACGAC | Novel | NGA |
| CTCTGAAGGCTGGATCCAAC | Novel | NGG |
| CTCTGAAGGCTGGATCCAACT | Novel | NNNRRT |
| CTCTGCAAGATCCCAGAGTG | Novel | NGA |
| CTCTGCCGATCCATACTGCG | Novel | NAA |
| CTCTGGGATCTTGCAGAGTT | Novel | NGG |
| CTCTGGGATTCTTTCCCGACC | Novel | NNNRRT |
| CTCTGTATCGGGAAGCCTTA | Novel | NAG |
| CTCTTATGTAAGACCTTGGG | Novel | NAA |
| CTCTTCCAAAAGTGAGACAA | Novel | NAA |
| CTCTTTGTTTTGTTAGGGTT | Novel | NAA |
| CTGAAAGCCAAACAGTGGGG | Novel | NAA |
| CTGAACTGGAGCCACCAGCA | Novel | NGG |
| CTGAACTGGAGCCACCAGCAG | Novel | NNNRRT |
| CTGAAGGAAAGAAGTCAGAA | Novel | NGC |
| CTGACGCAACCCCCACTGGC | Novel | NGG |
| CTGACTACTAGGTCTCTAGA | Novel | NGC |
| CTGACTTCTTTCCTTCAGTA | Novel | NGA |
| CTGAGCCAGGAGAAACGGGC | M70 | NGA |
| CTGAGGATGAGTGTTTCTCA | Novel | NAG |
| CTGAGGGCTCCACCCCAAAA | Novel | NGC |
| CTGAGTGCAGTATGGTGAGG | Novel | NGA |
| CTGCAAGATCCCAGAGTGAG | Novel | NGG |
| CTGCATGACTACTGCTCAAG | Novel | NAA |
| CTGCCAACTGGATCCTGCGC | M191 | NGG |
| CTGCCGGACCGTGTGCACTT | Novel | NGC |
| CTGCCGTTCCGACCGACCAC | Novel | NGG |
| CTGCCTCCACCAATCGCCAG | Novel | NNAGGA |
| CTGCCTTCCTGTCTGGCGAT | Novel | NGG |
| CTGCGAATTTTGGCCAAGACA | Novel | NNNRRT |
| CTGCGAGCAAAACAAGCGGC | Novel | NAG |
| CTGCGAGCAAAACAAGCGGCT | Novel | NNGRRT |
| CTGCGAGCAAAACAAGCGGCT | Novel | NNNRRT |
| CTGCTGCGAGCAAAACAAGC | Novel | NGC |
| CTGCTGGTGGCTCCAGTTCA | Novel | NGA |
| CTGGAATTAGAGGACAAACG | Novel | NGC |
| CTGGATCCAACTGGTGGTCG | Novel | NGA |
| CTGGCCAGACGCCAACAAGG | Novel | NAG |
| CTGGCCAGGTGTCCTTGTTG | Novel | NGA |
| CTGGCCGTTGCCGGGCAACG | Novel | NGG |
| CTGGCGATTGGTGGAGGCAG | Novel | NAG |
| CTGGCTGGGGCTTGGTCATG | Novel | NGC |
| CTGGGAGGAGTTGGGGGAGG | Novel | NGA |
| CTGGGGGGAACTAATGACTC | Novel | NAG |
| CTGGGTGGGTGTTAATTTGG | Novel | NAG |
| CTGGGTTTCACCCCACCGCA | Novel | NGG |
| CTGGTGCGCAGACCAATTTA | Novel | NGC |
| CTGTAACACGAGAAGGGGTC | Novel | NNAGGA |
| CTGTACAGACTTGGCCCCCA | Novel | NNACCA |
| CTGTACCAAACCTTCGGACG | Novel | NAA |
| CTGTATTTCCCTGCTGGTGGC | Novel | NNNRRT |
| CTGTCTGGCGATTGGTGGAG | Novel | NNAGGA |
| CTGTCTTTAATCCTCATTGG | Novel | NAA |
| CTGTGCCAAGTGTTTGCTGA | Novel | NGC |
| CTGTGCCTTGGGTGGCTTTG | Novel | NGG |
| CTGTGCTGCCAACTGGATCC | Novel | NGC |
| CTGTGTTTGCTCTGAAGGCT | Novel | NGA |
| CTGTTAATAGGAAGTTTTCT | Novel | NAA |
| CTGTTCAAGCCTCCAAGCTG | Novel | NGC |
| CTGTTGCTGTACCAAACCTT | Novel | NGG |
| CTGTTGTTAGACGACGAGGC | Novel | NGG |
| CTGTTTGGCTTTCAGTTATA | Novel | NGG |
| CTGTTTGTTTAAAGACTGGG | Novel | NGG |
| CTTACAAGGCCTTTCTGTGT | Novel | NAA |
| CTTACAAGGCCTTTCTGTGTA | Novel | NNNRRT |
| CTTACAGTTAATGAGAAAAG | Novel | NAG |
| CTTATCCAATGGTAAATATT | Novel | NGG |
| CTTATCGTCAATCTTCTCGA | Novel | NGA |
| CTTATGAGTCCAAGGAATAC | Novel | NAA |
| CTTCAAAGACTGTTTGTTTA | Novel | NAG |
| CTTCACCTCTGCACGTCGCA | Novel | NGG |
| CTTCCAAATTAACACCCACC | Novel | NAG |
| CTTCCAATGACATAACCCAT | Novel | NAA |
| CTTCCGGAAACTACTGTTGT | Novel | NAG |
| CTTCCTATTAACAGGCCTAT | Novel | NGA |
| CTTCCTGTCTGGCGATTGGT | Novel | NGA |
| CTTCTCTCAATTTTCTAGGG | M87 | NGA |
| CTTCTGCGACGCGGCGATTG | Novel | NGA |
| CTTCTTTTCTCATTAACTGT | Novel | NAG |
| CTTGAACAGTAGGACATGAA | Novel | NAA |
| CTTGCCTGAGTGCAGTATGG | Novel | NGA |
| CTTGGCACAGACCTGGCCGT | Novel | NGC |
| CTTGGGTGGCTTTGGGGCAT | Novel | NGA |
| CTTGGTGTAAATGTATATTA | Novel | NGA |
| CTTGTAAGTTGGCGAGAAAG | Novel | NGA |
| CTTGTCAACAAGAAAAACCC | Novel | NGC |
| CTTGTCTCACTTTTGGAAGA | Novel | NAA |
| CTTGTTCATGTCCTACTGTT | Novel | NAA |
| CTTGTTGACAAGAATCCTCA | Novel | NAA |
| CTTTAAACAAACAGTCTTTG | Novel | NAG |
| CTTTAGGCCCATATTAGTGT | Novel | NGA |
| CTTTCCACCAGCAATCCTCT | Novel | NGG |
| CTTTCGGAAAATTCCTATGG | Novel | NAG |
| CTTTCTGTGTAAACAATACC | Novel | NGA |
| CTTTGAGAAACACTCATCCT | Novel | NAG |
| CTTTGGATAAAACCTAGCAGG | Novel | NNNRRT |
| CTTTGTACTAGGAGGCTGTA | Novel | NGC |
| CTTTGTTTACGTCCCGTCGG | Novel | NGC |
| CTTTTCTCATTAACTGTAAG | Novel | NGG |
| CTTTTGGAAGAGAAACCGTTA | Novel | NNGRRT |
| CTTTTGGAAGAGAAACCGTTA | Novel | NNNRRT |
| CTTTTGGGGTGGAGCCCTCA | Novel | NGC |
| GAAAAACCCCGCCTGTAACA | Novel | NGA |
| GAAAAGATGGTGTTTTCCAA | Novel | NGA |
| GAAAAGATGGTGTTTTCCAA | Novel | NNAGGA |
| GAAAAGATGGTGTTTTCCAAT | Novel | NNGRRT |
| GAAAAGATGGTGTTTTCCAAT | Novel | NNNRRT |
| GAAAATTGAGAGAAGTCCACC | Novel | NNGRRT |
| GAAAATTGAGAGAAGTCCACC | Novel | NNNRRT |
| GAAAATTGGTAACAGCGGTA | Novel | NAA |
| GAAACACTCATCCTCAGGCCA | Novel | NNNRRT |
| GAAACCCAGCCCGAATGCTC | Novel | NAG |
| GAAAGACACCAAATACTCTA | Novel | NAA |
| GAAAGACACCAAATACTCTAT | Novel | NNNRRT |
| GAAAGCCAAACAGTGGGGGA | Novel | NAG |
| GAAAGCCCAGGATGATGGGA | M37 | NGG |
| GAAAGCCCAGGATGATGGGAT | M4 | NNGRRT |
| GAAAGCCCAGGATGATGGGAT | Novel | NNNRRT |
| GAAAGCCCTACGAACCACTGA | Novel | NNNRRT |
| GAAAGGCCTTGTAAGTTGGC | Novel | NAG |
| GAAAGTATGTCAACGAATTG | Novel | NGG |
| GAAAGTGAAAGCCTGCTTAGA | Novel | NNGRRT |
| GAAAGTGAAAGCCTGCTTAGA | Novel | NNNRRT |
| GAAATACAGGCCTCTCACTC | Novel | NGG |
| GAACAAGAGATGATTAGGCA | Novel | NAG |
| GAACAAGATCTACAGCATGG | Novel | NGC |
| GAACAATGCTCAGGAGACTC | Novel | NAA |
| GAACACATCATACAAAAAAT | Novel | NAA |
| GAACAGGGTTTACTGCTCCT | Novel | NAA |
| GAACAGTAGGACATGAACAA | Novel | NAG |
| GAACATCACATCAGGATTCC | Novel | NAG |
| GAACATGGAGAACATCACAT | Novel | NAG |
| GAACCTGCATGACTACTGCT | Novel | NAA |
| GAACCTGCATGACTACTGCT | Novel | NNAGGA |
| GAACCTTTACCCCGTTGCCC | Novel | NGC |
| GAACGCCCACCGAATGTTGCC | Novel | NNNRRT |
| GAACTACCGTGTGTCTTGGC | Novel | NAA |
| GAACTCCCTCGCCTCGCAGA | Novel | NGA |
| GAACTCCCTCGCCTCGCAGAC | E10 | NNNRRT |
| GAACTCCTAGCCGCTTGTTT | Novel | NGC |
| GAACTGGAGCCACCAGCAGG | Novel | NAA |
| GAAGAACCAACAAGAAGATG | Novel | NGG |
| GAAGAACTCCCTCGCCTCGC | E11 | NGA |
| GAAGAAGAACTCCCTCGCCT | Novel | NGC |
| GAAGATCTCAATCTCGGGAAC | M14 | NNNRRT |
| GAAGATCTCGTACTGAAGGA | Novel | NAG |
| GAAGATCTCGTACTGAAGGAA | Novel | NNNRRT |
| GAAGATGAGGCATAGCAGCA | Novel | NGA |
| GAAGATTGACGATAAGGGAG | Novel | NGG |
| GAAGATTGACGATAAGGGAGA | Novel | NNNRRT |
| GAAGCGAAGTGCACACGGTC | Novel | NGG |
| GAAGGAAAGAAGTCAGAAGG | Novel | NAA |
| GAAGGCACAGACGGGGAGTC | Novel | NGC |
| GAAGGCGGGTATATTATATA | Novel | NGA |
| GAAGGGGACGAGAGAGTCCC | Novel | NAG |
| GAAGGGTCGTCCGCAGGATT | Novel | NAG |
| GAAGGTCTCAATCGCCGCGT | Novel | NGC |
| GAAGGTTTGGTACAGCAACA | Novel | NGA |
| GAAGTCAGAAGGCAAAAACG | Novel | NGA |
| GAAGTTATGGGTCCTTGCCA | Novel | NAA |
| GAATACTAACATTGAGGTTCC | Novel | NNNRRT |
| GAATATGGTGACCCACAAAA | Novel | NGA |
| GAATCCACACTCCGAAAGACA | M12 | NNNRRT |
| GAATCCCAGAGGATTGCTGG | Novel | NGG |
| GAATTGCTTGCCTGAGTGCAG | Novel | NNNRRT |
| GACAAAAGAAAATTGGTAAC | Novel | NGC |
| GACAAACGGGCAACATACCT | Novel | NGA |
| GACAAACGGGCAACATACCTT | Novel | NNNRRT |
| GACAACAGAGTTATCAGTCC | Novel | NGA |
| GACAACAGAGTTATCAGTCCC | Novel | NNNRRT |
| GACAAGAAATGTGAAACCAC | Novel | NAG |
| GACAAGAATCCTCACAATAC | Novel | NGC |
| GACACACGGTAGTTCCCCCT | Novel | NGA |
| GACACACGGTAGTTCCCCCTA | Novel | NNNRRT |
| GACACATCCAGCGATAACCA | M67 | NGA |
| GACACCTGGCCAGACGCCAA | Novel | NAA |
| GACACTATTTACACACTCTA | Novel | NGG |
| GACAGGAAGGCAGCCTACCC | Novel | NGC |
| GACAGGTACAGTAGAAGAAT | Novel | NAA |
| GACATAACCCATAAAATTCA | Novel | NAG |
| GACATACTTTCCAATCAATA | Novel | NGC |
| GACATGAACAAGAGATGATT | Novel | NGG |
| GACCAAGCCCCAGCCAGTGG | Novel | NGG |
| GACCACCGTGAACGCCCACC | Novel | NAA |
| GACCCCGAGAAGGGTCGTCC | Novel | NNAGGA |
| GACCCCGAGAAGGGTCGTCCG | Novel | NNGRRT |
| GACCCCGAGAAGGGTCGTCCG | Novel | NNNRRT |
| GACCCCTTCTCGTGTTACAGG | Novel | NNGRRT |
| GACCCCTTCTCGTGTTACAGG | Novel | NNNRRT |
| GACCCTGCGCTGAACATGGA | Novel | NAA |
| GACCCTTATAAAGAATTTGG | Novel | NGC |
| GACCCTTCTCGGGGTCGCTT | Novel | NGG |
| GACCTAGTAGTCAGTTATGT | Novel | NAA |
| GACCTGCTGCGAGCAAAACA | Novel | NGC |
| GACCTGGCCGTTGCCGGGCAA | Novel | NNGRRT |
| GACCTGGCCGTTGCCGGGCAA | Novel | NNNRRT |
| GACCTTCGTCTGCGAGGCGA | Novel | NGG |
| GACCTTGAGGCATACTTCAA | Novel | NGA |
| GACCTTGGGCAACATTCGGT | Novel | NGG |
| GACGACGAGGCAGGTCCCCT | M74 | NGA |
| GACGAGGCAGGTCCCCTAGA | M75 | NGA |
| GACGCAACCCCCACTGGCTG | Novel | NGG |
| GACGCCAACAAGGTAGGAGC | M53 | NGG |
| GACGGGGAGTCCGCGTAAAG | Novel | NGA |
| GACGTAAACAAAGGACGTCC | Novel | NGC |
| GACTACTGCCTCTCCCTTATC | Novel | NNNRRT |
| GACTCGTGGTGGACTTCTCT | Novel | NAA |
| GACTCTAAGGCTTCCCGATA | Novel | NAG |
| GACTGGGAGGAGTTGGGGGA | Novel | NGA |
| GACTGTTTGTTTAAAGACTG | Novel | NGA |
| GACTGTTTGTTTAAAGACTG | Novel | NNAGGA |
| GACTTCTCTCAATTTTCTAG | E12 | NGG |
| GACTTCTTTCCTTCAGTACG | Novel | NGA |
| GAGAAAGTGAAAGCCTGCTT | Novel | NGA |
| GAGAAGATTGACGATAAGGG | Novel | NGA |
| GAGAAGTCCACCACGAGTCT | M66 | NGA |
| GAGACCTTCGTCTGCGAGGC | Novel | NAG |
| GAGAGAGTCCCAAGCGACCC | Novel | NGA |
| GAGAGGCAGTAGTCGGAACA | Novel | NGG |
| GAGAGTAACTCCACAGTAGCT | Novel | NNNRRT |
| GAGAGTCCCAAGCGACCCCG | Novel | NGA |
| GAGATGATTAGGCAGAGGTG | Novel | NAA |
| GAGATGATTAGGCAGAGGTGA | Novel | NNNRRT |
| GAGATTGAGATCTTCTGCGA | Novel | NGC |
| GAGCAGTAGTCATGCAGGTT | Novel | NGG |
| GAGCCACCAGCAGGGAAATA | Novel | NAG |
| GAGCCAGGAGAAACGGGCTG | Novel | NGG |
| GAGCCTGAGGGCTCCACCCC | Novel | NAA |
| GAGCTGAGGCGGTATCTAGA | Novel | NGA |
| GAGGACAAACGGGCAACATAC | Novel | NNNRRT |
| GAGGACAACAGAGTTATCAGT | Novel | NNNRRT |
| GAGGACAGGAGGTTGGTGAG | Novel | NGA |
| GAGGACTCTTGGACTCTCTG | Novel | NAA |
| GAGGAGCCGAAAAGGTTCCA | Novel | NGC |
| GAGGAGTTGGGGGAGGAGAT | Novel | NAG |
| GAGGATTCTTGTCAACAAGA | Novel | NAA |
| GAGGATTGGGGACCCTGCGC | Novel | NGA |
| GAGGCAGGAGGCGGATTTGC | Novel | NGG |
| GAGGCAGGTCCCCTAGAAGA | Novel | NGA |
| GAGGCATAGCAGCAGGATGA | Novel | NGA |
| GAGGCATAGCAGCAGGATGA | Novel | NNAGGA |
| GAGGCTTGAACAGTAGGACA | Novel | NGA |
| GAGGGAGTTCTTCTTCTAGG | Novel | NGA |
| GAGGTGAAAAAGTTGCATGG | Novel | NGC |
| GAGGTGAAAAAGTTGCATGGT | Novel | NNNRRT |
| GAGGTGAAGCGAAGTGCACA | Novel | NGG |
| GAGTAACTCCACAGTAGCTC | Novel | NAA |
| GAGTCATTAGTTCCCCCCAG | Novel | NAA |
| GAGTCCAAGGAATACTAACAT | Novel | NNNRRT |
| GAGTCCCAAGCGACCCCGAG | Novel | NAG |
| GAGTCCCTTTTTACCGCTGTT | Novel | NNNRRT |
| GAGTGCGAATCCACACTCCG | Novel | NAA |
| GAGTTTGGTGAAAGGTTGTG | Novel | NAA |
| GATAACCACATTGTGTAAAT | Novel | NGG |
| GATAACCAGGACAAGTTGGA | M89 | NGA |
| GATAAGGGAGAGGCAGTAGT | Novel | NGG |
| GATAATGTTTGCTCCAGACC | Novel | NGC |
| GATACGATGTATATTTGCGG | Novel | NAG |
| GATAGGATAGGGGCATTTGG | Novel | NGG |
| GATAGTCCAGAAGAACCAAC | Novel | NAG |
| GATCCAACTGGTGGTCGGGA | Novel | NAG |
| GATCCATACTGCGGAACTCC | Novel | NAG |
| GATCCTCAACCACCAGCACG | Novel | NGA |
| GATCCTCAACCACCAGCACG | Novel | NNACCA |
| GATCGGCAGAGGAGCCGAAA | Novel | NGG |
| GATCTCAATCTCGGGAACCT | Novel | NAA |
| GATCTCGTACTGAAGGAAAG | Novel | NAG |
| GATCTTCTAGATACCGCCTC | Novel | NGC |
| GATCTTGCAGAGTTTGGTGA | Novel | NAG |
| GATGAGAAGGCACAGACGGG | Novel | NAG |
| GATGAGGCATAGCAGCAGGA | Novel | NGA |
| GATGAGTGTTTCTCAAAGGT | Novel | NGA |
| GATGATGTGGTATTGGGGGC | Novel | NAA |
| GATGATTAGGCAGAGGTGAA | Novel | NAA |
| GATGGCCCATGACCAAGCCC | Novel | NAG |
| GATGTGATGTTCTCCATGTT | Novel | NAG |
| GATGTTCTCCATGTTCAGCG | Novel | NAG |
| GATTAAAGACAGGTACAGTA | Novel | NAA |
| GATTAAAGACAGGTACAGTAG | Novel | NNGRRT |
| GATTAAAGACAGGTACAGTAG | Novel | NNNRRT |
| GATTAAAGGTCTTTGTACTA | Novel | NGA |
| GATTAACTAGATGTTCTGGA | Novel | NAA |
| GATTCAGCGCCGACGGGACG | Novel | NAA |
| GATTGAATACATGCATACAA | Novel | NGG |
| GATTGACGATAAGGGAGAGG | Novel | NAG |
| GATTGACGATAAGGGAGAGGC | Novel | NNNRRT |
| GATTGAGACCTTCGTCTGCG | Novel | NGG |
| GATTGAGATCTTCTGCGACG | Novel | NGG |
| GATTGAGATCTTCTGCGACGC | Novel | NNNRRT |
| GATTGCAATTGATTATGCCTG | M18 | NNNRRT |
| GATTGCTGGTGGAAAGATTC | Novel | NGC |
| GATTGGAGGTTGGGGACTGC | Novel | NAA |
| GATTGGGACTTCAATCCCAA | Novel | NAA |
| GATTGGGACTTCAATCCCAA | Novel | NNAGGA |
| GATTGGTGGAGGCAGGAGGC | Novel | NGA |
| GATTTGCTGTGTTTGCTCTG | Novel | NAG |
| GCAAACACTTGGCACAGACC | Novel | NGG |
| GCAAACATTATCGGGACTGA | Novel | NAA |
| GCAAAGAATTGCTTGCCTGAG | Novel | NNNRRT |
| GCAAAGTTTGTAGTATGCCC | Novel | NGA |
| GCAAATATACATCGTATCCA | Novel | NGG |
| GCAAATCCAGATTGGGACTT | Novel | NAA |
| GCAAATCCGCCTCCTGCCTCC | Novel | NNNRRT |
| GCAACAGGAGGGATACATAG | Novel | NGG |
| GCAACATACCTTGATAGTCC | Novel | NGA |
| GCAACGGCCAGGTCTGTGCC | Novel | NAG |
| GCAACTTTTTCACCTCTGCC | Novel | NAA |
| GCAAGCAATTCTTTGCTGGG | M45 | NGG |
| GCAATGTCAACGACCGACCT | Novel | NGA |
| GCAATTTCCGTCCGAAGGTT | Novel | NGG |
| GCACACGGTCCGGCAGATGA | Novel | NAA |
| GCACAGACCTGGCCGTTGCC | Novel | NGG |
| GCACAGACGGGGAGTCCGCG | Novel | NAA |
| GCACAGCCTAGCAGCCATGGA | Novel | NNNRRT |
| GCACAGCTTGGAGGCTTGAA | Novel | NAG |
| GCACGGAGGCCTTTTGGGGT | Novel | NGA |
| GCACGGGACCATGCCGAACC | Novel | NGC |
| GCACTAGTAAACTGAGCCAG | Novel | NAG |
| GCACTCCTCCAGCTTATAGA | Novel | NNACCA |
| GCAGAAGATCTCAATCTCGG | Novel | NAA |
| GCAGACACATCCAGCGATAA | Novel | NNAGGA |
| GCAGACCAATTTATGCCTAC | Novel | NGC |
| GCAGACGGAGAAGGGGACGA | Novel | NAG |
| GCAGAGGTGAAAAAGTTGCA | Novel | NGG |
| GCAGAGGTGAAGCGAAGTGCA | Novel | NNNRRT |
| GCAGAGTCTAGACTCGTGGT | M65 | NGA |
| GCAGATGAGAAGGCACAGAC | Novel | NGG |
| GCAGATGAGAAGGCACAGACG | Novel | NNGRRT |
| GCAGATGAGAAGGCACAGACG | Novel | NNNRRT |
| GCAGCACAGCCTAGCAGCCA | Novel | NGG |
| GCAGGAGGCGGATTTGCTGG | Novel | NAA |
| GCAGGATAACCACATTGTGT | Novel | NAA |
| GCAGGATCCAGTTGGCAGCA | Novel | NAG |
| GCAGGCTTTCACTTTCTCGC | Novel | NAA |
| GCAGGGTCCCCAATCCTCGA | Novel | NAA |
| GCAGGTTCGGCATGGTCCCG | Novel | NGC |
| GCAGGTTCGGCATGGTCCCGT | Novel | NNNRRT |
| GCAGTAGTCATGCAGGTTCGG | Novel | NNNRRT |
| GCAGTATGGATCGGCAGAGG | Novel | NGC |
| GCATAAATTGGTCTGCGCAC | Novel | NAG |
| GCATACAAGGGCATTAACGC | Novel | NGG |
| GCATAGCAGCAGGATGAAGA | Novel | NGA |
| GCATAGCAGCAGGATGAAGAG | Novel | NNNRRT |
| GCATCTAGAGACCTAGTAGT | Novel | NAG |
| GCATGGACATCGACCCTTAT | Novel | NAA |
| GCATGGACATCGACCCTTATA | Novel | NNGRRT |
| GCATGGACATCGACCCTTATA | Novel | NNNRRT |
| GCATGTATTCAATCTAAGCA | Novel | NGC |
| GCATTTGGTGGTCTATAAGC | Novel | NGG |
| GCCAACAAGGTAGGAGCTGG | Novel | NGC |
| GCCAAGTCTGTACAGCATCT | Novel | NGA |
| GCCACAAGAACACATCATAC | Novel | NAA |
| GCCACAAGAACACATCATACA | M28 | NNNRRT |
| GCCACCAGCAGGGAAATACA | Novel | NGC |
| GCCACCCAAGGCACAGCTTG | Novel | NAG |
| GCCAGACGCCAACAAGGTAG | Novel | NAG |
| GCCAGGTGTCCTTGTTGGGAT | Novel | NNNRRT |
| GCCAGTGGGGGTTGCGTCAG | Novel | NAA |
| GCCATTTGTTCAGTGGTTCG | Novel | NAG |
| GCCCAAGGTCTTACATAAGA | Novel | NGA |
| GCCCACTTACAGTTAATGAG | Novel | NAA |
| GCCCATGACCAAGCCCCAGC | Novel | NAG |
| GCCCCGTGGTCGGTCGGAAC | Novel | NGC |
| GCCCGTTTGTCCTCTAATTC | Novel | NAG |
| GCCGACGGGACGTAAACAAA | Novel | NGA |
| GCCGCAGACACATCCAGCGA | Novel | NAA |
| GCCGCAGACACATCCAGCGA | Novel | NNACCA |
| GCCGCTTGTTTTGCTCGCAG | Novel | NAG |
| GCCGGGCAACGGGGTAAAGGT | Novel | NNNRRT |
| GCCGTTCCGACCGACCACGG | Novel | NGC |
| GCCGTTGCCGGGCAACGGGG | Novel | NAA |
| GCCGTTGCCGGGCAACGGGGT | Novel | NNNRRT |
| GCCTAAAGTTCAGGCAACTCT | Novel | NNNRRT |
| GCCTACAAACTGTTCACATTT | Novel | NNNRRT |
| GCCTACAGCCTCCTAGTACA | Novel | NAG |
| GCCTCAGCCCGTTTCTCCTGG | Novel | NNNRRT |
| GCCTCAGCTCTGTATCGGGA | Novel | NGC |
| GCCTCCACCAATCGCCAGAC | Novel | NGG |
| GCCTCGTCGTCTAACAACAG | Novel | NAG |
| GCCTCTCACTCTGGGATCTTG | Novel | NNGRRT |
| GCCTCTCACTCTGGGATCTTG | Novel | NNNRRT |
| GCCTGAGGATGAGTGTTTCT | Novel | NAA |
| GCCTGAGGATGAGTGTTTCTC | Novel | NNNRRT |
| GCCTGAGGGCTCCACCCCAA | Novel | NAG |
| GCCTGCTAGGTTTTATCCAA | Novel | NGG |
| GCCTGCTTAGATTGAATACA | Novel | NGC |
| GCCTGTATTTCCCTGCTGGT | Novel | NGC |
| GCCTTCAGAGCAAACACAGC | Novel | NAA |
| GCCTTCTGACTTCTTTCCTT | Novel | NAG |
| GCCTTTTGGGGTGGAGCCCT | Novel | NAG |
| GCGAAGTGCACACGGTCCGG | Novel | NAG |
| GCGAATCCACACTCCGAAAG | Novel | NNACCA |
| GCGACGTGCAGAGGTGAAGC | Novel | NAA |
| GCGAGCAAAACAAGCGGCTA | Novel | NGA |
| GCGAGGGAGTTCTTCTTCTA | Novel | NGG |
| GCGATAACCAGGACAAGTTG | Novel | NAG |
| GCGATTGAGACCTTCGTCTG | Novel | NGA |
| GCGCAGGGTCCCCAATCCTC | Novel | NAG |
| GCGCCGACGGGACGTAAACA | Novel | NAG |
| GCGCGTGCGTGGAACCTTTT | Novel | NGG |
| GCGCTGATGGCCCATGACCA | Novel | NGC |
| GCGGGAGAGGACAACAGAGTT | Novel | NNNRRT |
| GCGGGGTTTTTCTTGTTGAC | Novel | NAG |
| GCGGGTATATTATATAAGAG | Novel | NGA |
| GCGTCAGCAAACACTTGGCA | Novel | NAG |
| GCTAGGCTGTGCTGCCAACT | Novel | NGA |
| GCTATGCCTCATCTTCTTGT | Novel | NGG |
| GCTCAGGAGACTCTAAGGCTT | Novel | NNNRRT |
| GCTCCAGACCTGCTGCGAGC | Novel | NAA |
| GCTCCAGCTCCTACCTTGTT | Novel | NGC |
| GCTCCTACCTTGTTGGCGTC | Novel | NGG |
| GCTCCTCTGCCGATCCATAC | Novel | NGC |
| GCTCCTGAACTGGAGCCACC | Novel | NGC |
| GCTCGCAGCAGGTCTGGAGC | Novel | NAA |
| GCTCTGTATCGGGAAGCCTT | Novel | NGA |
| GCTGAGGCCCACTCCCATAG | Novel | NAA |
| GCTGCCAACTGGATCCTGCG | M190 | NGG |
| GCTGCCCCATTTACACAATG | Novel | NGG |
| GCTGCGAGCAAAACAAGCGG | Novel | NNAGGA |
| GCTGCTAGGCTGTGCTGCCAA | Novel | NNGRRT |
| GCTGCTAGGCTGTGCTGCCAA | Novel | NNNRRT |
| GCTGCTATGCCTCATCTTCTT | E14 | NNNRRT |
| GCTGGATCCAACTGGTGGTC | Novel | NGG |
| GCTGGTGGCTCCAGTTCAGG | Novel | NGC |
| GCTGGTGGTTGAGGATCCTG | Novel | NAA |
| GCTGTACCAAACCTTCGGAC | M91 | NGA |
| GCTGTAGATCTTGTTCCCAA | Novel | NAA |
| GCTGTAGGCATAAATTGGTC | Novel | NGC |
| GCTGTGCCTTGGGTGGCTTT | Novel | NGG |
| GCTGTGTTTGCTCTGAAGGC | Novel | NGG |
| GCTTCCCGATACAGAGCTGA | Novel | NGC |
| GCTTGCCTGAGTGCAGTATGG | Novel | NNNRRT |
| GCTTGGAGGCTTGAACAGTA | Novel | NGA |
| GCTTGGTCATGGGCCATCAG | Novel | NGC |
| GCTTTCCCCCACTGTTTGGCT | Novel | NNNRRT |
| GCTTTCGGAAAATTCCTATG | Novel | NGA |
| GGAAAATTCCTATGGGAGTG | Novel | NGC |
| GGAAAGAAGTCAGAAGGCAA | Novel | NAA |
| GGAAAGATTCTGCCCCATGCT | Novel | NNNRRT |
| GGAAAGCCCTACGAACCACT | Novel | NAA |
| GGAAATACAGGCCTCTCACTC | Novel | NNGRRT |
| GGAAATACAGGCCTCTCACTC | Novel | NNNRRT |
| GGAACAAGATCTACAGCATG | M51 | NGG |
| GGAACAGGGTTTACTGCTCC | Novel | NGA |
| GGAACGGCAGACGGAGAAGG | Novel | NGA |
| GGAACTAATGACTCTAGCTAC | Novel | NNGRRT |
| GGAACTAATGACTCTAGCTAC | Novel | NNNRRT |
| GGAACTACCGTGTGTCTTGGC | Novel | NNNRRT |
| GGAAGATCCAGCATCTAGAGA | Novel | NNNRRT |
| GGAAGATGATAAAACGCCGC | Novel | NGA |
| GGAAGCCTTAGAGTCTCCTG | Novel | NGC |
| GGAAGGCGGGTATATTATAT | Novel | NAG |
| GGAAGTGTTGATAGGATAGG | Novel | NGC |
| GGAATTAGAGGACAAACGGG | Novel | NAA |
| GGACAAGTTGGAGGACAGGAG | Novel | NNNRRT |
| GGACACCTGGCCAGACGCCAA | Novel | NNNRRT |
| GGACATGAACAAGAGATGAT | Novel | NAG |
| GGACCCCTTCTCGTGTTACA | Novel | NGC |
| GGACCCTGCGCTGAACATGG | Novel | NGA |
| GGACCTGCCTCGTCGTCTAA | Novel | NAA |
| GGACCTGCCTCGTCGTCTAAC | Novel | NNNRRT |
| GGACTTCTCTCAATTTTCTA | E15 | NGG |
| GGAGACCACCGTGAACGCCCA | Novel | NNGRRT |
| GGAGACCACCGTGAACGCCCA | Novel | NNNRRT |
| GGAGAGGACAACAGAGTTAT | Novel | NAG |
| GGAGAGGCAGTAGTCGGAAC | Novel | NGG |
| GGAGCAAACATTATCGGGAC | Novel | NGA |
| GGAGCTGGAGCATTCGGGCT | Novel | NGG |
| GGAGGTTGGTGAGTGATTGG | Novel | NGG |
| GGAGTCCGCGTAAAGAGAGG | Novel | NGC |
| GGAGTGCGAATCCACACTCC | Novel | NAA |
| GGAGTTCCGCAGTATGGATC | Novel | NGC |
| GGATAAAACCTAGCAGGCATA | Novel | NNNRRT |
| GGATAACCACATTGTGTAAA | Novel | NGG |
| GGATACATAGAGGTTCCTTG | Novel | NGC |
| GGATACGATGTATATTTGCG | Novel | NGA |
| GGATCCAACTGGTGGTCGGG | Novel | NAA |
| GGATCCAACTGGTGGTCGGGA | Novel | NNGRRT |
| GGATCCAACTGGTGGTCGGGA | Novel | NNNRRT |
| GGATCCTCAACCACCAGCAC | Novel | NGG |
| GGATCCTGGAATTAGAGGAC | Novel | NAA |
| GGATCGGCAGAGGAGCCGAA | Novel | NAG |
| GGATCTTGCAGAGTTTGGTG | Novel | NAA |
| GGATGAAGAGGAAGATGATA | Novel | NAA |
| GGATGAGTGTTTCTCAAAGG | Novel | NGG |
| GGATGATGGGATGGGAATAC | Novel | NGG |
| GGATTAAAGACAGGTACAGT | Novel | NGA |
| GGATTCTTTCCCGACCACCAG | Novel | NNGRRT |
| GGATTCTTTCCCGACCACCAG | Novel | NNNRRT |
| GGATTTGCTGGCAAAGTTTG | Novel | NAG |
| GGATTTGCTGTGTTTGCTCT | Novel | NAA |
| GGCAACATACCTTGATAGTC | Novel | NAG |
| GGCAACGGCCAGGTCTGTGC | Novel | NAA |
| GGCAAGCAATTCTTTGCTGG | M44 | NGG |
| GGCACAGACCTGGCCGTTGC | Novel | NGG |
| GGCACTAGTAAACTGAGCCA | Novel | NGA |
| GGCAGACGGAGAAGGGGACG | Novel | NGA |
| GGCAGACGGAGAAGGGGACGA | Novel | NNGRRT |
| GGCAGACGGAGAAGGGGACGA | Novel | NNNRRT |
| GGCAGATGAGAAGGCACAGA | Novel | NGG |
| GGCAGCAAAACCCAAAAGACC | Novel | NNNRRT |
| GGCAGGAGGCGGATTTGCTGG | Novel | NNNRRT |
| GGCATACTACAAACTTTGCC | Novel | NGC |
| GGCATACTACAAACTTTGCCA | Novel | NNNRRT |
| GGCATAGCAGCAGGATGAAG | Novel | NGG |
| GGCATGGACATCGACCCTTA | Novel | NAA |
| GGCCAGACGCCAACAAGGTA | Novel | NGA |
| GGCCCACTTACAGTTAATGA | Novel | NAA |
| GGCCCATATTAGTGTTGACA | Novel | NAA |
| GGCCTCAGCCCGTTTCTCCT | Novel | NGC |
| GGCCTCCGTGCGGTGGGGTG | Novel | NAA |
| GGCCTCTCACTCTGGGATCT | Novel | NGC |
| GGCCTGTATTTCCCTGCTGG | Novel | NGG |
| GGCCTTGTAAGTTGGCGAGA | Novel | NAG |
| GGCGAGAAAGTGAAAGCCTGC | Novel | NNNRRT |
| GGCGAGGGAGTTCTTCTTCT | Novel | NGG |
| GGCGATTGGTGGAGGCAGGA | Novel | NGC |
| GGCGATTGGTGGAGGCAGGAG | Novel | NNGRRT |
| GGCGATTGGTGGAGGCAGGAG | Novel | NNNRRT |
| GGCGGATTTGCTGGCAAAGTT | Novel | NNNRRT |
| GGCGGGGTTTTTCTTGTTGA | Novel | NAA |
| GGCGGGGTTTTTCTTGTTGAC | Novel | NNGRRT |
| GGCGGGGTTTTTCTTGTTGAC | Novel | NNNRRT |
| GGCGGGTATATTATATAAGA | Novel | NAG |
| GGCGTTCACGGTGGTCTCCA | Novel | NGC |
| GGCTAGGAGTTCCGCAGTAT | Novel | NGA |
| GGCTCCAGTTCAGGAGCAGT | Novel | NAA |
| GGCTGAGGCCCACTCCCATA | Novel | NGA |
| GGCTGGATCCAACTGGTGGT | Novel | NGG |
| GGCTTCCCGATACAGAGCTG | Novel | NGG |
| GGCTTCCCGATACAGAGCTGA | Novel | NNNRRT |
| GGCTTTCAGTTATATGGATGA | Novel | NNNRRT |
| GGCTTTCGGAAAATTCCTAT | Novel | NGG |
| GGGAAAGAATCCCAGAGGAT | Novel | NGC |
| GGGAAAGAATCCCAGAGGATT | Novel | NNNRRT |
| GGGAAAGCCCTACGAACCAC | Novel | NGA |
| GGGAACAAGATCTACAGCAT | M50 | NGG |
| GGGAAGCCTTAGAGTCTCCT | Novel | NAG |
| GGGACCATGCCGAACCTGCA | Novel | NGA |
| GGGACCCTGCGCTGAACATG | Novel | NAG |
| GGGACTGCGAATTTTGGCCA | Novel | NGA |
| GGGAGAGGCAGTAGTCGGAA | Novel | NAG |
| GGGAGGAGTTGGGGGAGGAGA | Novel | NNNRRT |
| GGGATACATAGAGGTTCCTT | Novel | NAG |
| GGGATACATAGAGGTTCCTTG | Novel | NNNRRT |
| GGGATCTTGCAGAGTTTGGT | Novel | NAA |
| GGGATCTTGCAGAGTTTGGTG | Novel | NNNRRT |
| GGGCAACATTCGGTGGGCGTT | Novel | NNNRRT |
| GGGCAACGGGGTAAAGGTTC | Novel | NGG |
| GGGCAGAATCTTTCCACCAG | Novel | NAA |
| GGGCATACTACAAACTTTGC | Novel | NAG |
| GGGCCAAGTCTGTACAGCATC | Novel | NNGRRT |
| GGGCCAAGTCTGTACAGCATC | Novel | NNNRRT |
| GGGCCATCAGCGCGTGCGTG | Novel | NAA |
| GGGCCTCAGCCCGTTTCTCC | Novel | NGG |
| GGGCGCACCTCTCTTTACGC | Novel | NGA |
| GGGCTGAGGCCCACTCCCAT | Novel | NGG |
| GGGCTTGGTCATGGGCCATC | Novel | NGC |
| GGGCTTTCGGAAAATTCCTA | Novel | NGG |
| GGGCTTTCGGAAAATTCCTAT | Novel | NNGRRT |
| GGGCTTTCGGAAAATTCCTAT | Novel | NNNRRT |
| GGGGAACTACCGTGTGTCTT | Novel | NGC |
| GGGGACCCTGCGCTGAACAT | Novel | NGA |
| GGGGACGAGAGAGTCCCAAG | Novel | NGA |
| GGGGACTGCGAATTTTGGCC | Novel | NAG |
| GGGGCATTTGGTGGTCTATA | Novel | NGC |
| GGGGCGCACCTCTCTTTACG | Novel | NGG |
| GGGGCTTGGTCATGGGCCAT | Novel | NAG |
| GGGGGAACTACCGTGTGTCT | Novel | NGG |
| GGGGGAGGAGATTAGATTAA | Novel | NGG |
| GGGGTGAAACCCAGCCCGAA | Novel | NGC |
| GGGGTGGAGCCCTCAGGCTC | Novel | NGG |
| GGGGTTTTTCTTGTTGACAA | Novel | NAA |
| GGGTAGGCTGCCTTCCTGTC | Novel | NGG |
| GGGTAGGCTGCCTTCCTGTCT | Novel | NNNRRT |
| GGGTATATTATATAAGAGAG | Novel | NAA |
| GGGTATTAAACCTTATTATC | Novel | NAG |
| GGGTCACCATATTCTTGGGAA | Novel | NNNRRT |
| GGGTCCTAGGAATCCTGATG | Novel | NGA |
| GGGTCGATGTCCATGCCCCA | Novel | NAG |
| GGGTCGTCCGCAGGATTCAG | Novel | NGC |
| GGGTGGAGCCCTCAGGCTCA | Novel | NGG |
| GGGTGTTAATTTGGAAGATC | Novel | NAG |
| GGGTTACTCTCTGAATTTTA | Novel | NGG |
| GGGTTGCGTCAGCAAACACT | Novel | NGG |
| GGGTTTAAATGTATACCCAA | Novel | NGA |
| GGGTTTACTGCTCCTGAACT | Novel | NGA |
| GGGTTTCACCCCACCGCACG | Novel | NAG |
| GGTAAATATTTGGTAACCTT | Novel | NGG |
| GGTAACCTTTGGATAAAACC | Novel | NAG |
| GGTAGGAGCTGGAGCATTCG | Novel | NGC |
| GGTAGGCTGCCTTCCTGTCT | Novel | NGC |
| GGTAGTTCCCCCTAGAAAAT | Novel | NGA |
| GGTATACATTTAAACCCTAA | Novel | NAA |
| GGTATTAAACCTTATTATCC | Novel | NGA |
| GGTATTGTGAGGATTCTTGT | Novel | NAA |
| GGTCACCATATTCTTGGGAA | Novel | NAA |
| GGTCATGGGCCATCAGCGCG | Novel | NGC |
| GGTCCCGTGCTGGTGGTTGA | Novel | NGA |
| GGTCGATGTCCATGCCCCAA | Novel | NGC |
| GGTCGGAACGGCAGACGGAG | Novel | NAG |
| GGTCGGGAAAGAATCCCAGA | Novel | NGA |
| GGTCGGTCGGAACGGCAGAC | Novel | NGA |
| GGTCGGTCGTTGACATTGCA | Novel | NAG |
| GGTCTCAATCGCCGCGTCGC | M76 | NGA |
| GGTCTCAATCGCCGCGTCGCA | M13 | NNNRRT |
| GGTCTCCATGCGACGTGCAG | M63 | NGG |
| GGTCTCTAGATGCTGGATCTT | Novel | NNNRRT |
| GGTCTGGAGCAAACATTATC | Novel | NGG |
| GGTCTGTGCCAAGTGTTTGC | Novel | NGA |
| GGTGAGGTGAACAATGCTCA | Novel | NGA |
| GGTGAGTGATTGGAGGTTGG | Novel | NGA |
| GGTGCAATTTCCGTCCGAAGG | Novel | NNNRRT |
| GGTGCGCCCCGTGGTCGGTC | Novel | NGA |
| GGTGGAAAGATTCTGCCCCA | Novel | NGC |
| GGTGGAGCCCTCAGGCTCAG | Novel | NGC |
| GGTGGGGTGAAACCCAGCCC | Novel | NAA |
| GGTGGTCGGGAAAGAATCCC | Novel | NGA |
| GGTGGTCGGGAAAGAATCCC | Novel | NNAGGA |
| GGTGGTCGGGAAAGAATCCCA | Novel | NNGRRT |
| GGTGGTCGGGAAAGAATCCCA | Novel | NNNRRT |
| GGTGGTCTCCATGCGACGTG | Novel | NAG |
| GGTGGTCTCCATGCGACGTGC | M33 | NNNRRT |
| GGTGTAAATGTATATTAGGA | Novel | NAA |
| GGTGTTAATTTGGAAGATCC | Novel | NGC |
| GGTGTTTTCCAATGAGGATT | Novel | NAA |
| GGTTACCAAATATTTACCAT | Novel | NGG |
| GGTTACTCTCTGAATTTTAT | Novel | NGG |
| GGTTATGTCATTGGAAGTTA | Novel | NGG |
| GGTTCAGGTATTGTTTACAC | Novel | NGA |
| GGTTCCACGCACGCGCTGAT | Novel | NGC |
| GGTTCCTTGAGCAGTAGTCA | Novel | NGC |
| GGTTCCTTGAGCAGTAGTCAT | Novel | NNNRRT |
| GGTTCGGCATGGTCCCGTGC | Novel | NGG |
| GGTTCGGCATGGTCCCGTGCT | Novel | NNNRRT |
| GGTTGAGGATCCTGGAATTA | Novel | NAG |
| GGTTGCGTCAGCAAACACTT | Novel | NGC |
| GGTTGGGGACTGCGAATTTT | Novel | NGC |
| GGTTTAATACCCTTATCCAA | Novel | NGG |
| GGTTTACTGCTCCTGAACTG | Novel | NAG |
| GGTTTCACCCCACCGCACGG | Novel | NGG |
| GGTTTGGTACAGCAACAGGA | Novel | NGG |
| GGTTTTATCCAAAGGTTACC | Novel | NAA |
| GTAAACAAAGGACGTCCCGC | Novel | NNAGGA |
| GTAAACAAAGGACGTCCCGCG | Novel | NNGRRT |
| GTAAACAAAGGACGTCCCGCG | Novel | NNNRRT |
| GTAAACCCTGTTCCGACTAC | Novel | NGC |
| GTAAACTGAGCCAGGAGAAA | Novel | NGG |
| GTAAAGAGAGGTGCGCCCCG | Novel | NGG |
| GTAAATAGTGTCTAGTTTGG | Novel | NAG |
| GTAAATAGTGTCTAGTTTGGA | Novel | NNNRRT |
| GTAAATATTTGGTAACCTTT | Novel | NGA |
| GTAAATGGGGCAGCAAAACC | Novel | NAA |
| GTAACACGAGAAGGGGTCCT | Novel | NGG |
| GTAACCTTTGGATAAAACCT | Novel | NGC |
| GTAAGACCTTGGGCAACATT | Novel | NGG |
| GTAAGTTGGCGAGAAAGTGA | Novel | NAG |
| GTACGAGATCTTCTAGATAC | Novel | NGC |
| GTACTAGGAGGCTGTAGGCA | Novel | NAA |
| GTACTGAAGGAAAGAAGTCA | Novel | NAA |
| GTAGAAGAATAAAGACCAGT | Novel | NAA |
| GTAGCTCCAAATTCTTTATA | Novel | NGG |
| GTAGGAGCTGGAGCATTCGGG | Novel | NNGRRT |
| GTAGGAGCTGGAGCATTCGGG | Novel | NNNRRT |
| GTAGGCATAAATTGGTCTGC | Novel | NNACCA |
| GTAGGCCCACTTACAGTTAA | Novel | NGA |
| GTAGTATGCCCTGAGCCTGA | Novel | NGG |
| GTAGTCAGTTATGTCAACAC | Novel | NAA |
| GTAGTCATGCAGGTTCGGCA | Novel | NGG |
| GTAGTCGGAACAGGGTTTAC | Novel | NGC |
| GTAGTTCCCCCTAGAAAATT | Novel | NAG |
| GTAGTTTCCGGAAGTGTTGA | Novel | NAG |
| GTATACATTTAAACCCTAAC | Novel | NAA |
| GTATACCCAAAGACAAAAGA | Novel | NAA |
| GTATCTAGAAGATCTCGTAC | Novel | NGA |
| GTATGATGTGTTCTTGTGGC | Novel | NAG |
| GTATGCATGTATTCAATCTA | Novel | NGC |
| GTATGGATCGGCAGAGGAGC | Novel | NGA |
| GTATTAAACCTTATTATCCA | Novel | NAA |
| GTATTCCCATCCCATCATCC | Novel | NGG |
| GTATTTGGTGTCTTTCGGAGT | Novel | NNGRRT |
| GTATTTGGTGTCTTTCGGAGT | Novel | NNNRRT |
| GTCAACACTAATATGGGCCT | Novel | NAA |
| GTCAATCTTCTCGAGGATTG | Novel | NGG |
| GTCACCATATTCTTGGGAAC | Novel | NAG |
| GTCATTAGTTCCCCCCAGCA | Novel | NAG |
| GTCCAAGAGTCCTCTTATGT | Novel | NAG |
| GTCCAAGGAATACTAACATT | Novel | NAG |
| GTCCACCACGAGTCTAGACTC | E16/M2 | NNNRRT |
| GTCCAGAAGAACCAACAAGA | Novel | NGA |
| GTCCATGCCCCAAAGCCACC | Novel | NAA |
| GTCCCAAGCGACCCCGAGAA | Novel | NGG |
| GTCCCGATAATGTTTGCTCC | Novel | NGA |
| GTCCCGCGCAGGATCCAGTT | Novel | NGC |
| GTCCCGTCGGCGCTGAATCC | Novel | NGC |
| GTCCGGCAGATGAGAAGGCA | Novel | NAG |
| GTCCTACTGTTCAAGCCTCC | Novel | NAG |
| GTCCTCTTATGTAAGACCTT | Novel | NGG |
| GTCCTTTGTTTACGTCCCGT | Novel | NGG |
| GTCGCAGAAGATCTCAATCT | Novel | NGG |
| GTCGGAACGGCAGACGGAGA | Novel | NGG |
| GTCGGTCGGAACGGCAGACG | Novel | NAG |
| GTCGGTCGTTGACATTGCAG | Novel | NGA |
| GTCTAACAACAGTAGTTTCC | Novel | NGA |
| GTCTAGACTCTGCGGTATTG | Novel | NGA |
| GTCTAGACTCTGCGGTATTG | Novel | NNAGGA |
| GTCTAGACTCTGCGGTATTGT | Novel | NNGRRT |
| GTCTAGACTCTGCGGTATTGT | Novel | NNNRRT |
| GTCTATAAGCTGGAGGAGTG | Novel | NGA |
| GTCTCAATCGCCGCGTCGCA | Novel | NAA |
| GTCTGCGCACCAGCACCATG | Novel | NAA |
| GTCTGGAGCAAACATTATCG | Novel | NGA |
| GTCTGGCCAGGTGTCCTTGT | Novel | NGG |
| GTCTGGCGATTGGTGGAGGC | Novel | NGG |
| GTCTGTGCCTTCTCATCTGC | Novel | NGG |
| GTCTTACATAAGAGGACTCT | Novel | NGG |
| GTCTTGGTGTAAATGTATAT | Novel | NAG |
| GTCTTTAAACAAACAGTCTT | Novel | NGA |
| GTCTTTGAAGTATGCCTCAAG | Novel | NNNRRT |
| GTCTTTGTACTAGGAGGCTG | Novel | NAG |
| GTGAAAAAGTTGCATGGTGC | Novel | NGG |
| GTGAAACCACAAGAGTTGCC | Novel | NGA |
| GTGAGAGGCCTGTATTTCCC | Novel | NGC |
| GTGAGAGGCCTGTATTTCCCT | Novel | NNNRRT |
| GTGAGGATTCTTGTCAACAA | Novel | NAA |
| GTGAGGTGAACAATGCTCAG | Novel | NAG |
| GTGATGTTCTCCATGTTCAG | Novel | NGC |
| GTGCACACGGTCCGGCAGAT | Novel | NAG |
| GTGCAGTATGGTGAGGTGAA | Novel | NAA |
| GTGCCAAGTGTTTGCTGACG | Novel | NAA |
| GTGCGAATCCACACTCCGAA | Novel | NGA |
| GTGCGCCCCGTGGTCGGTCG | Novel | NAA |
| GTGCTGCCAACTGGATCCTG | Novel | NGC |
| GTGCTGGTGGTTGAGGATCC | Novel | NGG |
| GTGGACTTCTCTCAATTTTC | Novel | NAG |
| GTGGAGACAGCGGGGTAGGC | Novel | NGC |
| GTGGAGGCAGGAGGCGGATT | Novel | NGC |
| GTGGGTCACCATATTCTTGG | Novel | NAA |
| GTGGGTCTTTTGGGTTTTGC | Novel | NGC |
| GTGGTCGGGAAAGAATCCCA | Novel | NAG |
| GTGGTCTCCATGCGACGTGC | M93 | NGA |
| GTGGTTGAGGATCCTGGAAT | Novel | NAG |
| GTGTAAATAGTGTCTAGTTT | Novel | NGA |
| GTGTAAATGTATATTAGGAA | Novel | NAG |
| GTGTGGATTCGCACTCCTCC | Novel | NGC |
| GTGTTGACATAACTGACTAC | Novel | NAG |
| GTGTTTCTCAAAGGTGGAGA | Novel | NAG |
| GTGTTTTCCAATGAGGATTA | Novel | NAG |
| GTTAATCATTACTTCCAAAC | Novel | NAG |
| GTTACCAAATATTTACCATT | Novel | NGA |
| GTTACCAATTTTCTTTTGTCT | Novel | NNGRRT |
| GTTACCAATTTTCTTTTGTCT | Novel | NNNRRT |
| GTTAGTATTCCTTGGACTCA | Novel | NAA |
| GTTATCGCTGGATGTGTCTG | Novel | NGG |
| GTTATGGGTCCTTGCCACAA | Novel | NAA |
| GTTATGTCAACACTAATATG | Novel | NGC |
| GTTATGTCATTGGAAGTTAT | Novel | NGG |
| GTTCACATTTTTTGATAATGT | Novel | NNNRRT |
| GTTCAGGTATTGTTTACACA | Novel | NAA |
| GTTCCCCACCTTATGAGTCC | Novel | NAG |
| GTTCCCCACCTTATGAGTCCA | Novel | NNGRRT |
| GTTCCCCACCTTATGAGTCCA | Novel | NNNRRT |
| GTTCCCCCTAGAAAATTGAG | Novel | NGA |
| GTTCCGCAGTATGGATCGGC | E17 | NGA |
| GTTCCGCAGTATGGATCGGC | Novel | NNAGGA |
| GTTCTTGTGGCAAGGACCCA | Novel | NAA |
| GTTGACATTGCAGAGAGTCC | Novel | NAG |
| GTTGAGGATCCTGGAATTAG | Novel | NGG |
| GTTGATAGGATAGGGGCATT | Novel | NGG |
| GTTGATAGGATAGGGGCATTT | Novel | NNNRRT |
| GTTGCATGGTGCTGGTGCGC | Novel | NGA |
| GTTGCATGGTGCTGGTGCGC | Novel | NNACCA |
| GTTGCCCAAGGTCTTACATA | Novel | NGA |
| GTTGCCCAAGGTCTTACATA | Novel | NNAGGA |
| GTTGCCGGGCAACGGGGTAA | Novel | NGG |
| GTTGGAGGACAGGAGGTTGG | Novel | NGA |
| GTTGGATCCAGCCTTCAGAG | Novel | NAA |
| GTTGGGGGAGGAGATTAGAT | Novel | NAA |
| GTTGGGGGAGGAGATTAGATT | Novel | NNNRRT |
| GTTGGTGAGTGATTGGAGGT | Novel | NGG |
| GTTGGTTCTTCTGGACTATC | Novel | NAG |
| GTTTAAAGACTGGGAGGAGT | Novel | NGG |
| GTTTAATACCCTTATCCAATG | Novel | NNNRRT |
| GTTTACACAGAAAGGCCTTG | Novel | NAA |
| GTTTACGTCCCGTCGGCGCT | Novel | NAA |
| GTTTACTAGTGCCATTTGTT | Novel | NAG |
| GTTTACTAGTGCCATTTGTTC | Novel | NNNRRT |
| GTTTACTGCTCCTGAACTGG | Novel | NGC |
| GTTTCACCCCACCGCACGGA | Novel | NGC |
| GTTTCTCAAAGGTGGAGACAG | Novel | NNGRRT |
| GTTTCTCAAAGGTGGAGACAG | Novel | NNNRRT |
| GTTTGCTCCAGACCTGCTGC | Novel | NAG |
| GTTTGGAAGTAATGATTAAC | Novel | NAG |
| GTTTGGTACAGCAACAGGAG | Novel | NGA |
| GTTTGTTTAAAGACTGGGAG | Novel | NAG |
| GTTTTCCAATGAGGATTAAAG | M15 | NNNRRT |
| GTTTTGCTCGCAGCAGGTCT | Novel | NGA |
| GTTTTGCTGCCCCATTTACA | Novel | NAA |
| TAAAACCTAGCAGGCATAAT | Novel | NAA |
| TAAAACGCCGCAGACACATC | Novel | NAG |
| TAAAACGCCGCAGACACATCC | E18 | NNNRRT |
| TAAACAAAGGACGTCCCGCG | Novel | NAG |
| TAAACCCTAACAAAACAAAG | Novel | NGA |
| TAAACCTTATTATCCAGAACA | Novel | NNNRRT |
| TAAACTGAGCCAGGAGAAAC | Novel | NGG |
| TAAAGAATTTGGAGCTACTG | Novel | NGG |
| TAAAGACAGGTACAGTAGAA | Novel | NAA |
| TAAAGACTGGGAGGAGTTGG | Novel | NGG |
| TAAAGAGAGGTGCGCCCCGTG | Novel | NNNRRT |
| TAAAGGTCTTTGTACTAGGA | Novel | NGC |
| TAAAGTTCAGGCAACTCTTG | Novel | NGG |
| TAAATGGGGCAGCAAAACCC | Novel | NAA |
| TAAATGTATACCCAAAGACA | Novel | NAA |
| TAACACCCACCCAGGTAGCT | Novel | NGA |
| TAACACGAGAAGGGGTCCTA | Novel | NGA |
| TAACAGCGGTAAAAAGGGACT | Novel | NNNRRT |
| TAACAGGCCTATTGATTGGA | Novel | NAG |
| TAACATTGAGGTTCCCGAGAT | Novel | NNNRRT |
| TAACCACATTGTGTAAATGG | Novel | NGC |
| TAACCAGGACAAGTTGGAGG | Novel | NNAGGA |
| TAACTAGATGTTCTGGATAA | Novel | NAA |
| TAACTGAAAGCCAAACAGTG | Novel | NGG |
| TAACTGACTACTAGGTCTCT | Novel | NGA |
| TAAGAGGACTCTTGGACTCTC | Novel | NNNRRT |
| TAAGCAGGCTTTCACTTTCT | Novel | NGC |
| TAAGGGAGAGGCAGTAGTCG | Novel | NAA |
| TAAGGTTTAATACCCTTATC | Novel | NAA |
| TAAGGTTTAATACCCTTATCC | Novel | NNNRRT |
| TAAGTTGGCGAGAAAGTGAA | Novel | NGC |
| TAATAAGGTTTAATACCCTTA | Novel | NNNRRT |
| TAATACCCTTATCCAATGGT | Novel | NAA |
| TAATATACCCGCCTTCCATA | Novel | NAG |
| TAATATGGGCCTAAAGTTCA | Novel | NGC |
| TAATCTCCTCCCCCAACTCCT | Novel | NNNRRT |
| TAATGAGAAAAGAAGATTGCA | Novel | NNNRRT |
| TAATGATTAACTAGATGTTC | Novel | NGG |
| TAATGCCCTTGTATGCATGTA | Novel | NNNRRT |
| TACAAACTGTTCACATTTTT | Novel | NGA |
| TACAAACTGTTCACATTTTTT | Novel | NNNRRT |
| TACACAATGTGGTTATCCTGC | M31 | NNNRRT |
| TACACCAAGACATTATCAAA | Novel | NAA |
| TACAGAGCTGAGGCGGTATC | Novel | NAG |
| TACAGGTGCAATTTCCGTCC | Novel | NAA |
| TACAGTAGAAGAATAAAGAC | Novel | NAG |
| TACATCGTATCCATGGCTGC | Novel | NAG |
| TACCAATTTTCTTTTGTCTT | Novel | NGG |
| TACCACATCATCCATATAAC | M71 | NGA |
| TACCATTGGATAAGGGTATT | Novel | NAA |
| TACCCCGCTGTCTCCACCTT | Novel | NGA |
| TACCCCGTTGCCCGGCAACGG | Novel | NNNRRT |
| TACCCGCCTTCCATAGAGTGT | Novel | NNNRRT |
| TACCGCAGAGTCTAGACTCG | M34 | NGG |
| TACCGCCTCAGCTCTGTATC | Novel | NGG |
| TACCTGAACCTTTACCCCGT | Novel | NGC |
| TACCTGGGTGGGTGTTAATT | Novel | NGG |
| TACCTGTCTTTAATCCTCAT | Novel | NGG |
| TACCTTGTTGGCGTCTGGCC | Novel | NGG |
| TACGATGTATATTTGCGGGA | Novel | NAG |
| TACTAACATTGAGGTTCCCG | E24_splice | NGA |
| TACTAGGAGGCTGTAGGCAT | Novel | NAA |
| TACTAGTGCCATTTGTTCAG | Novel | NGG |
| TACTGAAGGAAAGAAGTCAG | Novel | NAG |
| TACTGCCTCTCCCTTATCGT | Novel | NAA |
| TACTTCAAAGACTGTTTGTT | Novel | NAA |
| TACTTTCCAATCAATAGGCCT | M29 | NNNRRT |
| TAGAAAACTTCCTATTAACA | Novel | NGC |
| TAGAAGAATAAAGACCAGTA | Novel | NAG |
| TAGAAGATCTCGTACTGAAG | Novel | NAA |
| TAGACTCTGCGGTATTGTGA | Novel | NGA |
| TAGAGTATTTGGTGTCTTTC | Novel | NGA |
| TAGAGTCATTAGTTCCCCCC | Novel | NGC |
| TAGAGTGTGTAAATAGTGTC | Novel | NAG |
| TAGATGTTCTGGATAATAAGG | Novel | NNNRRT |
| TAGATTAAAGGTCTTTGTAC | Novel | NAG |
| TAGATTGAATACATGCATAC | Novel | NAG |
| TAGCAGCAGGATGAAGAGGA | Novel | NGA |
| TAGCAGCAGGATGAAGAGGAA | Novel | NNNRRT |
| TAGCCGCTTGTTTTGCTCGC | Novel | NGC |
| TAGCCGCTTGTTTTGCTCGCA | Novel | NNNRRT |
| TAGCTCCAAATTCTTTATAA | Novel | NGG |
| TAGGAAAAGATGGTGTTTTC | Novel | NAA |
| TAGGAATTTTCCGAAAGCCC | Novel | NGG |
| TAGGACCCCTTCTCGTGTTA | Novel | NAG |
| TAGGCAGAGGTGAAAAAGTTG | Novel | NNNRRT |
| TAGGCCCACTTACAGTTAAT | Novel | NAG |
| TAGGCTGCCTTCCTGTCTGG | Novel | NGA |
| TAGGGCTTTCCCCCACTGTT | Novel | NGG |
| TAGGGGCATTTGGTGGTCTA | Novel | NAA |
| TAGGGTTTAAATGTATACCC | Novel | NAA |
| TAGTACAAAGACCTTTAATC | Novel | NAA |
| TAGTATGCCCTGAGCCTGAG | Novel | NGC |
| TAGTATTCCTTGGACTCATA | Novel | NGG |
| TAGTCCAGAAGAACCAACAA | Novel | NAA |
| TAGTGTTGACATAACTGACTA | Novel | NNNRRT |
| TAGTTCCCCCTAGAAAATTG | Novel | NGA |
| TAGTTTCCGGAAGTGTTGAT | Novel | NGG |
| TAGTTTGGAAGTAATGATTAA | Novel | NNNRRT |
| TATAACGGTTTCTCTTCCAA | Novel | NAG |
| TATAACTGAAAGCCAAACAG | Novel | NGG |
| TATAAGAGAGAAACAACACA | Novel | NAG |
| TATAATATACCCGCCTTCCA | Novel | NAG |
| TATACATTTAAACCCTAACA | Novel | NAA |
| TATACCCAAAGACAAAAGAAA | M7 | NNNRRT |
| TATAGAGTATTTGGTGTCTTT | Novel | NNGRRT |
| TATAGAGTATTTGGTGTCTTT | Novel | NNNRRT |
| TATATGGATGATGTGGTATT | Novel | NGG |
| TATATTATATAAGAGAGAAA | Novel | NAA |
| TATATTTGCGGGAGAGGACAA | Novel | NNGRRT |
| TATATTTGCGGGAGAGGACAA | Novel | NNNRRT |
| TATCATCTTCCTCTTCATCC | Novel | NGC |
| TATCCATGGCTGCTAGGCTG | Novel | NGC |
| TATCCCTCCTGTTGCTGTAC | Novel | NAA |
| TATCCTATCAACACTTCCGG | Novel | NAA |
| TATCTAGAAGATCTCGTACT | Novel | NAA |
| TATCTAGAAGATCTCGTACT | Novel | NNAGGA |
| TATGATGTGTTCTTGTGGCA | Novel | NGG |
| TATGCCTCAAGGTCGGTCGT | Novel | NGA |
| TATGCCTGCTAGGTTTTATC | Novel | NAA |
| TATGCCTGCTAGGTTTTATCC | Novel | NNNRRT |
| TATGGATCGGCAGAGGAGCC | Novel | NAA |
| TATGGATGATGTGGTATTGG | M94 | NGG |
| TATGGTGACCCACAAAATGA | Novel | NGC |
| TATGGTGAGGTGAACAATGC | Novel | NNAGGA |
| TATGTAAGACCTTGGGCAACA | Novel | NNNRRT |
| TATGTATCCCTCCTGTTGCT | Novel | NNACCA |
| TATGTTGCCCGTTTGTCCTC | Novel | NAA |
| TATTAACAGGCCTATTGATT | Novel | NGA |
| TATTAACAGGCCTATTGATTG | Novel | NNNRRT |
| TATTAGGAAAAGATGGTGTTT | Novel | NNNRRT |
| TATTATCCAGAACATCTAGT | Novel | NAA |
| TATTCCCATCCCATCATCCT | Novel | NGG |
| TATTCCTTGGACTCATAAGG | Novel | NGG |
| TATTCTTCTACTGTACCTGTC | Novel | NNNRRT |
| TATTGATTGGAAAGTATGTCA | Novel | NNGRRT |
| TATTGATTGGAAAGTATGTCA | Novel | NNNRRT |
| TATTGGGGGCCAAGTCTGTA | Novel | NAG |
| TATTTACACACTCTATGGAA | Novel | NGC |
| TATTTACACACTCTATGGAAG | Novel | NNGRRT |
| TATTTACACACTCTATGGAAG | Novel | NNNRRT |
| TATTTCCCTGCTGGTGGCTC | Novel | NAG |
| TATTTGCGGGAGAGGACAAC | Novel | NGA |
| TATTTGGTAACCTTTGGATA | Novel | NAA |
| TCAACACTAATATGGGCCTA | Novel | NAG |
| TCAACGAATTGTGGGTCTTT | M61 | NGG |
| TCAAGATGCTGTACAGACTT | Novel | NGC |
| TCAAGGTCGGTCGTTGACAT | Novel | NGC |
| TCAATAGGCCTGTTAATAGG | Novel | NAG |
| TCAATCCCAACAAGGACACC | M52 | NGG |
| TCAATCTTCTCGAGGATTGG | Novel | NGA |
| TCACCAAACTCTGCAAGATCC | Novel | NNGRRT |
| TCACCAAACTCTGCAAGATCC | Novel | NNNRRT |
| TCACCATACTGCACTCAGGC | Novel | NAG |
| TCACCATACTGCACTCAGGCA | Novel | NNNRRT |
| TCACCATATTCTTGGGAACA | Novel | NGA |
| TCACCTCACCATACTGCACT | Novel | NAG |
| TCACCTCTGCACGTCGCATG | Novel | NAG |
| TCACTCTGGGATCTTGCAGAG | Novel | NNNRRT |
| TCACTTTCTCGCCAACTTAC | Novel | NAG |
| TCAGAAGGCAAAAACGAGAG | Novel | NAA |
| TCAGCCCGTTTCTCCTGGCT | Novel | NAG |
| TCAGGCAAGCAATTCTTTGC | M41 | NGG |
| TCAGGCTCAGGGCATACTAC | Novel | NAA |
| TCAGGGCATACTACAAACTT | Novel | NGC |
| TCAGGTATTGTTTACACAGA | Novel | NAG |
| TCAGTTTACTAGTGCCATTTG | Novel | NNNRRT |
| TCATCCATATAACTGAAAGC | Novel | NAA |
| TCATTAGTTCCCCCCAGCAA | Novel | NGA |
| TCCAAATTCTTTATAAGGGT | Novel | NGA |
| TCCAACTGGTGGTCGGGAAA | Novel | NAA |
| TCCAACTTGTCCTGGTTATCG | Novel | NNGRRT |
| TCCAACTTGTCCTGGTTATCG | Novel | NNNRRT |
| TCCAAGAGTCCTCTTATGTA | Novel | NGA |
| TCCAAGGAATACTAACATTG | M46 | NGG |
| TCCAATCAATAGGCCTGTTA | Novel | NNAGGA |
| TCCACACTCCGAAAGACACC | Novel | NAA |
| TCCACCAATCGCCAGACAGG | Novel | NAG |
| TCCACCACGAGTCTAGACTC | Novel | NGC |
| TCCACCCCAAAAGGCCTCCG | Novel | NGC |
| TCCAGCATCTAGAGACCTAG | Novel | NAG |
| TCCAGCCTTCAGAGCAAACA | Novel | NAG |
| TCCAGTTGGCAGCACAGCCT | Novel | NGC |
| TCCATGCCCCAAAGCCACCC | Novel | NAG |
| TCCATGCGACGTGCAGAGGT | Novel | NAA |
| TCCCAACAAGGACACCTGGC | Novel | NAG |
| TCCCAAGAATATGGTGACCCA | M20 | NNNRRT |
| TCCCAGAGGATTGCTGGTGG | Novel | NAA |
| TCCCATAGGAATTTTCCGAA | Novel | NGC |
| TCCCATCATCCTGGGCTTTC | Novel | NGA |
| TCCCATCATCCTGGGCTTTCG | Novel | NNNRRT |
| TCCCCAATCCTCGAGAAGAT | M85 | NGA |
| TCCCCAATCCTCGAGAAGATT | M24 | NNNRRT |
| TCCCCACCTTATGAGTCCAA | Novel | NGA |
| TCCCCCTAGAAAATTGAGAG | Novel | NAG |
| TCCCGACCACCAGTTGGATC | Novel | NAG |
| TCCCTGCTGGTGGCTCCAGT | Novel | NNAGGA |
| TCCCTTATCGTCAATCTTCT | Novel | NGA |
| TCCCTTATCGTCAATCTTCT | Novel | NNAGGA |
| TCCCTTATCGTCAATCTTCTC | Novel | NNGRRT |
| TCCCTTATCGTCAATCTTCTC | Novel | NNNRRT |
| TCCCTTTTTACCGCTGTTAC | Novel | NAA |
| TCCGAAAGCCCAGGATGATG | Novel | NGA |
| TCCGAAGGTTTGGTACAGCA | Novel | NNAGGA |
| TCCGCAGGATTCAGCGCCGA | Novel | NGG |
| TCCGCAGTATGGATCGGCAG | E19 | NGG |
| TCCGGAAGTGTTGATAGGAT | Novel | NGG |
| TCCGGCAGATGAGAAGGCAC | Novel | NGA |
| TCCGTCCGAAGGTTTGGTAC | Novel | NGC |
| TCCTAATATACATTTACACC | Novel | NAG |
| TCCTACCTTGTTGGCGTCTGG | Novel | NNNRRT |
| TCCTACTGTTCAAGCCTCCA | Novel | NGC |
| TCCTAGCCGCTTGTTTTGCT | Novel | NGC |
| TCCTAGTACAAAGACCTTTAA | Novel | NNNRRT |
| TCCTCCAGCTTATAGACCAC | Novel | NAA |
| TCCTCGAGAAGATTGACGAT | Novel | NAG |
| TCCTCTAATTCCAGGATCCT | Novel | NAA |
| TCCTCTAATTCCAGGATCCT | Novel | NNACCA |
| TCCTCTGCCGATCCATACTG | E20 | NGG |
| TCCTCTTATGTAAGACCTTG | Novel | NGC |
| TCCTCTTCATCCTGCTGCTA | Novel | NGC |
| TCCTGAACTGGAGCCACCAG | Novel | NAG |
| TCCTGCCTCCACCAATCGCC | Novel | NGA |
| TCCTGCGGACGACCCTTCTC | Novel | NGG |
| TCCTGGAATTAGAGGACAAA | Novel | NGG |
| TCCTGTCCTCCAACTTGTCC | Novel | NGG |
| TCCTGTCTGGCGATTGGTGG | Novel | NGG |
| TCCTTCAGTACGAGATCTTC | Novel | NAG |
| TCCTTGAGCAGTAGTCATGC | Novel | NGG |
| TCCTTGGACTCATAAGGTGG | Novel | NGA |
| TCCTTTGTTTACGTCCCGTC | Novel | NGC |
| TCGACCCTTATAAAGAATTT | Novel | NGA |
| TCGAGAAGATTGACGATAAG | Novel | NGA |
| TCGCAGAAGATCTCAATCTC | Novel | NGG |
| TCGCAGACGAAGGTCTCAAT | Novel | NGC |
| TCGGAAAATTCCTATGGGAG | Novel | NGG |
| TCGGAACGGCAGACGGAGAA | Novel | NGG |
| TCGGCATGGTCCCGTGCTGG | Novel | NGG |
| TCGGCGCTGAATCCTGCGGA | Novel | NGA |
| TCGGTCGGAACGGCAGACGG | Novel | NGA |
| TCGGTCGTTGACATTGCAGA | Novel | NAG |
| TCGTACTGAAGGAAAGAAGT | Novel | NAG |
| TCGTCAATCTTCTCGAGGAT | Novel | NGG |
| TCGTCCGCAGGATTCAGCGC | Novel | NGA |
| TCGTTGACATACTTTCCAAT | Novel | NAA |
| TCTAACAACAGTAGTTTCCG | Novel | NAA |
| TCTAAGGCTTCCCGATACAG | Novel | NGC |
| TCTAATTCCAGGATCCTCAA | Novel | NNACCA |
| TCTAGAAGATCTCGTACTGA | Novel | NGG |
| TCTAGACTCTGCGGTATTGT | Novel | NAG |
| TCTAGTTAATCATTACTTCC | Novel | NAA |
| TCTAGTTTGGAAGTAATGAT | Novel | NAA |
| TCTATAACGGTTTCTCTTCC | Novel | NAA |
| TCTATAAGCTGGAGGAGTGC | Novel | NAA |
| TCTCAAAGGTGGAGACAGCG | Novel | NGG |
| TCTCAATCGCCGCGTCGCAG | Novel | NAG |
| TCTCACTCTGGGATCTTGCA | Novel | NAG |
| TCTCCGTCTGCCGTTCCGAC | Novel | NGA |
| TCTCCGTCTGCCGTTCCGAC | Novel | NNACCA |
| TCTCCTCCCCCAACTCCTCC | Novel | NAG |
| TCTCCTGAGCATTGTTCACC | Novel | NNACCA |
| TCTCTAGATGCTGGATCTTC | Novel | NAA |
| TCTCTCTTATATAATATACC | Novel | NGC |
| TCTCTTCCAAAAGTGAGACA | Novel | NGA |
| TCTGACTTCTTTCCTTCAGTA | Novel | NNNRRT |
| TCTGCAAGATCCCAGAGTGA | Novel | NAG |
| TCTGCCGTTCCGACCGACCA | Novel | NGG |
| TCTGGACTATCAAGGTATGT | Novel | NGC |
| TCTGGAGCAAACATTATCGGG | Novel | NNNRRT |
| TCTGGCCAGGTGTCCTTGTT | Novel | NGG |
| TCTGGCGATTGGTGGAGGCA | Novel | NGA |
| TCTGTGCCTTCTCATCTGCC | Novel | NGA |
| TCTTACATAAGAGGACTCTT | Novel | NGA |
| TCTTCCAAAAGTGAGACAAG | Novel | NAA |
| TCTTCTACTGTACCTGTCTT | Novel | NAA |
| TCTTCTGCGACGCGGCGATT | Novel | NAG |
| TCTTCTTTTCTCATTAACTG | Novel | NAA |
| TCTTGGACTCTCTGCAATGT | Novel | NAA |
| TCTTGGTGTAAATGTATATT | Novel | NGG |
| TCTTGTCTCACTTTTGGAAG | Novel | NGA |
| TCTTGTTCCCAAGAATATGG | Novel | NGA |
| TCTTTAAACAAACAGTCTTT | Novel | NAA |
| TCTTTAATCCTCATTGGAAA | Novel | NNACCA |
| TCTTTCCACCAGCAATCCTC | Novel | NGG |
| TCTTTGCTGGGGGGAACTAA | Novel | NGA |
| TCTTTGTACTAGGAGGCTGT | Novel | NGG |
| TCTTTGTTTTGTTAGGGTTT | Novel | NAA |
| TCTTTTCCTAATATACATTT | Novel | NNACCA |
| TGAAACCACAAGAGTTGCCT | Novel | NAA |
| TGAAAGCCAAACAGTGGGGG | Novel | NAA |
| TGAACAAGAGATGATTAGGC | Novel | NGA |
| TGAACAGTAGGACATGAACA | Novel | NGA |
| TGAACAGTTTGTAGGCCCACT | M17 | NNNRRT |
| TGAACATGGAGAACATCACA | Novel | NNAGGA |
| TGAACATGGAGAACATCACAT | Novel | NNGRRT |
| TGAACATGGAGAACATCACAT | Novel | NNNRRT |
| TGAACCTTTACCCCGTTGCC | Novel | NGG |
| TGAACTGGAGCCACCAGCAG | Novel | NGA |
| TGAAGAGGAAGATGATAAAA | Novel | NGC |
| TGAAGGCTGGATCCAACTGG | Novel | NGG |
| TGACATAACCCATAAAATTC | Novel | NGA |
| TGACATAACCCATAAAATTCA | Novel | NNGRRT |
| TGACATAACCCATAAAATTCA | Novel | NNNRRT |
| TGACATACTTTCCAATCAAT | Novel | NGG |
| TGACATTGCAGAGAGTCCAA | Novel | NAG |
| TGACCAAGCCCCAGCCAGTG | Novel | NGG |
| TGACGATAAGGGAGAGGCAG | Novel | NAG |
| TGACGCAACCCCCACTGGCT | Novel | NGG |
| TGACTACTAGGTCTCTAGATG | Novel | NNGRRT |
| TGACTACTAGGTCTCTAGATG | Novel | NNNRRT |
| TGACTTCTTTCCTTCAGTAC | Novel | NAG |
| TGAGAAAAGAAGATTGCAAT | Novel | NGA |
| TGAGACCTTCGTCTGCGAGG | Novel | NGA |
| TGAGATCTTCTGCGACGCGG | Novel | NGA |
| TGAGCCAGGAGAAACGGGCT | Novel | NAG |
| TGAGCCTGAGGGCTCCACCC | Novel | NAA |
| TGAGGATGAGTGTTTCTCAA | Novel | NGG |
| TGAGGATTCTTGTCAACAAG | Novel | NAA |
| TGAGGCATAGCAGCAGGATG | Novel | NAG |
| TGAGGTGAACAATGCTCAGG | Novel | NGA |
| TGAGTCCCTTTTTACCGCTG | Novel | NNACCA |
| TGAGTGCAGTATGGTGAGGT | Novel | NAA |
| TGAGTGCAGTATGGTGAGGTG | Novel | NNNRRT |
| TGAGTGTTTCTCAAAGGTGG | Novel | NGA |
| TGATAGTCCAGAAGAACCAA | Novel | NAA |
| TGATGGGATGGGAATACAGG | Novel | NGC |
| TGATGTTCTCCATGTTCAGCG | Novel | NNGRRT |
| TGATGTTCTCCATGTTCAGCG | Novel | NNNRRT |
| TGATTGGAAAGTATGTCAAC | Novel | NAA |
| TGATTGGAGGTTGGGGACTG | Novel | NGA |
| TGCAAGATCCCAGAGTGAGA | Novel | NGC |
| TGCAATTGATTATGCCTGCT | M48 | NGG |
| TGCACACGGTCCGGCAGATG | Novel | NGA |
| TGCATACAAGGGCATTAACG | Novel | NAG |
| TGCATGTATTCAATCTAAGC | Novel | NGG |
| TGCCAACTGGATCCTGCGCG | M189 | NGA |
| TGCCACAAGAACACATCATA | Novel | NAA |
| TGCCCAAGGTCTTACATAAG | Novel | NGG |
| TGCCCGTTTGTCCTCTAATT | Novel | NNAGGA |
| TGCCCGTTTGTCCTCTAATTC | Novel | NNGRRT |
| TGCCCGTTTGTCCTCTAATTC | Novel | NNNRRT |
| TGCCCTTGTATGCATGTATT | Novel | NAA |
| TGCCGAACCTGCATGACTAC | Novel | NGC |
| TGCCGTTCCGACCGACCACG | Novel | NGG |
| TGCCTACAGCCTCCTAGTAC | Novel | NAA |
| TGCCTCCACCAATCGCCAGA | Novel | NAG |
| TGCCTGAGTGCAGTATGGTG | Novel | NGG |
| TGCCTGCTAGGTTTTATCCA | Novel | NAG |
| TGCGACGTGCAGAGGTGAAG | Novel | NGA |
| TGCGAGCAAAACAAGCGGCT | Novel | NGG |
| TGCGGTGGGGTGAAACCCAGC | Novel | NNGRRT |
| TGCGGTGGGGTGAAACCCAGC | Novel | NNNRRT |
| TGCTAGGCTGTGCTGCCAAC | Novel | NGG |
| TGCTAGGTTTTATCCAAAGG | Novel | NNACCA |
| TGCTCCAGACCTGCTGCGAG | Novel | NAA |
| TGCTCCAGCTCCTACCTTGT | M54 | NGG |
| TGCTCCTGAACTGGAGCCAC | Novel | NAG |
| TGCTCGCAGCAGGTCTGGAG | Novel | NAA |
| TGCTGGCAAAGTTTGTAGTA | Novel | NGC |
| TGCTGGTGGCTCCAGTTCAG | Novel | NAG |
| TGCTGGTGGCTCCAGTTCAGG | Novel | NNNRRT |
| TGCTGGTGGTTGAGGATCCT | Novel | NGA |
| TGCTGTACAGACTTGGCCCC | Novel | NAA |
| TGCTGTACCAAACCTTCGGA | Novel | NGG |
| TGCTGTACCAAACCTTCGGAC | M26 | NNNRRT |
| TGCTGTAGATCTTGTTCCCA | Novel | NGA |
| TGGAAAACACCATCTTTTCC | Novel | NAA |
| TGGAAAGTATGTCAACGAATT | Novel | NNGRRT |
| TGGAAAGTATGTCAACGAATT | Novel | NNNRRT |
| TGGAACCTTTTCGGCTCCTC | Novel | NGC |
| TGGAACCTTTTCGGCTCCTCT | Novel | NNNRRT |
| TGGAAGGCGGGTATATTATA | Novel | NAA |
| TGGACATCGACCCTTATAAA | Novel | NAA |
| TGGACTCTCTGCAATGTCAA | Novel | NGA |
| TGGACTTCTCTCAATTTTCT | E21 | NGG |
| TGGATAAAACCTAGCAGGCA | Novel | NAA |
| TGGATACGATGTATATTTGC | Novel | NGG |
| TGGATCCAACTGGTGGTCGG | Novel | NAA |
| TGGATCGGCAGAGGAGCCGA | Novel | NAA |
| TGGATGATGTGGTATTGGGGG | Novel | NNNRRT |
| TGGATTTGCTGTGTTTGCTC | Novel | NGA |
| TGGCAAGGACCCATAACTTC | Novel | NAA |
| TGGCACTAGTAAACTGAGCC | Novel | NGG |
| TGGCAGCACAGCCTAGCAGCC | Novel | NNGRRT |
| TGGCAGCACAGCCTAGCAGCC | Novel | NNNRRT |
| TGGCCAAAATTCGCAGTCCC | Novel | NAA |
| TGGCCAGACGCCAACAAGGT | Novel | NGG |
| TGGCGATTGGTGGAGGCAGG | Novel | NGG |
| TGGCTCCAGTTCAGGAGCAG | Novel | NAA |
| TGGCTGCTAGGCTGTGCTGC | Novel | NAA |
| TGGCTTTGGGGCATGGACAT | Novel | NGA |
| TGGGAACAAGATCTACAGCA | M49 | NGG |
| TGGGACTTCAATCCCAACAA | Novel | NGA |
| TGGGATCTTGCAGAGTTTGG | Novel | NGA |
| TGGGATTCTTTCCCGACCAC | Novel | NAG |
| TGGGATTGAAGTCCCAATCT | Novel | NGA |
| TGGGCCATCAGCGCGTGCGT | Novel | NGA |
| TGGGGACCCTGCGCTGAACA | Novel | NGG |
| TGGGGACTGCGAATTTTGGC | Novel | NAA |
| TGGGGCAGAATCTTTCCACC | Novel | NGC |
| TGGGGGAGGAGATTAGATTA | Novel | NAG |
| TGGGGGGAACTAATGACTCT | Novel | NGC |
| TGGGGTGGAGCCCTCAGGCT | Novel | NAG |
| TGGGGTTACTCTCTGAATTTT | Novel | NNGRRT |
| TGGGGTTACTCTCTGAATTTT | Novel | NNNRRT |
| TGGGTGGGTGTTAATTTGGA | Novel | NGA |
| TGGGTTATGTCATTGGAAGTT | Novel | NNGRRT |
| TGGGTTATGTCATTGGAAGTT | Novel | NNNRRT |
| TGGGTTTCACCCCACCGCAC | Novel | NGA |
| TGGGTTTTGCTGCCCCATTTA | Novel | NNNRRT |
| TGGTCCCGTGCTGGTGGTTG | Novel | NGG |
| TGGTCGGGAAAGAATCCCAG | Novel | NGG |
| TGGTCGGTCGGAACGGCAGA | Novel | NGG |
| TGGTCTATAAGCTGGAGGAG | Novel | NGC |
| TGGTCTATAAGCTGGAGGAGT | Novel | NNGRRT |
| TGGTCTATAAGCTGGAGGAGT | Novel | NNNRRT |
| TGGTCTCCATGCGACGTGCA | Novel | NAG |
| TGGTCTGCGCACCAGCACCA | Novel | NGC |
| TGGTGACCCACAAAATGAGG | Novel | NGC |
| TGGTGAGGTGAACAATGCTC | Novel | NGG |
| TGGTGAGTGATTGGAGGTTG | Novel | NGG |
| TGGTGGCTCCAGTTCAGGAG | Novel | NAG |
| TGGTGGTCGGGAAAGAATCC | Novel | NAG |
| TGGTGGTCTATAAGCTGGAG | Novel | NAG |
| TGGTGTAAATGTATATTAGG | Novel | NAA |
| TGGTGTAAATGTATATTAGGA | Novel | NNNRRT |
| TGGTGTTTTCCAATGAGGAT | Novel | NAA |
| TGGTTATCGCTGGATGTGTC | Novel | NGC |
| TGGTTGAGGATCCTGGAATT | Novel | NGA |
| TGGTTGAGGATCCTGGAATT | Novel | NNAGGA |
| TGTAAATAGTGTCTAGTTTG | Novel | NAA |
| TGTAAATGTATATTAGGAAA | Novel | NGA |
| TGTAAATGTATATTAGGAAAA | Novel | NNNRRT |
| TGTAACACGAGAAGGGGTCC | Novel | NAG |
| TGTAACACGAGAAGGGGTCCT | Novel | NNGRRT |
| TGTAACACGAGAAGGGGTCCT | Novel | NNNRRT |
| TGTAAGTTGGCGAGAAAGTG | Novel | NAA |
| TGTACCAAACCTTCGGACGG | Novel | NAA |
| TGTAGATCTTGTTCCCAAGAA | Novel | NNNRRT |
| TGTAGGCATAAATTGGTCTG | Novel | NGC |
| TGTAGTATGCCCTGAGCCTG | Novel | NGG |
| TGTATACCCAAAGACAAAAG | Novel | NAA |
| TGTATATTTGCGGGAGAGGA | Novel | NAA |
| TGTATGATGTGTTCTTGTGG | Novel | NAA |
| TGTATGATGTGTTCTTGTGG | Novel | NNAGGA |
| TGTATGCATGTATTCAATCT | Novel | NAG |
| TGTCAACACTAATATGGGCC | Novel | NAA |
| TGTCAACGAATTGTGGGTCTT | M30 | NNGRRT |
| TGTCAACGAATTGTGGGTCTT | Novel | NNNRRT |
| TGTCCTACTGTTCAAGCCTC | Novel | NAA |
| TGTCCTTGTTGGGATTGAAGT | Novel | NNNRRT |
| TGTCTGGCGATTGGTGGAGG | Novel | NAG |
| TGTCTTGGTGTAAATGTATA | Novel | NNAGGA |
| TGTCTTTAATCCTCATTGGA | Novel | NAA |
| TGTCTTTCGGAGTGTGGATT | Novel | NGC |
| TGTGAGGATTCTTGTCAACA | Novel | NGA |
| TGTGCACTTCGCTTCACCTC | Novel | NGC |
| TGTGCCTTGGGTGGCTTTGG | Novel | NGC |
| TGTGGAGTTACTCTCGTTTT | Novel | NGC |
| TGTGGGTCACCATATTCTTG | Novel | NGA |
| TGTGTAAATAGTGTCTAGTT | Novel | NGG |
| TGTGTAAATAGTGTCTAGTTT | Novel | NNNRRT |
| TGTGTCTTGGCCAAAATTCG | Novel | NAG |
| TGTGTTGTTTCTCTCTTATA | Novel | NAA |
| TGTTAATAGGAAGTTTTCTA | Novel | NAA |
| TGTTAGTATTCCTTGGACTCA | Novel | NNNRRT |
| TGTTCATGTCCTACTGTTCA | Novel | NGC |
| TGTTCTCCATGTTCAGCGCA | Novel | NGG |
| TGTTGACATAACTGACTACT | Novel | NGG |
| TGTTGCCCAAGGTCTTACAT | Novel | NAG |
| TGTTGCTGTACCAAACCTTC | Novel | NGA |
| TGTTGGGATTGAAGTCCCAAT | Novel | NNGRRT |
| TGTTGGGATTGAAGTCCCAAT | Novel | NNNRRT |
| TGTTGGTTCTTCTGGACTAT | Novel | NAA |
| TGTTTACGTCCCGTCGGCGC | Novel | NGA |
| TGTTTCTCAAAGGTGGAGAC | Novel | NGC |
| TGTTTGCTCCAGACCTGCTG | Novel | NGA |
| TGTTTGGCTTTCAGTTATAT | Novel | NGA |
| TGTTTGGCTTTCAGTTATATG | Novel | NNNRRT |
| TGTTTGTTTAAAGACTGGGA | Novel | NGA |
| TGTTTTAGAAAACTTCCTAT | Novel | NAA |
| TGTTTTCCAATGAGGATTAA | M79 | NGA |
| TGTTTTGCTCGCAGCAGGTC | Novel | NGG |
| TTAAACCCTAACAAAACAAA | Novel | NAG |
| TTAAAGACAGGTACAGTAGA | Novel | NGA |
| TTAAAGACTGGGAGGAGTTG | Novel | NGG |
| TTAAAGGTCTTTGTACTAGG | Novel | NGG |
| TTAAATGTATACCCAAAGAC | Novel | NAA |
| TTAACACCCACCCAGGTAGC | Novel | NAG |
| TTAACAGGCCTATTGATTGG | Novel | NAA |
| TTAACTAGATGTTCTGGATAA | Novel | NNNRRT |
| TTAACTGTAAGTGGGCCTAC | Novel | NAA |
| TTAATACCCTTATCCAATGG | Novel | NAA |
| TTAATCATTACTTCCAAACT | Novel | NGA |
| TTAATGAGAAAAGAAGATTG | Novel | NAA |
| TTACACACTCTATGGAAGGC | Novel | NGG |
| TTACACCAAGACATTATCAA | Novel | NAA |
| TTACAGTTAATGAGAAAAGA | Novel | NGA |
| TTACCATTGGATAAGGGTAT | Novel | NAA |
| TTACCCCGTTGCCCGGCAAC | Novel | NGC |
| TTACGCGGACTCCCCGTCTG | Novel | NGC |
| TTACTCTCGTTTTTGCCTTC | Novel | NGA |
| TTACTGCTCCTGAACTGGAG | Novel | NNACCA |
| TTAGAAAACTTCCTATTAAC | Novel | NGG |
| TTAGATTAAAGGTCTTTGTA | Novel | NNAGGA |
| TTAGATTGAATACATGCATA | Novel | NAA |
| TTAGGCAGAGGTGAAAAAGT | Novel | NGC |
| TTAGGGTTTAAATGTATACC | Novel | NAA |
| TTAGTATTCCTTGGACTCAT | Novel | NAG |
| TTATATGGATGATGTGGTAT | Novel | NGG |
| TTATCAGTCCCGATAATGTT | Novel | NGC |
| TTATCGCTGGATGTGTCTGC | Novel | NGC |
| TTCAAAGACTGTTTGTTTAA | Novel | NGA |
| TTCAAGCCTCCAAGCTGTGCC | E22/M9 | NNGRRT |
| TTCAAGCCTCCAAGCTGTGCC | E22 | NNNRRT |
| TTCACCTCTGCACGTCGCAT | Novel | NGA |
| TTCACTTTCTCGCCAACTTA | Novel | NAA |
| TTCAGAGCAAACACAGCAAAT | M23 | NNNRRT |
| TTCAGGTATTGTTTACACAG | Novel | NAA |
| TTCCAAATTAACACCCACCC | Novel | NGG |
| TTCCAATGACATAACCCATA | Novel | NAA |
| TTCCAATGAGGATTAAAGAC | M47 | NGG |
| TTCCAGGATCCTCAACCACC | Novel | NGC |
| TTCCCCACCTTATGAGTCCA | Novel | NGG |
| TTCCCCCACTGTTTGGCTTT | Novel | NAG |
| TTCCCCCTAGAAAATTGAGA | Novel | NAA |
| TTCCCGAGATTGAGATCTTC | Novel | NGC |
| TTCCCGATACAGAGCTGAGG | Novel | NGG |
| TTCCGAAAGCCCAGGATGAT | Novel | NGG |
| TTCCGCAGTATGGATCGGCA | Novel | NAG |
| TTCCGGAAACTACTGTTGTT | Novel | NGA |
| TTCCGGAAGTGTTGATAGGA | Novel | NAG |
| TTCCGTCCGAAGGTTTGGTA | Novel | NAG |
| TTCCTAATATACATTTACAC | Novel | NAA |
| TTCCTATGGGAGTGGGCCTC | Novel | NGC |
| TTCCTGTCTGGCGATTGGTG | Novel | NAG |
| TTCCTTGAGCAGTAGTCATG | Novel | NAG |
| TTCCTTGGACTCATAAGGTG | Novel | NGG |
| TTCGCACTCCTCCAGCTTAT | Novel | NGA |
| TTCGCACTCCTCCAGCTTAT | Novel | NNACCA |
| TTCGCTTCACCTCTGCACGT | Novel | NGC |
| TTCTCAAAGGTGGAGACAGC | Novel | NGG |
| TTCTCATCTGCCGGACCGTG | Novel | NGC |
| TTCTCGAGGATTGGGGACCC | Novel | NGC |
| TTCTCTCAATTTTCTAGGGG | Novel | NAA |
| TTCTCTTCCAAAAGTGAGAC | Novel | NAG |
| TTCTCTTCCAAAAGTGAGACA | Novel | NNNRRT |
| TTCTTGGGAACAAGATCTAC | Novel | NGC |
| TTCTTGTCTCACTTTTGGAA | Novel | NAG |
| TTCTTTCCCGACCACCAGTT | Novel | NGA |
| TTGAACAGTAGGACATGAAC | Novel | NAG |
| TTGAACAGTAGGACATGAACA | Novel | NNNRRT |
| TTGAAGTCCCAATCTGGATT | Novel | NGC |
| TTGACATACTTTCCAATCAA | Novel | NAG |
| TTGACATTGCAGAGAGTCCA | Novel | NGA |
| TTGAGAAACACTCATCCTCA | Novel | NGC |
| TTGAGGATCCTGGAATTAGA | Novel | NGA |
| TTGATTGGAAAGTATGTCAA | Novel | NGA |
| TTGCAATCTTCTTTTCTCAT | Novel | NAA |
| TTGCAATTGATTATGCCTGC | Novel | NAG |
| TTGCATGGTGCTGGTGCGCAG | Novel | NNNRRT |
| TTGCCAGCAAATCCGCCTCC | Novel | NGC |
| TTGCCCAAGGTCTTACATAA | Novel | NAG |
| TTGCCTGAACTTTAGGCCCAT | Novel | NNNRRT |
| TTGCCTGAGTGCAGTATGGT | Novel | NAG |
| TTGCTCTGAAGGCTGGATCCA | Novel | NNNRRT |
| TTGCTGACGCAACCCCCACT | Novel | NGC |
| TTGCTGTGTTTGCTCTGAAGG | Novel | NNGRRT |
| TTGCTGTGTTTGCTCTGAAGG | Novel | NNNRRT |
| TTGCTTGCCTGAGTGCAGTA | Novel | NGG |
| TTGGAAGAGAAACCGTTATA | Novel | NAG |
| TTGGAAGATCCAGCATCTAG | Novel | NGA |
| TTGGAGGACAGGAGGTTGGT | Novel | NAG |
| TTGGAGGACAGGAGGTTGGTG | Novel | NNNRRT |
| TTGGATCCAGCCTTCAGAGC | Novel | NAA |
| TTGGCGAGAAAGTGAAAGCC | Novel | NGC |
| TTGGCTTTCAGTTATATGGA | Novel | NGA |
| TTGGGACTTCAATCCCAACA | Novel | NGG |
| TTGGGATTGAAGTCCCAATC | Novel | NGG |
| TTGGGGGAGGAGATTAGATT | Novel | NAA |
| TTGGGTATACATTTAAACCC | Novel | NAA |
| TTGGTAACAGCGGTAAAAAG | Novel | NGA |
| TTGGTGAGTGATTGGAGGTT | Novel | NGG |
| TTGGTGGTCTATAAGCTGGA | Novel | NGA |
| TTGGTGTAAATGTATATTAG | Novel | NAA |
| TTGGTTCTTCTGGACTATCA | Novel | NGG |
| TTGTAAGTTGGCGAGAAAGT | Novel | NAA |
| TTGTACTAGGAGGCTGTAGGC | Novel | NNNRRT |
| TTGTAGTATGCCCTGAGCCT | Novel | NAG |
| TTGTATGCATGTATTCAATC | Novel | NAA |
| TTGTCTCACTTTTGGAAGAG | Novel | NAA |
| TTGTCTTTGGGTATACATTT | Novel | NAA |
| TTGTGAGGATTCTTGTCAAC | Novel | NAG |
| TTGTGGCAAGGACCCATAACT | Novel | NNNRRT |
| TTGTGGGTCACCATATTCTT | Novel | NGG |
| TTGTTCATGTCCTACTGTTC | Novel | NAG |
| TTGTTGGTTCTTCTGGACTAT | Novel | NNNRRT |
| TTGTTTACACAGAAAGGCCTT | Novel | NNNRRT |
| TTTAAACCCTAACAAAACAA | Novel | NGA |
| TTTAAAGACTGGGAGGAGTT | Novel | NGG |
| TTTAAATGTATACCCAAAGA | Novel | NAA |
| TTTAATCTAATCTCCTCCCC | Novel | NAA |
| TTTACACAATGTGGTTATCC | Novel | NGC |
| TTTACACACTCTATGGAAGG | Novel | NGG |
| TTTACACAGAAAGGCCTTGT | Novel | NAG |
| TTTACACCAAGACATTATCA | Novel | NAA |
| TTTACCCCGTTGCCCGGCAA | Novel | NGG |
| TTTAGAAAACTTCCTATTAA | Novel | NAG |
| TTTATAAGGGTCGATGTCCA | Novel | NGC |
| TTTATGGGTTATGTCATTGG | Novel | NAG |
| TTTCAGTTATATGGATGATG | Novel | NGG |
| TTTCCAATCAATAGGCCTGT | Novel | NAA |
| TTTCCAATGAGGATTAAAGA | Novel | NAG |
| TTTCCACCAGCAATCCTCTG | M80 | NGA |
| TTTCCGAAAGCCCAGGATGA | Novel | NGG |
| TTTCCTTCAGTACGAGATCTT | Novel | NNNRRT |
| TTTCTCAAAGGTGGAGACAG | Novel | NGG |
| TTTCTCATTAACTGTAAGTG | Novel | NGC |
| TTTCTCCTGGCTCAGTTTAC | Novel | NAG |
| TTTCTCTTCCAAAAGTGAGA | Novel | NAA |
| TTTCTGTGTAAACAATACCT | Novel | NAA |
| TTTCTTGTCTCACTTTTGGA | Novel | NGA |
| TTTCTTGTTGACAAGAATCCT | Novel | NNNRRT |
| TTTGAAGTATGCCTCAAGGT | Novel | NGG |
| TTTGAGAAACACTCATCCTC | Novel | NGG |
| TTTGCCTTCTGACTTCTTTCC | Novel | NNNRRT |
| TTTGCTCCAGACCTGCTGCG | Novel | NGC |
| TTTGCTCGCAGCAGGTCTGG | Novel | NGC |
| TTTGCTCTGAAGGCTGGATC | Novel | NAA |
| TTTGCTGACGCAACCCCCAC | E23_whb | NGG |
| TTTGCTGCCCCATTTACACAA | Novel | NNNRRT |
| TTTGCTGTGTTTGCTCTGAA | Novel | NGC |
| TTTGGAAGAGAAACCGTTAT | Novel | NGA |
| TTTGGAAGATCCAGCATCTA | Novel | NAG |
| TTTGGAAGTAATGATTAACT | Novel | NGA |
| TTTGGTGGTCTATAAGCTGG | Novel | NGG |
| TTTGGTGTCTTTCGGAGTGT | Novel | NGA |
| TTTGTAGGCCCACTTACAGT | Novel | NAA |
| TTTGTAGTATGCCCTGAGCC | Novel | NGA |
| TTTGTATGATGTGTTCTTGT | Novel | NGC |
| TTTGTCTTTGGGTATACATT | Novel | NAA |
| TTTGTGGGTCACCATATTCT | Novel | NGG |
| TTTGTTTACGTCCCGTCGGCG | Novel | NNGRRT |
| TTTGTTTACGTCCCGTCGGCG | Novel | NNNRRT |
| TTTTATGGGTTATGTCATTG | Novel | NAA |
| TTTTCCGAAAGCCCAGGATGA | Novel | NNGRRT |
| TTTTCCGAAAGCCCAGGATGA | Novel | NNNRRT |
| TTTTCTCATTAACTGTAAGT | Novel | NGG |
| TTTTGATAATGTCTTGGTGT | Novel | NAA |
| TTTTGCTCGCAGCAGGTCTG | Novel | NAG |
| TTTTGGAAGAGAAACCGTTA | Novel | NAG |
| TTTTGTATGATGTGTTCTTG | Novel | NGG |
| TTTTTGATAATGTCTTGGTG | Novel | NAA |

### Example 5: Establishment and Validation of HBV-Infected Primary Human Hepatocyte (PHH) System

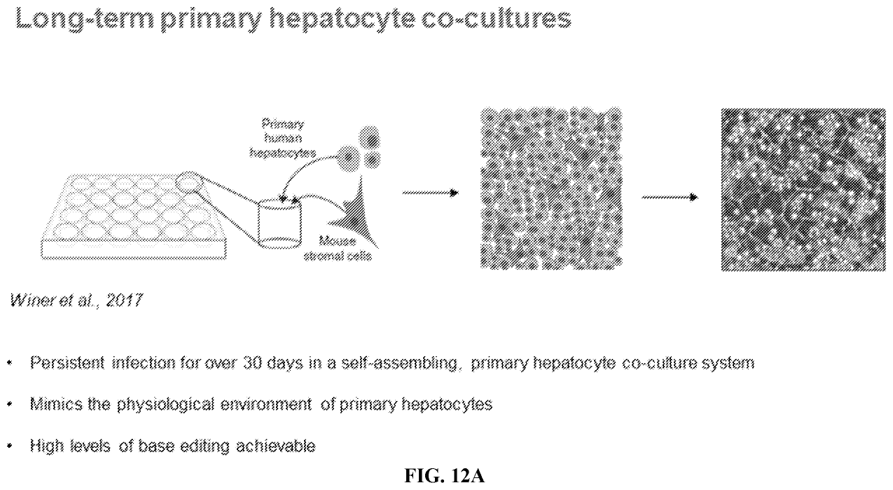

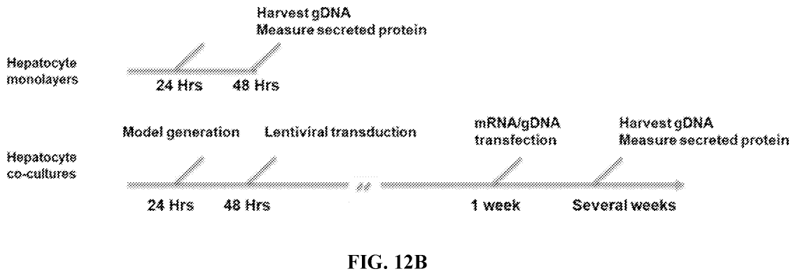

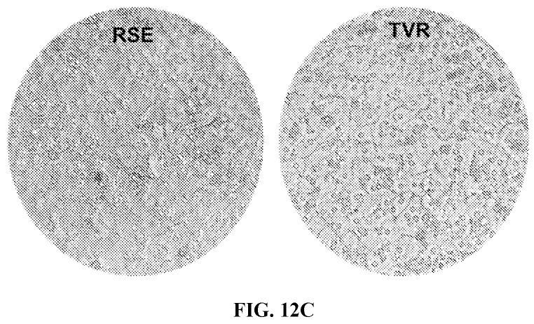

A system comprising HBV-infected primary human hepatocytes (PHHs) was established by persistent HBV infection for over 30 days in a self-assembling, primary hepatocyte co-culture system (FIG. 12A). To generate the co-cultures system, primary human hepatocytes (PHH) (BioIVT) were plated at 350 k cells per well in a Collagen-Type I coated 24-well plate (Corning, 354408), and incubated at 37° C., 5% C02 to generate an adherent cell monolayer. 4 hrs post plating, plated hepatocytes were washed with CP medium (BioIVT) to remove any unadhered cells. 3T3-J2 murine embryonic fibroblasts (Kerafast (distributed from Howard Green (Harvard), Boston) were seeded at a ratio of 95% hepatocyte:5% fibroblast per well and cultured at 37° C., 5% C02 for an additional 12 hours to form co-cultures. Culture medium was replaced every 2 days (500 μL per well) for continued maintenance. Shown in FIG. 12C are images of transduced primary hepatocytes from two human hepatocyte donors, RSE and TVR, isolated from two different human livers. They were isolated, cryopreserved, and distributed for sale for research purposes by BioIVT (Maryland, US).

On the second day, post hepatocyte co-culture formation, lentivirus was added at an MOI 500 dropwise into culture medium per well. Co-cultures were transduced for 16 hours, prior to changing media for fresh CP medium (BioIVT). Culture medium was replaced every 2 days (500 μL per well) for continued maintenance. Protein expression occurs over 7 days period in hepatic co-cultures. At day 7 post transduction, co-cultures were transfected using lipofection based reagents co-formulated with gRNA and base-editor mRNA. Cells were lysed and harvested for gDNA 48 hours post transfection. FIG. 12B provides a timeline of the in vitro transduction schedule in either hepatocyte monolayers or hepatocyte co-cultures showing representative time points. Importantly, PHH cultures not treated with polyethylene glycol (PEG) during infection tend to have very low percentages of HBV infected PHH cells.

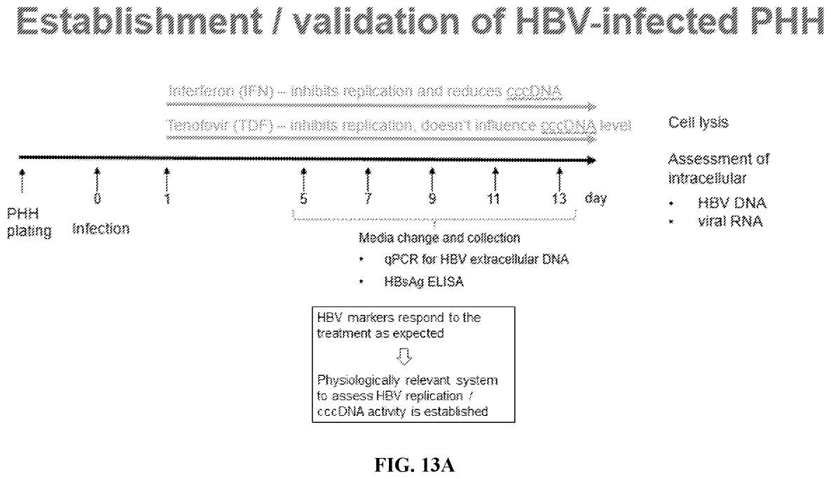

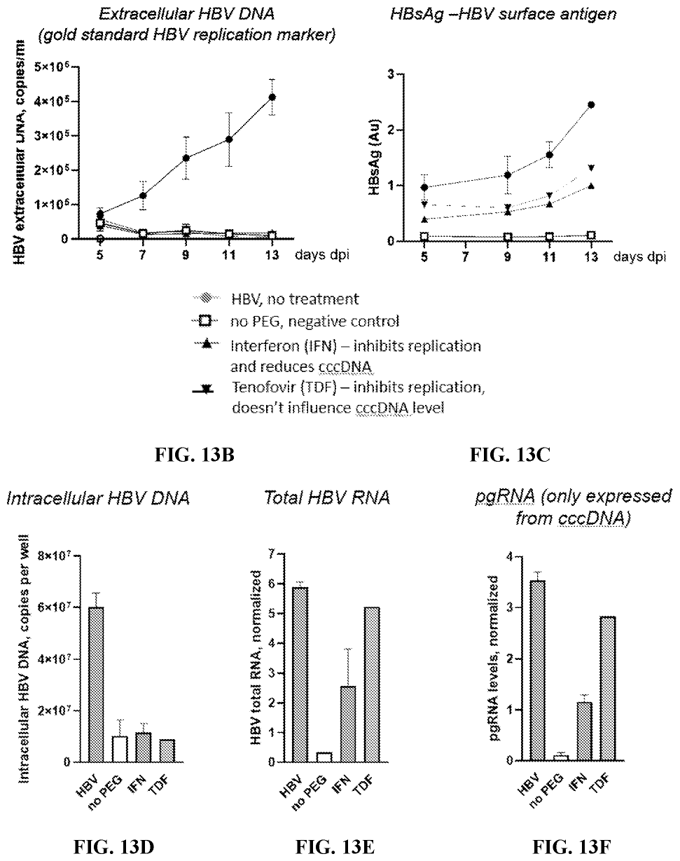

To determine if the system recapitulates the physiological environment of primary hepatocytes, HBV-infected and uninfected cultured PHH cells were treated with interferon, which inhibits replication and reduces cccDNA, or tenofovir, which inhibits replication, but does not reduce cccDNA levels, and HBV markers were measured (FIG. 13A). The infected cells, as gauged by HBV markers, respond to treatment as expected. Referring to FIG. 13B, extracellular HBV DNA, the gold standard HBV replication marker, was significantly reduced compared to untreated HBV infected control cells by day 7 post infection. The amounts of extracellular HBV DNA detected in the treated PHH cultures were similar to the amount observed in negative control cell cultures. Additionally, the treated and control cells showed near baseline levels of extracellular HBV DNA between 5 and 13 days post-infection, but the concentration of extracellular HBV DNA in the untreated HBV-infected cell cultures increased during the entire observation period (i.e., 5-13 days post infection).

Other markers associated with HBV infection include HBV surface antigen (HBsAg), intracellular HBV DNA, total HBV RNA, and pre-genomic RNA (pgRNA). Cultures treated with interferon or tenofovir have lower levels of HBV surface antigen (HBsAg), intracellular HBV DNA, total HBV RNA, and pre-genomic RNA (pgRNA) relative to negative controls (FIGS. 13C, 13D, 13E, and 13F, respectively). These results show that the HBV markers respond to treatment as expected. It also establishes that the PHH system is physiologically relevant and useful to assess HBV replication and cccDNA activity.

### Example 6: Transfection with BE4 and gRNAs Leads to a Decrease in HBV Markers Levels in HBV Infected PHH

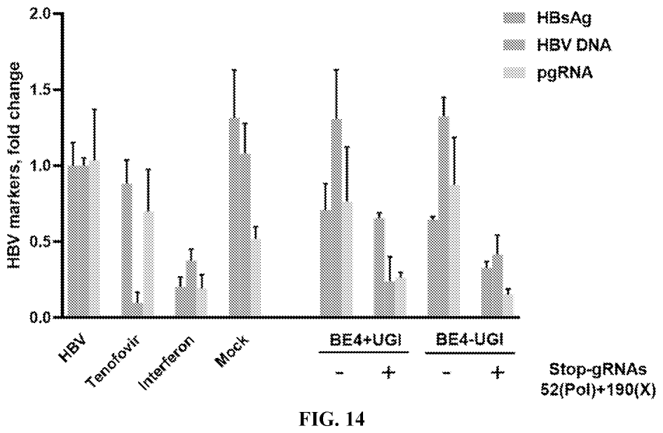

The efficacy of base editing the HBV genome in PHH cells using base editors and guide RNAs of the present invention was assessed using the system described above. Specifically, cells were transfected with a construct encoding a BE4 base editor (either with or without a UGI domain) and gRNAs. The M52 and M190 guide RNAs were chosen. These gRNAs direct the base editor to the pol and X gene regions, respectively, of the HBV genome and facilitate base changes that result in premature stop codons in these genes. Referring to FIG. 14, cells treated with a BE4 (having no UGI domain) and the HBV-targeting gRNAs consistently led to a significant reduction of all tested HBV marker levels, similar to what was observed with interferon treatment. The BE4 base editor without the UGI domain performs better than BE4 with the UGI domain. Without being bound by theory, omitting the UGI domain makes C->U deamination susceptible to uracil glycosylase, which damages HBV cccDNA, thus promoting its degradation.

### Example 7: Screen Based on HBsAg Levels (Surrogate Marker of cccDNA) in HBV-PHH Identifies Functional gRNAs

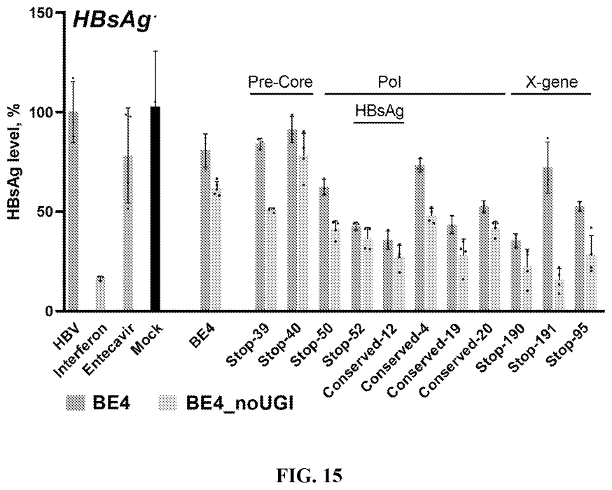

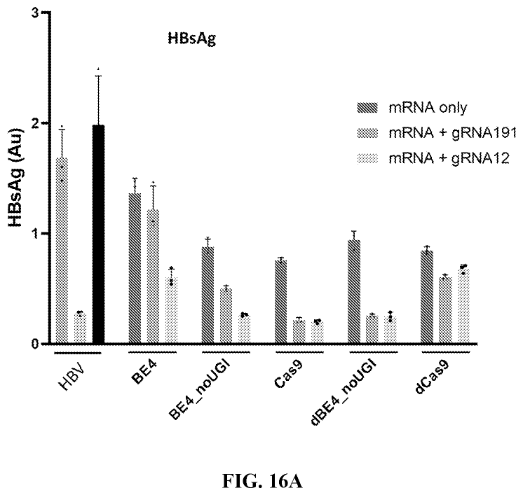

Lead functional guide RNAs were identified using the system described in Example 5. Candidate guide RNAs were introduced into HBV-infected PHHs along with a BE4 base editor and the level of HBsAg was determined. The HBsAg levels observed in cells treated with functional guide RNAs were lower than those observed in untreated cells (FIG. 15), with several approaching the levels observed in interferon treated cells. HBsAg levels in cells treated with functional guideRNAs that target the pre-core, pol, and X genes are shown in FIG. 15. BE4 base editors without UGI domains performed better, as evidenced by lower HBsAg levels, than BE4 base editors with UGI domains when combined with an HBV-targeting gRNA. The best gRNAs (gRNA191, targeting the X gene, and gRNA12, which targets the pol gene) were chosen based on the results of this screen. Levels of HBsAg (a surrogate marker for cccDNA) in cells treated with gRNA191 and BE4_noUGI were comparable levels of HBsAg resulting from interferon treatment.

### Example 8: Mechanistic Aspects of Base Editing Action on HBV

HBV infected PHH cells were transfected with mRNAs encoding a BE4 base editor, BE4 base editor lacking a UGI domain, a BE4 base editor lacking nickase activity (i.e., “dead”) and also lacking a UGI domain, Cas9, and a dead Cas9, each alone or in combination with gRNA 191 or 12. gRNA 191 targets the base editor to introduce a premature stop codon in the X gene, and gRNA 12 targets the base editor to a conserved region of the HBV Pol/S genes.

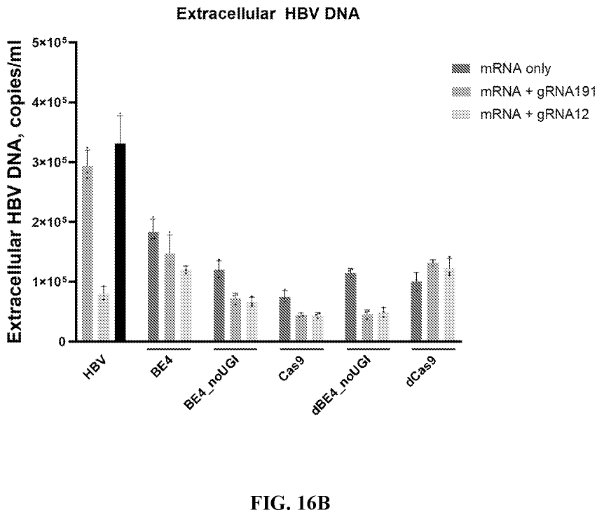

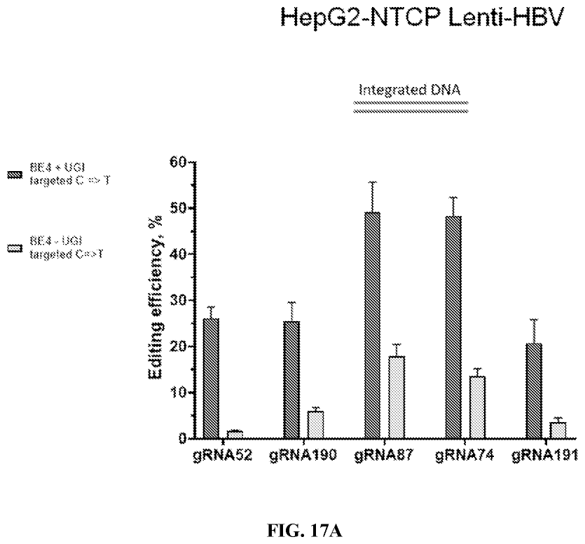

HBsAg and HBV extracellular DNA, markers of HBV, were significantly reduced in response to transfection with the base editors and gRNAs (FIGS. 16A and 16B). HBsAg levels are normalized to the untreated HBV infected cells (referred to as HBV). Nuclease activity was necessary for Cas9 activity, but was not required for base editing activity. Interestingly, introduction of Cas9 mRNA only reduced viral parameters without guide RNAs. Without being bound by theory, mRNA can induce a cellular immune response, which contributes to HBV inhibition. While HBV can exist episomally, it can also integrate into a host cell's genome. BE4 base editors lacking a UGI domain administered with gRNA showed comparable activity to Cas9 administered with gRNA. A base editor lacking nickase activity (e.g., dead BE4) can be advantageous as it reduces the potential for generating genomic double-stranded breaks if an abasic site is formed by the activity of uracil glycosylase on the converted C→U (see FIG. 3B). Without being bound by theory, to treat chronic Hepatitis B, a complex approach may be advantageous involving: 1) target cccDNA; 2) inhibit HBsAg synthesis (including from integrated HBV DNA); and 3) stimulate the immune system. These features can be found in the reagents used for base editing.

### Example 9: Comparison of Base Editing in HBV-Lenti-HepG2 and HBV Infected PHH

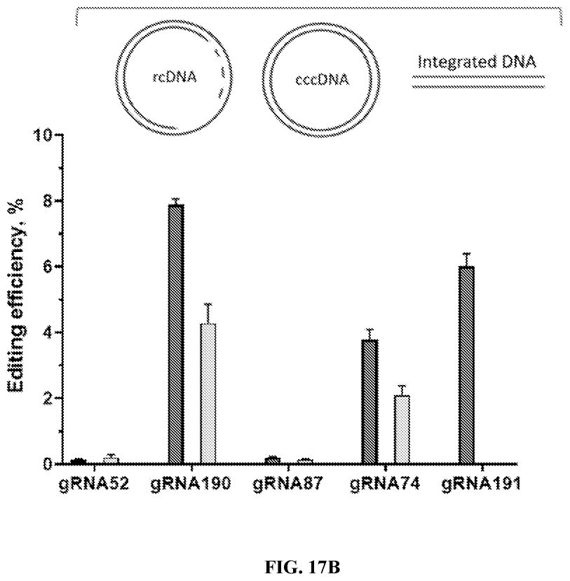

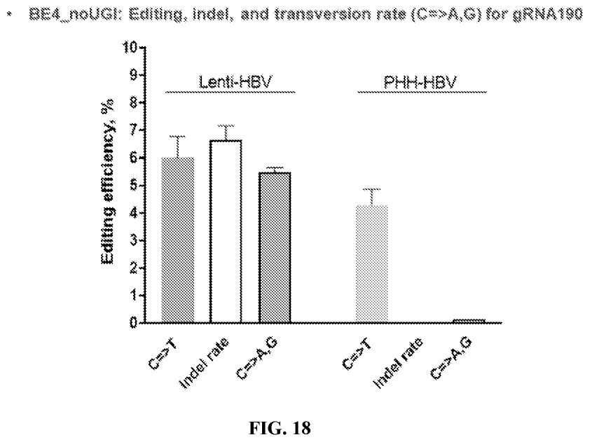

Base editing efficiencies were compared in HEPG2-NTCP Lenti-HBV and HBV-infected PHH cells. Significantly lower editing rates were observed in HBV infected PHH cells compared to HEPG2-NTCP Lenti-HBV cells (FIGS. 17A and 17B). Additionally, different patterns of gRNA efficacy were observed for the two different cell types. The HEPG2-NTCP Lenti-HBV cells showed higher base editing efficiency compared to PHH-HBV cells when using gRNA190 (FIG. 18). Interestingly, no indels or transversions were observed in the PHH-HBV cells (FIG. 18). As expected, a significant number of indel and transversions were observed in the Lenti-HBV cells edited with gRNA190 and BE4_noUGI. That there were no indels or transversions in the PHH-HBV with BE4_noUGI indicates a different fate of the edited cccDNA compared to integrated HBV DNA.

### Example 10 Primary Hepatocyte Co-Culture (PHH) Infected with HBV Virus as a Clinically Relevant System for Assessing Anti-Viral Activity of Base Editing Reagents

Experiments were performed to determine whether the base editing approach using HBV-infected primary human hepatocyte (PHH) cultures described herein was more efficient in reducing several different viral parameters compared to the activity of the known HBV antiviral drug (entecavir) and/or other controls.

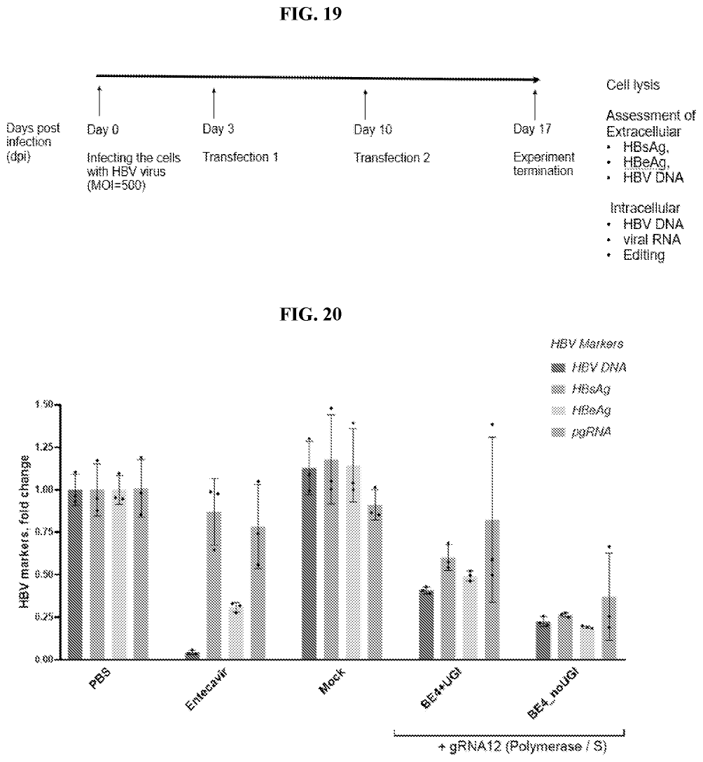

The experiments employed the approach using the HBV-infected PHH culture system and transfection of the PHH with components (base editors) of the base editor system as depicted in FIG. 19. Primary hepatocytes (PHH) co-cultures were infected with HBV in order to test antiviral efficacy of the base editors. The base editing reagents or components (base editor mRNA and synthetic gRNA) were transfected via lipofection twice over the course of two weeks. The first transfection was performed 3 days after infection to allow for complete formation of viral covalently closed circular DNA (cccDNA) at the time of the transfection. Extracellular parameters (HBsAg, HBeAg, and HBV DNA) were monitored over the course of the experiment and intracellular parameters (HBV DNA, viral RNA, and editing) were monitored at the end of the experiment. Briefly, on Day 0, PHH were infected with HBV (MOI=500). On Day 3 after HBV infection, the cells were transfected with base editing components, e.g., a BE and gRNA, or control(s). On Day 10, PHHs were subjected to another round of transfection. At the termination of the experiment, e.g., Day 17 or longer, e.g., Day 25, the PHH were lysed and extracellular amounts of HBsAg, HBeAg and HBV DNA were assessed. Also assessed were intracellular amounts of HBV DNA, viral RNA and the efficacy of base editing by the components of the base editor system versus control(s). Transfection details: RNA format, 800 ng total RNA per well in a 24 well plate (600 ng base editor+200 ng gRNA) transfected via lipofection (lipofectamin messengerMax, Thermofisher Scientific) according to the vendor's protocol.

To determine which step(s) of the HBV life cycle was/were targeted using the base editing reagents described herein, four (4) different viral parameters (HBsAg, HBeAg, HBV DNA, and pregenomic RNA (pgRNA), which is generated from cccDNA, were assessed. Pregenomic RNA (pgRNA) generated from cccDNA plays an important role in viral genome amplification and replication. Hepatic pgRNA and cccDNA expression levels indicate viral persistence and replication activity in infected cells.

HBV-infected PHH were transfected with the base editors BE4 or BE4_noUGI and gRNA, i.e., gRNA12, which targets intersection of the HBV Polymerase and S genes, in a 14-day PHH co-culture experiment, (FIG. 19), which included the common HBV antiviral drug entecavir as a comparator. Entecavir inactivates viral polymerase, thereby inhibiting replication, but does not interfere with cccDNA activity. Based on its use in the art, entecavir treatment reduces HBV DNA, however, it does not influence the expression of HBsAg and pgRNA. This result indicates that the established system is clinically relevant for assessing the efficacy of HBV antiviral reagents. The findings from this experiment demonstrated that, compared to entecavir, treatment of HBV-infected PHH with BE4_noUGI and gRNA12 resulted in the reduction of all 4 of the HBV marker parameters assessed, namely, HBV DNA, HBsAg, HBeAg and pgRNA, thus indicating that base editing using the components described herein inhibited HBV replication and disrupted cccDNA activity. (FIG. 20). In particular, the base editor BE4_noUGI+gRNA provided a greater reduction of all HBV markers compared with base editor BE4_UGI and with entecavir. (FIG. 20).

### Example 11 Base Editing is Effective in Inhibiting HBV and Reducing HBV Viral Parameters Compared to a Known HBV Antiviral Drug (Entecavir)

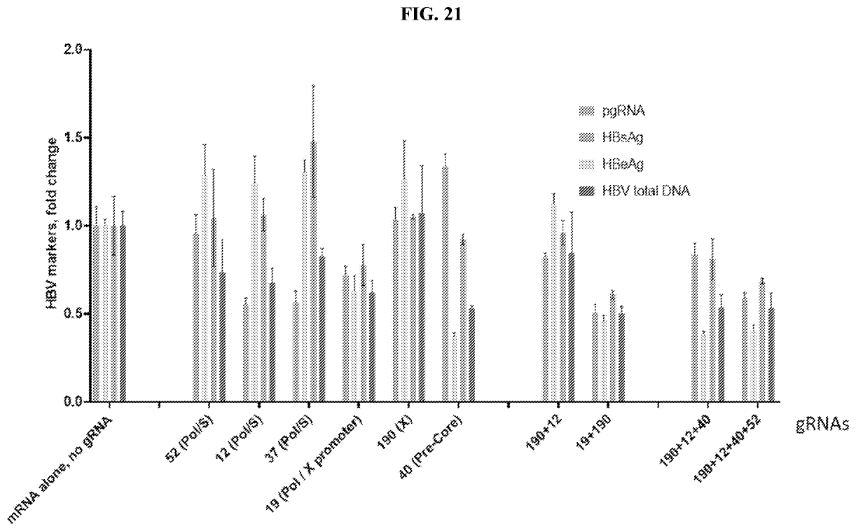

To assess whether a combination of different gRNAs (multiplexing gRNAs) in HBV-infected PHH cultures would lead to improved HBV inhibition, a comprehensive experiment comparing single gRNAs with a combination of up to 4 gRNAs targeting different regions of HBV cccDNA was performed. The experiment was carried out for both BE4 and BE4_noUGI base editors, and the results were assessed for the 4 viral parameters (HBsAg, HBeAg, HBV total DNA, pgRNA) at the termination of the experiment. Consistent with the previous results, transfection of the PHH with individual gRNAs and BE4 resulted in a small but significant reduction of particular viral parameters; the level of inhibition was dependent on the gRNA. For all of the parameters assessed, BE+gRNA19 performed slightly better than other gRNAs that were tested. A small improvement in HBV inhibition was detected using gRNA multiplexing (gRNA19+gRNA190) and BE, and a combination of BE plus four gRNAs (190+12+40+52) comparatively performed best in inhibiting HBV (FIGS. 21, 22, 23 and 24).

Table 26 presents gRNAs and corresponding polynucleotide sequences as used in the experiments described in herein (e.g., FIGS. 21-27). In Table 26, a, c, g, u: 2′-O-methyl residues; s: phosphorothioate; A, C, G, U: RNA nucleobase residues.

TABLE 26

| | gRNA | |
|---|---|---|
| Guide | Identifier | Sequence |
| CTGCCAACTGGATCCTGCGC | M191 | 5′ csusgs CCAACUGGAUCCUGCGC GUUUUAGAGC UAGAAAUAGC |
| | (gRNA191) | AAGUUAAAAU AAGGCUAGUC CGUUAUCAAC UUGAAAAAGU GGCACCGAGU |
| | | CGGUGCUsususu-3′ |
| GCTGCCAACTGGATCCTGCG | M190 | 5′ gscsus GCCAACUGGAUCCUGCG GUUUUAGAGC UAGAAAUAGC |
| | (gRNA190) | AAGUUAAAAU AAGGCUAGUC CGUUAUCAAC UUGAAAAAGU GGCACCGAGU |
| | | CGGUGCUsususu-3′ |
| GAAAGCCCAGGATGATGGGA | M37 | 5′ gsasas AGCCCAGGAUGAUGGGA GUUUUAGAGC UAGAAAUAGC |
| | (gRNA37) | AAGUUAAAAU AAGGCUAGUC CGUUAUCAAC UUGAAAAAGU GGCACCGAGU |
| | | CGGUGCUsususu-3′ |
| TCCGCAGTATGGATCGGCAG | E19 | 5′ uscscs GCAGUAUGGAUCGGCAG GUUUUAGAGC UAGAAAUAGC |
| | (gRNA19) | AAGUUAAAAU AAGGCUAGUC CGUUAUCAAC UUGAAAAAGU GGCACCGAGU |
| | | CGGUGCUsususu-3′ |
| GACTTCTCTCAATTTTCTAG | E12 | 5′ gsascs UUCUCUCAAUUUUCUAG GUUUUAGAGC UAGAAAUAGC |
| | (gRNA12) | AAGUUAAAAU AAGGCUAGUC CGUUAUCAAC UUGAAAAAGU GGCACCGAGU |
| | | CGGUGCUsususu-3′ |
| TCAATCCCAACAAGGACACC | M52 | 5′ uscsas AUCCCAACAAGGACACC GUUUUAGAGC UAGAAAUAGC |
| | (gRNA52) | AAGUUAAAAU AAGGCUAGUC CGUUAUCAAC UUGAAAAAGU GGCACCGAGU |
| | | CGGUGCUsususu-3′ |
| TCCTCTGCCGATCCATACTG | E20 | 5′ uscscs UCUGCCGAUCCAUACUG GUUUUAGAGC UAGAAAUAGC |
| | (gRNA20) | AAGUUAAAAU AAGGCUAGUC CGUUAUCAAC UUGAAAAAGU GGCACCGAGU |
| | | CGGUGCUsususu-3′ |
| CCATGCCCCAAAGCCACCCA | M40 | 5′ cscsas TGCCCCAAAGCCACCCA GUUUUAGAGC UAGAAAUAGC AAGUUAAAAU |
| | (gRNA40) | AAGGCUAGUC CGUUAUCAAC UUGAAAAAGU GGCACCGAGU CGGUGCUsususu- |
| | | 3′ |
| TATGGATGATGTGGTATTGG | M94 | 5′ usasus GGATGATGTGGTATTGG GUUUUAGAGC UAGAAAUAGC |
| | (gRNA94) | AAGUUAAAAU AAGGCUAGUC CGUUAUCAAC UUGAAAAAGU GGCACCGAGU |
| | | CGGUGCUsususu-3′ |

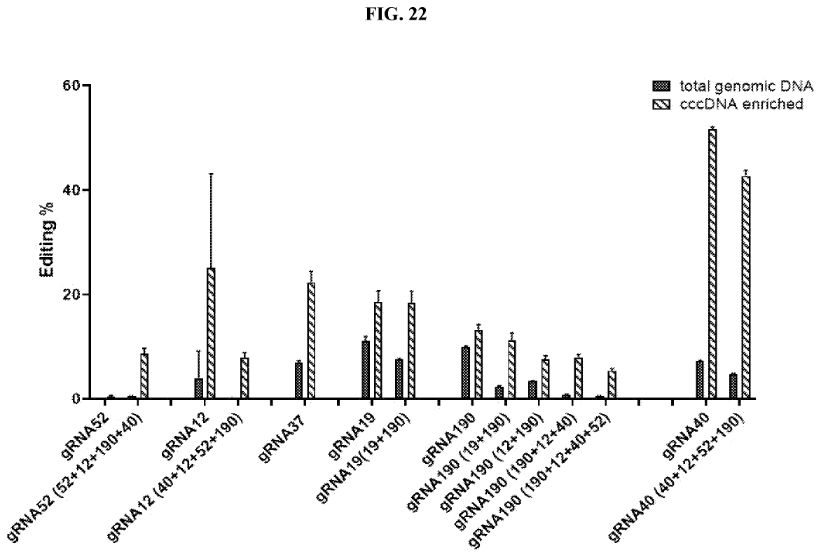

FIG. 22 shows the results of Next Generation Sequencing (NGS) performed on total DNA purified from HBV infected PHH and on the same samples enriched for cccDNA through ExoI/ExoIII digestion. FIG. 22 shows the results of base editing using BE4 at particular HBV target sites using different conditions (either individual gRNAs or a combination of gRNAs). Significantly increased base editing (increased % Editing) was observed for HBV cccDNA-enriched samples, which indicated successful cccDNA editing. The finding of a lower % Editing in total DNA (genomic plus viral DNA) purified from HBV-PHH is indicative of an inability of the edited cccDNA to propagate into a replication-competent viral particle.

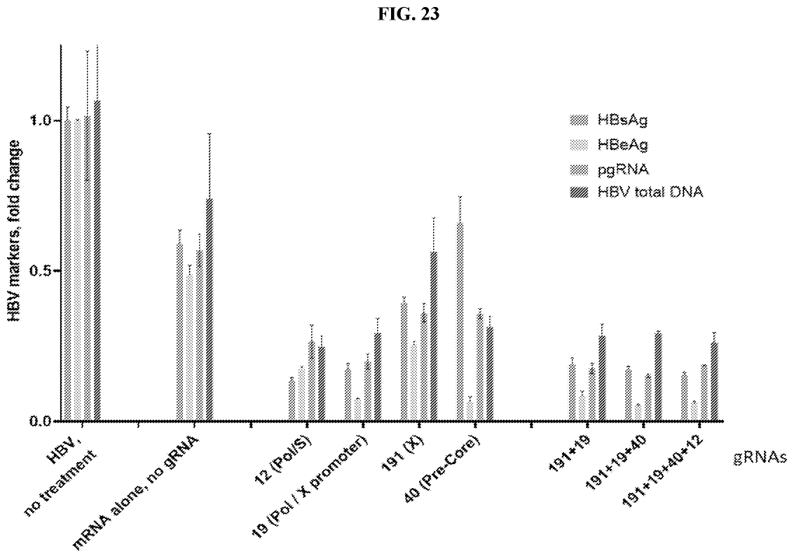

Transfection with the base editor BE4_noUGI and different gRNAs resulted in greater inhibition of the four viral parameters compared to BE4 (i.e., BE4-UGI), a result which was consistent with the previous experiments. (FIG. 23). gRNA19 was found to perform best in inhibiting HBV compared with the 4 gRNAs (gRNA12, gRNA19, gRNA191, and gRNA40) tested in the experiment. Transfection with BE4_noUGI and gRNA19 resulted in a robust antiviral response and resulted in a reduction of all tested viral parameters (HBsAg, HBeAg, pgRNA, and HBV total DNA). No improvement in antiviral efficacy was detected using BE4_noUGI with gRNA multiplexing. Transfection with BE4_noUGI and gRNA19 resulted in the same levels of reduction of viral parameters compared to those using BE4_noUGI with a combination of gRNAs. (FIG. 23).

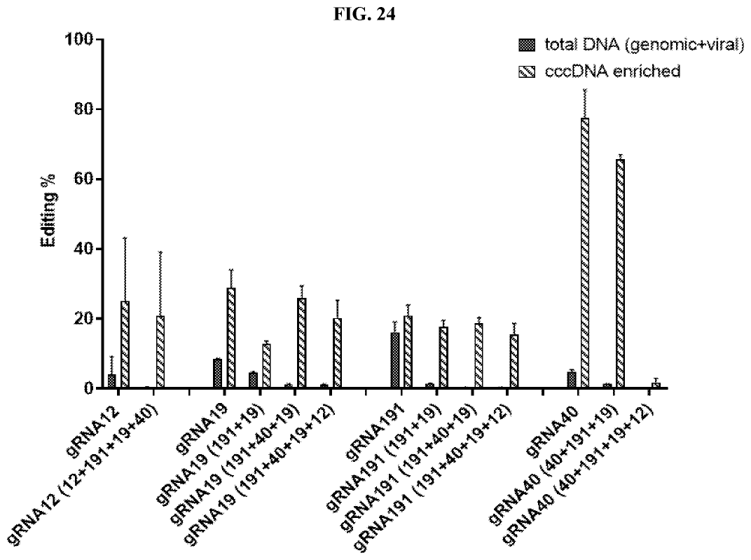

NGS sequencing was also performed on the total DNA purified from HBV infected PHH and on the same samples enriched for cccDNA through ExoI/ExoIII digestion. FIG. 24 shows base editing using BE4_noUGI on particular HBV target sites for different conditions (individual or combination of gRNAs). Significantly increased base editing (increased % Editing) was observed for HBV cccDNA-enriched samples, which indicated successful cccDNA editing. The finding of a lower % Editing in total DNA (genomic plus viral DNA) purified from HBV-PHH is indicative of an inability of the edited cccDNA to propagate into a replication-competent viral particle. (FIG. 24).

### Example 12 A Base Editor without Nickase Activity is Effective in Reducing HBV Parameters of Infection

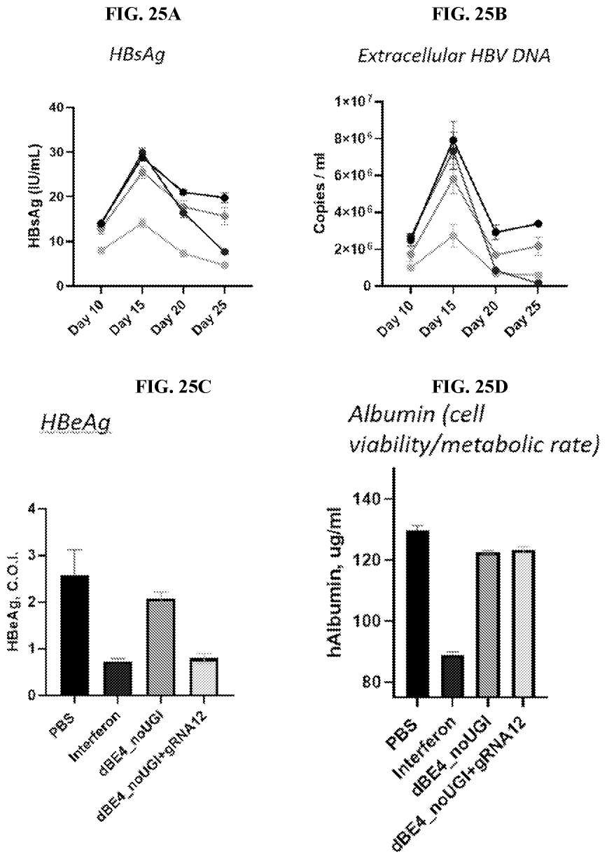

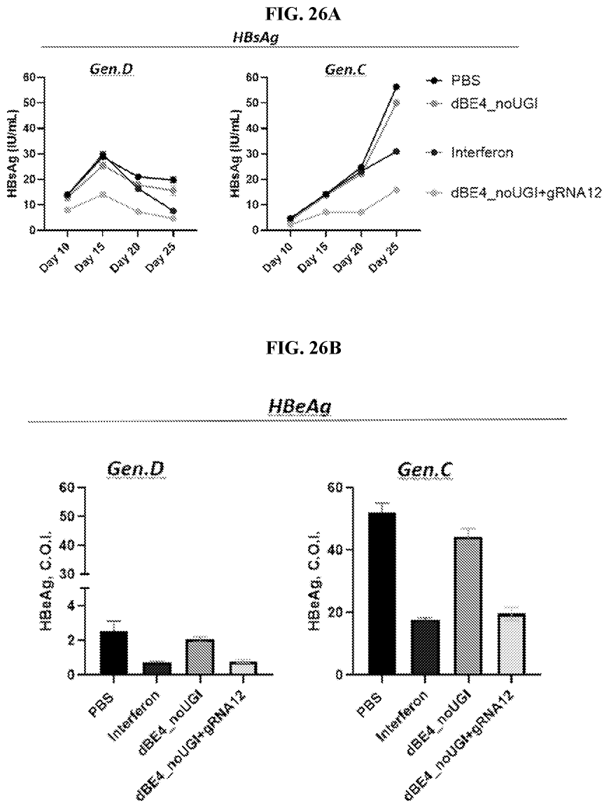

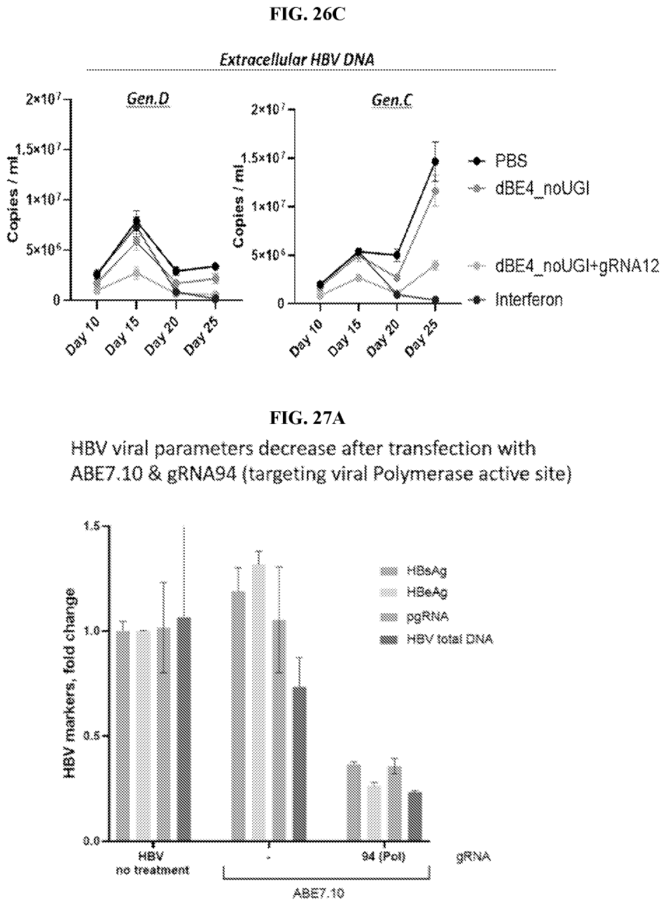

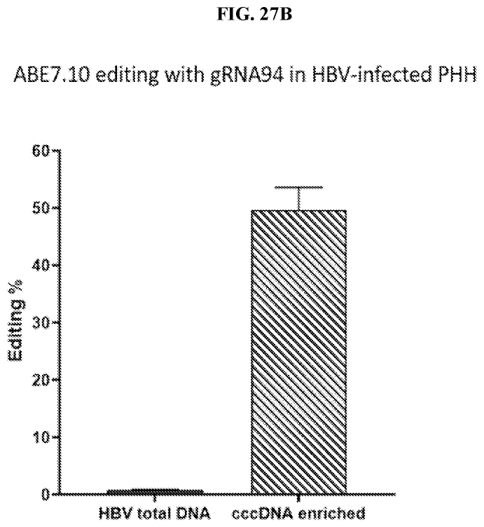

The base editor BE4_noUGI possesses a nickase activity that may lead to a double stranded break (dsbreaks) in the targeted DNA site (Komor A C et al., 2016). Double stranded breaks in cccDNA can further lead to integration of the HBV DNA into the human genome, which should be avoided. In order to minimize the possibility of generating dsbreaks during the base editing process, mutated BE4_noUGI (H840A), i.e., dBE4_noUGI, which does not have a nickase activity and only possesses deaminase activity, was generated and assessed. The base editing activity of dBE4_noUGI was evaluated in conjunction with gRNA12 (Pol/S) using HBV-infected PHH in a long-term (25-day) experiment. While transfection of base editor alone (no gRNA) did not lead to a significant reduction in the assessed viral parameters, transfection of the dBE4_noUGI and gRNA12 led to a robust HBV inhibition, as determined by measuring the levels of the viral parameters HBsAg, HBV DNA, and HBeAg. (FIGS. 25A-25C).

In addition, the antiviral activity of interferon, which is known for use in treating HBV, was compared with the base editing activity of the base editor dBE4_noUGI and gRNA in inhibiting HBV in a long-term experiment using HBV-infected PHH. While interferon can effectively treat HBV in some patients, this agent is also known to be toxic and can cause a number of adverse side-effects. The result of the experiment demonstrated that both interferon and base editing using dBE4_noUGI+gRNA12 decreased the HBV viral parameters to comparable levels. (FIGS. 25A-25C). Cell viability and metabolic activity were also evaluated by testing levels of albumin secreted into the cell medium at the end of the experiment. Interferon treatment led to a significant reduction of secreted albumin, which is indicative of the toxicity of interferon treatment. In contrast to interferon treatment, base editing by the dBE4_noUGI and gRNA (e.g., gRNA12) caused minimal reduction of secreted albumin levels, thus indicating a lack of toxicity associated with the use of the base editing approach. (FIG. 25D).

### Example 13 Base Editing Reduces Viral Parameters for HBV of Genotypes D and C in HBV-Infected PHH (Long-Term Experiment)

Experiments were conducted to determine whether the base editing approach resulted in similar anti-viral efficacy for HBV of different genotypes. Accordingly, dBE4_noUGI and gRNA12 (targeting the intersection of HBV polymerase and S gene (P/S)) were assessed in HBV-infected PHH co-cultures in parallel experiments in which the PHH were infected with either with HBV of genotype D (Gen.D) or HBV of genotype C (Gen.C). Infection of PHH with HBV of genotype C (no treatment condition) resulted in a higher viral load at the termination of the experiment. Despite the higher levels of the viral parameters assessed in the experiments, transfection with the base editor dBE4_no UGI and gRNA12 showed robust HBV inhibition of both HBV Gen.D and HBV Gen.C compared to the controls. (FIGS. 26A-26C). The results support the use of base editing and the base editor systems described herein for the treatment of infection caused by HBV of different genotypes.

### Example 14 Transfection with ABE7.10 and HBV-Specific gRNA Targeting the HBV Polymerase Active Site Reduces HBV Markers in HBV-Infected PHH

Adenine base editors (ABEs) possess robust base editing activity, while showing minimal off-target effects. (Gaudelli, N. M., et al., “Programmable base editing of A·T to G·C in genomic DNA without DNA cleavage” Nature 551, 464-471 (2017). While endogenous cytidine deaminases have been reported to deaminate cccDNA, no such mechanism or activity has been shown for adenine deaminases. To determine whether HBV editing with ABE resulted in inhibiting virus replication in HBV-infected primary hepatocytes, the adenine deaminase-containing base editor ABE7.10, as described herein, was assessed in conjunction with a gRNA in experiments involving the use of the HBV-infected PHH co-culture system. Accordingly, HBV-infected PHH cultures were transfected with ABE7.10 mRNA and gRNA94, which was designed to introduce a silent mutation in the HBV polymerase active site, as described in Example 10 supra and FIG. 19. An analysis of the experimental results showed that robust reductions of the viral parameters HBsAg, HBeAg, pgRNA, and HBV total DNA were detected with the use of the base editor ABE7.10 mRNA and gRNA94 (targeting the HBV polymerase active site), in combination, compared with ABE7.10 mRNA used alone, which did not cause a decrease in the viral parameters (FIG. 27A). Next Generation Sequencing (NGS) was performed on the total DNA purified from HBV-infected PHH and on the same samples enriched for cccDNA through ExoI/ExoIII digestion. Approximately 50% base editing by ABE7.10 mRNA and gRNA94 was detected in cccDNA-enriched samples, which indicated successful cccDNA editing. An absence of base editing in total DNA purified from HBV-PHH is indicative of the inability of the edited cccDNA to propagate into a replication competent viral particle. (FIG. 27B). These data in combination demonstrate that ABEs serve as efficient antiviral reagents for reducing HBV viral load.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method of editing a nucleobase of a hepatitis B virus (HBV) genome, the method comprising contacting the HBV genome with one or more guide RNAs and a base editor comprising a polynucleotide programmable DNA binding domain and an adenosine deaminase or cytidine deaminase domain, wherein said guide RNA targets said base editor to effect an alteration of the nucleobase of the HBV genome.

2. The method of claim 1, wherein the nucleobase is in a polynucleotide encoding an HBV protein.

3. The method of claim 1, wherein the contacting is in a eukaryotic cell, a mammalian cell, or a human cell.

4. The method of claim 1, wherein the cell is in vivo or ex vivo.

5. The method of claim 1, wherein the cytidine deaminase converts a target C to U in the HBV genome.

6. The method of claim 1, wherein the cytidine deaminase converts a target C·G to T·A in the polynucleotide encoding the HBV protein.

7. The method of claim 1, wherein the adenosine deaminase converts a target A·T to G·C in the polynucleotide encoding the HBV protein.

8. The method of claim 2, wherein alteration of the nucleobase in the polynucleotide encoding the HBV protein results in a premature termination codon.

9. The method of claim 8, wherein the alteration of the nucleobase results in an R87* or W120* termination in an HBV X protein.

10. The method of claim 8, wherein the alteration of the nucleobase results in an W35* or W36* in an HBV S protein.

11. The method of claim 2, wherein the alteration of the HBV polynucleotide is a missense mutation.

12. The method of claim 11, wherein the missense mutation is in an HBV pol gene.

13. The method of claim 12, wherein the missense mutation results in an E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P in an HBV polymerase protein encoded by the HBV pol gene.

14. The method of claim 11, wherein the missense mutation is in an HBV core gene.

15. The method of claim 14, wherein the missense mutation results in a T160A, T160A, P161F, S162L, C183R, or *184Q in an HBV core protein encoded by the HBV core gene.

16. The method of claim 11, wherein the missense mutation is in an HBV X gene.

17. The method of claim 16, wherein the missense mutation results in a H86R, W120R, E122K, E121K, or L141P in an HBV X protein encoded by the HBV X gene.

18. The method of claim 11, wherein the missense mutation is in an HBV S gene.

19. The method of claim 18, wherein the missense mutation results in a S38F, L39F, W35R, W36R, T37I, T37A, R78Q, S34L, F80P, or D33G in an HBV S protein encoded by the HBV S gene.

20-23. (canceled)

24. The method of claim 1, wherein the polynucleotide programmable DNA binding domain is a nuclease inactive or nickase variant.

25-38. (canceled)

39. A method of treating hepatitis B virus (HBV) infection in a subject, comprising administering to a subject in need thereof one or more polynucleotides encoding a polynucleotide programmable DNA binding domain and a base editor domain that is an adenosine deaminase or a cytidine deaminase domain, and one or more guide polynucleotides that target the base editor domain to effect an A·T to G·C, C·G to T·A, or C·G to U·A alteration of the nucleic acid sequence encoding an HBV polypeptide.

40-77. (canceled)

78. A composition comprising a base editor bound to a guide RNA, wherein the guide RNA comprises a nucleic acid sequence that is complementary to an HBV gene.

79-96. (canceled)

97. A pharmaceutical composition for the treatment of HBV infection comprising
(i) a base editor, or a nucleic acid encoding the base editor, and one or more guide RNAs (gRNA) comprising a nucleic acid sequence complementary to an HBV gene in a pharmaceutically acceptable excipient.

98-105. (canceled)

106. A method of treating HBV infection, the method comprising administering to a subject in need thereof the pharmaceutical composition of claim 97.

107. (canceled)

108. An HBV genome comprising an alteration selected from the group consisting of:
a premature termination codon introducing a R87STOP or W120STOP in the X gene;
a premature termination codon introducing a W35STOP or W36STOP in the S gene;
a missense mutation in the HBV pol gene that introduces a E24G, L25F, P26F, R27C, V48A, V48I, S382F, V378I, V378A, V379I, V379A, L377F, D380G, D380N, F381P, R376G, A422T, F423P, A432V, M433V, P434S, D540G, A688V, D689G, A717T, E718K, P713S, P713L, or L719P in HBV polymerase;
a missense mutation is in the HBV core gene that introduces a T160A, T160A, P161F, S162L, C183R, or STOP184Q in the HBV Core polypeptide;
a missense mutation is in the X gene that introduces a H86R, W120R, E122K, E121K, or L141P in the HBV X polypeptide; and
a missense mutation in the S gene that introduces a S38F, L39F, W35R, W36R, T37I, T37A, R78Q, S34L, F80P, or D33G in the HBV S polypeptide.

109-115. (canceled)

116. A guide RNA comprising a nucleic acid sequence that is complementary to an HBV gene.

117-122. (canceled)

123. The guide RNA of claim 116 comprising a nucleic acid selected from the group consisting of, from 5′ to 3′, UCAAUCCCAACAAGGACACC; GGGAACAAGAUCUACAGCAU; AAGCCCAGGAUGAUGGGAUG; CUGCCAACUGGAUCCUGCGC; GACACAUCCAGCGAUAACCA; GCUGCCAACUGGAUCCUGCG; UAUGGAUGAUGUGGUAUUGG; CCAUGCCCCAAAGCCACCCA; AAGCCACCCAAGGCACAGCU; GAGAAGUCCACCACGAGUCU; CUUCUCUCAAUUUUCUAGGG; GACGACGAGGCAGGUCCCCU; CCCAACAAGGACACCUGGCC; UGCCAACUGGAUCCUGCGCG; AGGAGUUCCGCAGUAUGGAU; CCGCAGUAUGGAUCGGCAGA; CCUCUGCCGAUCCAUACUGC; CGCCCACCGAAUGUUGCCCA; GACUUCUCUCAAUUUUCUAG; GUUCCGCAGUAUGGAUCGGC; UACUAACAUUGAGGUUCCCG; UCCGCAGUAUGGAUCGGCAG; UCCUCUGCCGAUCCAUACUG; GUAGCUCCAAAUUCUUUAUA; and AAUCCACACUCCGAAAGACA.

124. A pharmaceutical composition comprising (i) a nucleic acid encoding a base editor; and (ii) the guide RNA of claim 116.

125. (canceled)
